(19) United States
(12) Patent Application Publication
(10) Pub. No.: US 2012/0238549 A1
(43) Pub. Date: Sep. 20, 2012

(54) Nuclear Hormone Receptor Modulators

(75) Inventors: Kevin P. Cusack, Holden, MA (US); Thomas D. Gordon, Medway, MA (US); David C. Ihle, Worcester, MA (US); Martin E. Hayes, Lowell, MA (US); Eric C. Breinlinger, Charlton, MA (US); Anna M. Ericsson, Shrewsbury, MA (US); Bin Li, Ashland, MA (US); Lei Wang, Acton, MA (US); Gloria Y. Martinez, Shrewsbury, MA (US); Andrew Burchat, Shrewsbury, MA (US); Adrian D. Hobson, Shrewsbury, MA (US); Kelly D. Mullen, Charlton, MA (US); Michael Friedman, Brookline, MA (US); Michael J. Morytko, Framingham, MA (US)

(73) Assignee: ABBOTT LABORATORIES, Abbott Park, IL (US)

(21) Appl. No.: 13/420,957

(22) Filed: Mar. 15, 2012

Related U.S. Application Data
(60) Provisional application No. 61/452,790, filed on Mar. 15, 2011.
(60) Provisional application No. 61/565,030, filed on Nov. 30, 2011.

(51) Int. Cl. A61K 31/122; C07D 213/74; A61K 31/44; C07D 401/12; A61K 31/55; C07C 45/61; C07C 45/62; C07D 453/04; A61P 5/00; A61P 25/30; A61P 37/08; A61P 25/28; A61P 7/10; A61P 25/22; A61P 11/06; A61P 37/00; A61P 31/14; A61P 9/00; A61P 13/12; A61P 27/02; A61P 1/16; A61P 1/00; A61P 25/24; A61P 3/10; A61P 27/06; A61P 29/00; A61P 31/18; A61P 9/12; A61P 35/00; A61P 37/02; A61P 5/48; A61P 35/02; A61P 19/04; A61P 25/00; A61P 3/04; A61P 37/06; A61P 19/10; A61P 19/02; A61P 25/16; A61P 17/06; A61P 25/18; A61P 3/00; A61P 1/04; A61P 17/02; C07C 49/755

(52) U.S. Cl. 514/212.04; 568/326; 514/680; 546/285; 514/352; 540/522; 568/313; 568/309; 568/312; 546/134; 568/315; 568/311; 568/316

(57) ABSTRACT

The invention provides a compound of Formula (I)

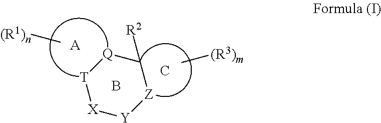

pharmaceutically acceptable salts, pro-drugs, biologically active metabolites, stereoisomers and isomers thereof wherein the variable are defined herein. The compounds of the invention are useful for treating immunological and oncological conditions.

### CROSS REFERENCE TO RELATED APPLICATION

This application claims priority to U.S. Provisional Application Ser. No. 61/452,790 filed on Mar. 15, 2011 and to U.S. Provisional Application Ser. No. 61/565,030 filed on Nov. 30, 2011, the contents of which are incorporated herein.

### BACKGROUND OF THE INVENTION

The invention provides a novel class of compounds, pharmaceutical compositions comprising such compounds and methods of using such compounds to treat or prevent diseases or disorders associated with modulation of the glucocorticoid receptor. Modulators of the glucocorticoid receptor are useful in the treatment of certain inflammatory related conditions.

Intracellular receptors (IR's) are a class of structurally related proteins involved in the regulation of gene expression. The steroid hormone receptors are a subset of this superfamily whose natural ligands are typically comprised of endogenous steroids such as estradiol, progesterone, and cortisol. Man-made ligands to these receptors play an important role in human health and of these receptors the glucocorticoid receptor (GR) has an essential role in regulating human physiology and immune response.

Steroids which interact with GR have been shown to be potent anti-inflammatory agents. Examples include the glucocorticoid (GC) agonists dexamethasone, prednisone, and prednisolone. The utility of GC agonists in a chronic setting has been limited however due to multiple serious side effects such as osteoporosis, effects on glucose metabolism (diabetogenic), skin thinning, fluid homeostasis and depression for example. [Expert Opinion on Therapeutic Patents (2000) 10(1), 117] These effects are believed to be the result of cross-reactivity with other steroid receptors such as estrogen, progesterone, androgen, and mineralocorticoid receptors which have somewhat homologous ligand binding domains, and/or the inability to selectively modulate downstream signaling. Identification of a selective glucocorticoid receptor modulator (SGRM) that is efficacious with reduced side-effects could fulfill an unmet medical need.

Selective GR modulators (e.g. repressors, agonists, partial agonists and antagonists) of the present disclosure can be used to influence the basic, life-sustaining systems of the body, including carbohydrate, protein and lipid metabolism, and the functions of the cardiovascular, kidney, central nervous, immune, skeletal muscle, and other organ and tissue systems. In this regard, GR modulators have proven useful in the treatment of inflammation, tissue rejection, auto-immunity, various malignancies, such as leukemias and lymphomas, Cushing's syndrome, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, modulation of the Th1/Th2 cytokine balance, chronic kidney disease, stroke and spinal cord injury, hypercalcemia, hypergylcemia, acute adrenal insufficiency, chronic primary adrenal insufficiency, secondary adrenal insufficiency, congenital adrenal hyperplasia, cerebral edema, thrombocytopenia, and Little's syndrome. GR modulators are especially useful in disease states involving systemic inflammation such as inflammatory bowel disease, systemic lupus erythematosus, polyartitis nodosa, Wegener's granulomatosis, giant cell arteritis, rheumatoid arthritis, osteoarthritis, hay fever, allergic rhinitis, urticaria, angioneurotic edema, chronic obstructive pulmonary disease, asthma, tendonitis, bursitis, Crohn's disease, ulcerative colitis, autoimmune chronic active hepatitis, organ transplantation, hepatitis, cirrhosis, juvenile rheumatoid arthritis, juvenile idiopathic arthritis, ankylosing spondylitis, psoriasis, plaque psoriasis, and psoriatic arthritis. GR active compounds have also been used as immunostimulants and repressors, and as wound healing and tissue repair agents.

GR modulators have also found use in a variety of topical diseases such as inflammatory scalp alopecia, panniculitis, psoriasis, discoid lupus erythematosus, inflamed cysts, atopic dermatitis, pyoderma gangrenosum, pemphigus vulgaris, bullous pemphigoid, systemic lupus erythematosus, dermatomyositis, herpes gestationis, eosinophilic fasciitis, relapsing polychondritis, inflammatory vasculitis, sarcoidosis, Sweet's disease, type 1 reactive leprosy, capillary hemangiomas, contact dermatitis, atopic dermatitis, lichen planus, exfoliative dermatitus, erythema nodosum, acne, hirsutism, toxic epidermal necrolysis, erythema multiform, cutaneous T-cell lymphoma and ocular diseases. Selective antagonists of the glucocorticoid receptor have been unsuccessfully pursued for decades. These agents would potentially find application in several disease states associated with Human Immunodeficiency Virus (HIV), cell apoptosis, and cancer including, but not limited to, Kaposi's sarcoma, immune system activation and modulation, desensitization of inflammatory responses, IL-1 expression, anti-retroviral therapy, natural killer cell development, lymphocytic leukemia, and treatment of retinitis pigmentosa. Cogitive and behavioral processes are also susceptible to glucocorticoid therapy where antagonists would potentially be useful in the treatment of processes such as cognitive performance, memory and learning enhancement, depression, addiction, mood disorders, chronic fatigue syndrome, schizophrenia, stroke, sleep disorders, and anxiety.

### SUMMARY OF THE INVENTION

In a first embodiment the invention provides a compound of Formula (I)

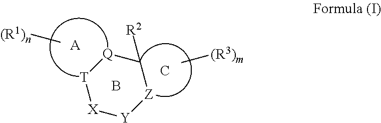

pharmaceutically acceptable salts, pro-drugs, biologically active metabolites, isomers, and stereoisomers wherein
Ring A is optionally substituted aryl, optionally substituted saturated or partially unsaturated (C₅-C₆)carbocyclyl or optionally substituted heteroaryl;
Ring C is optionally substituted saturated or partially unsaturated (C₅-C₆)carbocyclyl or optionally substituted heterocyclyl;
Q and T are independently C or N, provided that both are not N;
Ring B is a seven membered ring wherein
   X is —C(R⁵)₂—, —C(R⁵)—, —C(═O)—, —N(R^{a})—, —O—, —S—, —S(O)—, or —S(O)₂—; or
   when X is —C(R⁵)₂—, it can form a cyclopropyl ring spiro to the carbon atom to which it is attached;
   Y is —C(R⁵)₂C(R⁵)₂—, —C(R⁵)C(R⁵)₂—, —C(R⁵)₂C(R⁵)—, —OC(R⁵)₂—, —N(R^{a})C(R⁵)₂—, —C(R⁵)₂N(R^{a})—, —C(═O)C(R⁵)₂—, —C(R⁵)₂C(═O)—, —O—C(═O)—, —C(═O)—O—, or —C(R⁵)₂—O—; or
   Y is —C(R⁵)₂— when Q or T is N;
   Z is CR⁴ or N; or
Ring B is a six membered ring wherein
   Y is —C(R⁵)₂;
   Q or T must be N;
   Z is CR⁴ or N; or
   when X is —C(R⁵)₂—, it can form a cyclopropyl ring spiro to the carbon atom to which it is attached;
provided that X—Y or Y—Z do not form O—O, N—N, N—O, C(═O)—C(═O), N—C—O or O—C—O bonds; and
provided that in X—Y a sulfur atom is not adjacent to an oxygen atom or —C(═O);
provided that X—Y does not form —O—C(R⁵)₂—O—, —N—C(R⁵)₂—O— or —S—C(R⁵)₂—O—;

R′ is H, Br, Cl, F, —COOR^{a}, —OR^{a}, —O-optionally substituted (C₁-C₃)alkylene-optionally substituted aryl, —O-optionally substituted (C₁-C₃)alkylene-optionally substituted heteroaryl, —O-optionally substituted (C₁-C₃)alkylene-optionally substituted heterocyclyl, optionally substituted (C₁-C₃)alkyl, optionally substituted aryl, optionally substituted (C₃-C₆)cycloalkyl, optionally substituted heteroaryl, optionally substituted heterocyclyl, —C(O)N(R^{a})(CH₂)ᵣ—R^{b}, —N(R^{a})C(O)(CH₂)ᵣ—R^{b}, —S(O)₂N(R^{a})—R^{b}, —N(R^{a})S(O)₂—R^{b}, —O—S(O)₂—CF₃, —N(R^{a})-optionally substituted (C₃-C₆)cycloalkyl, —N(R^{a})-optionally substituted heterocyclyl, —N(R^{a})-optionally substituted heteroaryl, —N(R^{a})-optionally substituted aryl,

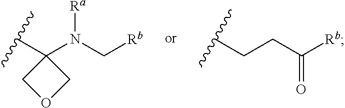

R² is —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl;

R³ is independently H, deuterium, —CD₃, —CF₃, optionally substituted (C₂-C₆)alkynyl, oxo, —OR^{a}, —OP(═O)(OH)(OH), optionally substituted (C₁-C₄)alkyl, —(C(R^{a})₂)ᵣ-optionally substituted (C₃-C₆)cycloalkyl, —(C(R^{a})₂)ᵣ-optionally substituted aryl, —(C(R^{a})₂)ᵣ-optionally substituted heteroaryl, —(C(R^{a})₂)ᵣ—N(R^{a})-optionally substituted heteroaryl, a carbocyclic or heterocyclic spirocyclic moiety attached to ring C;

R⁴ is H, optionally substituted (C₁-C₃)alkyl, OH or —O-optionally substituted (C₁-C₃)alkyl;

R⁵ is independently H, F, N(R^{a}), OR^{a}, optionally substituted (C₃-C₆)cycloalkyl, or optionally substituted (C₁-C₃)alkyl;

R^{a} is independently H, optionally substituted (C₃-C₆)cycloalkyl or optionally substituted (C₁-C₃)alkyl;

R^{b} is H, optionally substituted (C₁-C₃)alkyl, optionally substituted aryl, optionally substituted (C₃-C₆)cycloalkyl, optionally substituted heteroaryl or optionally substituted heterocyclyl;

m is 1, 2, 3 or 4;

n is 1, 2, 3 or 4; and

r is independently 0, 1 or 2.

In a second embodiment the invention provides a compound according first embodiment wherein the compound is of Formula (I)a or Formula (I)b

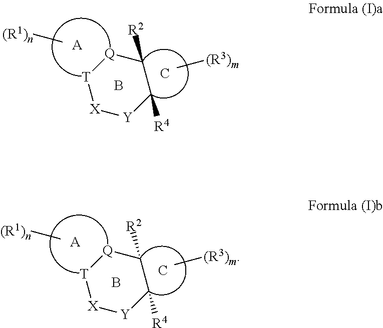

In a third embodiment the invention provides a compound according to any of the foregoing embodiments wherein Ring A is optionally substituted phenyl, optionally substituted pyrrolyl, or optionally substituted pyrazolyl.

In a fourth embodiment the invention provides a compound according to any of the foregoing embodiments wherein Ring C is optionally substituted cyclohexyl or optionally substituted cyclohexenyl.

In a fifth embodiment the invention provides a compound according to any of the foregoing embodiments wherein X is —C(R⁵)₂—, —C(R⁵)—, —C(═O)—, —O— or —N(R^{a})—.

In a sixth embodiment the invention provides a compound according to any of the foregoing embodiments wherein R¹ is —COOR^{a}, OR^{a}, optionally substituted (C₁-C₃)alkyl, —C(O)N(R^{a})(CH₂)ᵣ—R^{b}, —N(R^{a})C(O)(CH₂)ᵣ—R^{b}, optionally substituted azabenzimidazolyl, optionally substituted benzimidazolyl, —O-optionally substituted (C₁-C₃)alkylene-optionally substituted phenyl, or —O-optionally substituted (C₁-C₃)alkylene-optionally substituted pyridinyl.

In a seventh embodiment the invention provides a compound according to any of the foregoing embodiments wherein R² is —CH₂CF₃, —(CH₂)ᵣ-optionally substituted aryl, or optionally substituted (C₁-C₃)alkyl.

In an eighth embodiment the invention provides a compound according to any of the foregoing embodiments wherein R³ is independently H, —CF₃, —C≡CH₃, oxo, —OR^{a}, —OP(═O)(OH)(OH), optionally substituted (C₁-C₄)alkyl, —(C(R^{a})₂)ᵣ-optionally substituted (C₃-C₆)cycloalkyl, or —(CH₂)ᵣ-optionally substituted aryl.

In a ninth embodiment the invention provides a compound according to any of the foregoing embodiments wherein R³ is independently H, —CF₃, —C≡CH₃, oxo, —OR^{a}, optionally substituted (C₁-C₄)alkyl, —CH₂-optionally substituted cyclopropyl, —CH₂-optionally substituted phenyl, or -optionally substituted phenyl.

In a tenth embodiment the invention provides a compound according to any of the foregoing embodiments wherein R^{b} is H, optionally substituted azetidinyl, optionally substituted phenyl, optionally substituted piperidinyl, optionally substituted pyrimidinyl, optionally substituted pyridinyl, optionally substituted pyrazolyl, optionally substituted pyrrolidinyl or optionally substituted tetrazolyl.

In an eleventh embodiment the invention provides a compound according to any of the foregoing embodiments wherein Q is C.

In a twelfth embodiment the invention provides a compound according to any of the foregoing embodiments wherein T is C.

In a thirteenth embodiment the invention provides a compound according to any of the foregoing embodiments wherein the compound is
(4aR,11bS)-11b-benzyl-9-hydroxy-1,2,4,4a,5,6,7,11b-octahydro-dibenzo[a,c]cyclohepten-3-one; compound with (4aS,11bR)-11b-benzyl-9-hydroxy-1,2,4,4a,5,6,7,11b-octahydro-dibenzo[a,c]cyclohepten-3-one;
(3R,4aS,11bS)-11b-Benzyl-3-methyl-2,3,4,4a,5,6,7,11b-octahydro-1H-dibenzo[a,c]cycloheptene-3,9-diol; compound with (3S,4aR,11bR)-11b-benzyl-3-methyl-2,3,4,4a,5,6,7,11b-octahydro-1H-dibenzo[a,c]cycloheptene-3,9-diol;
(3R,4aR,11bR)-11b-benzyl-3-methyl-2,3,4,4a,5,6,7,11b-octahydro-1H-dibenzo[a,c]cycloheptene-3,9-diol; compound with (3S,4aS,11bS)-11b-benzyl-3-methyl-2,3,4,4a,5,6,7,11b-octahydro-1H-dibenzo[a,c]cycloheptene-3,9-diol;
(7aS,11aS)-11a-Benzyl-9-oxo-6,7,7a,8,9,10,11,11a-octahydro-5H-dibenzo[a,c]cycloheptene-3-carboxylic acid (2-methyl-pyridin-3-yl)-amide; compound with (7aR,11aR)-11a-benzyl-9-oxo-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9S,11aS)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide;
(7aR,9S,11aR)-11a-benzyl-9-ethyl-9-hydroxy-6,7,7a,8,9,10,11,11a-octahydro-5H-dibenzo[a,c]]cycloheptene-3-carboxylic acid (2-methyl-pyridin-3-yl)-amide;
(7aS,9S,11aS)-11a-Benzyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-11a-benzyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-6,7,7a,8,9,10,11,11a-octahydro-5H-dibenzo[a,c]]cycloheptene-3-carboxylic acid (2-methyl-pyridin-3-yl)-amide; compound with (7aR,9S,11aR)-11a-benzyl-9-hydroxy-6,7,7a,8,9,10,11,11a-octahydro-5H-dibenzo[a,c]]cycloheptene-3-carboxylic acid (2-methyl-pyridin-3-yl)-amide;
(7aS,9R,11aS)-11a-Benzyl-9-hydroxy-9-methoxymethyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-hydroxy-9-methoxymethyl-6,7,7a,8,9,10,11,11a-octahydro-5H-dibenzo[a,c]cycloheptene-3-carboxylic acid (2-methyl-pyridin-3-yl)-amide;
(7aR,11aS)-11a-Benzyl-9-hydroxy-N-(2-methylpyridin-3-yl)-6-oxo-9-(trifluoromethyl)-6,7,7a,8,9,10,11,11a-octahydro-5H-dibenzo[b,d]azepine-3-carboxamide;
(4aS,11bS)-11b-benzyl-3-hydroxy-N-(2-methylpyridin-3-yl)-7-oxo-3-(trifluoromethyl)-2,3,4,4 a,5,6,7,11b-octahydro-1H-dibenzo[c,e]azepine-9-carboxamide;
(7aS,9R,11aS)-11a-Benzyl-9-ethyl-9-hydroxy-5-oxo-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-ethyl-9-hydroxy-5-oxo-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-ethyl-9-hydroxy-5-oxo-6,7,7a,8,9,10,11,11a-octahydro-5H-dibenzo[a,c]cycloheptene-3-carboxylic acid (2-methyl-pyridin-3-yl)-amide;
(7aR,9S,11aR)-11a-benzyl-9-ethyl-9-hydroxy-5-oxo-6,7,7a,8,9,10,11,11a-octahydro-5H-dibenzo[a,c]cycloheptene-3-carboxylic acid (2-methyl-pyridin-3-yl)-amide;
(7aR,9S,11aS)-9-Ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide; compound with (7aS,9R,11aR)-9-ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide;
(7aR,9R,11aS)-9-ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide; compound with (7aS,9S,11aR)-9-ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide;
(3S,4aS,11bS)-11b-Benzyl-3-prop-1-ynyl-2,3,4,4a,5,6,7,11b-octahydro-1H-dibenzo[a,c]cycloheptene-3,9-diol; compound with (3R,4aR,11bR)-11b-benzyl-3-prop-1-ynyl-2,3,4,4a,5,6,7,11b-octahydro-1H-dibenzo[a,c]cycloheptene-3,9-diol;
(7aS,9S,11aS)-11a-Benzyl-9-hydroxy-9-prop-1-ynyl-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-11a-benzyl-9-hydroxy-9-prop-1-ynyl-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-propyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-hydroxy-9-propyl-6,7,7a,8,9,10,11,11a-octahydro-5H-dibenzo[a,c]cycloheptene-3-carboxylic acid (2-methyl-pyridin-3-yl)-amide;
(7aS,9S,11aS)-11a-Benzyl-9-ethynyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aS,9S,11aS)-11a-benzyl-9-ethynyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-ethoxymethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-ethoxymethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aR)-9-Benzyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide; compound with (7aR,9S,11aS)-9-benzyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide;
(7aS,9S,11aR)-9-benzyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide; compound with (7aR,9R,11aS)-9-benzyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide;
(6aS,8R,10aS)-10a-Benzyl-8-ethyl-1-methyl-1,4,5,6,6a,7,8,9,10,10a-decahydro-1,2-diaza-benzo[e]azulen-8-ol; compound with (6aR,8S,10aR)-10a-benzyl-8-ethyl-1-methyl-1,4,5,6,6a,7,8,9,10,10a-decahydro-1,2-diaza-benzo[e]azulen-8-ol;
(6aS,8S,10aS)-10a-Benzyl-8-ethyl-1-methyl-1,4,5,6,6a,7,8,9,10,10a-decahydro-1,2-diaza-benzo[e]azulen-8-ol; compound with (6aR,8R,10aR)-10a-benzyl-8-ethyl-1-methyl-1,4,5,6,6a,7,8,9,10,10a-decahydro-1,2-diaza-benzo[e]azulen-8-ol;
(6aS,8R,10aS)-10a-Benzyl-8-ethyl-2-methyl-2,4,5,6,6a,7,8,9,10,10a-decahydro-1,2-diaza-benzo[e]azulen-8-ol; compound with (6aR,8S,10aR)-10a-benzyl-8-ethyl-2-methyl-2,4,5,6,6a,7,8,9,10,10a-decahydro-1,2-diaza-benzo[e]azulen-8-ol;
(6aS,8S,10aS)-10a-benzyl-8-ethyl-2-methyl-2,4,5,6,6a,7,8,9,10,10a-decahydro-1,2-diaza-benzo[e]azulen-8-ol; compound with (6aR,8R,10aR)-10a-benzyl-8-ethyl-2-methyl-2,4,5,6,6a,7,8,9,10,10a-decahydro-1,2-diaza-benzo[e]azulen-8-ol;
(2R,3R,4aS,11bR)-11b-Benzyl-3-phenyl-2,3,4,4a,5,6,7,11b-octahydro-1H-dibenzo[a,c]cycloheptene-2,3,9-triol compound with (2S,3S,4aR,11bS)-11b-benzyl-3-phenyl-2,3,4,4a,5,6,7,11b-octahydro-1H-dibenzo[a,c]cycloheptene-2,3,9-triol;
(7aS,9R,10R,11aR)-11a-Benzyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide; compound with (7aR,9S,10S,11aS)-11a-benzyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide;
(7aR,9S,11aS)-11a-Ethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide;
(7aS,9R,11aR)-11a-Ethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide;
(7aR,9S,11aS)-9,11a-Diethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aR)-9,11a-Diethyl-9-hydroxy-6,7,7a,8,9,10,11,11a-octahydro-5H-dibenzo[a,c]cycloheptene-3-carboxylic acid (2-methyl-pyridin-3-yl)-amide;
7aR,9R,11aS)-11a-Ethyl-9-hydroxy-9-isobutyl-6,7,7a,8,9,10,11,11a-octahydro-5H-dibenzo[a,c]cycloheptene-3-carboxylic acid (2-methyl-pyridin-3-yl)-amide;
(7aS,9S,11aR)-11a-Ethyl-9-hydroxy-9-isobutyl-6,7,7a,8,9,10,11,11a-octahydro-5H-dibenzo[a,c]cycloheptene-3-carboxylic acid (2-methyl-pyridin-3-yl)-amide;
(7aR,9R,11aS)-9-Cyclopropylmethyl-11a-ethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9S,11aR)-9-Cyclopropylmethyl-11a-ethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aS)-9-Hydroxy-9-propyl-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-9-hydroxy-9-propyl-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide;
(7aS,9S,11aR)-11a-Ethyl-9-hydroxy-9-isobutyl-6,7,7a,8,9,10,11,11a-octahydro-5-oxa-dibenzo[a,c]cycloheptene-3-carboxylic acid (2-methyl-pyridin-3-yl)-amide; compound with (7aR,9R,11aS)-11a-Ethyl-9-hydroxy-9-isobutyl-6,7,7a,8,9,10,11,11a-octahydro-5-oxa-dibenzo[a,c]cycloheptene-3-carboxylic acid (2-methyl-pyridin-3-yl)-amide;
(7aS,9S,11aR)-9-Cyclopropylmethyl-11a-ethyl-9-hydroxy-6,7,7a,8,9,10,11,11a-octahydro-5-oxa-dibenzo[a,c]cycloheptene-3-carboxylic acid (2-methyl-pyridin-3-yl)-amide; compound with (7aR,9R,11aS)-9-Cyclopropylmethyl-11a-ethyl-9-hydroxy-6,7,7a,8,9,10,11,11a-octahydro-5-oxa-dibenzo[a,c]cycloheptene-3-carboxylic acid (2-methyl-pyridin-3-yl)-amide;
(7aS,9S,11aR)-11a-Ethyl-9-hydroxy-9-isobutyl-6,7,7a,8,9,10,11,11a-octahydro-5-oxa-dibenzo[a,c]cycloheptene-3-carboxylic acid (2-methyl-pyridin-3-yl)-amide;
(7aR,9R,11aS)-11a-Ethyl-9-hydroxy-9-isobutyl-6,7,7a,8,9,10,11,11a-octahydro-5-oxa-dibenzo[a,c]cycloheptene-3-carboxylic acid (2-methyl-pyridin-3-yl)-amide;
(7aS,9S,11aR)-9-Cyclopropylmethyl-11a-ethyl-9-hydroxy-6,7,7a,8,9,10,11,11a-octahydro-5-oxa-dibenzo[a,c]cycloheptene-3-carboxylic acid (2-methyl-pyridin-3-yl)-amide;
(7aR,9R,11aS)-9-Cyclopropylmethyl-11a-ethyl-9-hydroxy-6,7,7a,8,9,10,11,11a-octahydro-5-oxa-dibenzo[a,c]cycloheptene-3-carboxylic acid (2-methyl-pyridin-3-yl)-amide;
(7aS,9R,11aS)-9-Hydroxy-9-propyl-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide;
(7aR,9S,11aR)-9-Hydroxy-9-propyl-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide;
(7aR,9S,11aS)-11a-Ethyl-9-hydroxy-9-methyl-6,7,7a,8,9,10,11,11a-octahydro-5H-dibenzo[a,c]cycloheptene-3-carboxylic acid (2-methyl-pyridin-3-yl)-amide;
(7aS,9R,11aR)-11a-ethyl-9-hydroxy-9-methyl-6,7,7a,8,9,10,11,11a-octahydro-5H-dibenzo[a,c]cycloheptene-3-carboxylic acid (2-methyl-pyridin-3-yl)-amide;
(7aR,9R,11aS)-11a-Ethyl-9-hydroxy-5-oxo-9-propyl-6,7,7a,8,9,10,11,11a-octahydro-5H-dibenzo[a,c]cycloheptene-3-carboxylic acid (2-methyl-pyridin-3-yl)-amide;
(7aS,9S,11aR)-11a-Ethyl-9-hydroxy-5-oxo-9-propyl-6,7,7a,8,9,10,11,11a-octahydro-5H-dibenzo[a,c]cycloheptene-3-carboxylic acid (2-methyl-pyridin-3-yl)-amide;
(7aS,9S,11aS)-9-Hydroxy-9-isobutyl-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-9-hydroxy-9-isobutyl-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide;
(7aS,9R,11aR)-9-Cyanomethyl-11a-ethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aS)-9-cyanomethyl-11a-ethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-cyanomethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-Benzyl-9-cyanomethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-cyanomethyl-9-hydroxy-6,7,7a,8,9,10,11,11a-octahydro-5H-dibenzo[a,c]cycloheptene-3-carboxylic acid (2-methyl-pyridin-3-yl)-amide;
(7aR,9S,11aR)-11a-Benzyl-9-cyanomethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2,4-dimethyl-pyrimidin-5-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (3,5-dimethyl-pyrazin-2-yl)-amide; (7aS,9R,11aS)-11a-Benzyl-9-ethyl-9-hydroxy-6,7,7a,8,9,10,11,11a-octahydro-5H-dibenzo[a,c]cycloheptene-3-carboxylic acid (3-methyl-pyridin-4-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2,6-dimethyl-pyridin-3-yl)-amide;
(7aS,9R,11aS)-11a-Benzyl-9-ethyl-9-hydroxy-6,7,7a,8,9,10,11,11a-octahydro-5H-dibenzo[a,c a,c]cycloheptene-3-carboxylic acid (3-methyl-pyridin-2-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid [1,3,4]thiadiazol-2-ylamide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2-methyl-2H-pyrazol-3-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2,5-dimethyl-2H-pyrazol-3-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2,4-dimethyl-pyrimidin-5-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (1-methyl-1H-tetrazol-5-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (5-methyl-2H-pyrazol-3-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2-methyl-pyridin-3-ylmethyl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2-morpholin-4-yl-ethyl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (1-methyl-4-oxo-4,5-dihydro-1H-imidazol-2-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2-ethyl-2H-pyrazol-3-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid [2-methyl-6-(2H-pyrazol-3-yl)-pyridin-3-yl]-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid [2-methyl-6-(1H-pyrazol-4-yl)-pyridin-3-yl]-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid methyl-(2-methyl-pyridin-3-yl)-amide;
(7aS,9R,11aR)-11a-Ethyl-9-hydroxy-9-(2,2,2-trifluoro-ethoxymethyl)-6,7,7a,8,9,10,11,11a-octahydro-5H-dibenzo[a,c]cycloheptene-3-carboxylic acid (2-methyl-pyridin-3-yl)-amide: compound with (7aR,9S,11aS)-11a-ethyl-9-hydroxy-9-(2,2,2-trifluoro-ethoxymethyl)-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-ethoxymethyl-9-hydroxy-6,7,7a,8,9,10,11,11a-octahydro-5H-dibenzo[a,c]cycloheptene-3-carboxylic acid (2-methyl-pyridin-3-yl)-amide;
(7aR,9S,11aR)-11a-Benzyl-9-ethoxymethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-(2,2,2-trifluoro-ethoxymethyl)-6,7,7a,8,9,10,11,11a-octahydro-5H-dibenzo[a,c]cycloheptene-3-carboxylic acid (2-methyl-pyridin-3-yl)-amide; compound with (7aR,9S,11aR-11a-Benzyl-9-hydroxy-9-(2,2,2-trifluoro-ethoxymethyl)-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-(oxetan-3-ylmethoxymethyl)-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-Benzyl-9-hydroxy-9-(oxetan-3-ylmethoxymethyl)-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-isopropoxymethyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-Benzyl-9-hydroxy-9-isopropoxymethyl-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-propoxymethyl-6,7,7a,8,9,10,11,11a-octahydro-5H-dibenzo[a,c]cycloheptene-3-carboxylic acid (2-methyl-pyridin-3-yl)-amide;
(7aR,9S,11aR-11a-Benzyl-9-hydroxy-9-propoxymethyl-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-(2,2,2-trifluoro-1-methyl-ethoxymethyl)-5,7,7a,8,9,10,11,11a-octahydro-dibenzo[c,e]oxepine-3-carboxylic acid (2-methyl-pyridin-3-yl)-amide; compound with (7aR,9S,11aR)-11a-Benzyl-9-hydroxy-9-(2,2,2-trifluoro-1-methyl-ethoxymethyl)-5,7,7a,8,9,10,11,11a-octahydro-dibenzo[c,e]oxepine-3-carboxylic acid (2-methyl-pyridin-3-yl)-amide;
(7aS,9R,11aS)-11a-Benzyl-9-hydroxy-9-prop oxymethyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR-11a-Benzyl-9-hydroxy-9-prop oxymethyl-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-(tetrahydro-pyran-4-yloxymethyl)-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-Benzyl-9-hydroxy-9-(tetrahydro-pyran-4-yloxymethyl)-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-phenoxymethyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-Benzyl-9-hydroxy-9-phenoxymethyl-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-hydroxymethyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-Benzyl-9-hydroxy-9-hydroxymethyl-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-(2-methane sulfonyl-ethoxymethyl)-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-Benzyl-9-hydroxy-9-(2-methanesulfonyl-ethoxymethyl)-6,7,7a,8,9,10,11,11a-octahydro-5H-dibenzo[a,c]cycloheptene-3-carboxylic acid (2-methyl-pyridin-3-yl)-amide;
(4aS,11bS)-11b-Benzyl-6-methyl-N-(2-methylpyridin-3-yl)-3-oxo-2,3,4,4 a,5,6,7,11b-octahydro-1H-dibenzo[c,e]azepine-9-carboxamide;
(3S,4aS,11bS)-11b-benzyl-3-hydroxy-6-methyl-N-(2-methylpyridin-3-yl)-2,3,4,4a,5,6,7,11b-octahydro-1H-dibenzo[c,e]azepine-9-carboxamide;
(7aS,11aS)-11a-Benzyl-N-(2-methylpyridin-3-yl)-7,9-dioxo-5,7,7a,8,9,10,11,11a-octahydro dibenzo[c,e]oxepine-3-carboxamide;
(7aS,9R,11aS)-11a-Benzyl-9-ethyl-9-hydroxy-N-(2-methylpyridin-3-yl)-5,7,7a,8,9,10,11,11a-octahydrodibenzo[c,e]oxepine-3-carboxamide;
(7aS,9R,11aS)-11a-Benzyl-9-ethyl-9-hydroxy-N-(2-methylpyridin-3-yl)-5-oxo-5,7,7a,8,9,10,11,11a-octahydrodibenzo[c,e]oxepine-3-carboxamide;
(7aR,9R,11aS)-11a-Benzyl-9-ethyl-9-hydroxy-6-oxo-6,7,7a,8,9,10,11,11a-octahydro-5H-dibenzo[a,c]cycloheptene-3-carboxylic acid (2-methyl-pyridin-3-yl)-amide;
(7aS,9R,11aR)-11a-Ethyl-9-hydroxy-N-(2-methylpyridin-3-yl)-9-propyl-6,7,7a,8,9,10,11,11a-octahydrodibenzo[b,d]oxepine-3-carboxamide; compound with (7aR,9S,11aS)-11a-ethyl-9-hydroxy-N-(2-methylpyridin-3-yl)-9-propyl-6,7,7a,8,9,10,11,11a-octahydrodibenzo[b,d]oxepine-3-carboxamide;
(7aR,9S,11aS)-11a-ethyl-9-hydroxy-N-(2-methylpyridin-3-yl)-9-propyl-6,7,7a,8,9,10,11,11a-octahydrodibenzo[b,d]oxepine-3-carboxamide;
(7aS,9R,11aR)-11a-ethyl-9-hydroxy-N-(2-methylpyridin-3-yl)-9-propyl-6,7,7a,8,9,10,11,11a-octahydrodibenzo[b,d]oxepine-3-carboxamide;
(7aS,9R,11aR)-11a-Ethyl-9-hydroxy-9-phenyl-6,7,7a,8,9,10,11,11a-octahydro-5H-dibenzo[a,c]cycloheptene-3-carboxylic acid (2-methyl-pyridin-3-yl)-amide;
(7aR,9S,11aS)-11a-ethyl-9-hydroxy-9-phenyl-6,7,7a,8,9,10,11,11a-octahydro-5H-dibenzo[a,c]cycloheptene-3-carboxylic acid (2-methyl-pyridin-3-yl)-amide;
(7aS,9R,10R,11aR)-11a-Ethyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide;
(7aR,9S,10S,11aS)-11a-Ethyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2-amino-phenyl)-amide;
(3R,4aS,11bS)-9-(1H-benzoimidazol-2-yl)-11b-benzyl-3-ethyl-2,3,4,4 a,5,6,7,11b-octahydro-1H-dibenzo[a,c]cyclohepten-3-ol;
(7aS,9R,11aR)-11a-Ethyl-9-hydroxy-9-propyl-7a,8,9,10,11,11a-hexahydro-7H-dibenzo[a,c]cycloheptene-3-carboxylic acid (2-methyl-pyridin-3-yl)-amide compound with (7aR,9S,11aS)-11a-ethyl-9-hydroxy-9-propyl-7a,8,9,10,11,11a-hexahydro-7H-dibenzo[a,c]cycloheptene-3-carboxylic acid (2-methyl-pyridin-3-yl)-amide;
(7aS,9R,11aR)-11a-Ethyl-9-hydroxy-9-propyl-7a,8,9,10,11,11a-hexahydro-7H-dibenzo[a,c]cycloheptene-3-carboxylic acid (2-methyl-pyridin-3-yl)-amide;
(7aR,9S,11aS)-11a-ethyl-9-hydroxy-9-propyl-7a,8,9,10,11,11a-hexahydro-7H-dibenzo[a,c]cycloheptene-3-carboxylic acid (2-methyl-pyridin-3-yl)-amide;
(7aS,9R,11aS)-11a-Benzyl-9-ethyl-9-hydroxy-7a,8,9,10,11,11a-hexahydro-7H-dibenzo[a,c]cycloheptene-3-carboxylic acid (2-methyl-pyridin-3-yl)-amide;
(7aR,9S,11aR)-11a-Benzyl-9-ethyl-9-hydroxy-7a,8,9,10,11,11a-hexahydro-7H-dibenzo[a,c]cycloheptene-3-carboxylic acid (2-methyl-pyridin-3-yl)-amide;
(7aS,9S,11aR)-11a-Ethyl-9-hydroxy-9-(3,3,3-trifluoro-propyl)-6,7,7a,8,9,10,11,11a-octahydro-5H-dibenzo[a,c]cycloheptene-3-carboxylic acid (2-methyl-pyridin-3-yl)-amide; compound with (7aR,9R,11aS)-11a-ethyl-9-hydroxy-9-(3,3,3-trifluoro-propyl)-6,7,7a,8,9,10,11,11a-octahydro-5H-dibenzo[a,c]cycloheptene-3-carboxylic acid (2-methyl-pyridin-3-yl)-amide;
(7aS,9R,11aR)-11a-Ethyl-9-hydroxy-N-(2-methylpyridin-3-yl)-9-phenyl-6,7,7a,8,9,10,11,11a-octahydrodibenzo[b,d]oxepine-3-carboxamide; compound with (7aR,9S,11aS)-11a-ethyl-9-hydroxy-N-(2-methylpyridin-3-yl)-9-phenyl-6,7,7a,8,9,10,11,11a-octahydrodibenzo[b,d]oxepine-3-carboxamide;
(7aS,9R,10R,11aR)-11a-Ethyl-9,10-dihydroxy-N-(2-methylpyridin-3-yl)-9-phenyl-6,7,7a,8,9,10,11,11a-octahydro dibenzo[b,d]oxepine-3-carboxamide; compound with (7aR,9S,10S,11aS)-11a-ethyl-9,10-dihydroxy-N-(2-methylpyridin-3-yl)-9-phenyl-6,7,7a,8,9,10,11,11a-octahydrodibenzo[b,d]oxepine-3-carboxamide;
(7aS,9R,11aR)-11a-Ethyl-9-propyl-9-hydroxy-N-(2-methylpyridin-3-yl)-5,7,7a,8,9,10,11,11a-octahydro dibenzo[c,e]oxepine-3-carboxamide; compound with (7aR,9S,11aS)-11a-ethyl-9-propyl-9-hydroxy-N-(2-methylpyridin-3-yl)-5,7,7a,8,9,10,11,11a-octahydrodibenzo[c,e]oxepine-3-carboxamide;
(7aR,9S,11aS)-11a-ethyl-9-propyl-9-hydroxy-N-(2-methylpyridin-3-yl)-5,7,7a,8,9,10,11,11a-octahydrodibenzo[c,e]oxepine-3-carboxamide;
(7aS,9R,11aR)-11a-Ethyl-9-propyl-9-hydroxy-N-(2-methylpyridin-3-yl)-5,7,7a,8,9,10,11,11a-octahydrodibenzo[c,e]oxepine-3-carboxamide;
(7aS,9S,11aR)-11a-Ethyl-9-hydroxy-9-isobutyl-5,7,7a,8,9,10,11,11a-octahydro-dibenzo[c,e]oxepine-3-carboxylic acid (2-methyl-pyridin-3-yl)-amide;
(7aS,9R,11aR)-9,11a-diethyl-9-hydroxy-N-(2-methylpyridin-3-yl)-5,7,7a,8,9,10,11,11a-octahydrodibenzo[c,e]oxepine-3-carboxamide;
(7aR,9S,11aS)-9,11a-diethyl-9-hydroxy-N-(2-methylpyridin-3-yl)-5,7,7a,8,9,10,11,11a-octahydrodibenzo[c,e]oxepine-3-carboxamide;
(7aR,9S,11aS)-9,11a-diethyl-9-hydroxy-N-(2-methylpyridin-3-yl)-5,7,7a,8,9,10,11,11a-octahydrodibenzo[c,e]oxepine-3-carboxamide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (4-amino-phenyl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (3-amino-phenyl)-amide;
(7aS,9R,11aS)-11a-Benzyl-9-ethyl-9-hydroxy-6,7,7a,8,9,10,11,11a-octahydro-5H-benzo[c]pyrrolo[1,2-a]azepine-2-carboxylic acid (2-methyl-pyridin-3-yl)-amide; compound with (7aR,9S,11aR)-11a-benzyl-9-ethyl-9-hydroxy-6,7,7a,8,9,10,11,11a-octahydro-5H-benzo[c]pyrrolo[1,2-a]azepine-2-carboxylic acid (2-methyl-pyridin-3-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2-amino-phenyl)-amide; compound with (7aR,9S,11aR)-11a-benzyl-9-ethyl-9-hydroxy-6,7,7a,8,9,10,11,11a-octahydro-5H-dibenzo[a,c]cycloheptene-3-carboxylic acid (2-amino-phenyl)-amide;
(3R,4aS,11bS)-9-(1H-Benzoimidazol-2-yl)-11b-benzyl-3-ethyl-2,3,4,4a,5,6,7,11b-octahydro-1H-dibenzo[a,c]cyclohepten-3-ol; compound with (3S,4aR,11bR)-9-(1H-benzoimidazol-2-yl)-11b-benzyl-3-ethyl-2,3,4,4a,5,6,7,11b-octahydro-1H-dibenzo[a,c]cyclohepten-3-ol;
(3R,4aS,11bS)-9-(1H-Benzoimidazol-2-yl)-11b-benzyl-3-ethyl-2,3,4,4a,5,6,7,11b-octahydro-1H-dibenzo[a,c]cyclohepten-3-ol;
(3S,4aR,11bR)-9-(1H-benzoimidazol-2-yl)-11b-benzyl-3-ethyl-2,3,4,4a,5,6,7,11b-octahydro-1H-dibenzo[a,c]cyclohepten-3-ol;
(7aS,9R,11aR)-11a-Ethyl-9-hydroxy-9-propyl-6,7,7a,8,9,10,11,11a-octahydro-5H-dibenzo[a,c]cycloheptene-3-carboxylic acid (2-amino-pyridin-3-yl)-amide; compound with (7aR,9S,11aS)-11a-Ethyl-9-hydroxy-9-propyl-6,7,7a,8,9,10,11,11a-octahydro-5H-dibenzo[a,c]cycloheptene-3-carboxylic acid (2-amino-pyridin-3-yl)-amide;
(7aS,9R,11aS)-11a-Cyclopropylmethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-cyclopropylmethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide; or
(7aS,9R,11aR)-11a-Ethyl-9-hydroxy-9-propyl-6,7,7a,8,9,10,11,11a-octahydro-5H-dibenzo[a,c]cycloheptene-3-carboxylic acid (2-chloro-phenyl)-amide.

In a fourteenth embodiment the invention provides a compound according to the first embodiment wherein the compound is of Formula (I)c or Formula (I)d

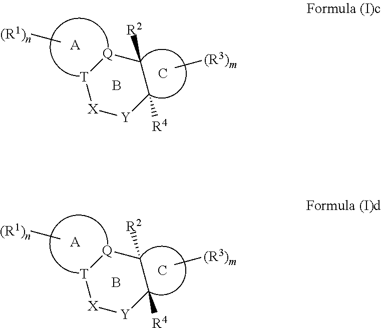

In a fifteenth embodiment the invention provides a compound according to the fourteenth embodiment wherein Ring A is optionally substituted phenyl, optionally substituted pyrazolyl or optionally substituted pyrrolyl.

In a sixteenth embodiment the invention provides a compound according to any of the foregoing embodiments wherein Ring C is optionally substituted cyclohexyl or optionally substituted cyclohexenyl.

In a seventeenth embodiment the invention provides a compound according to any of the foregoing embodiments wherein X is —C(R⁵)₂—, —C(R⁵)—, —C(═O)—, —O— or —N(R^{a})—.

In an eighteenth embodiment the invention provides a compound according to any of the foregoing embodiments wherein Y is —C(R⁵)₂C(R⁵)₂—, —C(R⁵)C(R⁵)₂—, —C(R⁵)₂C(R⁵)—, —OC(R⁵)₂—, —N(R^{a})C(R⁵)₂—, —C(R⁵)₂N(R^{a})—, —C(═O)C(R⁵)₂—, —C(R⁵)₂C(═O)—, —O—C(═O)—, —C(═O)—O—, —C(R⁵)₂—O—, —O—C(R⁵)₂— or —O—C(R⁵)(R^{b}).

In a nineteenth embodiment the invention provides a compound according to any of the foregoing embodiments wherein R¹ is —COOR^{a}, OR^{a}, —O-optionally substituted (C₁-C₃)alkylene-optionally substituted phenyl, —O-optionally substituted (C₁-C₃)alkylene-optionally substituted pyridinyl, optionally substituted (C₁-C₃)alkyl, —C(O)N(R^{a})(CH₂)ᵣ—R^{b}, or —N(R^{a})C(O)(CH₂)ᵣ—R^{b}.

In a twentieth embodiment the invention provides a compound according to any of the foregoing embodiments wherein R² is —(CH₂)ᵣ-optionally substituted phenyl, -optionally substituted (C₃-C₆)cycloalkyl, optionally substituted (C₁-C₃)alkyl, or —(CH₂)ᵣ-optionally substituted heteroaryl.

In a twenty-first embodiment the invention provides a compound according to any of the foregoing embodiments wherein R³ is independently H, —CF₃, optionally substituted (C₂-C₆)alkynyl, oxo, —OR′, —OP(═O)(OH)(OH), optionally substituted (C₁-C₄)alkyl, —CH₂-optionally substituted cyclopropyl, or optionally substituted phenyl.

In a twenty-second embodiment the invention provides a compound according to any of the foregoing embodiments wherein R^{b} is optionally substituted phenyl, -optionally substituted pyrimidinyl, optionally substituted pyridinyl, optionally substituted pyrazolyl or optionally substituted tetrazolyl.

In a twenty-third embodiment the invention provides a compound according to any of the foregoing embodiments wherein Q is C.

In a twenty-fourth embodiment the invention provides a compound according to any of the foregoing embodiments wherein T is C.

In a twenty-fifth embodiment the invention provides a compound of according to any of the foregoing embodiments wherein the compound is
(4aS,11bS)-11b-Benzyl-9-hydroxy-1,2,4,4a,5,6,7,11b-octahydro-dibenzo[a,c]cyclohepten-3-one; compound with (4aR,11bR)-11b-benzyl-9-hydroxy-1,2,4,4a,5,6,7,11b-octahydro-dibenzo[a,c]cyclohepten-3-one;
(7aR,11aS)-11a-Benzyl-9-oxo-6,7,7a,8,9,10,11,11a-octahydro-5H-dibenzo[a,c]cycloheptene-3-carboxylic acid (2-methyl-pyridin-3-yl)-amide; compound with (7aS,11aR)-11a-benzyl-9-oxo-6,7,7a,8,9,10,11,11a-octahydro-5H-dibenzo[a,c]cycloheptene-3-carboxylic acid (2-methyl-pyridin-3-yl)-amide;
(7aR,9R,11aS)-11a-Benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide; compound with (7aS,9S,11aR)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide;
(7aR,9S,11aS)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide; compound with (7aS,9R,11aR)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-N-(2-methylpyridin-3-yl)-6-oxo-9-(trifluoromethyl)-6,7,7a,8,9,10,11,11a-octahydro-5H-dibenzo[b,d]azepine-3-carboxamide;
(7aR,9R,11aS)-11a-Benzyl-9-hydroxy-9-trifluoromethyl-6,7,7a,8,9,10,11,11a-octahydro-5H-dibenzo[a,c]cycloheptene-3-carboxylic acid (2-methyl-2H-pyrazol-3-yl)-amide;
(7aR,9R,11aS)-11a-Benzyl-9-hydroxy-9-trifluoromethyl-6,7,7a,8,9,10,11,11a-octahydro-5H-dibenzo[a,c]cycloheptene-3-carboxylic acid (3-methyl-pyridin-4-yl)-amide;
(7aS,9S,11aR)-11a-Benzyl-9-hydroxy-9-trifluoromethyl-6,7,7a,8,9,10,11,11a-octahydro-5H-dibenzo[a,c]cycloheptene-3-carboxylic acid (2-methyl-2H-pyrazol-3-yl)-amide;
(7aR,9R,11aS)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide;
(7aS,9R,11aR)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide;
(7aR,9R,11aS)-11a-Benzyl-9-hydroxy-9-trifluoromethyl-6,7,7a,8,9,10,11,11a-octahydro-5H-dibenzo[a,c]cycloheptene-3-carboxylic acid (2-methyl-pyridin-3-yl)-amide;
(7aS,9S,11aR)-11a-Benzyl-9-hydroxy-9-trifluoromethyl-6,7,7a,8,9,10,11,11a-octahydro-5H-dibenzo[a,c]cycloheptene-3-carboxylic acid (2-methyl-pyridin-3-yl)-amide;
(3R,4aR,11bS)-11b-Benzyl-3-ethyl-3-hydroxy-6-methyl-N-(2-methylpyridin-3-yl)-7-oxo-2,3,4,4a,5,6,7,11b-octahydro-1H-dibenzo[c,e]azepine-9-carb oxamide;
(7aR,9R,11aS)-11a-Benzyl-9-hydroxy-N-(2-methylpyridin-3-yl)-5-oxo-9-(trifluoromethyl)-5,7,7a,8,9,10,11,11a-octahydrodibenzo[c,e]oxepine-3-carboxamide;
(7aR,9R,11aS)-11a-Benzyl-9-hydroxy-N-(2-methylpyridin-3-yl)-9-(trifluoromethyl)-5,7,7a,8,9,10,11,11a-octahydro dibenzo[c,e]oxepine-3-carb oxamide;
(3R,4aR,11bS)-11b-Benzyl-3-hydroxy-6-methyl-N-(2-methylpyridin-3-yl)-3-(trifluoromethyl)-2,3,4,4a,5,6,7,11b-octahydro-1H-dibenzo[c,e]azepine-9-carboxamide; or
(7aR,9R,11aS)-11a-Benzyl-9-hydroxy-5-oxo-9-trifluoromethyl-6,7,7a,8,9,10,11,11a-octahydro-5H-dibenzo[a,c]cycloheptene-3-carboxylic acid (2-methyl-pyridin-3-yl)-amide.

In a twenty-sixth embodiment the invention provides the compound
11b-Benzyl-9-methoxy-1,2,5,6,7,11b-hexahydro-dibenzo[a,c]cyclohepten-3-one;
11b-Benzyl-9-hydroxy-1,2,5,6,7,11b-hexahydro-dibenzo[a,c]cyclohepten-3-one;
(9R,11aS)-11a-Benzyl-9-hydroxy-9-methyl-6,7,9,10,11,11a-hexahydro-5H-dibenzo[a,c]cycloheptene-3-carboxylic acid (2-methyl-pyridin-3-yl)-amide; compound with
(9S,11aR)-11a-benzyl-9-hydroxy-9-methyl-6,7,9,10,11,11a-hexahydro-5H-dibenzo[a,c]cycloheptene-3-carboxylic acid (2-methyl-pyridin-3-yl)-amide; (A-1292844.0) or
(4aS,9aS)-4a-Benzyl-octahydro-benzocycloheptene-2,5-dione; compound with (4aR,9aR)-4a-benzyl-octahydro-benzocycloheptene-2,5-dione.

In a twenty-seventh embodiment the invention provides a pharmaceutical composition comprising a compound of Formula (I) and a pharmaceutically acceptable carrier or excipient.

In a twenty-eighth embodiment the invention provides a method of treating a disease or condition comprising administering a therapeutically effective amount of a compound of Formula (I).

In a twenty-ninth embodiment the invention provides a method according to the twenty-eighth embodiment wherein the disease or condition to be treated is acquired immunodeficiency syndrome (AIDS), acute adrenal insufficiency, addiction, Addison's Disease, adrenal function, allergic rhinitis, allergies, Alzheimer's, anorexia, angioneurotic edema, ankylosing spondylitis, anxiety, asthma, auto-immunity, autoimmune chronic active hepatitis, autoimmune diseases, blepharitis, bursitis, cachexia, cardiovascular disease, cerebral edema, choroidal neovascularization due to age-related macular degeneration, chronic kidney disease, chronic obstructive pulmonary disease, chronic primary adrenal insufficiency, chronic retinal detachment, compulsive behavior, congenital adrenal hyperplasia, cognitive dysfunction, conjunctivitis, cirrhosis, Crohn's disease, Cushing's syndrome, depression, diabetes, diabetes mellitus, diabetic microangiopathy, diabetic neuropathy, diabetic retinopathy, dry eye syndrome, frailty, giant cell arteritis, glaucoma, granulomatous polyarteritis, hay fever, hepatitis, HPA axis suppression and regulation, human immunodeficiency virus (HIV), hypercalcemia, hypercortisolemia, hypergylcemia, hypertension, immune proliferation/apoptosis, immunodeficiency, immunomodulation, inflammation, inflammation of the eye, inflammatory bowel disease, inhibition of myeloid cell lines, insulin dependent diabetes mellitus, insulin-dependent diabetes mellitus glaucoma, insulin resistance, iridocyclitis, juvenile idiopathic arthritis, juvenile rheumatoid arthritis, leukemia, Little's syndrome, lupus, lymphoma, macular degeneration, macular edema, a malignancy, medical catabolism, multi-drug resistance, multiple sclerosis, neurodgeneration, obesity, ocular or macular edema, ocular neovascular disease, organ transplantation, modulation of the Th1/Th2 cytokine balance, optic neuritis, optic pits, neuropathy, osteoarthritis, osteoporosis, Parkinson's, plaque psoriasis, polyarteritis nodosa, post-laser treatment complications, post-surgical bone fracture, post-traumatic stress syndrome, prevention of muscle frailty, psoriasis, psoriatic arthritis, psychosis, regulation of carbohydrate, protein and lipid metabolism, regulation of electrolyte and water balance, regulation of functions of the cardiovascular, kidney, central nervous, immune, or skeletal muscle systems, retinopathy of prematurity, rheumatic fever, rheumatoid arthritis, rhinitis, scleritis, secondary adrenal insufficiency, stroke and spinal cord injury, sympathetic ophthalmia, systemic lupus erythematosus, Syndrome X, tendonitis, thrombocytopenia, tissue rejection, ulcerative colitis, urticaria, uveitis, viral infection, Wegener's granulomatosis or wound healing.

In a twenty-ninth embodiment the invention provides the use of a compound of Formula (I) as a medicament.

In a thirtieth embodiment the invention provides the use of a compound of Formula (I) as a medicament wherein the use is according to the twenty-eight embodiment.

In a thirty-first embodiment the invention provides a kit comprising a compound or pharmaceutical composition according to any of the foregoing embodiments.

In a thirty-second embodiment the invention provides a kit according to the twenty-ninth embodiment further comprising instructions for use.

In a thirty-third embodiment the invention provides a process for the preparation of a compound of Formula 2

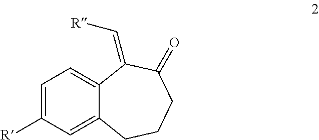

comprising the step of reacting compound of Formula 1

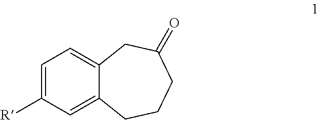

with a base until the reaction is substantially complete, then reacting the anion with acetaldehyde to form a compound of Formula 2

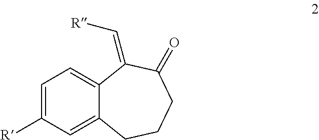

wherein
R′ is alkoxy and
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl;
   wherein r is independently 0, 1 or 2.

In a thirty-fourth embodiment the invention provides a process according to claim 30, further comprising the step of warming.

In a thirty-fifth embodiment the invention provdes a process for the preparation of a compound of Formula 3

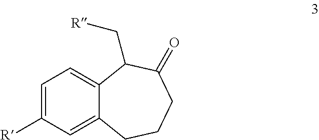

comprising the step of reacting compound of Formula 2

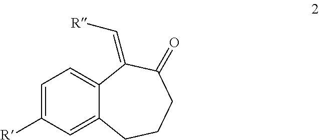

with a catalyst and hydrogen until the reaction is substantially complete to form a compound of Formula 3

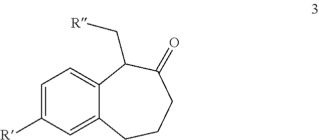

wherein
R′ is alkoxy and
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl;
   wherein r is independently 0, 1 or 2.

In a thirty-sixth embodiment the invention provides a process for the preparation of a compound of Formula 4

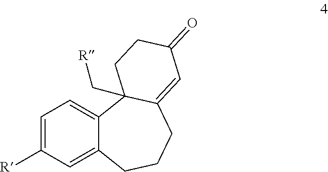

comprising the step of reacting compound of Formula 3

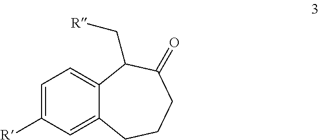

with a ketone and a base until the reaction is substantially complete to form a compound of Formula 4

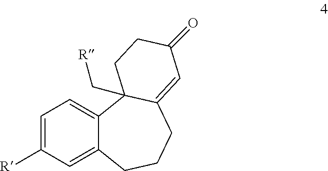

wherein
R′ is alkoxy and
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl.

In a thirty-seventh embodiment the invention provides a process for the preparation of a compound of Formula 6

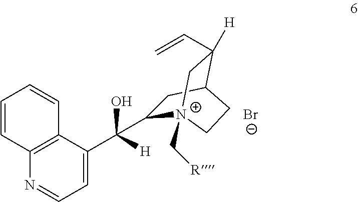

comprising the step of reacting compound of Formula 5

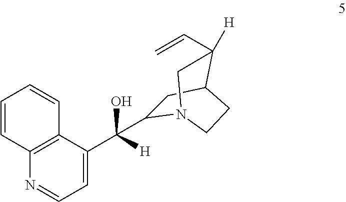

with 1-(bromomethyl)-2-fluoro-4-(trifluoromethyl)benzene until the reaction is substantially complete to form a compound of Formula 6

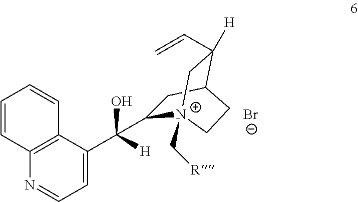

wherein R″″ is arylhalide.

In a thirty-eighth embodiment the invention provides a process for the preparation of compounds of Formulas 3a and 3b

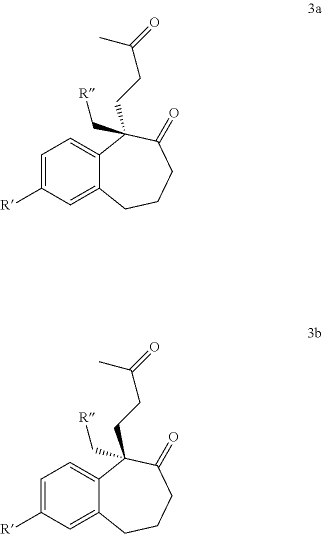

comprising reacting a compound of Formula 3

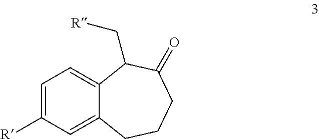

with an eneone, a base and a compound of Formula 6

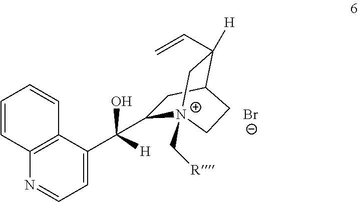

until the reaction is substantially complete to form compounds of Formulas 3a and 3b

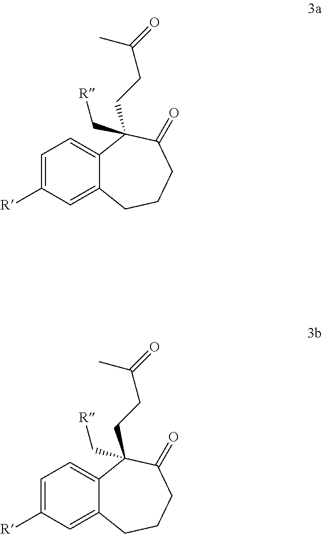

wherein
R′ is alkoxy;
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl; and
R″″ is arylhalide.

In a thirty-ninth embodiment the invention provides a process for preparing compounds of Formula 3c and 3d

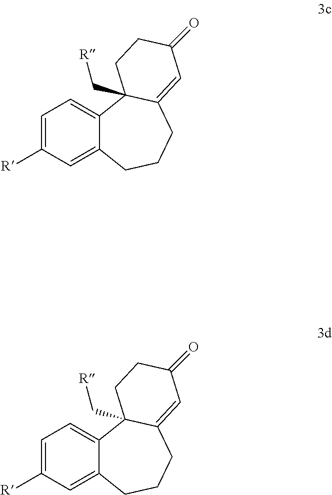

comprising reacting compounds of Formulas 3a and 3b

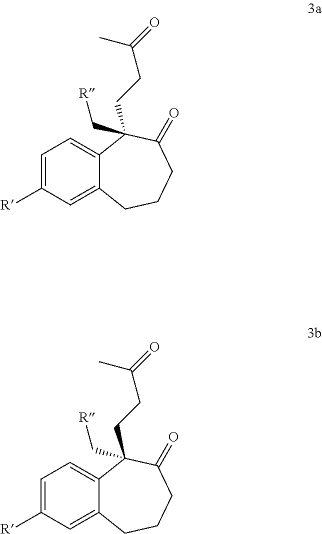

with a base until the reaction is substantially complete to form a compound of Formulas 3c and 3d

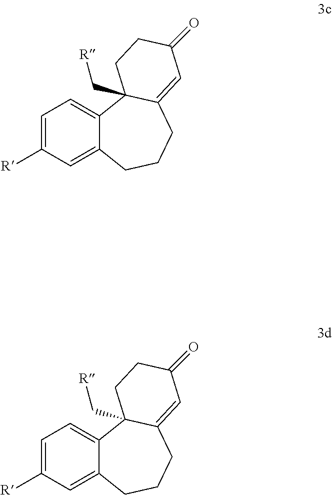

wherein
R′ is alkoxy and
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl.

In a fortieth embodiment the invention provides a process for preparing a compound of Formula 4a

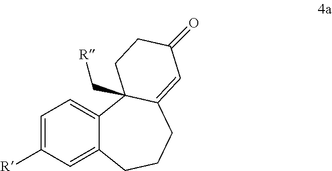

comprising fractional crystallization of formulas 3c and 3d

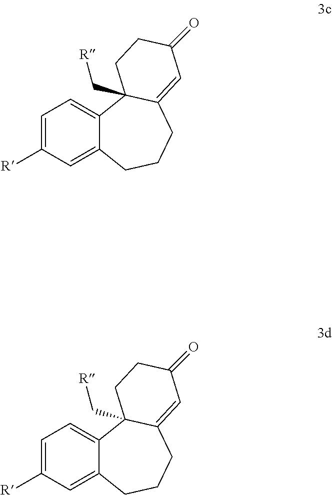

until the reaction is substantially complete to form a compound of Formula 4a

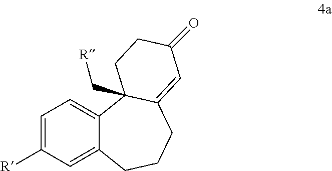

wherein
R′ is alkoxy and
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl.

In a forty-first embodiment the invention provides a process for preparing a compound of Formula 7

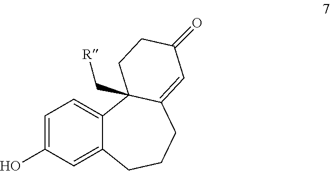

comprising reacting a compound of Formula 4a

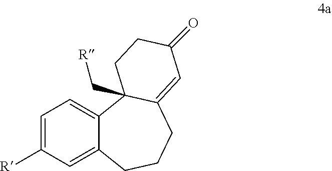

with an acid and methionine until the reaction is substantially complete to form a compound of Formula 7

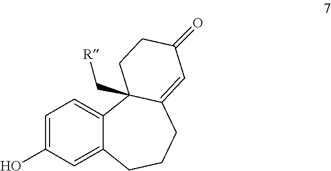

wherein
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl.

In a forty-second embodiment the invention provides the process according to the forty-first embodiment, wherein the acid is methanesulfonic acid.

In a forty-third embodiment the invention provides a process for preparing a compound of Formula 8

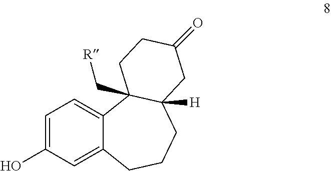

comprising reacting a compound of Formula 7

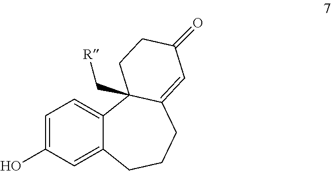

with hydrogen and a catalyst until the reaction is substantially complete to form a compound of Formula 8

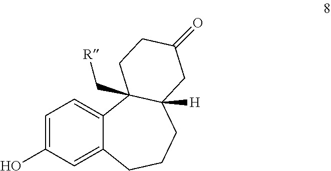

wherein
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl.

In a forty-fourth embodiment the invention provides a process for preparing a compound of Formula 9

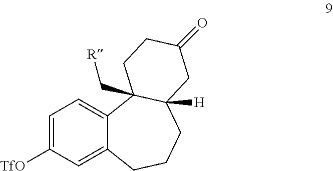

comprising reacting a compound of Formula 8

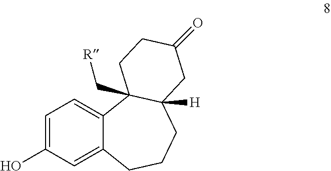

with a triflating reagent N-phenylbis(trifluoromethanesulfonimide and a base until the reaction is substantially complete to form a compound of Formula 9

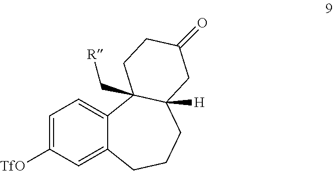

wherein
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl.

In a forty-fifth embodiment the invention provides the process according to forty-fourth embodiment wherein the triflating reagent is N-phenylbis(trifluoromethanesulfonimide.

In a forty-sixth embodiment the invention provides a process for preparing a compound of Formula 10

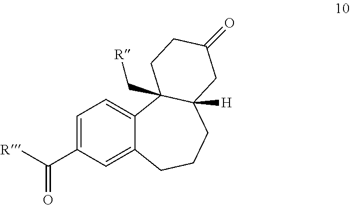

comprising reacting a compound of Formula 9

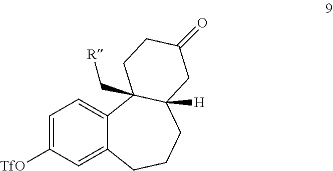

with carbon monoxide and a catalyst until the reaction is substantially complete to form a compound of Formula 10

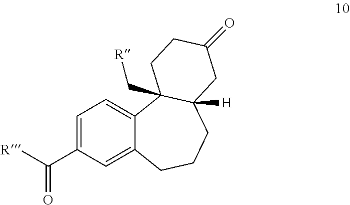

wherein
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl and
R′″ is optionally substituted aminoaryl, optionally substituted aminoheterocyclyl, optionally substituted aminoheteroaryl or optionally substituted aminocycloalkyl.

In a forty-seventh embodiment the inventi provides a process for preparing a compound of Formula 11

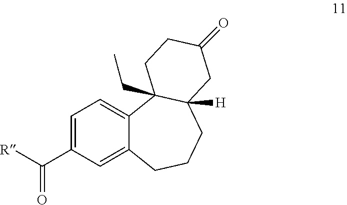

comprising reacting a compound of Formula 10

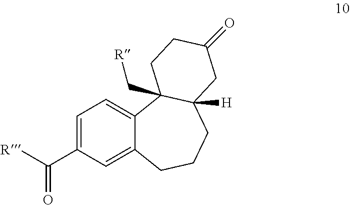

with a base until the reaction is substantially complete, then coupling to an amine to form a compound of Formula 11

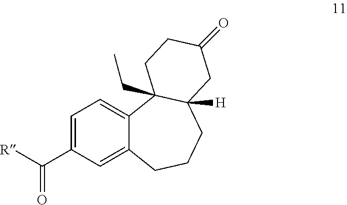

wherein
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl and
R′″ is optionally substituted aminoaryl, optionally substituted aminoheterocyclyl, optionally substituted aminoheteroaryl or optionally substituted aminocycloalkyl.

In a forty-eighth embodiment the invention provides a process for preparing a compound of Formula 12

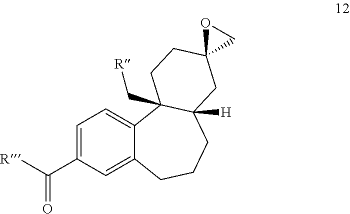

comprising reacting a compound of Formula 11

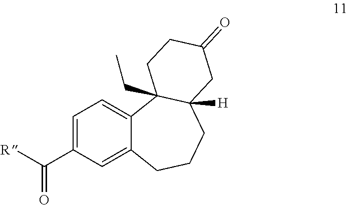

with a base and trimethylsulfoxonium halide until the reaction is substantially complete to form a compound of Formula 12

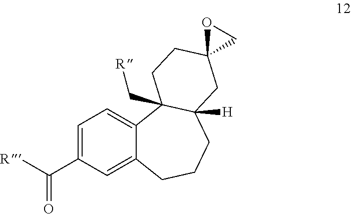

wherein
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl and
R′″ is optionally substituted aminoaryl, optionally substituted aminoheterocyclyl, optionally substituted aminoheteroaryl or optionally substituted aminocycloalkyl.

In a forty-ninth embodiment the invention provides a process for preparing a compound of Formula 13

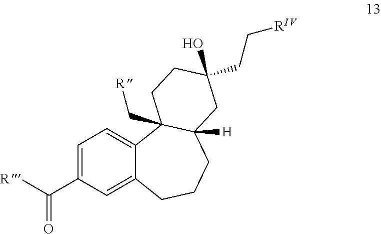

comprising reacting a compound of Formula 12

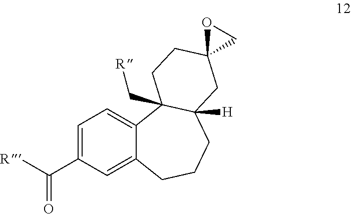

with a metal halide until the reaction is substantially complete to form a compound of Formula 13

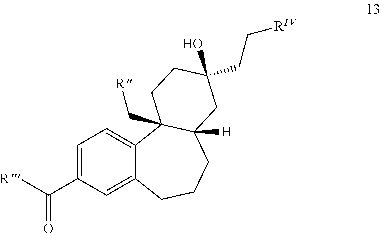

wherein
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl;
R′″ is optionally substituted aminoaryl, optionally substituted aminoheterocyclyl, optionally substituted aminoheteroaryl or optionally substituted aminocycloalkyl and
R^{IV} is H, optionally substituted (C₁-C₃)alkyl, OH or —O-optionally substituted (C₁-C₃)alkyl.

### DETAILED DESCRIPTION OF THE INVENTION

The glucocorticoid receptor (GR) is present in glucocorticoid responsive cells where it resides in the cytosol in an inactive state until it is stimulated by an agonist. Upon stimulation the glucocorticoid receptor translocates to the cell nucleus where it specifically interacts with DNA and/or protein(s) and regulates transcription in a glucocorticoid responsive manner. Two examples of proteins that interact with the glucocorticoid receptor are the transcription factors, API and NFK-B. Such interactions result in inhibition of API- and NFK-B-mediated transcription and are believed to be responsible for some of the anti-inflammatory activity of endogenously administered glucocorticoids. In addition, glucocorticoids may also exert physiologic effects independent of nuclear transcription. Biologically relevant glucocorticoid receptor agonists include Cortisol and corticosterone. Many synthetic glucocorticoid receptor agonists exist including dexamethasone, prednisone and prednisilone. By definition, glucocorticoid receptor antagonists bind to the receptor and prevent glucocorticoid receptor agonists from binding and eliciting GR mediated events, including transcription. RU486 is an example of a non-selective glucocorticoid receptor antagonist.

Although there are glucocorticoid receptor therapies in the art, there is a continuing need for and a continuing search in this field of art for selective glucocorticoid receptor therapies. Thus, the identification of non-steroidal compounds which have specificity for one or more steroid receptors, but which have reduced or no cross-reactivity for other steroid or intracellular receptors, is of significant value in this field.

Many autoimmune diseases and disease associated with chronic inflammation, as well as acute responses, have been linked to excessive or unregulated production or activity of one or more cytokines.

The compounds of the invention are also useful in the treatment of rheumatoid arthritis, ankylosing spondilitis, a solid tumor, a sarcoma, fibrosarcoma, osteoma, melanoma, retinoblastoma, an ocular disease, a cancer, a rhabdomyosarcoma, glioblastoma, neuroblastoma, teratocarcinoma, hypersensitivity reactions, hyperkinetic movement disorders, hypersensitivity pneumonitis, hypertension, hypokinetic movement disorders, aordic and peripheral aneuryisms, hypothalamic-pituitary-adrenal axis evaluation, aortic dissection, arterial hypertension, arteriosclerosis, arteriovenous fistula, ataxia, spinocerebellar degenerations, streptococcal myositis, structural lesions of the cerebellum, subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, telangiectasia, thromboangitis obliterans, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, heart transplant rejection, hemachromatosis, hemodialysis, hemolytic uremic syndrome/thrombolytic thrombocytopenic purpura, hemorrhage, idiopathic pulmonary fibrosis, antibody mediated cytotoxicity, Asthenia, infantile spinal muscular atrophy, inflammation of the aorta, influenza A, ionizing radiation exposure, iridocyclitis/uveitis/optic neuritis, juvenile spinal muscular atrophy, lymphoma, myeloma, leukaemia, malignant ascites, hematopoietic cancers, a diabetic condition such as insulin-dependent diabetes mellitus glaucoma, diabetic retinopathy or microangiopathy, sickle cell anaemia, chronic inflammation, glomerulonephritis, graft rejection, Lyme disease, von Hippel Lindau disease, pemphigoid, Paget's disease, fibrosis, sarcoidosis, cirrhosis, thyroiditis, hyperviscosity syndrome, Osler-Weber-Rendu disease, chronic occlusive pulmonary disease, asthma or edema following burns, trauma, radiation, stroke, hypoxia, ischemia, ovarian hyperstimulation syndrome, post perfusion syndrome, post pump syndrome, post-MI cardiotomy syndrome, preeclampsia, menometrorrhagia, endometriosis, pulmonary hypertension, infantile hemangioma, or infection by Herpes simplex, Herpes Zoster, human immunodeficiency virus, parapoxvirus, protozoa or toxoplasmosis, progressive supranucleo palsy, primary pulmonary hypertension, radiation therapy, Raynaud's phenomenon, Raynaud's disease, Refsum's disease, regular narrow QRS tachycardia, renovascular hypertension, restrictive cardiomyopathy, sarcoma, senile chorea, senile dementia of Lewy body type, shock, skin allograft, skin changes syndrome, ocular or macular edema, ocular neovascular disease, scleritis, radial keratotomy, uveitis, vitritis, myopia, optic pits, chronic retinal detachment, post-laser treatment complications, conjunctivitis, Stargardt's disease, Eales disease, retinopathy, macular degeneration, restenosis, ischemia/reperfusion injury, ischemic stroke, vascular occlusion, carotid obstructive disease, ulcerative colitis, inflammatory bowel disease, diabetes, diabetes mellitus, insulin dependent diabetes mellitus, allergic diseases, dermatitis scleroderma, graft versus host disease, organ transplant rejection (including but not limited to bone marrow and solid organ rejection), acute or chronic immune disease associated with organ transplantation, sarcoidosis, disseminated intravascular coagulation, Kawasaki's disease, nephrotic syndrome, chronic fatigue syndrome, Wegener's granulomatosis, Henoch-Schoenlein purpurea, microscopic vasculitis of the kidneys, chronic active hepatitis, septic shock, toxic shock syndrome, sepsis syndrome, cachexia, infectious diseases, parasitic diseases, acquired immunodeficiency syndrome, acute transverse myelitis, Huntington's chorea, stroke, primary biliary cirrhosis, hemolytic anemia, malignancies, Addison's disease, idiopathic Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, peripheral vascular disorders, peritonitis, pernicious anemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis A, Hepatitis B, Hepatitis C, His bundle arrythmias, HIV infection/HIV neuropathy, common varied immunodeficiency (common variable hypogammaglobulinaemia), dilated cardiomyopathy, female infertility, ovarian failure, premature ovarian failure, fibrotic lung disease, chronic wound healing, cryptogenic fibrosing alveolitis, post-inflammatory interstitial lung disease, interstitial pneumonitis, pneumocystis carinii pneumonia, pneumonia, connective tissue disease associated interstitial lung disease, mixed connective tissue disease, associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthritis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, acute and chronic pain (different forms of pain), Goodpasture's syndrome, pulmonary manifestation of polyarteritis nodosa, acute rheumatic fever, rheumatoid spondylitis, Still's disease, systemic sclerosis, Sjögren's syndrome, Takayasu's disease/arteritis, autoimmune thrombocytopaenia, toxicity, transplants, and diseases involving inappropriate vascularization for example diabetic retinopathy, retinopathy of prematurity, choroidal neovascularization due to age-related macular degeneration, and infantile hemangiomas in human beings. In addition, such compounds may be useful in the treatment of disorders such as ascites, effusions, and exudates, including for example macular edema, cerebral edema, acute lung injury, adult respiratory distress syndrome (ARDS), proliferative disorders such as restenosis, fibrotic disorders such as hepatic cirrhosis and atherosclerosis, mesangial cell proliferative disorders such as diabetic nephropathy, malignant nephrosclerosis, thrombotic microangiopathy syndromes, and glomerulopathies, myocardial angiogenesis, coronary and cerebral collaterals, ischemic limb angiogenesis, ischemia/reperfusion injury, peptic ulcer Helicobacter related diseases, virally-induced angiogenic disorders, preeclampsia, menometrorrhagia, cat scratch fever, rubeosis, neovascular glaucoma and retinopathies such as those associated with diabetic retinopathy, retinopathy of prematurity, or age-related macular degeneration. In addition, these compounds can be used as active agents against hyperproliferative disorders such as thyroid hyperplasia (especially Grave's disease), and cysts (such as hypervascularity of ovarian stroma characteristic of polycystic ovarian syndrome (Stein-Leventhal syndrome) and polycystic kidney disease since such diseases require a proliferation of blood vessel cells for growth and/or metastasis.

Compounds of Formula (I) of the invention can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the compound of the present invention. The additional agent also can be an agent that imparts a beneficial attribute to the therapeutic composition e.g., an agent that affects the viscosity of the composition.

It should further be understood that the combinations which are to be included within this invention are those combinations useful for their intended purpose. The agents set forth below are illustrative for purposes and not intended to be limited. The combinations, which are part of this invention, can be the compounds of the present invention and at least one additional agent selected from the lists below. The combination can also include more than one additional agent, e.g., two or three additional agents if the combination is such that the formed composition can perform its intended function.

Preferred combinations are non-steroidal anti-inflammatory drug(s) also referred to as NSAIDS which include drugs like ibuprofen. Other preferred combinations are corticosteroids including prednisolone; the well known side-effects of steroid use can be reduced or even eliminated by tapering the steroid dose required when treating patients in combination with the compounds of this invention. Non-limiting examples of therapeutic agents for rheumatoid arthritis with which a compound of Formula (I) of the invention can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-12, IL-15, IL-16, IL-21, IL-23, interferons, EMAP-II, GM-CSF, FGF, and PDGF. Compounds of the invention can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

Preferred combinations of therapeutic agents may interfere at different points in the autoimmune and subsequent inflammatory cascade; preferred examples include TNF antagonists like chimeric, humanized or human TNF antibodies, D2E7 (U.S. Pat. No. 6,090,382, HUMIRA™), CA2 (REMICADE™), SIMPONI™ (golimumab), CIMZIA™, ACTEMRA™, CDP 571, and soluble p55 or p75 TNF receptors, derivatives, thereof, p75TNFR1gG (ENBREL™) or p55TNFR1gG (Lenercept), and also TNFα converting enzyme (TACE) inhibitors; similarly IL-1 inhibitors (Interleukin-1-converting enzyme inhibitors, IL-1RA etc.) may be effective for the same reason. Other preferred combinations include Interleukin 11. Yet other preferred combinations are the other key players of the autoimmune response which may act parallel to, dependent on or in concert with IL-18 function; especially preferred are IL-12 antagonists including IL-12 antibodies or soluble IL-12 receptors, or IL-12 binding proteins. It has been shown that IL-12 and IL-18 have overlapping but distinct functions and a combination of antagonists to both may be most effective. Yet another preferred combination is non-depleting anti-CD4 inhibitors. Yet other preferred combinations include antagonists of the co-stimulatory pathway CD80 (B7.1) or CD86 (B7.2) including antibodies, soluble receptors or antagonistic ligands.

A compound of Formula (I) of the invention may also be combined with agents, such as methotrexate, 6-mercaptopurine, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNFα or IL-1 (e.g., NIK, IKK, p38 or MAP kinase inhibitors), IL-1β converting enzyme inhibitors, T-cell signaling inhibitors such as kinase inhibitors, metalloproteinase inhibitors, sulfasalazine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™) and p55TNFRIgG (Lenercept), sIL-1RI, sIL-1RII, sIL-6R), antiinflammatory cytokines (e.g. IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol HCl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline HCl, sulfadiazine, oxycodone HCl/acetaminophen, olopatadine HCl misoprostol, naproxen sodium, omeprazole, cyclophosphamide, rituximab, IL-1 TRAP, MRA, CTLA4-IG, IL-18 BP, anti-IL-12, Anti-IL15, BIRB-796, SCIO-469, VX-702, AMG-548, VX-740, Roflumilast, IC-485, CDC-801, S1P1 agonists (such as Fingolimod), and Mesopram. Preferred combinations include methotrexate or leflunomide and in moderate or severe rheumatoid arthritis cases, cyclosporin and anti-TNF antibodies as noted above.

Non-limiting examples of therapeutic agents for inflammatory bowel disease with which a compound of Formula (I) of the invention can be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; antibodies to or antagonists of other human cytokines or growth factors, for example, TNF, LT, IL-1, IL-2, IL-6, IL-7, IL-8, IL-12, IL-15, IL-16, IL-23, EMAP-II, GM-CSF, FGF, and PDGF; cell surface molecules such as CD2, CD3, CD4, CD8, CD25, CD28, CD30, CD40, CD45, CD69, CD90 or their ligands; methotrexate; cyclosporine; FK506; rapamycin; mycophenolate mofetil; leflunomide; NSAIDs, for example, ibuprofen; corticosteroids such as prednisolone; phosphodiesterase inhibitors; adenosine agonists; antithrombotic agents; complement inhibitors; adrenergic agents; agents which interfere with signaling by proinflammatory cytokines such as TNFα or IL-1 (e.g. NIK, IKK, or MAP kinase inhibitors); IL-1β converting enzyme inhibitors; TNFα converting enzyme inhibitors; T-cell signaling inhibitors such as kinase inhibitors; metalloproteinase inhibitors; sulfasalazine; azathioprine; 6-mercaptopurines; angiotensin converting enzyme inhibitors; soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-11, IL-13 and TGFβ). Preferred examples of therapeutic agents for Crohn's disease with which a compound of Formula (I) can be combined include the following: TNF antagonists, for example, anti-TNF antibodies, D2E7 (U.S. Pat. No. 6,090,382, HUMIRA™), CA2 (REMICADE™), CDP 571, TNFR-Ig constructs, (p75TNFRIgG (ENBREL™) and p55TNFRIgG (LENERCEPT™) inhibitors and PDE4 inhibitors. A compound of Formula (I) can be combined with corticosteroids, for example, budenoside and dexamethasone; sulfasalazine, 5-aminosalicylic acid; olsalazine; and agents which interfere with synthesis or action of proinflammatory cytokines such as IL-1, for example, IL-1β converting enzyme inhibitors and IL-1ra; T cell signaling inhibitors, for example, tyrosine kinase inhibitors; 6-mercaptopurine; IL-11; mesalamine; prednisone; azathioprine; mercaptopurine; infliximab; methylprednisolone sodium succinate; diphenoxylate/atrop sulfate; loperamide hydrochloride; methotrexate; omeprazole; folate; ciprofloxacin/dextrose-water; hydrocodone bitartrate/apap; tetracycline hydrochloride; fluocinonide; metronidazole; thimerosal/boric acid; cholestyramine/sucrose; ciprofloxacin hydrochloride; hyoscyamine sulfate; meperidine hydrochloride; midazolam hydrochloride; oxycodone HCl/acetaminophen; promethazine hydrochloride; sodium phosphate; sulfamethoxazole/trimethoprim; celecoxib; polycarbophil; propoxyphene napsylate; hydrocortisone; multivitamins; balsalazide disodium; codeine phosphate/apap; colesevelam HCl; cyanocobalamin; folic acid; levofloxacin; methylprednisolone; natalizumab and interferon-gamma.

Non-limiting examples of therapeutic agents for multiple sclerosis with which a compound of Formula (I) can be combined include the following: corticosteroids; prednisolone; methylprednisolone; azathioprine; cyclophosphamide; cyclosporine; methotrexate; 4-aminopyridine; tizanidine; interferon-β1a (AVONEX®; Biogen); interferon-β1b (BETASERON®; Chiron/Berlex); interferon α-n3) (Interferon Sciences/Fujimoto), interferon-α (Alfa Wassermann/J&J), interferon β1A-IF (Serono/Inhale Therapeutics), Peginterferon α 2b (Enzon/Schering-Plough), Copolymer 1 (Cop-1; COPAXONE®; Teva Pharmaceutical Industries, Inc.); hyperbaric oxygen; intravenous immunoglobulin; cladribine; antibodies to or antagonists of other human cytokines or growth factors and their receptors, for example, TNF, LT, IL-1, IL-2, IL-6, IL-7, IL-8, IL-12, IL-23, IL-15, IL-16, EMAP-II, GM-CSF, FGF, and PDGF. A compound of Formula (I) can be combined with antibodies to cell surface molecules such as CD2, CD3, CD4, CD8, CD19, CD20, CD25, CD28, CD30, CD40, CD45, CD69, CD80, CD86, CD90 or their ligands. A compound of Formula (I) may also be combined with agents such as methotrexate, cyclosporine, FK506, rapamycin, mycophenolate mofetil, leflunomide, an S1P1 agonist, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNFα or IL-1 (e.g., NIK, IKK, p38 or MAP kinase inhibitors), IL-1β converting enzyme inhibitors, TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-13 and TGFβ).

Preferred examples of therapeutic agents for multiple sclerosis in which a compound of Formula (I) can be combined to include interferon-β, for example, IFNβ1a and IFNβ1b; copaxone, corticosteroids, caspase inhibitors, for example inhibitors of caspase-1, IL-1 inhibitors, TNF inhibitors, and antibodies to CD40 ligand and CD80.

A compound of Formula (I) may also be combined with agents, such as alemtuzumab, dronabinol, daclizumab, mitoxantrone, xaliproden hydrochloride, fampridine, glatiramer acetate, natalizumab, sinnabidol, a-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist), MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists and IL-4 agonists.

Non-limiting examples of therapeutic agents for ankylosing spondylitis with which a compound of Formula (I) can be combined include the following: ibuprofen, diclofenac, misoprostol, naproxen, meloxicam, indomethacin, diclofenac, celecoxib, rofecoxib, sulfasalazine, methotrexate, azathioprine, minocyclin, prednisone, and anti-TNF antibodies, D2E7 (U.S. Pat. No. 6,090,382; HUMIRA™), CA2 (REMICADE™), CDP 571, TNFR-Ig constructs, (p75TNFRIgG (ENBREL™) and p55TNFRIgG (LENERCEPT™)

Non-limiting examples of therapeutic agents for psoriasis with which a compound of Formula (I) can be combined include the following: calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, sulfasalazine, ABT-874 and ustekinamab.

Non-limiting examples of therapeutic agents for psoriatic arthritis with which a compound of Formula (I) can be combined include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept, D2E7 (U.S. Pat. No. 6,090,382, HUMIRA™), and efalizumab.

Preferred examples of therapeutic agents for SLE (Lupus) with which a compound of Formula (I) can be combined include the following: NSAIDS, for example, diclofenac, naproxen, ibuprofen, piroxicam, indomethacin; COX2 inhibitors, for example, celecoxib, rofecoxib, valdecoxib; anti-malarials, for example, hydroxychloroquine; steroids, for example, prednisone, prednisolone, budenoside, dexamethasone; cytotoxics, for example, azathioprine, cyclophosphamide, mycophenolate mofetil, methotrexate; inhibitors of PDE4 or purine synthesis inhibitor, for example Cellcept®. A compound of Formula (I) may also be combined with agents such as sulfasalazine, 5-aminosalicylic acid, olsalazine, Imuran® and agents which interfere with synthesis, production or action of proinflammatory cytokines such as IL-1, for example, caspase inhibitors like IL-1β converting enzyme inhibitors and IL-1ra. A compound of Formula (I) may also be used with T cell signaling inhibitors, for example, tyrosine kinase inhibitors; or molecules that target T cell activation molecules, for example, CTLA-4-IgG or anti-B7 family antibodies, anti-PD-1 family antibodies. A compound of Formula (I) can be combined with IL-11 or anti-cytokine antibodies, for example, fonotolizumab (anti-IFNg antibody), or anti-receptor receptor antibodies, for example, anti-IL-6 receptor antibody and antibodies to B-cell surface molecules. A compound of Formula (I) may also be used with LJP 394 (abetimus), agents that deplete or inactivate B-cells, for example, Rituximab (anti-CD20 antibody), lymphostat-B (anti-BlyS antibody), TNF antagonists, for example, anti-TNF antibodies, D2E7 (U.S. Pat. No. 6,090,382; HUMIRA™), CA2 (REMICADE™), CDP 571, TNFR-Ig constructs, (p75TNFRIgG (ENBREL™) and p55TNFRIgG (LENERCEPT™).

In this invention, the following definitions are applicable:

A “therapeutically effective amount” is an amount of a compound of Formula (I) or a combination of two or more such compounds, which inhibits, totally or partially, the progression of the condition or alleviates, at least partially, one or more symptoms of the condition. A therapeutically effective amount can also be an amount which is prophylactically effective. The amount which is therapeutically effective will depend upon the patient's size and gender, the condition to be treated, the severity of the condition and the result sought. For a given patient, a therapeutically effective amount can be determined by methods known to those of skill in the art. “Pharmaceutically acceptable salts” refers to those salts which retain the biological effectiveness and properties of the free bases and which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, tartaric acid (e.g. (+) or (−)-tartaric acid or mixtures thereof), amino acids (e.g. (+) or (−)-amino acids or mixtures thereof), and the like. These salts can be prepared by methods known to those skilled in the art.

Certain compounds of Formula (I) which have acidic substituents may exist as salts with pharmaceutically acceptable bases. The present invention includes such salts. Examples of such salts include sodium salts, potassium salts, lysine salts and arginine salts. These salts may be prepared by methods known to those skilled in the art.

Certain compounds of Formula (I) and their salts may exist in more than one crystal form and the present invention includes each crystal form and mixtures thereof.
Certain compounds of Formula (I) and their salts may also exist in the form of solvates, for example hydrates, and the present invention includes each solvate and mixtures thereof.

Certain compounds of Formula (I) may contain one or more chiral centers, and exist in different optically active forms. When compounds of Formula (I) contain one chiral center, the compounds exist in two enantiomeric forms and the present invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of Formula (I) contains more than one chiral center, it may exist in diastereoisomeric forms. The diastereoisomeric compounds may be separated by methods known to those skilled in the art, for example chromatography or crystallization and the individual enantiomers may be separated as described above. The present invention includes each diastereoisomer of compounds of Formula (I), and mixtures thereof. Certain compounds of Formula (I) may exist in different tautomeric forms or as different geometric isomers, and the present invention includes each tautomer and/or geometric isomer of compounds of Formula (I) and mixtures thereof. Certain compounds of Formula (I) may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of Formula (I) and mixtures thereof. Certain compounds of Formula (I) may exist in zwitterionic form and the present invention includes each zwitterionic form of compounds of Formula (I) and mixtures thereof.

As used herein the term “pro-drug” refers to an agent which is converted into the parent drug in vivo by some physiological chemical process (e.g., a pro-drug on being brought to the physiological pH is converted to the desired drug form). Pro-drugs are often useful because, in some situations, they may be easier to administer than the parent drug. They may, for instance, be bioavailable by oral administration whereas the parent drug is not. The pro-drug may also have improved solubility in pharmacological compositions over the parent drug. An example, without limitation, of a pro-drug would be a compound of the present invention wherein it is administered as an ester (the “pro-drug”) to facilitate transmittal across a cell membrane where water solubility is not beneficial, but then it is metabolically hydrolyzed to the carboxylic acid once inside the cell where water solubility is beneficial.

Pro-drugs have many useful properties. For example, a pro-drug may be more water soluble than the ultimate drug, thereby facilitating intravenous administration of the drug. A pro-drug may also have a higher level of oral bioavailability than the ultimate drug. After administration, the pro-drug is enzymatically or chemically cleaved to deliver the ultimate drug in the blood or tissue.

Exemplary pro-drugs upon cleavage release the corresponding free acid, and such hydrolyzable ester-forming residues of the compounds of this invention include but are not limited to phosphates, phosphate esters, and carboxylic acid substituents wherein the free hydrogen is replaced by (C₁-C₄)alkyl, (C₁-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino (C₂-C₃)alkyl (such as β-dimethylaminoethyl), carbamoyl-(C₁-C₂)alkyl, N,N-di(C₁-C₂)-alkylcarbamoyl-(C₁-C₂)alkyl and piperidino-, pyrrolidino- or morpholino(C₂-C₃)alkyl.

Other exemplary pro-drugs release an alcohol of Formula (I) wherein the free hydrogen of the hydroxyl substituent is replaced by (C₁-C₆)alkanoyloxymethyl, 1((C₁-C₆)alkanoyloxy)ethyl, 1-methyl-1-((C₁-C₆)alkanoyloxy)ethyl, (C₁-C₁₂)alkoxycarbonyloxymethyl, N—(C₁-C₆)alkoxycarbonylamino-methyl, succinoyl, (C₁-C₆)alkanoyl, α-amino(C₁-C₄)alkanoyl, arylacetyl and α-aminoacyl, or α-aminoacyl-α-aminoacyl wherein said α-aminoacyl moieties are independently any of the naturally occurring L-amino acids found in proteins, P(O)(OH)₂, —P(O)(O(C₁-C₆)alkyl)₂ or glycosyl (the radical resulting from detachment of the hydroxyl of the hemiacetal of a carbohydrate).

Other exemplary pro-drugs release an amine of Formula (I) wherein the free hydrogen of the amine group is replaced by —C(O)alkyl, —C(O)O-alkyl, N-phosphonoxyalkyl, alkyl, cycloalkyl, aryl, heteroaryl or heterocyclyl, wherein the alkyl, cycloalkyl, aryl, heteroaryl or heterocyclyl can be optionally substituted with, for example, halogen and hydroxyl.

As used herein “solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like.

As used herein, “spirocyclic (C₂-C₁₀) heterocyclyl” means bicyclic or polycyclic hydrocarbon group having two or three (C₃-C₁₀) rings at least one of which contains a heteroatom such as nitrogen, oxygen or sulfur. For purposes of exemplification, which should not be construed as limiting the scope of this invention, spirocyclic (C₂-C₁₀) heterocyclyl may include diazaspiro[3.5]nonane and diazaspiro[4.5]decane.

As used herein, “spirocyclic (C₅-C₁₁) carbocyclyl” means a saturated or unsaturated, bicyclic or polycyclic hydrocarbon group having two or three (C₃-C₁₀) cycloalkyl rings. For purposes of exemplification, which should not be construed as limiting the scope of this invention, spirocyclic (C₅-C₁₁) carbocyclyl includes spiro[5.5]undecane, spiro[4.5]decane and spiro[4.4]nonane.

The term “heterocyclic”, “heterocyclyl” or “heterocyclylene”, as used herein, include non-aromatic ring systems, including, but not limited to, monocyclic, bicyclic, and tricyclic rings, which can be completely saturated or which can contain one or more units of unsaturation. (for the avoidance of doubt, the degree of unsaturation does not result in an aromatic ring system) and have 5 to 12 atoms including at least one heteroatom, such as nitrogen, oxygen, or sulfur. For purposes of exemplification, which should not be construed as limiting the scope of this invention, the following are examples of heterocyclic rings: azepinyl, azetidinyl, indolinyl, isoindolinyl, morpholinyl, piperazinyl, piperidinyl, pyrrolidinyl, quinucludinyl, thiomorpholinyl, tetrahydropyranyl, tetrahydrofuranyl, tetrahydroindolyl, thiomorpholinyl and tropanyl.

The term “heteroaryl” or “heteroarylene” as used herein, include aromatic ring systems, including, but not limited to, monocyclic, bicyclic and tricyclic rings, and have 5 to 12 atoms including at least one heteroatom, such as nitrogen, oxygen, or sulfur. For purposes of exemplification, which should not be construed as limiting the scope of this invention: azaindolyl, benzo[b]thienyl, benzimidazolyl, benzofuranyl, benzoxazolyl, benzothiazolyl, benzothiadiazolyl, benzoxadiazolyl, 6,7-dihydro-5H-cyclopentapyrimidinyl, furanyl, imidazolyl, imidazopyridinyl, indolyl, indazolyl, isoxazolyl, isothiazolyl, octahydro-pyrrolopyrrolyl, oxadiazolyl, oxazolyl, phthalazinyl, pteridinyl, purinyl, pyranyl, 5,8-dihydro-6H-pyrano[3,4-d]pyridinyl, pyrazinyl, pyrazolyl, pyridinyl, pyrido[2,3-d]pyrimidinyl, pyrido[4,3-d]pyrimidinyl, pyrido[3,4-d]pyrimidinyl, pyrimidinyl, pyrimido[4,5-d]pyrimidinyl, pyrrolyl, pyrrolo[2,3-d]pyrimidinyl, pyrazolo[3,4-d]pyrimidinyl, quinolinyl, quinazolinyl, 5,6,7,8-tetrahydroquinazolinyl, triazolyl, thiazolyl, thieno[2,3-d]pyrimidinyl, thieno[3,2-d]pyrimidinyl, thiophenyl, tetrazolyl, thiadiazolyl, thienyl, [1,3,5]triazinyl, 5,6,7,8-tetrahydro-imidazo[1,5-a]pyrazinyl, and 5,6,7,8-tetrahydro-triazolo[1,2,4]pyrazinyl.

As used herein, “alkyl” and “alkylene” include straight chained or branched hydrocarbons which are completely saturated. For purposes of exemplification, which should not be construed as limiting the scope of this invention, examples of alkyls are methyl, ethyl, propyl, isopropyl, butyl, pentyl, hexyl and isomers thereof.

As used herein, “alkenyl”, “alkenylene”, “alkynylene” and “alkynyl” mean hydrocarbon moieties containing two to eight carbons and include straight chained or branched hydrocarbons which contain one or more units of unsaturation, one or more double bonds for alkenyl and one or more triple bonds for alkynyl. For purposes of exemplification, which should not be construed as limiting the scope of this invention, examples of alkenyl are ethenyl, propenyl and butenyl, and examples of alkynyl are ethynyl, propynyl and butynyl.

As used herein, “aryl” or “arylene” groups include aromatic carbocyclic ring systems (e.g. phenyl) and fused polycyclic aromatic ring systems. For purposes of exemplification, which should not be construed as limiting the scope of this invention, aryl groups include naphthyl, biphenyl and 1,2,3,4-tetrahydronaphthyl.

As used herein, “cycloalkyl”, “cycloalkylene”, “carbocycle” or “carbocyclyl” means C₃-C₁₂ monocyclic or multicyclic (e.g., bicyclic, tricyclic, etc.) hydrocarbons that are completely saturated or have one or more unsaturated bonds but do not amount to an aromatic group. For purposes of exemplification, which should not be construed as limiting the scope of this invention, examples of a cycloalkyl group are cyclopropyl, cyclobutyl, cyclopentyl, cyclopentenyl, cyclohexyl and cyclohexenyl.

As used herein, many moieties or substituents are termed as being either “substituted” or “optionally substituted”. When a moiety is modified by one of these terms, unless otherwise noted, it denotes that any portion of the moiety that is known to one skilled in the art as being available for substitution can be substituted, which includes one or more substituents, where if more than one substituent then each substituent is independently selected. Such means for substitution are well-known in the art and/or taught by the instant disclosure. For purposes of exemplification, which should not be construed as limiting the scope of this invention, some examples of groups that are substituents are: deuterium, CD₃, optionally substituted (C₁-C₈)alkyl groups, optionally substituted (C₂-C₈)alkenyl groups, (C₂-C₈)alkynyl groups, optionally substituted (C₃-C₁₀)cycloalkyl groups, halogen (F, Cl, Br or I), halogenated (C₁-C₈)alkyl groups (for example but not limited to —CF₃), —O—(C₁-C₈)alkyl groups, —OH, —S—(C₁-C₈)alkyl groups, —SH, —NH(C₁-C₈)alkyl groups, —N((C₁-C₈)alkyl)₂ groups, —NH₂, —NH—(C₁-C₆)alkyl-optionally substituted heterocycle, —NH-heterocycle, —C(O)NH₂, —C(O)NH(C₁-C₈)alkyl groups, —C(O)N((C₁-C₈)alkyl)₂, —NHC(O)H, —NHC(O)(C₁-C₈)alkyl groups, —NHC(O)(C₃-C₈)cycloalkyl groups, —N((C₁-C₈)alkyl)C(O)H, —N((C₁-C₈)alkyl)C(O)(C₁-C₈)alkyl groups, —NHC(O)NH₂, —NHC(O)NH(C₁-C₈)alkyl groups, —N((C₁-C₈)alkyl)C(O)NH₂ groups, —NHC(O)N((C₁-C₈)alkyl)₂ groups, —N((C₁-C₈)alkyl)C(O)N((C₁-C₈)alkyl)₂ groups, —N((C₁-C₈)alkyl)C(O)NH((C₁-C₈)alkyl), —C(O)H, —C(O)(C₁-C₈)alkyl groups, —CN, —NO₂, —S(O)(C₁-C₈)alkyl groups, —S(O)₂(C₁-C₈)alkyl groups, —S(O)₂N((C₁-C₈)alkyl)₂ groups, —S(O)₂NH(C₁-C₈)alkyl groups, —S(O)₂NH(C₃-C₈)cycloalkyl groups, —S(O)₂NH₂ groups, —NHS(O)₂(C₁-C₈)alkyl groups, —N((C₁-C₈)alkyl)S(O)₂(C₁-C₈)alkyl groups, —(C₁-C₈)alkyl-O—(C₁-C₈)alkyl groups, —O—(C₁-C₈)alkyl-O—(C₁-C₈)alkyl groups, —C(O)OH, —C(O)O(C₁-C₈)alkyl groups, —NHOH, —NHO(C₁-C₈)alkyl groups, —O-halogenated (C₁-C₈)alkyl groups (for example but not limited to —OCF₃), —S(O)₂-halogenated (C₁-C₈)alkyl groups (for example but not limited to —S(O)₂CF₃), —S-halogenated (C₁-C₈)alkyl groups (for example but not limited to —SCF₃), —(C₁-C₆)alkyl-optionally substituted heterocycle (for example but not limited to azetidine, piperidine, piperazine, pyrrolidine, tetrahydrofuran, pyran or morpholine), —(C₁-C₆)alkyl-heteroaryl (for example but not limited to tetrazole, imidazole, furan, pyrazine or pyrazole), -optionally substituted phenyl, —NHC(O)O—(C₁-C₆)alkyl groups, —N((C₁-C₆)alkyl)C(O)O—(C₁-C₆)alkyl groups, —C(═NH)—(C₁-C₆)alkyl groups, —C(═NOH)—(C₁-C₆)alkyl groups, or —C(═N—O—(C₁-C₆)alkyl)-(C₁-C₆)alkyl groups.

One or more compounds of this invention can be administered to a human patient by themselves or in pharmaceutical compositions where they are mixed with biologically suitable carriers or excipient(s) at doses to treat or ameliorate a disease or condition as described herein. Mixtures of these compounds can also be administered to the patient as a simple mixture or in suitable formulated pharmaceutical compositions. A therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

Suitable routes of administration may, for example, include oral, eyedrop, rectal, transmucosal, topical, inhaled or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds can be formulated for parenteral administration by injection, e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few hours up to over several days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art.

For any compound used in a method of the present invention, the therapeutically effective dose can be estimated initially from cellular assays. For example, a dose can be formulated in cellular and animal models to achieve a circulating concentration range that includes the IC₅₀ as determined in cellular assays (i.e., the concentration of the test compound which achieves a half-maximal inhibition of a given protein kinase activity). In some cases it is appropriate to determine the IC₅₀ in the presence of 3 to 5% serum albumin since such a determination approximates the binding effects of plasma protein on the compound. Such information can be used to more accurately determine useful doses in humans. Further, the most preferred compounds for systemic administration effectively inhibit protein kinase signaling in intact cells at levels that are safely achievable in plasma.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the maximum tolerated dose (MTD) and the ED₅₀ (effective dose for 50% maximal response). The dose ratio between toxic and therapeutic effects is the therapeutic index and it can be expressed as the ratio between MTD and ED₅₀. Compounds which exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition (see e.g. Fingl et al., 1975, in “The Pharmacological Basis of Therapeutics”, Ch. 1 p. 1). In the treatment of crises, the administration of an acute bolus or an infusion approaching the MTD may be required to obtain a rapid response.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g. the concentration necessary to achieve 50-90% inhibition of protein kinase using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and most preferably between 50-90% until the desired amelioration of symptoms is achieved. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labelled for treatment of an indicated condition.

In some formulations it may be beneficial to use the compounds of the present invention in the form of particles of very small size, for example as obtained by fluid energy milling.

The use of compounds of the present invention in the manufacture of pharmaceutical compositions is illustrated by the following description. In this description the term “active compound” denotes any compound of the invention but particularly any compound which is the final product of one of the following Examples.

### a) Capsules

In the preparation of capsules, 10 parts by weight of active compound and 240 parts by weight of lactose can be de-aggregated and blended. The mixture can be filled into hard gelatin capsules, each capsule containing a unit dose or part of a unit dose of active compound.

### b) Tablets

Tablets can be prepared, for example, from the following ingredients.

Parts by weight

| | |
|---|---|
| Active compound | 10 |
| Lactose | 190 |
| Maize starch | 22 |
| Polyvinylpyrrolidone | 10 |
| Magnesium stearate | 3 |

The active compound, the lactose and some of the starch can be de-aggregated, blended and the resulting mixture can be granulated with a solution of the polyvinylpyrrolidone in ethanol. The dry granulate can be blended with the magnesium stearate and the rest of the starch. The mixture is then compressed in a tabletting machine to give tablets each containing a unit dose or a part of a unit dose of active compound.

### c) Enteric Coated Tablets

Tablets can be prepared by the method described in (b) above. The tablets can be enteric coated in a conventional manner using a solution of 20% cellulose acetate phthalate and 3% diethyl phthalate in ethanol:dichloromethane (1:1).

### d) Suppositories

In the preparation of suppositories, for example, 100 parts by weight of active compound can be incorporated in 1300 parts by weight of triglyceride suppository base and the mixture formed into suppositories each containing a therapeutically effective amount of active ingredient.

In the compositions of the present invention the active compound may, if desired, be associated with other compatible pharmacologically active ingredients. For example, the compounds of this invention can be administered in combination with another therapeutic agent that is known to treat a disease or condition described herein. For example, with one or more additional pharmaceutical agents that inhibit or prevent the production of VEGF or angiopoietins, attenuate intracellular responses to VEGF or angiopoietins, block intracellular signal transduction, inhibit vascular hyperpermeability, reduce inflammation, or inhibit or prevent the formation of edema or neovascularization. The compounds of the invention can be administered prior to, subsequent to or simultaneously with the additional pharmaceutical agent, whichever course of administration is appropriate. The additional pharmaceutical agents include, but are not limited to, anti-edemic steroids, NSAIDS, ras inhibitors, anti-TNF agents, anti-IL1 agents, antihistamines, PAF-antagonists, COX-1 inhibitors, COX-2 inhibitors, NO synthase inhibitors, Akt/PTB inhibitors, IGF-1R inhibitors, PI3 kinase inhibitors, calcineurin inhibitors and immunosuppressants. The compounds of the invention and the additional pharmaceutical agents act either additively or synergistically. Thus, the administration of such a combination of substances that inhibit angiogenesis, vascular hyperpermeability and/or inhibit the formation of edema can provide greater relief from the deletrious effects of a hyperproliferative disorder, angiogenesis, vascular hyperpermeability or edema than the administration of either substance alone. In the treatment of malignant disorders combinations with antiproliferative or cytotoxic chemotherapies or radiation are included in the scope of the present invention.

The present invention also comprises the use of a compound of Formula (I) as a medicament.

### Purification Methods

Intermediate and final compounds may be purified by any technique or combination of techniques known to one skilled in the art. Some examples that are not limiting include flash chromatography with a solid phase (i.e. silica gel, alumina, etc.) and a solvent (or combination of solvents, i.e. heptane, EtOAc, DCM, MeOH, MeCN, water, etc.) that elutes the desired compounds; preparatory TLC with a solid phase (i.e. silica gel, alumina etc.) and a solvent (or combination of solvents, i.e. heptane, EtOAc, DCM, MeOH, MeCN, water, etc.) that elutes the desired compounds; reverse phase HPLC (see Table 1 for some non-limiting conditions); recrystallization from an appropriate solvent (i.e. MeOH, EtOH, i-PrOH, EtOAc, toluene, etc.) or combination of solvents (i.e. EtOAc/heptane, EtOAc/MeOH, etc.); chiral chromatography with a solid phase and an appropriate solvent (i.e. EtOH/heptane, MeOH/heptane, i-PrOH/heptane, etc. with or without a modifier such as diethylamine, TFA, etc.) to elute the desired compound; precipitation from a combination of solvents (i.e. DMF/water, DMSO/DCM, EtOAc/heptane, etc.); trituration with an appropriate solvent (i.e. EtOAc, DCM, MeCN, MeOH, EtOH, i-PrOH, n-PrOH, etc.); extractions by dissolving a compound in a liquid and washing with an appropriately immiscible liquid (i.e. DCM/water, EtOAc/water, DCM/saturated NaHCO₃, EtOAc/saturated NaHCO₃, DCM/10% aqueous HCl, EtOAc/10% aqueous HCl, etc.); distillation (i.e. simple, fractional, Kugelrohr, etc.); gas chromatography using an appropriate temperature, carrier gas and flow rate; sublimation at an appropriate temperature and pressure; filtration through a media (i.e. Florosil®, alumina, Celite®, silica gel, etc.) with a solvent (i.e. heptane, hexanes, EtOAc, DCM, MeOH, etc.) or combination of solvents; salt formation with solid support (resin based, i.e. ion exchange) or without. Descriptions of these techniques can be found in the following references: Gordon, A. J. and Ford, R. A. “The Chemist's Companion”, 1972; Palleros, D. R. “Experimental Organic Chemistry”, 2000; Still, W. C., Kahn and M. Mitra, A. J. Org. Chem. 1978, 43, 2923; Yan, B. “Analysis and Purification Methods in Combinatorial Chemistry” 2003; Harwood, L. M., Moody, C. J. and Percy, J. M. “Experimental Organic Chemistry: Standard and Microscale, 2^{nd} Edition”, 1999; Stichlmair, J. G. and Fair, J. R. “Distillation; Principles and Practices” 1998; Beesley T. E. and Scott, R. P. W. “Chiral Chromatography”, 1999; Landgrebe, J. A. “Theory and Practice in the Organic Laboratory, 4^{th} Ed.”, 1993; Skoog, D. A. and Leary, J. J. “Principles of Instrumental Analysis, 4^{th} Ed.” 1992; G. Subramanian, “Chiral Separation Techniques 3^{rd} Edition” 2007; Y. Kazakevich, R. Lobrutto, “HPLC for Pharmaceutical Scientists” 2007.

### Degassing Methods

Preparations of intermediate and final compounds obtained via the General Procedures can be optionally degassed using one or more of the Degassing Methods described below. The reaction mixtures may be degassed by a single or multiple applications of any technique or combination of techniques known to one skilled in the art. Some examples that are not limiting include bubbling a continuous stream of an inert gas (e.g. nitrogen, argon, etc.) through a mixture of reagents and a solvent suitable for the transformation (e.g. THF, 1,4-dioxane, EtOAc, DCM, toluene, MeOH, EtOH, DMF, MeCN, water, etc.); freeze-thawing of a mixture of reagents in a solvent (e.g. THF, 1,4-dioxane, EtOAc, DCM, toluene, MeOH, EtOH, DMF, MeCN, water, etc.) where the resulting solution is cooled below its freezing point and evacuated under reduced pressure, then allowed to warm above the freezing point and purged with an atmosphere of inert gas (e.g. nitrogen, argon, etc.); evacuation under reduced pressure of a mixture of reagents with or without a suitable solvent for the transformation (e.g. THF, 1,4-dioxane, EtOAc, DCM, toluene, MeOH, EtOH, DMF, MeCN, water, etc.) followed by purging of the mixture with an inert gas (e.g. nitrogen, argon, etc.); evacuation under reduced pressure of a mixture of reagents in a suitable solvent for the transformation (e.g. THF, 1,4-dioxane, EtOAc, DCM, toluene, MeOH, EtOH, DMF, MeCN, water, etc.) with the aid of mechanical agitation (e.g. stirring, shaking, sonication, etc.) followed by purging of the mixture with an inert gas (e.g. nitrogen, argon, etc.). Some descriptions of these techniques can be found in the following references, Gordon, A. J. and Ford, R. A. “The Chemist's Companion”, 1972; Palleros, D. R. “Experimental Organic Chemistry”, 2000; Harwood, L. M., Moody, C. J. and Percy, J. M. “Experimental Organic Chemistry: Standard and Microscale, 2^{nd} Edition”, 1999; Landgrebe, J. A. “Theory and Practice in the Organic Laboratory, 4^{th} Edition”, 1993; Leonard, J., Lygo, B. and Procter, G. “Advanced Practical Organic Chemistry, 2^{nd} Edition”, 1998; Meyers, A. G.; Dragovich, P. S. Organic Syntheses, 1995, 72, 104; Hajos, Z. G., Parrish, D. R. Organic Syntheses, 1985, 63, 26.

### EXAMPLES

None of the specific conditions and reagents noted herein are to be construed as limiting the scope of the invention and are provided for illustrative purposes only. All starting materials are commercially available from Sigma-Aldrich (including Fluka and Discovery CPR) unless otherwise noted after the chemical name. Reagent/reactant names given are as named on the commercial bottle or as generated by IUPAC conventions, CambridgeSoft® Chemdraw Ultra 9.0.7 or AutoNom 2000. Compounds designated as salts (e.g. hydrochloride, acetate) may contain more than one molar equivalent of the salt.

### ABBREVIATIONS

Ac Acetyl
AcOH Glacial acetic acid
Bs Broad singlet
BTFFH Fluoro-N,N,N′,N′-bis(tetramethylene)formamidinium hexafluorophosphate
COMU (1-Cyano-2-ethoxy-2-oxoethylidenaminooxy)dimethylamino-morpholino-carbenium hexafluorophosphate
d Doublet
DAD Diode array detection
dba Dibenzylideneacetone
DBAD Di-tert-butyl azodicarboxylate
DCE 1,2-Dichloroethane
DCM Dichloromethane (methylene chloride)
dd Doublet of doublets
DEA Diethylamine
Dess-Martin periodinane 1,1,1-Tris(acetyloxy)-1,1-dihydro-1,2-benziodoxol-3-(1H)-one
DIEA Diisopropylethylamine
DME 1,2-Dimethoxyethane
DMEM/F12 Dulbecco's Modified Eagle's Medium: Nutrient Mixture F-12
DMF N,N-Dimethylformamide
DMS Dimethylsulfide
DMSO Dimethyl sulfoxide
dppf (diphenylphosphino)ferrocene
EDTA Ethylenediaminetetraacetic acid
ELSD Evaporative light scattering detection
EtOAc Ethyl acetate
Et₂O Diethyl ether
EtOH Ethanol
FBS Fetal bovine serum
g Gram(s)
GR Glucocorticiod receptor
h Hour(s)
HBTU 2-(1H-benzo[d][1,2,3]triazol-1-yl)-1,1,3,3-tetramethylis ouronium hexafluorophosphate(V)
HEPES N-2-Hydroxyethylpiperazine-N′-2-ethanesulfonic acid
Hz Hertz
L Liter(s)
LC Liquid chromatography
LDA Lithium diisopropylamide
LiHMDS Lithium Hexamethyldisilazide
LiOH Lithium hydroxide
m Multiplet
M Molar
MeCN Acetonitrile
MeOH Methyl alcohol
min Minute(s)
mL Milliliter(s)
mmol Millimole(s)
mM Millimolar
mm Millimeter(s)
MS Mass spectrometry
MTBE Methyl tert-butyl ether
N Normal
ng Nanogram(s)
NH₄OAc Ammonium acetate
nM Nanomolar
NMO 4-Methylmorphloine N-oxide
NMR Nuclear magnetic resonance
OCN Osteocalcin
Pd₂(dba)₃ Tris(dibenzylideneacetone)dipalladium(0)
PPh₃ Triphenylphosphine
psi Pounds per square inch
PS-PPh₃ Polymer-supported triphenylphosphine
R_{f} Retention factor
rpm Revolutions per minute
Rₜ Retention time
rt Room temperature
s Singlet
SFC Supercritical fluid chromatography
t Triplet
TBDMS tert-Butyldimethylsilyl
TBDMSCI tert-Butyldimethylsilyl chloride
TBAB Tetra-n-butylammonium bromide
TBAF Tetra-n-butylammonium fluoride
TBAI Tetra-n-butylammonium iodide
Tfa Trifluoroacetic acid
TEA Triethylamine
TES Triethylsilyl
Tf Trifluoromethanesulfonyl
TFFH Fluoro-N,N,N′,N′-tetramethylformamidinium hexafluorophosphate
THF Tetrahydrofuran
TPAP Tetrapropylammonium perruthenate
TPP 2,4,6-Tripropyl-[1,3,5,2,4,6]trioxatriphosphinane 2,4,6-trioxide
U Unit(s)
Wt Weight
Xantphos 4,5-Bis(diphenylphosphino)-9,9-dimethylxanthene
μL Microliter(s)
μg Microgram(s)
μM Micromolar
μm Micrometer(s)

### Methods:

### GR Florescence Polarization Assay

Florescence polarization assays were carried out using the PolarScreen™ Glucocorticoid Receptor Competitor Assay, Red from Invitrogen (P2893). The assay buffer was prepared according to the manufacturer's protocol and used to dilute the fluorescent glucocorticoid and GR. Compounds were prepared and serial diluted 1:4 in DMSO. Compound, fluorescent glucocorticoid and GR were added in a final volume of 20 μL and incubated overnight at 4° C. Fluorescent polarization was measured on the Perkinelmer Envision®.

### A549 Cell Assay to Measure Inflammation Markers

A549 cells were seeded (3E4 cells/well) in 96-well assay plates in culture medium (100 μL/well., F-12 K base media, supplemented with 10% FBS and 100 μ/mL-100 μg/mL Pen-Strep.) After overnight culture in an incubator set to 37° C., 4.9% CO₂, and 90% humidity, media was removed from adherent cells by aspiration and replaced with 100 μL/well Assay Medium (F-12 K base media supplemented with 5% charcoal stripped calf sera and 100 U/mL-100 μg/mL Pen-Strep.) Compounds were prepared in DMSO and serial diluted (1:3, 1:4, or 1:5) with DMSO in Dilution Plate(s) to give 10 dilution points for each compound tested. Further dilution (1:250) of compound was made into assay medium and 50 μL/well diluted drug or DMSO/media control was applied to cells. After a 1 h pre-incubation in a temperature, CO₂, and humidity controlled incubator, set to 37° C., 50 μL/well of 4 ng/mL IL-1β diluted in assay media, was applied to cultures. Assay plates, with a final volume of 200 μL/well and final concentrations of 0.1% DMSO and 1 ng/mL IL-1β were returned to incubator for a four h incubation period. Next, plates were spun at 183 g (1000 rpm in Beckman/Coulter Allegra 6KR centrifuge) for 10 min. Cell-free supernatant (150 μL/well) was collected and IL-6 was measured by MSD kit, following protocol of manufacturer, and using MSD SECTOR Imager 6000 instrument. Potency of compounds to inhibit IL-6 was determined using the percent reduction of measured IL-6 in wells with compound compared to control wells without drug, and relative to (100% inhibition) positive control compound of 10 μM prednisolone. Results were represented as IC₅₀ and Emax values. To verify that viable cell numbers were similar across plate(s), and not confounding compound IC₅₀ data interpretation, the remaining 50 μL/well of cells and media (after removal of supernatant) were used to run Cell Titer-Glo Assay per directions of manufacturer.

MG-63 Cell Assay to Measure Bone Markers MG-63 cells were cultured in culture media containing ascorbic acid (DMEM/F12 supplemented with 10% FBS, 1% HEPES, 100 U/mL-100 μg/mL Pen-Strep, and 100 μg/mL of ascorbic acid) for, minimally, 1 week before study. MG-63 cells were seeded (4E4 cells/well) in 96-well assay plates in culture medium (200 μL/well.) After overnight culture in an incubator set to 37° C., 4.9% CO₂, and 90% humidity, media was removed from adherent cells by aspiration and replaced with 100 μL/well assay medium, DMEM/F12 supplemented with 5% Charcoal Stripped Serum, 1% HEPES, 100 U/mL-100 μg/mL Pen-Strep, and 100 μg/mL of ascorbic acid. Compounds were prepared with DMSO and serial diluted (1:3, 1:4, or 1:5) with DMSO in dilution plate(s) to give 10 dilution points for each compound tested. Further dilution (1:250) of compound was made into assay medium and 50 μL/well diluted drug or DMSO/media control was applied to cells. After a 1 h pre-incubation in a temperature, CO₂, and humidity controlled incubator, set to 37° C., 50 μL/well of 40 nM Vitamin K and 400 nM Vitamin D that were diluted in assay media were applied to plates. Assay plates, with a final volume of 200 μL/well and final concentrations of 0.1% DMSO, 10 nM Vitamin K, and 100 nM Vitamin D, were returned to incubator for overnight culture. Next, plates were spun at 183 g (1000 rpm in Beckman/Coulter Allegra 6KR centrifuge) for 10 min. Cell-free supernatant (150 μL/well) was collected and OCN was measured by MSD kit, following protocol of manufacturer, and using MSD SECTOR Imager 6000 instrument. Potency of drug to inhibit OCN was determined using the percent reduction of measured OCN in wells with drug compared to control wells without drug, and relative to (100% inhibition) positive control sample of 10 μM prednisolone. Results were represented as IC₅₀ and Emax values. To verify that viable cell numbers were similar across plate(s), and not confounding compound IC₅₀ data interpretation, the remaining 50 μL/well of cells and media (after removal of supernatant) were used to run Cell Titer-Glo Assay per directions of manufacturer.

### LC/MS Methods

Method 1: HPLC 2 min method: The gradient was 5-60% B in 0.60 min then 60-95% B to 1.0 min with a hold at 95% B for 0.30 (1.25 mL/min flow rate). The column used for the chromatography is 2.1×30 mm Acquity HPLC HSS T3 column (1.8 mm particles). The gradient was 5-60% B in 0.60 min then 60-95% B to 1.0 min with a hold at 95% B for 0.30 (1.25 mL/min flow rate). The mobile phase A was 10 mM NH₄OAc, mobile phase B was HPLC grade MeCN. Detection methods are diode array (DAD) and evaporative light scattering (ELSD) detection as well as pos/neg electrospray ionization

Method 2: Halo Purity QC method: The gradient was 5-60% B in 1.5 min then 60-95% B to 2.5 min with a hold at 95% B for 1.2 min (1.3 mL/min flow rate). The mobile phase A was 10 mM NH₄OAc, mobile phase B was HPLC grade MeCN. The column used for the chromatography is a 4.6×50 mm MAC-MOD Halo C18 column (2.7 μm particles). Detection methods are diode array (DAD) and evaporative light scattering (ELSD) detection as well as positive/negative electrospray ionization.

Method 3: Halo 4 min method: The gradient was 5-60% B in 1.5 min then 60-95% B to 2.5 min with a hold at 95% B for 1.2 min (1.3 mL/min flow rate). The mobile phase A was 10 mM NH₄OAc, mobile phase B was HPLC grade MeCN. The column used for the chromatography is a 4.6×50 mm MAC-MOD Halo C8 column (2.7 μm particles). Detection methods are diode array (DAD) and evaporative light scattering (ELSD) detection as well as positive/negative electrospray ionization.

Method 4: Halo test 4 min nonpolar; (30-95%: 4 min gradient for highly nonpolar): The gradient was 30-60% B in 1.50 min then 60-95% B to 2.5 min with a hold at 95% B for 1.2 min (1.3 mL/min flow rate). The mobile phase A was 10 mM ammonium acetate, mobile phase B was HPLC grade MeCN. The column used for the chromatography is a 4.6×50 mm MAC-MOD Halo C8 column (2.7 μm particles). Detection methods are diode array (DAD) and evaporative light scattering (ELSD) detection as well as positive/negative electrospray ionization.

### Analytical Chiral Chromatography Methods

### Method A:

(SFC) Gradient separation method wherein mobile phase A was SFC grade CO₂; mobile phase B was HPLC grade MeOH with 0.1% DEA. The gradient was 10% co-solvent B for 1 min then 10-55% mobile phase B in 6 min with a hold at 55% for 1 min (4 mL/min, 100 bar system pressure). The column used for the chromatography was a 4.6×250 mm Diacel IB column. Detection methods are diode array (DAD) and positive/negative electrospray ionization.

### Method B:

(SFC) Gradient separation method wherein mobile phase A was SFC grade CO₂; mobile phase B was HPLC grade isopropyl alcohol with 0.1% DEA. The gradient was 10% co-solvent B for 1 min then 10-55% mobile phase B in 6 min with a hold at 55% for 1 min (4 mL/min, 100 bar system pressure). The column used for the chromatography was a 4.6×250 mm Diacel IA (5 μm particles). Detection methods are diode array (DAD) and positive/negative electrospray ionization.

### Method C:

(SFC) Gradient separation method wherein mobile phase A was SFC grade CO₂; mobile phase B was HPLC grade EtOH with 0.1% DEA. The gradient was 10% co-solvent B for 1 min then 10-55% mobile phase B in 6 min with a hold at 55% for 1 min (4 mL/min, 100 bar system pressure). The column used for the chromatography was a 4.6×250 mm Diacel IA (5 μm particles). Detection methods are diode array (DAD) and positive/negative electrospray ionization.

### Method D:

(SFC) Gradient, 10% co-solvent B for 1 min then 10 to 55% B over 6 min then hold at 55% B for 1 min then 55% to 10% B over 1 min, total run time 9 min (Total flow 4 mL/min, 100 bar system pressure, 40° C.). Co-solvent B was MeOH with 0.1% DEA added. Solvent A was SFC grade CO₂. The column used for the chromatography was a 4.6×250 mm Daicel IA column from (5 μm particles). Detection methods are diode array (DAD) and evaporative light scattering (ELSD) detection as well as pos/neg electrospray ionization

### Preparative Chiral Chromatography Methods

### Method 1:

(SFC) Isocratic, 30% co-solvent B (80 mL/min, 100 bar system pressure, 25° C.). Co-solvent B was 1:1 HPLC grade MeOH:isopropanol. Solvent A was SFC grade CO₂. The column used for the chromatography was a 30×250 mm ChiralPak AD-H from Chiral Technologies (5 μm particles).

### Method 2:

(SFC) Isocratic, 27% co-solvent B (80 mL/min, 100 bar system pressure, 25° C.). Co-solvent B was 1:1 HPLC grade MeOH:isopropanol. Solvent A was SFC grade CO₂. The column used for the chromatography was a 30×250 mm RegisPack from Regis Technologies (5 μm particles).

### Method 3:

(SFC) Isocratic, 25% co-solvent B (80 mL/min, 100 bar system pressure, 25° C.). Co-solvent B was 1:1 HPLC grade MeOH:isopropanol. Solvent A was SFC grade CO₂. The column used for the chromatography was a 30×250 mm ChiralPak AD-H from Chiral Technologies (5 μm particles).

### Method 4:

(LC) Isocratic 15% A (20 mL/min flow rate). Mobile phase A was EtOH (200 proof), mobile phase B was HPLC grade heptane with 0.1% DEA. The column used for the chromatography was a Daicel IA, 20×250 mm column (5 μm particles).

### Method 5:

(LC) Isocratic 15% A (20 mL/min flow rate). Mobile phase A was EtOH (200 proof), mobile phase B was HPLC grade heptane with 0.125% DEA added. The column used for the chromatography was a Whelko R, R column (20×250 mm)

### Method 6:

(LC) Isocratic 20% A (20 mL/min flow rate). Mobile phase A was EtOH (200 proof), mobile phase B was HPLC grade heptane with 0.125% DEA added. The column used for the chromatography was a Daicel IA, 20×250 mm column (5 μm particles).

### Method 7:

(LC) Isocratic 30% A (20 mL/min flow rate). Mobile phase A was EtOH (200 proof), mobile phase B was HPLC grade heptane with 0.125% DEA added. The column used for the chromatography was a Whelko R, R column (20×250 mm)

### Method 8:

(LC) Isocratic 25% A (20 mL/min flow rate). Mobile phase A was EtOH (200 proof), mobile phase B was HPLC grade heptane with 0.12% DEA added. The column used for the chromatography was a Daicel IA, 20×250 mm column (5 μm particles).

### Method 9:

Gradient separation method wherein mobile phase A was EtOH (200 proof), mobile phase B was HPLC grade heptane with 0.12% DEA. Flow rate was 20 mL/min. Gradient was 10-22% A in 42 min, then ramp to 80% A in 0.5 min, hold at 59.5 min. The column used for the chromatography was a Regis Technologies, Whelk01 RR column (21×250 mm)

### Method 10:

Gradient separation method wherein mobile phase A was EtOH (200 proof), mobile phase B was HPLC grade heptane with 0.12% DEA. Flow rate was 20 mL/min. Gradient was 5-12% A in 25 min. The column used for the chromatography was a Regis Technologies, Whelk01 RR column (21×250 mm)

### Method 11:

Gradient separation method wherein mobile phase A was EtOH (200 proof), mobile phase B was HPLC grade heptane with 0.12% DEA. Flow rate was 20 mL/min. Gradient was 5-15% A in 31 min. The column used for the chromatography was a Daicel IB column (20×250 mm)

### Method 12:

Gradient separation method wherein mobile phase A was EtOH (200 proof), mobile phase B was HPLC grade heptane with 0.12% DEA. Flow rate was 20 mL/min. Gradient was 10-20% A in 40 min, then ramp to 70% A in 0.5 min, hold for 5.5 min. The column used for the chromatography was a Daicel IC column (20×250 mm)

### Method 13:

Gradient separation method wherein mobile phase A was EtOH (200 proof), mobile phase B was HPLC grade heptane with 0.12% DEA. Flow rate was 20 mL/min. Gradient was 10-20% A in 28 min, then ramp to 70% A in 0.5 min, hold for 1.5 min. The column used for the chromatography was a Daicel IC column (20×250 mm)

### Method 14:

Gradient separation method wherein mobile phase A was EtOH (200 proof), mobile phase B was HPLC grade heptane with 0.12% DEA. Flow rate was 20 mL/min. Gradient was 15% A for 15 min, step to 50% over 1 min, hold for 20 min. The column used for the chromatography was a Daicel 1C column (20×250 mm)

### Method 15:

Gradient separation method wherein mobile phase A was 2-propanol, mobile phase B was HPLC grade heptane with 0.12% DEA. Flow rate was 20 mL/min. Gradient was 10-16% A in 20 min, then ramp to 30% A in 1.0 min, hold for 6.0 min. The column used for the chromatography was a Daicel IA column (20×250 mm)

### Method 16:

Gradient separation method wherein mobile phase A was 2-propanol, mobile phase B was HPLC grade heptane with 0.12% DEA. Flow rate was 20 mL/min. Gradient was 2-11% A in 20 min, then hold at 11% A for 6.0 min. The column used for the chromatography was a Daicel IA column (20×250 mm)

### Method 17:

Gradient separation method wherein mobile phase A was EtOH (200 proof), mobile phase B was heptane with 0.12% DEA. Gradient was 10-50% A in 21 min then hold at 50% for 2 min (20 mL/min flow rate). The column used for the chromatography was a Daicel IA, 20×250 mm (5 μm particles).

### Method 18:

(LC) Isocratic 30% A (20 mL/min flow rate). Mobile phase A was EtOH (200 proof), mobile phase B was HPLC grade heptane with 0.1% DEA added. The column used for the chromatography was a Daicel IA, 20×250 mm column (5 μm particles).

### Method 19:

(LC) Isocratic 9% A (20 mL/min flow rate) for 23.5 min then step to 40% A in 0.5 min. Hold at 40% for 5 min. Mobile phase A was HPLC grade isopropanol, mobile phase B was HPLC grade heptane with 0.12% diethylamine added. The column used for the chromatography was a Daicel IA, 20×250 mm column (5 μm particles).

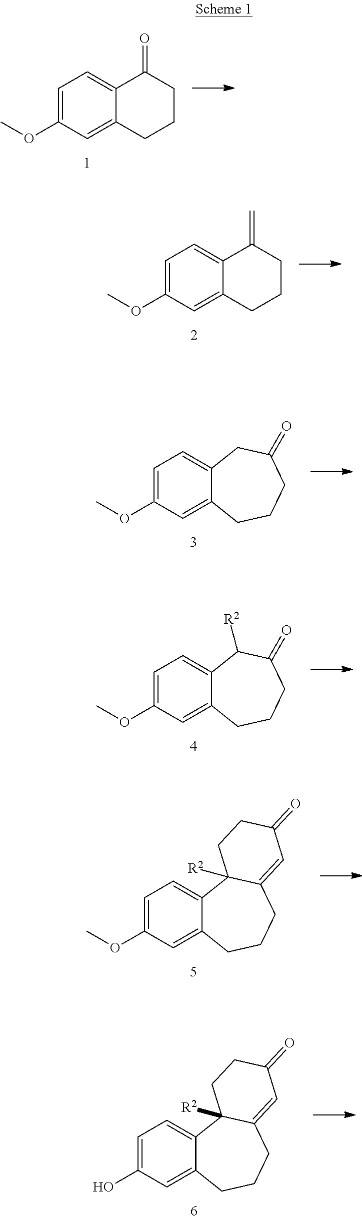

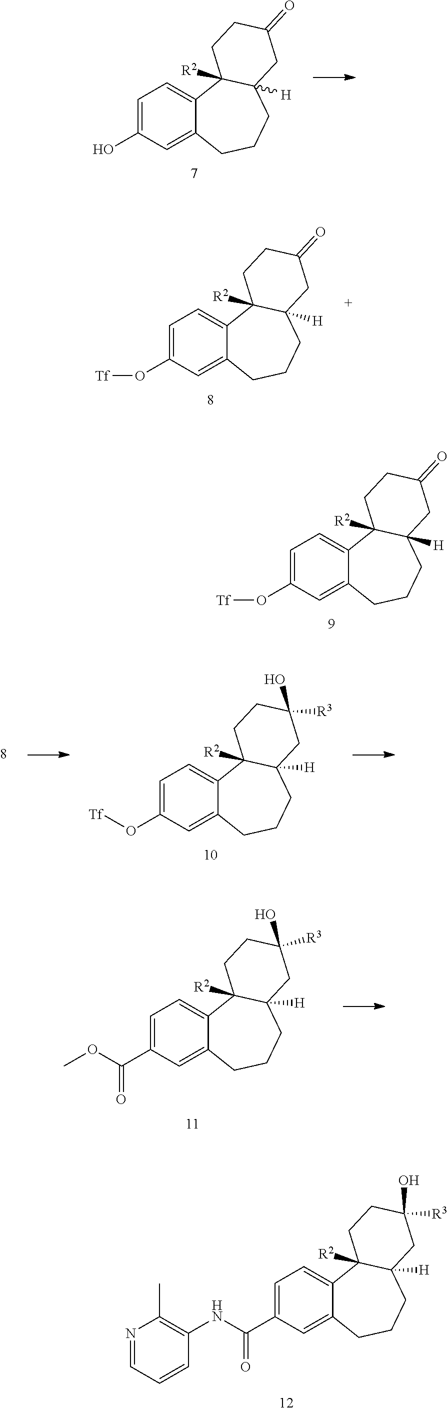

### Preparation #1: 5-Benzyl-2-methoxy-8,9-dihydro-5H-benzo[7]annulen-6(7H)-one (4, R²=Benzyl)

### Step #1: 6-Methoxy-1-methylene-1,2,3,4-tetrahydronaphthalene (2)

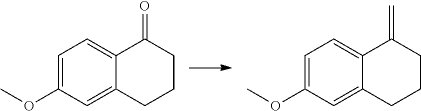

Step 1 was performed according to methods described by Michael W. Justik and Gerald F. Koser in Molecules 2005, 10, 217-225. Thus, into a 1 L 3-neck flask outfitted with a mechanical stirrer and addition funnel was added a suspension of sodium hydride (60% in mineral oil, 9.99 g, 250 mmol). The sodium hydride was washed with heptane (3×75 mL) and dry DMSO (163 mL) was added. The reaction was heated at about 60° C. for about 1 h and then cooled to rt. The reaction was diluted with THF (160 mL) and methyltriphenylphosphonium bromide (91.0 g, 256 mmol) was added in one portion. The reaction was stirred for about 30 min, then a solution of 6-methoxy-3,4-dihydronaphthalen-1(2H)-one (22.0 g, 125 mmol) in THF (85 mL) was added dropwise. The reaction was stirred for about 4 h, then poured into water (1000 mL) and extracted with Et₂O (3×500 mL). The combined Et₂O extracts were washed with water (500 mL), dried over Na₂SO₄, filtered and concentrated. The residue was extracted several times with 10% EtOAc in heptane (5×50 mL). The combined extracts were concentrated and the residue was purified on silica gel (200 g) using a gradient from 0-15% EtOAc in heptane. The product fractions were combined, concentrated and dried to constant weight to yield 6-methoxy-1-methylene-1,2,3,4-tetrahydronaphthalene (2) (21.5 g, 95%) as an oil. LC/MS, method 1, Rₜ=0.90 min, no parent ion. ¹H NMR (400 MHz, DMSO-d₆) δ 7.57 (d, J=8.7 Hz, 1H), 6.71 (dd, J=8.7, 2.8 Hz, 1H), 6.65 (d, J=2.7 Hz, 1H), 5.36 (d, J=1.1 Hz, 1H), 4.81 (d, J=1.4 Hz, 1H), 3.73 (s, 3H), 2.75 (t, J=6.2 Hz, 2H), 2.46-2.37 (m, 2H), 1.78-1.71 (m, 2H).

### Step #2: 2-Methnxv-8,9-dihydro-5H-benzo[7]annulen-6(7H)-one (3)

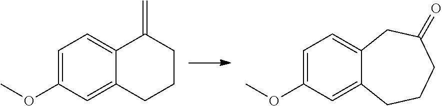

Step 2 was performed according to methods described by Michael W. Justik and Gerald F. Koser in Molecules 2005, 10, 217-225. Thus, a solution of 6-methoxy-1-methylene-1,2,3,4-tetrahydronaphthalene (2) (20.8 g, 119 mmol) in MeOH (200 mL) and water (10.4 mL) was cooled to about 0° C. and treated with [hydroxy(toslyoxy)iodo]benzene (46.7 g, 119 mmol) and the reaction was allowed to warm to rt. Water (250 mL) was added and the product was extracted with methylene chloride (2×250 mL). The residue was dried over Na₂SO₄, filtered and concentrated under reduced pressure. The residue was purified on silica gel (200 g) using a gradient from 0 to 15% EtOAc in heptane. Product fractions were combined and concentrated to yield 2-methoxy-8,9-dihydro-5H-benzo[7]annulen-6(7H)-one (3) as a viscous oil (19.7 g, 87%). LC/MS, method 1, Rₜ=0.66 min, MS m/z 191 (M+H)⁺. ¹H NMR (400 MHz, DMSO-d₆) δ 7.07 (d, J=8.2 Hz, 1H), 6.79 (d, J=2.7 Hz, 1H), 6.72 (dd, J=8.2, 2.7 Hz, 1H), 3.72 (s, 3H), 3.65 (s, 2H), 2.94-2.87 (m, 2H), 2.53-2.43 (m, 2H), 1.94-2.87 (m, 2H).

### Step #3: 5-Benzyl-2-methoxy-8,9-dihydro-5H-benzo[7]annulen-6(7H)-one (4, R²=Benzyl)

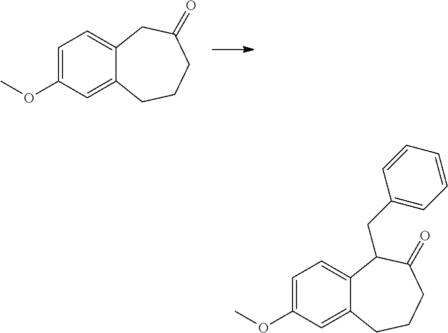

A solution of 2-methoxy-8,9-dihydro-5H-benzo[7]annulen-6(7H)-one (3) (19.5 g, 103 mmol) in toluene (400 mL) was treated with pyrrolidine (8.48 mL, 103 mmol) and the mixture was heated at reflux for about 2 h, removing water into a Dean-Stark trap. The reaction was cooled and concentrated, then re-dissolved in 1,4-dioxane (400 mL), treated with benzyl bromide (18.3 mL, 154 mmol) and heated at about 100° C. for about 18 h. The reaction was cooled, water (40 mL) was added and the mixture was heated at about 100° C. for about 2 h. The reaction was cooled and concentrated to about 100 mL, then distributed between EtOAc (400 mL) and water (400 mL). The organic layer was washed with 2N aqueous HCl (400 mL), then the combined aqueous layers were re-extracted with EtOAc (100 mL). The combined organic extracts were dried over Na₂SO₄, filtered and concentrated. The residue was purified on silica gel (330 g) using a gradient from 5 to 15% EtOAc in heptane. Product fractions were combined and concentrated. The residue was precipitated from EtOAc and heptane, filtered, rinsed with heptane and dried to yield 5-benzyl-2-methoxy-8,9-dihydro-5H-benzo[7]annulen-6(7H)-one (4, R²=Benzyl) as an off-white solid (16.2 g, 56%). LC/MS, method 1, Rₜ=0.88 min, MS m/z 281 (M+H)⁺. ¹H NMR (400 MHz, DMSO-d₆) δ 7.28-7.11 (m, 5H), 7.01 (d, J=8.5 Hz, 1H), 6.76 (d, J=2.7 Hz, 1H), 6.71 (dd, J=8.4, 2.8 Hz, 1H), 4.47-4.35 (m, 1H), 3.71 (s, 3H), 3.38 (dd, J=13.9, 8.3 Hz, 1H), 3.16-3.05 (m, 1H), 2.99 (dd, J=13.9, 6.3 Hz, 1H), 2.80-2.68 (m, 1H), 2.65-2.55 (m, 1H), 2.41-2.24 (m, 1H), 2.09-1.98 (m, 1H), 1.73-1.56 (m, 1H).

### Example #1

### 11b-Benzyl-9-methoxy-1,2,5,6,7,11b-hexahydro-dibenzo[a,c]cyclohepten-3-one (5, R²=Benzyl)

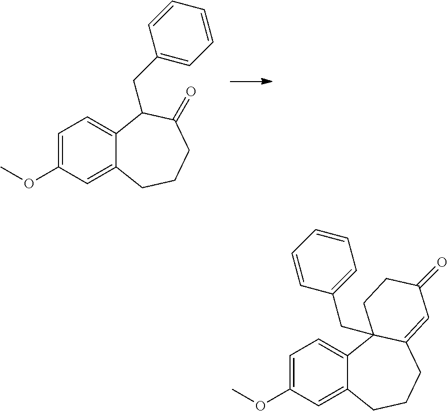

Freshly cut sodium (0.62 g, 26.8 mmol) was added in portions to EtOH (50 mL) under nitrogen and the mixture was stirred until the reaction was complete. A solution of 5-benzyl-2-methoxy-8,9-dihydro-5H-benzo[7]annulen-6(7H)-one (4, R²=Benzyl) (5.00 g, 17.8 mmol) in EtOH (50 mL) was added and the mixture was heated to about 60° C. Methyl vinyl ketone (1.47 mL, 17.8 mmol) was added dropwise over about 30 min, the reaction was heated at reflux for about 2.5 h, then cooled and concentrated. The residue was purified on silica gel (220 g) using a gradient from 10 to 35% EtOAc in heptane. Product fractions were combined and concentrated to about half volume. After standing about 4 h, 11b-benzyl-9-methoxy-1,2,5,6,7,11b-hexahydro-dibenzo[a,c]cyclohepten-3-one (5, R²=Benzyl) was collected by filtration and dried under vacuum, (4.04 g, 68%). LC/MS, method 1, Rₜ=0.88 min, MS m/z 333 (M+H)⁺. ¹H NMR (400 MHz, DMSO-d₆) δ 7.58 (d, J=8.8 Hz, 1H), 7.16-7.07 (m, 3H), 7.00 (m, 2H), 6.82 (dd, J=8.7, 2.9 Hz, 1H), 6.67 (d, J=2.9 Hz, 1H), 5.87 (s, 1H), 3.72 (s, 3H), 3.50 (d, J=13.5 Hz, 1H), 3.34 (d, J=13.5 Hz, 1H), 2.85-2.75 (m, 1H), 2.70-2.51 (m, 2H), 2.30-2.13 (m, 2H), 2.06-1.94 (m, 2H), 1.80-1.58 (m, 2H), 1.58-1.47 (m, 1H).

### Example #2

### 11b-Benzyl-9-hydroxy-1,2,5,6,7,11b-hexahydro-dibenzo[a,c]cyclohepten-3-one (6, R²=Benzyl)

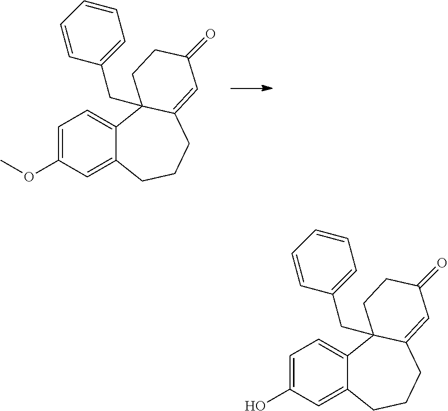

A mixture of 11b-benzyl-9-methoxy-1,2,5,6,7,11b-hexahydro-dibenzo[a,c]cyclohepten-3-one (5, R²=Benzyl) (3.00 g, 9.02 mmol) and DL-methinione (4.38 g, 29.3 mmol) in methansulfonic acid (30 mL) was allowed to stir under nitrogen at rt for about 48 h. The mixture was diluted with DCM (100 mL) and poured carefully onto ice water (100 mL). The product was extracted with DCM (2×100 mL), the combined organic layers were washed with water (100 mL), dried over Na₂SO₄, filtered and concentrated to solids. The residue was dried under vacuum to constant weight to yield 11b-benzyl-9-hydroxy-1,2,5,6,7,11b-hexahydro-dibenzo[a,c]cyclohepten-3-one (6, R²=Benzyl) as an off white solid (2.97 g, 103%-contained residual DCM). LC/MS, method 1, Rₜ=0.73 min, MS m/z 319 (M+H)⁺. ¹H NMR (400 MHz, DMSO-d₆) δ 9.23 (s, 1H), 7.43 (d, J=8.6 Hz, 1H), 7.15-7.06 (m, 3H), 7.05-6.97 (m, 2H), 6.64 (dd, J=8.6, 2.7 Hz, 1H), 6.49 (d, J=2.7 Hz, 1H), 5.85 (s, 1H), 3.45 (d, J=13.4 Hz, 1H), 3.33 (d, 1H), 2.79-2.67 (m, 1H), 2.66-2.55 (m, 1H), 2.49-2.39 (m, 1H), 2.33-2.14 (m, 2H), 2.03-1.90 (m, 2H), 1.74-1.48 (m, 3H).

### Example #3

### (7aR,9R,11aS)-11a-Benzyl-9-hydroxy-9-trifluoromethyl-6,7,7a,8,9,10,11,11a-octahydro-5H-dibenzo[a,c]cycloheptene-3-carboxylic acid (2-methyl-pyridin-3-yl)-amide; compound with (7aS,9S,11aR)-11a-benzyl-9-hydroxy-9-trifluoromethyl-6,7,7a,8,9,10,11,11a-octahydro-5H-dibenzo[a,c]cycloheptene-3-carboxylic acid (2-methyl-pyridin-3-yl)-amide (12, R²=Benzyl, R³=Trifluoromethyl)

### Step #1: 11b-Benzyl-9-hydroxy-1,2,4,4a,5,6,7,11b-octahydro-dibenzo[a,c]cyclohepten-3-one (7, R²=Benzyl)

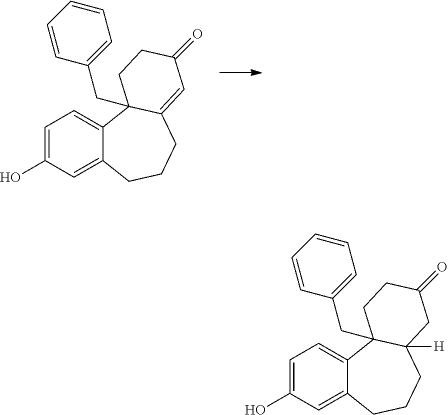

A mixture of 11b-benzyl-9-hydroxy-1,2,5,6,7,11b-hexahydro-dibenzo[a,c]cyclohepten-3-one (6, R²=Benzyl) (5.90 g, 18.5 mmol) and 20% Pd(OH)₂ on carbon (1.30 g) in toluene (111 mL) was hydrogenated in a Pan Shaker at about 50° C. under about 60 psi hydrogen for about 20 h. The reaction was filtered through a pad of Celite® (about 5.0 g) to remove the catalyst. The Celite® pad was washed with EtOAc (2×220 mL). The combined filtrates were combined and concentrated to yield 11b-benzyl-9-hydroxy-1,2,4,4a,5,6,7,11b-octahydro-dibenzo[a,c]cyclohepten-3-one (7, R²=Benzyl) (4.95 g, 82%) as a mixture of isomers which was taken to the next step without further purification.

### Step #2: Trifluoro-methanesulfonic acid (7aR,11aS)-11a-benzyl-9-oxo-6,7,7a,8,9,10,11,11a-octahydro-5H-dibenzo[a,c]cyclohepten-3-yl ester; compound with trifluoro-methanesulfonic acid (7aS,11aR)-11a-benzyl-9-oxo-6,7,7a,8,9,10,11,11a-octahydro-5H-dibenzo[a,c]cyclohepten-3-yl ester (8, R²=Benzyl) and trifluoro-methanesulfonic acid (7aS,11aS)-11a-benzyl-9-oxo-6,7,7a,8,9,10,11,11a-octahydro-5H-dibenzo[a,c]cyclohepten-3-yl ester; compound with trifluoro-methanesulfonic acid (7aR,11aR)-11a-benzyl-9-oxo-6,7,7a,8,9,10,11,11a-octahydro-5H-dibenzo[a,c]cyclohepten-3-yl ester (9, R²=Benzyl)

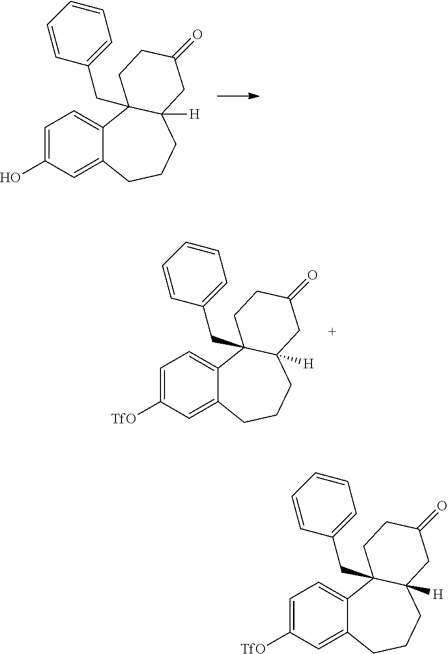

A slurry of 11b-benzyl-9-hydroxy-1,2,4,4 a,5,6,7,11b-octahydro-dibenzo[a,c]cyclohepten-3-one (7, R²=Benzyl) (25.93 g, 80.9 mmol) in DCM (570 mL) was treated with N-phenylbis(trifluoromethanesulfonimide) (29.0 g, 80.9 mmol) and DIEA (28.3 mL, 162 mmol) at rt. The reaction was stirred for about 17 h, then silica gel (350 g) was added and the mixture was concentrated to dryness. The residue was divided in two portions and each portion was loaded separately in a cartridge and purified on silica gel (330 g) using a gradient from 10-30% EtOAc in heptane. The pure products were collected separately. The mixed fractions from each column were combined and re-purified on a third column (330 g) using the conditions described above to provide a combined yield of trifluoro-methanesulfonic acid (7aR,11aS)-11a-benzyl-9-oxo-6,7,7a,8,9,10,11,11a-octahydro-5H-dibenzo[a,c]cyclohepten-3-yl ester; compound with trifluoro-methanesulfonic acid (7aS,11aR)-11a-benzyl-9-oxo-6,7,7a,8,9,10,11,11a-octahydro-5H-dibenzo[a,c]cyclohepten-3-yl ester (8, R²=Benzyl) (9.78 g, 26%). LC/MS, method 2, Rₜ, =2.94 min, no parent ion. ¹H NMR (400 MHz, DMSO-d₆) δ 7.36 (m, 1H), 7.11-6.96 (m, 5H), 6.57-6.52 (m, 2H), 3.60-2.96 (d, J=14.0 Hz, 1H), 3.51-3.41 (m, 1H), 3.17 (d, J=13.9 Hz, 1H), 3.06-2.96 m, 1H), 2.90-2.74 (m, 1H), 2.74-2.63 (m, 1H), 2.24-2.14 (m, 1H), 2.14-1.95 (m, 5H), 1.95-1.82 (m, 1H), 1.74-1.62 (m, 1H), 1.47-1.34 (m, 1H) and trifluoro-methanesulfonic acid (7aS,11aS)-11a-benzyl-9-oxo-6,7,7a,8,9,10,11,11a-octahydro-5H-dibenzo[a,c]cyclohepten-3-yl ester; compound with trifluoro-methanesulfonic acid (7aR,11aR)-11a-benzyl-9-oxo-6,7,7a,8,9,10,11,11a-octahydro-5H-dibenzo[a,c]cyclohepten-3-yl ester (9, R²=Benzyl) (15.8 g, 43%). LC/MS, method 2, Rₜ=2.98 min; MS m/z: no parent ion. ¹H NMR (400 MHz, DMSO-d₆) δ 7.39 (d, J=2.9 Hz, 1H), 7.11-6.99 (m, 4H), 6.93 (d, J=9.0 Hz, 1H), 6.53-6.47 (m, 2H), 3.66 (d, J=13.1 Hz, 1H), 3.32-3.25 (m, 1H), 3.02 (dd, J=15.4, 5.4 Hz, 1H), 2.59 (d, J=13.2 Hz, 1H), 2.46-2.05 (m, 6H), 2.05-1.84 (m, 2H), 1.84-1.74 (m, 1H), 1.74-1.62 (m, 1H), 1.62-1.47 (m, 1H), each as an off white solid.

### Step #3: Trifluoro-methanesulfonic acid (7aR,9R,11aS)-11a-benzyl-9-hydroxy-9-trifluoromethyl-6,7,7a,8,9,10,11,11a-octahydro-5H-dibenzo[a,c]cyclohepten-3-yl ester; compound with trifluoro-methanesulfonic acid (7aS,9S,11aR)-11a-benzyl-9-hydroxy-9-trifluoromethyl-6,7,7a,8,9,10,11,11a-octahydro-5H-dibenzo[a,c]cyclohepten-3-yl ester (10, R²=Benzyl, R³=Trifluoromethyl)

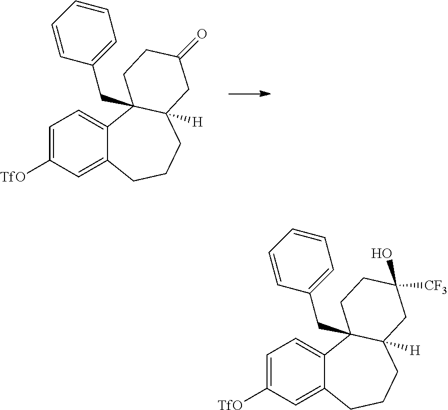

A solution of trifluoromethanesulfonic acid (7aR,11aS)-11a-benzyl-9-oxo-6,7,7a,8,9,10,11,11a-octahydro-5H-dibenzo[a,c]cyclohepten-3-yl ester; compound with trifluoro-methanesulfonic acid (7aS,11aR)-11a-benzyl-9-oxo-6,7,7a,8,9,10,11,11a-octahydro-5H-dibenzo[a,c]cyclohepten-3-yl ester (8, R²=Benzyl) (0.320 g, 0.707 mmol) in DCM (15 mL) was cooled to about 0° C. under nitrogen. TBAF (1M solution in THF) (7 μL, 7 μmol) was added and then (trifluoromethyl)trimethylsilane (0.157 mL, 1.06 mmol) was added dropwise over about 20 min. The reaction was allowed to warm slowly to rt. The reaction was re-cooled to about 0° C., (trifluoromethyl)trimethylsilane (0.157 mL, 1.06 mmol) was added and then 2 drops of TBAF were added. The addition of reagents was repeated several times, then the reaction was allowed to warm to rt and concentrated. The residue was dissolved in DCM (15 mL), cooled to about 0° C. and TBAF (0.707 mL, 0.707 mmol) was added dropwise. The mixture was stirred for about 30 min then washed with water (2×15 mL). The residue was dried over Na₂SO₄, filtered and concentrated. The residue was purified on silica gel (40 g) using a gradient from 10 to 25% EtOAc in heptane. Product fractions were combined and concentrated to yield trifluoro-methanesulfonic acid (7aR,9R,11aS)-11a-benzyl-9-hydroxy-9-trifluoromethyl-6,7,7a,8,9,10,11,11a-octahydro-5H-dibenzo[a,c]cyclohepten-3-yl ester; compound with trifluoro-methanesulfonic acid (7aS,9S,11aR)-11a-benzyl-9-hydroxy-9-trifluoromethyl-6,7,7a,8,9,10,11,11a-octahydro-5H-dibenzo[a,c]cyclohepten-3-yl ester (10, R²=Benzyl, R³=Trifluoromethyl) (0.160 g, 43%) as an off-white solid. LC/MS, method 1, Rₜ=1.03 min, MS m/z 581 (M+OAc)⁻. ¹H NMR (400 MHz, DMSO-d₆) δ 7.35 (d, J=2.0 Hz, 1H), 7.08-6.94 (m, 5H), 6.48-6.42 (m, 2H), 5.92 (s, 1H), 3.49 (d, J=13.5 Hz, 1H), 3.48-3.36 (m, 1H), 2.99 (dd, J=15.0, 5.3 Hz, 1H), 2.88 (d, J=13.7 Hz, 1H), 2.06-1.63 (m, 10H), 1.50-1.35 (m, 1H).

### Step #4: (7aR,9R,11aS)-11a-Benzyl-9-hydroxy-9-trifluoromethyl-6,7,7a,8,9,10,11,11a-octahydro-5H-dibenzo[a,c]cycloheptene-3-carboxylic acid methyl ester; compound with (7aS,9S,11aR)-11a-benzyl-9-hydroxy-9-trifluoromethyl-6,7,7a,8,9,10,11,11a-octahydro-5H-dibenzo[a,c]cycloheptene-3-carboxylic acid methyl ester (11, R²=Benzyl, R³=Trifluoromethyl)

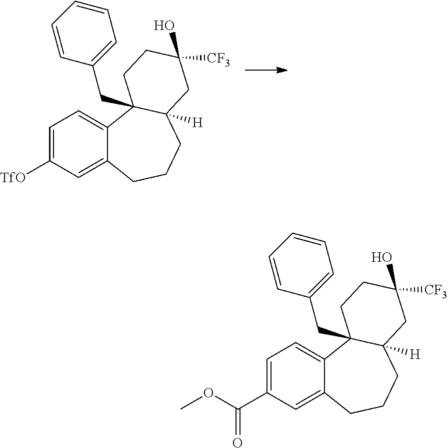

To a mixture of trifluoromethanesulfonic acid (7aR,9R,11aS)-11a-benzyl-9-hydroxy-9-trifluoromethyl-6,7,7a,8,9,10,11,11a-octahydro-5H-dibenzo[a,c]cyclohepten-3-yl ester; compound with trifluoro-methanesulfonic acid (7aS,9S,11aR)-11a-benzyl-9-hydroxy-9-trifluoromethyl-6,7,7a,8,9,10,11,11a-octahydro-5H-dibenzo[a,c]cyclohepten-3-yl ester (10, R²=Benzyl, R³=Trifluoromethyl) (0.155 g, 0.297 mmol), Xantphos (0.017 g, 0.030 mmol) and Pd₂(dba)₃ (0.008 g, 9 μmol) was added DMF (1.5 mL) and the mixture was degassed using a stream of nitrogen. The reaction vessel was briefly evacuated and an atmosphere of carbon monoxide was introduced via balloon. To the mixture was added MeOH (0.072 mL, 1.8 mmol) and TEA (0.083 mL, 0.59 mmol) and the reaction was heated at about 100° C. for about 4 h. The reaction was cooled to rt and concentrated. The residue was purified on silica gel (40 g) using a gradient from 20-75% EtOAc in heptane. Product fractions were combined, concentrated and dried under vacuum to yield (7aR,9R,11aS)-11a-benzyl-9-hydroxy-9-trifluoromethyl-6,7,7a,8,9,10,11,11a-octahydro-5H-dibenzo[a,c]cycloheptene-3-carboxylic acid methyl ester; compound with (7aS,9S,11aR)-11a-benzyl-9-hydroxy-9-trifluoromethyl-6,7,7a,8,9,10,11,11a-octahydro-5H-dibenzo[a,c]cycloheptene-3-carboxylic acid methyl ester (11, R²=Benzyl, R³=Trifluoromethyl) (0.072 g, 56%) as an off white solid. LC/MS, method 1, Rₜ=0.96 min, MS m/z 491 (M+OAc)⁻. ¹H NMR (400 MHz, DMSO-d₆) δ 7.79 (d, J=2.1 Hz, 1H), 7.55 (dd, J=8.3, 2.0 Hz, 1H), 7.08-6.97 (m, 4H), 6.51 (dd, J=7.5, 1.9 Hz, 2H), 5.92 (s, 1H), 3.83 (s, 3H), 3.56 (d, J=13.8 Hz, 1H), 3.50-3.39 (m, 1H), 3.00 (dd, J=15.0, 5.0 Hz, 1H), 2.88 (d, J=13.7 Hz, 1H), 2.09-1.63 (m, 10H), 1.47-1.34 (m, 1H).

### Step #5: (7aR,9R,11aS)-11a-Benzyl-9-hydroxy-9-trifluoromethyl-6,7,7a,8,9,10,11,11a-octahydro-5H-dibenzo[a,c]cycloheptene-3-carboxylic acid (2-methyl-pyridin-3-yl)-amide; compound with (7aS,9S,11aR)-11a-benzyl-9-hydroxy-9-trifluoromethyl-6,7,7a,8,9,10,11,11a-octahydro-5H-dibenzo[a,c]cycloheptene-3-carboxylic acid (2-methyl-pyridin-3-yl)-amide (12, R²=Benzyl, R³=Trifluoromethyl)

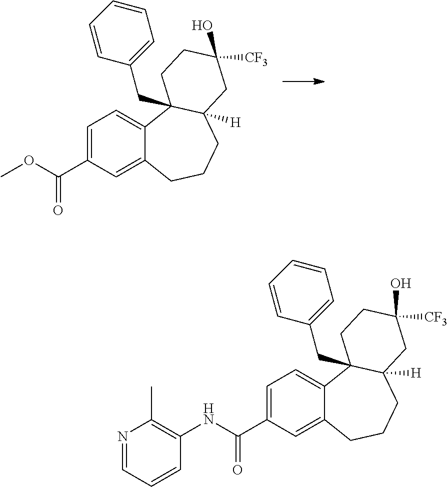

A solution of (7aR,9R,11aS)-11a-benzyl-9-hydroxy-9-trifluoromethyl-6,7,7a,8,9,10,11,11a-octahydro-5H-dibenzo[a,c]cycloheptene-3-carboxylic acid methyl ester; compound with (7aS,9S,11aR)-11a-benzyl-9-hydroxy-9-trifluoromethyl-6,7,7a,8,9,10,11,11a-octahydro-5H-dibenzo[a,c]cycloheptene-3-carboxylic acid methyl ester (11, R²=Benzyl, R³=Trifluoromethyl) (0.070 g, 0.16 mmol) and 3-amino-2-methylpyridine (0.018 g, 0.17 mmol) in toluene (1.5 mL) was stirred at rt under nitrogen and LiHMDS (0.470 mL, 0.470 mmol) (1M solution in THF) was added dropwise. The mixture was stirred for about 30 min, quenched with water (2 mL) and the crude product was extracted with EtOAc (2×5 mL). The combined organic layers were dried over Na₂SO₄, filtered and concentrated. The residue was purified on silica gel (12 g) using a gradient from 80 to 100% EtOAc in heptane. Pure product fractions were combined and concentrated to an oil that was precipitated from MeCN with water. The product was filtered off and dried under vacuum to yield (7aR,9R,11aS)-11a-benzyl-9-hydroxy-9-trifluoromethyl-6,7,7a,8,9,10,11,11a-octahydro-5H-dibenzo[a,c]cycloheptene-3-carboxylic acid (2-methyl-pyridin-3-yl)-amide; compound with (7aS,9S,11aR)-11a-benzyl-9-hydroxy-9-trifluoromethyl-6,7,7a,8,9,10,11,11a-octahydro-5H-dibenzo[a,c]cycloheptene-3-carboxylic acid (2-methyl-pyridin-3-yl)-amide (12, R²=Benzyl, R³=Trifluoromethyl) (0.033 g, 41%) as an off-white solid. LC/MS, method 1, Rₜ=0.74 min, MS m/z 509 (M+H)⁺. ¹H NMR (400 MHz, DMSO-d₆) δ 9.94 (s, 1H), 8.31 (dd, J=4.8, 1.6 Hz, 1H), 7.81 (d, J=2.0 Hz, 1H), 7.72 (dd, J=8.0, 1.5 Hz, 1H), 7.59 (dd, J=8.4, 1.9 Hz, 1H), 7.25 (dd, J=7.9, 4.8 Hz, 1H), 7.12-6.93 (m, 4H), 6.56 (dd, J=6.5, 2.9 Hz, 2H), 5.90 (s, 1H), 3.58 (d, J=13.5 Hz, 1H), 3.54-3.44 (m, 1H), 3.06-2.96 (m, 1H), 2.87 (d, J=13.8 Hz, 1H), 2.42 (s, 3H), 2.11-1.59 (m, 10H), 1.53-1.39 (m, 1H).

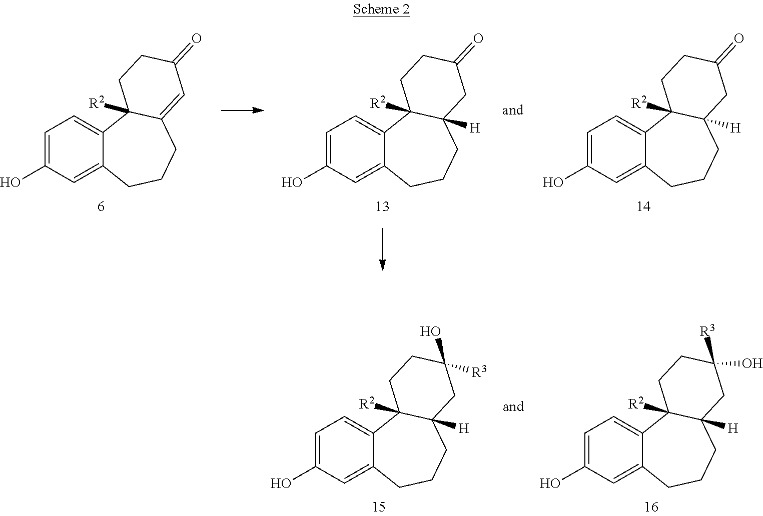

### Examples #4 and 5

### (4aS,11bS)-11b-Benzyl-9-hydroxy-1,2,4,4a,5,6,7,11b-octahydro-dibenzo[a,c]cyclohepten-3-one; compound with (4aR,11bR)-11b-benzyl-9-hydroxy-1,2,4,4a,5,6,7,11b-octahydro-dibenzo[a,c]cyclohepten-3-one (13, R²=Benzyl) and (4aR,11bS)-11b-benzyl-9-hydroxy-1,2,4,4a,5,6,7,11b-octahydro-dibenzo[a,c]cyclohepten-3-one; compound with (4aS,11bR)-11b-benzyl-9-hydroxy-1,2,4,4a,5,6,7,11b-octahydro-dibenzo[a,c]cyclohepten-3-one (14, R²=Benzyl)

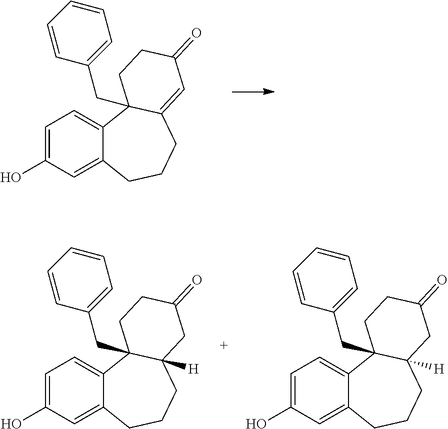

A mixture of 11b-benzyl-9-hydroxy-1,2,5,6,7,11b-hexahydro-dibenzo[a,c]cyclohepten-3-one (6, R²=Benzyl) (0.250 g, 0.785 mmol) and 20% Pd(OH)₂ on carbon (0.055 g) in toluene (3 mL) and 1,4-dioxane (3 mL) was shaken under about 40 psi hydrogen at about 60° C. for about 18 h. The reaction was cooled to rt and filtered through Celite®, rinsing with EtOAc. The filtrate was concentrated to an oil, then purified on C18 using a gradient 25-65% MeCN/50 mM NH₄OAc buffer. The first peak and the second peak were isolated separately. Each product precipitated from solution as a white solid on concentration. Each product was filtered, washed with water (2 mL) and dried under vacuum to yield (4aS,11bS)-11b-benzyl-9-hydroxy-1,2,4,4a,5,6,7,11b-octahydro-dibenzo[a,c]cyclohepten-3-one; compound with (4aR,11bR)-11b-benzyl-9-hydroxy-1,2,4,4a,5,6,7,11b-octahydro-dibenzo[a,c]cyclohepten-3-one (13, R²=Benzyl) (0.054 g, 21%) as a white solid, LC/MS, method 1, Rₜ=0.78 min, MS m/z 319 (M−H)⁻, ¹H NMR (400 MHz, DMSO-d₆) δ 9.13 (s, 1H), 7.06-6.97 (m, 3H), 6.68 (d, J=8.8 Hz, 1H), 6.63-6.59 (m, 3H), 6.34 (dd, J=8.7, 2.8 Hz, 1H), 3.58-3.49 (m, 1H), 3.38-3.27 (m, 1H), 3.08 (d, J=13.7 Hz, 1H), 2.84-2.69 (m, 2H), 2.69-2.59 (m, 1H), 2.16-1.85 (m, 6H), 1.85-1.71 (m, 1H), 1.70-1.59 (m, 1H), 1.43-1.29 (m, 1H) and (4aR,11bS)-11b-benzyl-9-hydroxy-1,2,4,4a,5,6,7,11b-octahydro-dibenzo[a,c]cyclohepten-3-one; compound with (4aS,11bR)-11b-benzyl-9-hydroxy-1,2,4,4a,5,6,7,11b-octahydro-dibenzo[a,c]cyclohepten-3-one (14, R²=Benzyl) (0.089 g, 35%) as a white solid, LC/MS, method 1, Rₜ=0.80 min, MS m/z 319 (M−H)⁻. ¹H NMR (400 MHz, DMSO-d₆) δ 9.15 (s, 1H), 7.10-6.99 (m, 3H), 6.63 (d, J=2.7 Hz, 1H), 6.59-6.48 (m, 3H), 6.37 (dd, J=8.6, 2.7 Hz, 1H), 3.60 (d, J=12.9 Hz, 1H), 3.22-3.10 (m, 1H), 2.74 (dd, J=14.6, 5.0 Hz, 1H), 2.55-2.45 (m, 1H), 2.40-2.07 (m, 6H), 1.89 (d, J=13.9 Hz, 1H), 1.79-1.70 (m, 2H), 1.67-1.60 (m, 1H), 1.56-1.45 (m, 1H).

### Examples #6 and 7

### (3R,4aS,11bS)-11b-Benzyl-3-methyl-2,3,4,4a,5,6,7,11b-octahydro-1H-dibenzo[a,c]cycloheptene-3,9-diol; compound with (3S,4aR,11bR)-11b-benzyl-3-methyl-2,3,4,4a,5,6,7,11b-octahydro-1H-dibenzo[a,c]cycloheptene-3,9-diol (15, R²=Benzyl, R³=Methyl) and (3R,4aR,11bR)-11b-benzyl-3-methyl-2,3,4,4a,5,6,7,11b-octahydro-1H-dibenzo[a,c]cycloheptene-3,9-diol; compound with (3S,4aS,11bS)-11b-benzyl-3-methyl-2,3,4,4a,5,6,7,11b-octahydro-1H-dibenzo[a,c]cycloheptene-3,9-diol (16, R²=Benzyl, R³=Methyl)

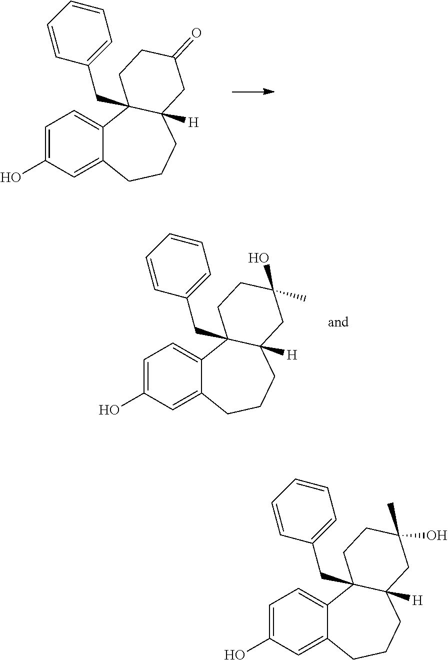

To a stirred solution of methylmagnesium bromide (3 M solution in Et₂O, 0.520 mL, 1.56 mmol) at about 0° C. was added a solution of (4aS,11bS)-11b-benzyl-9-hydroxy-1,2,4,4a,5,6,7,11b-octahydro-dibenzo[a,c]cyclohepten-3-one; compound with (4 aR,11bR)-11b-benzyl-9-hydroxy-1,2,4,4 a,5,6,7,11b-octahydro-dibenzo[a,c]cyclohepten-3-one (13, R²=Benzyl) (0.050 g, 0.16 mmol) in Et₂O (1.00 mL) and THF (3 mL) dropwise. The mixture was stirred about 20 min under nitrogen at about 0° C. and then allowed to warm to rt with stirring for about an additional 1 h. Water (10 mL) was added dropwise and then THF was removed under reduced pressure. Crude product was extracted with DCM (3×10 mL). The organics were dried over Na₂SO₄, filtered and concentrated. The residue was purified on C18 using a gradient from 25 to 40% MeCN in 50 mM aqueous NH₄OAc buffer. The first peak and the second peak were isolated separately. Each product precipitated from solution as a white solid on concentration. Separately, each product was collected by filtration, washed with water (2.0 mL) and dried under vacuum to yield (3R,4aS,11bS)-11b-benzyl-3-methyl-2,3,4,4a,5,6,7,11b-octahydro-1H-dibenzo[a,c]cycloheptene-3,9-diol; compound with (3S,4aR,11bR)-11b-benzyl-3-methyl-2,3,4,4a,5,6,7,11b-octahydro-1H-dibenzo[a,c]cycloheptene-3,9-diol (15, R²=Benzyl, R³=Methyl) (0.023 g, 44%) as a white solid. LC/MS, method 1, Rₜ=0.76 min, MS m/z 319 (M−OH)⁺. ¹H NMR (400 MHz, DMSO-d₆) δ 9.01 (s, 1H), 7.06-7.00 (m, 3H), 6.59-6.55 (m, 1H), 6.55-6.48 (m, 2H), 6.41 (d, J=8.7 Hz, 1H), 6.30 (dd, J=8.6, 2.7 Hz, 1H), 3.99 (s, 1H), 3.48 (d, J=12.7 Hz, 1H), 3.16-3.06 (m, 1H), 2.72 (dd, J=14.7, 5.6 Hz, 1H), 2.48-2.39 (m, 1H), 2.40-2.25 (m, 2H), 1.77-1.26 (m, 7H), 1.23-1.18 (m, 1H), 1.09 (d, J=12.9 Hz, 1H), 0.93 (s, 3H), and (3R,4aR,11bR)-11b-benzyl-3-methyl-2,3,4,4a,5,6,7,11b-octahydro-1H-dibenzo[a,c]cycloheptene-3,9-diol; compound with (3S,4aS,11bS)-11b-benzyl-3-methyl-2,3,4,4a,5,6,7,11b-octahydro-1H-dibenzo[a,c]cycloheptene-3,9-diol (16, R²=Benzyl, R³=Methyl) (0.008 g, 10%) as a white solid. LC/MS, method 1, Rₜ=0.82 min, MS m/z 319 (M−OH)⁺, ¹H NMR (400 MHz, DMSO-d₆) δ 9.02 (s, 1H), 7.07-6.93 (m, 3H), 6.60-6.29 (m, 5H), 4.10 (s, 1H), 3.51-3.41 (m, 1H), 3.17-3.04 (m, 1H), 2.80-2.63 (m, 1H), 2.49-2.37 (m, 1H), 2.39-2.27 (m, 1H), 1.93-1.77 (m, 2H), 1.76-1.28 (m, 7H), 1.17 (s, 3H), 1.11-1.02 (m, 1H).

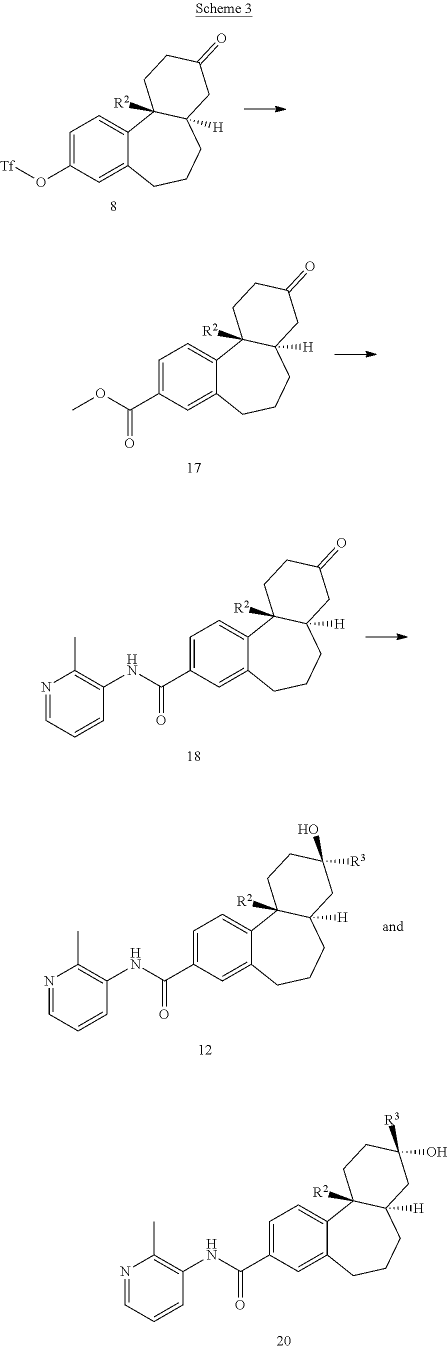

### Example #8

### (7aR,11aS)-11a-Benzyl-9-oxo-6,7,7a,8,9,10,11,11a-octahydro-5H-dibenzo[a,c]cycloheptene-3-carboxylic acid (2-methyl-pyridin-3-yl)-amide; compound with (7aS,11aR)-11a-benzyl-9-oxo-6,7,7a,8,9,10,11,11a-octahydro-5H-dibenzo[a,c]cycloheptene-3-carboxylic acid (2-methyl-pyridin-3-yl)-amide (18, R²=Benzyl)

### Step #1: (7aR,11aS)-11a-Benzyl-9-oxo-6,7,7a,8,9,10,11,11a-octahydro-5H-dibenzo[a,c]cycloheptene-3-carboxylic acid methyl ester; compound with (7aS,11aR)-11a-benzyl-9-oxo-6,7,7a,8,9,10,11,11a-octahydro-5H-dibenzo[a,c]cycloheptene-3-carboxylic acid methyl ester (17, R²=Benzyl)

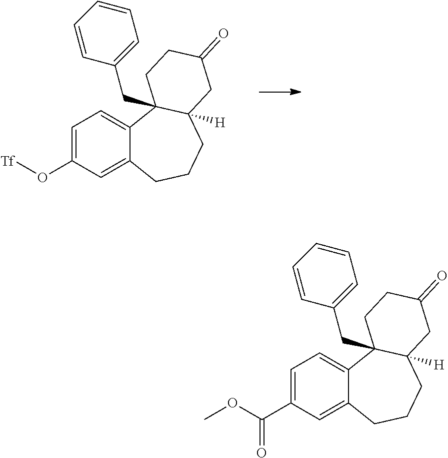

A solution of trifluoromethanesulfonic acid (7aR,11aS)-11a-benzyl-9-oxo-6,7,7a,8,9,10,11,11a-octahydro-5H-dibenzo[a,c]cyclohepten-3-yl ester; compound with trifluoro-methanesulfonic acid (7aS,11aR)-11a-benzyl-9-oxo-6,7,7a,8,9,10,11,11a-octahydro-5H-dibenzo[a,c]cyclohepten-3-yl ester (8, R²=Benzyl) (3.20 g, 7.07 mmol) in DMF (20 mL) was treated with Xantphos (0.409 g, 0.707 mmol) and Pd₂(dba)₃ (0.194 g, 0.212 mmol). The reaction vessel was evacuated and an atmosphere of carbon monoxide was introduced. TEA (1.97 mL, 14.1 mmol) and MeOH (1.72 mL, 42.4 mmol) were added. The mixture was heated at about 100° C. for about 48 h. The mixture was allowed to cool to rt then concentrated under reduced pressure. The residue was taken up in EtOAc (50 mL) and washed with saturated aqueous NaHCO₃. The organic layer was dried over Na₂SO₄, filtered and concentrated. The residue was purified on silica gel (120 g) using a gradient from 15 to 40% EtOAc in heptane. Product fractions were combined, concentrated and dried under vacuum to yield (7aR,11aS)-11a-benzyl-9-oxo-6,7,7a,8,9,10,11,11a-octahydro-5H-dibenzo[a,c]cycloheptene-3-carboxylic acid methyl ester; compound with (7aS,11aR)-11a-benzyl-9-oxo-6,7,7a,8,9,10,11,11a-octahydro-5H-dibenzo[a,c]cycloheptene-3-carboxylic acid methyl ester (17, R²=Benzyl) as a colorless oil. LC/MS, method 3, Rₜ=1.78 min, no parent ion. ¹H NMR (400 MHz, DMSO-d₆) δ 7.78 (d, J=2.1 Hz, 1H), 7.55 (dd, J=8.4, 2.0 Hz, 1H), 7.06 (d, J=8.6 Hz, 1H), 7.03-6.95 (m, 3H), 6.57 (dd, J=7.6, 1.8 Hz, 2H), 3.82 (s, 3H), 3.65 (d, J=13.8 Hz, 1H), 3.55-3.44 (m, 1H), 3.17 (d, J=13.7 Hz, 1H), 3.07-2.98 (m, 1H), 2.87-2.76 (m, 1H), 2.74-2.63 (m, 1H), 2.23-2.14 (m, 1H), 2.15-1.99 (m, 5H), 1.96-1.85 (m, 1H), 1.73-1.63 (m, 1H), 1.45-1.33 (m, 1H).

### Step #2: (7aR,11aS)-11a-Benzyl-9-oxo-6,7,7a,8,9,10,11,11a-octahydro-5H-dibenzo[a,c]cycloheptene-3-carboxylic acid (2-methyl-pyridin-3-yl)-amide; compound with (7aS,11aR)-11a-benzyl-9-oxo-6,7,7a,8,9,10,11,11a-octahydro-5H-dibenzo[a,c]cycloheptene-3-carboxylic acid (2-methyl-pyridin-3-yl)-amide (18, R²=Benzyl)

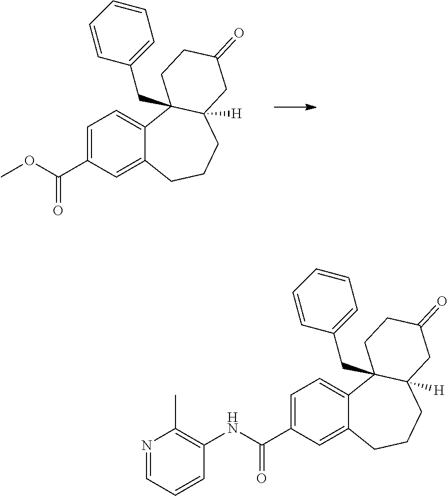

A solution of (7aR,11aS)-11a-benzyl-9-oxo-6,7,7a,8,9,10,11,11a-octahydro-5H-dibenzo[a,c]cycloheptene-3-carboxylic acid methyl ester; compound with (7aS,11aR)-11a-benzyl-9-oxo-6,7,7a,8,9,10,11,11a-octahydro-5H-dibenzo[a,c]cycloheptene-3-carboxylic acid methyl ester (17, R₂=Benzyl) (0.750 g, 2.07 mmol) in 1,4-dioxane (12 mL) was treated with LiOH (0.261 g, 6.21 mmol) and water (3 mL). The reaction was warmed briefly to about 50° C., then diluted with water to obtain a homogeneous solution. The conversion to acid was followed to completion by LC/MS (LC/MS method 3, Rₜ=1.37 min, MS m/z 347 (M−H)⁻. The mixture was acidified with 2N aqueous HCl (20 mL) and extracted with EtOAc (2×20 mL). The extracts were dried over Na₂SO₄, filtered and concentrated. The residue was dissolved in THF (25 mL), DIEA (0.367 mL, 2.10 mmol) was added and the mixture was treated with TFFH (0.556 g, 2.10 mmol) at rt for about 5 min, and then with 2-methylpyridin-3-amine (0.455 g, 4.21 mmol). The reaction was stirred for about 48 h at about 60° C. The reaction was cooled and concentrated under reduced pressure. The residue was dissolved in DCM (60 mL) and washed with saturated aqueous NaHCO₃ (30 mL), dried over Na₂SO₄, filtered and concentrated under reduced pressure. The residue was purified on silica gel (80 g) using a gradient from 80 to 100% EtOAc in heptane. The product fractions were combined, concentrated under reduced pressure and dried under vacuum to yield (7aR,11aS)-11a-benzyl-9-oxo-6,7,7a,8,9,10,11,11a-octahydro-5H-dibenzo[a,c]cycloheptene-3-carboxylic acid (2-methyl-pyridin-3-yl)-amide; compound with (7aS,11aR)-11a-benzyl-9-oxo-6,7,7a,8,9,10,11,11a-octahydro-5H-dibenzo[a,c]cycloheptene-3-carboxylic acid (2-methyl-pyridin-3-yl)-amide (18, R²=Benzyl) (0.525 g, 57%) as an off-white glass. LC/MS, method 2, Rₜ=2.15 min, MS m/z 439 (M+H)⁺. ¹H NMR (400 MHz, DMSO-d₆) δ 10.01-9.95 (m, 1H), 8.36-8.29 (m, 1H), 7.85-7.82 (m, 1H), 7.76-7.70 (m, 1H), 7.64-7.60 (m, 1H), 7.30-7.24 (m, 1H), 7.12-7.00 (m, 4H), 6.68-6.62 (m, 2H), 3.75-3.65 (m, 1H), 3.59-3.47 (m, 1H), 3.23-3.15 (m, 1H), 3.08-2.98 (m, 1H), 2.90-2.71 (m, 2H), 2.44 (s, 3H), 2.23-1.89 (m, 7H), 1.75-1.64 (m, 1H), 1.51-1.36 (m, 1H).

### Examples #9 and 10

### (7aR,9R,11aS)-11a-Benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide; compound with (7aS,9S,11aR)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide (12, R²=Benzyl, R³=Methyl) and (7aR,9S,11aS)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide; compound with (7aS,9R,11aR)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide (20, R²=Benzyl, R³=Methyl)

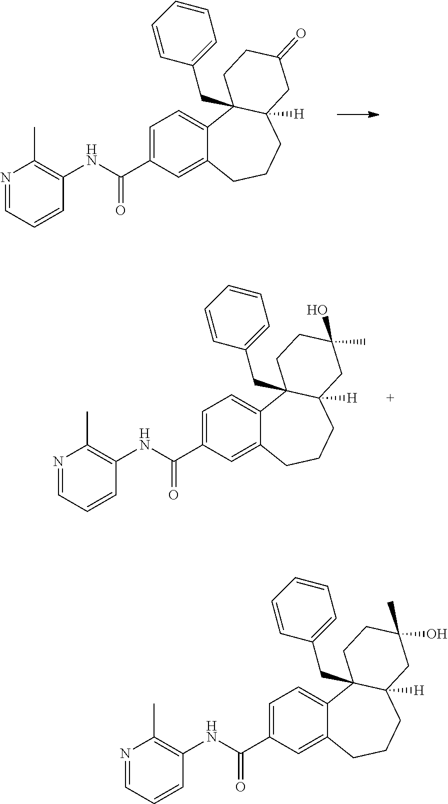

Methylmagnesium bromide (3M solution in Et₂O, 3.80 mL, 11.40 mmol) was cooled to about 0° C. under nitrogen and a solution of (7aR,11aS)-11a-benzyl-9-oxo-6,7,7a,8,9,10,11,11a-octahydro-5H-dibenzo[a,c]cycloheptene-3-carboxylic acid (2-methyl-pyridin-3-yl)-amide; compound with (7aS,11aR)-11a-benzyl-9-oxo-6,7,7a,8,9,10,11,11a-octahydro-5H-dibenzo[a,c]cycloheptene-3-carboxylic acid (2-methyl-pyridin-3-yl)-amide (18, R²=Benzyl) (0.500 g, 1.14 mmol) in THF (20 mL) was added dropwise over about 10 min. The mixture was stirred at about 0° C. for about 30 min then allowed to warm to rt. The reaction was quenched with 10% aqueous AcOH solution (30 mL) and the THF was removed under reduced pressure. The product was extracted with DCM (2×50 mL), dried over Na₂SO₄, filtered and concentrated under reduced pressure. The residue was purified on C18 (4 μm particle size 100×21 mm column) using a gradient (20 to 95%) MeCN in ammonium acetate buffer (50 mM). The minor peak fractions were collected and concentrated under reduced pressure to remove MeCN. The precipitate was collected by filtration and dried under reduced pressure to yield (7aR,9R,11aS)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide; compound with (7aS,9S,11aR)— 11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide (12, R²=Benzyl, R³=Methyl) (0.064 g, 12%) as a white solid. LC/MS, method 2, Rₜ=2.10 min, MS m/z 455 (M+H)⁺. ¹H NMR (400 MHz, DMSO-d₆) δ 9.93 (s, 1H), 8.31 (dd, J=4.7, 1.6 Hz, 1H), 7.79 (d, J=2.1 Hz, 1H), 7.71 (dd, J=8.0, 1.5 Hz, 1H), 7.56 (dd, J=8.4, 2.0 Hz, 1H), 7.25 (dd, J=7.9, 4.7 Hz, 1H), 7.06 (d, J=8.7 Hz, 1H), 7.05-6.99 (m, 3H), 6.52 (dd, J=6.5, 2.9 Hz, 2H), 4.36 (s, 1H), 3.49-3.39 (m, 2H), 3.05-2.95 (m, 2H), 2.41 (s, 3H), 2.07-1.82 (m, 3H), 1.85-1.38 (m, 8H), 1.11 (s, 3H). The major peak was collected, concentrated, filtered and dried under vacuum to yield (7aS,9R,11aR)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide; compound with (7aR,9S,11aS)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide (20, R²=Benzyl, R³=Methyl) (0.138 g, 27%) as a white solid. LC/MS, method 2, Rₜ=2.42 min; MS m/z: 455 (M+H)⁺. ¹H NMR (600 MHz, DMSO-d₆) δ 9.93 (s, 1H), 8.33 (dt, J=4.7, 2.5 Hz, 1H), 7.79 (t, J=4.0 Hz, 1H), 7.74 (dd, J=7.9, 1.4 Hz, 1H), 7.61-7.55 (m, 1H), 7.27 (dd, J=7.9, 4.7 Hz, 1H), 7.12 (t, J=8.0 Hz, 1H), 7.05-6.96 (m, 3H), 6.55 (dd, J=7.6, 1.7 Hz, 2H), 4.01 (s, 1H), 3.48-3.37 (m, 2H), 3.05-2.97 (m, 1H), 2.94-2.88 (m, 1H), 2.44 (s, 3H), 2.09-1.88 (m, 4H), 1.82-1.72 (m, 1H), 1.65-1.36 (m, 6H), 1.20 (s, 3H).

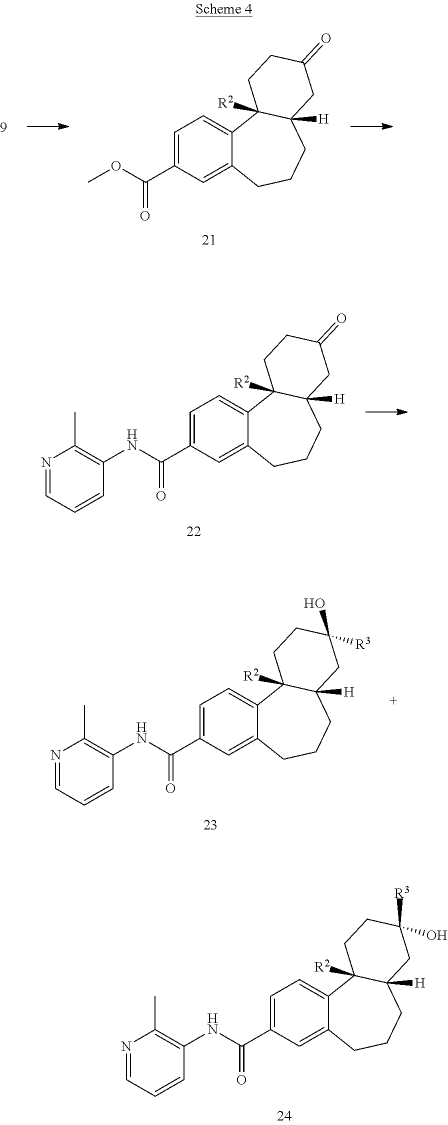

### Example #11

### (7aS,11aS)-11a-Benzyl-9-oxo-6,7,7a,8,9,10,11,11a-octahydro-5H-dibenzo[a,c]cycloheptene-3-carboxylic acid (2-methyl-pyridin-3-yl)-amide; compound with (7aR,11aR)-11a-benzyl-9-oxo-6,7,7a,8,9,10,11,11a-octahydro-5H-dibenzo[a,c]cycloheptene-3-carboxylic acid (2-methyl-pyridin-3-yl)-amide (22, R²=Benzyl)

### Step #1: (7aS,11aS)-11a-Benzyl-9-oxo-6,7,7a,8,9,10,11,11a-octahydro-5H-dibenzo[a,c]cycloheptene-3-carboxylic acid methyl ester; compound with (7aR,11aR)-11a-benzyl-9-oxo-6,7,7a,8,9,10,11,11a-octahydro-5H-dibenzo[a,c]cycloheptene-3-carboxylic acid methyl ester (21, R²=Benzyl)

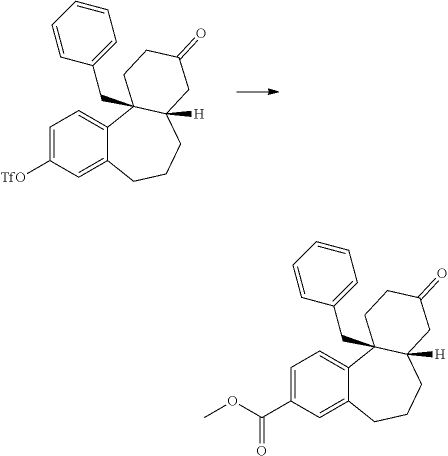

Trifluoro-methanesulfonic acid (7aS,11aS)-11a-benzyl-9-oxo-6,7,7a,8,9,10,11,11a-octahydro-5H-dibenzo[a,c]cyclohepten-3-yl ester; compound with trifluoro-methanesulfonic acid (7aR,11aR)-11a-benzyl-9-oxo-6,7,7a,8,9,10,11,11a-octahydro-5H-dibenzo[a,c]cyclohepten-3-yl ester (9, R²=Benzyl) (2.55 g, 5.64 mmol), Xantphos (0.326 g, 0.564 mmol) and Pd₂(dba)₃ (0.155 g, 0.169 mmol) were diluted with DMF (25 mL) and degassed by bubbling a stream of nitrogen. The reaction vessel was evacuated and an atmosphere of carbon monoxide was introduced via balloon. To the mixture was added MeOH (1.37 mL, 33.8 mmol) and TEA (1.57 mL, 11.3 mmol) and the reaction was heated at about 100° C. for about 4 h. The reaction was cooled and concentrated and the residue was purified on silica gel (80 g) using a gradient from 10 to 40% EtOAc in heptane. Product fractions were combined and concentrated to yield (7aS,11aS)-11a-benzyl-9-oxo-6,7,7a,8,9,10,11,11a-octahydro-5H-dibenzo[a,c]cycloheptene-3-carboxylic acid methyl ester; compound with (7aR,11aR)-11a-benzyl-9-oxo-6,7,7a,8,9,10,11,11a-octahydro-5H-dibenzo[a,c]cycloheptene-3-carboxylic acid methyl ester (21, R²=Benzyl) (1.30 g, 64%) as a white solid. LC/MS, method 1, Rₜ=0.92 min, MS m/z 380 (M+NH₄)⁺. ¹H NMR (400 MHz, DMSO-d₆) δ 7.81 (d, J=2.0 Hz, 1H), 7.55 (dd, J=8.3, 2.0 Hz, 1H), 7.10-6.99 (m, 3H), 6.91 (d, J=8.4 Hz, 1H), 6.53 (dd, J=7.7, 1.7 Hz, 2H), 3.82 (s, 3H), 3.69 (d, J=13.1 Hz, 1H), 3.33-3.23 (m, 1H), 3.06-2.96 (m, 1H), 2.61 (d, J=13.2 Hz, 1H), 2.45-2.16 (m, 5H), 2.12-1.75 (m, 4H), 1.71-1.64 (m, 1H), 1.59-1.49 (m, 1H).

### Step #2: (7aS,11aS)-11a-Benzyl-9-oxo-6,7,7a,8,9,10,11,11a-octahydro-5H-dibenzo[a,c]cycloheptene-3-carboxylic acid (2-methyl-pyridin-3-yl)-amide; compound with (7aR,11aR)-11a-benzyl-9-oxo-6,7,7a,8,9,10,11,11a-octahydro-5H-dibenzo[a,c]cycloheptene-3-carboxylic acid (2-methyl-pyridin-3-yl)-amide (22, R²=Benzyl)

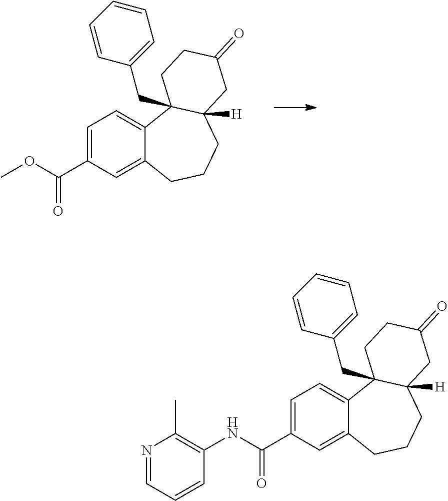

A solution of (7aS,11aS)-11a-benzyl-9-oxo-6,7,7a,8,9,10,11,11a-octahydro-5H-dibenzo[a,c]cycloheptene-3-carboxylic acid methyl ester; compound with (7aR,11aR)-11a-benzyl-9-oxo-6,7,7a,8,9,10,11,11a-octahydro-5H-dibenzo[a,c]cycloheptene-3-carboxylic acid methyl ester (21, R²=Benzyl) (1.30 g, 3.59 mmol) in 1,4-dioxane (20 mL) was treated with LiOH (0.452 g, 10.8 mmol) and water (5 mL). The reaction was warmed briefly to about 50° C. to obtain a homogeneous solution. The conversion to acid was followed to completion by LC/MS (method 1, Rₜ=0.74 min, MS m/z 347 (M−H)⁻. The mixture was acidified with 2N aqueous HCl (20 mL) and extracted with DCM (2×20 mL). The extracts were dried over Na₂SO₄, filtered and concentrated. The residue was dissolved in THF (25 mL) and DIEA (0.627 mL, 3.59 mmol) was added. The mixture was treated with TFFH (0.948 g, 3.59 mmol) at rt for about 5 min, and then with 2-methylpyridin-3-amine (0.776 g, 7.18 mmol) was added. The reaction was stirred for about 48 h at about 60° C. The reaction was cooled and concentrated. The residue was dissolved in DCM (60 mL) and washed with saturated aqueous NaHCO₃ (30 mL), dried over Na₂SO₄, filtered and concentrated. The crude product was purified on silica gel (80 g) using a gradient from 80% to 100% EtOAc in heptane. The product fractions were combined, concentrated and dried under reduced pressure to yield (7aS,11aS)-11a-benzyl-9-oxo-6,7,7a,8,9,10,11,11a-octahydro-5H-dibenzo[a,c]cycloheptene-3-carboxylic acid (2-methyl-pyridin-3-yl)-amide; compound with (7aR,11aR)-11a-benzyl-9-oxo-6,7,7a,8,9,10,11,11a-octahydro-5H-dibenzo[a,c]cycloheptene-3-carboxylic acid (2-methyl-pyridin-3-yl)-amide (22, R²=Benzyl) (1.01 g, 64%) as an off-white solid. LC/MS, method 1, Rₜ=0.77 min, MS m/z 439 (M+H)⁺. ¹H NMR (400 MHz, DMSO-d₆) δ 10.00 (s, 1H), 8.34 (dd, J=4.7, 1.6 Hz, 1H), 7.88 (d, J=2.1 Hz, 1H), 7.74 (dd, J=7.9, 1.6 Hz, 1H), 7.64 (dd, J=8.2, 2.1 Hz, 1H), 7.27 (dd, J=7.9, 4.7 Hz, 1H), 7.15-7.01 (m, 3H), 6.96 (d, J=8.3 Hz, 1H), 6.64-6.58 (m, 2H), 3.73 (d, J=13.0 Hz, 1H), 3.40-3.09 (m, 1H). 3.09-2.99 (m, 1H), 2.66 (d, J=13.1 Hz, 1H), 2.44 (s, 3H), 2.42-2.23 (m, 5H), 2.18-2.05 (m, 1H), 2.03-1.80 (m, 3H), 1.76-1.51 (m, 2H).

### Examples #12 and 13

### (7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide (23, R²=Benzyl, R₃=Ethyl) and (7aS,9S,11aS)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide (24, R²=Benzyl, R³=Ethyl)

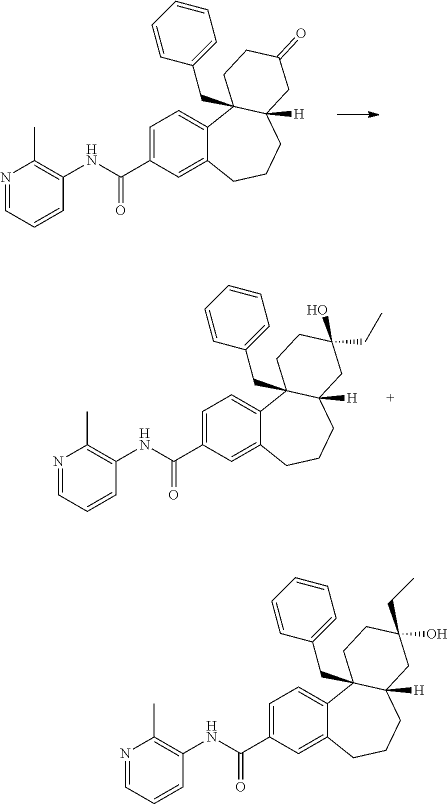

Ethylmagnesium bromide (3M solution in Et₂O, 1.10 mL, 3.31 mmol) was cooled to about 0° C. and a slurry of (7aS,11aS)-11a-benzyl-9-oxo-6,7,7a,8,9,10,11,11a-octahydro-5H-dibenzo[a,c]cycloheptene-3-carboxylic acid (2-methyl-pyridin-3-yl)-amide; compound with (7aR,11aR)-11a-benzyl-9-oxo-6,7,7a,8,9,10,11,11a-octahydro-5H-dibenzo[a,c]cycloheptene-3-carboxylic acid (2-methyl-pyridin-3-yl)-amide (22, R²=Benzyl) (145 mg, 0.331 mmol) in THF (6 mL) was added dropwise. The reaction was stirred at about 0° C. for about 30 min then quenched by addition of 10% aqueous AcOH (10 mL). The reaction was concentrated under reduced pressure then extracted with EtOAc (2×25 mL). The combined organics were dried over Na₂SO₄, filtered and concentrated under reduced pressure. The residue was purified on silica gel (40 g) using a gradient from 80 to 100% EtOAc in heptane. The two products were isolated separately. Each was concentrated under reduced pressure, then precipitated from MeCN and water. The products were collected by filtration and dried under vacuum to yield (7aS,9R,11aS)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide (23, R²=Benzyl, R³=Ethyl) (58 mg, 37%) as a white solid, LC/MS, method 2, Rₜ=2.33 min, MS m/z 469 (M+H)⁺. ¹H NMR (400 MHz, DMSO-d₆) δ 9.92 (s, 1H), 8.31 (dd, J=4.7, 1.5 Hz, 1H), 7.79 (d, J=1.9 Hz, 1H), 7.72 (dd, J=8.0, 1.4 Hz, 1H), 7.53 (dd, J=8.2, 1.9 Hz, 1H), 7.25 (dd, J=7.9, 4.8 Hz, 1H), 7.14-7.00 (m, 3H), 6.80 (d, J=8.5 Hz, 1H), 6.57 (dd, J=6.4, 2.9 Hz, 2H), 3.87 (s, 1H), 3.56 (d, J=12.9 Hz, 1H), 3.33-3.23 (m, 1H), 3.07-2.98 (m, 1H), 2.64-2.56 (d, 1H), 2.49-2.40 (m, 5H), 1.94-1.70 (m, 3H), 1.68-1.23 (m, 4H), 1.20-1.02 (m, 4H), 0.71 (t, J=7.4, 3H) and (7aS,9S,11aS)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide (24, R²=Benzyl, R³=Ethyl) (22 mg, 14%) as a white solid, LC/MS, method 2, Rₜ=2.55 min, MS m/z 469 (M+H)⁺. ¹H NMR (400 MHz, DMSO-d₆) δ 9.95 (s, 1H), 8.35-8.31 (m, 1H), 7.84-7.80 (m, 1H), 7.76-7.71 (m, 1H), 7.62-7.56 (m, 1H), 7.30-7.24 (m, 1H), 7.10-7.01 (m, 3H), 6.90-6.77 (m, 1H), 6.62-6.53 (m, 2H), 3.88 (s, 1H), 3.58-3.51 (m, 1H), 3.28-3.16 (m, 1H), 3.07-2.93 (m, 1H), 2.59 (d, J=13.0 Hz, 1H), 2.47-2.33 (m, 4H), 2.06-1.73 (m, 3H), 1.72-1.39 (m, 7H), 1.32-1.27 (m, 2H), 0.84-0.77 (m, 3H).

### Examples #14 and #15

### Chiral purification of (7aS,9R,11aS)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide (23, R²=Benzyl, R³=Ethyl)

The enantiomers were separated by chiral preparative chromatography (Isocratic 30% A). Mobile phase A was EtOH (200 proof), mobile phase B was HPLC grade heptane with 0.12% DEA added. The column used for the chromatography was a Daicel IA, 20×250 mm column (5 μm particles) to provide first (7aS,9R,11aS)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide (23 (7aS,9R,11aS), R²=Benzyl, R³=Ethyl) and second (7aR,9S,11aR)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide (23 (7aR,9S,11aR), R²=Benzyl, R³=Ethyl). NMR and LC/MS data for single isomers was essentially identical to the racemic mixture.

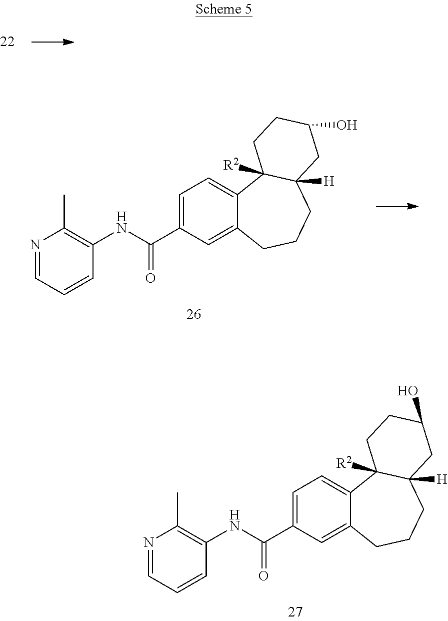

### Example #16

### (7aS,9S,11aS)-11a-Benzyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-11a-benzyl-9-hydroxy-6,7,7a,8,9,10,11,11a-octahydro-5H-dibenzo[a,c]cycloheptene-3-carboxylic acid (2-methyl-pyridin-3-yl)-amide (26, R²=Benzyl)

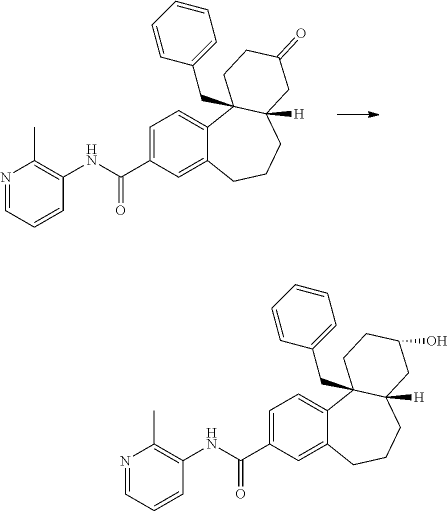

A suspension of (7aS,11aS)-11a-benzyl-9-oxo-6,7,7a,8,9,10,11,11a-octahydro-5H-dibenzo[a,c]cycloheptene-3-carboxylic acid (2-methyl-pyridin-3-yl)-amide; compound with (7aR,11aR)-11a-benzyl-9-oxo-6,7,7a,8,9,10,11,11a-octahydro-5H-dibenzo[a,c]cycloheptene-3-carboxylic acid (2-methyl-pyridin-3-yl)-amide (22, R²=Benzyl) (100 mg, 0.228 mmol) in EtOH (2 mL) was treated with sodium borohydride (10.4 mg, 0.274 mmol) and the reaction was stirred at rt for about 4 h. The reaction was concentrated under reduced pressure and the residue was triturated with water (2 mL), filtered and purified on silica gel (12 g) using 80-100% EtOAc in heptane. Product fractions were combined and concentrated under reduced pressure. The residue was dissolved in MeCN (5 mL) and the product precipitated. The product was filtered off and dried under vacuum to yield (7aS,9S,11aS)-11a-benzyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-11a-benzyl-9-hydroxy-6,7,7a,8,9,10,11,11a-octahydro-5H-dibenzo[a,c]cycloheptene-3-carboxylic acid (2-methyl-pyridin-3-yl)-amide (26, R²=Benzyl) (56 mg, 56%) as a white solid, LC/MS, method 1, Rₜ=2.55 min, MS m/z 441 (M+H)⁺. ¹H NMR (400 MHz, DMSO-d₆) δ 9.95 (s, 1H), 8.33 (dd, J=4.7, 1.6 Hz, 1H), 7.82 (d, J=2.1 Hz, 1H), 7.74 (dd, J=7.9, 1.6 Hz, 1H), 7.59 (dd, J=8.2, 2.1 Hz, 1H), 7.27 (dd, J=7.9, 4.7 Hz, 1H), 7.12-7.02 (m, 3H), 6.82 (d, J=8.4 Hz, 1H), 6.59 (d, J=1.9 Hz, 2H), 4.40 (d, J=4.7 Hz, 1H), 3.62-3.52 (m, 2H), 3.33-3.22 (m, 1H), 3.08-2.98 (m, 1H), 2.54 (d, J=13.0 Hz, 1H), 2.47-2.37 (m, 4H), 2.13-2.05 (m, 1H), 1.93-1.67 (m, 4H), 1.63-1.22 (m, 4H), 1.14-1.01 (m, 1H).

### Example #17

### (7aS,9R,11aS)-11a-Benzyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-hydroxy-6,7,7a,8,9,10,11,11a-octahydro-5H-dibenzo[a,c]cycloheptene-3-carboxylic acid (2-methyl-pyridin-3-yl)-amide (27, R²=Benzyl)

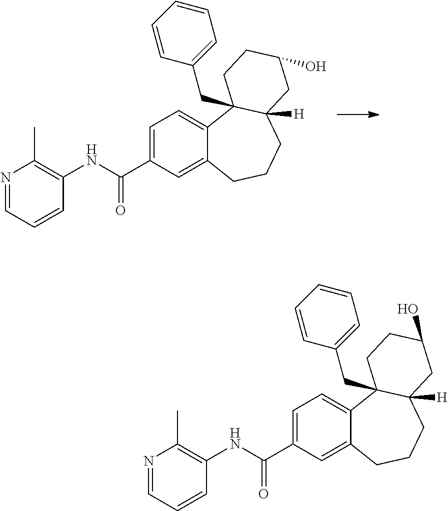

A solution of crude (7aS,9S,11aS)-11a-benzyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-11a-benzyl-9-hydroxy-6,7,7a,8,9,10,11,11a-octahydro-5H-dibenzo[a,c]cycloheptene-3-carboxylic acid (2-methyl-pyridin-3-yl)-amide (26, R²=Benzyl) (100 mg, 0.228 mmol) in THF (0.50 mL) was treated with triphenylphosphine (71.8 mg, 0.274 mmol). A solution of DBAD (0.063 g, 0.27 mmol) and 4-nitro-benzoic acid (0.028 mL, 0.274 mmol) in THF (0.50 mL) was added dropwise. The mixture was stirred at rt for about 18 h. The intermediate ester was treated with 2N aqueous NaOH (0.50 mL) and the mixture was stirred at rt for about 2 h. The mixture was concentrated to remove THF and product was extracted into EtOAc (2×10 mL). The residue was purified on silica gel (12 g) using a gradient from 80 to 100% EtOAc in heptane. The product fractions were combined and concentrated. The product was precipitated from MeCN with water, then collected by filtration and dried under reduced pressure to yield (7aS,9R,11aS)-11a-benzyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-hydroxy-6,7,7a,8,9,10,11,11a-octahydro-5H-dibenzo[a,c]cycloheptene-3-carboxylic acid (2-methyl-pyridin-3-yl)-amide (27, R²=Benzyl) (18 mg, 18%) as a white solid. LC/MS, method 2, Rₜ=2.13 min, MS m/z 441 (M+H)⁺. ¹H NMR (400 MHz, DMSO-d₆) δ 9.93 (s, 1H), 8.33 (d, J=4.5 Hz, 1H), 7.83-7.70 (m, 2H), 7.65-7.50 (m, 2H), 7.27 (dd, J=7.8, 4.8 Hz, 1H), 7.12-7.02 (m, 3H), 6.66-6.52 (m, 2H), 4.39 (s, 1H), 3.81-3.69 (m, 1H), 3.55 (d, J=12.9 Hz, 1H), 3.21-3.31 (m, 1H), 3.10-2.87 (m, 1H), 2.64 (d, J=13.2 Hz, 1H), 2.47-2.37 (m, 4H), 1.92-1.71 (m, 3H), 1.66-1.48 (m, 5H), 1.43-1.19 (m, 2H).

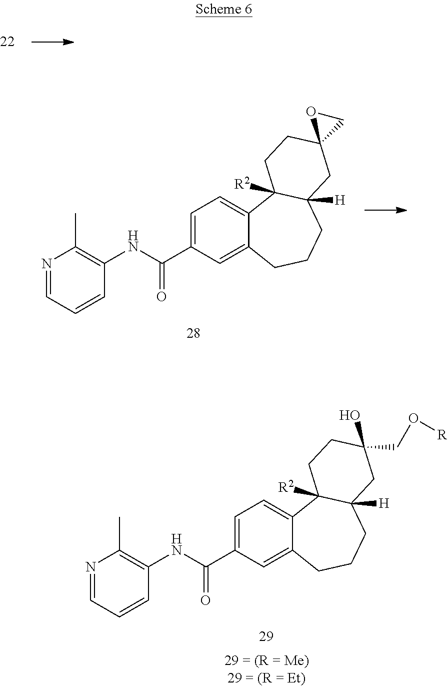

### Preparation #2: (+/−) Compound 28 (R²=Benzyl)

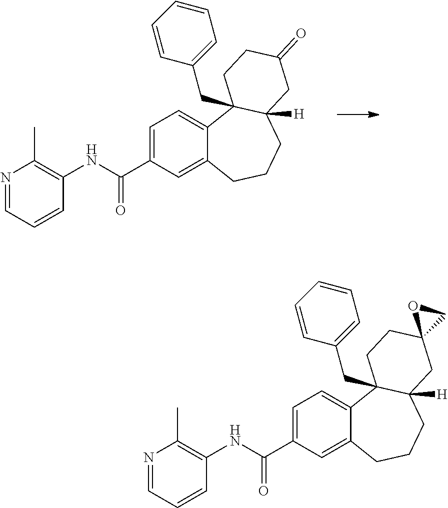

A 60% dispersion of NaH (0.073 g, 1.82 mmol) in mineral oil was dissolved in dry DMSO-d₆ (5.0 mL) and the mixture was heated at about 60° C. for about 30 min. The mixture was allowed to cool to rt, then THF (5 mL) was added and the reaction mixture was cooled to about −20° C. To the mixture was added trimethylsulfoxonium iodide (0.410 g, 1.82 mmol) and a suspension of (7aS,11aS)-11a-benzyl-9-oxo-6,7,7a,8,9,10,11,11a-octahydro-5H-dibenzo[a,c]cycloheptene-3-carboxylic acid (2-methyl-pyridin-3-yl)-amide; compound with (7aR,11aR)-11a-benzyl-9-oxo-6,7,7a,8,9,10,11,11a-octahydro-5H-dibenzo[a,c]cycloheptene-3-carboxylic acid (2-methyl-pyridin-3-yl)-amide (22, R²=Benzyl) (0.400 g, 0.912 mmol) in THF (2 mL) and the reaction was stirred for about 18 h at rt. A saturated aqueous solution of NaHCO₃ (50 mL) was added, and the mixture was extracted with EtOAc (2×25 mL). The organic extracts were combined and washed with saturated aqueous NaCl (25 mL), dried over Na₂SO₄, filtered and concentrated under reduced pressure. The residue was purified on silica gel (12 g) using a gradient from 40-100% EtOAc in heptane. Product fractions were combined, concentrated under reduced pressure and dried under vacuum to yield (+/−) Compound 28 (R²=Benzyl) (0.371 g, 81%) as a white solid, LC/MS, method 2, Rₜ=2.46 min, MS m/z 453 (M+H)⁺. ¹H NMR (400 MHz, DMSO-d₆) δ 10.00 (s, 1H), 8.34 (dd, J=4.7, 1.6 Hz, 1H), 7.88 (d, J=2.1 Hz, 1H), 7.74 (dd, J=7.9, 1.6 Hz, 1H), 7.64 (dd, J=8.2, 2.1 Hz, 1H), 7.27 (dd, J=7.9, 4.7 Hz, 1H), 7.15-7.01 (m, 3H), 6.96 (d, J=8.3 Hz, 1H), 6.67-6.60 (m, 2H), 3.73 (d, J=13.0 Hz, 1H), 3.34-3.21 (m, 1H), 3.09-2.99 (m, 1H), 2.66 (d, J=13.1 Hz, 1H), 2.53 (s, 2H), 2.49-2.40 (m, 4H), 2.35-2.25 (m, 1H), 2.19-2.10 (m, 1H), 2.10-1.99 (m, 1H), 1.87-1.63 (m, 4H), 1.63-1.47 (m, 1H), 1.20-1.07 (m, 1H), 0.94-0.77 (m, 1H).

### Example #18

### (7aS,9R,11aS)-11a-Benzyl-9-hydroxy-9-methoxymethyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-hydroxy-9-methoxymethyl-6,7,7a,8,9,10,11,11a-octahydro-5H-dibenzo[a,c]cycloheptene-3-carboxylic acid (2-methyl-pyridin-3-yl)-amide (29, R=Methyl, R²=Benzyl)

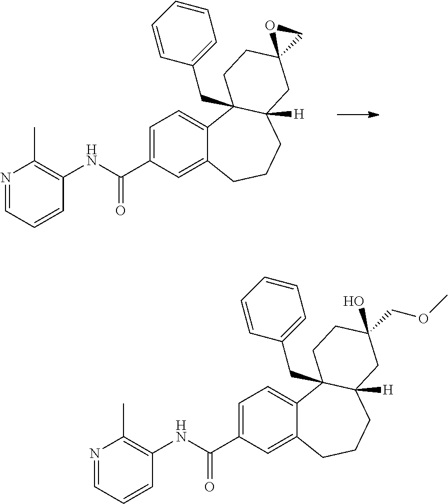

A solution of (+/−) Compound 28 (R²=Benzyl) (0.108 g, 0.239 mmol) in MeOH (5.0 mL) was treated with sodium methoxide (0.027 g, 0.50 mmol) and the reaction was stirred at about 60° C. for about 18 h. The reaction was cooled and concentrated under reduced pressure. The residue was dissolved in EtOAc (30 mL) and washed with water (1×25 mL). The organic layer was dried over Na₂SO₄, filtered and concentrated. The residue was purified on silica gel (4 g) using a gradient from 70-100% EtOAc in heptane. Product fractions were combined, concentrated and dried under vacuum to yield (7aS,9R,11aS)-11a-benzyl-9-hydroxy-9-methoxymethyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-hydroxy-9-methoxymethyl-6,7,7a,8,9,10,11,11a-octahydro-5H-dibenzo[a,c]cycloheptene-3-carboxylic acid (2-methyl-pyridin-3-yl)-amide (22, R=Methyl, R²=Benzyl) (49 mg, 42%) as a white foam, LC/MS, method 2, Rₜ=2.21 min, MS m/z 485 (M+H)⁺. ¹H NMR (400 MHz, DMSO-d₆) δ 9.95 (s, 1H), 8.33 (dd, J=4.7, 1.6 Hz, 1H), 7.81 (d, J=1.9 Hz, 1H), 7.77-7.68 (m, 1H), 7.56 (dd, J=8.2, 2.1 Hz, 1H), 7.27 (dd, J=7.8, 4.8 Hz, 1H), 7.09-7.03 (m, 3H), 6.82 (d, J=8.5 Hz, 1H), 6.60-6.54 (m, 2H), 4.22 (s, 1H), 3.57 (d, J=12.9 Hz, 1H), 3.33-3.23 (m, 1H), 3.12 (s, 3H), 3.06-3.00 (m, 1H), 2.94 (s, 2H), 2.60 (d, J=13.1 Hz, 1H), 2.50-2.40 (m, 5H), 1.96-1.70 (m, 3H), 1.71-1.44 (m, 3H), 1.45-1.20 (m, 2H), 1.12-1.06 (m, 1H).

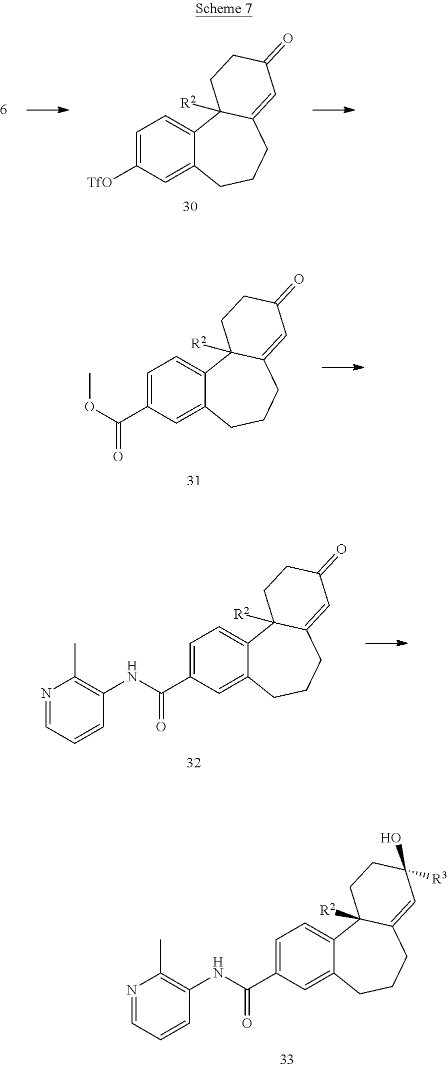

### Example #19

### (9R,11aS)-11a-Benzyl-9-hydroxy-9-methyl-6,7,9,10,11,11a-hexahydro-5H-dibenzo[a,c]cycloheptene-3-carboxylic acid (2-methyl-pyridin-3-yl)-amide; compound with (9S,11aR)-11a-benzyl-9-hydroxy-9-methyl-6,7,9,10,11,11a-hexahydro-5H-dibenzo[a,c]cycloheptene-3-carboxylic acid (2-methyl-pyridin-3-yl)-amide (33, R²=Benzyl, R³=Methyl)

### Step #1: Trifluoro-methanesulfonic acid 11a-benzyl-9-oxo-6,7,9,10,11,11a-hexahydro-5H-dibenzo[a,c]cyclohepten-3-yl ester (30)

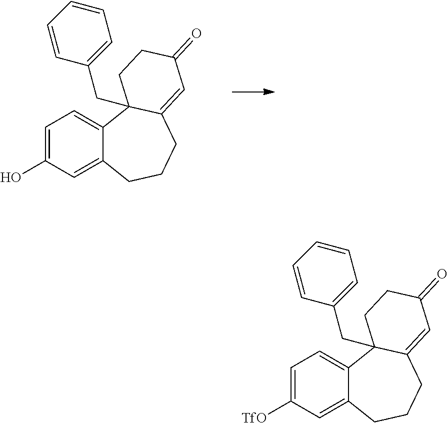

A slurry of 11b-benzyl-9-hydroxy-1,2,5,6,7,11b-hexahydro-dibenzo[a,c]cyclohepten-3-one (6, R²=Benzyl) (0.239 g, 0.751 mmol) in DCM (5 mL) was treated with N-phenylbis(trifluoromethanesulfonimide) (0.268 g, 0.75 mmol) and DIEA (0.262 mL, 1.50 mmol) and the reaction was stirred at rt for about 18 h. Silica gel (5.0 g) was added and solvent was removed under vacuum. The residue was purified on silica gel (25 g) using a gradient from 10-30% EtOAc in heptane. The product fractions were combined, concentrated and dried under vacuum to yield trifluoro-methanesulfonic acid 11a-benzyl-9-oxo-6,7,9,10,11,11a-hexahydro-5H-dibenzo[a,c]cyclohepten-3-yl ester (30, R²=Benzyl) (206 mg, 61%) as an oil, LC/MS, method 1, Rₜ=0.97 min, MS m/z 451 (M+H)⁺. ¹H NMR (400 MHz, DMSO-d₆) δ 7.91 (d, J=8.7 Hz, 1H), 7.42-7.35 (m, 1H), 7.26 (d, J=2.5 Hz, 1H), 7.17-7.10 (m, 3H), 7.05-6.99 (m, 2H), 5.92 (s, 1H), 3.64-3.52 (m, 1H), 3.49-3.40 (m, 1H), 2.96-2.83 (m, 1H), 2.75-2.61 (m, 2H), 2.35-2.21 (m, 2H), 2.14-2.01 (m, 2H), 1.80-1.62 (m, 2H), 1.56-1.42 (m, 1H)

### Step #2: 11a-Benzyl-9-oxo-6,7,9,10,11,11a-hexahydro-5H-dibenzo[a,c]cycloheptene-3-carboxylic acid methyl ester (31, R²=Benzyl)

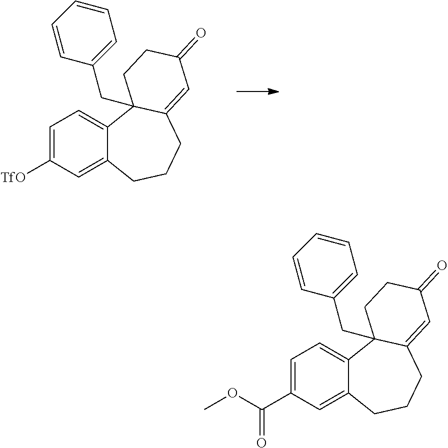

A solution of trifluoro-methanesulfonic acid 11a-benzyl-9-oxo-6,7,9,10,11,11a-hexahydro-5H-dibenzo[a,c]cyclohepten-3-yl ester (30, R²=Benzyl) (0.202 g, 0.448 mmol) in DMF (1.50 mL) was treated with Xantphos (0.026 g, 0.045 mmol) and tris(benzylideneacetone)dipalladium(0) (0.012 g, 0.013 mmol) and the mixture was degassed with a stream of nitrogen, then evacuated. An atmosphere of carbon monoxide was introduced via balloon and then TEA (0.125 mL, 0.897 mmol) and MeOH (0.109 mL, 2.69 mmol) were added. The mixture was heated at about 100° C. for about 18 h, then cooled and concentrated under reduced pressure. The residue was purified on silica gel (25 g) using a gradient from 10 to 40% EtOAc in heptane. The product fractions were combined, concentrated and dried under reduced pressure to yield 11a-benzyl-9-oxo-6,7,9,10,11,11a-hexahydro-5H-dibenzo[a,c]cycloheptene-3-carboxylic acid methyl ester (31, R²=Benzyl) (0.105 g, 65%) as an amorphous solid, LC/MS, method 1, Rₜ=0.81 min, MS m/z 361 (M+H)⁺. ¹H NMR (400 MHz, DMSO-d₆) δ 7.94 (d, J=8.3 Hz, 1H), 7.86 (dd, J=8.3, 1.9 Hz, 1H), 7.68 (d, J=1.9 Hz, 1H), 7.14-7.07 (m, 3H), 7.06-6.99 (m, 2H), 5.92 (s, 1H), 3.82 (s, 3H), 3.63 (d, J=13.6 Hz, 1H), 3.39 (d, J=13.6, 1H), 2.95-2.82 (m, 1H), 2.77-2.62 (m, 2H), 2.33-2.20 (m, 2H), 2.12-2.01 (m, 2H), 1.78-1.64 (m, 2H), 1.47-1.34 (m, 1H).

### Step #3: 11a-Benzyl-9-oxo-6,7,9,10,11,11a-hexahydro-5H-dibenzo[a,c]cycloheptene-3-carboxylic acid (2-methyl-pyridin-3-yl)-amide (32, R²=Benzyl)

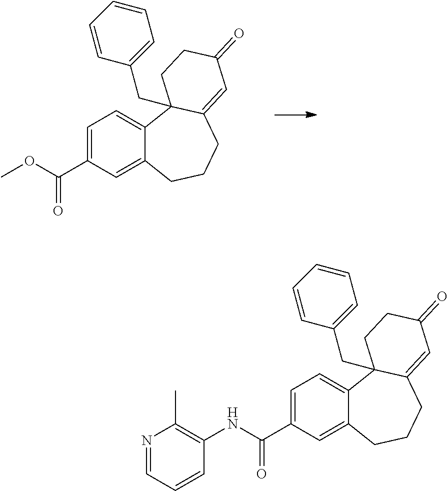

To a solution of 11a-benzyl-9-oxo-6,7,9,10,11,11a-hexahydro-5H-dibenzo[a,c]cycloheptene-3-carboxylic acid methyl ester (31, R²=Benzyl) (102 mg, 0.283 mmol) in 1,4-dioxane (2.0 mL) was added LiOH monohydrate (0.059 g, 1.41 mmol) in water (0.50 mL) and the mixture was stirred at about 50° C. for about 1 h. The reaction was concentrated, 2N aqueous HCl was added to adjust the pH to about 1 and the intermediate was extracted with DCM (2×5 mL). The combined extracts were dried over Na₂SO₄, filtered and concentrated under reduced pressure. The residue was dissolved in THF (3 mL), and 2-methyl-pyridin-3-ylamine (61.2 mg, 0.566 mmol), DIEA (0.049 mL, 0.28 mmol) and TFFH (74.7 mg, 0.283 mmol) were added. The mixture was stirred at rt for about 15 min then heated at about 60° C. for about 18 h. The reaction was cooled and concentrated under reduced pressure. The residue was dissolved in DCM (5.0 mL) and washed with saturated aqueous NaHCO₃ (2×5 mL). The organic layer was dried over Na₂SO₄, filtered and concentrated under reduced pressure. The residue was purified on silica gel (12 g) using a gradient from 80-100% EtOAc in heptane. The product fractions were combined, concentrated and dried under vacuum to yield 11a-benzyl-9-oxo-6,7,9,10,11,11a-hexahydro-5H-dibenzo[a,c]cycloheptene-3-carboxylic acid (2-methyl-pyridin-3-yl)-amide (32, R²=Benzyl), LC/MS, method 1, Rₜ=0.67 min, MS m/z 437 (M+H)⁺, which was used in the next step without further purification.

### Step #4: (9R,11aS)-11a-Benzyl-9-hydroxy-9-methyl-6,7,9,10,11,11a-hexahydro-5H-dibenzo[a,c]cycloheptene-3-carboxylic acid (2-methyl-pyridin-3-yl)-amide; compound with (9S,11aR)-11a-benzyl-9-hydroxy-9-methyl-6,7,9,10,11,11a-hexahydro-5H-dibenzo[a,c]cycloheptene-3-carboxylic acid (2-methyl-pyridin-3-yl)-amide (33, R²=Benzyl, R³=Methyl)

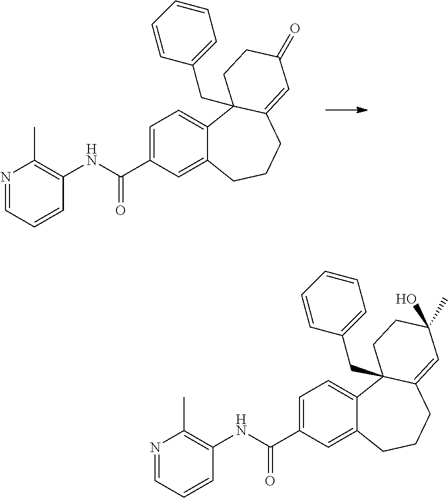

A solution of 3M methylmagnesium bromide (0.916 mL, 2.75 mmol) in Et₂O was cooled to about 0° C. and a slurry of 11a-benzyl-9-oxo-6,7,9,10,11,11a-hexahydro-5H-dibenzo[a,c]cycloheptene-3-carboxylic acid (2-methyl-pyridin-3-yl)-amide (32, R²=Benzyl) (120 mg, 0.275 mmol) in THF (5.0 mL) was added dropwise. The reaction was stirred at about 0° C. for about 30 min then quenched by addition of 10% aqueous AcOH (15 mL). The reaction was concentrated under vacuum then extracted with DCM (2×15 mL). The organic extracts were dried over Na₂SO₄, filtered and concentrated. The residue was purified on silica gel (40 g) using EtOAc as eluent. Product fractions were combined, concentrated and dried under vacuum to yield (9R,11aS)-11a-benzyl-9-hydroxy-9-methyl-6,7,9,10,11,11a-hexahydro-5H-dibenzo[a,c]cycloheptene-3-carboxylic acid (2-methyl-pyridin-3-yl)-amide; compound with (9S,11aR)-11a-benzyl-9-hydroxy-9-methyl-6,7,9,10,11,11a-hexahydro-5H-dibenzo[a,c]cycloheptene-3-carboxylic acid (2-methyl-pyridin-3-yl)-amide (33, R²=Benzyl, R³=Methyl) (10 mg, 8%), LC/MS, method 3, Rₜ=2.11 min, MS m/z 453 (M+H)⁺. ¹H NMR (400 MHz, DMSO-d₆) δ 9.93 (s, 1H), 8.31 (dd, J=4.8, 1.6 Hz, 1H), 7.75-7.68 (m, 3H), 7.35 (d, J=8.8 Hz, 1H), 7.25 (dd, J=8.0, 4.7 Hz, 1H), 7.14-7.09 (m, 3H), 6.95-6.90 (m, 2H), 5.37 (s, 1H), 4.45 (s, 1H), 3.43 (d, J=13.2 Hz, 1H), 3.12 (d, J=13.2 Hz, 1H), 3.07-2.84 (m, 2H), 2.42 (s, 3H), 2.08-2.00 (m, 2H), 1.94-1.74 (m, 4H), 1.65-1.54 (m, 1H), 1.54-1.43 (m, 1H), 1.06 (s, 3H).

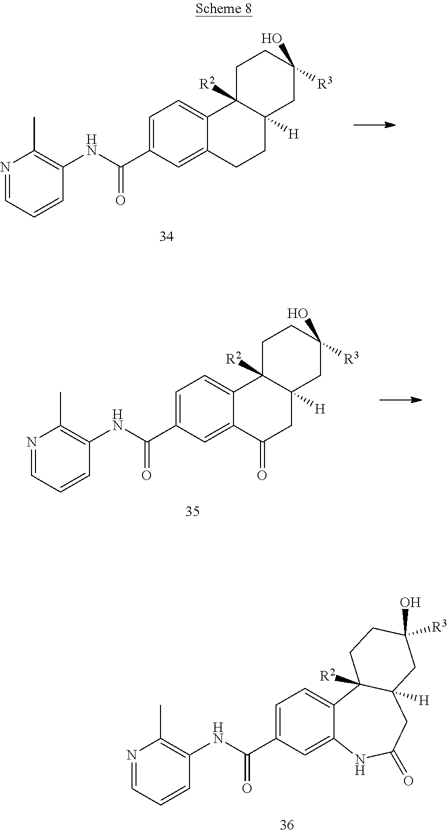

### Example 20

### (7aS,9R,11aS)-11a-Benzyl-9-hydroxy-N-(2-methylpyridin-3-yl)-6-oxo-9-(trifluoromethyl)-6,7,7a,8,9,10,11,11a-octahydro-5H-dibenzo[b,d]azepine-3-carboxamide (36, R²=Benzyl, R³=Trifluoromethyl)

### Step #1: (4bS,7R,8aS)-4b-Benzyl-7-hydroxy-N-(2-methylpyridin-3-yl)-10-oxo-7-(trifluoromethyl)-4b,5,6,7,8,8a,9,10-octahydrophenanthrene-2-carboxamide (35, R²=Benzyl, R³=Trifluoromethyl)

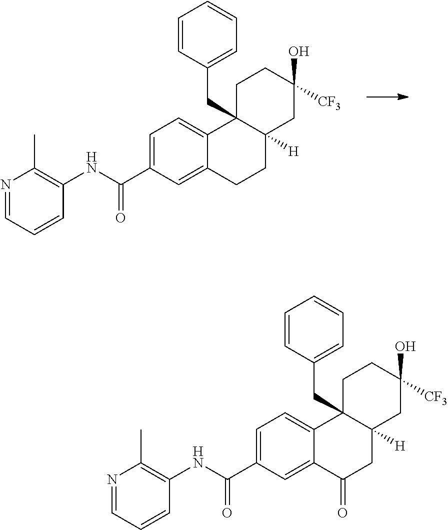

A 100 mL round bottom flask equipped with a nitrogen inlet adapter was charged with (4bS,7R,8aR)-4b-benzyl-7-hydroxy-N-(2-methylpyridin-3-yl)-7-(trifluoromethyl)-4b,5,6,7,8,8a,9,10-octahydrophenanthrene-2-carboxamide (34, R²=Benzyl, R³=Trifluoromethyl) (0.343 g, 0.694 mmol; prepared as described in WO 2008093236 A1) in DCM (7 mL) to give a tan solution. The sample was cooled at about −78° C. and treated with ozone gas at about 4 psi over about 5 min and then the vessel was subsequently treated with about 4 psi of ozone for about 5 min at periodic intervals of approximately 1 h to 18 h for an additional period of time of approximately 30 h. The sample was treated with a molar excess of PS-PPh₃ for about 4 h. EtOAc was added and the suspension was filtered through a pad of Celite®. The filtrate was purified via silica gel chromatography eluting with a gradient of 0-10% MeOH in DCM. The fractions containing product were combined and concentrated to afford (4bS,7R,8aS)-4b-benzyl-7-hydroxy-N-(2-methylpyridin-3-yl)-10-oxo-7-(trifluoromethyl)-4b,5,6,7,8,8a,9,10-octahydrophenanthrene-2-carboxamide (35, R²=Benzyl, R³=Trifluoromethyl) (0.215 g, 61%) as a solid. LC/MS, method 2, Rₜ=2.09 min, MS m/z 509 (M+H)⁺. ¹H NMR (400 MHz, DMSO-d₆) δ 10.23 (s, 1H), 8.56 (d, J=2.1 Hz, 1H), 8.35 (dd, J=4.8, 1.6 Hz, 1H), 7.91 (dd, J=8.3, 2.1 Hz, 1H), 7.74 (d, J=6.5 Hz, 1H), 7.28 (dd, J=8.0, 4.8 Hz, 1H), 7.18-7.07 (m, 3H), 6.69 (d, J=8.3 Hz, 1H), 6.55-6.50 (m, 2H), 6.11 (s, 1H), 5.75 (s, 1H), 3.33-3.26 (m, 1H), 2.91-2.81 (m, 1H), 2.76-2.61 (m, 2H), 2.42 (s, 3H), 2.30-2.06 (m, 4H), 2.05-1.89 (m, 1H), 1.40-1.50 (m, 1H).

### Step #2: (7aS,9R,11aS)-11a-Benzyl-9-hydroxy-N-(2-methylpyridin-3-yl)-6-oxo-9-(trifluoromethyl)-6,7,7a,8,9,10,11,11a-octahydro-5H-dibenzo[b,d]azepine-3-carboxamide (36, R²=Benzyl, R³=Trifluoromethyl)

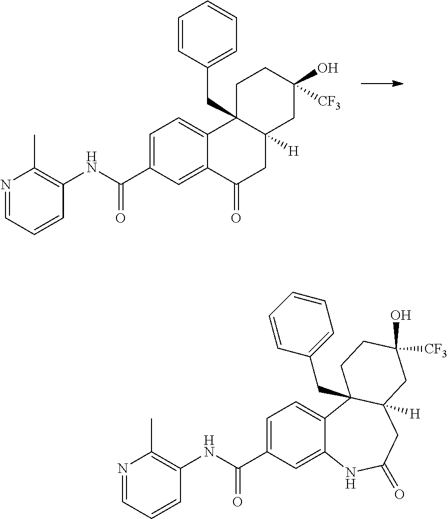

A 10 mL reaction vial equipped with a nitrogen inlet adapter was charged with (4bS,7R,8aS)-4b-benzyl-7-hydroxy-N-(2-methylpyridin-3-yl)-10-oxo-7-(trifluoromethyl)-4b,5,6,7,8,8a,9,10-octahydrophenanthrene-2-carboxamide (35, R²=Benzyl, R³=Trifluoromethyl) (0.070 g, 0.14 mmol) and MsOH (0.089 mL, 1.4 mmol) in DCM (1.4 mL) to give a colorless suspension. Sodium azide (0.018 g, 0.28 mmol) was added in one portion. The resulting solution was allowed to stir at rt for about 5 h. The reaction mixture was partitioned between DCM (20 mL) and H₂O (20 mL). The organic phase was washed with saturated aqueous NaHCO₃ (2×20 mL), H₂O (10 mL), and saturated aqueous NaCl (10 mL). The organic phase was dried over MgSO₄, filtered and concentrated under reduced pressure to give a solid. The sample was purified via silica gel chromatography eluting with 5-10% MeOH in DCM. The fractions containing product were combined and concentrated under reduced pressure to afford (7aS,9R,11aS)-11a-benzyl-9-hydroxy-N-(2-methylpyridin-3-yl)-6-oxo-9-(trifluoromethyl)-6,7,7a,8,9,10,11,11a-octahydro-5H-dibenzo[b,d]azepine-3-carboxamide (36, R²=Benzyl, R³=Trifluoromethyl) (0.033 g, 44%) as a solid. LC/MS, method 2, Rₜ=1.72 min, MS m/z 524 (M+H)⁺. ¹H NMR (400 MHz, DMSO-d₆) δ 10.12 (s, 1H), 9.99 (s, 1H), 8.34 (dd, J=4.7, 1.6 Hz, 1H), 7.79-7.73 (m, 2H), 7.51 (dd, J=8.0, 2.0 Hz, 1H), 7.30-7.24 (m, 1H), 7.11-7.07 (m, 3H), 7.03 (d, J=8.3 Hz, 1H), 6.69-6.63 (m, 2H), 6.04-5.99 (m, 1H), 2.92 (d, J=13.2 Hz, 1H), 2.87-2.77 (m, 1H), 2.68-2.64 (m, 1H), 2.45 (s, 3H), 2.42-2.30 (m, 1H), 2.19-2.02 (m, 1H), 1.93-1.71 (m, 6H).

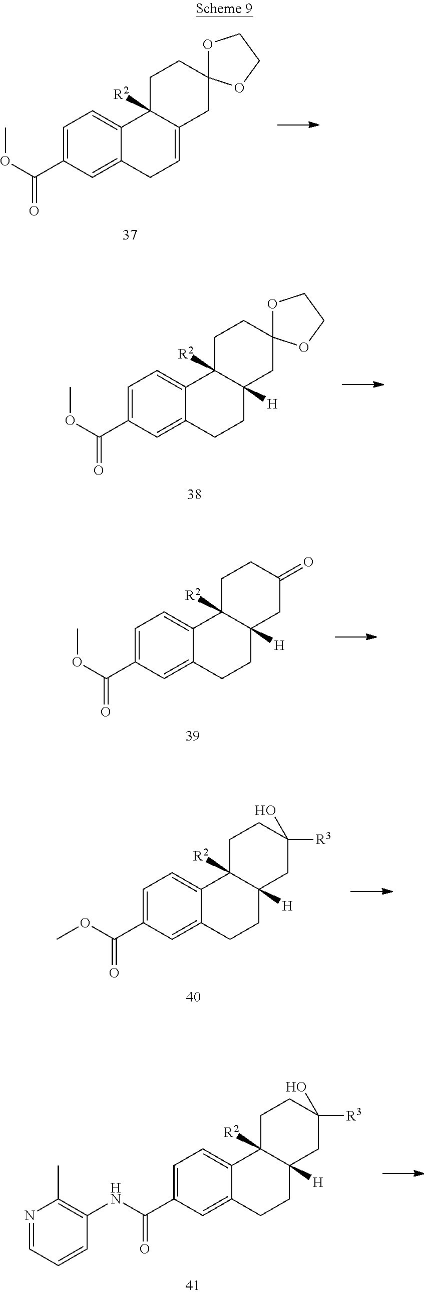

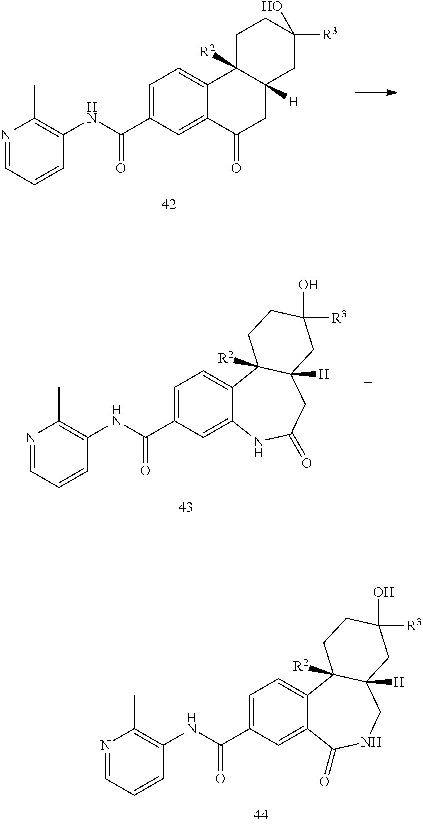

### Examples #21 and 22

### (7aR,11aS)-11a-Benzyl-9-hydroxy-N-(2-methylpyridin-3-yl)-6-oxo-9-(trifluoromethyl)-6,7,7a,8,9,10,11,11a-octahydro-5H-dibenzo[b,d]azepine-3-carboxamide (43, R²=Benzyl, R³=Trifluoromethyl) and (4aS,11bS)-11b-benzyl-3-hydroxy-N-(2-methylpyridin-3-yl)-7-oxo-3-(trifluoromethyl)-2,3,4,4a,5,6,7,11b-octahydro-1H-dibenzo[c,e]azepine-9-carboxamide (44, R²=Benzyl, R³=Trifluoromethyl)

### Step #1: (4a′S,10a′S)-Methyl 4a′-benzyl-3′,4′,4a′,9′,10′,10a′-hexahydro-1′H-spiro[[1,3]dioxolane-2,2′-phenanthrene]-7′-carboxylate (38, R²=Benzyl)

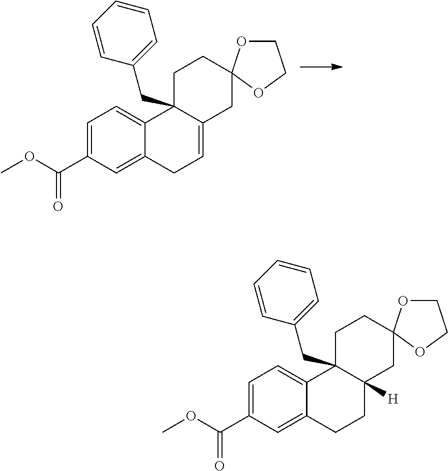

To a solution of (S)-methyl 4a′-benzyl-3′,4′,4a′,9′-tetrahydro-1′H-spiro[[1,3]dioxolane-2,2′-phenanthrene]-7′-carboxylate (37, R²=Benzyl) (60.5 g, 155 mmol; prepared as described in WO 2008093236 A1) and toluene (400 mL) (dried over molecular sieves) was added 20% palladium hydroxide on carbon (10.9 g), washed with acetone (2×20 mL) then toluene (2×20 mL) and then added as a slurry in toluene (20 mL). The mixture was placed under hydrogen (60 psi) in an autoclave at about 50° C. for approximately 20 h. The mixture was cooled to rt, the hydrogen gas was evacuated, and then the mixture was filtered through Celite® with the aid of toluene. The volatiles were removed under reduced pressure to give 35.3 g of a 62 to 38 mixture of trans to cis isomers (based on analytical HPLC). The crude material was purified via preparative chiral HPLC utilizing a Daicel® IA column (20×250 mm) and isocratic elution with 15% hepatane (0.12% DEA modifier) in isopropanol. Fractions containing the cis isomer were combined and concentrated to afford (4a′S,10a′S)-methyl 4a′-benzyl-3′,4′,4a′,9′,10′,10a′-hexahydro-1′H-spiro[[1,3]dioxolane-2,2′-phenanthrene]-7′-carboxylate (38, R²=Benzyl) (11.1 g, 18%) as a solid. LC/MS, method 2, Rₜ=3.01 min, MS m/z 393 (M+H)⁺. ¹H NMR (400 MHz, CDCl₃) δ 7.80 (d, J=1.8 Hz, 1H), 7.63 (dd, J=8.2, 1.9 Hz, 1H), 7.07-7.20 (m, 3H), 6.75-6.79 (m, 3H), 3.90-3.70 (m, 7H), 2.95-2.82 (m, 3H), 2.76 (d, J=13.1 Hz, 1H), 2.35-2.49 (m, 2H), 2.19-2.08 (m, 1H), 1.82-1.50 (m, 5H), 1.35-1.22 (m, 1H)

### Step #2: (4bS,8aS)-Methyl 4b-benzyl-7-oxo-4b,5,6,7,8,8a,9,10-octahydrophenanthrene-2-carboxylate (39, R²=Benzyl)

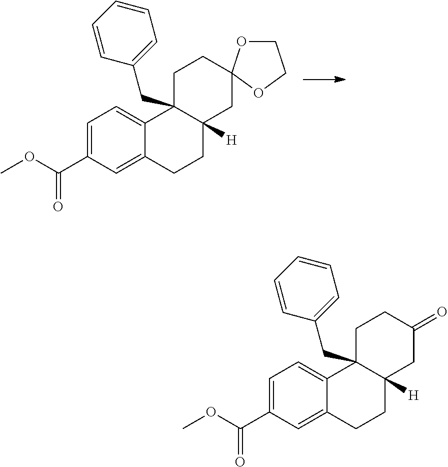

A 500 mL round bottom flask equipped with a nitrogen inlet adapter was charged with (4a′S,10a′S)-methyl 4a′-benzyl-3′,4′,4a′,9′,10′,10a′-hexahydro-1′H-spiro[[1,3]dioxolane-2,2′-phenanthrene]-7′-carboxylate (38, R²=Benzyl) (11.9 g, 30.2 mmol), water (27.2 mL, 1.51 mol), and Tfa (11.6 mL, 151 mmol) in DCM (151 mL) to give a colorless solution. The resulting solution was allowed to stir at rt for about 2 days. The reaction mixture was concentrated and purified via silica gel chromatography eluting with 5%-50% EtOAc in heptane. The fractions containing product were combined and concentrated under reduced pressure to afford (4bS,8aS)-methyl 4b-benzyl-7-oxo-4b,5,6,7,8,8a,9,10-octahydrophenanthrene-2-carboxylate (39, R²=Benzyl) (10.4 g, 99%) as an oil. LC/MS, method 1, Rₜ=0.79 min, MS m/z 366 (M+NH₄)⁺. ¹H NMR (400 MHz, DMSO-d₆) δ 7.76-7.69 (m, 2H), 7.49-7.44 (m, 1H), 7.21-7.14 (m, 3H), 6.95-6.89 (m, 2H), 3.83 (s, 3H), 3.10 (s, 2H), 2.91-2.68 (m, 2H), 2.46-2.24 (m, 3H), 2.22-1.87 (m, 5H), 1.61-1.50 (m, 1H).

### Step #3: (4bS,8aS)-Methyl 4b-benzyl-7-hydroxy-7-(trifluoromethyl)-4b,5,6,7,8,8a,9,10-octahydrophenanthrene-2-carboxylate (40, R²=Benzyl, R³=Trifluoromethyl)

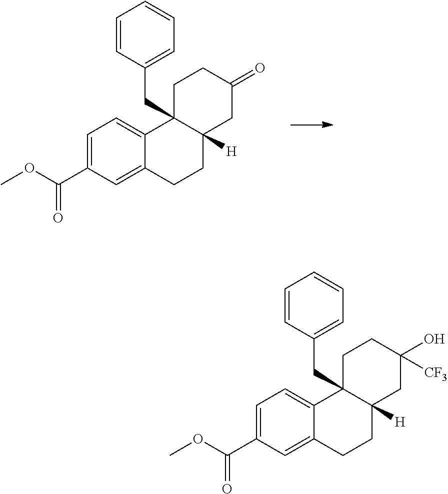

A 250 mL round bottom flask equipped with a nitrogen inlet adapter and a thermometer was charged with (4bS,8aS)-methyl 4b-benzyl-7-oxo-4b,5,6,7,8,8a,9,10-octahydrophenanthrene-2-carboxylate (39, R²=Benzyl) (2.77 g, 7.95 mmol) and trimethyl-trifluoromethyl-silane (2.48 mL, 15.9 mmol) in THF (26.5 mL) to give a colorless solution. The reaction mixture was cooled at about −20° C. (internal temp) for about 30 min. TBAF (2.39 mL, 2.39 mmol) was added dropwise over about 10 min while maintaining an internal temperature range between about −22° C. to −18° C. The reaction mixture was allowed to warm to rt slowly over approximately 2 h. The solution was concentrated to give an oil, deposited onto silica gel and purified via silica gel chromatography eluting with 10% EtOAc in heptane. The fractions containing the desired product were combined and concentrated to afford (4bS,8aS)-methyl 4b-benzyl-7-hydroxy-7-(trifluoromethyl)-4b,5,6,7,8,8a,9,10-octahydrophenanthrene-2-carboxylate (40, R²=Benzyl, R³=Trifluoromethyl) (2.40 g, 72%) as a white solid. LC/MS, method 2, Rₜ=2.80 min, MS m/z 436 (M+NH₄)⁺. ¹H NMR (400 MHz, CDCl₃) δ 7.82 (dd, J=8.3, 1.9 Hz, 1H), 7.73 (d, J=1.9 Hz, 1H), 7.31 (d, J=8.3 Hz, 1H), 7.16-7.06 (m, 3H), 6.71 (t, J=1.6 Hz, 2H), 3.91 (s, 3H), 3.07 (bs, 2H), 2.82-2.72 (m, 1H), 2.65-2.53 (m, 1H), 2.22-2.04 (m, 3H), 2.04-1.78 (m, 4H), 1.70 (s, 1H), 1.66-1.57 (m, 2H).

### Step #4: (4bS,8aS)-4b-Benzyl-7-hydroxy-N-(2-methylpyridin-3-yl)-7-(trifluoromethyl)-4b,5,6,7,8,8a,9,10-octahydrophenanthrene-2-carboxamide (41, R²=Benzyl, R³=Trifluoromethyl)

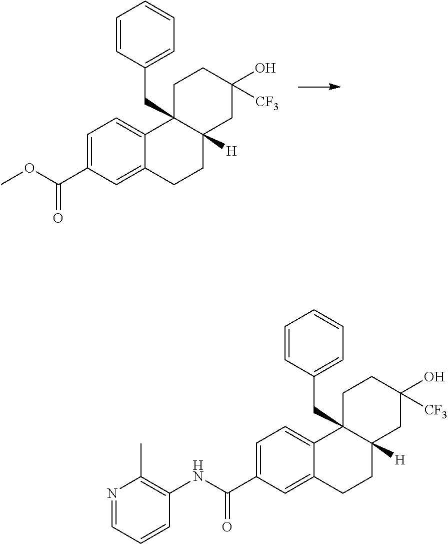

A 50 mL round bottom flask equipped with a septa cap outfitted with nitrogen inlet needle was charged with 2-methylpyridin-3-amine (0.465 g, 4.30 mmol) and (4bS,8aS)-methyl 4b-benzyl-7-hydroxy-7-(trifluoromethyl)-4b,5,6,7,8,8 a,9,10-octahydrophenanthrene-2-carboxylate (40, R²=Benzyl, R³=Trifluoromethyl) (1.20 g, 2.87 mmol) in toluene (14.3 mL) to give a colorless solution. LiHMDS (8.60 mL, 8.60 mmol) (1M solution in THF) was added slowly via syringe. The resulting suspension was allowed to stir at rt for about 2 h and then treated with an excess of water (slow addition). The mixture was extracted with EtOAc and the organic phase was separated and washed with water, saturated aqueous NaCl solution, dried over MgSO₄, filtered and concentrated under reduced pressure. The resulting sample was purified via silica gel chromatography eluting with 2 to 5% MeOH in EtOAc. The fractions containing the desired product were combined and concentrated under reduced pressure to afford (4bS,8aS)-4b-benzyl-7-hydroxy-N-(2-methylpyridin-3-yl)-7-(trifluoromethyl)-4b,5,6,7,8,8a,9,10-octahydrophenanthrene-2-carboxamide (41, R²=Benzyl, R³=Trifluoromethyl) (1.27 g, 90%) as a solid. LC/MS, method 2, Rₜ=2.41 min, MS m/z 495 (M+H)⁺. ¹H NMR (400 MHz, DMSO-d₆) δ 9.97 (s, 1H), 8.33 (dd, J=4.7, 1.6 Hz, 1H), 7.81 (dd, J=8.2, 1.9 Hz, 1H), 7.73 (dd, J=8.0, 1.6 Hz, 1H), 7.66 (d, J=1.9 Hz, 1H), 7.63 (d, J=8.2 Hz, 1H), 7.27 (dd, J=8.0, 4.7 Hz, 1H), 7.17-7.07 (m, 3H), 6.86-6.80 (m, 2H), 3.31 (s, 2H), 3.15 (d, J=13.9 Hz, 1H), 3.04 (d, J=13.9 Hz, 1H), 2.79-2.65 (m, 1H), 2.44 (s, 3H), 2.17-1.78 (m, 7H), 1.62-1.49 (m, 2H).

### Step #5: (4bS,8aR)-4b-Benzyl-7-hydroxy-N-(2-methylpyridin-3-yl)-10-oxo-7-(trifluoromethyl)-4b,5,6,7,8,8 a,9,10-octahydrophenanthrene-2-carboxamide (42, R²=Benzyl, R³=Trifluoromethyl)

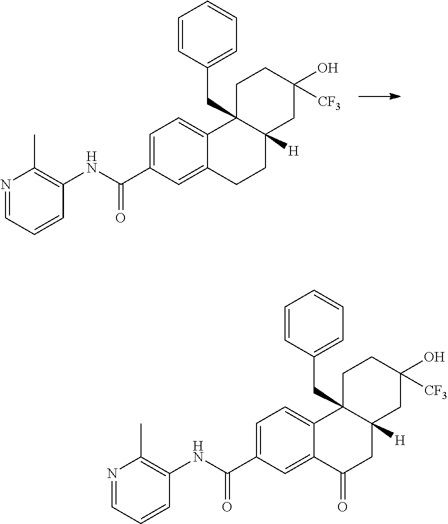

A 250 mL round bottom flask equipped with a septa cap outfitted with a pipette adapter was charged with (4bS,8aS)-4b-benzyl-7-hydroxy-N-(2-methylpyridin-3-yl)-7-(trifluoromethyl)-4b,5,6,7,8,8 a,9,10-octahydrophenanthrene-2-carb oxamide (41, R²=Benzyl, R³=Trifluoromethyl) (0.875 g, 1.77 mmol) in DCM (15.9 mL) and MeOH (1.7 mL) to give a colorless solution. The reaction mixture was cooled at approximately −78° C. for about 15 min. Ozone was bubbled through the sample at a rate of approximately 4 psi continuously for about 5 h. The reaction was capped and allowed to warm slowly to rt over approximately 18 h. The sample was treated with a molar excess of PS-PPh₃ for about 2 h. The resulting suspension was filtered and deposited onto silica gel. The sample was purified via silica gel chromatography eluting with 0-10% MeOH in EtOAc. All fractions containing the desired product along with starting material were combined and concentrated to give a solid (680 mg). This residue was dissolved in DCM (15.9 mL) and MeOH (1.7 mL) to give a colorless solution. Ozone gas was bubbled through the sample at a rate of approximately 4 psi for about 5 min at periodic intervals of approximately 1 h to 18 h for an additional 60 h. The sample was treated with a molar excess of PS-PPh₃ for about 2 h. The resulting suspension was filtered and concentrated. The resulting sample was purified via reverse-phase chromatography to give (4bS,8aR)-4b-benzyl-7-hydroxy-N-(2-methylpyridin-3-yl)-10-oxo-7-(trifluoromethyl)-4b,5,6,7,8,8a,9,10-octahydrophenanthrene-2-carboxamide (42, R²=Benzyl, R³=Trifluoromethyl) (0.133 g, 15%) as a solid. LC/MS, method 2, Rₜ=2.14 min, MS m/z 509 (M+H)⁺. ¹H NMR (400 MHz, DMSO-d₆) δ 10.28 (s, 1H), 8.56 (d, J=2.1 Hz, 1H), 8.36 (dd, J=4.7, 1.7 Hz, 2H), 8.20 (dd, J=8.2, 2.2 Hz, 2H), 7.74 (dd, J=7.9, 1.7 Hz, 2H), 7.60-7.53 (m, 1H), 7.32-7.19 (m, 4H), 6.99-6.93 (m, 2H), 5.75 (s, 1H), 3.16 (d, J=13.6 Hz, 1H), 3.02 (d, J=13.6 Hz, 1H), 2.71-2.58 (m, 1H), 2.45 (s, 3H), 2.37-2.27 (m, 2H), 2.11-1.97 (m, 1H), 1.85-1.72 (m, 1H), 1.40-1.28 (m, 1H).

### Step #6: (7aR,11aS)-11a-Benzyl-9-hydroxy-N-(2-methylpyridin-3-yl)-6-oxo-9-(trifluoromethyl)-6,7,7a,8,9,10,11,11a-octahydro-5H-dibenzo[b,d]azepine-3-carboxamide (43, R²=Benzyl, R³=Trifluoromethyl) and (4aS,11bS)-11b-benzyl-3-hydroxy-N-(2-methylpyridin-3-yl)-7-oxo-3-(trifluoromethyl)-2,3,4,4 a,5,6,7,11b-octahydro-1H-dibenzo[c,e]azepine-9-carboxamide (44, R²=Benzyl, R³=Trifluoromethyl)

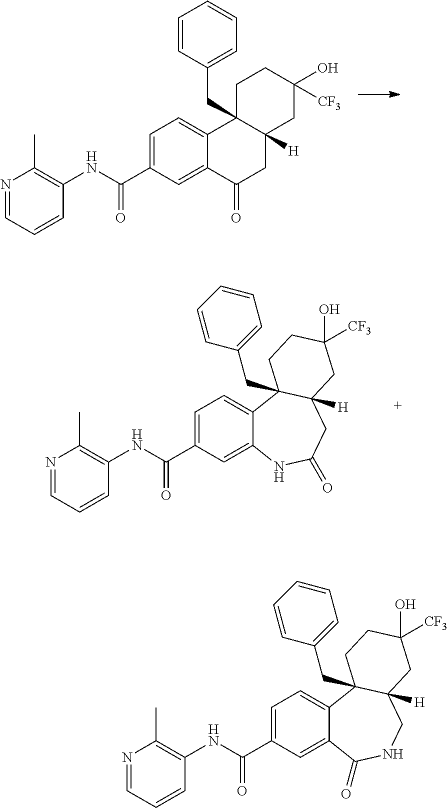

A 10 mL reaction vial equipped with a nitrogen inlet adapter was charged with (4bS,8aR)-4b-benzyl-7-hydroxy-N-(2-methylpyridin-3-yl)-10-oxo-7-(trifluoromethyl)-4b,5,6,7,8,8 a,9,10-octahydrophenanthrene-2-carboxamide (42, R²=Benzyl, R³=Trifluoromethyl) (0.065 g, 0.128 mmol) and sodium azide (0.017 g, 0.26 mmol) in DCM (1.3 mL) to give a suspension. MsOH (0.017 mL, 0.26 mmol) was added in one portion. The resulting solution was allowed to stir at rt for about 1 h and MsOH (0.066 mL, 1.0 mmol) was added in one portion. The resulting solution was allowed to stir at rt for about 18 h. The reaction mixture was partitioned between DCM and water. The organic phase was washed with saturated aqueous NaHCO₃ (2×50 mL), water (50 mL), and saturated aqueous NaCl (50 mL). The organic phase was dried over MgSO₄, filtered, and concentrated under reduced pressure to give a sample that was purified by reverse-phase chromatography to give (4aS,11bS)-11b-benzyl-3-hydroxy-N-(2-methylpyridin-3-yl)-7-oxo-3-(trifluoromethyl)-2,3,4,4a,5,6,7,11b-octahydro-1H-dibenzo[c,e]azepine-9-carboxamide (43, R²=Benzyl, R³=Trifluoromethyl) (0.0066 g, 10%) as the first eluting sample and (7aR,11aS)-11a-benzyl-9-hydroxy-N-(2-methylpyridin-3-yl)-6-oxo-9-(trifluoromethyl)-6,7,7a,8,9,10,11,11a-octahydro-5H-dibenzo[b,d]azepine-3-carboxamide (44, R²=Benzyl, R³=Trifluoromethyl) (0.0125 g, 18%) as the second eluting sample. Data for (43, R²=Benzyl, R³=Trifluoromethyl): LC/MS, method 2, Rₜ=1.90 min, MS m/z 524 (M+H)⁺. ¹H NMR (400 MHz, DMSO-d₆) δ 10.04 (s, 1H), 9.72 (s, 1H), 8.34 (dd, J=4.7, 1.6 Hz, 1H), 7.76 (dd, J=8.0, 1.6 Hz, 1H), 7.72-7.65 (m, 1H), 7.59 (d, J=1.9 Hz, 1H), 7.52 (d, J=8.4 Hz, 1H), 7.28 (dd, J=7.9, 4.7 Hz, 1H), 7.09-7.03 (m, 3H), 6.63-6.57 (m, 2H), 5.88 (s, 1H), 3.37 (d, J=13.9 Hz, 1H), 2.86 (d, J=13.9 Hz, 1H), 2.44 (s, 3H), 2.40-1.97 (m, 6H), 1.89 (s, 1H), 1.77-1.67 (m, 1H), 1.57-1.45 (m, 1H). Data for (44, R²=Benzyl, R³=Trifluoromethyl) LC/MS, method 2, Rₜ=1.84 min, no parent ion. ¹H NMR (400 MHz, DMSO-d₆) δ 10.20 (s, 1H), 8.50-8.48 (m, 1H), 8.37-8.28 (m, 2H), 7.93 (dd, J=8.2, 2.0 Hz, 1H), 7.74 (dd, J=8.0, 2.0 Hz, 1H), 7.32-7.23 (m, 2H), 7.07-7.00 (m, 3H), 6.67-6.60 (m, 2H), 5.96 (s, 1H), 3.24-3.15 (m, 1H), 3.18-3.10 (m, 1H), 2.44 (s, 3H), 2.39-2.30 (m, 2H), 2.13-1.97 (m, 3H), 1.90-1.74 (m, 4H).

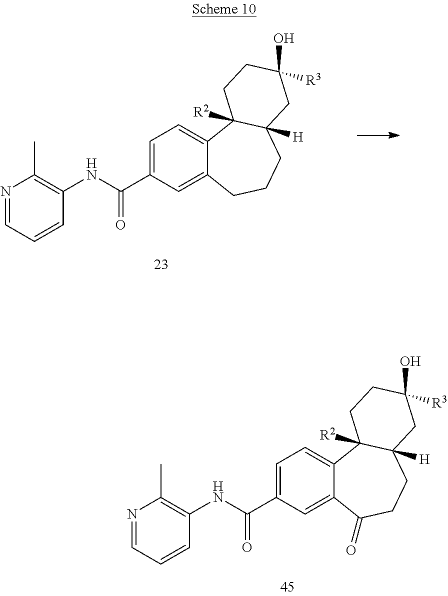

### Example #23

### (7aS,9R,11aS)-11a-Benzyl-9-ethyl-9-hydroxy-5-oxo-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-ethyl-9-hydroxy-5-oxo-6,7,7a,8,9,10,11,11a-octahydro-5H-dibenzo[a,c]cycloheptene-3-carboxylic acid (2-methyl-pyridin-3-yl)-amide (45, R²=Benzyl, R³=Ethyl)

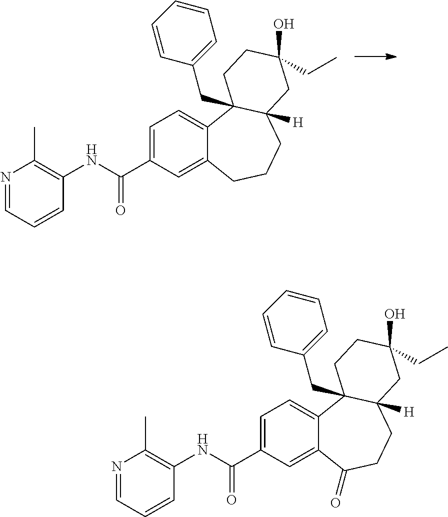

A solution of (7aS,9R,11aS)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide (23, R²=Benzyl and R³=Ethyl) (180 mg, 0.384 mmol), potassium permanganate (0.304 mg, 1.92 mmol) and copper (II) sulfate pentahydrate (480 mg, 1.92 mmol) in DCM (8.5 mL) was treated with water (1.0 mL). The reaction was stirred at rt for about 30 min and then diluted with DCM (15 mL) and treated with saturated aqueous NaHCO₃ (15 mL). The layers were separated and the aqueous phase was extracted with DCM (2×15 mL). The combined organic phases were filtered through a Biotage Isolute® SPE Phase Separator and concentrated under reduced pressure. The residue was chromatographed on a silica gel column (25 g), eluting with a gradient of 0-100% EtOAc in DCM. Collection and concentration of the appropriate fractions gave a clear film to which diethyl Et₂O (5 mL) was added. Concentration gave a white solid, (7aS,9R,11aS)-11a-benzyl-9-ethyl-9-hydroxy-5-oxo-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-ethyl-9-hydroxy-5-oxo-6,7,7a,8,9,10,11,11a-octahydro-5H-dibenzo[a,c]cycloheptene-3-carboxylic acid (2-methyl-pyridin-3-yl)-amide (45, R²=Benzyl, R³=Ethyl) (0.047 g, 25%), LC/MS, method 2, Rₜ=1.97 min, MS m/z 483 (M+H)⁺, ¹H NMR (400 MHz, DMSO-d₆) δ 10.13 (s, 1H), 8.34 (dd, J=4.7, 1.6 Hz, 1H), 7.96 (d, J=2.1 Hz, 1H), 7.82 (dd, J=8.3, 2.2 Hz, 1H), 7.73 (dd, J=8.0, 1.6 Hz, 1H), 7.28 (dd, J=7.9, 4.7 Hz, 1H), 7.11-7.03 (m, 3H), 6.98 (d, J=8.4 Hz, 1H), 6.52 (d, J=2.5 Hz, 2H), 4.04 (s, 1H), 3.04-2.89 (m, 2H), 2.74-2.56 (m, 3H), 2.45-2.41 (m, 5H), 1.78-1.64 (m, 1H), 1.54-1.46 (m, 1H), 1.44-1.18 (m, 6H), 0.75 (t, J=7.4, 3H).

### Examples #24 and #25

### (7aS,9R,11aS)-11a-benzyl-9-ethyl-9-hydroxy-5-oxo-6,7,7a,8,9,10,11,11a-octahydro-5H-dibenzo[a,c]cycloheptene-3-carboxylic acid (2-methyl-pyridin-3-yl)-amide (45, R²=Benzyl, R³=Ethyl) and (7aR,9S,11aR)-11a-benzyl-9-ethyl-9-hydroxy-5-oxo-6,7,7a,8,9,10,11,11a-octahydro-5H-dibenzo[a,c]cycloheptene-3-carboxylic acid (2-methyl-pyridin-3-yl)-amide (45, R²=Benzyl, R³=Ethyl)

The enantiomers were separated by chiral preparative chromatography method 5 to provide first (7aS,9R,11aS)-11a-benzyl-9-ethyl-9-hydroxy-5-oxo-6,7,7a,8,9,10,11,11a-octahydro-5H-dibenzo[a,c]cycloheptene-3-carboxylic acid (2-methyl-pyridin-3-yl)-amide (45 7aS,9R,11aS, R²=Benzyl, R³=Ethyl) (Example 24) and second (7aR,9S,11aR)-11a-benzyl-9-ethyl-9-hydroxy-5-oxo-6,7,7a,8,9,10,11,11a-octahydro-5H-dibenzo[a,c]cycloheptene-3-carboxylic acid (2-methyl-pyridin-3-yl)-amide (45 7aR,9S,11aR, R²=Benzyl, R³=Ethyl) (Example 25).

### Example #26 and 27

### (7aR,9S,11aS)-9-Ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide; compound with (7aS,9R,11aR)-9-ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide (23, R²=Propyl, R³=Ethyl) and (7aR,9R,11aS)-9-ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide; compound with (7aS,9S,11aR)-9-ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide (24, R²=Propyl, R³=Ethyl)

### Step 1: 5-Allyl-2-methoxy-5,7,8,9-tetrahydro-benzocyclohepten-6-one (4, R²=Allyl)

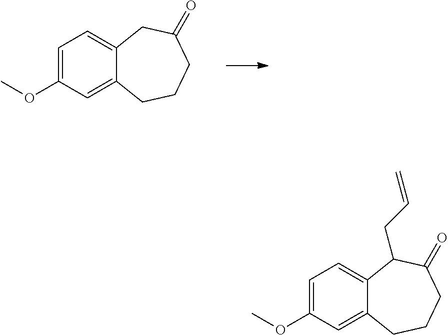

A solution of 2-methoxy-8,9-dihydro-5H-benzo[7]annulen-6(7H)-one (3) (15.0 g, 79 mmol) in toluene (300 mL) was treated with pyrrolidine (13.0 mL, 158 mmol) and the mixture was heated at reflux for about 1 h, removing water by means of a Dean-Stark trap. Additional pyrroline (6.5 mL, 79 mmol) was added and the reaction was refluxed for about an additional 1 h. The reaction was cooled, concentrated under reduced pressure and then re-dissolved in 1,4-dioxane (300 mL) and allyl bromide (15.0 mL, 173 mmol) was added. The mixture was heated at about 70° C. for about 18 h. Additional allyl bromide (15.0 mL, 173 mmol) was added and the reaction continued for about 24 h. The reaction was cooled and concentrated. The residue was taken up in 10% aqueous 1,4-dioxane (300 mL) and stirred at rt for about 1 h. The mixture was diluted with water (300 mL) and extracted with DCM (2×300 mL). The combined extracts were washed with saturated aqueous NaCl (100 mL), dried with Na₂SO₄, filtered and concentrated under reduced pressure. The crude was purified on silica gel (330 g) using a gradient from 5 to 15% EtOAc in heptane. The product containing fractions were combined and concentrated under reduced pressure to an oil which solidifies on standing to yield 5-allyl-2-methoxy-5,7,8,9-tetrahydro-benzocyclohepten-6-one (4, R²=Allyl) (13.3 g, 73%), LC/MS, method 1, Rₜ=1.45 min, MS m/z 231 (M+H)⁺, ¹H NMR (400 MHz, DMSO-d₆) δ 7.02 (d, J=8.1 Hz, 1H), 6.78-6.70 (m, 2H), 5.80-5.66 (m, 1H), 5.10-4.91 (m, 2H), 4.10-4.00 (m, 1H), 3.70 (s, 3H), 3.12-3.00 (m, 1H), 2.84-2.62 (m, 3H), 2.45-2.28 (m, 2H), 2.10-1.98 (m, 1H), 1.69-1.55 (m, 1H).

### Step 2: 2-Methoxy-5-propyl-5,7,8,9-tetrahydro-benzocyclohepten-6-one (4, R²=Propyl)

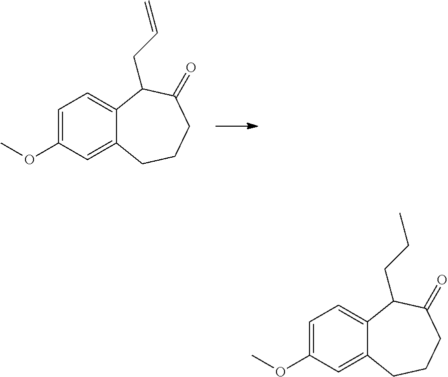

A solution of 5-allyl-2-methoxy-8,9-dihydro-5H-benzo[7]annulen-6(7H)-one (4, R²=Allyl) (1.00 g, 4.34 mmol) in toluene (20 mL) containing 20% Pd(OH)₂ on carbon (0.091 g) was evacuated and hydrogen (about 60 psi) was added. The reaction was shaken for about 2 h, then filtered through Celite® (5 g). The filtrate was concentrated under vacuum to yield 2-methoxy-5-propyl-5,7,8,9-tetrahydro-benzocyclohepten-6-one (4, R²=Propyl) (962 mg, 95%) as a clear oil, LC/MS, method 4, Rₜ=1.74 min, MS m/z 233 (M+H)⁺, ¹H NMR (400 MHz, DMSO-d₆) δ 7.05-7.00 (m, 1H), 6.77-6.71 (m, 2H), 3.92-3.84 (m, 1H), 3.70 (s, 3H), 3.09-2.97 (m, 1H), 2.82-2.72 (m, 1H), 2.72-2.60 (m, 1H), 2.42-2.31 (m, 1H), 2.03-1.95 (m, 2H), 1.71-1.50 (m, 2H), 1.27-1.14 (m, 2H), 0.87 (t, J=7.3 Hz, 3H).

### Step 3: 9-Methoxy-11b-propyl-1,2,5,6,7,11b-hexahydro-dibenzo[a,c]cyclohepten-3-one (5, R²=Propyl)

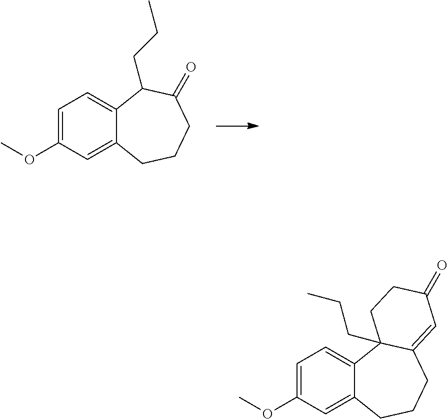

9-Methoxy-11b-propyl-1,2,5,6,7,11b-hexahydro-dibenzo[a,c]cyclohepten-3-one (5, R²=Propyl) was prepared in a manner similar to the preparation of 11b-benzyl-9-methoxy-1,2,5,6,7,11b-hexahydro-dibenzo[a,c]cyclohepten-3-one (5, R²=Benzyl), substituting 2-methoxy-5-propyl-5,7,8,9-tetrahydro-benzocyclohepten-6-one (4, R²=Propyl) for 5-benzyl-2-methoxy-8,9-dihydro-5H-benzo[7]annulen-6(7H)-one (4, R²=Benzyl) to yield 9-methoxy-11b-propyl-1,2,5,6,7,11b-hexahydro-dibenzo[α]cyclohepten-3-one (5, R²=Propyl) (53%) as an off-white solid. LC/MS, method 4, Rₜ=1.73 min, MS m/z 285 (M+H)⁺, ¹H NMR (400 MHz, DMSO-d₆) δ 7.23 (d, J=8.7 Hz, 1H), 6.80 (dd, J=8.7, 2.9 Hz, 1H), 6.70 (d, J=2.8 Hz, 1H), 5.81 (s, 1H), 3.71 (s, 3H), 2.77 (dd, J=12.1, 6.6 Hz, 1H), 2.68-2.56 (m, 1H), 2.55-2.46 (m, 1H), 2.43-2.36 (m, 1H), 2.31-2.21 (m, 2H), 2.21-2.11 (m, 1H), 2.10-2.00 (m, 1H), 1.98-1.87 (m, 1H), 1.85-1.68 (m, 3H), 1.33-1.20 (m, 1H), 1.11-0.98 (m, 1H), 0.86 (m, 3H)

### Step 4: 9-Hydroxy-11b-propyl-1,2,5,6,7,11b-hexahydro-dibenzo[a,c]cyclohepten-3-one (6, R²=Propyl)

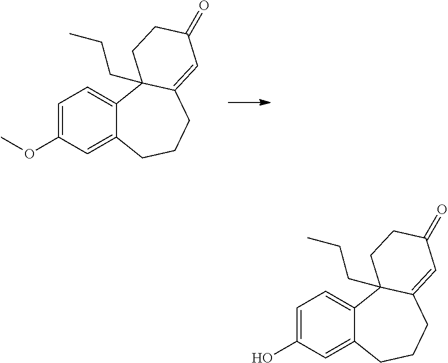

A mixture of 9-methoxy-11b-propyl-1,2,5,6,7,11b-hexahydro-dibenzo[a,c]cyclohepten-3-one (5, R²=Propyl) (620 mg, 2.18 mmol) and DL-methionone (1.06 g, 7.09 mmol) in methanesulfonic acid (12 mL, 185 mmol) was allowed to stir under nitrogen at rt for about 48 h. The mixture was diluted with DCM (100 mL) and poured carefully into ice water (100 mL). The product was extracted with DCM (2×100 mL). The combined organic layers were washed with water (100 mL), dried over Na₂SO₄, filtered and concentrated under reduced pressure. The residue was purified on silica gel (40 g) using a gradient from 20-50% EtOAc in heptane. Product containing fractions were combined and concentrated under reduced pressure. The residue was further dried under vacuum to constant weight to yield 9-hydroxy-11b-propyl-1,2,5,6,7,11b-hexahydro-dibenzo[a,c]cyclohepten-3-one (6, R²=Propyl) as an off white solid (565 mg, 96%). LC/MS, method 4, Rₜ=1.25 min, MS m/z 269 (M−H)⁻. ¹H NMR (400 MHz, DMSO-d₆) δ 9.19 (s, 1H), 7.10 (d, J=8.6 Hz, 1H), 6.62 (dd, J=8.5, 2.7 Hz, 1H), 6.51 (d, J=2.7 Hz, 1H), 5.79 (s, 1H), 2.79-2.68 (m, 1H), 2.68-2.54 (m, 1H), 2.44-2.33 (m, 2H), 2.29-2.17 (m, 2H), 2.17-1.99 (m, 2H), 1.96-1.84 (m, 1H), 1.84-1.66 (m, 3H), 1.33-1.19 (m, 1H), 1.11-0.96 (m, 1H), 0.86 (t, J=7.2 Hz, 3H).

### Step 5: 11b-Benzyl-9-hydroxy-1,2,4,4 a,5,6,7,11b-octahydro-dibenzo[a,c]cyclohepten-3-one (7, R²=Propyl)

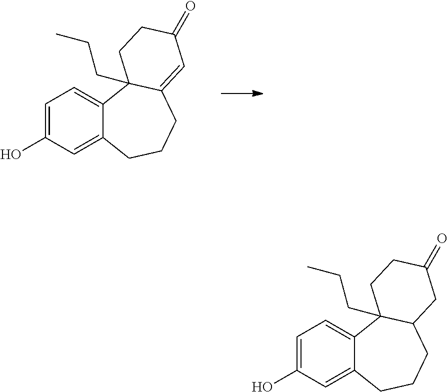

A mixture of 9-methoxy-11b-propyl-1,2,5,6,7,11b-hexahydro-dibenzo[a,c]cyclohepten-3-one (6, R²=Propyl) (563 mg, 2.08 mmol) and 20% Pd(OH)₂ on carbon (146 mg) in EtOH (10 mL) was shaken under about 40 psi hydrogen at rt for about 3 h. The catalyst was removed by filtration through a pad of Celite®, rinsing with EtOAc (3×10 mL) and the filtrate was concentrated under reduced pressure. The residue, (7, R²=Propyl), was used in the next step without further purification.

### Step 6: Trifluoro-methanesulfonic acid (7aS,11aR)-9-oxo-11a-propyl-6,7,7a,8,9,10,11,11a-octahydro-5H-dibenzo[a,c]cyclohepten-3-yl ester (9, R²=Propyl)

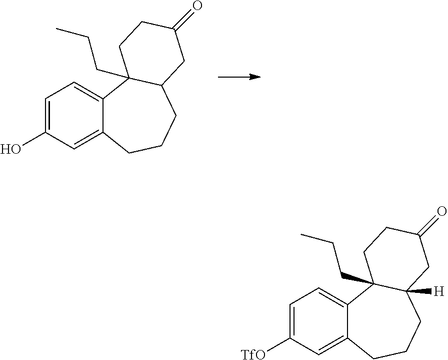

A slurry of 11b-benzyl-9-hydroxy-1,2,4,4a,5,6,7,11b-octahydro-dibenzo[a,c]cyclohepten-3-one (7, R²=Benzyl) (560 mg, 2.06 mmol) in DCM (5.0 mL) was treated with N-phenylbis(trifluoromethanesulfonimide (734 mg, 2.06 mmol) and (DIEA 90.7 mL, 4.11 mmol) at rt and stirred about 18 h. The reaction was concentrated under reduced pressure. The residue was purified on silica gel (25 g) using a gradient from 10 to 30% EtOAc in heptane. Product containing fractions were combined and concentrated to yield trifluoro-methanesulfonic acid (7aS,11aR)-9-oxo-11a-propyl-6,7,7a,8,9,10,11,11a-octahydro-5H-dibenzo[a,c]cyclohepten-3-yl ester (9, R²=Propyl) as a colorless oil (232 mg, 28%). LC/MS, method 2, Rₜ=3.21 min, MS m/z 463 (M+OAc)⁻. ¹H NMR (400 MHz, DMSO-d₆) δ 7.48 (d, J=8.9 Hz, 1H), 7.28 (d, J=2.9 Hz, 1H), 7.24 (dd, J=8.8, 2.9 Hz, 1H), 3.09-2.98 (m, 1H), 2.93-2.83 (m, 1H), 2.71-2.60 (m, 1H), 2.44-2.33 (m, 1H), 2.32-2.23 (m, 1H), 2.23-2.02 (m, 4H), 1.96-1.87 (m, 1H), 1.80-1.64 (m, 2H), 1.65-1.53 (m, 1H), 1.51-1.33 (m, 2H), 1.35-1.19 (m, 1H), 0.82-0.62 (m, 4H).

### Step 7: (7aS,11aR)-9-Oxo-11a-propyl-6,7,7a,8,9,10,11,11a-octahydro-5H-dibenzo[a,c]cycloheptene-3-carboxylic acid methyl ester; compound with (7aR,11aS)-9-oxo-11a-propyl-6,7,7a,8,9,10,11,11a-octahydro-5H-dibenzo[a,c]cycloheptene-3-carboxylic acid methyl ester (21, R²=Propyl)

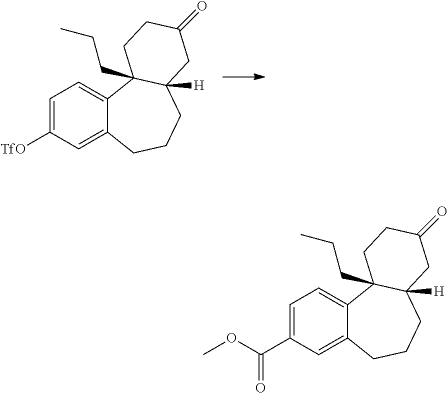

Compound 21 (R²=Propyl) was prepared in a manner similar to the preparation of Compound 21 (R²=Benzyl) substituting trifluoro-methanesulfonic acid (7aS,11aR)-9-oxo-11a-propyl-6,7,7a,8,9,10,11,11a-octahydro-5H-dibenzo[a,c]cyclohepten-3-yl ester; compound with trifluoro-methanesulfonic acid (7aR,11aS)-9-oxo-11a-propyl-6,7,7a,8,9,10,11,11a-octahydro-5H-dibenzo[a,c]cyclohepten-3-yl ester (9, R²=Propyl) for trifluoro-methanesulfonic acid (7aS,11aS)-11a-benzyl-9-oxo-6,7,7a,8,9,10,11,11a-octahydro-5H-dibenzo[a,c]cyclohepten-3-yl ester; compound with trifluoro-methanesulfonic acid (7aR,11aR)-11a-benzyl-9-oxo-6,7,7a,8,9,10,11,11a-octahydro-5H-dibenzo[a,c]cyclohepten-3-yl ester (9, R²=Phenyl) to yield (7aS,11aR)-9-oxo-11a-propyl-6,7,7a,8,9,10,11,11a-octahydro-5H-dibenzo[a,c]cycloheptene-3-carboxylic acid methyl ester; compound with (7aR,11aS)-9-oxo-11a-propyl-6,7,7a,8,9,10,11,11a-octahydro-5H-dibenzo[a,c]cycloheptene-3-carboxylic acid methyl ester (21, R²=Propyl) (800 mg, 68%) as a white solid. LC/MS, method 1, Rₜ=1.56 min, no parent ion (M+H)⁺. ¹H NMR (400 MHz, DMSO-d₆) δ 7.79-7.72 (m, 2H), 7.48 (d, J=8.0 Hz, 1H), 3.83 (s, 3H), 3.11-3.00 (m, 1H), 2.85-2.76 (m, 1H), 2.76-2.60 (m, 1H), 2.44-2.38 (m, 1H), 2.33-2.09 (m, 4H), 2.05-1.98 (m, 1H), 1.93-1.88 (m, 1H), 1.82-1.21 (m, 7H), (t, J=7.0 Hz, 3H).

### Step 8: (7aS,11aR)-9-Oxo-11a-propyl-6,7,7a,8,9,10,11,11a-octahydro-5H-dibenzo[a,c]cycloheptene-3-carboxylic acid (2-methyl-pyridin-3-yl)-amide; compound with (7aR,11aS)-9-oxo-11a-propyl-6,7,7a,8,9,10,11,11a-octahydro-5H-dibenzo[a,c]cycloheptene-3-carboxylic acid (2-methyl-pyridin-3-yl)-amide (22, R²=Propyl)

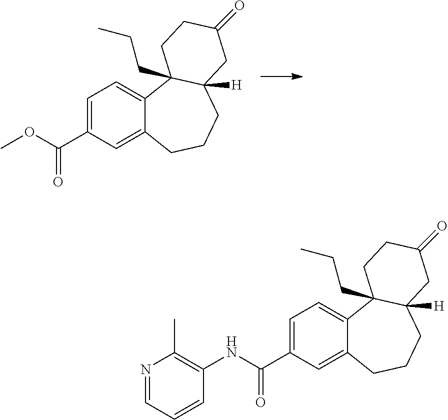

Compound 22 (R²=Propyl) was prepared in a manner similar to the preparation of Compound 22 (R²=Benzyl), substituting (7aS,11aR)-9-oxo-11a-propyl-6,7,7a,8,9,10,11,11a-octahydro-5H-dibenzo[a,c]cycloheptene-3-carboxylic acid methyl ester; compound with (7aR,11aS)-9-oxo-11a-propyl-6,7,7a,8,9,10,11,11a-octahydro-5H-dibenzo[a,c]cycloheptene-3-carboxylic acid methyl ester (21, R²=Propyl) for (7aR,11aR)-11a-benzyl-9-oxo-6,7,7a,8,9,10,11,11a-octahydro-5H-dibenzo[a,c]cycloheptene-3-carboxylic acid methyl ester; compound with (7aS,11aS)-11a-benzyl-9-oxo-6,7,7a,8,9,10,11,11a-octahydro-5H-dibenzo[a,c]cycloheptene-3-carboxylic acid methyl ester (21, R²=Phenyl) to yield (7aS,11aR)-9-oxo-11a-propyl-6,7,7a,8,9,10,11,11a-octahydro-5H-dibenzo[a,c]cycloheptene-3-carboxylic acid (2-methyl-pyridin-3-yl)-amide; compound with (7aR,11aS)-9-oxo-11a-propyl-6,7,7a,8,9,10,11,11a-octahydro-5H-dibenzo[a,c]cycloheptene-3-carboxylic acid (2-methyl-pyridin-3-yl)-amide (22, R²=Propyl) (36%) as a white solid. LC/MS, method 1, Rₜ=1.37 min, MS m/z 391 (M+H)⁺, ¹H NMR (400 MHz, DMSO-d₆) δ 9.99 (s, 1H), 8.33 (dd, J=4.7, 1.6 Hz, 1H), 7.83-7.76 (m, 2H), 7.72 (dd, J=7.9, 1.4 Hz, 1H), 7.49 (d, J=8.2 Hz, 1H), 7.27 (dd, J=7.9, 4.7 Hz, 1H), 3.16-3.04 (m, 1H), 2.97-2.92 (m, 1H), 2.82-2.71 (m, 1H), 2.46 (s, 3H), 2.35-2.01 (m, 4H), 2.01-1.68 (m, 3H), 1.67-1.17 (m, 4H), 0.83-0.69 (m, 4H).

### Step #9: (7aR,9S,11aS)-9-Ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide; compound with (7aS,9R,11aR)-9-ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide (23, R²=Propyl, R³=Ethyl) and (7aR,9R,11aS)-9-ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide; compound with (7aS,9S,11aR)-9-ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide (24, R²=Propyl, R³=Ethyl)

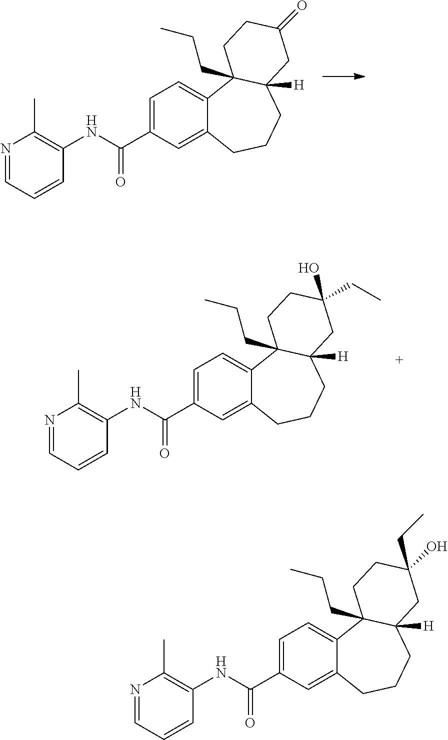

The compounds 23 (R²=Propyl, R³=Ethyl) and 24 (R²=Propyl, R³=Ethyl) were prepared in a manner similar to the preparation of 23 (R²=Benzyl, R³=Ethyl) and 24 (R²=Benzyl, R³=Ethyl) substituting (7aS,11aR)-9-oxo-11a-propyl-6,7,7a,8,9,10,11,11a-octahydro-5H-dibenzo[a,c]cycloheptene-3-carboxylic acid (2-methyl-pyridin-3-yl)-amide; compound with (7aR,11aS)-9-oxo-11a-propyl-6,7,7a,8,9,10,11,11a-octahydro-5H-dibenzo[a,c]cycloheptene-3-carboxylic acid (2-methyl-pyridin-3-yl)-amide (22, R²=Propyl) for (7aS,11aS)-11a-benzyl-9-oxo-6,7,7a,8,9,10,11,11a-octahydro-5H-dibenzo[a,c]cycloheptene-3-carboxylic acid (2-methyl-pyridin-3-yl)-amide; compound with (7aR,11aR)-11a-benzyl-9-oxo-6,7,7a,8,9,10,11,11a-octahydro-5H-dibenzo[a,c]cycloheptene-3-carboxylic acid (2-methyl-pyridin-3-yl)-amide (22, R²=Benzyl) to yield (7aR,9S,11aS)-9-ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide; compound with (7aS,9R,11aR)-9-ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide (23, R²=Propyl, R³=Ethyl) (30%) as a white solid, LC/MS method 3, Rₜ=2.31 min, MS m/z 421 (M+H)⁺, ¹H NMR (400 MHz, DMSO-d₆) δ 9.93 (s, 1H), 8.33 (dd, J=4.7, 1.7 Hz, 1H), 7.81 (d, J=1.9 Hz, 1H), 7.74 (dd, J=8.0, 1.5 Hz, 1H), 7.55 (dd, J=8.2, 1.9 Hz, 1H), 7.27 (dd, J=7.9, 4.8 Hz, 1H), 7.10-7.01 (m, 3H), 6.82 (d, J=8.5 Hz, 1H), 6.63-6.53 (m, 2H), 3.88 (s, 1H), 3.58 (d, J=12.9 Hz, 1H), 3.31-3.24 (m, 1H), 3.07-2.96 (m, 1H), 2.65-2.55 (m, 1H), 2.47-2.36 (m, 5H), 1.95-1.65 (m, 3H), 1.69-1.22 (m, 4H), 1.22-1.01 (m, 4H), 0.71 (t, J=7.3 Hz, 3H) and 7aR,9R,11aS)-9-ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide; compound with (7aS,9S,11aR)-9-ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide (24 R²=Propyl, R³=Ethyl) (4%) LC/MS, method 3, Rₜ=2.62 min, MS m/z 421 (M+H)⁺, ¹H NMR (400 MHz, DMSO-d₆) δ 9.95 (s, 1H), 8.33 (dd, J=4.7, 1.5 Hz, 1H), 7.83-7.77 (m, 1H), 7.74 (dd, J=8.0, 1.4 Hz, 1H), 7.59 (d, J=6.8 Hz, 1H), 7.27 (dd, J=7.9, 4.7 Hz, 1H), 7.13-7.01 (m, 3H), 6.91-6.79 (m, 1H), 6.62-6.54 (m, 2H), 3.89 (s, 1H), 3.55 (d, J=12.7 Hz, 1H), 3.30-3.13 (m, 1H), 3.08-2.89 (m, 1H), 2.59 (d, J=13.0 Hz, 1H), 2.47-2.29 (m, 4H), 2.04-1.73 (m, 3H), 1.73-1.35 (m, 7H), 1.24-1.16 (m, 2H), 0.81 (t, J=7.3 Hz, 3H).

### Example #28

### (3S,4aS,11bS)-11b-Benzyl-3-prop-1-ynyl-2,3,4,4a,5,6,7,11b-octahydro-1H-dibenzo[a,c]cycloheptene-3,9-diol; compound with (3R,4aR,11bR)-11b-benzyl-3-prop-1-ynyl-2,3,4,4a,5,6,7,11b-octahydro-1H-dibenzo[a,c]cycloheptene-3,9-diol (16, R²=Benzyl, R³=1-Propynyl)

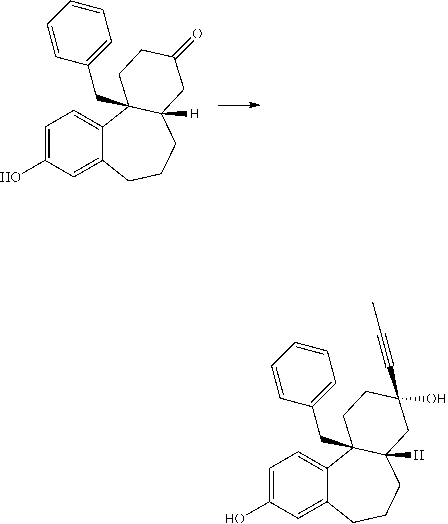

To a stirring solution of THF (1.0 mL) saturated with propyne gas at about 0° C. was added a solution of LDA (0.702 mL, 1.40 mmol) in heptane/THF/ethylbenzene and the mixture was stirred for about 20 min under nitrogen. A solution of (4aS,11bS)-11b-benzyl-9-hydroxy-1,2,4,4a,5,6,7,11b-octahydro-dibenzo[a,c]cyclohepten-3-one; compound with (4aR,11bR)-11b-benzyl-9-hydroxy-1,2,4,4a,5,6,7,11b-octahydro-dibenzo[a,c]cyclohepten-3-one (13, R²=Benzyl) (45 mg, 0.14 mmol) in THF (1.0 mL) was added dropwise and the mixture was stirred about 30 min at about 0° C., allowed to warm to rt and stirred about an additional 1 h. The reaction was quenched by addition of a saturated aqueous NH₄Cl (10 mL) and extracted with EtOAc (3×10 mL). The combined organic extracts were dried over Na₂SO₄, filtered and concentrated. The product was precipitated from EtOAc and heptane to yield (3S,4aS,11bS)-11b-benzyl-3-prop-1-ynyl-2,3,4,4a,5,6,7,11b-octahydro-1H-dibenzo[a,c]cycloheptene-3,9-diol; compound with (3R,4aR,11bR)-11b-benzyl-3-prop-1-ynyl-2,3,4,4a,5,6,7,11b-octahydro-1H-dibenzo[a,c]cycloheptene-3,9-diol (16, R²=Benzyl, R³=1-Propynyl) (38 mg, 75%) as a white solid, LC/MS, method 1, Rₜ=0.85 min, MS m/z 359 (M−H)⁻. ¹H NMR (400 MHz, DMSO-d₆) δ 9.05 (s, 1H), 7.08-6.98 (m, 3H), 6.59-6.48 (m, 3H), 6.41-6.28 (m, 2H), 3.48 (d, J=12.7 Hz, 1H), 5.09 (s, 1H), 3.13-3.03 (m, 1H), 2.82-2.67 (m, 1H), 2.47-2.29 (m, 2H), 2.18-2.05 (m, 1H), 1.96-1.86 (m, 1H), 1.82 (s, 3H), 1.80-1.31 (m, 8H).

### Example #29

### (7aS,9S,11aS)-11a-Benzyl-9-hydroxy-9-prop-1-ynyl-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-11a-benzyl-9-hydroxy-9-prop-1-ynyl-6,7,7a,8,9,10,11,11a-octahydro-5H-dibenzo[a,c]cycloheptene-3-carboxylic acid (2-methyl-pyridin-3-yl)-amide (24, R²=Benzyl, R³=1-Propynyl)

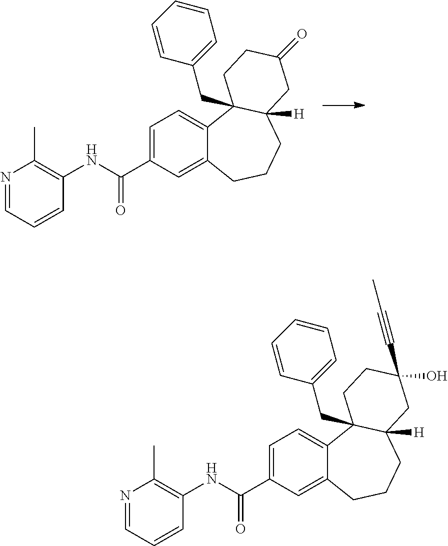

To a stirring solution of THF (2.0 mL) saturated with propyne gas at about 0° C. was added a solution of LDA (1.14 mL, 2.28 mmol) in heptane/THF/ethylbenzene and the mixture was stirred about 20 min under nitrogen. A suspension of (7aS,11aS)-11a-benzyl-9-oxo-6,7,7a,8,9,10,11,11a-octahydro-5H-dibenzo[a,c]cycloheptene-3-carboxylic acid (2-methyl-pyridin-3-yl)-amide; compound with (7aR,11aR)-11a-benzyl-9-oxo-6,7,7a,8,9,10,11,11a-octahydro-5H-dibenzo[a,c]cycloheptene-3-carboxylic acid (2-methyl-pyridin-3-yl)-amide (22, R²=Benzyl) (100 mg, 0.228 mmol) in THF (4 mL) was added dropwise and the mixture was stirred about 30 min at about 0° C., allowed to warm to rt and stirred about an additional 1 h. The reaction was quenched by addition of saturated aqueous NH₄Cl (10 mL) and extracted with EtOAc (3×10 mL). The combined organic extracts were dried over Na₂SO₄, filtered and concentrated. The residue was purified by HPLC on C18 using a gradient from 20 to 100% MeCN in 50 mM NH₄OAc buffer to yield (7aS,9S,11aS)-11a-Benzyl-9-hydroxy-9-prop-1-ynyl-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-11a-benzyl-9-hydroxy-9-prop-1-ynyl-6,7,7a,8,9,10,11,11a-octahydro-5H-dibenzo[a,c]cycloheptene-3-carboxylic acid (2-methyl-pyridin-3-yl)-amide (24, R²=Benzyl, R³=1-Propynyl) (78 mg, 71%) as a white solid, LC/MS, method 1, Rₜ=0.82 min, MS m/z 479 (M+H)⁺, ¹H NMR (400 MHz, DMSO-d₆) δ 9.95 (s, 1H), 8.36-8.29 (m, 1H), 7.84-7.78 (m, 1H), 7.76-7.70 (m, 1H), 7.60-7.52 (m, 1H), 7.30-7.22 (m, 1H), 7.13-7.01 (m, 3H), 6.85-6.75 (m, 1H), 6.63-6.56 (m, 2H), 5.07 (s, 1H), 3.64 (d, J=13.1 Hz, 1H), 3.30-3.20 (m, 1H), 3.08-2.94 (m, 1H), 2.63 (d, J=13.1 Hz, 1H), 2.48-2.40 (m, 4H), 2.28-2.19 (m, 1H), 2.13-2.02 (m, 1H), 1.84 (s, 3H), 1.81-1.38 (m, 7H), 1.34-1.22 (m, 1H).

### Example #30

### (7aS,9R,11aS)-11a-Benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide (23, R²=Benzyl, R³=Methyl)

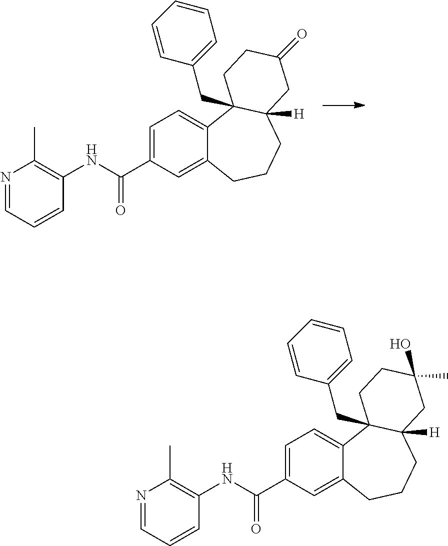

Compound 23 (R²=Benzyl, R³=Methyl) was prepared in a manner similar to the preparation of Compound 23 (R²=Benzyl, R³=Ethyl), substituting methylmagnesium bromide for ethylmagnisium bromide to yield (7aR,9S,11aR)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide; compound with (7aS,9R,11aS)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide (23, R²=Benzyl, R³=Methyl) (70 mg, 45%) as a white solid. LC/MS, method 1, Rₜ=0.75 min, MS m/z 455 (M+H)⁺, ¹H NMR (400 MHz, DMSO-d₆) δ 9.94 (s, 1H), 8.33 (dd, J=4.7, 1.6 Hz, 1H), 7.81 (d, J=2.1 Hz, 1H), 7.74 (dd, J=8.0, 1.6 Hz, 1H), 7.55 (dd, J=8.2, 2.1 Hz, 1H), 7.27 (dd, J=7.9, 4.7 Hz, 1H), 7.10-7.03 (m, 3H), 6.81 (d, J=8.4 Hz, 1H), 6.60-6.53 (m, 2H), 4.09 (s, 1H), 3.58 (d, J=12.9 Hz, 1H), 3.31-3.23 (m, 1H), 3.07-2.96 (m, 1H), 2.60 (d, J=13.0 Hz, 1H), 2.48-2.36 (m, 5H), 1.94-1.69 (m, 3H), 1.67-1.29 (m, 4H), 1.20-1.12 (m, 2H), 0.94 (s, 3H).

### Example #31

### (7aS,9R,11aS)-11a-Benzyl-9-hydroxy-9-propyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-hydroxy-9-propyl-6,7,7a,8,9,10,11,11a-octahydro-5H-dibenzo[a,c]cycloheptene-3-carboxylic acid (2-methyl-pyridin-3-yl)-amide (23, R²=Benzyl, R³=Propyl)

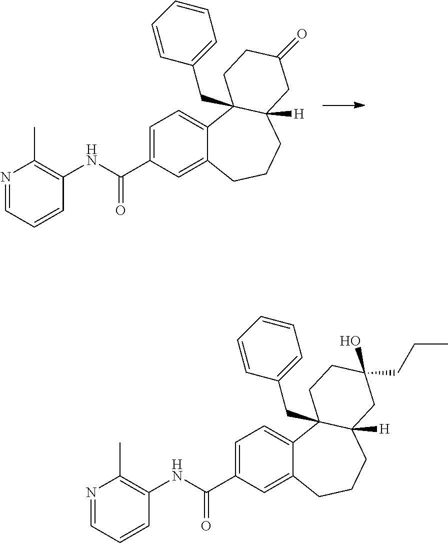

Compound 23 (R²=Benzyl, R³=Propyl) was prepared in a manner similar to the preparation of Compound 23 (R²=Benzyl, R³=Ethyl), substituting propylmagnesium bromide for ethylmagnesium bromide to yield (7aR,9S,11aR)-11a-benzyl-9-hydroxy-9-propyl-6,7,7a,8,9,10,11,11a-octahydro-5H-dibenzo[a,c]cycloheptene-3-carboxylic acid (2-methyl-pyridin-3-yl)-amide; compound with (7aS,9R,11aS)-11a-benzyl-9-hydroxy-9-propyl-6,7,7a,8,9,10,11,11a-octahydro-5H-dibenzo[a,c]cycloheptene-3-carboxylic acid (2-methyl-pyridin-3-yl)-amide (23, R²=Benzyl, R³=Propyl) (39 mg, 35%) as a white solid. LC/MS, method 2, Rₜ=2.47 min, MS m/z 483 (M+H)⁺. ¹H NMR (400 MHz, DMSO-d₆) δ 9.94 (s, 1H), 8.36-8.30 (m, 1H), 7.84-7.78 (m, 1H), 7.78-7.69 (m, 1H), 7.59-7.52 (m, 1H), 7.31-7.24 (m, 1H), 7.11-7.03 (m, 3H), 6.86-6.78 (m, 1H), 6.63-6.55 (m, 2H), 3.94 (s, 1H), 3.57 (d, J=12.8 Hz, 1H), 3.31-3.21 (m, 1H), 3.07-2.95 (m, 1H), 2.61 (d, J=13.1 Hz, 1H), 2.48-2.35 (m, 5H), 1.94-1.68 (m, 3H), 1.68-1.27 (m, 4H), 1.26-1.00 (m, 6H), 0.75 (t, J=7.0 Hz, 3H).

### Example #32

### (7aS,9S,11aS)-11a-Benzyl-9-ethynyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-11a-benzyl-9-ethynyl-9-hydroxy-6,7,7a,8,9,10,11,11a-octahydro-5H-dibenzo[a,c]cyclo heptene-3-carboxylic acid (2-methyl-pyridin-3-yl)-amide (24, R²=Benzyl, R³=Ethynyl)

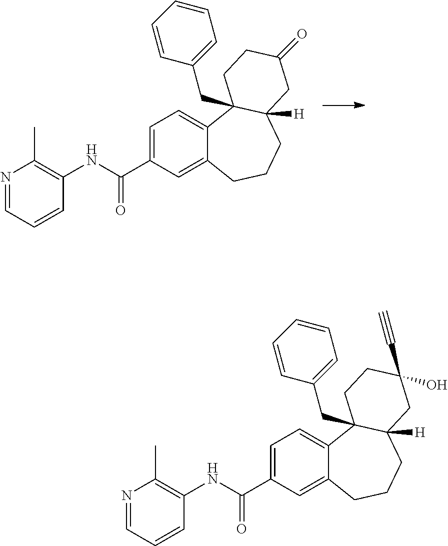

A solution of (7aS,11aS)-11a-benzyl-9-oxo-6,7,7a,8,9,10,11,11a-octahydro-5H-dibenzo[a,c]cycloheptene-3-carboxylic acid (2-methyl-pyridin-3-yl)-amide; compound with (7aR,11aR)-11a-benzyl-9-oxo-6,7,7a,8,9,10,11,11a-octahydro-5H-dibenzo[a,c]cycloheptene-3-carboxylic acid (2-methyl-pyridin-3-yl)-amide (22, R²=Benzyl) (150 mg, 0.342 mmol) in THF (6 mL) was cooled to about 0° C. and 3M lithium (trimethylsilyl)acetylide (6.84 mL, 3.42 mmol) in Et₂O was added dropwise. The reaction was stirred for about 30 min at about 0° C., then warmed to rt for about 1 h. The reaction was quenched with 10% aqueous AcOH (10 mL), extracted with EtOAc (2×25 mL), dried over Na₂SO₄, filtered and concentrated under reduced pressure. The residue was dissolved in THF (6 mL) and treated with TBAF (1M solution in THF, 0.342 mL, 0.342 mmol) for 1 h at rt. The reaction was diluted with water (10 mL) and extracted with EtOAc (2×20 mL). The combined extracts were dried over Na₂SO₄, filtered and concentrated under reduced pressure. The residue was purified on silica gel (12 g) using a gradient from 80-100% EtOAc in heptane. Product fractions were combined and concentrated to yield (7aR,9R,11aR)-11a-benzyl-9-ethynyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aS,9S,11aS)-11a-benzyl-9-ethynyl-9-hydroxy-6,7,7a,8,9,10,11,11a-octahydro-5H-dibenzo[a,c]cycloheptene-3-carboxylic acid (2-methyl-pyridin-3-yl)-amide (24, R²=Benzyl, R³=Ethynyl) (140 mg, 88%) as a white solid. LC/MS, method 2, Rₜ=2.30 min, MS m/z 465 (M+H)⁺, ¹H NMR (400 MHz, DMSO-d₆) δ 9.96 (s, 1H), 8.36-8.30 (m, 1H), 7.87-7.77 (m, 1H), 7.76-7.70 (m, 1H), 7.61-7.54 (m, 1H), 7.31-7.24 (m, 1H), 7.13-7.02 (m, 3H), 6.85-6.76 (m, 1H), 6.64-6.56 (m, 2H), 5.37 (s, 1H), 3.61 (d, J=12.9 Hz, 1H), 3.31-3.19 (m, 1H), 3.08-2.98 (m, 1H), 2.57 (d, J=13.1 Hz, 1H), 2.48-2.38 (m, 4H), 2.29-2.20 (m, 1H), 2.17-2.07 (m, 1H), 1.88-1.76 (m, 2H), 1.77-1.43 (m, 5H), 1.36-1.20 (m, 2H).

### Example #33

### (7aS,9R,11aS)-11a-Benzyl-9-ethoxymethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-ethoxymethyl-9-hydroxy-6,7,7a,8,9,10,11,11a-octahydro-5H-dibenzo[a,c]cycloheptene-3-carboxylic acid (2-methyl-pyridin-3-yl)-amide (29, R=Ethyl, R²=Benzyl)

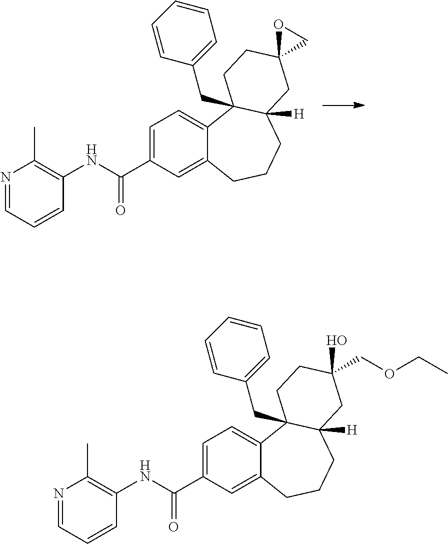

Compound 29 (R=Ethyl, R²=Benzyl) was prepared in a manner similar to the preparation of Compound 29 (R=Methyl, R²=Benzyl), substituting EtOH for MeOH to yield (7aS,9R,11aS)-11a-benzyl-9-ethoxymethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-ethoxymethyl-9-hydroxy-6,7,7a,8,9,10,11,11a-octahydro-5H-dibenzo[a,c]cycloheptene-3-carboxylic acid (2-methyl-pyridin-3-yl)-amide (29, R=Ethyl, R²=Benzyl) (48%) as a white solid. LC/MS, method 2, Rₜ=2.32 min, MS m/z 499 (M+H)⁺, ¹H NMR (400 MHz, DMSO-d₆) δ 9.95 (s, 1H), 8.33 (dd, J=4.7, 1.6 Hz, 1H), 7.81 (d, J=2.1 Hz, 1H), 7.74 (dd, J=8.0, 1.6 Hz, 1H), 7.59-7.53 (m, 1H), 7.27 (dd, J=8.0, 4.8 Hz, 1H), 7.09-7.03 (m, 3H), 6.82 (d, J=8.5 Hz, 1H), 6.61-6.55 (m, 2H), 4.14 (s, 1H), 3.58 (d, J=13.0 Hz, 1H), 3.25-3.34 (m, 3H), 3.08-2.92 (m, 3H), 2.64-2.56 (m, 1H), 2.48-2.40 (m, 5H), 1.95-1.86 (m, 1H), 1.86-1.72 (m, 2H), 1.68-1.45 (m, 3H), 1.43-1.35 (m, 1H), 1.28-1.20 (m, 1H), 1.16-1.08 (m, 1H), 0.97 (t, J=8.0 Hz, 3H).

### Example #34 and #35

### (7aS,9R,11aR)-9-Benzyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide; compound with (7aR,9S,11aS)-9-benzyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide (23, R²=Propyl, R³=Benzyl) and (7aS,9S,11aR)-9-benzyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide; compound with (7aR,9R,11aS)-9-benzyl-9-hydroxy-11a-propyl-6,7,7a,8,9,10,11,11a-octahydro-5H-dibenzo[a,c]cycloheptene-3-carboxylic acid (2-methyl-pyridin-3-yl-amide (24. R²=Pronyl. R³=Benzyl)

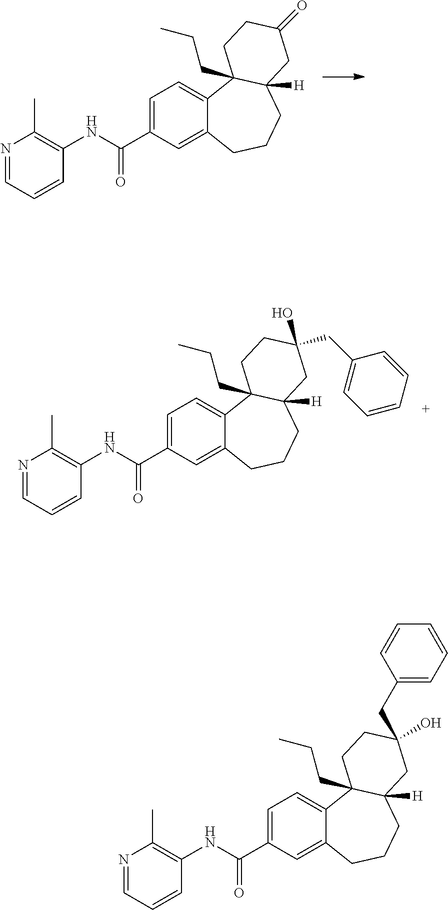

The compounds 23 (R²=Propyl, R³=Benzyl) and 24 (R²=Propyl, R³=Benzyl) were prepared in a manner similar to the preparation of compounds 23 (R²=Propyl, R³=Ethyl) and 24 (R²=Propyl, R³=Ethyl) substituting benzylmagnesiumchloride for ethylmagnesium bromide to yield (7aS,9R,11aR)-9-benzyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide; compound with (7aR,9S,11aS)-9-benzyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide (23, R²=Propyl, R³=Benzyl) (7%) as a white solid, LC/MS, method 2, Rₜ=2.67 min, MS m/z 481 (M−H)⁻. ¹H NMR (400 MHz, DMSO-d₆) δ 9.93 (s, 1H), 8.32 (dd, J=4.7, 1.5 Hz, 1H), 7.75-7.69 (m, 3H), 7.35 (d, J=8.3 Hz, 1H), 7.25 (dt, J=14.7, 7.4 Hz, 1H), 7.20-7.14 (m, 2H), 7.15-7.09 (m, 3H), 4.09 (s, 1H), 3.05-2.96 (m, 1H), 2.92-2.80 (m, 1H), 2.47-2.42 (m, 4H), 2.32-2.19 (m, 3H), 2.03-1.94 (m, 1H), 1.75-1.11 (m, 10H), 1.10-1.03 (m, 1H), 0.73 (t, J=6.9 Hz, 3H), 0.72-0.61 (m, 1H) and (7aS,9S,11aR)-9-benzyl-9-hydroxy-11a-propyl-6,7,7a,8,9,10,11,11a-octahydro-5H dibenzo[a,c]cycloheptene-3-carboxylic acid (2-methyl-pyridin-3-yl)-amide; compound with (7aR,9R,11aS)-9-benzyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide (24, R²=Propyl, R³=Benzyl) (38%) as a white solid, LC/MS, method 2, Rₜ=2.95 min, MS m/z 481 (M−H)⁻=481, ¹H NMR (400 MHz, DMSO-d₆) δ 9.92 (s, 1H), 8.31 (dd, J=4.7, 1.6 Hz, 1H), 7.76-7.65 (m, 3H), 7.39 (d, J=8.4 Hz, 1H), 7.28-7.10 (m, 6H), 4.09 (s, 1H), 3.04-2.93 (m, 2H), 2.90-2.82 (m, 1H), 2.73-2.65 (m, 1H), 2.54-2.49 (m, 1H), 2.41 (s, 3H), 2.26-2.17 (m, 1H), 2.10-2.01 (m, 2H), 1.73-1.24 (m, 8H), 1.17-1.08 (m, 1H), 1.04-0.94 (m, 1H), 0.81-0.62 (m, 4H).

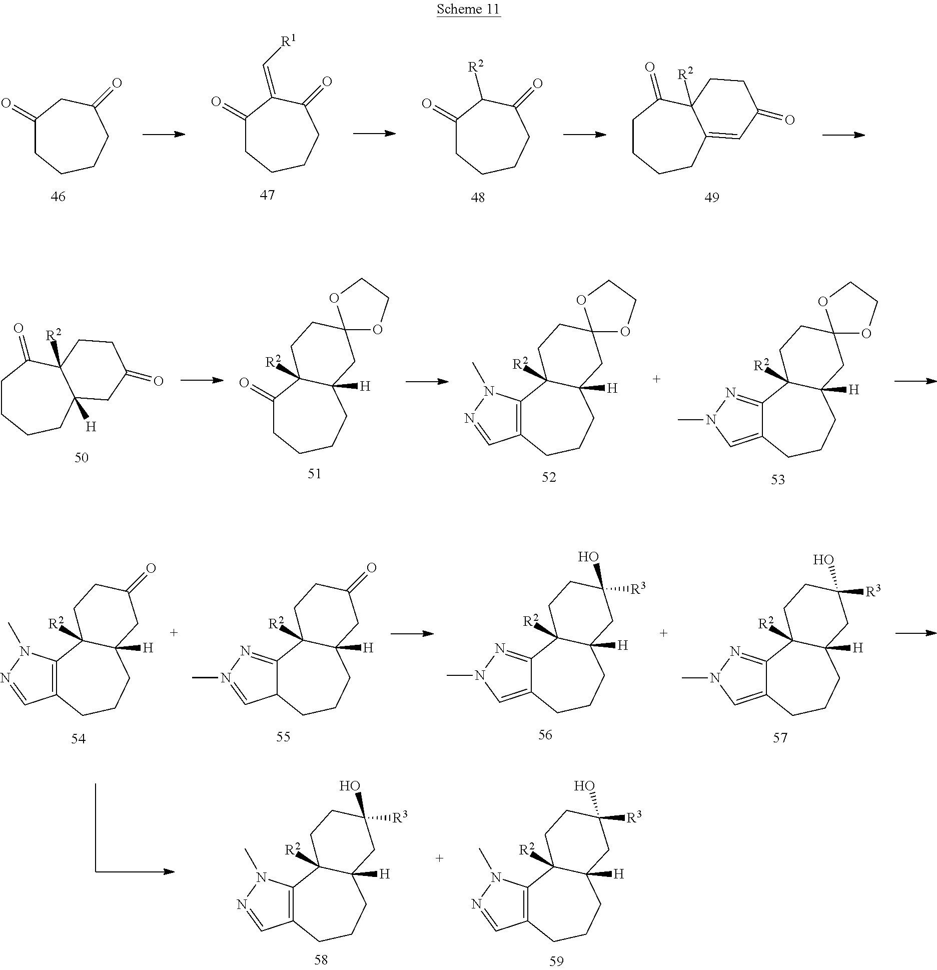

### Example #36

### (4aS,9aS)-4a-Benzyl-octahydro-benzocycloheptene-2,5-dione; compound with (4aR,9aR)-4a-benzyl-octahydro-benzocycloheptene-2,5-dione (50, R²=Benzyl)

### Step 1: 2-Benzylidene-cycloheptane-1,3-dione (47, R¹=Phenyl)

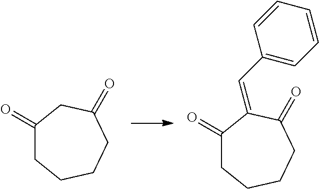

A mixture of benzaldehyde (28.4 mL, 281 mmol) and (S)-pyrrolidine-2-carboxylic acid (0.463 g, 4.02 mmol) was stirred neat at rt and cycloheptane-1,3-dione (46) (5.07 g, 40.2 mmol) was added dropwise over about 30 min The mixture was stirred for about 4 h at rt and then purified on silica gel (330 g) using a gradient from 10 to 30% EtOAc in heptane. The product fractions were combined and concentrated to a pale yellow oil that solidified on continued drying to yield 2-benzylidene-cycloheptane-1,3-dione (47, R¹=Phenyl) (5.90 g, 68%) as an off-white solid, LC/MS, method 1, Rₜ=0.66 min, MS m/z 215 (M+H)⁺, ¹H NMR (400 MHz, DMSO-d₆) δ 7.52-7.43 (m, 3H), 7.43-7.34 (m, 3H), 2.84-2.77 (m, 2H), 2.55-2.49 (m, 2H), 1.96-1.83 (m, 4H).

### Step 2: 2-Benzyl-cycloheptane-1,3-dione (48, R²=Benzyl)

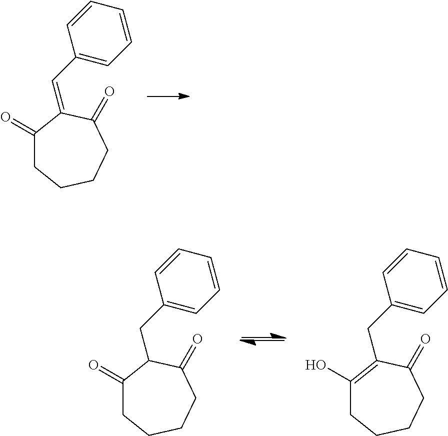

A solution containing 2-benzylidene-cycloheptane-1,3-dione (47, R¹=Phenyl) (5.89 g, 27.5 mmol) in toluene (50 mL) was treated with 20% Pd(OH)₂ on carbon (0.965 g) and the mixture was shaken under about 50 psi of hydrogen for about 1 h. The solution was filtered through a pad of Celite®, rinsed with toluene, concentrated under reduced pressure to a clear oil and dried to constant weight to yield 2-benzyl-cycloheptane-1,3-dione (48, R²=Benzyl) (5.95 g, 100%), LC/MS, method 1, Rₜ=1.48 min, MS m/z 217 (M+H)⁺, ¹H NMR indicated that the product exists as a mixture of keto and enol forms (about 3:1).

### Step #3: 4a-Benzyl-4,4a,6,7,8,9-hexahydro-3H-benzocycloheptene-2,5-dione (49, R²=Benzyl)

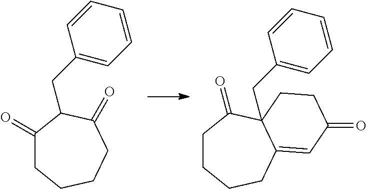

A mixture of 2-benzylcycloheptane-1,3-dione (48, R²=Benzyl) (5.40 g, 25.0 mmol) and but-3-en-2-one (3.07 mL, 37.5 mmol) was treated with TEA (0.174 mL, 1.25 mmol) and the mixture was stoppered and stirred at about 50° C. for about 5 days. The mixture was dried under reduced pressure. The residue was dissolved in toluene (100 mL), pyridine (2.07 mL, 25.0 mmol) and acetic acid (1.43 mL, 25.0 mmol) were added and the mixture was stirred at rt for about 1 h, then at about 50° C. for about 5 h. The reaction was cooled to rt and stirred about 18 h. Water (25 mL) was added and the mixture was stirred about 1 h. The layers were separated and the organic layer was dried over Na₂SO₄, filtered and concentrated under reduced pressure. The residue was purified on silica gel (120 g) using a gradient from 10% to 30% EtOAc in heptane. The product fractions were combined and concentrated under reduced pressure to about 70 mL. The product precipitated and was collected by filtration, washed with heptane (25 mL) and dried under reduced pressure to yield 4a-benzyl-4,4a,6,7,8,9-hexahydro-3H-benzocycloheptene-2,5-dione (49, R²=Benzyl) as an off-white powder (5.33 g, 80%), LC/MS, method 1, Rₜ=0.72 min, MS m/z 269 (M+H)⁺, ¹H NMR (400 MHz, DMSO-d₆) δ 7.26-7.16 (m, 3H), 7.16-7.10 (m, 2H), 6.17 (s, 1H), 3.24-3.11 (m, 2H), 2.79-2.69 (m, 1H), 2.39-2.30 (m, 1H), 2.30-2.14 (m, 2H), 2.10-1.98 (m, 1H), 1.95-1.75 (m, 4H), 1.48-1.26 (m, 3H).

### Step #4: (4aS,9aS)-4a-Benzyl-octahydro-benzocycloheptene-2,5-dione; compound with (4aR,9aR)-4a-benzyl-octahydro-benzocycloheptene-2,5-dione (50, R²=Benzyl)

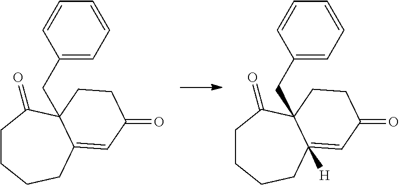

A solution of 4a-benzyl-4,4a,6,7,8,9-hexahydro-3H-benzocycloheptene-2,5-dione (49, R²=Benzyl) (4.80 g, 17.89 mmol) containing 20% Pd(OH)₂ on carbon (1.90 g) was dissolved in toluene (89 mL). The reaction was shaken under hydrogen (50 psi) for about 18 h. The reaction was filtered through a pad of Celite® and concentrated to dryness to yield (4aS,9aS)-4a-benzyl-octahydro-benzocycloheptene-2,5-dione; compound with (4aR,9aR)-4a-benzyl-octahydro-benzocycloheptene-2,5-dione (50, R²=Benzyl) (5.04 g, 99%) as a white solid, LC/MS, method 1, Rₜ=0.74 min, MS m/z 269 (M−H)⁻, ¹H NMR (400 MHz, DMSO-d₆) δ 7.29-7.19 (m, 3H), 7.08-7.03 (m, 2H), 3.28-3.16 (m, 2H), 3.13-3.04 (m, 1H), 2.85-2.80 (m, 1H), 2.71-2.60 (m, 1H), 2.17-2.00 (m, 3H), 1.88-1.62 (m, 5H), 1.57-1.07 (m, 4H).

### Example #37

### (+/−) Compound 51 (R²=Benzyl)

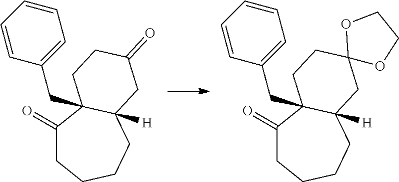

To solution of (4aS,9aS)-4a-benzyl-octahydro-benzocycloheptene-2,5-dione; compound with (4aR,9aR)-4a-benzyl-octahydro-benzocycloheptene-2,5-dione (50, R²=Benzyl) (5.04 g, 17.7 mmol) in toluene (136 mL) was added ethylene glycol (1.98 mL, 35 mmol), and toluene-4-sulfonic acid hydrate (0.337 g, 1.77 mmol). The reaction was stirred at reflux for about 3 h, removing water using a Dean-Stark trap. The reaction was cooled to rt, washed with saturated aqueous NaHCO₃ (100 mL), dried over Na₂SO₄, filtered and concentrated to an oil. The residue was purified on silica gel (120 g) using a gradient 0-40% EtOAc in heptane. The product fractions were combined and concentrated to yield Compound 51 (R²=Benzyl) (4.10 g, 74%) as a white solid, LC/MS, method 2, Rₜ=2.57 min, MS m/z 315 (M+H)⁺, ¹H NMR (400 MHz, DMSO-d₆) δ 7.28-7.10 (m, 3H), 6.98-6.93 (m, 2H), 3.88-3.76 (m, 4H), 3.07-2.89 (m, 2H), 2.20-1.95 (m, 3H), 1.93-1.58 (m, 6H), 1.58-1.16 (m, 6H).

### Examples #38 and #39

### (6aS,8R,10aS)-10a-Benzyl-8-ethyl-1-methyl-1,4,5,6,6a,7,8,9,10,10a-decahydro-1,2-diaza-benzo[e]azulen-8-ol; compound with (6aR,8S,10aR)-10a-benzyl-8-ethyl-1-methyl-1,4,5,6,6a,7,8,9,10,10a-decahydro-1,2-diaza-benzo[e]azulen-8-ol (58, R²=Benzyl, R³=Ethyl) and (6aS,8S,10aS)-10a-Benzyl-8-ethyl-1-methyl-1,4,5,6,6a,7,8,9,10,10a-decahydro-1,2-diaza-benzo[e]azulen-8-ol; compound with (6aR,8R,10aR)-10a-benzyl-8-ethyl-1-methyl-1,4,5,6,6a,7,8,9,10,10a-decahydro-1,2-diaza-benzo[e]azulen-8-ol (59, R²=Benzyl, R³=Ethyl)

### Step 1: (+/−) Compound 52 (R²=Benzyl) and (+/−) Compound 53 (R²=Benzyl)

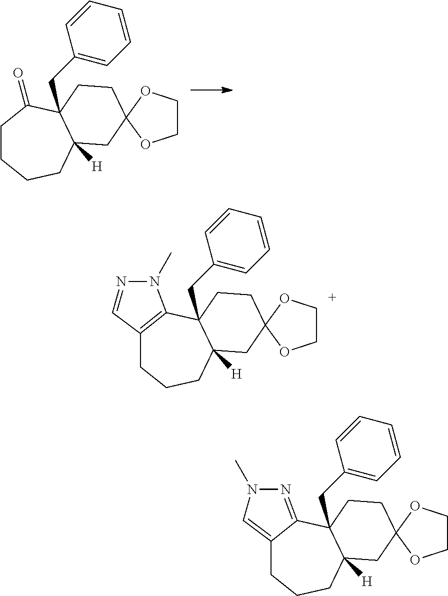

A mixture of (4aS,9aS)-4a-benzyloctahydrospiro[benzo[7]annulene-2,2′-[1,3]dioxolan]-5(1H)-one; compound with (4aR,9aR)-4a-benzyloctahydrospiro[benzo[7]annulene-2,2′-[1,3]dioxolan]-5(1H)-one (51, R²=Benzyl) (0.500 g, 1.59 mmol) was treated with 1-tert-butoxy-N,N,N′,N′-tetramethylmethanediamine (1.39 g, 7.95 mmol). The flask was fitted with an air condenser then heated at about 150° C. for about 3 h. The mixture was cooled to rt then 1-tert-butoxy-N,N,N′,N′-tetramethylmethanediamine (0.831 g, 4.77 mmol) was added. The mixture was heated at about 150° C. for about 2 h. The mixture was cooled to rt. The solvents were removed under reduced pressure, the material was triturated with heptane (˜8 mL) then the mixture was concentrated to dryness under reduced pressure. The material was treated with EtOH (8 mL) and methylhydrazine (0.513 g, 11.1 mmol). The mixture was warmed to about 60° C. for about 1 h then to reflux for about 3 h. The mixture was cooled to rt then the solvents were removed under reduced pressure. The material was treated with water (20 mL) then extracted with DCM (2×20 mL). The combined organics were extracted with water then dried over anhydrous MgSO₄, filtered and the filtrate concentrated under reduced pressure. The material was dissolved in toluene (30 mL) then treated with p-toluenesulfonic acid monohydrate (0.015 g, 0.072 mmol). The flask was fitted with a Dean-Stark apparatus then the mixture was heated to reflux for about 30 min. The mixture was cooled then concentrated under reduced pressure. The material was purified on silica gel (12 g) eluting with a gradient of 10-75% EtOAc in heptane. Evaporation of the appropriate fractions gave (+/−) Compound 52 (R²=Benzyl) (0.106 g, 19%). LC/MS, method 2, Rₜ=2.69 min, MS m/z 383 (M+H)⁺; ¹H NMR (400 MHz, DMSO-d₆) δ 7.21-7.05 (m, 4H), 6.65 (d, J=6.5 Hz, 2H), 3.92-3.78 (m, 4H), 3.33 (d, J=14.6 Hz, 1H), 2.87 (s, 3H), 2.79-2.68 (m, 1H), 2.66-2.52 (m, 1H), 2.47 (d, J=14.6 Hz, 1H), 2.39-2.25 (m, 2H), 2.10-2.01 (m, 1H), 1.77-1.54 (m, 5H), 1.54-1.36 (m, 2H), 1.23-1.12 (m, 1H) and (+/−) Compound 53 (R²=Benzyl), (0.207 g, 37%); LC/MS, method 2, Rₜ=2.59 min, MS m/z 383(M+H)⁺. ¹H NMR (400 MHz, DMSO-d₆) δ 7.36 (s, 1H), 7.13-7.07 (m, 3H), 6.70-6.63 (m, 2H), 3.91-3.69 (m, 4H), 3.62 (s, 3H), 3.11 (d, J=12.8 Hz, 1H), 2.76-2.66 (m, 1H), 2.66-2.52 (m, 1H), 2.40-2.29 (m, 1H), 1.98-1.93 (m, 1H), 1.87-1.77 (m, 1H), 1.73-1.63 (m, 3H), 1.47-1.22 (m, 6H).

### Step 2: (6aS,10aS)-10a-Benzyl-1-methyl-4,5,6,6a,7,9,10,10a-octahydro-1H-1,2-diaza-benzo[e]azulen-8-one compound; with (6aR,10aR)-10a-benzyl-1-methyl-4,5,6,6a,7,9,10,10a-octahydro-1H-1,2-diaza-benzo[e]azulen-8-one (54, R²=Benzyl)

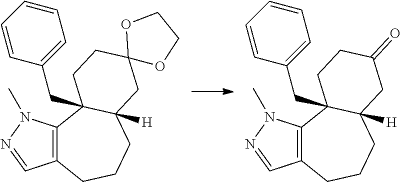

Compound 52 (R²=Benzyl) (0.200 g, 0.567 mmol) was dissolved in acetone (6 mL) then treated with 37 wt % hydrochloric acid (0.070 mL, 0.84 mmol). The mixture was stirred for about 14 h at rt. The solvents were removed under reduced pressure then the material was dissolved in acetone (6 mL) and treated with 37 wt % hydrochloric acid (0.070 mL, 0.84 mmol). The mixture was stirred at rt for about 1 h then the solvents were removed by evaporation under reduced pressure. The material was treated with water (20 mL) then DCM (20 mL). The mixture was basified with 50 wt % aqueous NaOH then the layers were separated. The aqueous layer was extracted with DCM (15 mL) then the combined organics were dried over anhydrous MgSO₄, filtered and the filtrate concentrated under reduced pressure to give (6aS,10aS)-10a-benzyl-1-methyl-4,5,6,6a,7,9,10,10a-octahydro-1H-1,2-diaza-benzo[e]azulen-8-one compound; with (6aR,10aR)-10a-benzyl-1-methyl-4,5,6,6a,7,9,10,10a-octahydro-1H-1,2-diaza-benzo[e]azulen-8-one (54, R²=Benzyl) (0.170 g, 97%); LC/MS, method 3, Rₜ=2.31 min, MS m/z 309 (M+H)⁺.

### Step#3: (6aS,8R,10aS)-10a-Benzyl-8-ethyl-1-methyl-1,4,5,6,6a,7,8,9,10,10a-decahydro-1,2-diaza-benzo[e]azulen-8-ol; compound with (6aR,8S,10aR)-10a-benzyl-8-ethyl-1-methyl-1,4,5,6,6a,7,8,9,10,10a-decahydro-1,2-diaza-benzo[e]azulen-8-ol (59, R²=Benzyl, R³=Ethyl) and (6aS,8S,10aS)-10a-benzyl-8-ethyl-1-methyl-1,4,5,6,6a,7,8,9,10,10a-decahydro-1,2-diaza-benzo[e]azulen-8-ol; compound with (6aR,8R,10aR)-10a-benzyl-8-ethyl-1-methyl-1,4,5,6,6a,7,8,9,10,10a-decahydro-1,2-diaza-benzo[e]azulen-8-ol (58, R²=Benzyl, R³=Ethyl)

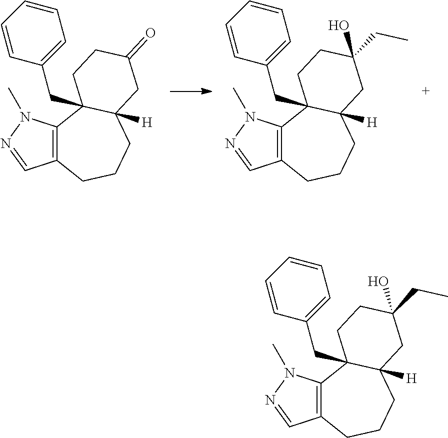

A 3 necked round bottom flask equipped with thermometer, septum and nitrogen line was charged with THF (3 mL). The solvent was cooled to about 0° C. then ethylmagnesium bromide (3M solution in Et₂O, 1.47 mL, 4.41 mmol) was added slowly. (6aS,10aS)-10a-benzyl-1-methyl-4,5,6,6a,7,9,10,10a-octahydro-1H-1,2-diaza-benzo[e]azulen-8-one compound; with (6aR,10aR)-10a-benzyl-1-methyl-4,5,6,6a,7,9,10,10a-octahydro-1H-1,2-diaza-benzo[e]azulen-8-one (54, R²=Benzyl) (0.170 g, 0.551 mmol) dissolved in THF (3 mL) was added to the Grignard reagent mixture maintaining the internal temperature≦5° C. After about 5 min the mixture was treated with acetic acid (0.32 mL, 5.5 mmol) keeping the internal temperature<5° C. The mixture was then diluted with water (20 mL) and extracted with DCM (2×20 mL). The combined organics were dried over MgSO₄, then filtered and the filtrate concentrated under reduced pressure. The material was purified on a silica gel column (10 g) eluting with EtOAc. Two major products were isolated. The higher R_{f} material was further purified on a silica gel column (10 g) with 50% EtOAc in heptane as eluent to give (6aS,8R,10aS)-10a-benzyl-8-ethyl-1-methyl-1,4,5,6,6a,7,8,9,10,10a-decahydro-1,2-diaza-benzo[e]azulen-8-ol; compound with (6aR,8S,10aR)-10a-benzyl-8-ethyl-1-methyl-1,4,5,6,6a,7,8,9,10,10a-decahydro-1,2-diaza-benzo[e]azulen-8-ol (58, R²=Benzyl, R³=Ethyl) (0.092 g. 49%); LC/MS, method 2, Rₜ=2.49 min, MS m/z 339 (M+H)⁺; ¹H NMR (400 MHz, DMSO-d₆) δ 7.20-7.12 (m, 3H), 7.09 (s, 1H), 6.67-6.60 (m, 2H), 3.94 (s, 1H), 3.35 (d, J=12.8 Hz, 1H), 2.84 (s, 3H), 2.78-2.69 (m, 1H), 2.63-2.53 (m, 1H), 2.45 (d, J=12.8 Hz, 1H), 2.39-2.28 (m, 1H), 2.28-2.20 (m, 1H), 2.14-2.05 (m, 1H), 1.84-1.72 (m, 1H), 1.67-1.45 (m, 4H), 1.39-1.29 (m, 1H), 1.27-1.17 (m, 3H), 0.99-0.89 (m, 1H), 0.75 (t, J=7.5 Hz, 3H). The lower R_{f} material was further purified on a 5 g silica column with EtOAc as an eluent to give (6aS,8S,10aS)-10a-benzyl-8-ethyl-1-methyl-1,4,5,6,6a,7,8,9,10,10a-decahydro-1,2-diaza-benzo[e]azulen-8-ol; compound with (6aR,8R,10aR)-10a-benzyl-8-ethyl-1-methyl-1,4,5,6,6a,7,8,9,10,10a-decahydro-1,2-diaza-benzo[e]azulen-8-ol (59, R²=Benzyl, R³=Ethyl) (0.028 g, 15%); LC/MS, method 2, Rₜ=2.60 min, MS m/z 339 (M+H)⁺.

### Examples #40 and #41

### (6aS,8R,10aS)-10a-Benzyl-8-ethyl-2-methyl-2,4,5,6,6a,7,8,9,10,10a-decahydro-1,2-diaza-benzo[e]azulen-8-ol; compound with (6aR,8S,10aR)-10a-benzyl-8-ethyl-2-methyl-2,4,5,6,6a,7,8,9,10,10a-decahydro-1,2-diaza-benzo[e]azulen-8-ol (56, R²=Benzyl, R³=Ethyl) and: (6aS,8S,10aS)-10a-benzyl-8-ethyl-2-methyl-2,4,5,6,6a,7,8,9,10,10a-decahydro-1,2-diaza-benzo[e]azulen-8-ol; compound with (6aR,8R,10aR)-10a-benzyl-8-ethyl-2-methyl-2,4,5,6,6a,7,8,9,10,10a-decahydro-1,2-diaza-benzo[e]azulen-8-ol (57, R²=Benzyl, R³=Ethyl)

### Step 1: (6aS,10aS)-10a-Benzyl-2-methyl-2,5,6,6a,7,9,10,10a-octahydro-4H-1,2-diaza-benzo[e]azulen-8-one compound; with (6aR,10aR)-10a-benzyl-2-methyl-2,5,6,6a,7,9,10,10a-octahydro-4H-1,2-diaza-benzo[e]azulen-8-one (55, R²=Benzyl)

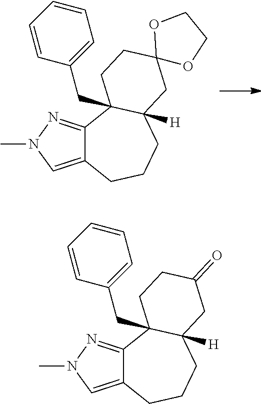

(+/−) Compound 53 (R²=Benzyl) (0.150 g, 0.426 mmol) in acetone (7 mL) was treated with 37 wt % hydrochloric acid (0.083 mL, 1.0 mmol) then stirred at rt for about 16 h. The mixture was concentrated under reduced pressure then partitioned between DCM (20 mL) and saturated aqueous NaHCO₃ (10 mL). The layers were separated then the aqueous layer was extracted with DCM (15 mL). The combined organics were dried over anhydrous MgSO₄ and filtered then the filtrate was concentrated under reduced pressure to give (6aS,10aS)-10a-benzyl-2-methyl-2,5,6,6a,7,9,10,10a-octahydro-4H-1,2-diaza-benzo[e]azulen-8-one compound; with (6aR,10aR)-10a-benzyl-2-methyl-2,5,6,6a,7,9,10,10a-octahydro-4H-1,2-diaza-benzo[e]azulen-8-one (55, R²=Benzyl) (0.129 g, 98%); LC/MS, method 3, Rₜ=2.50 min, MS m/z 309 (M+H)⁺.

### Step 2: (6aS,8R,10aS)-10a-Benzyl-8-ethyl-2-methyl-2,4,5,6,6a,7,8,9,10,10a-decahydro-1,2-diaza-benzo[e]azulen-8-ol; compound with (6aR,8S,10aR)-10a-benzyl-8-ethyl-2-methyl-2,4,5,6,6a,7,8,9,10,10a-decahydro-1,2-diaza-benzo[e]azulen-8-ol (56, R²=Benzyl, R³=Ethyl) and (6aS,8S,10aS)-10a-benzyl-8-ethyl-2-methyl-2,4,5,6,6a,7,8,9,10,10a-decahydro-1,2-diaza-benzo[e]azulen-8-ol; compound with (6aR,8R,10aR)-10a-benzyl-8-ethyl-2-methyl-2,4,5,6,6a,7,8,9,10,10a-decahydro-1,2-diaza-benzo[e]azulen-8-ol (57, R²=Benzyl, R³=Ethyl)

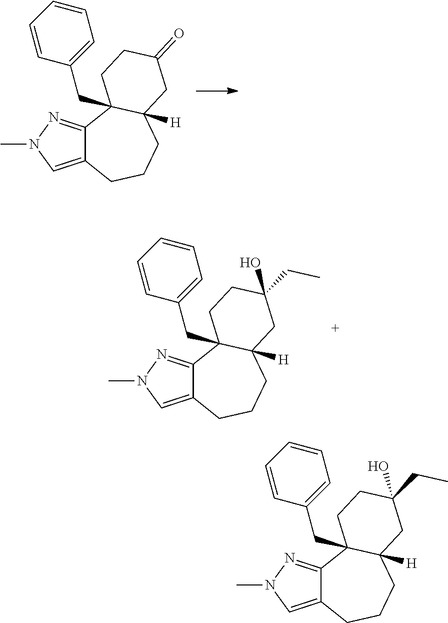

A 25 mL 3 necked round bottom flask equipped with a nitrogen line, thermometer and septum was charged with THF (3 mL). The mixture was cooled to about 0° C. then ethylmagnesium bromide (3M solution in Et₂O, 1.1 mL, 3.3 mmol) was added. The mixture was cooled to about 0° C. then the (6aS,10aS)-10a-benzyl-2-methyl-2,5,6,6a,7,9,10,10a-octahydro-4H-1,2-diaza-benzo[e]azulen-8-one: compound with (6aR,10aR)-10a-benzyl-2-methyl-2,5,6,6a,7,9,10,10a-octahydro-4H-1,2-diaza-benzo[e]azulen-8-one (55, R²=Benzyl) (0.129 g, 0.418 mmol) in THF (3 mL) was added keeping the internal temp<5° C. The mixture was stirred at about 0° C. for about 15 min, then the reaction was treated with acetic acid (0.24 mL, 4.2 mmol). The mixture was added to water (25 mL) then extracted with DCM (20 mL then 15 mL). The combined organics were dried over anhydrous MgSO₄, then filtered and the filtrate was concentrated under reduced pressure. The material was purified on a silica gel column (10 g) using EtOAc as eluent. Two major products were isolated. The higher R_{f} material was further purified on a silica gel column (10 g) using 40% EtOAc in heptane as eluent to give (6aS,8R,10aS)-10a-benzyl-8-ethyl-2-methyl-2,4,5,6,6a,7,8,9,10,10a-decahydro-1,2-diaza-benzo[e]azulen-8-ol; compound with (6aR,8S,10aR)-10a-benzyl-8-ethyl-2-methyl-2,4,5,6,6a,7,8,9,10,10a-decahydro-1,2-diaza-benzo[e]azulen-8-ol (56, R²=Benzyl, R³=Ethyl) (0.043 g, 31%); LC/MS, method 2, Rₜ=2.63 min, MS m/z 339 (M+H)⁺. ¹H NMR (400 MHz, DMSO-d₆) δ 7.30 (s, 1H), 7.10-7.05 (m, 3H), 6.65-6.59 (m, 2H), 3.61 (s, 1H), 3.56 (s, 3H), 3.12 (d, J=12.8 Hz, 1H), 2.73-2.67 (m, 1H), 2.54-2.50 (m, 1H), 2.40-2.32 (m, 1H), 2.20-2.14 (m 1H), 1.82-1.76 (m, 1H), 1.69-1.04 (m, 11H), 0.70 (t, J=7.5 Hz, 3H). The lower R_{f} material was further purified on a silica gel column (10 g) using EtOAc as eluent to give (6aS,8S,10aS)-10a-benzyl-8-ethyl-2-methyl-2,4,5,6,6a,7,8,9,10,10a-decahydro-1,2-diaza-benzo[e]azulen-8-ol; compound with (6aR,8R,10aR)-10a-benzyl-8-ethyl-2-methyl-2,4,5,6,6a,7,8,9,10,10a-decahydro-1,2-diaza-benzo[e]azulen-8-ol (57, R²=Benzyl, R³=Ethyl) (0.0095 g, 7%); LC/MS, method 2, Rₜ=2.71 min, MS m/z 339 (M+H)⁺. ¹H NMR (400 MHz, DMSO-d₆) δ 7.33 (s, 1H), 7.08-7.05 (m, 3H), 6.63-6.56 (m, 2H), 3.76 (s, 1H), 3.58 (s, 3H), 3.09 (d, J=12.8 Hz, 1H), 2.75-2.65 (m, 1H), 2.47 (d, J=12.8 Hz, 1H), 2.37-2.24 (m, 1H), 1.98-1.89 (m, 1H), 1.72-1.60 (m, 4H), 1.52-1.09 (m, 8H), 0.74 (t, J=7.4 Hz, 3H).

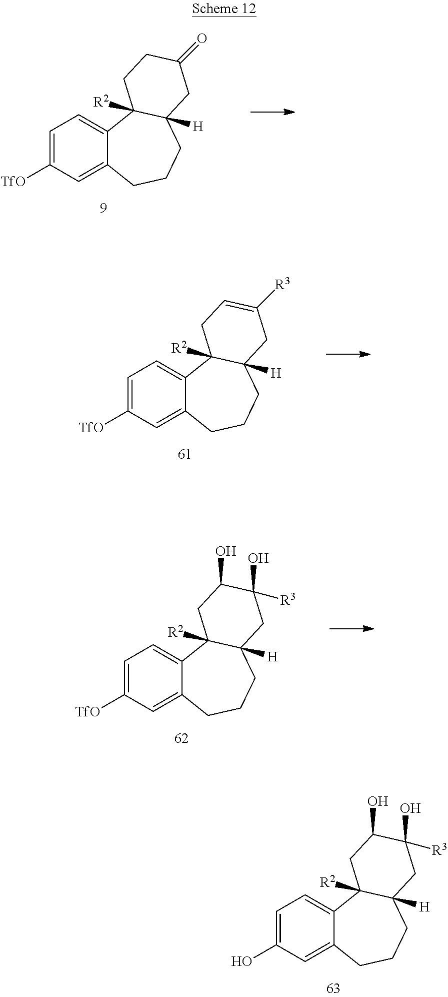

### Example #42

### (2R,3R,4aS,11bR)-11b-Benzyl-3-phenyl-2,3,4,4a,5,6,7,11b-octahydro-1H-dibenzo[a,c]cycloheptene-2,3,9-triol compound with (2S,3S,4aR,11bS)-11b-benzyl-3-phenyl-2,3,4,4a,5,6,7,11b-octahydro-1H-dibenzo[a,c]cycloheptene-2,3,9-triol (63, R²=Benzyl, R³=Phenyl)

### Step 1: Trifluoro-methanesulfonic acid (7aS,11aS)-11a-benzyl-9-phenyl-6,7,7a,8,11,11a-hexahydro-5H-dibenzo[a,c]cyclohepten-3-yl ester compound with trifluoro-methanesulfonic acid (7aR,11aR)-11a-benzyl-9-phenyl-6,7,7a,8,11,11a-hexahydro-5H-dibenzo[a,c]cyclohepten-3-yl ester (61, R²=Benzyl, R³=Phenyl)

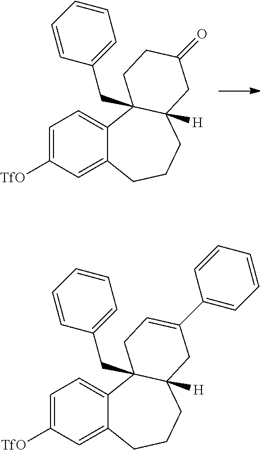

Phenylmagnesium bromide (1M solution in THF, 9.72 mL, 9.72 mmol) in THF (15 mL) was cooled to about 0° C. To the solution was added trifluoro-methanesulfonic acid (7aS,11aS)-11a-benzyl-9-oxo-6,7,7a,8,9,10,11,11a-octahydro-5H-dibenzo[a,c]cyclohepten-3-yl ester compound with trifluoro-methanesulfonic acid (7aR,11aR)-11a-benzyl-9-oxo-6,7,7a,8,9,10,11,11a-octahydro-5H-dibenzo[a,c]cyclohepten-3-yl ester (9, R²=Benzyl) (1.10 g, 2.43 mmol). The mixture stirred for about 1 h and then saturated aqueous NH₄Cl (15 mL) was added and the organics were concentrated in vacuo, extracted with EtOAc (2×30 mL), dried over MgSO₄ and concentrated to provide trifluoro-methanesulfonic acid (7aS,11aS)-11a-benzyl-9-hydroxy-9-phenyl-6,7,7a,8,9,10,11,11a-octahydro-5H-dibenzo[a,c]cyclohepten-3-yl ester compound with trifluoro-methanesulfonic acid (7aR,11aR)-11a-benzyl-9-hydroxy-9-phenyl-6,7,7a,8,9,10,11,11a-octahydro-5H-dibenzo[a,c]cyclohepten-3-yl ester (1.13 g, 88%) as a pale yellow oil. LC/MS, method 3, Rₜ=3.26 min, MS m/z 589 (M+OAc)⁻. The resulting oil was dissolved in toluene (20 mL) and then potassium hydrogen sulfate (0.142 mL, 2.34 mmol) was added and the mixture was heated to reflux for about 17 h. The residue was concentrated to dryness and then purified on silica gel (40 g) eluting with 0 to 20% EtOAc in heptane to provide trifluoro-methanesulfonic acid (7aS,11aS)-11a-benzyl-9-phenyl-6,7,7a,8,11,11a-hexahydro-5H-dibenzo[a,c]cyclohepten-3-yl ester compound with trifluoro-methanesulfonic acid (7aR,11aR)-11a-benzyl-9-phenyl-6,7,7a,8,11,11a-hexahydro-5H-dibenzo[a,c]cyclohepten-3-yl ester (61, R²=Benzyl, R³=Phenyl) (1.17 g, 108%, contained ˜8% solvent) as a white solid. LC/MS, method 4, Rₜ=3.19 min, MS m/z 571 (M+OAc)⁻. ¹H NMR (400 MHz, DMSO-d₆) δ 7.33 (d, J=2.9 Hz, 1H), 7.20-7.27 (m, 4H), 7.13-7.18 (m, 1H), 7.00-7.08 (m, 3H), 6.96 (dd, J=8.8, 2.9 Hz, 1H), 6.76 (d, J=8.9 Hz, 1H), 6.56-6.51 (m, 2H), 6.19 (bs, 1H), 3.91 (d, J=13.2 Hz, 1H), 3.45-3.41 (m, 1H), 3.06-2.96 (m, 1H), 2.64-2.57 (m, 1H), 2.43-2.35 (m, 2H), 2.35-2.26 (m, 1H), 2.24-2.15 (m, 1H), 2.07-1.93 (m, 1H), 1.91-1.81 (m, 2H), 1.91-1.68 (m, 1H), 1.63-1.47 (m, 1H).

### Step 2: Trifluoromethanesulfonic acid (7aS,9R,10R,11aR)-11a-benzyl-9,10-dihydroxy-9-phenyl-6,7,7a,8,9,10,11,11a-octahydro-5H-dibenzo[a,c]cyclohepten-3-yl ester compound with trifluoromethanesulfonic acid (7aR,9S,10S,11aS)-11a-benzyl-9,10-dihydroxy-9-phenyl-6,7,7a,8,9,10,11,11a-octahydro-5H-dibenzo[a,c]cyclohepten-3-yl ester (62, R²=Benzyl, R³=Phenyl)

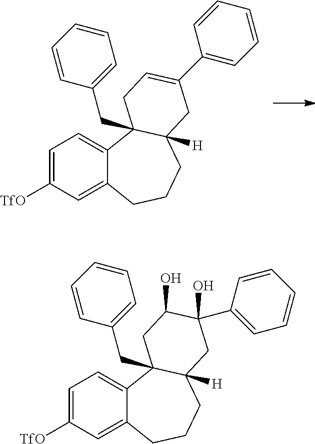

To a flask was added trifluoromethanesulfonic acid (7aS,11aS)-11a-benzyl-9-phenyl-6,7,7a,8,11,11a-hexahydro-5H-dibenzo[a,c]cyclohepten-3-yl ester compound with trifluoromethanesulfonic acid (7aR,11aR)-11a-benzyl-9-phenyl-6,7,7a,8,11,11a-hexahydro-5H-dibenzo[a,c]cyclohepten-3-yl ester (61, R²=Benzyl, R³=Phenyl) (0.500 g, 0.975 mmol), 2.5% osmium tetroxide in t-butanol (0.61 mL, 0.049 mmol) and NMO (0.114 g, 0.975 mmol) followed by the addition of 1,4-dioxane (6 mL) and water (2 mL). The mixture was stirred at rt for about 17 h. The solution was quenched with sodium thiosulfate solution (15 mL) and extracted with DCM (2×15 mL). The organics were dried over MgSO₄, filtered and concentrated under reduced pressure. The residue was purified on silica gel (12 g) eluting with 40% EtOAc in heptane followed by a second purification on silica gel (12 g) eluting with 0 to 20% EtOAc in heptane to provide trifluoromethanesulfonic acid (7aS,9R,10R,11aR)-11a-benzyl-9,10-dihydroxy-9-phenyl-6,7,7a,8,9,10,11,11a-octahydro-5H-dibenzo[a,c]cyclohepten-3-yl ester compound with trifluoro-methanesulfonic acid (7aR,9S,10S,11aS)-11a-benzyl-9,10-dihydroxy-9-phenyl-6,7,7a,8,9,10,11,11a-octahydro-5H-dibenzo[a,c]cyclohepten-3-yl ester (62, R²=Benzyl, R³=Phenyl) (0.293 g, 55%). LC/MS, method 4, Rₜ=2.59 min, MS m/z 605 (M+OAc)⁻. ¹H NMR (400 MHz, DMSO-d₆) δ 7.35 (d, J=3.2 Hz, 1H), 7.01-7.08 (m, 9H), 6.94 (d, J=9.2 Hz, 1H), 6.56-6.51 (m, 2H), 4.55 (s, 1H), 4.38 (d, J=6.0 Hz, 1H), 3.91-3.83 (m, 1H), 3.56 (d, J=13.0 Hz, 1H), 3.29-3.20 (m, 1H), 3.06-2.96 (m, 1H), 2.69-2.54 (m, 2H), 2.08-2.00 (m, 1H), 1.90-1.79 (m, 1H), 1.76-1.83 (m, 2H), 1.56-1.38 (m, 2H), 1.37-1.28 (m, 1H).

### Step 3: (2R,3R,4aS,11bR)-11b-Benzyl-3-phenyl-2,3,4,4a,5,6,7,11b-octahydro-1H-dibenzo[a,c]cycloheptene-2,3,9-triol compound with (2S,3S,4aR,11bS)-11b-benzyl-3-phenyl-2,3,4,4a,5,6,7,11b-octahydro-1H-dibenzo[a,c]cycloheptene-2,3,9-triol (63, R²=Benzyl, R³=Phenyl)

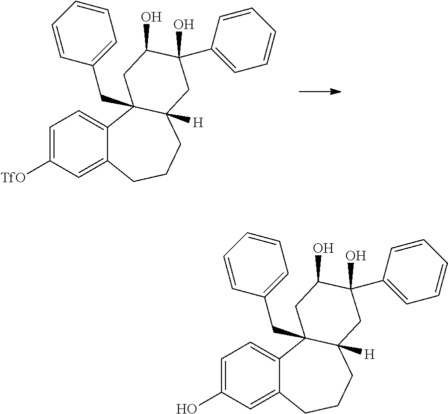

To a flask was added trifluoromethanesulfonic acid (7aS,9R,10R,11aR)-11a-benzyl-9,10-dihydroxy-9-phenyl-6,7,7a,8,9,10,11,11a-octahydro-5H-dibenzo[a,c]cyclohepten-3-yl ester compound with trifluoromethanesulfonic acid (7aR,9S,10S,11aS)-11a-benzyl-9,10-dihydroxy-9-phenyl-6,7,7a,8,9,10,11,11a-octahydro-5H-dibenzo[a,c]cyclohepten-3-yl ester (62, R²=Benzyl, R³=Phenyl) (0.050 g, 0.091 mmol) and tetrabutylammonium hydroxide (0.120 mL, 0.183 mmol) in 1,4-dioxane (3 mL) and the mixture was stirred for about 2 h. The reaction mixture was quenched with a drop of 1 N aqueous HCl and diluted with DCM (5 mL). The organics were separated, dried over MgSO₄, concentrated in vacuo and then purified on silica gel (12 g), eluting with 0-40% EtOAc in heptane. The product containing fractions were partially concentrated until solids precipitated. The solids were collected by filtration and dried under reduced pressure to provide (2R,3R,4aS,11bR)-11b-benzyl-3-phenyl-2,3,4,4a,5,6,7,11b-octahydro-1H-dibenzo[a,c]cycloheptene-2,3,9-triol; compound with (2S,3S,4aR,11bS)-11b-benzyl-3-phenyl-2,3,4,4a,5,6,7,11b-octahydro-1H-dibenzo[a,c]cycloheptene-2,3,9-triol (63, R²=Benzyl, R³=Phenyl) (0.006 g, 16%). LC/MS, method 2, Rₜ=2.52 min, MS m/z 473 (M+OAc)⁻. ¹H NMR (400 MHz, DMSO-d₆) δ 9.10 (s, 1H), 7.20-7.09 (m, 4H), 7.11-7.04 (m, 4H), 6.62-6.54 (m, 4H), 6.43-6.38 (m, 1H), 4.40 (s, 1H), 4.27 (d, J=6.1 Hz, 1H), 4.00-3.91 (m, 1H), 3.49 (d, J=12.8 Hz, 1H), 3.05-2.98 (m, 1H), 2.79-2.69 (m, 1H), 2.53 (d, J=12.8 Hz, 1H), 2.40-2.32 (m, 1H), 1.98-1.90 (m, 1H), 1.51-1.73 (m, 4H), 1.48-1.35 (m, 1H), 1.30-1.21 (m, 2H).

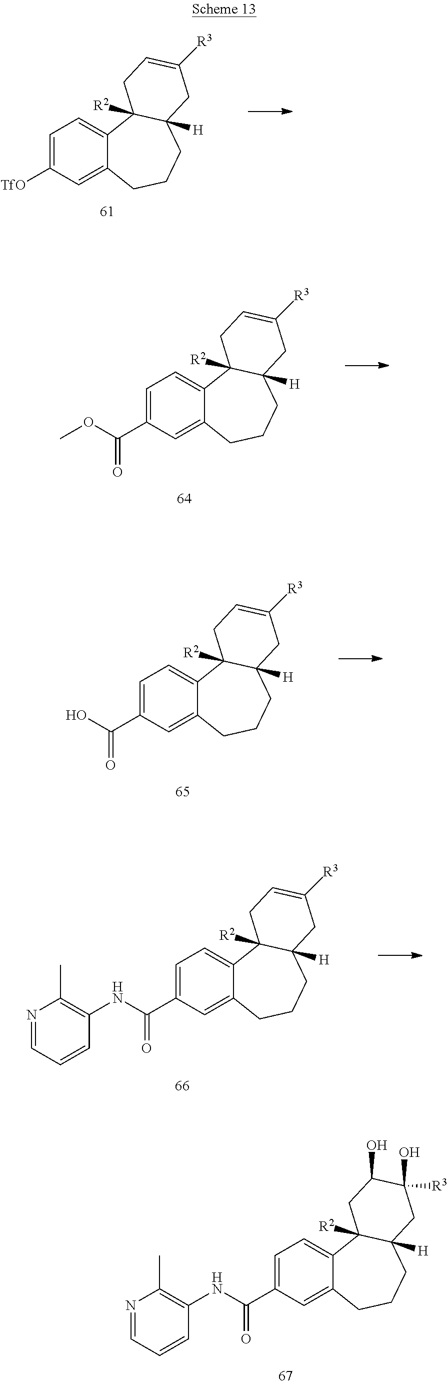

### Example #43

### (7aS,9R,10R,11aR)-11a-Benzyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide; compound with (7aR,9S,10S,11aS)-11a-benzyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide (67, R²=Benzyl, R³=Phenyl)

### Step 1: (7aS,11aS)-11a-Benzyl-9-phenyl-6,7,7a,8,11,11a-hexahydro-5H-dibenzo[a,c]cycloheptene-3-carboxylic acid methyl ester; compound with (7aR,11aR)-11a-benzyl-9-phenyl-6,7,7a,8,11,11a-hexahydro-5H-dibenzo[a,c]cycloheptene-3-carboxylic acid methyl ester (64, R²=Benzyl, R³=Phenyl)

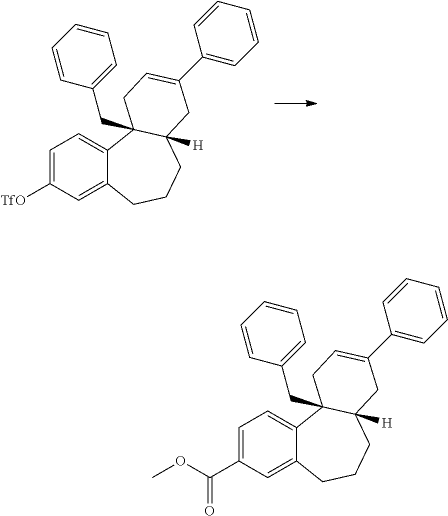

Trifluoro-methanesulfonic acid (7aS,11aS)-11a-benzyl-9-phenyl-6,7,7a,8,11,11a-hexahydro-5H-dibenzo[a,c]cyclohepten-3-yl ester; compound with trifluoro-methanesulfonic acid (7aR,11aR)-11a-benzyl-9-phenyl-6,7,7a,8,11,11a-hexahydro-5H-dibenzo[a,c]cyclohepten-3-yl ester (61, R²=Benzyl, R³=Phenyl) (0.428 g, 0.835 mmol) and PdCl₂(dppf) (0.061 g, 0.083 mmol) were combined under nitrogen, followed by the addition of DMF (5 mL) and the mixture was degassed by bubbling a stream of nitrogen for about 10 min. The reaction mixture was briefly evacuated and an atmosphere of CO was added from a balloon. MeOH (0.34 mL, 8.4 mmol) and TEA (0.23 mL, 1.7 mmol) were added and the reaction was heated at about 90° C. for about 2 h. The reaction was cooled to rt and concentrated in vacuo. The residue was purified on silica gel (25 g), eluting with a gradient of 0-20% EtOAc in heptane to yield (7aS,11aS)-11a-benzyl-9-phenyl-6,7,7a,8,11,11a-hexahydro-5H-dibenzo[a,c]cycloheptene-3-carboxylic acid methyl ester; compound with (7aR,11aR)-11a-benzyl-9-phenyl-6,7,7a,8,11,11a-hexahydro-5H-dibenzo[a,c]cycloheptene-3-carboxylic acid methyl ester (64, R²=Benzyl, R³=Phenyl) as a white solid (0.156 g, 44%). LC/MS, method 4, Rₜ=3.00 min, MS m/z 423 (M+H)⁺.

### Step 2: (7aS,11aS)-11a-Benzyl-9-phenyl-6,7,7a,8,11,11a-hexahydro-5H-dibenzo[a,c]cycloheptene-3-carboxylic acid; compound with (7aR,11aR)-11a-benzyl-9-phenyl-6,7,7a,8,11,11a-hexahydro-5H-dibenzo[a,c]cycloheptene-3-carboxylic acid (65, R²=Benzyl, R³=Phenyl)

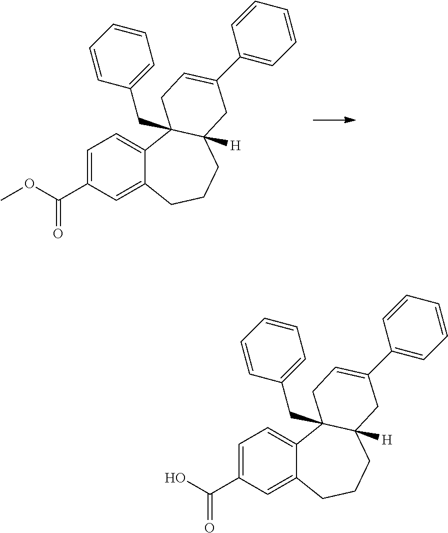

To a round flask was added (7aS,11aS)-11a-benzyl-9-phenyl-6,7,7a,8,11,11a-hexahydro-5H-dibenzo[a,c]cycloheptene-3-carboxylic acid methyl ester; compound with (7aR,11aR)-11a-benzyl-9-phenyl-6,7,7a,8,11,11a-hexahydro-5H-dibenzo[a,c]cycloheptene-3-carboxylic acid methyl ester (64, R²=Benzyl, R³=Phenyl) (0.156 g, 0.37 mmol) and LiOH (0.081 g, 3.3 mmol) [Alfa Aesar] in 1,4-dioxane (2 mL) and water (1 mL) and the suspension was stirred at about 75° C. for about 60 h. The reaction was concentrated under reduced pressure, then acidified with 1N aqueous HCl. Water (5 mL) was added and the resulting suspension was collected by filtration to provide (7aS,11aS)-11a-benzyl-9-phenyl-6,7,7a,8,11,11a-hexahydro-5H-dibenzo[a,c]cycloheptene-3-carboxylic acid; compound with (7aR,11aR)-11a-benzyl-9-phenyl-6,7,7a,8,11,11a-hexahydro-5H-dibenzo[a,c]cycloheptene-3-carboxylic acid (65, R²=Benzyl, R³=Phenyl) (0.151 g, 100%). LC/MS, method 4, Rₜ=2.53 min, MS m/z 407 (M−H)⁻.

### Step 3: (7aS,11aS)-11a-Benzyl-9-phenyl-6,7,7a,8,11,11a-hexahydro-5H-dibenzo[a,c]cycloheptene-3-carboxylic acid (2-methyl-pyridin-3-yl)-amide; compound with (7aR,11aR)-11a-benzyl-9-phenyl-6,7,7a,8,11,11a-hexahydro-5H-dibenzo[a,c]cycloheptene-3-carboxylic acid (2-methyl-pyridin-3-yl)-amide (66, R²=Benzyl, R³=Phenyl)

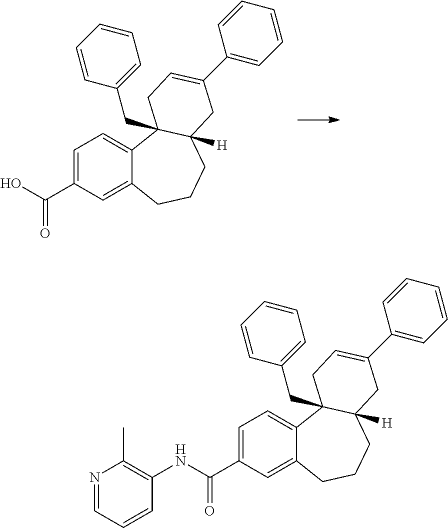

To a round flask was added (7aS,11aS)-11a-benzyl-9-phenyl-6,7,7a,8,11,11a-hexahydro-5H-dibenzo[a,c]cycloheptene-3-carboxylic acid; compound with (7aR,11aR)-11a-benzyl-9-phenyl-6,7,7a,8,11,11a-hexahydro-5H-dibenzo[a,c]cycloheptene-3-carboxylic acid (65, R²=Benzyl, R³=Phenyl) (0.150 g, 0.367 mmol) and DIEA (0.064 mL, 0.37 mmol) in THF (6 mL). TFFH (0.097 g, 0.37 mmol) was added and mixture was stirred at rt for about 10 min. 2-Methylpyridin-3-amine (0.079 g, 0.73 mmol) was then added and the mixture was heated to about 60° C. for about 18 h. Additional 2-methylpyridin-3-amine (0.020 g, 0.18 mmol) was added followed by TFFH (0.015 g, 0.055 mmol). The mixture was stirred at about 60° C. for about 18 h. Solvents were removed under reduced pressure and the residue was purified on a silica gel (12 g), eluting with a gradient of 0-100% EtOAc in heptane to give (7aR,11aR)-11a-benzyl-9-phenyl-6,7,7a,8,11,11a-hexahydro-5H-dibenzo[a,c]cycloheptene-3-carboxylic acid (2-methyl-pyridin-3-yl)-amide; compound with (7aS,11aS)-11a-benzyl-9-phenyl-6,7,7a,8,11,11a-hexahydro-5H-dibenzo[a,c]cycloheptene-3-carboxylic acid (2-methyl-pyridin-3-yl)-amide (66, R²=Benzyl, R³=Phenyl) (0.108 g, 59%). LC/MS, method 2, Rₜ=3.38 min, MS m/z 499 (M+H)⁺.

### Step 4: (7aR,9S,10S,11aS)-11a-Benzyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide; compound with (7aS,9R,10R,11aR)-11a-benzyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide (67, R²=Benzyl, R³=Phenyl)

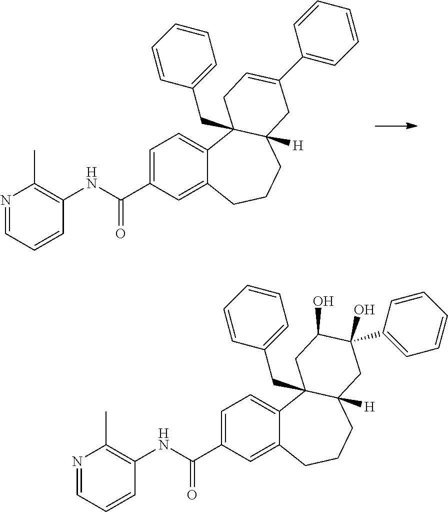

To a round flask was added (7aR,11aR)-11a-benzyl-9-phenyl-6,7,7a,8,11,11a-hexahydro-5H-dibenzo[a,c]cycloheptene-3-carboxylic acid (2-methyl-pyridin-3-yl)-amide; compound with (7aS,11aS)-11a-benzyl-9-phenyl-6,7,7a,8,11,11a-hexahydro-5H-dibenzo[a,c]cycloheptene-3-carboxylic acid (2-methyl-pyridin-3-yl)-amide (66, R²=Benzyl, R³=Phenyl) (0.103 g, 0.207 mmol) and osmium tetroxide (0.13 mL, 0.010 mmol, 2.5% solution in tert-butanol) in 1,4-dioxane (3 mL) and water (1 mL). NMO (0.024 g, 0.21 mmol) was then added and the mixture was stirred at rt for about 18 h. Additional osmium tetroxide (0.13 mL, 0.010 mmol, 2.5% solution in tert-butanol) and NMO (0.024 g, 0.21 mmol) were added and the mixture was stirred for about 18 h. Aqueous sodium thiosulfate solution (5 mL) was added and the mixture was extracted with DCM (10 mL). The organic layer was dried over MgSO₄, filtered and concentrated under reduced pressure. The residue was purified on silica gel (12 g), eluting with a gradient of 50-100% EtOAc in heptane. Product fractions were combined and concentrated under reduced pressure and the residue was triturated with 50% Et₂O in heptane (5 mL). The residue was purified by reverse phase HPLC eluting with 30-100% MeCN in 50 mM NH₄OAc buffer solution to afford (7aS,9R,10R,11aR)-11a-benzyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide; compound with (7aR,9S,10S,11aS)-11a-benzyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide (67, R²=Benzyl, R³=Phenyl) (0.014 g, 13%). LC/MS, method 2, Rₜ=2.27 min, MS m/z 533 (M+H)⁺. ¹H NMR (400 MHz, DMSO-d₆) δ 9.97 (m, 1H), 8.34 (dd, J=4.71, 1.6 Hz, 1H), 7.90-7.85 (m, 1H), 7.79-7.75 (m, 1H), 7.66-7.60 (m, 1H), 7.28 (dd, J=7.9, 4.8 Hz, 1H), 7.23-7.01 (m, 9H), 6.65-6.59 (m, 2H), 4.53 (s, 1H), 4.41 (d, J=5.9 Hz, 1H), 4.01-3.94 (m, 1H), 3.62-3.56 (m, 1H), 3.12-3.00 (m, 1H), 2.73-2.64 (m, 1H), 2.63-2.57 (m, 1H), 2.48-2.45 (m, 4H), 2.16-2.08 (m, 1H), 1.89-1.75 (m, 3H), 1.75-1.66 (m, 1H), 1.57-1.45 (m, 2H), 1.36-1.30 (m, 1H).

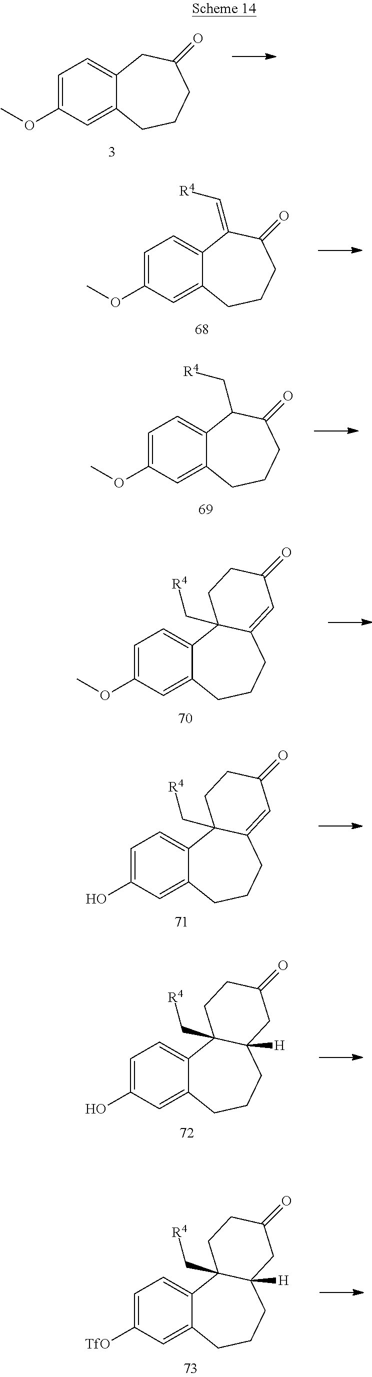

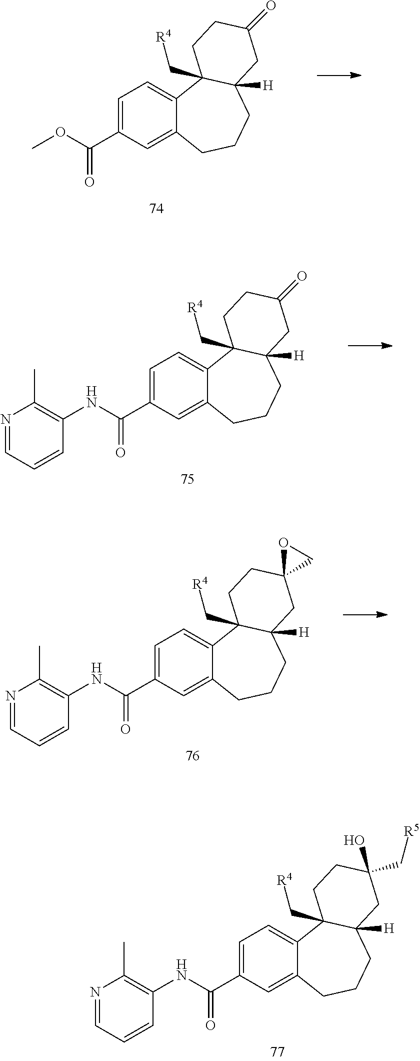

### Example #44 and #45

### (7aS,9R,11aR)-11a-Ethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide (77, R⁴=Methyl, R⁵=Ethyl) and (7aR,9S,11aS)-11a-ethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide (77, R⁴=Methyl, R⁵=Ethyl)

### Step #1: 5-Eth-(E)-ylidene-2-methoxy-5,7,8,9-tetrahydro-benzocyclohepten-6-one (68, R⁴=Methyl)

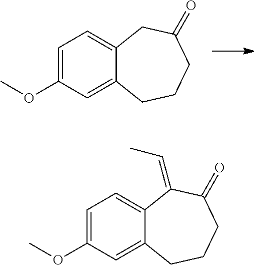

A solution of 2-methoxy-8,9-dihydro-5H-benzo[7]annulen-6(7H)-one (3) (11.3 g, 59.4 mmol) in THF (225 mL) was cooled to about −78° C. under nitrogen. LiHMDS (1 M solution in THF, 59.4 mL, 59.4 mmol) was added dropwise, maintaining reaction temperature below −75° C. When the addition was complete, the reaction was warmed to about 0° C. for about 5 min. The reaction was cooled to about −78° C. and acetaldehyde (4.7 mL, 83 mmol) was added in one portion. The mixture was stirred for about 30 min at about −78° C., then the reaction was allow to warm to rt over about 1 h. The reaction was quenched with saturated aqueous NaCl (500 mL) and extracted with EtOAc (500 mL). The organic layer was washed with saturated aqueous NaCl (500 mL), dried over Na₂SO₄, filtered and concentrated to an oil. The crude oil was purified on silica gel (330 g) using a gradient of 10-30% EtOAc in heptane. Product fractions were combined, concentrated to solids and dried under reduced pressure to yield 5-eth-(E)-ylidene-2-methoxy-5,7,8,9-tetrahydro-benzocyclohepten-6-one (68, R⁴=Methyl) (8.50 g, 66%) as a white solid. LC/MS, method 4, Rₜ=1.88 min, MS m/z 217 (M+H)⁺. ¹H NMR (400 MHz, DMSO-d₆) δ 7.12 (d, J=8.2 Hz, 1H), 7.00-6.93 (m, 1H), 6.90-6.82 (m, 2H), 3.77 (s, 3H), 2.60 (t, J=7.2 Hz, 2H), 2.24 (t, J=7.1 Hz, 2H), 1.93-1.85 (m, 2H), 1.79 (d, J=7.4 Hz, 3H).

### Step #2: 5-Ethyl-2-methoxy-5,7,8,9-tetrahydro-benzocyclohepten-6-one (69, R⁴=Methyl)

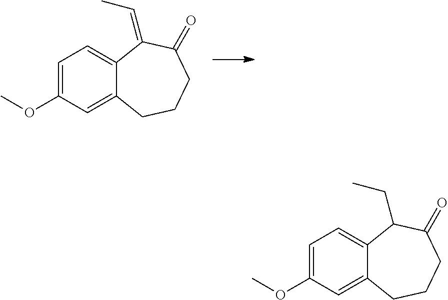

A solution of 5-eth-(E)-ylidene-2-methoxy-5,7,8,9-tetrahydro-benzocyclohepten-6-one (68, R⁴=Methyl) (8.50 g, 39.3 mmol) in toluene (100 mL) containing 20% Pd(OH)₂ on carbon (0.552 g) was evacuated and placed under hydrogen. The reaction was shaken under about 40 psi of hydrogen for about 1 h, then the catalyst was removed by filtration through Celite®, rinsing with toluene (about 20 mL) and the filtrate concentrated under reduced pressure. The residue was further dried under reduced pressure to yield an oil which solidified over time to yield 5-ethyl-2-methoxy-5,7,8,9-tetrahydro-benzocyclohepten-6-one (69, R⁴=Methyl) (8.46 g, 99%) as a white solid. LC/MS, method 4, Rₜ=1.89 min, MS m/z 219 (M+H)⁺. ¹H NMR (400 MHz, DMSO-d₆) δ 7.04-6.99 (m, 1H), 6.77-6.72 (m, 2H), 3.85-3.78 (m, 1H), 3.70 (s, 3H), 3.08-2.97 (m, 1H), 2.81-2.71 (m, 1H), 2.71-2.62 (m, 1H), 2.40-2.32 (m, 1H), 2.12-1.94 (m, 2H), 1.72-1.55 (m, 2H), 0.82 (t, J=7.3 Hz, 3H).

### Step #3: 11b-Ethyl-9-methoxy-1,2,5,6,7,11b-hexahydro-dibenzo[a,c]cyclohepten-3-one (70, R⁴=Methyl)

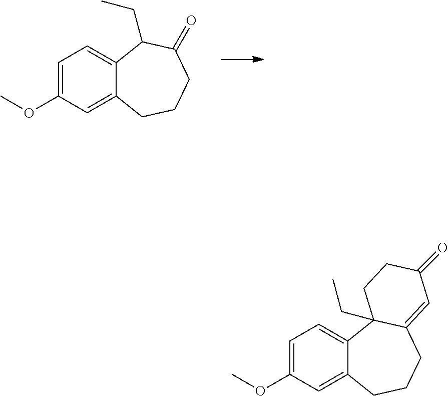

To EtOH (150 mL) under nitrogen was added freshly cut sodium (2.21 g, 96.0 mmol) portionwise and the mixture was stirred until the reaction was complete. A solution of 5-ethyl-2-methoxy-5,7,8,9-tetrahydro-benzocyclohepten-6-one (69, R⁴=Methyl) (14.0 g, 64.1 mmol) in EtOH (150 mL) was added and the mixture was heated to about 60° C. Methyl vinyl ketone (5.82 mL, 70.5 mmol) was added dropwise over about 25 min, and then the reaction was continued for about 2-3 h. The reaction was cooled to rt and concentrated under reduced pressure. The residue was dissolved in EtOAc (200 mL) and washed with saturated aqueous NaCl (2×100 mL), dried over Na₂SO₄, filtered and concentrated under reduced pressure. The crude product was purified on silica gel (330 g) using a gradient of 10-35% EtOAc in heptane. Product fractions were combined and concentrated to a yellow oil which solidified on standing to yield 11b-ethyl-9-methoxy-1,2,5,6,7,11b-hexahydro-dibenzo[a,c]cyclohepten-3-one (70, R⁴=Methyl) (12.2 g, 70%) as a yellow solid. LC/MS, method 4, Rₜ=1.92 min, MS m/z 271 (M+H)⁺. ¹H NMR (400 MHz, DMSO-d₆) δ 7.24 (d, J=8.8 Hz, 1H), 6.80 (dd, J=8.7, 2.9 Hz, 1H), 6.71 (d, J=2.9 Hz, 1H), 5.82 (s, 1H), 3.72 (s, 3H), 2.85-2.75 (m, 1H), 2.67-2.51 (m, 2H), 2.45-2.37 (m, 1H), 2.33-2.19 (m, 3H), 2.14-1.93 (m, 2H), 1.87-1.67 (m, 3H), 0.79 (t, J=7.4 Hz, 3H).

### Alternative step 3: (R)-11b-Ethyl-9-methoxy-1,2,5,6,7,11b-hexahydro-dibenzo[a,c]cyclohepten-3-one; compound with (S)-11b-ethyl-9-methoxy-1,2,5,6,7,11b-hexahydro-dibenzo[a,c]cyclohepten-3-one (71, R⁴=Methyl)

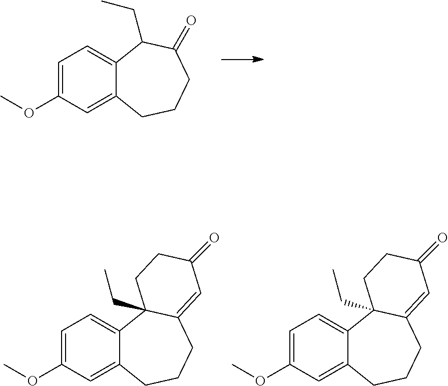

### Step 3a: (1S,4S,8R)-1-(2-Fluoro-4-(trifluoromethyl)benzyl)-2-((S)-hydroxy(quinolin-4-yl)methyl)-8-vinyl-1-azoniabicyclo[2.2.2]octane bromide

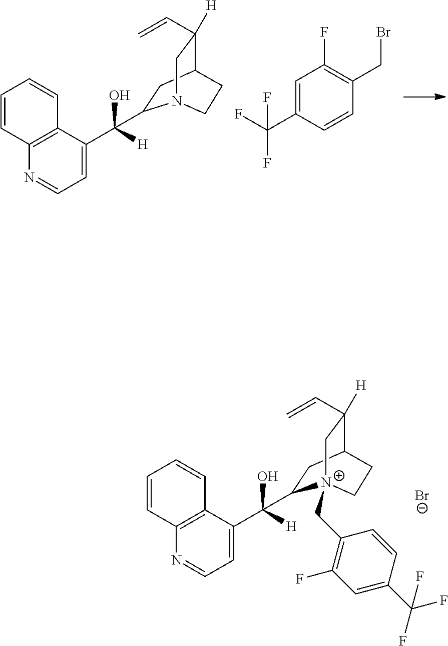

Step 3a was carried out according to the methods described by Wim Nerinckx and Maurits Vandewalle in Tetrahedron: Asymmetry, Vol. 1, No. 4, pp. 265-276, 1990. Thus, Cinchonine (˜85%, rest dihydrocinchonine) (1.0 g, 3.40 mmol) and 1-(bromomethyl)-2-fluoro-4-(trifluoromethyl)benzene (0.960 g, 3.74 mmol) in toluene (20 mL) was heated to about 110° C. for about 3 h. The mixture was allowed to cool to rt. The solids were collected by filtration then washed with toluene (90 mL). The material was dried under vacuum at about 60° C. to give (1S,4S,8R)-1-(2-fluoro-4-(trifluoromethyl)benzyl)-2-((S)-hydroxy(quinolin-4-yl)methyl)-8-vinyl-1-azoniabicyclo[2.2.2]octane bromide (1.75 g, 93%)

### Step 3b: (S)-5-ethyl-2-methoxy-5-(3-oxobutyl)-8,9-dihydro-5H-benzo[7]annulen-6(7H)-one; compound with (S)-5-ethyl-2-methoxy-5-(3-oxobutyl)-8,9-dihydro-5H-benzo[7]annulen-6(7H)-one

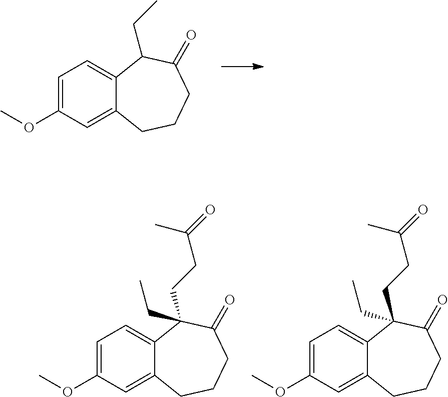

A mixture of toluene (60 mL), KOH (60 wt % in water) (1.713 g, 18.32 mmol) and (1S,4S,8R)-1-(2-fluoro-4-(trifluoromethyl)benzyl)-2-((S)-hydroxy(quinolin-4-yl)methyl)-8-vinyl-1-azoniabicyclo[2.2.2]octane bromide (0.252 g, 0.458 mmol) was stirred at rt for about 16 h. 5-Ethyl-2-methoxy-8,9-dihydro-5H-benzo[7]annulen-6(7H)-one (1 g, 4.58 mmol) was added and stirring continued for about 1 h. The mixture was cooled to about 0° C. then treated with but-3-en-2-one (0.595 g, 8.49 mmol). After about 2 h additional but-3-en-2-one (0.048 g, 0.687 mmol) was added and stirring continued for about 1 h.

The mixture was treated with EtOAc (20 mL) and 6N HCl (10 mL). The layers were separated then the organic layer was washed with saturated aqueous NaCl (15 mL). The organic layer was dried over MgSO₄, filtered and evaporated. The material was purified on silica gel (40 g) using a gradient from 0% to 40% EtOAc in heptane. Pure product fractions were concentrated to yield (S)-5-ethyl-2-methoxy-5-(3-oxobutyl)-8,9-dihydro-5H-benzo[7]annulen-6(7H)-one; compound with (S)-5-ethyl-2-methoxy-5-(3-oxobutyl)-8,9-dihydro-5H-benzo[7]annulen-6(7H)-one (0.770 g, 58.3%). LC/MS, method 3, Rₜ=2.27 min, MS m/z 289 (M+H)⁺

### Step 3c: (R)-11b-Ethyl-9-methoxy-1,2,5,6,7,11b-hexahydro-dibenzo[a,c]cyclohepten-3-one; compound with (S)-11b-ethyl-9-methoxy-1,2,5,6,7,11b-hexahydro-dibenzo[a,c]cyclohepten-3-one (71, R⁴=Methyl)

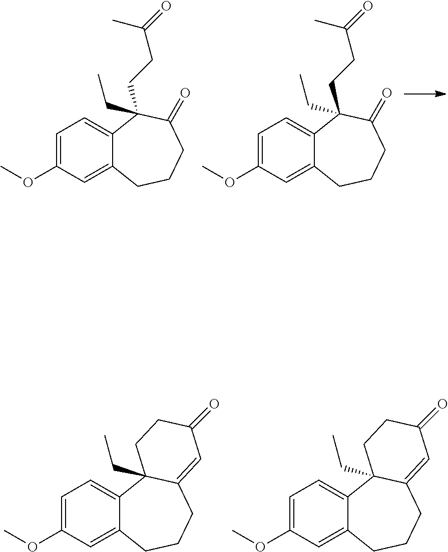

Sodium (0.092 g, 4.00 mmol) was dissolved in EtOH (7 mL) with heating to about 60° C. The solution was added to (S)-5-ethyl-2-methoxy-5-(3-oxobutyl)-8,9-dihydro-5H-benzo[7]annulen-6(7H)-one; compound with (S)-5-ethyl-2-methoxy-5-(3-oxobutyl)-8,9-dihydro-5H-benzo[7]annulen-6(7H)-one (0.770 g, 2.67 mmol) from Step 2 in EtOH (7 mL) then the mixture was warmed to about 60° C. for about 2 h. The mixture was cooled to rt and concentrated under reduced pressure. The material was partitioned between EtOAc (25 mL) and water (25 ml). 6 N HCl was added to make the aqueous layer acidic (˜pH 3) then the layers were separated. The organic layer was dried over MgSO₄, filtered and concentrated under reduced pressure. The residue was purified on silica gel (12 g) using a gradient of 0% to 100% EtOAc in heptane. Fractions containing product were concentrated under reduced pressure to yield (R)-11b-ethyl-9-methoxy-1,2,5,6,7,11b-hexahydro-dibenzo[a,c]cyclohepten-3-one; compound with (S)-11b-ethyl-9-methoxy-1,2,5,6,7,11b-hexahydro-dibenzo[a,c]cyclohepten-3-one (71, R⁴=Methyl) (0.600 g, 83%). The residue was treated with isopropyl acetate (2.4 g) then the mixture was briefly heated in an oil bath at about 90° C. until the material dissolved. The solution was cooled to about 35° C. then seeded with crystals of (R)-11b-ethyl-9-methoxy-1,2,5,6,7,11b-hexahydro-dibenzo[a,c]cyclohepten-3-one. The mixture was stirred at rt overnight then the mixture was cooled to about 0° C. for about 45 min. The solids were collected by filtration and washed with MeOH (˜0.25 mL). The material was dried under vacuum at about 65° C. to yield (R)-11b-ethyl-9-methoxy-1,2,5,6,7,11b-hexahydro-dibenzo[a,c]cyclohepten-3-one (0.300 g, 41.6%). LC/MS, method 3, Rₜ=2.39 min, MS m/z 271 (M+H)⁺. Chiral SFC method D, Rₜ=4.08 min, 100% by ELSD.

### Step #4: 11b-Ethyl-9-hydroxy-1,2,5,6,7,11b-hexahydro-dibenzo[a,c]cyclohepten-3-one (71, R⁴=Methyl)

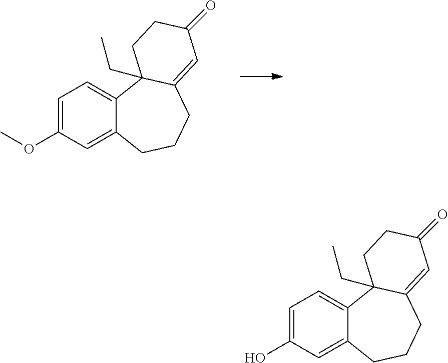

A mixture containing 11b-ethyl-9-methoxy-1,2,5,6,7,11b-hexahydro-dibenzo[a,c]cyclohepten-3-one (70, R⁴=Methyl) (10.2 g, 37.7 mmol) and DL-methionine (18.3 g, 123 mmol) in methanesulfonic acid (100 mL, 1.54 mol) was mechanically stirred under nitrogen at rt over about 3 days. The reaction was diluted with DCM (700 mL) and poured carefully onto ice water (700 mL). The layers were separated and the aqueous layer was extracted with DCM (500 mL). The combined organic layers were washed with water (2×500 mL), dried over Na₂SO₄, filtered and concentrated under reduced pressure. The residue was purified on silica gel (220 g) using a gradient of 0-50% EtOAc in DCM. Product fractions were combined and concentrated under reduced pressure to yield 11b-ethyl-9-hydroxy-1,2,5,6,7,11b-hexahydro-dibenzo[a,c]cyclohepten-3-one (71, R⁴=Methyl) (8.54 g, 88%) as an off-white solid. LC/MS, method 4, Rₜ=1.32 min, MS m/z 257 (M+H)⁺. ¹H NMR (400 MHz, DMSO-d₆) δ 9.19 (s, 1H), 7.11 (d, J=8.6 Hz, 1H), 6.62 (dd, J=8.5, 2.7 Hz, 1H), 6.51 (d, J=2.7 Hz, 1H), 5.81 (s, 1H), 2.79-2.70 (m, 1H), 2.67-2.52 (m, 1H), 2.44-2.35 (m, 2H), 2.33-2.18 (m, 3H), 2.14-2.04 (m, 1H), 2.01-1.90 (m, 1H), 1.86-1.66 (m, 3H), 0.78 (t, J=7.4 Hz, 3H).

### Step #5: (4aS,11bR)-11b-Ethyl-9-hydroxy-1,2,4,4 a,5,6,7,11b-octahydro-dibenzo[a,c]cyclohepten-3-one; compound with (4aR,11bS)-11b-ethyl-9-hydroxy-1,2,4,4a,5,6,7,11b-octahydro-dibenzo[a,c]cyclohepten-3-one (72, R⁴=Methyl)

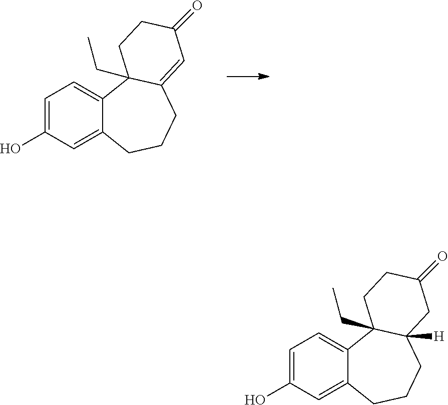

To a suspension of 11b-ethyl-9-hydroxy-1,2,5,6,7,11b-hexahydro-dibenzo[a,c]cyclohepten-3-one (71, R⁴=Methyl) (11.3 g, 43.9 mmol) and 10% Pd on C (1.40 g) in THF (80 mL) was added pyridine (20 mL) and the mixture was hydrogenated at rt under about 40 psi of hydrogen for about 18 h. The catalyst was removed by filtration through Celite®, rinsing with THF (20 mL) and the filtrate was concentrated. The residue was dissolved in DCM (200 mL) and washed with 2 N aqueous HCl (100 mL), dried over Na₂SO₄, filtered and concentrated under reduced pressure. The residue was re-dissolved in EtOAc (100 mL) and DCM (100 mL), filtered through a short pad of silica gel, and concentrated until product began to precipitate. Product was collected by filtration, rinsed with EtOAc (10 mL) and dried under reduced pressure to yield (4aS,11bR)-11b-ethyl-9-hydroxy-1,2,4,4a,5,6,7,11b-octahydro-dibenzo[a,c]cyclohepten-3-one; compound with (4aR,11bS)-11b-ethyl-9-hydroxy-1,2,4,4a,5,6,7,11b-octahydro-dibenzo[a,c]cyclohepten-3-one (72, R⁴=Methyl) (6.45 g, 57%) as a white solid. LC/MS, method 4, Rₜ=1.32 min, MS m/z 257 (M+H)⁺. ¹H NMR (400 MHz, DMSO-d₆) δ 9.14 (s, 1H), 7.11-7.05 (m, 1H), 6.60-6.54 (m, 2H), 2.96-2.86 (m, 1H), 2.65-2.54 (m, 2H), 2.47-2.36 (m, 1H), 2.29-2.20 (m, 1H), 2.20-2.05 (m, 4H), 1.89-1.79 (m, 1H), 1.71-1.51 (m, 3H), 1.49-1.31 (m, 2H), 0.61 (t, J=7.4 Hz, 3H).

### Step #6: Trifluoro-methanesulfonic acid (7aR,11aS)-11a-ethyl-9-oxo-6,7,7a,8,9,10,11,11a-octahydro-5H-dibenzo[a,c]cyclohepten-3-yl ester; compound with trifluoro-methanesulfonic acid (7aS,11aR)-11a-ethyl-9-oxo-6,7,7a,8,9,10,11,11a-octahydro-5H-dibenzo[a,c]cyclohepten-3-yl ester (73, R⁴=Methyl)

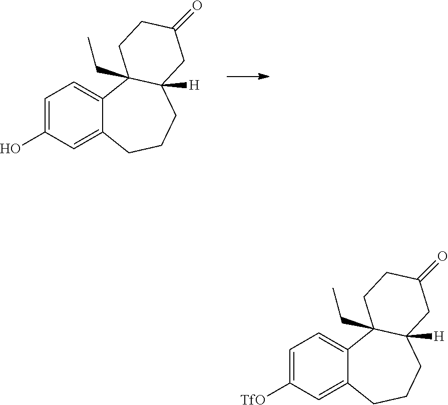

A solution of (4aS,11bR)-11b-ethyl-9-hydroxy-1,2,4,4a,5,6,7,11b-octahydro-dibenzo[a,c]cyclohepten-3-one; compound with (4aR,11bS)-11b-ethyl-9-hydroxy-1,2,4,4a,5,6,7,11b-octahydro-dibenzo[a,c]cyclohepten-3-one (72, R⁴=Methyl) (6.45 g, 25.0 mmol) in DCM (100 mL) was treated with N-phenylbis(trifluoromethanesulfonimide) (8.92 g, 25.0 mmol) and DIEA (8.7 mL, 50 mmol) at rt. The reaction was stirred at rt for about 72 h. Silica gel (30 g) was added and solvents were removed under reduced pressure. The residue was loaded on silica gel (220 g) and purified using a gradient of 10-30% EtOAc in heptane. Product fractions were combined and concentrated to yield trifluoro-methanesulfonic acid (7aR,11aS)-11a-ethyl-9-oxo-6,7,7a,8,9,10,11,11a-octahydro-5H-dibenzo[a,c]cyclohepten-3-yl ester; compound with trifluoro-methanesulfonic acid (7aS,11aR)-11a-ethyl-9-oxo-6,7,7a,8,9,10,11,11a-octahydro-5H-dibenzo[a,c]cyclohepten-3-yl ester (73, R⁴=Methyl) (8.82 g, 90%) as an oil. LC/MS, method 4, Rₜ=2.53 min, MS m/z 449 (M+OAc)⁻. ¹H NMR (400 MHz, DMSO-d₆) δ 7.48 (d, J=8.8 Hz, 1H), 7.29 (d, J=2.9 Hz, 1H), 7.25 (dd, J=8.7, 2.9 Hz, 1H), 3.05-2.95 (m, 1H), 2.91-2.82 (m, 1H), 2.68-2.59 (m, 1H), 2.44-2.24 (m, 2H), 2.24-2.11 (m, 3H), 2.08-1.96 (m, 1H), 1.94-1.86 (m, 1H), 1.78-1.64 (m, 2H), 1.61-1.51 (m, 1H), 1.51-1.37 (m, 2H), 0.59 (t, J=7.4 Hz, 3H).

### Step #7: (7aR,11aS)-11a-Ethyl-9-oxo-6,7,7a,8,9,10,11,11a-octahydro-5H-dibenzo[a,c]cycloheptene-3-carboxylic acid methyl ester; compound with (7aS,11aR)-11a-ethyl-9-oxo-6,7,7a,8,9,10,11,11a-octahydro-5H-dibenzo[a,c]cycloheptene-3-carboxylic acid methyl ester (74, R⁴=Methyl)

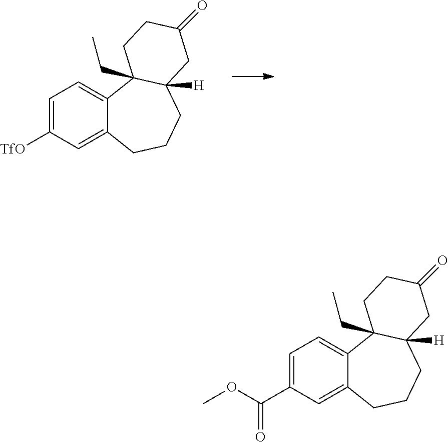

A solution of trifluoro-methanesulfonic acid (7aR,11aS)-11a-ethyl-9-oxo-6,7,7a,8,9,10,11,11a-octahydro-5H-dibenzo[a,c]cyclohepten-3-yl ester; compound with trifluoro-methanesulfonic acid (7aS,11aR)-11a-ethyl-9-oxo-6,7,7a,8,9,10,11,11a-octahydro-5H-dibenzo[a,c]cyclohepten-3-yl ester (73, R⁴=Methyl) (6.12 g, 15.7 mmol) in DMF (65 mL) was treated with Xantphos (0.907 g, 1.57 mmol) and Pd₂(dba)₃ (0.431 g, 0.470 mmol). The mixture was purged with a stream of nitrogen for about 10 min. The reaction was evacuated briefly and then an atmosphere of carbon monoxide was introduced with a balloon. To the mixture was added MeOH (3.8 mL, 94 mmol) and then TEA (4.4 mL, 31 mmol) and the mixture was heated at about 100° C. for about 18 h. The reaction was cooled to rt and concentrated under reduced pressure. The residue was purified on silica gel (220 g) using a gradient of 10 to 40% EtOAc in heptane. Product fractions were combined and concentrated under reduced pressure to yield (7aR,11aS)-11a-ethyl-9-oxo-6,7,7a,8,9,10,11,11a-octahydro-5H-dibenzo[a,c]cycloheptene-3-carboxylic acid methyl ester; compound with (7aS,11aR)-11a-ethyl-9-oxo-6,7,7a,8,9,10,11,11a-octahydro-5H-dibenzo[a,c]cycloheptene-3-carboxylic acid methyl ester (74, R⁴=Methyl) (3.10 g, 66%) as an oil. LC/MS, method 4, Rₜ=2.17 min, no parent mass. ¹H NMR (400 MHz, DMSO-d₆) δ 7.77-7.71 (m, 2H), 7.47 (d, J=8.2 Hz, 1H), 3.81 (s, 3H), 3.09-2.07 (m, 1H), 2.93-2.82 (m, 1H), 2.73-2.63 (m, 1H), 2.46-2.35 (m, 1H), 2.34-2.24 (m, 1H), 2.24-2.11 (m, 3H), 2.07-1.95 (m, 1H), 1.94-1.84 (m, 1H), 1.78-1.62 (m, 2H), 1.63-1.52 (m, 1H), 1.52-1.38 (m, 2H), 0.60 (t, J=7.4 Hz, 3H).

### Step #8: (7aR,11aS)-11a-Ethyl-9-oxo-6,7,7a,8,9,10,11,11a-octahydro-5H-dibenzo[a,c]cycloheptene-3-carboxylic acid (2-methyl-pyridin-3-yl)-amide; compound with (7aS,11aR)-11a-ethyl-9-oxo-6,7,7a,8,9,10,11,11a-octahydro-5H-dibenzo[a,c]cycloheptene-3-carboxylic acid (2-methyl-pyridin-3-yl)-amide (75, R⁴=Methyl)

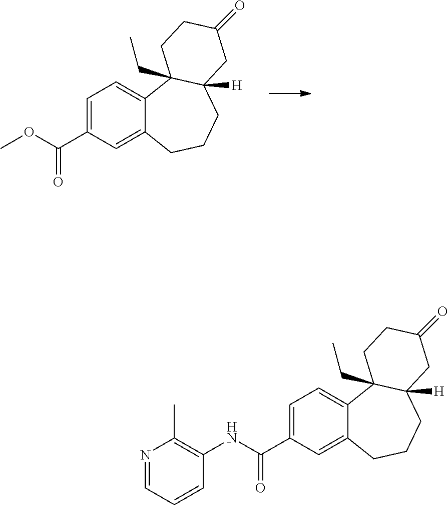

A solution of (7aR,11aS)-11a-ethyl-9-oxo-6,7,7a,8,9,10,11,11a-octahydro-5H-dibenzo[a,c]cycloheptene-3-carboxylic acid methyl ester; compound with (7aS,11aR)-11a-ethyl-9-oxo-6,7,7a,8,9,10,11,11a-octahydro-5H-dibenzo[a,c]cycloheptene-3-carboxylic acid methyl ester (74, R⁴=Methyl) (3.10 g, 10.3 mmol) in 1,4-dioxane (25.0 mL) and was treated with lithium hydroxide monohydrate (1.30 g, 31.0 mmol) and the reaction was stirred at about 70° C. for about 15 min. The reaction was cooled and concentrated. The residue was dissolved in water (50 mL), washed with Et₂O (30 mL), then acidified with 2 N aqueous HCl. The carboxylic acid was extracted with DCM (2×40 mL), dried over Na₂SO₄, filtered and concentrated under reduced pressure. The residue was dissolved in THF (30.0 mL) and treated with DIEA (1.80 mL, 10.3 mmol) and BTFFH (3.26 g, 10.3 mmol). The mixture was stirred for about 5 min, then 2-methylpyridin-3-amine (1.12 g, 10.3 mmol) was added and the mixture was heated at about 60° C. for about 18 h. The mixture was cooled to rt, then additional DIEA and BTFFH were added (about 0.10 equivalents each). The mixture was re-heated to about 60° C. for about 18 h. The reaction was cooled and concentrated under reduced pressure and the residue was dissolved in DCM (50 mL) and washed with saturated aqueous NaHCO₃ (2×50 mL). The organic layer was dried over Na₂SO₄, filtered and concentrated under reduced pressure. The residue was purified on silica gel (120 g) using a gradient of 0-100% EtOAc in DCM. Product fractions were combined and concentrated under reduced pressure. The residue was triturated with EtOAc (20 mL). The product was collected by filteration and dried under reduced pressure to yield (7aR,11aS)-11a-ethyl-9-oxo-6,7,7a,8,9,10,11,11a-octahydro-5H-dibenzo[a,c]cycloheptene-3-carboxylic acid (2-methyl-pyridin-3-yl)-amide; compound with (7aS,11aR)-11a-ethyl-9-oxo-6,7,7a,8,9,10,11,11a-octahydro-5H-dibenzo[a,c]cycloheptene-3-carboxylic acid (2-methyl-pyridin-3-yl)-amide (75, R⁴=Methyl) (2.66 g, 68%) as an off-white solid. LC/MS, method 4, Rₜ=2.17 min, no parent mass. ¹H NMR (400 MHz, DMSO-d₆) δ 9.98 (s, 1H), 8.30 (dd, J=4.7, 1.5 Hz, 1H), 7.82-7.75 (m, 2H), 7.71 (dd, J=8.0, 1.6 Hz, 1H), 7.47 (d, J=8.2 Hz, 1H), 7.25 (dd, J=8.0, 4.8 Hz, 1H), 3.13-3.00 (m, 1H), 2.95-2.84 (m, 1H), 2.77-2.67 (m, 1H), 2.46-2.38 (m, 4H), 2.36-2.27 (m, 1H), 2.27-2.14 (m, 3H), 2.13-2.00 (m, 1H), 1.95-1.87 (m, 1H), 1.81-1.65 (m, 2H), 1.65-1.40 (m, 3H), 0.64 (t, J=7.4 Hz, 3H).

### Step #9: (+/−) Compound 76 (R⁴=Methyl)

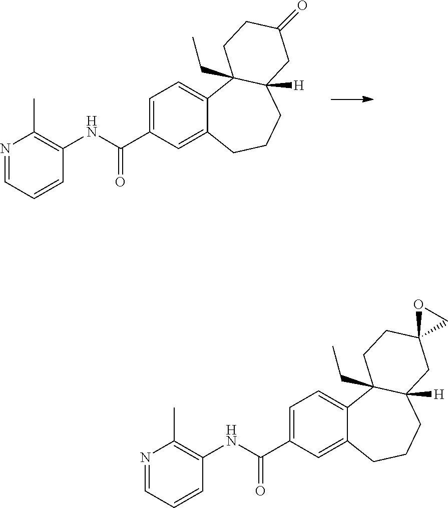

To sodium hydride (60% dispersion in mineral oil, 0.563 g, 14.1 mmol) under nitrogen was added DMSO (32 mL) and the mixture was heated at about 60° C. for about 60 min. The reaction was cooled to about rt, diluted with THF (32 mL) and the mixture was cooled to about 0° C. Trimethylsulfoxonium iodide (3.10 g, 14.1 mmol) was added then the reaction was stirred for about 10 min. A suspension of (7aR,11aS)-11a-ethyl-9-oxo-6,7,7a,8,9,10,11,11a-octahydro-5H-dibenzo[a,c]cycloheptene-3-carboxylic acid (2-methyl-pyridin-3-yl)-amide; compound with (7aS,11aR)-11a-ethyl-9-oxo-6,7,7a,8,9,10,11,11a-octahydro-5H-dibenzo[a,c]cycloheptene-3-carboxylic acid (2-methyl-pyridin-3-yl)-amide (75, R⁴=Methyl) (2.65 g, 7.04 mmol) in THF (32 mL) was added while maintaining the reaction temperature below 4° C., and then the reaction was allowed to warm to rt for about 18 h. Solvents were removed under reduced pressure and the residue was diluted with EtOAc (200 mL) and washed with water (2×200 mL). The organic layer was dried over Na₂SO₄, filtered and concentrated to about 20 mL under reduced pressure. Heptane was added to turbidity (about 10 mL) and the mixture was allowed to stand for about 30 min. The precipitate was collected by filtration, rinsed with 50% EtOAc in heptane (20 mL) and dried under reduced pressure to yield (+/−) Compound 76 (R⁴=Methyl) (2.29 g, 83%) as an off-white solid. LC/MS, method 2, Rₜ=2.31 min, MS m/z 391 (M+H)⁺. ¹H NMR (400 MHz, DMSO-d₆) δ 9.94 (s, 1H), 8.32 (dd, J=4.7, 1.5 Hz, 1H), 7.82-7.65 (m, 3H), 7.41 (d, J=8.3 Hz, 1H), 7.25 (dd, J=7.9, 4.7 Hz, 1H), 3.08-2.96 (m, 1H), 2.94-2.83 (m, 1H), 2.58-2.49 (m, 3H), 2.42 (s, 3H), 2.30-2.05 (m, 4H), 1.76-1.38 (m, 6H), 1.22-1.12 (m, 1H), 0.83-0.73 (m, 1H), 0.64 (t, J=7.4 Hz, 3H).

### Step #10: (7aR,9R,11aS)-11a-Ethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide; compound with (7aS,9R,11aR)-11a-ethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide (77, R⁴=Methyl, R⁵=Ethyl)

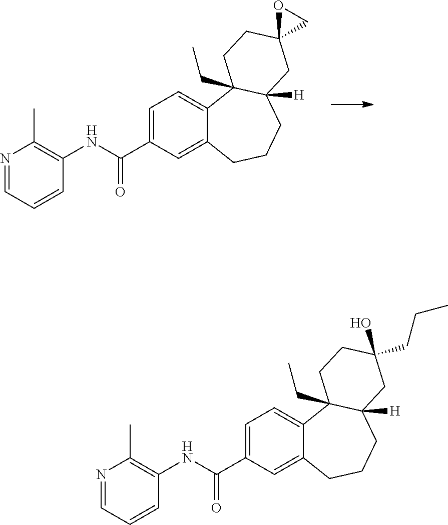

A round bottom flask with stirring bar, septum, nitrogen line and thermometer was charged with (+/−) Compound 76 (R⁴=Methyl) (200 mg, 0.512 mmol), THF (6.4 mL) and copper (I) iodide (9.8 mg, 0.051 mmol). The mixture was cooled to an internal temperature of about 0° C. then ethylmagnesium bromide (3M solution in Et₂O, 1.0 mL, 3.0 mmol) was added dropwise maintaining reaction temperature between 0° C. and 5° C. The mixture was stirred for about 1 h at about 0° C., and then the reaction was quenched by addition of saturated aqueous NH₄Cl (20 mL) and EtOAc (30 mL). The mixture was stirred at rt for about 1 h, then the organic layer was removed and stirred again with saturated aqueous NH₄Cl (20 mL) for about 15 min. The layers were separated and the organic layer was dried over Na₂SO₄, filtered and concentrated under reduced pressure. The residue was purified on silica gel (12 g) using a gradient of 70-100% EtOAc in heptane. Product fractions were combined and concentrated under reduced pressure to yield (7aR,9R,11aS)-11a-ethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide; compound with (7aS,9R,11aR)-11a-ethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide (77, R⁴=Methyl, R⁵=Ethyl) as a solid (185 mg, 86%). LC/MS, method 2, Rₜ=2.34 min, MS m/z 421 (M+H)⁺. ¹H NMR (400 MHz, DMSO-d₆) δ 9.92 (s, 1H), 8.31 (dd, J=4.7, 1.6 Hz, 1H), 7.75-7.67 (m, 3H), 7.35 (d, J=8.4 Hz, 1H), 7.25 (dd, J=7.9, 4.8 Hz, 1H), 3.89 (s, 1H), 3.02-2.93 (m, 1H), 2.90-2.80 (m, 1H), 2.42 (s, 3H), 2.26-2.15 (m, 3H), 2.10-1.99 (m, 1H), 1.73-1.60 m, 2H), 1.54-1.37 (m, 5H), 1.26-1.15 (m, 2H), 1.15-1.04 (m, 4H), 0.75 (t, J=7.1 Hz, 3H), 0.60 (t, J=7.4 Hz, 3H).

Chiral separation of (77, R⁴=Methyl, R⁵=Ethyl)

Purification Method: (SFC) Isocratic, 27% co-solvent B (80 mL/min, 100 bar system pressure, 25° C.). Co-solvent B was 1:1 HPLC grade MeOH:isopropanol. Solvent A was SFC grade CO₂. The column used for the chromatography was a 30×250 mm RegisPack from Regis Technologies (5 μm particles). The first peak eluted was (7aR,9S,11aS)-11a-ethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide (Example 44) and the second was (7aS,9R,11aR)-11a-ethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide (Example 45). NMR and LCMS data for single isomers was essentially identical to the racemic mixture.

Additional examples, prepared in a manner similar to the preparation of Examples #44 and #45, are listed in Table 1.

TABLE 1

| | | | | | | Chiral |
|---|---|---|---|---|---|---|
| | | | | | | method/ |
| | | Grignard | | LC/MS | LC/MS R_{T}/ | Order of |
| Ex.# | Epoxide | Rgt. | Product structure | method | MH⁺ | elution |
| 46 | Compound | Methylmagnesium- | Compound 77 | 2 | 2.18 min | 2/First |
| | 76 (R⁴ = | bromide | (7aR,9S,11aS) (R⁴ = | | 407 | |
| | Methyl) | | Methyl) (R⁵ = | | | |
| | | | Methyl) | | | |
| 47 | Compound | Methylmagnesium- | Compound 77 | 2 | 2.18 min | 2/ |
| | 76 (R⁴ = | bromide | (7aS,9R,11aR) (R⁴ = | | 407 | Second |
| | Methyl) | | Methyl) (R⁵ = | | | |
| | | | Methyl) | | | |
| 48 | Compound | Isopropylmagnesium- | Compound 77 | 2 | 2.51 min | 3/First |
| | 76 (R⁴ = | bromide | (7aR,9R,11aS) | | 435 | |
| | Methyl) | | (R⁴ = Methyl) (R⁵ = | | | |
| | | | Isopropyl) | | | |
| 49 | Compound | Isopropylmagnesium- | Compound 77 | 2 | 2.51 min | 3/ |
| | 76 (R⁴ = | bromide | (7aS,9S,11aR) (R⁴ = | | 435 | Second |
| | Methyl) | | Methyl) (R⁵ = | | | |
| | | | Isopropyl) | | | |
| 50 | Compound | Cyclopropyl | Compound 77 | 2 | 2.36 min | 4/First |
| | 76 (R⁴ = | magnesium- | (7aR,9R,11aS) | | 433 | |
| | Methyl) | bromide | (R⁴ = Methyl) (R⁵ = | | | |
| | | | Cyclopropyl) | | | |
| 51 | Compound | Cyclopropyl | Compound 77 | 2 | 2.36 min | 4/ |
| | 76 (R⁴ = | magnesium- | (7aS,9S,11aR) (R⁴ = | | 433 | Second |
| | Methyl) | bromide | Methyl) (R⁵ = | | | |
| | | | Cyclopropyl) | | | |
| 52 | Compound | Ethylmagnesium | Compound 77 | 2 | 2.24 min | NA |
| | 76 (R⁴ = | bromide | (7aR,9S,11aS): | | 475 | |
| | Trifluoromethyl) | | compound with | | | |
| | | | (7aS,9R,11aR) | | | |
| | | | (R⁴ = | | | |
| | | | Trifluoromethyl) | | | |
| | | | (R⁵ = ethyl) | | | |
| 53 | Compound | Isopropylmagnesium- | Compound 152 | 2 | 2.26 min. | NA |
| | 151 (R⁶ = 2- | bromide | (7aS,9S,11aR); | | 437 | |
| | Methylpyridin- | | compound with | | | |
| | 3-yl, R⁸ = | | (7aR,9R,11aS) (R⁵ = | | | |
| | H, R⁹ = H) | | Isopropyl, R⁶ = | | | |
| | | | 2-Methylpyridin- | | | |
| | | | 3-yl, R⁸ = H, R⁹ = | | | |
| | | | H) | | | |
| 54 | Compound | Cyclopropyl | Compound 152 | 2 | 2.12 min. | NA |
| | 151 (R⁶ = 2- | magnesium- | (7aS,9S,11aR); | | 435 | |
| | Methylpyridin- | bromide | compound with | | | |
| | 3-yl, R⁸ = | | (7aR,9R,11aS) (R⁵ = | | | |
| | H, R⁹ = H) | | Cyclopropyl, R⁶ = | | | |
| | | | 2- | | | |
| | | | Methylpyridin-3- | | | |
| | | | yl, R⁸ = H, R⁹ = | | | |
| | | | H) | | | |
| 55 | Compound | Isopropylmagnesium- | Compound 152 | 2 | 2.23 min. | 9/ |
| | 151 (R⁶ = 2- | bromide | (7aS,9S,11aR) (R⁵ = | | 437 | Second |
| | Methylpyridin- | | Isopropyl, R⁶ = | | | |
| | 3-yl, R⁸ = | | 2-Methylpyridin- | | | |
| | H, R⁹ = H) | | 3-yl, R⁸ = H, R⁹ = | | | |
| | | | H) | | | |
| 56 | Compound | Isopropylmagnesium- | Compound 152 | 2 | 2.23 min. | 9/First |
| | 151 (R⁶ = 2- | bromide | (7aR,9R,11aS) (R⁵ = | | 437 | |
| | Methylpyridin- | | Isopropyl, R⁶ = | | | |
| | 3-yl, R⁸ = | | 2-Methylpyridin- | | | |
| | H, R⁹ = H) | | 3-yl, R⁸ = H, R⁹ = | | | |
| | | | H) | | | |
| 57 | Compound | Cyclopropyl | Compound 152 | 2 | 2.11 min. | 11/ |
| | 151 (R⁶ = 2- | magnesium- | (7aS,9S,11aR) (R⁵ = | | 434 | Second |
| | Methylpyridin- | bromide | Cyclopropyl, R⁶ = | | | |
| | 3-yl, R⁸ = | | 2- | | | |
| | H, R⁹ = H) | | Methylpyridin-3- | | | |
| | | | yl, R⁸ = H, R⁹ = | | | |
| | | | H) | | | |
| 58 | Compound | Cyclopropyl | Compound 152 | 2 | 2.11 min. | 11/First |
| | 151 (R⁶ = 2- | magnesium- | (7aR,9R,11aS) (R⁵ = | | 434 | |
| | Methylpyridin- | bromide | Cyclopropyl, R⁶ = | | | |
| | 3-yl, R⁸ = | | 2- | | | |
| | H, R⁹ = H) | | Methylpyridin-3- | | | |
| | | | yl, R⁸ = H, R⁹ = | | | |
| | | | H) | | | |
| 58A | Compound | Ethylmagnesium- | Compound 77 | 2 | 2.24 min | 14/First |
| | 76 (R⁴ = | bromide | (7aR,9S,11aS): | | 475 | |
| | Trifluoromethyl) | | (R⁴ = | | | |
| | | | Trifluoromethyl) | | | |
| | | | (R⁵ = ethyl) | | | |
| 58B | Compound | Ethylmagnesium- | Compound 77 | 2 | 2.24 min | 14/ |
| | 76 (R⁴ = | bromide | (7aS,9R,11aR) | | 475 | Second |
| | Trifluoromethyl) | | (R⁴ = | | | |
| | | | Trifluoromethyl) | | | |
| | | | (R⁵ = ethyl) | | | |

### Example #59 and #60

### (7aR,9S,11aS)-11a-Ethyl-9-hydroxy-9-methyl-6,7,7a,8,9,10,11,11a-octahydro-5H-dibenzo[a,c]cycloheptene-3-carboxylic acid (2-methyl-pyridin-3-yl)-amide (77, R⁴=Methyl, R⁵=H) and (7aS,9R,11aR)-11a-ethyl-9-hydroxy-9-methyl-6,7,7a,8,9,10,11,11a-octahydro-5H-dibenzo[a,c]cycloheptene-3-carboxylic acid (2-methyl-pyridin-3-yl)-amide (77, R⁴=Methyl, R⁵=H)

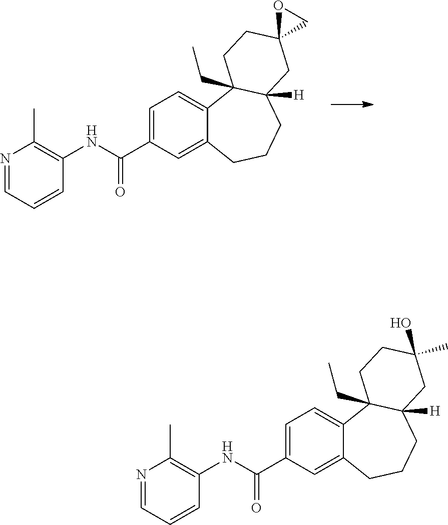

A solution of (+/−) Compound 76 (R⁴=Methyl) (150 mg, 0.384 mmol) in EtOH (3 mL) was treated with sodium borohydride (35 mg, 0.92 mmol) and the reaction was stirred at rt for about 18 h. The reaction was quenched with acetic acid (0.50 mL) and concentrated under reduced pressure. The residue was distributed between EtOAc (15 mL) and saturated aqueous NaHCO₃ (10 mL). The organic layer was dried over Na₂SO₄, filtered and concentrated under reduced pressure. The residue was purified on silica gel (4 g) using EtOAc as eluant. Product fractions were combined and concentrated under reduced pressure to yield (7aR,9S,11aS)-11a-ethyl-9-hydroxy-9-methyl-6,7,7a,8,9,10,11,11a-octahydro-5H-dibenzo[a,c]cycloheptene-3-carboxylic acid (2-methyl-pyridin-3-yl)-amide; compound with (7aS,9R,11aR)-11a-ethyl-9-hydroxy-9-methyl-6,7,7a,8,9,10,11,11a-octahydro-5H-dibenzo[a,c]cycloheptene-3-carboxylic acid (2-methyl-pyridin-3-yl)-amide (77, R⁴=Methyl, R⁵=H) (145 mg, 96%) as a solid. LC/MS, method 2, Rₜ=2.06 min, MS m/z 393 (M+H)⁺. ¹H NMR (400 MHz, DMSO-d₆) δ 9.92 (s, 1H), 8.31 (dd, J=4.8, 1.6 Hz, 1H), 7.75-7.66 (m, 3H), 7.35 (d, J=8.4 Hz, 1H), 7.25 (dd, J=7.9, 4.8 Hz, 1H), 4.06 (s, 1H), 3.03-2.92 (m, 1H), 2.92-2.81 (m, 1H), 2.42 (s, 3H), 2.34-2.15 (m, 3H), 2.10-1.99 (m, 1H), 1.75-1.60 (m, 2H), 1.56-1.37 (m, 5H), 1.15-1.06 (m, 2H), 0.94 (s, 3H), 0.60 (t, J=7.4 Hz, 3H).

### Chiral Separation of (77, R⁴=Methyl, R⁵=H)

Chiral purification Method 2 was used to separate enantiomers. The first peak eluted was (7aR,9S,11aS)-11a-ethyl-9-hydroxy-9-methyl-6,7,7a,8,9,10,11,11a-octahydro-5H-dibenzo[a,c]cycloheptene-3-carboxylic acid (2-methyl-pyridin-3-yl)-amide (Example 59); and the second was (7aS,9R,11aR)-11a-ethyl-9-hydroxy-9-methyl-6,7,7a,8,9,10,11,11a-octahydro-5H-dibenzo[a,c]cycloheptene-3-carboxylic acid (2-methyl-pyridin-3-yl)-amide (Example 60). NMR and LC/MS data for single isomers was essentially identical to the racemic mixture.

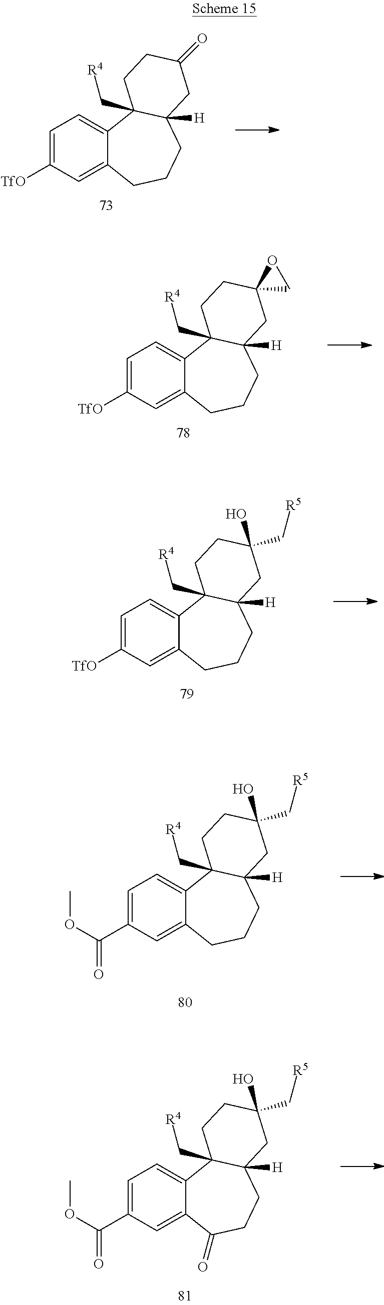

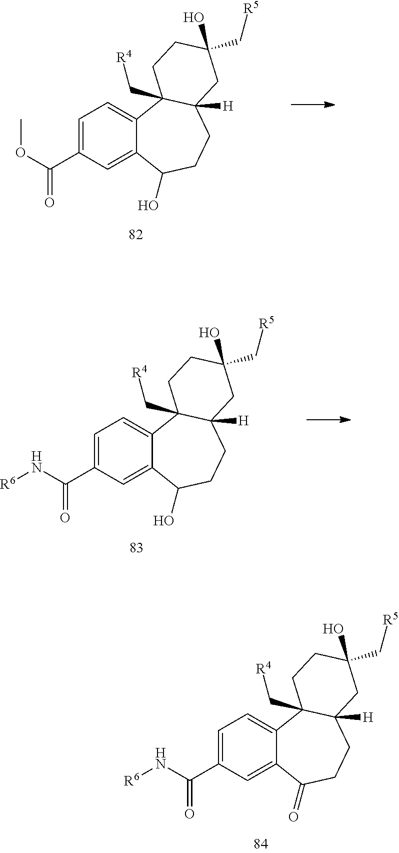

### Example #61 and #62

### (7aR,9R,11aS)-11a-Ethyl-9-hydroxy-5-oxo-9-propyl-6,7,7a,8,9,10,11,11a-octahydro-5H-dibenzo[a,c]cycloheptene-3-carboxylic acid (2-methyl-pyridin-3-yl)-amide (84, R⁴=Methyl, R⁵=Ethyl, R⁶=2-Methyl-3-pyridyl) and (7aS,9R,11aR)-11a-ethyl-9-hydroxy-5-oxo-9-propyl-6,7,7a,8,9,10,11,11a-octahydro-5H-dibenzo[a,c]cycloheptene-3-carboxylic acid (2-methyl-pyridin-3-yl)-amide (84, R⁴=Methyl, R⁵=Ethyl, R⁶=2-Methyl-3-pyridyl)

### Step #1: (+/−) Compound 78 (R⁴=Methyl)

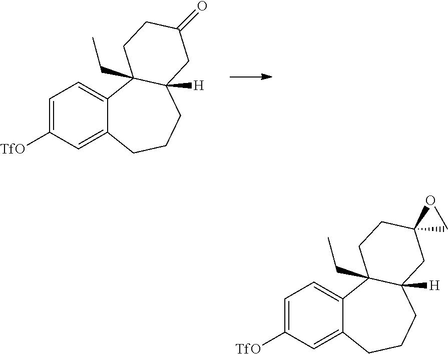

To sodium hydride (60% dispersion in mineral oil, 0.50 g, 12.6 mmol) under nitrogen was added DMSO (39 mL) and the mixture was heated at about 60° C. for about 60 min. The reaction was cooled to rt, diluted with THF (39 mL) and the mixture was cooled to about 0° C. Trimethylsulfoxonium iodide (2.78 g, 12.6 mmol) was added then the reaction was stirred for about 10 min. A solution of trifluoro-methanesulfonic acid (7aR,11aS)-11a-ethyl-9-oxo-6,7,7a,8,9,10,11,11a-octahydro-5H-dibenzo[a,c]cyclohepten-3-yl ester; compound with trifluoro-methanesulfonic acid (7aS,11aR)-11a-ethyl-9-oxo-6,7,7a,8,9,10,11,11a-octahydro-5H-dibenzo[a,c]cyclohepten-3-yl ester (73, R⁴=Methyl) (3.29 g, 8.43 mmol) in THF (39 mL) was added maintaining reaction temperature below 4° C. and then the reaction was allowed to warm to rt. Stirring was continued for about 3 h at rt and then the reaction was quenched by addition of saturated aqueous NH₄Cl (100 mL). The product was extracted with EtOAc (100 mL) and the organic layer was washed with saturated aqueous NaCl (100 mL), dried over Na₂SO₄, filtered and concentrated under reduced pressure. The crude product was purified on silica gel (80 g) using a gradient of 10-30% EtOAc in heptane. Product fractions were combined and concentrated under reduced pressure to yield (+/−) Compound 78 (R⁴=Methyl) (1.65 g, 48%) as a white solid. LC/MS, method 4, Rₜ=2.09 min, MS m/z 403 (M−H)⁻. ¹H NMR (400 MHz, DMSO-d₆) δ 7.44-7.38 (m, 1H), 7.27-7.21 (m, 2H), 3.00-2.90 (m, 1H), 2.89-2.81 (m, 1H), 2.55-2.49 (m, 2H), 2.47-2.41 (m, 1H), 2.27-2.00 (m, 4H), 1.74-1.31 (m, 6H), 1.15 (d, J=14.2 Hz, 1H), 0.77 (d, J=13.1 Hz, 1H), 0.59 (t, J=7.4 Hz, 3H).

### Step #2: Trifluoro-methanesulfonic acid (7aR,9S,11aS)-11a-ethyl-9-hydroxy-9-propyl-6,7,7a,8,9,10,11,11a-octahydro-5H-dibenzo[a,c]cyclohepten-3-yl ester; compound with trifluoro-methanesulfonic acid (7aS,9R,11aR)-11a-ethyl-9-hydroxy-9-propyl-6,7,7a,8,9,10,11,11a-octahydro-5H-dibenzo[a,c]cyclohepten-3-yl ester (79, R⁴=Methyl, R⁵=Ethyl)

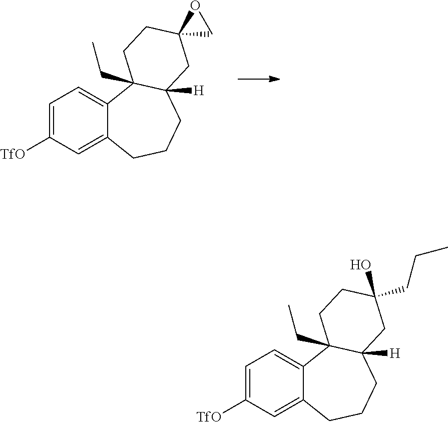

A round bottom flask with stir bar, septum, nitrogen line and thermometer was charged with (+/−) Compound 78 (R⁴=Methyl) (970 mg, 2.40 mmol), THF (50 mL) and copper(I)iodide (45.7 mg, 0.240 mmol). The mixture was cooled to an internal temperature of about 0° C. then ethylmagnesium bromide (3.0 M solution in Et₂O, 1.20 mL, 3.60 mmol) was added dropwise maintaining reaction temperature between 0° C. and 5° C. The reaction was stirred for about 30 min, then quenched by addition of saturated aqueous NH₄Cl (20 mL). The volatiles were substantially removed under reduced pressure. EtOAc (30 mL) was added and the mixture was stirred at rt for about 30 min. The layers were separated and the aqueous layer was extracted again with EtOAc (30 mL). The combined organic layers were washed with saturated aqueous NH₄Cl (20 mL), dried over Na₂SO₄, filtered and concentrated under reduced pressure. The residue was purified on silica gel (25 g) using a gradient of 10 to 30% EtOAc in heptane. Product fractions were combined and concentrated under reduced pressure. The residue was further dried under vacuum to yield trifluoro-methanesulfonic acid (7aR,9S,11aS)-11a-ethyl-9-hydroxy-9-propyl-6,7,7a,8,9,10,11,11a-octahydro-5H-dibenzo[a,c]cyclohepten-3-yl ester; compound with trifluoro-methanesulfonic acid (7aS,9R,11aR)-11a-ethyl-9-hydroxy-9-propyl-6,7,7a,8,9,10,11,11a-octahydro-5H-dibenzo[a,c]cyclohepten-3-yl ester (79, R⁴=Methyl, R⁵=Ethyl) (768 mg, 74%) as an oil. LC/MS, method 4, Rₜ=2.87 min, MS m/z 493 (M+OAc)⁻. ¹H NMR (400 MHz, DMSO-d₆) δ 7.37-7.32 (m, 1H), 7.22-7.17 (m, 2H), 3.90 (s, 1H), 2.96-2.86 (m, 1H), 2.86-2.76 (m, 1H), 2.32-2.11 (m, 3H), 2.05-1.94 (m, 1H), 1.71-1.59 (m, 2H), 1.53-1.31 (m, 5H), 1.26-0.96 (m, 6H), 0.75 (t, J=7.1 Hz, 3H), 0.56 (t, J=7.4 Hz, 3H).

### Step #3: (7aR,9S,11aS)-11a-Ethyl-9-hydroxy-9-propyl-6,7,7a,8,9,10,11,11a-octahydro-5H-dibenzo[a,c]cycloheptene-3-carboxylic acid methyl ester; compound with (7aS,9R,11aR)-11a-ethyl-9-hydroxy-9-propyl-6,7,7a,8,9,10,11,11a-octahydro-5H-dibenzo[a,c]cycloheptene-3-carboxylic acid methyl ester (80, R⁴=Methyl, R⁵=Ethyl)

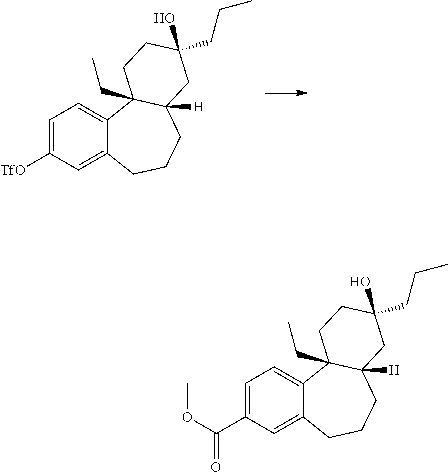

A solution of trifluoro-methanesulfonic acid (7aR,9S,11aS)-11a-ethyl-9-hydroxy-9-propyl-6,7,7a,8,9,10,11,11a-octahydro-5H-dibenzo[a,c]cyclohepten-3-yl ester; compound with trifluoro-methanesulfonic acid (7aS,9R,11aR)-11a-ethyl-9-hydroxy-9-propyl-6,7,7a,8,9,10,11,11a-octahydro-5H-dibenzo[a,c]cyclohepten-3-yl ester (79, R⁴=Methyl, R⁵=Ethyl) (3.00 g, 6.90 mmol) in DMF (28 mL) was treated with Xantphos (0.399 g, 0.690 mmol) and Pd₂(dba)₃ (0.190 g, 0.207 mmol) and the mixture was purged with a stream of nitrogen for about 30 min. The reaction was evacuated briefly and then an atmosphere of carbon monoxide was introduced with a balloon. To the mixture was added MeOH (1.7 mL, 41 mmol) and then TEA (1.9 mL, 14 mmol) and the mixture was heated at about 100° C. for about 18 h. The reaction was cooled to rt and concentrated under reduced pressure. The residue was purified on silica gel (80 g) using a gradient of 10 to 30% EtOAc in heptane. Product fractions were combined and concentrated under reduced pressure to yield (7aR,9S,11aS)-11a-ethyl-9-hydroxy-9-propyl-6,7,7a,8,9,10,11,11a-octahydro-5H-dibenzo[a,c]cycloheptene-3-carboxylic acid methyl ester; compound with (7aS,9R,11aR)-11a-ethyl-9-hydroxy-9-propyl-6,7,7a,8,9,10,11,11a-octahydro-5H-dibenzo[a,c]cycloheptene-3-carboxylic acid methyl ester (80, R⁴=Methyl, R⁵=Ethyl) (1.26 g, 53%) as a colorless glass. LC/MS, method 4, Rₜ=2.87 min, MS m/z 345 (M+H)⁺. ¹H NMR (400 MHz, DMSO-d₆) δ 7.71-7.64 (m, 2H), 7.34 (d, J=8.4 Hz, 1H), 3.88 (s, 1H), 3.80 (s, 3H), 2.98-2.88 (m, 1H), 2.87-2.78 (m, 1H), 2.33-2.13 (m, 3H), 2.03-1.98 (m, 1H), 1.70-1.59 (m, 2H), 1.53-1.34 (m, 5H), 1.20-0.94 (m, 6H), 0.74 (t, J=7.1 Hz, 3H), 0.57 (t, J=7.4 Hz, 3H).

### Step #4: (7aR,9S,11aS)-11a-Ethyl-9-hydroxy-5-oxo-9-propyl-6,7,7a,8,9,10,11,11a-octahydro-5H-dibenzo[a,c]cycloheptene-3-carboxylic acid methyl ester; compound with (7aS,9R,11aR)-11a-ethyl-9-hydroxy-5-oxo-9-propyl-6,7,7a,8,9,10,11,11a-octahydro-5H-dibenzo[a,c]cycloheptene-3-carboxylic acid methyl ester (81, R⁴=Methyl, R⁵=Ethyl)

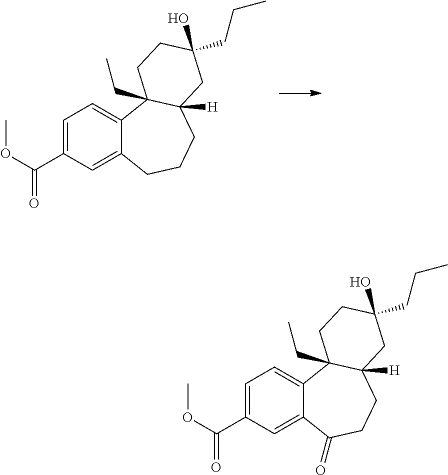

A solution of (7aR,9S,11aS)-11a-ethyl-9-hydroxy-9-propyl-6,7,7a,8,9,10,11,11a-octahydro-5H-dibenzo[a,c]cycloheptene-3-carboxylic acid methyl ester; compound with (7aS,9R,11aR)-11a-ethyl-9-hydroxy-9-propyl-6,7,7a,8,9,10,11,11a-octahydro-5H-dibenzo[a,c]cycloheptene-3-carboxylic acid methyl ester (80, R⁴=Methyl, R⁵=Ethyl) (790 mg, 2.29 mmol) and potassium iodide (0.024 g, 0.46 mmol) in MeCN (30 mL) was heated at about 50° C. and 2-methyl-prop-2-yl-hydroperoxide (5.0 M solution in nonane, 1.7 mL, 8.7 mmol) was added dropwise over a period of about 6 min. The mixture was stirred for about 18 h at about 50° C. The reaction was cooled to rt, diluted EtOAc (30 mL) then washed with 5% aqueous sodium bisulfite solution (30 mL) and with water (30 mL). The organic layer was dried over Na₂SO₄, filtered and concentrated under reduced pressure. The residue was purified on silica gel (40 g) using a gradient of 10-50% EtOAc in heptane. Product fractions were combined and concentrated under reduced pressure to yield (7aS,9R,11aR)-11a-ethyl-9-hydroxy-5-oxo-9-propyl-6,7,7a,8,9,10,11,11a-octahydro-5H-dibenzo[a,c]cycloheptene-3-carboxylic acid methyl ester; compound with (7aR,9S,11aS)-11a-ethyl-9-hydroxy-5-oxo-9-propyl-6,7,7a,8,9,10,11,11a-octahydro-5H-dibenzo[a,c]cycloheptene-3-carboxylic acid methyl ester (81, R⁴=Methyl, R⁵=Ethyl), (134 mg, 16%) as a colorless oil. LC/MS, method 4, Rₜ=2.87 min, MS m/z 345 (M+H)⁺. The crude product was taken to the next step without further purification.

### Step #5: (7aS,9R,11aR)-11a-Ethyl-5,9-dihydroxy-9-propyl-6,7,7a,8,9,10,11,11a-octahydro-5H-dibenzo[a,c]cycloheptene-3-carboxylic acid methyl ester; compound with (7aR,9S,11aS)-11a-ethyl-5,9-dihydroxy-9-propyl-6,7,7a,8,9,10,11,11a-octahydro-5H-dibenzo[a,c]cycloheptene-3-carboxylic acid methyl ester (82, R⁴=Methyl, R⁵=Ethyl)

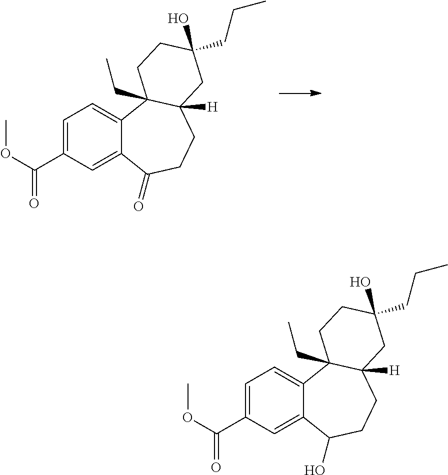

A solution of (7aS,9R,11aR)-11a-ethyl-9-hydroxy-5-oxo-9-propyl-6,7,7a,8,9,10,11,11a-octahydro-5H-dibenzo[a,c]cycloheptene-3-carboxylic acid methyl ester; compound with (7aR,9S,11aS)-11a-ethyl-9-hydroxy-5-oxo-9-propyl-6,7,7a,8,9,10,11,11a-octahydro-5H-dibenzo[a,c]cycloheptene-3-carboxylic acid methyl ester (81, R⁴=Methyl, R⁵=Ethyl) (132 mg, 0.368 mmol) in EtOH (4.0 mL) was stirred at rt and sodium borohydride (28 mg, 0.74 mmol) was added. The reaction was stirred for about 2 h then quenched by careful addition of saturated aqueous NH₄Cl (10 mL) and extracted with EtOAc (2×20 mL). The combined organic extracts were washed with saturated aqueous NaHCO₃ (10 mL), dried over Na₂SO₄, filtered and concentrated under reduced pressure. The residue was purified on silica gel (4 g) using a gradient of 10 to 50% EtOAc in heptane to yield (7aS,9R,11aR)-11a-ethyl-5,9-dihydroxy-9-propyl-6,7,7a,8,9,10,11,11a-octahydro-5H-dibenzo[a,c]cycloheptene-3-carboxylic acid methyl ester; compound with (7aR,9S,11aS)-11a-ethyl-5,9-dihydroxy-9-propyl-6,7,7a,8,9,10,11,11a-octahydro-5H-dibenzo[a,c]cycloheptene-3-carboxylic acid methyl ester (82, R⁴=Methyl, R⁵=Ethyl) as a major isomer (104 mg, 78%) and a minor isomer (16 mg, 12%). Major isomer: LC/MS, method 4, Rₜ=1.50 min, MS m/z 325 (M−H₂O—OH)⁺. ¹H NMR (400 MHz, DMSO-d₆) δ 7.77 (d, J=2.0 Hz, 1H), 7.74 (dd, J=8.2, 2.0 Hz, 1H), 7.37 (d, J=8.4 Hz, 1H), 5.16 (d, J=3.3 Hz, 1H), 4.78-4.73 (m, 1H), 3.86 (s, 1H), 3.81 (s, 3H), 2.66-2.55 (m, 1H), 2.46-2.35 (m, 1H), 2.28-2.16 (m, 2H), 1.84-1.48 (m, 4H), 1.47-1.37 (m, 1H), 1.37-0.93 (m, 8H), 0.72 (t, J=7.1 Hz, 3H), 0.56 (t, J=7.4 Hz, 3H). Minor isomer: LC/MS, method 4, Rₜ=1.45 min, MS m/z 325 (M−H₂O—OH)⁺.

### Step 6: (7aS,9R,11aR)-11a-Ethyl-5,9-dihydroxy-9-propyl-6,7,7a,8,9,10,11,11a-octahydro-5H-dibenzo[a,c]cycloheptene-3-carboxylic acid (2-methyl-pyridin-3-yl)-amide; compound with (7aR,9S,11aS)-11a-ethyl-5,9-dihydroxy-9-propyl-6,7,7a,8,9,10,11,11a-octahydro-5H-dibenzo[a,c]cycloheptene-3-carboxylic acid (2-methyl-pyridin-3-yl)-amide (83, R⁴=Methyl, R⁵=Ethyl, R⁶=2-Methyl-3-pyridyl)

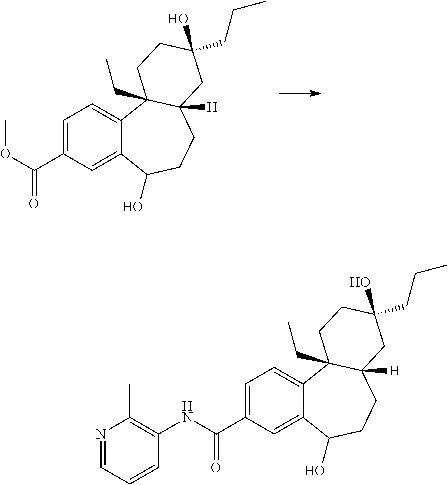

A solution of (7aS,9R,11aR)-11a-ethyl-5,9-dihydroxy-9-propyl-6,7,7a,8,9,10,11,11a-octahydro-5H-dibenzo[a,c]cycloheptene-3-carboxylic acid methyl ester; compound with (7aR,9S,11aS)-11a-ethyl-5,9-dihydroxy-9-propyl-6,7,7a,8,9,10,11,11a-octahydro-5H-dibenzo[a,c]cycloheptene-3-carboxylic acid methyl ester (82, R⁴=Methyl, R⁵=Ethyl) (116 mg, 0.322 mmol) was dissolved in THF (3 mL), 2-methylpyridin-3-amine (38.3 mg, 0.354 mmol) was added and the mixture was cooled to about 0° C. with stirring. LiHMDS (1 M solution in THF, 1.3 mL, 1.3 mmol) was added dropwise and the reaction was stirred for about 30 min. Saturated aqueous NH₄Cl (10 mL) was added and the volatiles were removed under reduced pressure. The mixture was extracted with EtOAc (2×10 mL). The combined organics were washed with saturated aqueous NaHCO₃ (10 mL), dried over Na₂SO₄, filtered and concentrated under reduced pressure. The residue was purified on silica gel (4 g) using EtOAc as eluant. Product fractions were combined and concentrated to yield (7aS,9R,11aR)-11a-ethyl-5,9-dihydroxy-9-propyl-6,7,7a,8,9,10,11,11a-octahydro-5H-dibenzo[a,c]cycloheptene-3-carboxylic acid (2-methyl-pyridin-3-yl)-amide; compound with (7aR,9S,11aS)-11a-ethyl-5,9-dihydroxy-9-propyl-6,7,7a,8,9,10,11,11a-octahydro-5H-dibenzo[a,c]cycloheptene-3-carboxylic acid (2-methyl-pyridin-3-yl)-amide (83, R⁴=Methyl, R⁵=Ethyl, R⁶=2-Methyl-3-pyridyl) (72 mg, 51%) as a glass. LC/MS, method 4, Rₜ=1.09 min, MS m/z 437 (M+H)⁺. The mixture of isomers was taken to the next step without further purification.

### Step 7: (7aS,9R,11aR)-11a-Ethyl-9-hydroxy-5-oxo-9-propyl-6,7,7a,8,9,10,11,11a-octahydro-5H-dibenzo[a,c]cycloheptene-3-carboxylic acid (2-methyl-pyridin-3-yl)-amide; compound with (7aR,9S,11aS)-11a-ethyl-9-hydroxy-5-oxo-9-propyl-6,7,7a,8,9,10,11,11a-octahydro-5H-dibenzo[a,c]cycloheptene-3-carboxylic acid (2-methyl-pyridin-3-yl)-amide (84, R⁴=Methyl, R⁵=Ethyl, R⁶=2-Methyl-3-pyridyl)

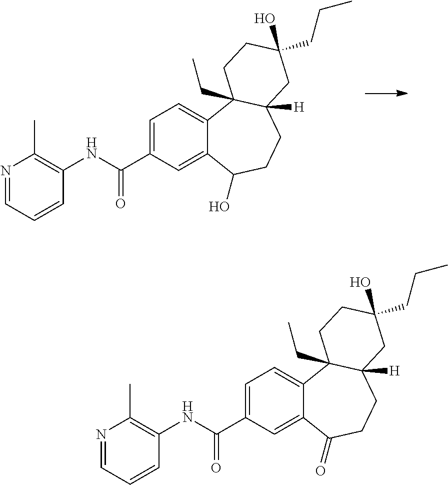

A solution of (7aS,9R,11aR)-11a-ethyl-5,9-dihydroxy-9-propyl-6,7,7a,8,9,10,11,11a-octahydro-5H-dibenzo[a,c]cycloheptene-3-carboxylic acid (2-methyl-pyridin-3-yl)-amide; compound with (7aR,9S,11aS)-11a-ethyl-5,9-dihydroxy-9-propyl-6,7,7a,8,9,10,11,11a-octahydro-5H-dibenzo[a,c]cycloheptene-3-carboxylic acid (2-methyl-pyridin-3-yl)-amide (83, R⁴=Methyl, R⁵=Ethyl, R⁶=2-Methyl-3-pyridyl) (70 mg, 0.16 mmol) in DCM (6 mL) was treated with Dess-Martin periodinane (70 mg, 0.16 mmol) at rt for about 1 h. The reaction was washed with saturated aqueous NaHCO₃ (2×10 mL), dried over Na₂SO₄, filtered and concentrated under reduced pressure. The residue was purified on silica gel (4 g) using a gradient of 90 to 100% EtOAc in heptane. The product fraction was concentrated under reduced pressure then precipitated from Et₂O to yield (7aS,9R,11aR)-11a-ethyl-9-hydroxy-5-oxo-9-propyl-6,7,7a,8,9,10,11,11a-octahydro-5H-dibenzo[a,c]cycloheptene-3-carboxylic acid (2-methyl-pyridin-3-yl)-amide; compound with (7aR,9S,11aS)-11a-ethyl-9-hydroxy-5-oxo-9-propyl-6,7,7a,8,9,10,11,11a-octahydro-5H-dibenzo[a,c]cycloheptene-3-carboxylic acid (2-methyl-pyridin-3-yl)-amide (84, R⁴=Methyl, R⁵=Ethyl, R⁶=2-Methyl-3-pyridyl) (54 mg, 78%) as an off-white solid. LC/MS, method 4, Rₜ=1.19 min, MS m/z 435 (M+H)⁺. ¹H NMR (400 MHz, DMSO-d₆) δ 10.12 (s, 1H), 8.32 (dd, J=4.7, 1.6 Hz, 1H), 8.00 (dd, J=8.3, 2.2 Hz, 1H), 7.86 (d, J=2.2 Hz, 1H), 7.70 (dd, J=8.0, 1.5 Hz, 1H), 7.54 (d, J=8.5 Hz, 1H), 7.26 (dd, J=8.0, 4.7 Hz, 1H), 4.04 (s, 1H), 2.90-2.79 (m, 1H), 2.59-2.51 (m, 1H), 2.46-2.39 (m, 4H), 2.32-2.17 (m, 2H), 1.77-1.67 (m, 1H), 1.63-1.39 (m, 5H), 1.34-1.13 (m, 6H), 0.78 (t, J=6.9 Hz, 3H), 0.55 (t, J=7.4 Hz, 3H).

Chiral Separation of (84, R⁴=Methyl, R⁵=Ethyl, R⁶=2-Methyl-3-pyridyl)

Purification Method: (SFC) Isocratic, 27% co-solvent B (80 mL/min, 100 bar system pressure, 25° C.). Co-solvent B was 1:1 HPLC grade MeOH:isopropanol. Solvent A was SFC grade CO₂. The column used for the chromatography was a 30×250 mm RegisPack from Regis Technologies (5 μm particles). The first peak eluted was (7aS,9R,11aR)-11a-ethyl-9-hydroxy-5-oxo-9-propyl-6,7,7a,8,9,10,11,11a-octahydro-5H-dibenzo[a,c]cycloheptene-3-carboxylic acid (2-methyl-pyridin-3-yl)-amide (84, R⁴=Methyl, R⁵=Ethyl, R⁶=2-Methyl-3-pyridyl) (Example 61) and the second was (7aR,9S,11aS)-11a-ethyl-9-hydroxy-5-oxo-9-propyl-6,7,7a,8,9,10,11,11a-octahydro-5H-dibenzo[a,c]cycloheptene-3-carboxylic acid (2-methyl-pyridin-3-yl)-amide (84, R⁴=Methyl, R⁵=Ethyl, R⁶=2-Methyl-3-pyridyl) (Example 62) NMR and LC/MS data for single isomers was essentially identical to the racemic mixture.

### Example #63

### (7aS,9S,11aS)-9-Hydroxy-9-isobutyl-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-9-hydroxy-9-isobutyl-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide (77, R⁴=Trifluoromethyl, R⁵=isopropyl)

### Step #1: 2-Methoxy-5-(2,2,2-trifluoroethylidene)-8,9-dihydro-5H-benzo[7]annulen-6(7H)-one (68, R⁴=Trifluoromethyl)

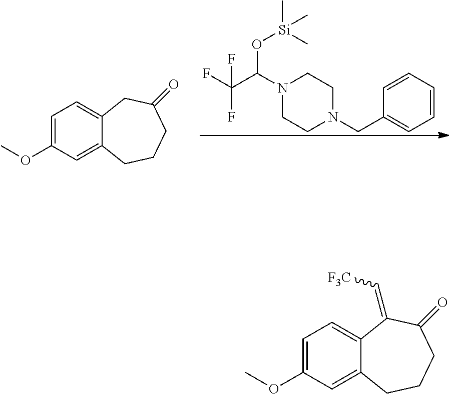

To a solution of 2-methoxy-8,9-dihydro-5H-benzo[7]annulen-6(7H)-one (3) (10.4 g, 54.7 mmol) and 1-benzyl-4-(2,2,2-trifluoro-1-(trimethylsilyloxy)ethyl)piperazine (prepared as described by T. Billaed, B. R. Langlois, and G. Blond, Tetrhedron Letters, 41, (2000) pp. 8777-8780) (19.4 g, 55.8 mmol) in DCE (100 mL) was added boron trifluoride diethyl etherate (9.0 mL, 71 mmol) and the mixture was heated to about 50° C. for about 5 h. The mixture was then cooled to about 0° C. Tfa (33.1 mL, 430 mmol) was added and the mixture was heated to about 60° C. for about 3 h. The mixture was cooled to rt and stirred for about 18 h. The reaction mixture was concentrated under reduced pressure and the residue was purified on silica gel (120 g) eluting with a gradient of 0-50% EtOAc in heptane. Fractions containing product were combined and concentrated under reduced pressure to yield 2-methoxy-5-(2,2,2-trifluoroethylidene)-8,9-dihydro-5H-benzo[7]annulen-6(7H)-one (68, R⁴=Trifluoromethyl) (5.91 g, 41%). LC/MS, method 3, Rₜ=2.73 min, no parent ion. Major isomer: ¹H NMR (600 MHz, DMSO-d₆) δ 7.23-7.18 (m, 1H), 6.95-6.83 (m, 2H), 6.73-6.67 (m, 1H), 3.81 (s, 3H), 2.74-2.69 (m, 2H), 2.44-2.39 (m, 2H), 1.99-1.90 (m, 2H). Minor isomer: ¹H NMR (600 MHz, DMSO-d₆) δ 7.32-7.28 (m, 1H), 6.95-6.83 (m, 2H), 6.17-6.10 (m, 1H), 3.77 (s, 3H), 2.93-2.89 (m, 2H), 2.76-2.71 (m, 2H), 2.02-1.97 (m, 2H).

### Step #2: 2-Methoxy-5-(2,2,2-trifluoroethyl)-8,9-dihydro-5H-benzo[7]annulen-6(7H)-one (69, R⁴=Trifluoromethyl)

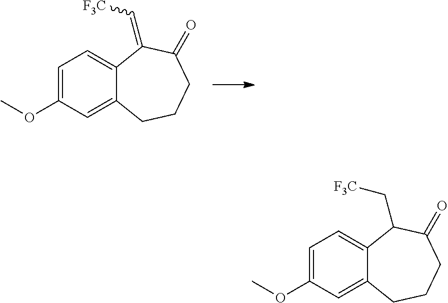

A flask containing 2-methoxy-5-(2,2,2-trifluoroethylidene)-8,9-dihydro-5H-benzo[7]annulen-6(7H)-one (68, R⁴=Trifluoromethyl) (3.34 g, 12.34 mmol) in toluene (25 mL) was evacuated and flushed with N₂. 20% Pd(OH)₂ on carbon (0.607 g) was added. The mixture was evacuated, purged with H₂ and stirred at rt for about 24 h under an atmosphere of H₂. The mixture was flushed with N₂ and the catalyst was removed by filtration through Celite®, rinsing with EtOAc. The filtrate was concentrated under reduced pressure and the residue was purified on silica gel (80 g), eluting with a gradient of 0-60% EtOAc in heptane to yield 2-methoxy-5-(2,2,2-trifluoroethyl)-8,9-dihydro-5H-benzo[7]annulen-6(7H)-one (69, R⁴=Trifluoromethyl) (3.00 g, 89%) as pale yellow crystals. LC/MS, Method 3, Rₜ=2.56 min, MS m/z 271 (M+H)⁺. ¹H NMR (400 MHz, DMSO-d₆) δ 7.06 (d, J=8.4 Hz, 1H), 6.83-6.74 (m, 2H), 4.37 (dd, J=8.9, 4.2 Hz, 1H), 3.73 (s, 3H), 3.28-3.12 (m, 2H), 2.83-2.65 (m, 3H), 2.46-2.39 (m, 1H), 2.17-2.06 (m, 1H), 1.68-1.61 (m, 1H).

### Step #3: 9-Methoxy-11b-trifluoromethyl-1,2,5,6,7,11b-hexahydro-dibenzo[a,c]cyclohepten-3-one (70, R⁴=Trifluoromethyl)

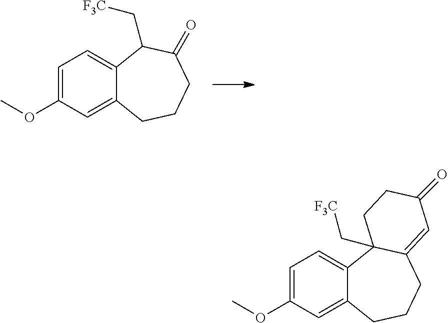

To EtOH (20 mL) was added sodium metal (0.379 g, 16.5 mmol) and the mixture was stirred for about 20 min. A solution of 2-methoxy-5-(2,2,2-trifluoroethyl)-8,9-dihydro-5H-benzo[7]annulen-6(7H)-one (69, R⁴=Trifluoromethyl) (2.99 g, 11.0 mmol) in EtOH (20 mL) was then added and the mixture was heated to about 60° C. Methyl vinyl ketone (1.0 mL, 12 mmol) was then added dropwise, and mixture was heated at about 60° C. for about 2 h, and then stirred at rt for about 18 h. The resulting solids were collected by filtration (crop 1). The filtrate was concentrated under reduced pressure and purified on silica gel (25 g), eluting with a gradient of 5-50% EtOAc in heptane to provide additional product (crop 2). Crops 1 and 2 were combined to yield 9-methoxy-11b-trifluoromethyl-1,2,5,6,7,11b-hexahydro-dibenzo[a,c]cyclohepten-3-one (70, R⁴=Trifluoromethyl) (1.95 g, 55%). LC/MS, method 2, Rₜ=2.50 min, MS m/z 325 (M+H)⁺. ¹H NMR (400 MHz, DMSO-d₆) δ 7.46 (d, J=8.8 Hz, 1H), 6.80 (dd, J=8.7, 2.9 Hz, 1H), 6.72 (d, J=2.9 Hz, 1H), 5.92 (s, 1H), 3.74 (s, 3H), 3.57-3.37 (m, 1H), 3.11-2.96 (m, 1H), 2.89-2.77 (m, 1H), 2.78-2.64 (m, 1H), 2.58-2.42 (m, 2H), 2.39-2.18 (m, 3H), 1.94-1.75 (m, 3H).

### Step #4: 9-Hydroxy-11b-(2,2,2-trifluoro-ethyl)-1,2,5,6,7,11b-hexahydro-dibenzo[a,c]cyclohepten-3-one (71, R⁴=Trifluoromethyl)

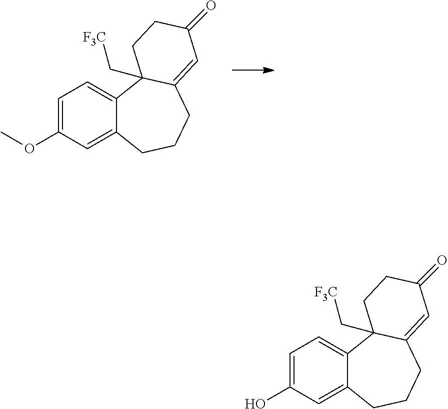

To 9-methoxy-11b-trifluoromethyl-1,2,5,6,7,11b-hexahydro-dibenzo[a,c]cyclohepten-3-one (70, R⁴=Trifluoromethyl) (2.91 g, 8.98 mmol) and DL-methionine (4.35 g, 29.2 mmol) [Alfa Aesar] was added methanesulfonic acid (17.7 mL, 273 mmol) and the mixture was stirred at rt for about 18 h. The mixture was poured slowly into ice water (200 mL) and then DCM (20 mL) was added. The resulting solids were collected by filtration and dried under vacuum (crop 1). The remaining material was extracted with DCM (100 mL), dried over MgSO₄ and concentrated under reduced pressure. The residue was taken into DCM (20 mL) and the solids that formed were collected by filtration and dried under reduced pressure (crop 2). Crops 1 and 2 were combined to yield 9-hydroxy-11b-(2,2,2-trifluoro-ethyl)-1,2,5,6,7,11b-hexahydro-dibenzo[a,c]cyclohepten-3-one (71, R⁴=Trifluoromethyl) (1.78 g, 64%) as an off-white solid. LC/MS, method 3, Rₜ=2.07 min, MS m/z 311 (M+H)⁺

### Step #5: (4aS,11bS)-9-Hydroxy-11b-(2,2,2-trifluoro-ethyl)-1,2,4,4 a,5,6,7,11b-octahydro-dibenzo[a,c]cyclohepten-3-one; compound with (4aR,11bR)-9-hydroxy-11b-(2,2,2-trifluoro-ethyl)-1,2,4,4a,5,6,7,11b-octahydro-dibenzo[a,c]cyclohepten-3-one (72, R⁴=Trifluoromethyl)

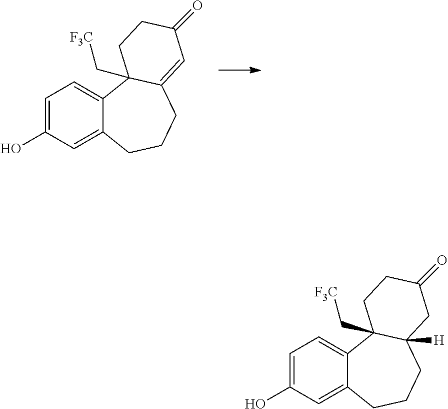

To 9-hydroxy-11b-(2,2,2-trifluoro-ethyl)-1,2,5,6,7,11b-hexahydro-dibenzo[a,c]cyclohepten-3-one (71, R⁴=Trifluoromethyl) (1.14 g, 3.66 mmol) was added pyridine (10 mL) and the mixture was degassed. 10% Pd(OH)₂ on carbon (0.257 g) was added, the mixture was evacuated and hydrogen was introduced via balloon. The mixture was stirred under an atmosphere of H₂ for about 18 h. The reaction was flushed with N₂, then filtered through a Celite® plug (2.0 g), rinsing with EtOAc (20 mL). The filtrate was concentrated under reduced pressure. The residue was purified on silica gel (40 g) eluting with a gradient of 10-60% EtOAc in heptane to provide (4aS,11bS)-9-hydroxy-11b-(2,2,2-trifluoro-ethyl)-1,2,4,4a,5,6,7,11b-octahydro-dibenzo[a,c]cyclohepten-3-one; compound with (4aR,11bR)-9-hydroxy-11b-(2,2,2-trifluoro-ethyl)-1,2,4,4a,5,6,7,11b-octahydro-dibenzo[a,c]cyclohepten-3-one (72, R⁴=Trifluoromethyl) (0.766 g, 67%). LC/MS, method 2, Rₜ=2.18 min, MS m/z 311 (M−H)⁻. ¹H NMR (400 MHz, DMSO-d₆) δ 9.24 (s, 1H), 7.20-7.13 (d, J=9.10 Hz, 1H), 6.61-6.53 (m, 2H), 3.30-3.19 (m, 1H), 2.99-2.88 (m, 1H), 2.86-2.75 (m, 1H), 2.69-2.57 (m, 1H), 2.46-2.34 (m, 2H), 2.35-2.25 (m, 2H), 2.24-2.17 (m, 1H), 2.02-2.13 (m, 1H), 1.96-1.83 (m, 2H), 1.72-1.62 (m, 1H), 1.62-1.52 (m, 1H), 1.49-1.35 (m, 1H).

### Step #6: Trifluoro-methanesulfonic acid (7aS,11aS)-9-oxo-11a-(2,2,2-trifluoro-ethyl)-6,7,7a,8,9,10,11,11a-octahydro-5H-dibenzo[a,c]cyclohepten-3-yl ester; compound with trifluoro-methanesulfonic acid (7aR,11aR)-9-oxo-11a-(2,2,2-trifluoro-ethyl)-6,7,7a,8,9,10,11,11a-octahydro-5H-dibenzo[a,c]cyclohenten-3-yl ester (73, R⁴=Trifluoromethyl)

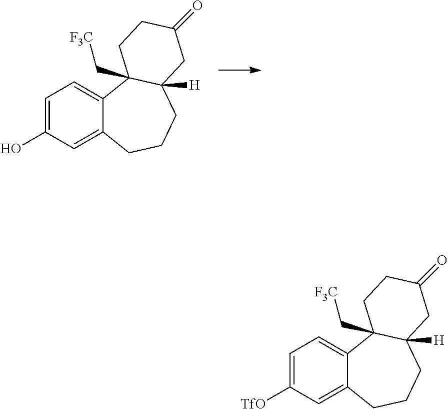

A mixture of (4aS,11bS)-9-hydroxy-11b-(2,2,2-trifluoro-ethyl)-1,2,4,4a,5,6,7,11b-octahydro-dibenzo[a,c]cyclohepten-3-one; compound with (4aR,11bR)-9-hydroxy-11b-(2,2,2-trifluoro-ethyl)-1,2,4,4a,5,6,7,11b-octahydro-dibenzo[a,c]cyclohepten-3-one (72, R⁴=Trifluoromethyl) (0.670 g, 2.14 mmol), 1,1,1-trifluoro-N-phenyl-N-(trifluoromethylsulfonyl)methanesulfonamide (0.766 g, 2.14 mmol), DIEA (0.749 mL, 4.29 mmol) and DCM (8 mL) was stirred at rt for about 18 h. The mixture was absorbed directly to silica gel (4 g), then purified on silica gel (25 g) eluting with a gradient of 5-40% EtOAc in heptane. Fractions containing product were combined and concentrated to yield trifluoro-methanesulfonic acid (7aS,11aS)-9-oxo-11a-(2,2,2-trifluoro-ethyl)-6,7,7a,8,9,10,11,11a-octahydro-5H-dibenzo[a,c]cyclohepten-3-yl ester; compound with trifluoro-methanesulfonic acid (7aR,11aR)-9-oxo-11a-(2,2,2-trifluoro-ethyl)-6,7,7a,8,9,10,11,11a-octahydro-5H-dibenzo[a,c]cyclohepten-3-yl ester (73, R⁴=Trifluoromethyl) (0.678 g, 71%) as a white solid. LC/MS, method 2, Rₜ=2.86 min, MS m/z 503 (M+OAc)⁻. ¹H NMR (400 MHz, DMSO-d₆) δ 7.62 (d, J=8.9 Hz, 1H), 7.36-7.25 (m, 2H), 3.50-3.33 (m, 1H), 3.14-3.02 (m, 1H), 2.96-2.82 (m, 2H), 2.61-2.42 (m, 1H), 2.42-2.30 (m, 2H), 2.30-2.18 (m, 1H), 2.09-1.89 (m, 3H), 1.76-1.66 (m, 1H), 1.65-1.55 (m, 1H), 1.52-1.39 (m, 1H), 1.31-1.21 (m, 1H).

### Step #7: (7aS,11aS)-9-Oxo-11a-(2,2,2-trifluoro-ethyl)-6,7,7a,8,9,10,11,11a-octahydro-5H-dibenzo[a,c]cycloheptene-3-carboxylic acid methyl ester; compound with (7aR,11aR)-9-oxo-11a-(2,2,2-trifluoro-ethyl)-6,7,7a,8,9,10,11,11a-octahydro-5H-dibenzo[a,c]cycloheptene-3-carboxylic acid methyl ester (74, R⁴=Trifluoromethyl)

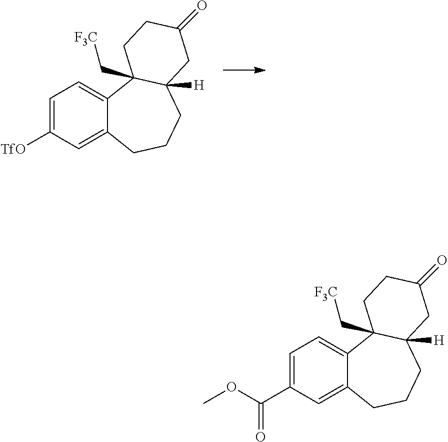

To trifluoro-methanesulfonic acid (7aS,11aS)-9-oxo-11a-(2,2,2-trifluoro-ethyl)-6,7,7a,8,9,10,11,11a-octahydro-5H-dibenzo[a,c]cyclohepten-3-yl ester; compound with trifluoro-methanesulfonic acid (7aR,11aR)-9-oxo-11a-(2,2,2-trifluoro-ethyl)-6,7,7a,8,9,10,11,11a-octahydro-5H-dibenzo[a,c]cyclohepten-3-yl ester (73, R⁴=Trifluoromethyl) (0.678 g, 1.53 mmol), Xantphos (0.088 g, 0.153 mmol) and Pd₂(dba)₃ (0.042 g, 0.046 mmol) was added DMF (6 mL). The mixture was flushed with N₂, then evacuated. CO gas was introduced via balloon and then TEA (0.425 mL, 3.05 mmol) and MeOH (0.370 mL, 9.15 mmol) were added. The mixture was heated under CO at about 60° C. for about 18 h. The reaction was cooled and concentrated under reduced pressure. The residue was purified on silica gel (12 g) eluting with a gradient of 5-50% EtOAc in heptane. Product fractions were combined and concentrated under reduced pressure to yield (7aS,11aS)-9-oxo-11a-(2,2,2-trifluoro-ethyl)-6,7,7a,8,9,10,11,11a-octahydro-5H-dibenzo[a,c]cycloheptene-3-carboxylic acid methyl ester; compound with (7aR,11aR)-9-oxo-11a-(2,2,2-trifluoro-ethyl)-6,7,7a,8,9,10,11,11a-octahydro-5H-dibenzo[a,c]cycloheptene-3-carboxylic acid methyl ester (74, R⁴=Trifluoromethyl) (0.320 g, 59%) as an oil. LC/MS, method 3, Rₜ=2.54 min, no parent mass. ¹H NMR (400 MHz, DMSO-d₆) δ 7.79-7.75 (m, 2H), 7.59 (d, J=8.1 Hz, 1H), 3.83 (s, 3H), 3.50-3.34 (m, 1H), 3.15-3.02 (m, 1H), 2.98-2.85 (m, 2H), 2.62-2.43 (m, 1H), 2.41-2.31 (m, 2H), 2.30-2.20 (m, 1H), 2.06-1.97 (m, 2H), 1.79-1.68 (m, 1H), 1.65-1.54 (m, 1H), 1.52-1.39 (m, 1H), 1.30-1.21 (m, 2H).

### Step #8: (7aS,11aS)-9-Oxo-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide; compound with (7aR,11aR)-9-oxo-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide (75, R⁴=Trifluoromethyl)

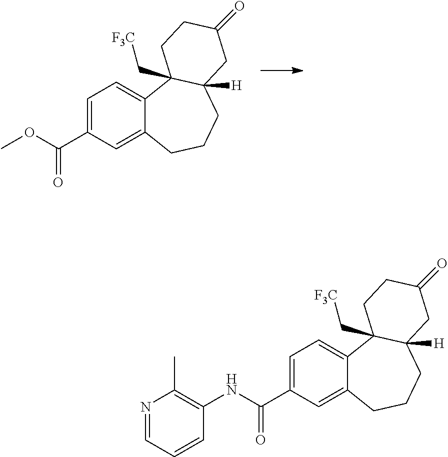

To (7aS,11aS)-9-oxo-11a-(2,2,2-trifluoro-ethyl)-6,7,7a,8,9,10,11,11a-octahydro-5H-dibenzo[a,c]cycloheptene-3-carboxylic acid methyl ester; compound with (7aR,11aR)-9-oxo-11a-(2,2,2-trifluoro-ethyl)-6,7,7a,8,9,10,11,11a-octahydro-5H-dibenzo[a,c]cycloheptene-3-carboxylic acid methyl ester (74, R⁴=Trifluoromethyl) (0.320 g, 0.903 mmol) was added LiOH (0.108 g, 4.52 mmol) in MeOH (2 mL) and water (2 mL). The mixture was heated to about 60° C. for about 1 h, then stirred at rt for about 18 h. The reaction was concentrated to remove MeOH, then 5 N aqueous HCl was added dropwise to pH ˜2. The solid was collected by filtration and rinsed with water to provide (7aS,11aS)-9-oxo-11a-(2,2,2-trifluoro-ethyl)-6,7,7a,8,9,10,11,11a-octahydro-5H-dibenzo[a,c]cycloheptene-3-carboxylic acid; compound with (7aR,11aR)-9-oxo-11a-(2,2,2-trifluoro-ethyl)-6,7,7a,8,9,10,11,11a-octahydro-5H-dibenzo[a,c]cycloheptene-3-carboxylic acid (0.278 g, 90%) as a white solid. LC/MS, method 3, Rₜ=1.99 min, MS m/z 339 (M−H)^{- 1}H NMR (400 MHz, DMSO-d₆) δ 12.86 (s, 1H), 7.78-7.71 (m, 2H), 7.56 (d, J=8.2 Hz, 1H), 3.50-3.34 (m, 1H), 3.08 (t, J=13.4 Hz, 1H), 2.99-2.88 (m, 2H), 2.61-2.52 (m, 1H), 2.41-2.19 (m, 4H), 2.06-1.89 (m, 3H), 1.71 (s, 1H), 1.64-1.58 (m, 1H), 1.56-1.45 (m, 1H). To (7aS,11aS)-9-oxo-11a-(2,2,2-trifluoro-ethyl)-6,7,7a,8,9,10,11,11a-octahydro-5H-dibenzo[a,c]cycloheptene-3-carboxylic acid; compound with (7aR,11aR)-9-oxo-11a-(2,2,2-trifluoro-ethyl)-6,7,7a,8,9,10,11,11a-octahydro-5H-dibenzo[a,c]cycloheptene-3-carboxylic acid (0.357 g, 1.05 mmol) was added DIEA (0.256 mL, 1.47 mmol) and THF (5 mL) and the mixture was stirred for about 5 min. BTFFH (0.348 g, 1.10 mmol) was added and the mixture stirred about 15 min. 2-Methylpyridin-3-amine (0.170 g, 1.57 mmol) was added and the mixture was heated to about 60° C. for about 5 h. Additional DIEA (0.100 mL, 0.574 mmol) and 2-methylpyridin-3-amine (0.030 g, 0.278 mmol) were added and the mixture was stirred at rt for about 72 h. The mixture was concentrated in vacuo and then purified on silica gel (12 g), eluting with a gradient of 50-100% EtOAc in heptane to provide (7aS,11aS)-9-oxo-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide; compound with (7aR,11aR)-9-oxo-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide (75, R⁴=Trifluoromethyl) (0.452 g, 100%). LC/MS, method 3, Rₜ=2.06 min, MS m/z 429 (M−H)⁻.

### Step #10: (+/−) Compound 76 (R⁴=Trifluoromethyl)

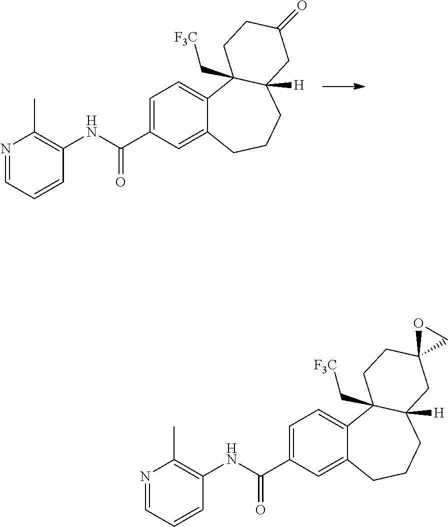

DMSO (2 mL) was added to NaH (60% dispersion in mineral oil, 0.084 g, 2.1 mmol) under N₂ and the mixture was heated at about 60° C. for about 1 h. The reaction was cooled to rt, diluted with THF (2 mL) and then cooled to about 0° C. Trimethylsulfoxonium iodide (0.462 g, 2.10 mmol) was added then the reaction was stirred for about 10 min. A suspension of (7aS,11aS)-9-oxo-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide; compound with (7aR,11aR)-9-oxo-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide (75, R⁴=Trifluoromethyl) (0.452 g, 1.05 mmol) in THF (2 mL) was added and the reaction was allowed to warm to rt and was stirred for about 18 h. THF was removed under reduced pressure and the residue taken up in EtOAc (20 mL). The resulting solids were collected by filtration and washed with water (20 mL) to provide (+/−) Compound 76 (R⁴=Trifluoromethyl) (0.467 g, 100%). LC/MS, method 2, Rₜ=2.31 min, MS m/z 391 (M+H)⁺.

### Step #11: (7aS,9S,11aS)-9-Hydroxy-9-isobutyl-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-9-hydroxy-9-isobutyl-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide (77, R⁴=Trifluoromethyl, R⁵=Isopropyl)

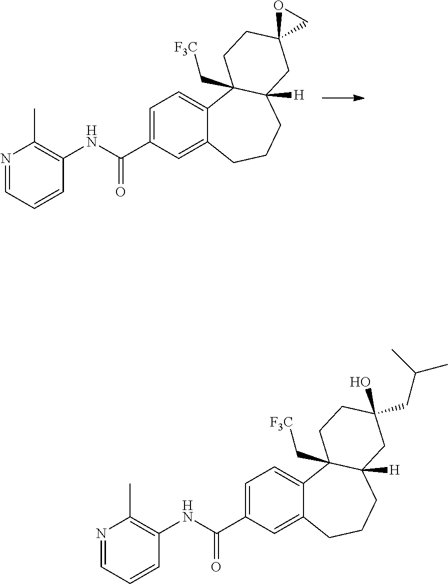

To (+/−) Compound 76 (R⁴=Trifluoromethyl) (0.159 g, 0.358 mmol) in THF (3 mL) under N₂ was added copper(I)iodide (0.0068 g, 0.036 mmol) and the mixture was cooled to about 0° C. for about 5 min. Isopropylmagnesium bromide (2.9 M in 2-methyltetrahydrofuran, 0.200 mL, 0.580 mmol) was then added dropwise and the mixture stirred for about 18 h. The reaction was quenched with saturated aqueous NH₄Cl (10 mL) and extracted with EtOAc (20 mL). The organic layer was dried over MgSO₄, filtered and concentrated under reduced pressure. The residue was purified on silica gel (4 g) eluting with a gradient of 50-100% EtOAc in heptane. Product containing fractions were combined and concentrated under reduced pressure. The residue was disolved in MeOH (0.20 mL) and water (5 mL) was added. The resulting precipitate was collected by filtration, washed with water (2.0 mL) and dried under reduced pressure to yield (7aS,9S,11aS)-9-hydroxy-9-isobutyl-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-9-hydroxy-9-isobutyl-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide (77, R⁴=Trifluoromethyl, R⁵=isopropyl) (0.012 g, 7%). LC/MS, method 2, Rₜ=2.40 min, MS m/z 489 (M+H)⁺. ¹H NMR (400 MHz, DMSO-d₆) δ 9.99 (s, 1H), 8.33 (dd, J=4.8, 1.6 Hz, 1H), 7.79-7.69 (m, 3H), 7.49 (d, J=8.4 Hz, 1H), 7.27 (dd, J=7.9, 4.8 Hz, 1H), 3.96 (s, 1H), 3.26-3.10 (m, 1H), 3.07-2.96 (m, 1H), 2.94-2.86 (m, 1H), 2.58-2.49 (m, 1H), 2.44 (s, 3H), 2.44-2.22 (m, 2H), 2.02-1.85 (m, 1H), 1.77-1.65 (m, 2H), 1.61-1.39 (m, 4H), 1.26-1.01 (m, 5H), 0.82 (d, J=6.6 Hz, 6H).

### Example 64

### (7aS,9R,11aR)-9-Cyanomethyl-11a-ethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aS)-9-cyanomethyl-11a-ethyl-9-hydroxy-6,7,7a,8,9,10,11,11a-octahydro-5H-dibenzo[a,c]cyclo heptene-3-carboxylic acid (2-methyl-pyridin-3-yl)-amide (77, R⁴=Methyl, R⁵=Cyano)

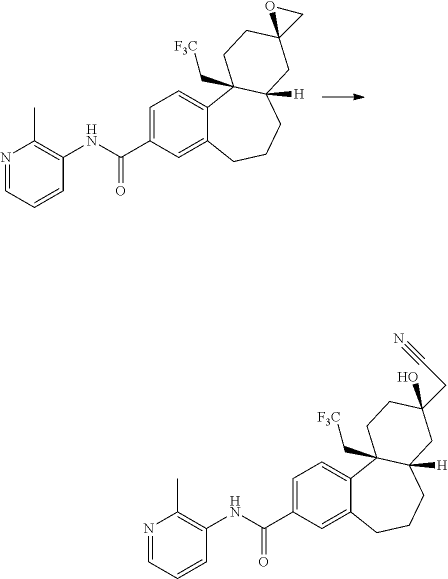

To a suspension of (+/−) Compound 76 (R⁴=Methyl) (0.060 g, 0.15 mmol) in toluene (2 mL) under nitrogen, a solution of 1 M diethyl aluminumcyanide (0.92 mL, 0.92 mmol) was added and the resulting heterogeneous mixture was stirred for about 16 h at rt. The mixture was treated with saturated aqueous sodium potassium tartrate (1 mL) and EtOAc (1 mL) and stirred for about 15 min. The layers were separated and the aqueous layer was extracted with EtOAc (10 mL). The combined organic layers were dried over MgSO₄, filtered and concentrated under reducd pressure. The residue was purified on silica gel (4 g) eluting with a gradient of 10-100% EtOAc in heptane to provide (7aS,9R,11aR)-9-cyanomethyl-11a-ethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aS)-9-cyanomethyl-11a-ethyl-9-hydroxy-6,7,7a,8,9,10,11,11a-octahydro-5H-dibenzo[a,c]cycloheptene-3-carboxylic acid (2-methyl-pyridin-3-yl)-amide (77, R⁴=Methyl, R⁵=Cyano) (0.021 g, 33%). LC/MS, method 2, Rₜ=1.87 min, MS m/z 418 (M+H)⁺¹H NMR (400 MHz, DMSO-d₆) δ 9.95 (s, 1H), 8.33 (dd, J=4.8, 1.6 Hz, 1H), 7.79-7.68 (m, 3H), 7.38 (d, J=8.3 Hz, 1H), 7.27 (dd, J=8.1, 4.7 Hz, 1H), 4.94 (s, 1H), 3.32 (s, 2H), 3.05-2.95 (m, 1H), 2.93-2.83 (m, 1H), 2.43 (s, 3H), 2.39-2.19 (m, 3H), 2.12-2.01 (m, 1H), 1.78-1.37 (m, 7H), 1.29-1.21 (m, 2H), 0.61 (t, J=7.3 Hz, 3H).

Additional examples, prepared in a manner similar to the preparation of Example #64 are listed in Table 3

TABLE 3

| | | | | | | Chiral |
|---|---|---|---|---|---|---|
| | | | | | LC/MS | method/ |
| | | | | LC/MS | RT/ | Order of |
| Ex.# | Epoxide | Reagent | Product | method | MH+ | elution |
| 65 | Compound | Diethyl | Compound 77 | 2 | 2.05/ | NA |
| | 28 (R² = | aluminumcyanide | (7aS,9R,11aS); | | 480 | |
| | Benzyl) | | compound with | | | |
| | | | (7aR, | | | |
| | | | 9S,11aR), (R⁴ = | | | |
| | | | Phenyl, R⁵ = | | | |
| | | | Cyano) | | | |
| 66 | Compound | Diethyl | Compound 77 | 2 | 2.05/ | 7/First |
| | 28 (R² = | aluminumcyanide | (7aS,9R,11aS), (R⁴ = | | 480 | |
| | Benzyl) | | Phenyl, R⁵ = | | | |
| | | | Cyano) | | | |
| 67 | Compound | Diethyl | Compound 77 | 2 | 2.05/ | 7/Second |
| | 28 (R² = | aluminumcyanide | (7aR,9S,11aR), (R⁴ = | | 480 | |
| | Benzyl) | | Phenyl, R⁵ = | | | |
| | | | Cyano) | | | |

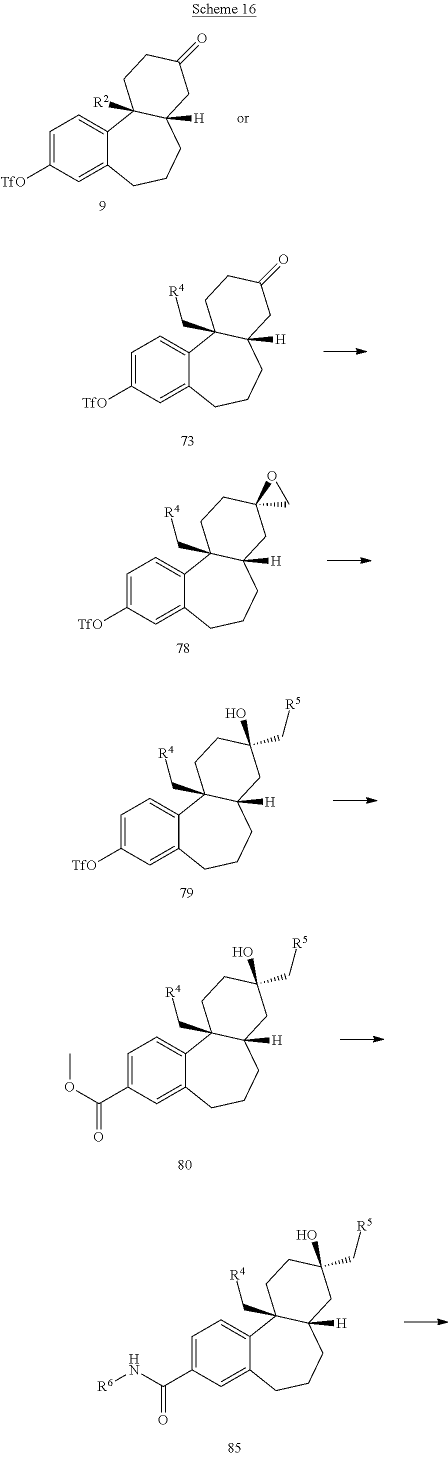

### Example #68

### (7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2,4-dimethyl-pyrimidin-5-yl)-amide (85, R⁴=Phenyl, R⁵=Methyl, R⁶=2,4-Pyrimidin-5-yl)

### Step 1: (+/−) Compound 78 (R⁴=Phenyl)

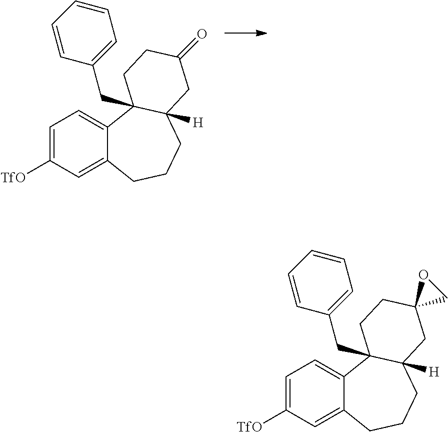

A 250 mL 3 necked round bottom flask equipped with a thermometer, septum, nitrogen line and stir bar was charged with DMSO (50 mL) and sodium hydride, 60% dispersion in mineral oil (0.707 g, 17.7 mmol). The mixture was warmed to an internal temperature of about 60° C. for about 30 min. The mixture was cooled to rt then trimethylsulfoxonium iodide (3.89 g, 17.7 mmol) was added. The mixture was stirred for about 10 min then cooled to about −10° C. The mixture was diluted with THF (50 mL) and then the trifluoro-methanesulfonic acid (7aS,11aS)-11a-benzyl-9-oxo-6,7,7a,8,9,10,11,11a-octahydro-5H-dibenzo[a,c]cyclohepten-3-yl ester; compound with trifluoro-methanesulfonic acid (7aR,11aR)-11a-benzyl-9-oxo-6,7,7a,8,9,10,11,11a-octahydro-5H-dibenzo[a,c]cyclohepten-3-yl ester (9, R²=Benzyl) (4.00 g, 8.84 mmol) was added. The mixture was stirred at about −10° C. for about 15 min then allowed to warm to about 10° C. over about 30 min. The mixture was stirred for about 1 h. Water (250 mL) was added then the mixture was extracted with EtOAc (100 mL, then 50 mL). The combined organics were extracted with water (250 mL) then saturated NaHCO₃ (˜40 mL) then saturated aqueous NaCl (˜50 mL). The organic solution was dried over MgSO₄, filtered, and concentrated under reduced pressure. The thick oil was dissolved in a minimum of DCM then the material was purified on silica gel (80 g) eluting with a gradient of 0-50% EtOAc in heptane. The fractions containing product were combined and concentrated under reduced pressure to give (+1-) Compound (78, R⁴=Phenyl) (2.39 g, 58%). LC/MS, method 3, Rₜ=3.53 min, MS m/z 525 (M+OAc)⁻. ¹H NMR (400 MHz, DMSO-d₆) δ 7.35 (d, J=2.6 Hz, 1H), 7.10-7.00 (m, 4H), 6.86 (d, J=8.3 Hz, 1H), 6.53 (d, J=6.9 Hz, 2H), 3.58 (d, J=13.0 Hz, 1H), 3.28-3.18 (m, 1H), 3.04-2.96 (m, 1H), 2.63 (d, J=13.1 Hz, 1H), 2.53-2.49 (m, 2H), 2.46-2.38 (m, 1H), 2.33-2.23 (m, 1H), 2.12-1.89 (m, 2H), 1.83-1.62 (m, 4H), 1.57-1.43 (m, 1H), 1.15-1.07 (m, 1H), 0.92-0.80 (m, 1H).

### Step 2: Trifluoro-methanesulfonic acid (7aS,9R,11aS)-11a-benzyl-9-ethyl-9-hydroxy-6,7,7a,8,9,10,11,11a-octahydro-5H-dibenzo[a,c]cyclohepten-3-yl ester; compound with trifluoro-methanesulfonic acid (7aR,9S,11aR)-11a-benzyl-9-ethyl-9-hydroxy-6,7,7a,8,9,10,11,11a-octahydro-5H-dibenzo[a,c]cyclohepten-3-yl ester (79, R⁴=Phenyl, R⁵=Methyl)

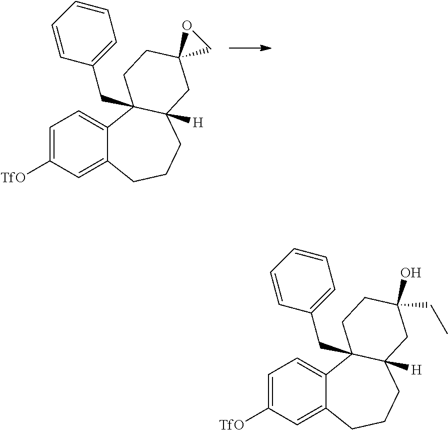

A 3 necked round bottom flask with stir bar, septum, nitrogen line and thermometer was charged with THF (50 mL) and copper(I)iodide (1.76 g, 9.23 mmol). The mixture was cooled to an internal temperature of about −40° C. then methylmagnesium bromide (3M solution in Et₂O, 5.64 mL, 16.9 mmol) was while maintaining the reaction temperature between about −30 to −40° C. After complete addition, the mixture was stirred for about 30 min allowing the temperature to rise to about 0° C. After about 15 min at about 0° C., the mixture was cooled to about −40° C. then (+/−) Compound 78 (R⁴=Phenyl) (3.16 g, 6.77 mmol) in THF (50 mL) was added keeping the internal temperature between about −30 to −40° C. After complete addition of the epoxide, the mixture was stirred at about −40° C. After about 15 min the temperature of the mixture was allowed to rise slowly to about 0° C. over about 2 h. Another portion of methylmagnesium bromide (3 M solution in Et₂O, 2.26 mL, 6.77 mmol) was added then the mixture was stirred at about 0° C. for about 30 min. The reaction was quenched with saturated aqueous NH₄Cl (50 mL) then stirred for about 5 min then let stand for about 18 h. The mixture was diluted with Et₂O (100 mL) and water (100 mL). The layers were separated then the aqueous layer was extracted with Et₂O (100 mL). The combined organics were washed with saturated aqueous NaCl (50 mL), dried over MgSO₄, filtered and concentrated under reduced pressure. The residue was purified on silica gel (80 g) eluting with a gradient 0-50% EtOAc in heptane. The fractions containing product were concentrated to give trifluoro-methanesulfonic acid (7aS,9R,11aS)-11a-benzyl-9-ethyl-9-hydroxy-6,7,7a,8,9,10,11,11a-octahydro-5H-dibenzo[a,c]cyclohepten-3-yl ester; compound with trifluoro-methanesulfonic acid (7aR,9S,11aR)-11a-benzyl-9-ethyl-9-hydroxy-6,7,7a,8,9,10,11,11a-octahydro-5H-dibenzo[a,c]cyclohepten-3-yl ester (79, R⁴=Phenyl, R⁵=Methyl) (3.16 g, 97%); LC/MS, method 2, Rₜ=3.38 min, MS m/z 541 (M+OAc)⁻. ¹H NMR (400 MHz, DMSO-d₆) δ 7.30 (d, J=2.9 Hz, 1H), 7.06-6.98 (m, 4H), 6.77 (d, J=9.0 Hz, 1H), 6.50-6.45 (m, 2H), 3.91 (s, 1H), 3.54 (d, J=13.0 Hz, 1H), 3.24-3.17 (m, 1H), 3.03-2.96 (m, 1H), 2.42 (d, J=13.0 Hz, 1H), 1.81-1.73 (m, 3H), 1.64-1.35 (m, 3H), 1.30-1.04 (m, 7H), 0.69 (t, J=7.4 Hz, 3H)

### Step 3: (7aS,9R,11aS)-11a-Benzyl-9-ethyl-9-hydroxy-6,7,7a,8,9,10,11,11a-octahydro-5H-dibenzo[a,c]cycloheptene-3-carboxylic acid methyl ester; compound with (7aR,9S,11aR)-11a-benzyl-9-ethyl-9-hydroxy-6,7,7a,8,9,10,11,11a-octahydro-5H-dibenzo[a,c]cycloheptene-3-carboxylic acid methyl ester (80, R⁴=Phenyl, R⁵=Methyl)

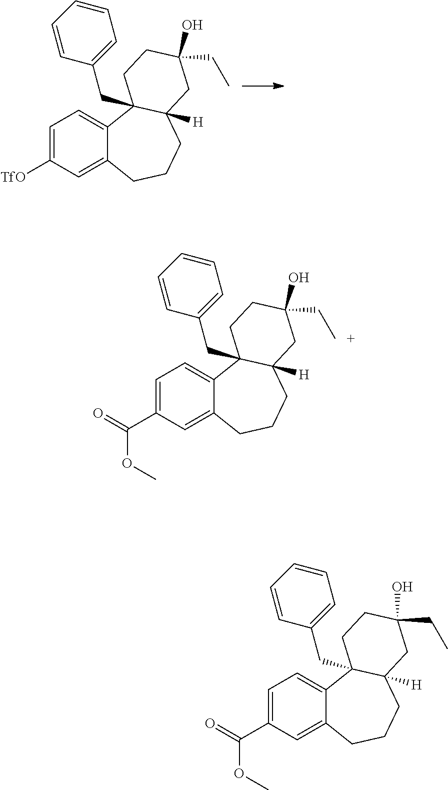

A 500 mL round bottom flask containing the trifluoro-methanesulfonic acid (7aS,9R,11aS)-11a-benzyl-9-ethyl-9-hydroxy-6,7,7a,8,9,10,11,11a-octahydro-5H-dibenzo[a,c]cyclohepten-3-yl ester; compound with trifluoro-methanesulfonic acid (7aR,9S,11aR)-11a-benzyl-9-ethyl-9-hydroxy-6,7,7a,8,9,10,11,11a-octahydro-5H-dibenzo[a,c]cyclohepten-3-yl ester (79, R⁴=Phenyl, R⁵=Methyl) (3.16 g, 6.55 mmol) equipped with a stir bar, a 3 way stopcock connected to a vacuum line and a carbon monoxide filled balloon, was charged with DMF (50 mL). The mixture was stirred under vacuum (˜15 torr) for about 15 min then the flask was filled with carbon monoxide and charged with Xantphos (0.379 g, 0.655 mmol), Pd₂(dba)₃ (0.180 g, 0.196 mmol), MeOH (3.2 mL, 79 mmol) and TEA (3.7 mL, 26 mmol). The flask was evacuated then filled with carbon monoxide. This was repeated two more times then the mixture was heated in an oil bath at about 90° C. with rapid stirring for about 22 h. The mixture was cooled and concentrated under reduced pressure. The mixture was treated with MeOH (30 mL) then concentrated under reduced pressure. The material was partitioned between EtOAc (50 mL) and water (50 mL). The organic solution was extracted with saturated aqueous NaCl (30 mL) then dried over MgSO₄, filtered and concentrated under reduced pressure. The material was purified on silica gel (80 g) eluting with a gradient of 0-50% EtOAc in heptane. The fractions containing product were concentrated under reduced pressure to give (7aS,9R,11aS)-11a-benzyl-9-ethyl-9-hydroxy-6,7,7a,8,9,10,11,11a-octahydro-5H-dibenzo[a,c]cycloheptene-3-carboxylic acid methyl ester; compound with (7aR,9S,11aR)-11a-benzyl-9-ethyl-9-hydroxy-6,7,7a,8,9,10,11,11a-octahydro-5H-dibenzo[a,c]cycloheptene-3-carboxylic acid methyl ester (80, R⁴=Phenyl, R⁵=Methyl) (1.63 g, 63%); LC/MS, method 3, Rₜ=3.07 min, MS m/z 451 (M+OAc)⁻. ¹H NMR (400 MHz, DMSO-d₆) δ 7.76 (d, J=2.1 Hz, 1H), 7.50 (dd, J=8.2, 2.0 Hz, 1H), 7.09-7.00 (m, 3H), 6.78 (d, J=8.4 Hz, 1H), 6.54-6.51 (m, 2H), 3.89 (s, 1H), 3.82 (s, 3H), 3.57 (d, J=12.9 Hz, 1H), 3.26-3.19 (m, 1H), 3.03-2.98 (m, 1H), 2.57 (d, J=12.9 Hz, 1H), 2.45-2.39 (m, 2H), 1.90-1.77 (m, 3H), 1.60-1.22 (m, 4H), 1.17-1.00 (m, 4H), 0.68 (t, J=7.4 Hz, 3H).

The enantiomers were separated by chiral preparative chromatography (The gradient was 1-3% A in 17 min (20 mL/min flow rate). Mobile phase A was EtOH (200 proof), mobile phase B was HPLC grade heptane with 0.1% DEA added. The column used for the chromatography was a Daicel IA, 20×250 mm column (5 μm particles). Detection methods were evaporative light scattering (ELSD) detection as well as optical rotation to provide (7aS,9R,11aS)-11a-benzyl-9-ethyl-9-hydroxy-6,7,7a,8,9,10,11,11a-octahydro-5H-dibenzo[a,c]cycloheptene-3-carboxylic acid methyl ester (80, R⁴=Phenyl, R⁵=Methyl) (0.725 g, 29%, negative rotation) and (7aR,9S,11aR)-11a-benzyl-9-ethyl-9-hydroxy-6,7,7a,8,9,10,11,11a-octahydro-5H-dibenzo[a,c]cycloheptene-3-carboxylic acid methyl ester (80, R⁴=Phenyl, R⁵=Methyl) (0.696 g, 27%, positive rotation). NMR and LC/MS data for single isomers was essentially identical to the racemic mixture.

### Step 4: (7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2,4-dimethyl-pyrimidin-5-yl)-amide (85, R⁴=Phenyl, R⁵=Methyl, R⁶=2,4-Pyrimidin-5-yl)

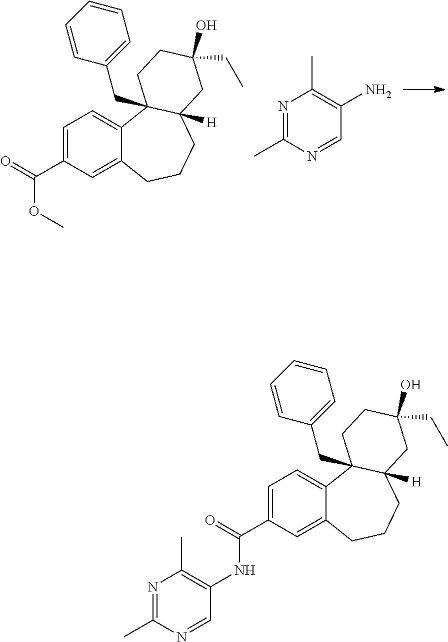

A mixture of (7aS,9R,11aS)-11a-benzyl-9-ethyl-9-hydroxy-6,7,7a,8,9,10,11,11a-octahydro-5H-dibenzo[a,c]cycloheptene-3-carboxylic acid methyl ester (80, R⁴=Phenyl, R⁵=Methyl) (75 mg, 0.19 mmol) in toluene (2 mL) was treated with 2,4-dimethylpyrimidin-5-amine (35 mg, 0.29 mmol) then LiHMDS (1 M solution in THF, 0.57 mL, 0.57 mmol). After about 1 h, the reaction was diluted with EtOAc (25 mL) then washed with saturated aqueous NH₄Cl (10 mL) and water (5 mL). The organic layer was dried over MgSO₄, filtered and concentrated under reduced pressure. The material was purified on silica gel (12 g) eluting with a gradient of 0-10% MeOH in DCM. The fractions with desired material were concentrated to dryness then the material was dissolved in MeOH (5 mL). The solution was diluted with water (5 mL) to form a milky mixture. The MeOH was removed under reduced pressure and the resulting solids were collected by filtration and washed with water (˜5 mL). The material was dried under vacuum at about 65° C. to give (7aS,9R,11aS)-11a-benzyl-9-ethyl-9-hydroxy-6,7,7a,8,9,10,11,11a-octahydro-5H-dibenzo[a,c]cycloheptene-3-carboxylic acid (2,4-dimethyl-pyrimidin-5-yl)-amide (85, R⁴=Phenyl, R⁵=Methyl, R⁶=2,4-Pyrimidin-5-yl) (45 mg, 49%); LC/MS method 2, Rₜ=2.26 min, MS m/z 484 (M+H)⁺; ¹H NMR (400 MHz, DMSO-d₆) δ 10.06 (s, 1H), 8.56 (s, 1H), 7.81 (d, J=2.1 Hz, 1H), 7.57-7.54 (m, 1H), 7.08-7.04 (m, 3H), 6.82 (d, J=8.4 Hz, 1H), 6.60-6.57 (m, 2H), 3.89 (s, 1H), 3.59 (d, J=12.9 Hz, 1H), 3.28 (d, J=12.9 Hz, 1H), 3.05-2.99 (m, 1H), 2.62-2.52 (m, 4H), 2.48-2.41 (m, 2H), 2.39 (s, 3H), 1.86-1.94 (m, 1H), 1.74-1.84 (m, 2H), 1.55-1.57 (m, 2H), 1.41-1.47 (m, 1H), 1.28-1.36 (m, 1H), 1.04-1.17 (m, 4H), 0.71 (t, J=7.4 Hz, 3H)

Additional examples, prepared in a manner similar to the preparation of Example #68 are listed in Table 4.

TABLE 4

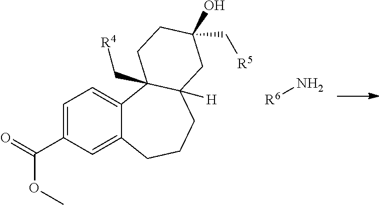

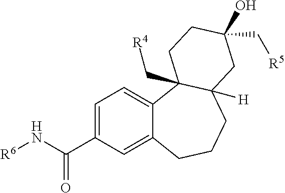

| | | | LC/MS | |
|---|---|---|---|---|
| | | | method/ | m/z ESI+ |
| Ex. # | Amine | Product | Rₜ min | (M + H)⁺ |
| 69 | 3,5-Dimethylpyrazin-2-amine | Compound 85 (7aS, 9R, 11aS) | 2/2.42 | 484 |
| | [Maybridge] | (R⁴ = Phenyl, R⁵ = Methyl, | | |
| | | R⁶ = 3,5-Dimethylpyrazin-2-yl) | | |
| 70 | 3-Methylpyridin-4-amine | Compound 85 (7aS, 9R, 11aS) | 2/2.49 | 469 |
| | [SynChem] | (R⁴ = Phenyl, R⁵ = Methyl, | | |
| | | R⁶ = 3-Methylpyridin-4-yl) | | |
| 71 | 4-Methylpyridin-3-amine | Compound 85 (7aS, 9R, 11aS) | 2/2.36 | 469 |
| | [Asymchem] | (R⁴ = Phenyl, R⁵ = Methyl, | | |
| | | R⁶ = 4-Methylpyridin-3-yl) | | |
| 72 | 2,6-Dimethylpyridin-3-amine | Compound 85 (7aS, 9R, 11aS) | 2/2.38 | 483 |
| | [Lancaster] | (R⁴ = Phenyl, R⁵ = Methyl, | | |
| | | R⁶ = 2,6-Dimethylpyridin-3-yl) | | |
| 73 | 3-Methylpyridin-2-amine | Compound 85 (7aS, 9R, 11aS) | 2/2.50 | 469 |
| | | (R⁴ = Phenyl, R⁵ = Methyl, | | |
| | | R⁶ = 3-Methylpyridin-2-yl) | | |
| 74 | 1,3,4-Thiadiazol-2-amine | Compound 85 (7aS, 9R, 11aS) | 2/2.43 | 462 |
| | | (R⁴ = Phenyl, R⁵ = Methyl, | | |
| | | R⁶ = 1,3,4-Thiadiazol-3-yl | | |
| 75 | 1-Methyl-1H-pyrazol-5-amine | Compound 85 (7aS, 9R, 11aS) | 2/2.29 | 458 |
| | [Combiblocks] | (R⁴ = Phenyl, R⁵ = Methyl, | | |
| | | R⁶ = 1-Methyl-1H-pyrazol-5-yl | | |
| 76 | 1,3-Dimethyl-1H-pyrazol-5- | Compound 85 (7aS, 9R, 11aS) | 2/2.35 | 472 |
| | amine | (R⁴ = Phenyl, R⁵ = Methyl, | | |
| | | R⁶ = 1,3-Dimethyl-1H-pyrazol-5-yl | | |
| 77 | 2,4-Dimethylpyrimidin-5-amine | Compound 85 (7aR, 9S, 11aR) | 2/2.26 | 484 |
| | [Tyger] | (R⁴ = Phenyl, R⁵ = Methyl, | | |
| | | R⁶ = 1,3-Dimethyl-1H-pyrazol-5-yl | | |
| 78 | 1-Methyl-1H-tetrazol-5-amine | Compound 85 (7aS, 9R, 11aS)) | 2/2.35 | 460 |
| | | (R⁴ = Phenyl, R⁵ = Methyl, | | |
| | | R⁶ = 1-Methyl-1H-tetrazol-5-yl) | | |
| 79 | 5-Methyl-1H-pyrazol-3-amine | Compound 85 (7aS, 9R, 11aS)) | 2/2.30 | 458 |
| | [CombiBlocks] | (R⁴ = Phenyl, R⁵ = Methyl, | | |
| | | R⁶ = 5-Methyl-1H-pyrazol-3-yl) | | |
| 80 | 1-Methyl-1H-pyrazol-5-amine | Compound 85 (7aR, 9R, 11aS)) | 2/2.20 | 498 |
| | [Combiblocks] | (R⁴ = Phenyl, R⁵ = Trifluoromethyl, | | |
| | | R⁶ = 5-Methyl-1H-pyrazol-3-yl) | | |
| 81 | 3-Methylpyridin-4-amine | Compound 85 (7aR, 9R, 11aS) | 2/2.34 | 509 |
| | [SynChem] | (R⁴ = Phenyl, R⁵ = Trifluoromethyl, | | |
| | | R⁶ = 3-Methylpyridin-4-yl) | | |
| 82 | 1-Methyl-1H-pyrazol-5-amine | Compound 85 (7aS, 9S, 11aR)) | 2/2.20 | 498 |
| | [Combiblocks] | (R⁴ = Phenyl, R⁵ = Trifluoromethyl, | | |
| | | R⁶ = 5-Methyl-1H-pyrazol-3-yl) | | |

### Example #83

### (7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2-methyl-pyridin-3-ylmethyl)-amide (85, R⁴=Phenyl, R⁵=Methyl, R⁶=3-(2-Methyl-pyridin-3-ylmethyl)

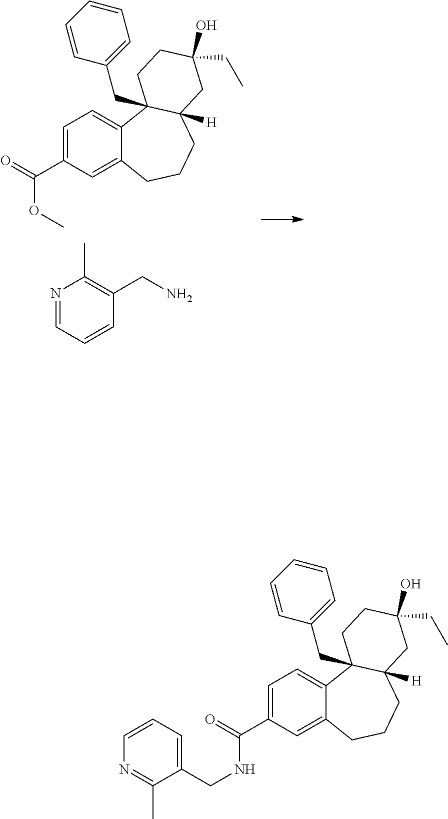

(7aS,9R,11aS)-11a-Benzyl-9-ethyl-9-hydroxy-6,7,7a,8,9,10,11,11a-octahydro-5H-dibenzo[a,c]cycloheptene-3-carboxylic acid methyl ester (80, R⁴=Phenyl, R⁵=Methyl) (85 mg, 0.22 mmol) in a mixture of 1,4-dioxane (4 mL) and water (1 mL) was treated with LiOH (42 mg, 1.7 mmol). The mixture was heated to about 80° C. for about 1 h. The mixture was cooled to rt then partitioned between EtOAc (25 mL) and 1N aqueous HCl (˜10 mL). The layers were separated then the organic solution was dried over MgSO₄, filtered and concentrated under reduced pressure to give (7aS,9R,11aS)-11a-benzyl-9-ethyl-9-hydroxy-6,7,7a,8,9,10,11,11a-octahydro-5H-dibenzo[a,c]cycloheptene-3-carboxylic acid (80 mg, 98%); LC/MS, method 2, Rₜ=2.35 min, MS m/z 377 (M−H)⁻. ¹H NMR (400 MHz, DMSO-d₆) δ 12.71 (s, 1H), 7.74 (s, 1H), 7.47 (d, J=8.3 Hz, 1H), 7.11-6.99 (m, 3H), 6.76 (d, J=8.4 Hz, 1H), 6.53 (dd, J=7.4, 1.8 Hz, 2H), 3.87 (s, 1H), 3.56 (d, J=12.8 Hz, 1H), 3.28-3.19 (m, 1H), 3.02-3.19 (m, 1H), 2.57 (d, J=12.8 Hz, 1H), 2.42 (m, 2H), 1.93-1.70 (m, 3H), 1.68-0.97 (m, 8H), 0.69 (t, J=7.4 Hz, 3H).

A mixture of the (7aS,9R,11aS)-11a-benzyl-9-ethyl-9-hydroxy-6,7,7a,8,9,10,11,11a-octahydro-5H-dibenzo[a,c]cycloheptene-3-carboxylic acid (40 mg, 0.11 mmol) was dissolved in DMF (2 mL) then treated with COMU (54 mg, 0.13 mmol), (2-methylpyridin-3-yl)methanamine [Archiv der Pharmazie (Weinheim, Germany), 1975, vol. 308, p. 969] (15 mg, 0.13 mmol) and DIEA (0.055 mL, 0.32 mmol). After about 5 min, the solvents were removed under reduced pressure then the material was partitioned between EtOAc (20 mL) and water (10 mL). The layers were separated then the organic solution was washed with saturated aqueous NaCl (10 mL), dried over MgSO₄, filtered and the filtrate concentrated under reduced pressure. The material was purified on silica gel (4 g) eluting with a gradient of 0-10% MeOH in DCM. The fractions containing product were concentrated under reduced pressure then dissolved in about 1 mL MeOH. Water (˜15 mL) was added. The mixture was concentrated under reduced pressure to remove the MeOH then the material was collected by filtration and dried under vacuum at about 65° C. to give (7aS,9R,11aS)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-ylmethyl)-amide (85, R⁴=Phenyl, R⁵=Methyl, R⁶=3-(2-Methyl-pyridin-3-ylmethyl), (31.6 mg, 62.0%); LC/MS, method 2, Rₜ=2.26 min; MS m/z: 483 (M+H)⁺; ¹H NMR (400 MHz, DMSO-d₆) δ 8.87 (t, J=5.7 Hz, 1H), 8.32 (dd, J=4.8, 1.7 Hz, 1H), 7.71 (d, J=2.1 Hz, 1H), 7.60 (dd, J=7.7, 1.8 Hz, 1H), 7.44 (dd, J=8.2, 2.1 Hz, 1H), 7.19 (dd, J=7.7, 4.8 Hz, 1H), 7.08-7.01 (m, 3H), 6.74 (d, J=8.4 Hz, 1H), 6.55 (d, J=1.9 Hz, 1H), 6.54 (d, J=2.8 Hz, 1H), 4.44 (d, J=5.7 Hz, 2H), 3.87 (s, 1H), 3.55 (d, J=12.9 Hz, 1H), 3.00-2.94 (m, 1H), 2.53 (s, 3H), 1.89-1.70 (m, 3H), 1.64-1.38 (m, 3H), 1.34-1.02 (m, 8H), 0.85 (m, 1H), 0.69 (t, J=7.4 Hz, 3H).

Additional examples, prepared in a manner similar to the preparation of Example #83 are listed in Table 5.

TABLE 5

| | | | LC/MS | |
|---|---|---|---|---|
| Ex. | | | method/ | m/z ESI+ |
| # | Amine | Product | Rₜ min | (M + H)⁺ |
| 84 | 2-Morpholino- | Compound 85 (7aS,9R,11aS) | 2/1.91 | 491 |
| | ethanamine | (R⁴ = Phenyl | | |
| | | R⁵ = Methyl | | |
| | | R⁶ = 2-Morpholinoethyl) | | |
| 85 | 2-Amino-1- | Compound 85 (7aS,9R,11aS) | 2/2.48 | 474 |
| | methyl-1H- | (R⁴ = Phenyl, | | |
| | imidazol- | R⁵ = Methyl, | | |
| | 4(5H)-one | R⁶ = 3-(1-Methyl-4-oxo-4,5- | | |
| | | dihydro-1H-imidazol-2-yl)) | | |
| 86 | 1-Ethyl-1H- | Compound 85 (7aS,9R,11aS) | 2/2.39 | 472 |
| | pyrazol-5- | (R⁴ = Phenyl, | | |
| | amine | R⁵ = Methyl, | | |
| | | R⁶ = 1-Ethyl-1H-pyrazol-5-yl) | | |

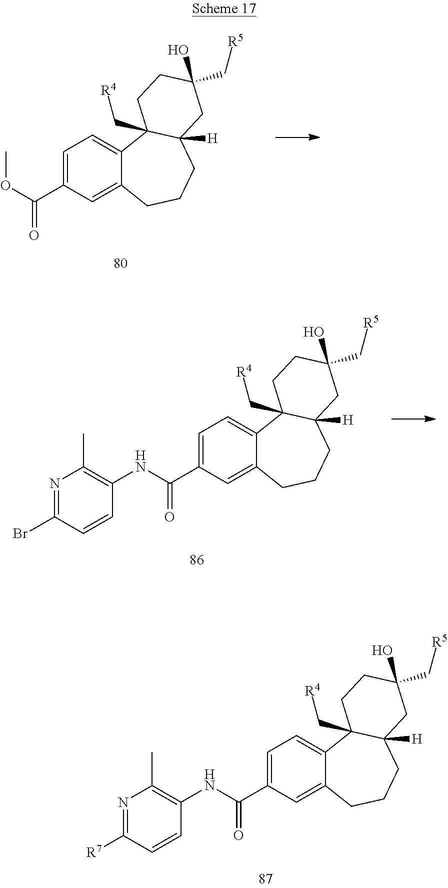

### Example #87

### (7aS,9R,11aS)-11a-Benzyl-9-ethyl-9-hydroxy-6,7,7a,8,9,10,11,11a-octahydro-5H-dibenzo[a,c]cycloheptene-3-carboxylic acid [2-methyl-6-(2H-pyra zol-3-yl)-pyridin-3-yl]-amide (87, R⁴=Phenyl, R⁵=Methyl, R⁷=(2H-pyrazol-3-yl)

### Step 1: (7aS,9R,11aS)-11a-Benzyl-9-ethyl-9-hydroxy-6,7,7a,8,9,10,11,11a-octahydro-5H-dibenzo[a,c]cycloheptene-3-carboxylic acid (6-bromo-2-methyl-pyridin-3-yl)-amide (86, R⁴=Phenyl, R⁵=Methyl)

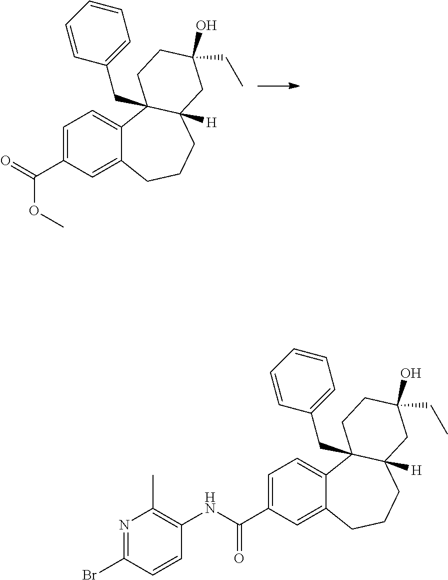

(7aS,9R,11aS)-11a-Benzyl-9-ethyl-9-hydroxy-6,7,7a,8,9,10,11,11a-octahydro-5H-dibenzo[a,c]cycloheptene-3-carboxylic acid methyl ester (80, R⁴=Phenyl, R⁵=Methyl) (80 mg, 0.204 mmol) and 6-bromo-2-methylpyridin-3-amine (57 mg, 0.31 mmol) in toluene (2 mL) was treated with LiHMDS (1 M solution in THF, 0.61 mL, 0.61 mmol). The mixture was stirred for about 15 min and diluted with EtOAc (20 mL) then extracted with saturated aqueous NH₄Cl (˜10 mL) diluted with water (˜5 mL). The organic layer was dried over MgSO₄, filtered and concentrated under reduced pressure. The material was purified on silica gel (4 g) eluting with a gradient of 0-10% MeOH in DCM. Product containing fractions were collected and concentrated under reduced pressure. The material was sonicated with water (˜15 mL) then the solids were collected by filtration and dried at about 70° C. under vacuum to give 7aS,9R,11aS-11a-Benzyl-9-ethyl-9-hydroxy-6,7,7a,8,9,10,11,11a-octahydro-5H-dibenzo[a,c]cycloheptene-3-carboxylic acid (6-bromo-2-methyl-pyridin-3-yl)-amide (86, R⁴=Phenyl, R⁵=Methyl) (88 mg, 79%). LC/MS, method 2, Rₜ=2.86 min; MS m/z: 547 549 (M+H)⁺. The crude product was used in the next step as is.

### Step 2: (7aS,9R,11aS)-11a-Benzyl-9-ethyl-9-hydroxy-6,7,7a,8,9,10,11,11a-octahydro-5H-dibenzo[a,c]cycloheptene-3-carboxylic acid [2-methyl-6-(2H-pyrazol-3-yl)-pyridin-3-yl]-amide (87, R⁴=Phenyl, R⁵=Methyl, R⁷=(2H-pyrazol-3-yl)

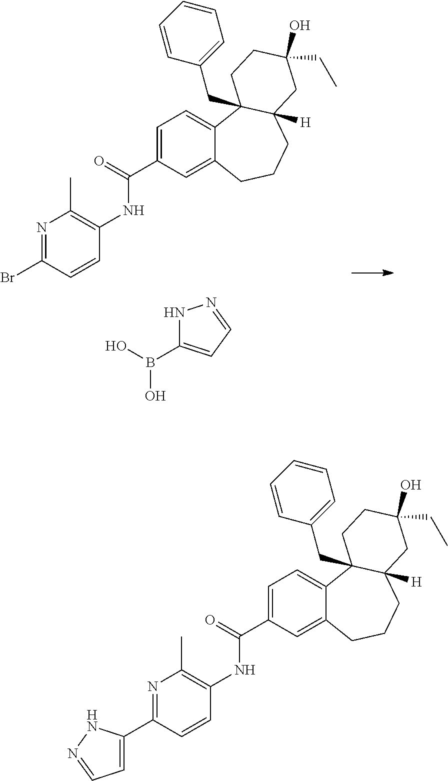

(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (6-bromo-2-methyl-pyridin-3-yl)-amide (86, R⁴=Phenyl, R⁵=Methyl) (50 mg, 0.091 mmol), 1H-pyrazol-5-ylboronic acid [Frontier] (20 mg, 0.18 mmol), Na₂CO₃ (39 mg, 0.37 mmol) and PdCl₂(PPh₃)₂ (13 mg, 0.018 mmol) in a mixture of DME (2 mL), EtOH (0.6 mL) and water (0.8 mL) was added to a microwave vial. The mixture was heated in a CEM microwave at about 150° C. for about 2.5 h (250 psi maximum pressure, 5 min ramp, 300 max watts). The mixture was partitioned between EtOAc (20 mL) and water (15 mL). The organic layer was washed with saturated aqueous NaCl (˜10 mL) then dried over MgSO₄, filtered and concentrated under reduced pressure. The material was purified on silica gel (4 g) eluting with a gradient of 0-7.5% MeOH in DCM. The fractions with the desired material were concentrated under reduced pressure to give a glass. The material was dissolved in MeOH (˜2 mL) then water (˜12 mL) was added. The mixture was concentrated under reduced pressure to remove MeOH. The mixture was allowed to stand at rt overnight then the solids were collected by filtration to give (7aS,9R,11aS)-11a-benzyl-9-ethyl-9-hydroxy-6,7,7a,8,9,10,11,11a-octahydro-5H-dibenzo[a,c]cycloheptene-3-carboxylic acid [2-methyl-6-(2H-pyrazol-3-yl)-pyridin-3-yl]-amide (87, R⁴=Phenyl, R⁵=Methyl, R⁷=2H-pyrazol-3-yl (25.1 mg, 51%); LC/MS, method 2, Rₜ=2.36 min; MS m/z: 535 (M+H)⁺; ¹H NMR (400 MHz, DMSO-d₆) δ 13.35 (s, 0.2H), 12.98 (s, 0.6H), 9.94 (bs, 1H), 7.83-7.77 (m, 4H), 7.57 (dd, J=8.2, 2.2 Hz, 1H), 7.08-7.04 (m, 3H), 6.84-6.80 (m, 2H), 6.61-6.58 (m, 2H), 3.89 (s, 1H), 3.58 (d, J=12.8 Hz, 1H), 3.30-3.27 (m, 1H), 3.01-3.08 (m, 1H), 2.61 (d, J=12.8 Hz, 1H), 2.48 (s, 3H), 2.46-2.40 (m, 1H), 1.94-1.86 (m, 1H), 1.85-1.73 (m, 2H), 1.50-1.66 (m, 2H), 1.41-1.44 (m, 1H), 1.29-1.37 (m, 1H), 1.05-1.28 (m, 5H), 0.71 (t, J=7.4 Hz, 3H)

Additional examples, prepared in a manner similar to the preparation of Example #86 are listed in Table 6.

TABLE 6

| | | | LC/MS | |
|---|---|---|---|---|
| Ex. | Boronic acid/ | | method/ | m/z ESI+ |
| # | boronate | Product | Rₜ min | (M + H)⁺ |
| 88 | 4-(4,4,5,5- | Compound 87 (7aS,9R,11aS) | 2/2.15 | 535 |
| | Tetramethyl- | (R⁴ = Phenyl, | | |
| | 1,3,2-dioxa- | R⁵ = Methyl, | | |
| | borolan-2-yl)- | R⁷ = 1H-Pyrazol-4-yl) | | |
| | 1H-pyrazole | | | |

### Example #89

### (7aS,9R,11aS)-11a-Benzyl-9-ethyl-9-hydroxy-6,7,7a,8,9,10,11,11a-octahydro-5H-dibenzo[a,c]cycloheptene-3-carboxylic acid methyl-(2-methyl-pyridin-3-yl)-amide

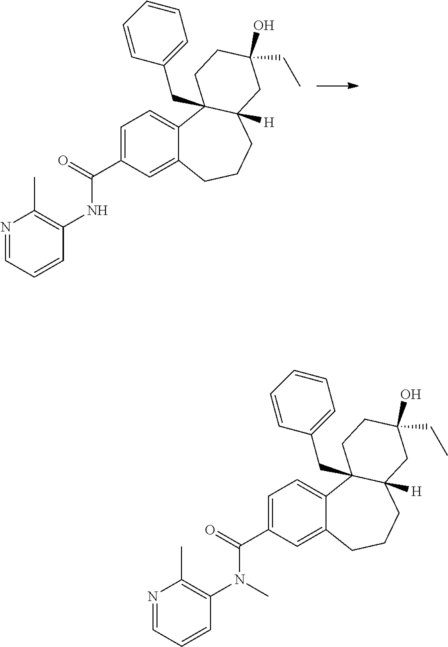

(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide (85, R⁴=Phenyl, R⁵=Methyl, R⁶=2-Methyl-pyridin-3-yl) (0.055 g, 0.12 mmol) (prepared using (7aS,9R,11aS)-11a-benzyl-9-ethyl-9-hydroxy-6,7,7a,8,9,10,11,11a-octahydro-5H-dibenzo[a,c]cycloheptene-3-carboxylic acid methyl ester and 2-methylpyridin-3-amine in a manner similar to the preparation of Example 68), was dissolved in DMF (3 mL) was and treated with NaH (60 wt % dispersion in mineral oil, 0.006 g, 0.14 mmol). After about 10 min, iodomethane (0.01 mL, 0.14 mmol) was added. After about 15 min, the mixture was treated with saturated aqueous NH₄Cl (˜4 mL) and water (20 mL). The mixture was extracted with EtOAc (15 mL then 10 mL). The combined organics were washed with water (25 mL) then saturated aqueous NaCl (15 mL), dried over MgSO₄, filtered and concentrated under reduced pressure. The material was purified on silica gel (4 g) eluting with a gradient of 0-10% MeOH in DCM. The product containing fractions were combined and concentrated under reduced pressure. The material was dissolved in MeOH (˜1 mL) then water (˜20 mL) was added. The mixture was concentrated under reduced pressure to remove MeOH then the solids were collected by filtration and washed with water (˜4 mL). The material was dried under vacuum at about 70° C. to give (7aS,9R,11aS)-11a-benzyl-9-ethyl-9-hydroxy-6,7,7a,8,9,10,11,11a-octahydro-5H-dibenzo[a,c]cycloheptene-3-carboxylic acid methyl-(2-methyl-pyridin-3-yl)-amide (0.036 g, 64%); LC/MS, method 2, Rₜ=2.41 min; MS m/z: 483 (M+H)⁺; ¹H NMR (95° C.) (400 MHz, DMSO-d₆) δ 8.32-8.34 (m, 1H), 7.79-7.64 (m, 1H), 7.39-7.13 (m, 1H), 7.06-6.95 (m, 4H), 6.80-6.66 (m, 1H), 6.58-6.29 (m, 2H), 6.21-6.18 (m, 1H), 3.82 (s, 1H), 3.24-2.93 (m, 2H), 2.98-2.95 (m, 4H), 2.76-2.66 (m, 1H), 2.36-2.26 (m, 2H), 2.30 (s, 3H), 0.93-1.69 (m, 11H), 0.65 (t, J=7.5 Hz, 3H)

### Example #90 and #91

### Chiral separation of (7aR,9R,11aS)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide; compound with (7aS,9R,11aR)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide (12, R²=Benzyl, R³=Methyl)

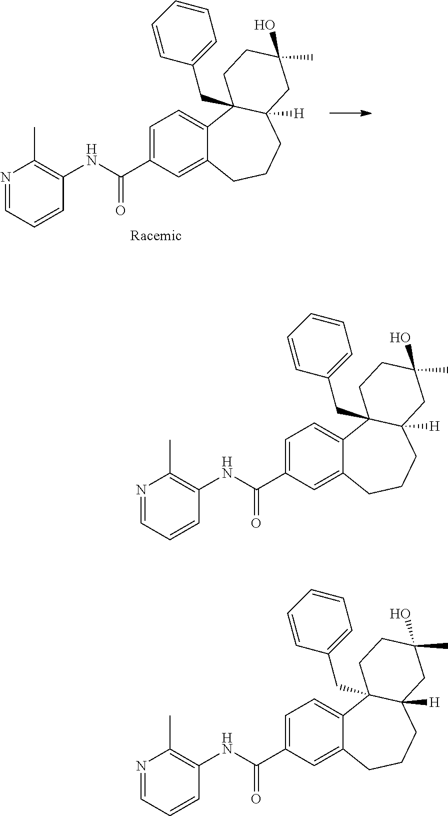

Example #9 (12, R²=Benzyl, R³=Methyl) (0.305 g) was purified using a chiral chromatography isocratic separation method. The method used 10% EtOH in heptane contining 0.1% DEA with a Daicel IB column (20×250 mm) to give first example 90, (7aR,9R,11aS)-11a-benzyl-9-hydroxy-9-methyl-6,7,7a,8,9,10,11,11a-octahydro-5H-dibenzo[a,c]cycloheptene-3-carboxylic acid (2-methylpyridin-3-yl)-amide (12, R²=Benzyl, R³=Methyl) (0.128 g) and second example 91, (7aS,9R,11aR)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide (20, R²=Benzyl, R³=Methyl) (0.120 g) NMR and LC/MS data for single isomers was essentially identical to the racemic mixture.

### Example #92

### (7aS,9R,11aR)-11a-Ethyl-9-hydroxy-9-(2,2,2-trifluoro-ethoxymethyl)-6,7,7a,8,9,10,11,11a-octahydro-5H-dibenzo cycloheptene-3-carboxylic acid (2-methyl-pyridin-3-yl)-amide: compound with (7aR,9S,11aS)-11a-ethyl-9-hydroxy-9-(2,2,2-trifluoro-ethoxymethyl)-6,7,7a,8,9,10,11,11a-octahydro-5H-dibenzo[a,c]cycloheptene-3-carboxylic acid (2-methyl-pyridin-3-yl)-amide (77, R⁴=Methyl, R⁵=2,2,2-Trifluoroethoxy)

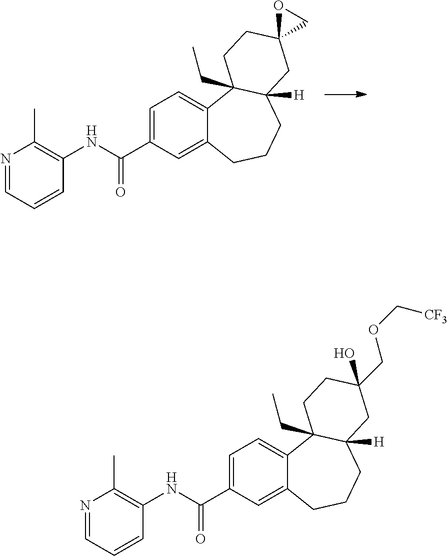

In a round flask, (+/−) Compound 76 (R⁴=Methyl) (0.065 g, 0.17 mmol) was dissolved in 2,2,2-trifluoroethanol (1.0 mL, 14 mmol), followed by the addition of Na₂CO₃ (0.023 g, 0.22 mmol). The mixture was heated to about 60° C. for about 18 h, then heated to about 75° C. for about 18 h. The mixture was cooled and concentrated in vacuo, diluted with water (5 mL) and extracted with EtOAc (10 mL). The organic layer was dried over MgSO₄ and concentrated under reduced pressure. The residue was purified on silica gel (12 g) eluting with a gradient of 0-5% MeOH in EtOAc. Fractions containing product were combined and concentrated under reduced pressure. The residue was dissolved in a minimum of MeOH then diluted with water. The resulting precipitate was collected by filtration and dried under reduced pressure to give, (7aS,9R,11aR)-11a-ethyl-9-hydroxy-9-(2,2,2-trifluoro-ethoxymethyl)-6,7,7a,8,9,10,11,11a-octahydro-5H-dibenzo[a,c]cycloheptene-3-carboxylic acid (2-methyl-pyridin-3-yl)-amide: compound with (7aR,9S,11aS)-11a-ethyl-9-hydroxy-9-(2,2,2-trifluoro-ethoxymethyl)-6,7,7a,8,9,10,11,11a-octahydro-5H-dibenzo[a,c]cycloheptene-3-carboxylic acid (2-methyl-pyridin-3-yl)-amide (77, R⁴=Methyl, R⁵=2,2,2-Trifluoroethoxy) (0.010 g, 12%) as a white solid. LC/MS, method 2, Rₜ=2.21 min, MS m/z 491 (M+H)^{+ 1}H NMR (400 MHz, DMSO-d₆) δ 9.94 (s, 1H), 8.33 (dd, J=4.7, 1.5 Hz, 1H), 7.79-7.69 (m, 3H), 7.38 (d, J=8.4 Hz, 1H), 7.27 (dd, J=8.0, 4.8 Hz, 1H), 4.36 (s, 1H), 3.98 (q, J=9.4 Hz, 2H), 3.22 (s, 2H), 3.05-2.93 (m, 1H), 2.94-2.83 (m, 1H), 2.43 (s, 3H), 2.36-2.19 (m, 3H), 2.13-2.00 (m, 1H), 1.63-152 (m, 7H), 1.25-1.06 (m, 2H), 0.61 (t, J=7.3 Hz, 3H).

Additional examples, prepared in a manner similar to the preparation of Example #18 or Example #92 are listed in Table 2.

TABLE 2

| | | | | | | Chiral |
|---|---|---|---|---|---|---|
| | | | | LC/MS | m/z | method/ |
| | | | | method/ | ESI+ | Order of |
| Ex.# | Epoxide | Alcohol | Product | Rₜ min | (M + H)⁺ | elution |
| 93 | Compound | Ethanol | Compound 29 | 2/2.32 | 499 | 8/First |
| | 28 (R² = | | (7aS,9R,11aS) | | | |
| | Benzyl) | | (R = Ethyl, R² = | | | |
| | | | Benzyl) | | | |
| 94 | Compound | Ethanol | Compound 29 | 2/2.32 | 499 | 8/Second |
| | 28 (R² = | | (7aR,9S,11aR) | | | |
| | Benzyl) | | (R = Ethyl, R² = | | | |
| | | | Benzyl) | | | |
| 95 | Compound | 2,2,2- | Compound 29 | 2/2.47 | 553 | NA |
| | 28 (R² = | Trifluoroethanol | (7aS,9R,11aS) | | | |
| | Benzyl) | | compound with | | | |
| | | | (7aR,9S,11aR) | | | |
| | | | (R = Trifluoroethyl, R² = | | | |
| | | | Benzyl) | | | |
| 96 | Compound | Oxetan-3- | Compound 29 | 2/2.03 | 527 | NA |
| | 28 (R² = | ol | (7aS,9R,11aS) | | | |
| | Benzyl) | | compound with | | | |
| | | | (7aR,9S,11aR) | | | |
| | | | (R = Oxetan-3-yl, R² = | | | |
| | | | Benzyl) | | | |
| 97 | Compound | 2-Propanol | Compound 29 | 2/2.45 | 513 | NA |
| | 28 (R² = | | (7aS,9R,11aS) | | | |
| | Benzyl) | | compound with | | | |
| | | | (7aR,9S,11aR) | | | |
| | | | (R = Isopropyl, R² = | | | |
| | | | Benzyl) | | | |
| 98 | Compound | 1-Propanol | Compound 29 | 2/2.47 | 513 | 6/First |
| | 28 (R² = | | (7aS,9R,11aS) | | | |
| | Benzyl) | | (R = Propyl, R² = | | | |
| | | | Benzyl) | | | |
| 99 | Compound | 1-Propanol | Compound 29 | 2/2.47 | 513 | 6/Second |
| | 28 (R² = | | (7aR,9S,11aR) | | | |
| | Benzyl) | | (R = Propyl, R² = | | | |
| | | | Benzyl) | | | |
| 100 | Compound | 1,1,1- | Compound 29 | 2/2.45 | 567 | NA |
| | 28 (R² = | Trifluoro- | (7aS,9R,11aS) | | | |
| | Benzyl) | propan-2- | compound with | | | |
| | | ol | (7aR,9S,11aR) | | | |
| | | | (R = 1,1,1-Trifluoro- | | | |
| | | | propan-2-yl, R² = | | | |
| | | | Benzyl) | | | |
| 101 | Compound | 1-Propanol | Compound 29 | 2/2.44 | 513 | NA |
| | 28 (R² = | | (7aS,9R,11aS) | | | |
| | Benzyl) | | compound with | | | |
| | | | (7aR,9S,11aR) | | | |
| | | | (R = 1-Propyl, R² = | | | |
| | | | Benzyl) | | | |
| 102 | Compound | Tetrahydro- | Compound 29 | 2/2.21 | 555 | NA |
| | 28 (R² = | pyran-4-ol | (7aS,9R,11aS) | | | |
| | Benzyl) | | compound with | | | |
| | | | (7aR,9S,11aR) | | | |
| | | | (R = Tetrahydro-pyran- | | | |
| | | | yl, R² = Benzyl) | | | |
| 103 | Compound | Phenol | Compound 29 | 2/2.52 | 547 | NA |
| | 28 (R² = | | (7aS,9R,11aS) | | | |
| | Benzyl) | | compound with | | | |
| | | | (7aR,9S,11aR) | | | |
| | | | (R = Phenyl, R² = | | | |
| | | | Benzyl) | | | |
| 104 | Compound | 2- | Compound 29 | 2/1.86 | 471 | NA |
| | 28 (R² = | Methanesulfonyl | (7aS,9R,11aS) | | | |
| | Benzyl) | ethanol | compound with | | | |
| | | | (7aR,9S,11aR) | | | |
| | | | (R = H, R² = Benzyl) | | | |
| 105 | Compound | 2- | Compound 29 | 2/1.99 | 577 | NA |
| | 28 (R² = | Methanesulfonyl | (7aS,9R,11aS) | | | |
| | Benzyl) | ethanol | compound with | | | |
| | | | (7aR,9S,11aR) | | | |
| | | | (R = 2- | | | |
| | | | Methanesulfonyl ethanol- | | | |
| | | | yl, R² = Benzyl) | | | |
| 105A | Compound | ethanol | Compound 77 | 1/0.67 | 437 | NA |
| | 76 (R⁴ = | | (7aS,9R,11aS) | | | |
| | Methyl, R⁵ = | | compound with | | | |
| | Ethoxy) | | (7aR,9S,11aR) | | | |
| | | | (R⁴ = Methyl, R⁵ = | | | |
| | | | Ethoxy) | | | |

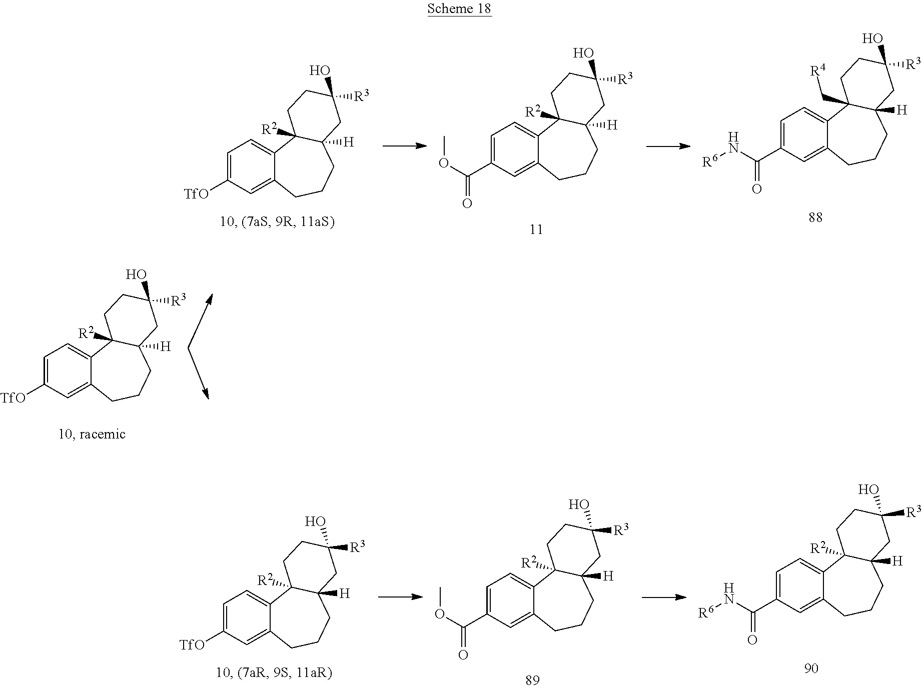

### Example #106

### (7aR,9R,11aS)-11a-Benzyl-9-hydroxy-9-trifluoromethyl-6,7,7a,8,9,10,11,11a-octahydro-5H-dibenzo[a,c]cycloheptene-3-carboxylic acid (2-methyl-pyridin-3-yl)-amide (88, R²=Benzyl, R³=Trifluoromethyl) A-1337940 and Example #107: (7aS,9S,11aR)-11a-Benzyl-9-hydroxy-9-trifluoromethyl-6,7,7a,8,9,10,11,11a-octahydro-5H-dibenzo[a,c]cycloheptene-3-carboxylic acid (2-methyl-pyridin-3-yl)-amide (90, R²=Benzyl, R³=Trifluoromethyl)

### Preparation of trifluoromethanesulfonic acid (7aR,9R,11aS)-11a-benzyl-9-hydroxy-9-trifluoromethyl-6,7,7a,8,9,10,11,11a-octahydro-5H-dibenzo[a,c]cyclohepten-3-yl ester (10, 7aR,9R,11aS, R²=Benzyl, R³=Trifluoromethyl) and trifluoro-methanesulfonic acid (7aS,9S,11aR)-11a-benzyl-9-hydroxy-9-trifluoromethyl-6,7,7a,8,9,10,11,11a-octahydro-5H-dibenzo[a,c]cyclohepten-3-yl ester (10, 7aS,9S,11aR, R²=Benzyl, R³=Trifluoromethyl)

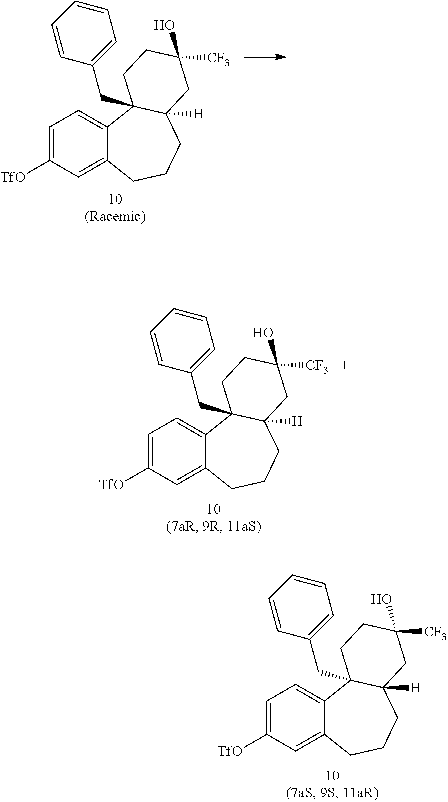

Trifluoro-methanesulfonic acid (7aR,9R,11aS)-11a-benzyl-9-hydroxy-9-trifluoromethyl-6,7,7a,8,9,10,11,11a-octahydro-5H-dibenzo[a,c]cyclohepten-3-yl ester; compound with trifluoro-methanesulfonic acid (7aS,9S,11aR)-11a-benzyl-9-hydroxy-9-trifluoromethyl-6,7,7a,8,9,10,11,11a-octahydro-5H-dibenzo[a,c]cyclohepten-3-yl ester (10, R²=Benzyl, R³=Trifluoromethyl) was purified by chiral chromatography isocratic separation method using 1% EtOH in heptane with 0.1% DEA with Daicel IB column (20×250 mm) to give trifluoro-methanesulfonic acid (7aR,9R,11aS)-11a-benzyl-9-hydroxy-9-trifluoromethyl-6,7,7a,8,9,10,11,11a-octahydro-5H-dibenzo[a,c]cyclohepten-3-yl ester [Rₜ=23.30 min] and trifluoro-methanesulfonic acid (7aS,9S,11aR)-11a-benzyl-9-hydroxy-9-trifluoromethyl-6,7,7a,8,9,10,11,11a-octahydro-5H-dibenzo[a,c]cyclohepten-3-yl ester [Rₜ=32.68 min]. Single enantiomers were modified to final products (7aR,9R,11aS)-11a-benzyl-9-hydroxy-9-trifluoromethyl-6,7,7a,8,9,10,11,11a-octahydro-5H-dibenzo[a,c]cycloheptene-3-carboxylic acid (2-methyl-pyridin-3-yl)-amide (88, R²=Benzyl, R³=Trifluoromethyl) and (7aS,9S,11aR)-11a-benzyl-9-hydroxy-9-trifluoromethyl-6,7,7a,8,9,10,11,11a-octahydro-5H-dibenzo[a,c]cycloheptene-3-carboxylic acid (2-methyl-pyridin-3-yl)-amide (90, R²=Benzyl, R³=Trifluoromethyl) according to the route outlined in Scheme 18, in a manner similar to the preparation of Example 3. NMR and LC/MS data for single isomers was essentially identical to the racemic mixture.

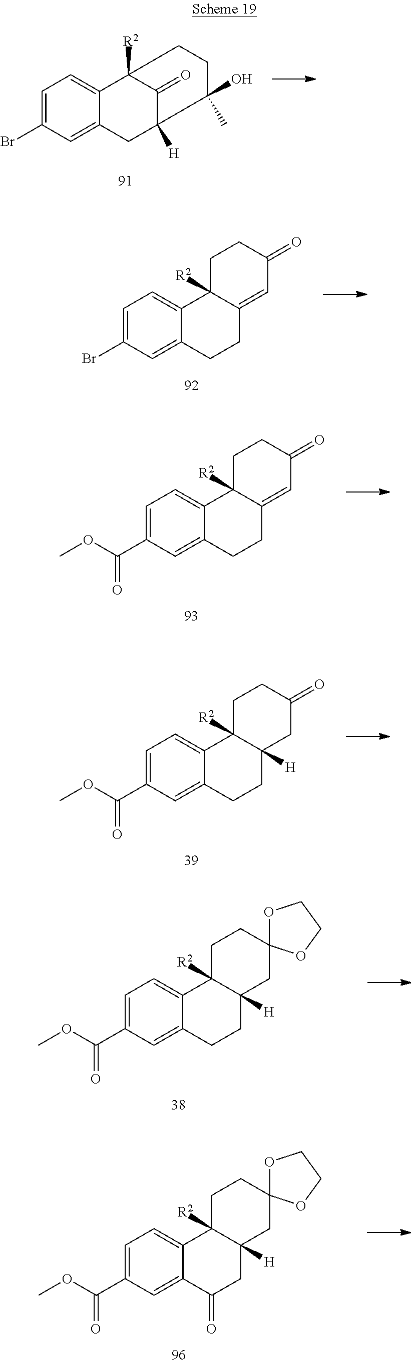

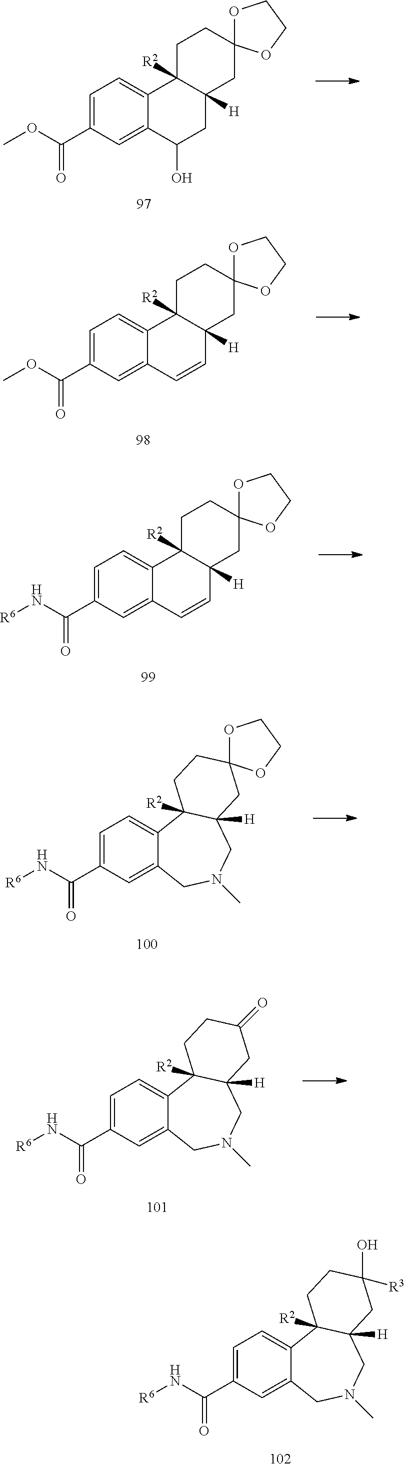

### Example #108 and #109

### (4aS,11bS)-11b-Benzyl-6-methyl-N-(2-methylpyridin-3-yl)-3-oxo-2,3,4,4a,5,6,7,11b-octahydro-1H-dibenzo[c,e]azepine-9-carboxamide (101, R²=Benzyl, R⁶=2-Methyl-pyridin-3-yl) and (3S,4aS,11bS)-11b-benzyl-3-hydroxy-6-methyl-N-(2-methylpyridin-3-yl)-2,3,4,4a,5,6,7,11b-octahydro-1H-dibenzo[c,e]azepine-9-carboxamide (102, R²=Benzyl, R³=H, R⁶=2-Methyl-pyridin-3-yl)

### Step #1: (S)-4a-Benzyl-7-bromo-4,4a,9,10-tetrahydrophenanthren-2(3H)-one (92, R²=Benzyl)

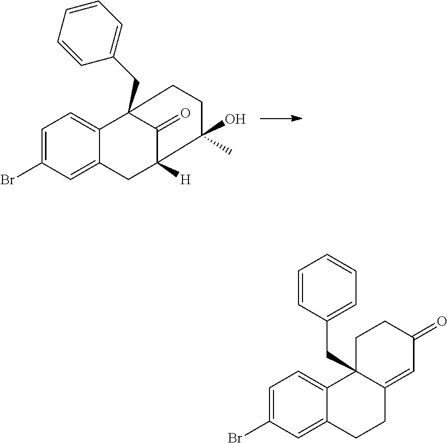

Sodium ethoxide (21 wt % in EtOH, 84.0 g, 260 mmol) was added to a solution of (1R,10R)-1-benzyl-5-bromo-10-hydroxy-10-methyltricyclo[7.3.1.0 2,7]trideca-2,4,6-trien-13-one (1.0 kg, 2.60 mol) (91, R²=Benzyl) (prepared as described in WO 2008093236 A1) and EtOH (10 L). The reaction mixture was warmed to about 80° C. After about 30 min, the reaction mixture was allowed to cool to rt. The solvent was distilled off. The residue was dissolved in MTBE (20 L) and then washed with saturated aqueous NaCl (15 L). The aqueous layer was extracted with MTBE (5 L). The combined organics were dried over MgSO₄, filtered, and concentrated under reduced pressure. The residue was purified on silica gel (10 kg) eluting with heptane then 10% EtOAc in heptane. The product containing fractions were combined and concentrated under reduced pressure to afford (S)-4a-benzyl-7-bromo-4,4a,9,10-tetrahydrophenanthren-2(3H)-one (92, R²=Benzyl) (929 g, 97%). HPLC, Zorbax RX-8 column, 95% 0.1% H₃PO₄, (buffer), 5% MeCN to 15 min, 5 min hold time, flow 1.5 mL/min, column temperature 35° C., 14.98 min, LC/MS, method 3, Rₜ=2.89 min, MS m/z 367/369 (M+H)⁺, ¹H NMR (400 MHz, DMSO-d₆) δ 7.40 (dd, J=8.5, 2.1 Hz, 1H), 7.34 (d, J=8.6 Hz, 1H), 7.30 (d, J=2.1 Hz, 1H), 7.18-7.11 (m, 3H), 6.81-6.75 (m, 2H), 5.92 (s, 1H), 3.29 (d, J=13.2 Hz, 1H), 3.24 (d, J=13.2 Hz, 1H), 2.88-2.79 (m, 1H), 2.76-2.60 (m, 2H), 2.56-2.47 (m, 1H), 2.38-2.24 (m, 2H), 1.95-1.80 (m, 2H).

### Step #2: (S)-Methyl 4b-benzyl-7-oxo-4b,5,6,7,9,10-hexahydrophenanthrene-2-carboxylate (93, R²=Benzyl)

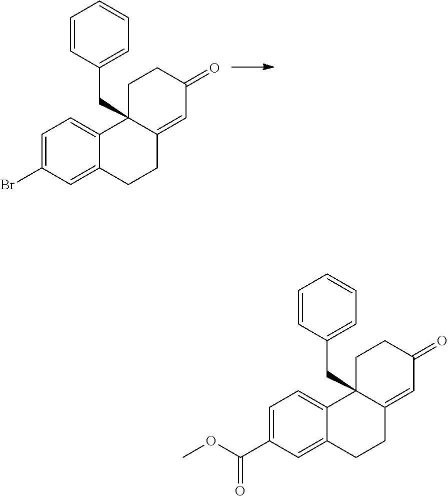

1,1′-Bis(diphenylphosphino)ferrocenedichloropalladium(II) dichloromethane adduct (0.563 g, 0.689 mmol), (S)-4a-benzyl-7-bromo-4,4a,9,10-tetrahydrophenanthren-2(3H)-one (92, R²=Benzyl) (55.0 g, 138 mmol), TEA (38.4 mL, 276 mmol) and MeOH (500 mL) were added under nitrogen to a 2 L Parr stirred reactor. The reactor was purged with nitrogen and then carbon monoxide. The mixture was agitated for about 15 h at about 100° C. under about 60 psi of carbon monoxide. DCM (300 mL) was added. The reaction mixture was filtered through a Buchner funnel containing a GF/F glass fiber filter to remove the catalyst rinsing with DCM. The organics were washed with 1 N aqueous HCl (500 mL), an aqueous solution of 7% cysteine and 5% KHCO₃ (2×650 mL), dried over Na₂SO₄, and filtered. The solution was concentrated to about 150 g under reduced pressure and then filtered through a plug of silica (200 g) rinsing with DCM (2 L). The organics were concentrated to about 300 g under reduced pressure. MeOH (500 mL) was added and then the solution was concentrated to about 300 g under reduced pressure. MeOH (500 mL) was added and then the solution was concentrated to about 300 g under reduced pressure. The oil was cooled in a bath of ice/water. The mixture was filtered rinsing with cold MeOH to afford, after drying under reduced pressure in a vacuum oven, (S)-methyl 4b-benzyl-7-oxo-4b,5,6,7,9,10-hexahydrophenanthrene-2-carboxylate (93, R²=Benzyl) (28.4 g, 59%) as a white solid. LC/MS, method 3, Rₜ=2.38 min, MS m/z 347 (M+H)⁺. ¹H NMR (400 MHz, D DMSO-d₆) δ 7.79 (dd, J=8.3, 1.8 Hz, 1H), 7.67 (d, J=1.7 Hz, 1H), 7.55 (d, J=8.4 Hz, 1H), 7.17-7.08 (m, 3H), 6.78-6.70 (m, 2H), 5.93 (s, 1H), 3.84 (s, 3H), 3.34 (d, J=13.2 Hz, 1H), 3.28 (d, J=13.2 Hz, 1H), 2.96-2.85 (m, 1H), 2.80-2.64 (m, 2H), 2.62-2.53 (m, 1H), 2.41-2.26 (m, 2H), 197-1.80 (m, 2H).

### Step #3: (4bS,8aS)-Methyl 4b-benzyl-7-oxo-4b,5,6,7,8,8a,9,10-octahydrophenanthrene-2-carboxylate (39, R²=Benzyl)

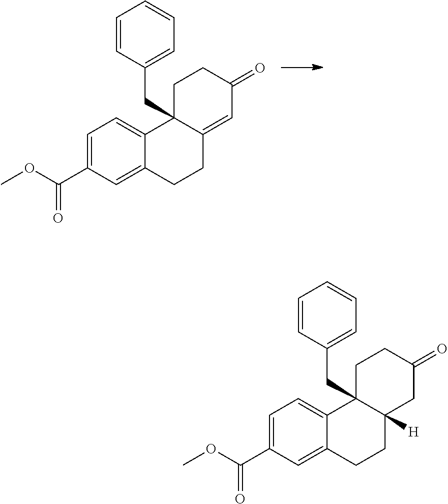

(S)-Methyl 4b-benzyl-7-oxo-4b,5,6,7,9,10-hexahydrophenanthrene-2-carboxylate (93, R²=Benzyl) (58.0 g, 167 mmol), 5% Pd/C (6.1 g), THF (320 mL) and pyridine (80 mL) were added under nitrogen to a 1.8 L SS pressure bottle. The reactor was purged with nitrogen and then hydrogen. The mixture was agitated for about 2 h at rt under about 40 psi of hydrogen. The reaction mixture was filtered through a Buchner funnel containing a GF/F glass fiber filter to remove the catalyst rinsing with THF. The combined filtrates were concentrated under reduced pressure. The oil was dissolved in EtOAc (300 mL) and the resulting solution was washed with 0.2 M aqueous CuSO₄ (2×100 mL and 2×200 mL). The organic layer was dried over Na₂SO₄, filtered, and concentrated under reduced pressure. The residue was purified on silica gel (750 g) using a gradient of 10-30% EtOAc in heptane. The product containing fractions were combined and concentrated under reduced pressure to afford a 96:4 mixture of diastereomers favoring (4bS,8aS)-methyl 4b-benzyl-7-oxo-4b,5,6,7,8,8a,9,10-octahydrophenanthrene-2-carboxylate (39, R²=Benzyl) (57.0 g, 98%) as a thick oil. LC/MS, method 3, Rₜ=2.49 min, MS m/z 366 (M+H₂O)⁺. ¹H NMR (400 MHz, DMSO-d₆) δ 7.73 (d, J=7.9 Hz, 1H), 7.72 (s, 1H), 7.46 (d, J=8.0 Hz, 1H), 7.22-7.15 (m, 3H), 6.95-6.89 (m, 2H), 3.84 (s, 3H), 3.11 (s, 2H), 2.93-2.70 (m, 2H), 2.47-2.26 (m, 3H), 2.21-1.88 (m, 5H), 1.61-1.49 (m, 1H).

### Step #4: (4a′S,10a′S)-Methyl 4a′-benzyl-3′,4′,4a′,9′,10′,10a′-hexahydro-1′H-spiro[[1,3]dioxolane-2,2′-phenanthrene]-7′-earboxylate (38, R²=Benzyl)

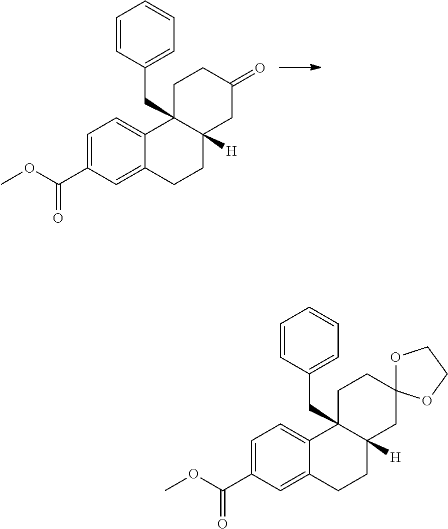

Ethylene glycol (1.30 mL, 23.3 mmol), trimethyl orthoformate (4.00 mL, 36.5 mmol), and toluene-4-sulfonic acid hydrate (0.440 g, 2.31 mmol) were respectively added, each in one portion, to a solution of (4bS,8aS)-methyl 4b-benzyl-7-oxo-4b,5,6,7,8,8a,9,10-octahydrophenanthrene-2-carboxylate (39, R²=Benzyl) (4.00 g, 11.5 mmol) and DCM (60 mL) under a nitrogen atmosphere. After about 4 h, the pale green solution was poured into a solution of saturated aqueous NaHCO₃ (75 mL) and water (25 mL). The layers were separated and the aqueous layer was extracted with DCM (50 mL). The combined organics were dried over Na₂SO₄, filtered, and concentrated under reduced pressure. The residue was purified on silica gel (120 g) using a gradient of 0-40% EtOAc in heptane. The product containing fractions were combined and concentrated under reduced pressure to afford (4a′S,10a′S)-methyl 4a′-benzyl-3′,4′,4a′,9′,10′,10a′-hexahydro-1′H-spiro[[1,3]dioxolane-2,2′-phenanthrene]-7′-carboxylate (38, R²=Benzyl) (3.55 g, 79%) as a white foam. LC/MS, method 1, Rₜ=0.95 min, MS m/z 393 (M+H)⁺. ¹H NMR (400 MHz, DMSO-d₆) δ 7.75-7.71 (m, 1H), 7.67-7.62 (m, 1H), 7.25-7.13 (m, 4H), 6.96-6.91 (m, 2H), 3.83 (s, 3H), 3.81-3.70 (m, 4H), 2.93-2.69 (m, 4H), 2.45-2.37 (m, 1H), 2.23-2.14 (m, 1H), 1.99-1.89 (m, 1H), 1.71-1.45 (m, 4H), 1.34-1.20 (m, 1H), 1.13-1.02 (m, 1H).

### Step #5: (4a′S,10a′S)-Methyl 4a′-benzyl-9′-oxo-3′,4′,4a′,9′,10′,10a′-hexahydro-1′H-spiro[[1,3]dioxolane-2,2′-phenanthrene]-7′-carboxylate (96, R²=Benzyl)

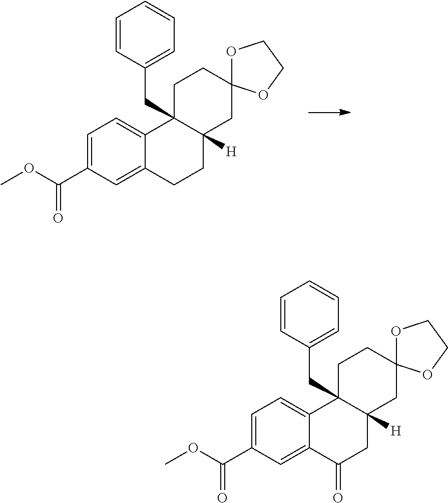

A solution of (4a′S,10a′S)-methyl 4a′-benzyl-3′,4′,4a′,9′,10′,10a′-hexahydro-1′H-spiro[[1,3]dioxolane-2,2′-phenanthrene]-7′-carboxylate (38, R²=Benzyl) (3.64 g, 9.04 mmol) and DCM (80 mL) was added to a 250 mL Erlenmeyer flask with a large stir bar. Copper(II) sulfate pentahydrate (9.00 g, 36.0 mmol), potassium permanganate (5.70 g, 36.1 mmol), water (10 mL), and pyridine (2.90 mL, 35.9 mmol) were added respectively, each in one portion. The mixture was left to vigorously stir under air for about 43 h. Na₂SO₄ (40 g) was added. After about 2 h, the mixture was filtered through Celite® rinsing with DCM (6×50 mL). The volatiles were removed under reduced pressure. The residue was slurried between water (100 mL) and EtOAc (200 mL) and then filtered rinsing with EtOAc. The layers were separated and the organics washed with water (2×100 mL). The organics were dried over Na₂SO₄, filtered, and concentrated under reduced pressure. The residue was purified on silica gel (120 g) using a gradient of 0-15% EtOAc in DCM. The product containing fractions were combined and concentrated under reduced pressure to afford a light yellow foam. The residue was dissolved in DCM (200 mL) and then washed with 0.1 M aqueous EDTA tetrasodium salt (100 mL) and water (100 mL), dried over Na₂SO₄, filtered, and concentrated under reduced pressure to afford (4a′S,10a′S)-methyl 4a′benzyl-9′-oxo-3′,4′,4a′,9′,10′,10a′-hexahydro-1′H-spiro[[1,3]dioxolane-2,2′-phenanthrene]-7′-carboxylate (96, R²=Benzyl) (2.19 g, 60%) as a light yellow foam. LC/MS, method 1, Rₜ=0.81 min, MS m/z 407 (M+H)⁺. ¹H NMR (400 MHz, DMSO) δ 8.52 (d, J=2.0 Hz, 1H), 8.08 (dd, J=8.2, 2.0 Hz, 1H), 7.28 (d, J=8.3 Hz, 1H), 7.26-7.20 (m, 3H), 6.98-6.92 (m, 2H), 3.89 (s, 3H), 3.83-3.70 (m, 4H), 3.47 (dd, J=18.0, 5.2 Hz, 1H), 3.03 (d, J=13.3 Hz, 1H), 2.92 (d, J=13.3 Hz, 1H), 2.44-2.24 (m, 3H), 1.78-1.68 (m, 1H), 1.68-1.60 (m, 1H), 1.58-1.49 (m, 1H), 1.17-1.02 (m, 2H).

### Step #6: (4a′S,10a′S)-Methyl 4a′-benzyl-9′-hydroxy-3′,4′,4a′,9′,10′,10a′-hexahydro-1′H-spiro[[1,3]dioxolane-2,2′-phenanthrene]-7′-carboxylate (97, R²=Benzyl)

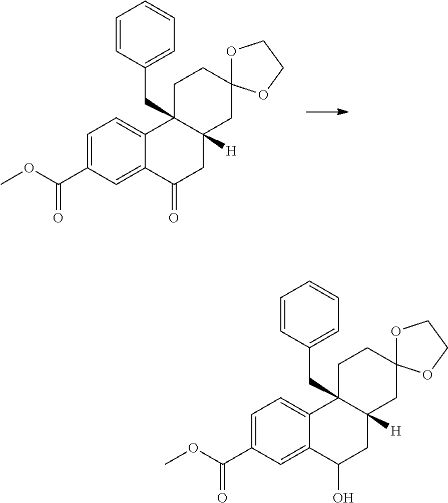

NaBH₄ (0.107 g, 2.83 mmol) was added portionwise over about 5 min to a solution of (4a′S,10a′S)-methyl 4a′-benzyl-9′-oxo-3′,4′,4a′,9′,10′,10a′-hexahydro-1′H-spiro[[1,3]dioxolane-2,2′-phenanthrene]-7′-carboxylate (96, R²=Benzyl) (1.32 g, 2.83 mmol) and MeOH (28 mL) under air cooled in an rt water bath. After about 30 min, the solution was concentrated to about 5 mL and then water (50 mL) and DCM (50 mL) were added. After vigorously stirring for about 30 min, the layers were separated and the aqueous phase was extracted with DCM (2×50 mL). The combined organics were washed with saturated aqueous NaCl (50 mL), dried over Na₂SO₄, filtered, and concentrated under reduced pressure. The residue was purified on silica gel (80 g) using a gradient of 0-40% EtOAc in DCM. The product containing fractions were combined and concentrated under reduced pressure to afford (4a′S,10a′S)-methyl 4a′-benzyl-9′-hydroxy-3′,4′,4a′,9′,10′,10a′-hexahydro-1′H-spiro[[1,3]dioxolane-2,2′-phenanthrene]-7′-carboxylate (97, R²=Benzyl) (1.06 g, 92%) as approximately a 2:1 mixture of alcohol diastereomers as a sticky ivory foam. LC/MS, method 1, Rₜ=0.73 min, MS m/z 391 (M−OH)⁺. ¹H NMR (400 MHz, DMSO-d₆) δ 8.19 (d, J=2.0 Hz, 0.33H), 8.12 (d, J=2.0 Hz, 0.67H), 7.78-7.73 (dd, J=8.2, 2.0 Hz, 0.67H), 7.71 (d, J=8.0, 2.0 Hz, 0.33H), 7.38-7.30 (m, 0.67H), 7.28-7.08 (m, 3.33H), 7.01-9.95 (m, 0.67H), 6.83-6.77 (m, 1.33H), 5.44-5.38 (m, 1H), 4.74-4.63 (m, 0.33H), 4.49-4.39 (m, 0.67H), 3.85 (s, 3H), 3.84-3.69 (m, 4H), 3.01-0.99 (m, 11H).

### Step #7: (4a′S,10a′R)-Methyl 4a′-benzyl-3′,4′,4a′,10a′-tetrahydro-1′H-spiro[[1,3]dioxolane-2,2′-phenanthrene]-7′-carboxylate (98, R²=Benzyl)

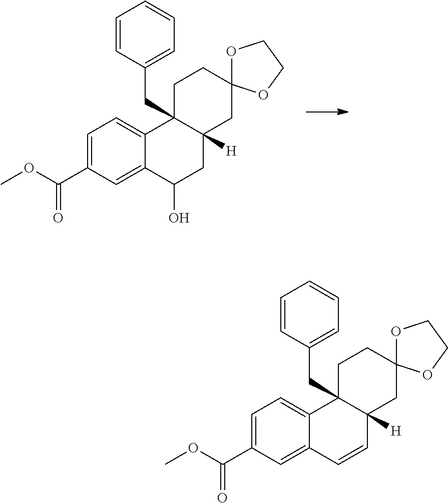

4 Å Molecular sieves (2.0 g) were added to a solution of (4a′S,10a′S)-methyl 4a′-benzyl-9′-hydroxy-3′,4′,4a′,9′,10′,10a′-hexahydro-1′H-spiro[[1,3]dioxolane-2,2′-phenanthrene]-7′-carboxylate (97, R²=Benzyl) (1.25 g, 2.60 mmol) and toluene (50 mL). After about 10 min, toluene-4-sulfonic acid hydrate (0.030 g, 0.16 mmol) was added. After about 5 min, the reaction mixture was warmed to about 60° C. After about 30 min, toluene-4-sulfonic acid hydrate (0.030 g, 0.16 mmol) was added. After about 2 h, the mixture was allowed to cool to rt and then filtered with an EtOAc rinse into saturated aqueous NaHCO₃ (50 mL) and EtOAc (50 mL). The layers were separated and the organics were washed with saturated aqueous NaCl (50 mL), dried over Na₂SO₄, filtered, and concentrated under reduced pressure. The residue was purified on silica gel (80 g) using a gradient of 0-20% EtOAc in heptane. The product containing fractions were combined and concentrated under reduced pressure to afford (4a′S,10aR)-methyl 4a′-benzyl-3′,4′,4a′,10a′-tetrahydro-1′H-spiro[[1,3]dioxolane-2,2′-phenanthrene]-7′-carboxylate (98, R²=Benzyl) (0.655 g, 64%) as a sticky ivory foam/colorless film. LC/MS, method 1, Rₜ=0.91 min, MS m/z 391 (M+H)⁺. ¹H NMR (400 MHz, DMSO-d₆) δ 7.80 (d, J=1.8 Hz, 1H), 7.72 (dd, J=8.0, 1.9 Hz, 1H), 7.71-7.13 (m, 3H), 7.06 (d, J=8.1 Hz, 1H), 6.78-6.72 (m, 2H), 6.70 (d, J=9.6 Hz, 1H), 6.19 (dd, J=9.5, 6.3 Hz, 1H), 3.85 (s, 3H), 3.83-3.71 (m, 4H), 2.76 (d, J=12.8 Hz, 1H), 2.57 (d, J=12.8 Hz, 1H), 2.38-2.30 (m, 1H), 2.27-2.17 (m, 1H), 1.78-1.67 (m, 1H), 1.65-1.51 (m, 2H), 1.41-1.30 (m, 1H), 0.95-0.83 (m, 1H).

### Step #8: (4a′S,10a′R)-4a′-Benzyl-N-(2-methylpyridin-3-yl)-3′,4′,4a′,10a′-tetrahydro-1′H-spiro[[1,3]dioxolane-2,2′-phenanthrene]-7′-carboxamide (99, R²=Benzyl, R⁶=2-Methylpyridin-3-yl)

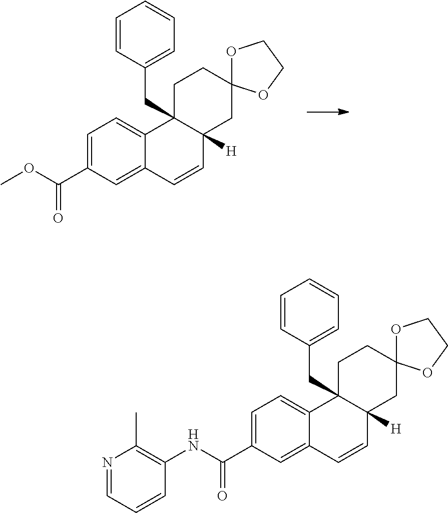

LiHMDS (1 M solution in THF, 3.50 mL, 3.50 mmol) was added dropwise over about 5 min to a mixture of (4a′S,10a′R)-methyl 4a′-benzyl-3′,4′,4a′,10a′-tetrahydro-1′H-spiro[[1,3]dioxolane-2,2′-phenanthrene]-7′-carboxylate (98, R²=Benzyl) (0.653 g, 1.62 mmol), 2-methyl-pyridin-3-ylamine (0.228 g, 2.11 mmol), and toluene (16 mL) under a nitrogen atmosphere at about 0° C. After about 30 min, the ice bath was removed and the mixture was stirred at rt for about 1 h. Saturated aqueous NaHCO₃ (50 mL) was added. The mixture was extracted with EtOAc (2×25 mL). The combined organics were washed with saturated aqueous NaCl (50 mL), dried over Na₂SO₄, filtered, and concentrated under reduced pressure. The residue was purified on silica gel (80 g) using a gradient of 50-100% EtOAc in DCM. The product containing fractions were combined and concentrated under reduced pressure to afford (4a′S,10aR)-4a′-benzyl-N-(2-methylpyridin-3-yl)-3′,4′,4a′,10a′-tetrahydro-1′H-spiro[[1,3]dioxolane-2,2′-phenanthrene]-7′-carboxamide (99, R²=Benzyl, R⁶=2-Methylpyridin-3-yl) (0.716 g, 95%) as a light tan foam. LC/MS, method 2, Rₜ=2.32 min, MS m/z 467 (M+H)⁺. ¹H NMR (400 MHz, DMSO-d₆) δ 10.01 (s, 1H), 8.34 (dd, J=4.8, 1.6 Hz, 1H), 7.82 (d, J=1.8 Hz, 1H), 7.79-7.73 (m, 2H), 7.28 (dd, J=8.0, 4.8 Hz, 1H), 7.24-7.17 (m, 3H), 7.15 (d, J=8.1 Hz, 1H), 6.86-6.80 (m, 2H), 6.70 (d, J=9.6 Hz, 1H), 6.22 (dd, J=9.5, 6.2 Hz, 1H), 3.83-3.71 (m, 4H), 2.74 (d, J=12.8 Hz, 1H), 2.63 (d, J=12.8 Hz, 1H), 2.46 (s, 3H), 2.38-2.31 (m, 1H), 2.31-2.21 (m, 1H), 1.79-1.68 (m, 1H), 1.66-1.54 (m, 2H), 1.47-1.36 (m, 1H), 0.99-0.91 (, m, 1H).

### Step #9: (4aS,11bS)-11b-Benzyl-6-methyl-N-(2-methylpyridin-3-yl)-1,2,4,4a,5,6,7,11b-octahydrospiro[dibenzo[c,e]azepine-3,2′-[1,3]dioxolane]-9-carboxamide (100, R²=Benzyl, R⁶=2-Methylpyridin-3-yl)

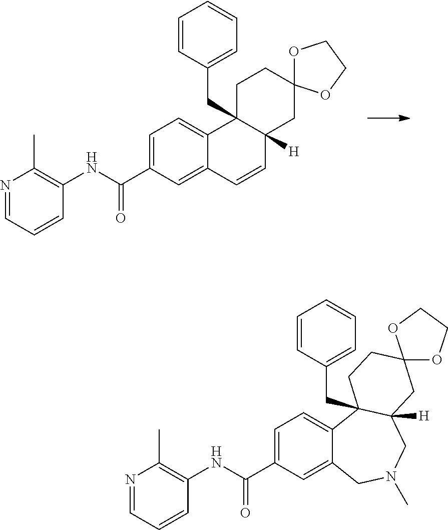

A solution of (4a′S,10a′R)-4a′-benzyl-N-(2-methylpyridin-3-yl)-3′,4′,4a′,10a′-tetrahydro-1′H-spiro[[1,3]dioxolane-2,2′-phenanthrene]-7′-carboxamide (99, R²=Benzyl, R⁶=2-Methylpyridin-3-yl) (0.760 g, 1.53 mmol), DCM (27 mL), and MeOH (3 mL) was purged with O₂ at about −78° C. for about 5 min. Oxygen was bubbled through the solution (˜1.5 SLPM) through an Ozone Gas Generator. After about 6 min, the solution began to turn blue. The reaction solution was purged with O₂ for about 15 min. PS-PPh₃ (˜3 mmol/g, 2.6 g) was added. The cold bath was removed and the reaction vessel was allowed to warm to rt. After about 14 h, PS-PPh₃ (˜3 mmol/g, 1.8 g) was added. After about 1 h, the mixture was filtered rinsing with DCM. The volatiles were removed under reduced pressure and then dried under high vacuum for about 15 min. The residue was dissolved in MeCN (20 mL). Methylamine (2 M solution in THF, 1.50 mL, 4.50 mmol) was added. After about 10 min, sodium cyanoborohydride (0.481 g, 7.66 mmol) was added. After about 2 h, sodium cyanoborohydride (0.481 g, 7.66 mmol) was added. After about 1 h, sodium cyanoborohydride (0.481 g, 7.66 mmol) was added. After about 1 h, saturated aqueous NaHCO₃ (10 mL) and water (40 mL) were added. After vigorously stirring for about 1 h, the mixture was extracted with EtOAc (3×50 mL). The combined organics were washed with saturated aqueous NaCl, dried over Na₂SO₄, filtered, and concentrated under reduced pressure. The residue was purified on silica gel (80 g) using a gradient of 0-10% MeOH in DCM then ((1% 7 N NH₃ in MeOH) in 10% MeOH in DCM). The fractions containing product were combined and concentrated under reduced pressure to afford a partial boron complex of (4aS,11bS)-11b-benzyl-6-methyl-N-(2-methylpyridin-3-yl)-1,2,4,4a,5,6,7,11b-octahydrospiro [dibenzo[c,e]azepine-3,2′-[1,3]dioxolane]-9-carboxamide (100, R²=Benzyl, R⁶=2-Methylpyridin-3-yl) (0.228 g, 30%) as an ivory solid. LC/MS, method 3, Rₜ=1.53 min, MS m/z 499 (M+H)⁺. ¹H NMR (400 MHz, DMSO-d₆) δ 9.99 (s, 1H), 8.33 (d, J=6.2 Hz, 1H), 7.84 (s, 1H), 7.74 (d, J=7.7 Hz, 1H), 7.67-7.61 (m, 1H), 7.30-7.25 (m, 1H), 7.15-7.06 (m, 3H), 6.86-6.81 (m, 1H), 6.68-6.62 (m, 2H), 3.99-3.72 (m, 6H), 3.48 (d, J=13.6 Hz, 1H), 3.25-3.18 (m, 1H), 2.72-2.50 (m, 3H), 2.45 (s, 3H), 2.40 (s, 3H), 2.16-2.00 (m, 2H), 1.63-1.39 (m, 4H).

### Step #10: (4aS,11bS)-11b-Benzyl-6-methyl-N-(2-methylpyridin-3-yl)-3-oxo-2,3,4,4a,5,6,7,11b-octahydro-1H-dibenzo[c,e]azepine-9-carboxamide (101, R²=Benzyl, R⁶=2-Methylpyridin-3-yl) and (3S,4aS,11bS)-11b-benzyl-3-hydroxy-6-methyl-N-(2-methylpyridin-3-yl)-2,3,4,4a,5,6,7,11b-octahydro-1H-dibenzo[c,e]azepine-9-carboxamide (102, R²=Benzyl, R³=H, R⁶=2-Methylpyridin-3-yl)

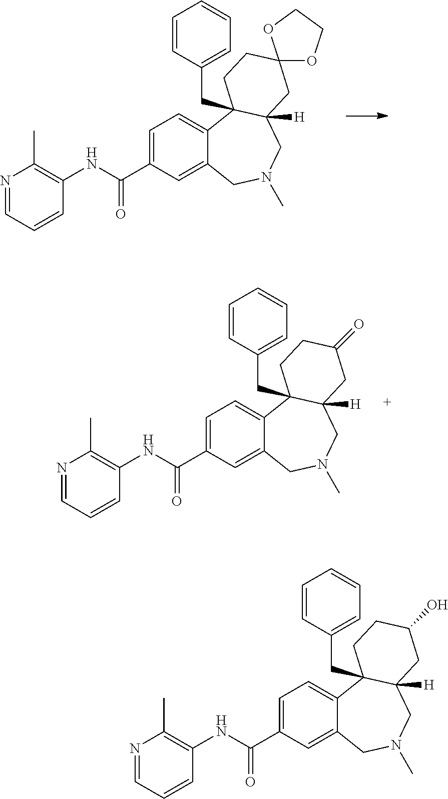

M Aqueous HCl (1.0 mL, 5.0 mmol) was slowly added to a solution of (4aS,11bS)-11b-benzyl-6-methyl-N-(2-methylpyridin-3-yl)-1,2,4,4 a,5,6,7,11b-octahydro spiro[dibenzo[c,e]azepine-3,2′-[1,3]dioxolane]-9-carboxamide (100, R²=Benzyl, R⁶=2-Methylpyridin-3-yl) (0.233 g, 0.393 mmol) as a partial boron complex and THF (6 mL) under air. The solution was left to stir for about 6 h. The solution was poured into saturated aqueous NaHCO₃ (10 mL). The mixture was extracted with EtOAc (3×10 mL). The combined organics were dried over Na₂SO₄, filtered, and concentrated under reduced pressure. The residue was purified on silica gel (25 g) using a gradient of 0-100% (1% 7 N NH₃ in MeOH) in 10% MeOH in DCM) in DCM. The fractions containing product were combined and concentrated under reduced pressure to afford (4aS,11bS)-11 b-benzyl-6-methyl-N-(2-methylpyridin-3-yl)-3-oxo-2,3,4,4a,5,6,7,11b-octahydro-1H-dibenzo[c,e]azepine-9-carboxamide (101, R²=Benzyl, R⁶=2-Methylpyridin-3-yl) (0.040 g, 22%) as an ivory solid and (3S,4aS,11bS)-11b-benzyl-3-hydroxy-6-methyl-N-(2-methylpyridin-3-yl)-2,3,4,4a,5,6,7,11b-octahydro-1H-dibenzo[c,e]azepine-9-carboxamide (102, R²=Benzyl, R⁶=2-Methylpyridin-3-yl) (0.088 g, 49%) as an ivory solid. (4aS,11bS)-11b-benzyl-6-methyl-N-(2-methylpyridin-3-yl)-3-oxo-2,3,4,4 a,5,6,7,11b-octahydro-1H-dibenzo[c,e]azepine-9-carboxamide (101, R²=Benzyl, R³=H, R⁶=2-Methylpyridin-3-yl): LC/MS, method 2, Rₜ=1.33 min, MS m/z 455 (M+H)⁺. ¹H NMR (400 MHz, DMSO-d₆) δ 10.03 (s, 1H), 8.34 (d, J=5.0 Hz, 1H), 7.91 (s, 1H), 7.74 (d, J=7.0 Hz, 1H), 7.68 (d, J=8.0 Hz, 1H), 7.28 (dd, J=7.9, 4.9 Hz, 1H), 7.15-7.06 (m, 3H), 6.92 (d, J=8.3 Hz, 1H), 6.66-6.58 (m, 2H), 4.04 (d, J=14.1 Hz, 1H), 3.83 (d, J=15.0 Hz, 1H), 3.64 (d, J=12.9 Hz, 1H), 3.21 (d, J=12.9 Hz, 1H), 2.74-2.64 (m, 2H), 2.44 (s, 3H), 2.42 (s, 3H), 2.38-2.17 (m, 5H), 2.08 (d, J=13.8 Hz, 1H), 1.90-1.78 (m, 1H). (3S,4aS,11bS)-11b-benzyl-3-hydroxy-6-methyl-N-(2-methylpyridin-3-yl)-2,3,4,4 a,5,6,7,11b-octahydro-1H-dibenzo[c,e]azepine-9-carboxamide (102, R²=Benzyl, R³=H, R⁶=2-Methylpyridin-3-yl): LC/MS, method 2, Rₜ=1.30 min, MS m/z 456 (M+H)⁺. ¹H NMR (400 MHz, DMSO-d₆) δ 9.98 (s, 1H), 8.38-8.27 (m, 1H), 7.84 (s, 1H), 7.74 (d, J=8.0 Hz, 1H), 7.64 (d, J=7.9 Hz, 1H), 7.28 (dd, J=7.9, 4.7 Hz, 1H), 7.14-7.03 (m, 3H), 6.80 (d, J=8.2 Hz, 1H), 6.66-6.53 (m, 2H), 4.35 (d, J=4.5 Hz, 1H), 3.95 (d, J=14.1 Hz, 1H), 3.78 (d, J=15.3 Hz, 1H), 3.57-3.45 (m, 2H), 3.24-3.15 (m, 1H), 2.73-2.63 (m, 1H), 2.60 (d, J=13.2 Hz, 1H), 2.45 (s, 3H), 2.41 (s, 3H), 2.17-2.07 (m, 1H), 1.80-1.65 (m, 2H), 1.62-1.53 (m, 1H), 1.46-1.33 (m, 1H), 1.29-1.13 (m, 2H).

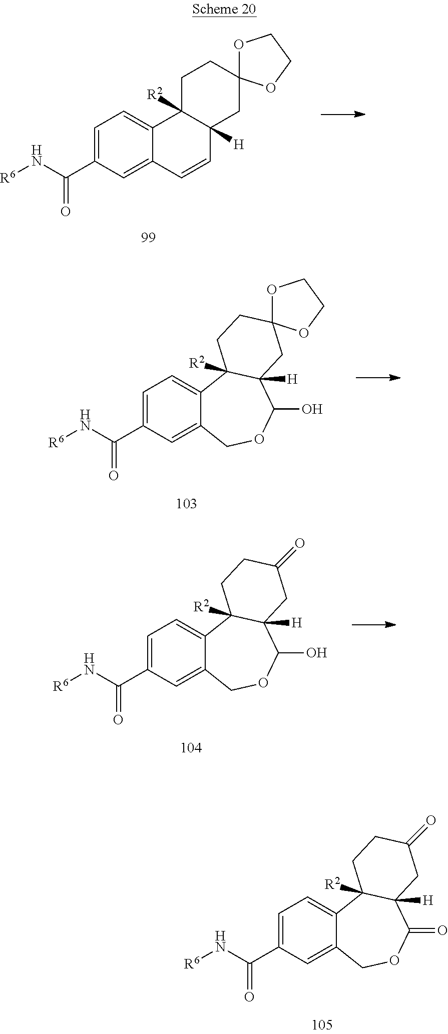

### Example #110

### (7aS,11aS)-11a-Benzyl-N-(2-methylpyridin-3-yl)-7,9-dioxo-5,7,7a,8,9,10,11,11a-octahydrodibenzo[c,e]oxepine-3-carboxamide (105, R²=Benzyl, R⁶=2-Methylpyridin-3-yl)

### Step #1: (4aS,11bS)-11b-Benzyl-5-hydroxy-N-(2-methylpyridin-3-yl)-2,4,4a,5,7,11b-hexahydro-1H-spiro[dibenzo[c,e]oxepine-3,2′-[1,3]dioxolane]-9-carboxamide (103, R²=Benzyl, R⁶=2-Methylpyridin-3-yl)

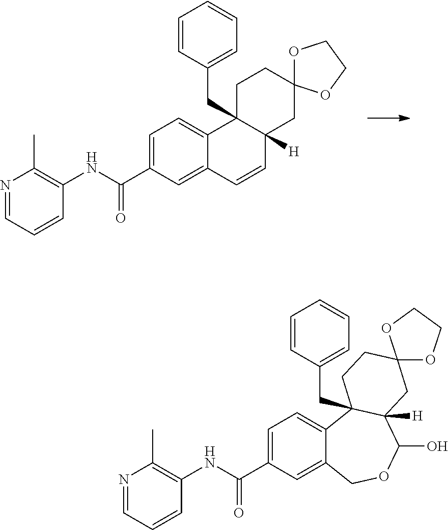

A solution of (4a′S,10a′R)-4a′-benzyl-N-(2-methylpyridin-3-yl)-3′,4′,4a′,10a′-tetrahydro-1′H-spiro[[1,3]dioxolane-2,2′-phenanthrene]-7′-carboxamide (99, R²=Benzyl, R⁶=2-Methylpyridin-3-yl) (0.205 g, 0.422 mmol), DCM (7.2 mL), and MeOH (0.8 mL) was purged with O₂ at about −78° C. for about 5 min. Oxygen was bubbled through the solution (˜1.5 SLPM) through an Ozone Gas Generator. After about 8 min, the solution began to turn blue. The reaction solution was purged with O₂ for about 15 min. PS-PPh₃ (˜3 mmol/g, 0.70 g) was added. The cold bath was allowed to thaw to rt over about 1 h. After about 2 h, the mixture was filtered rinsing with 50% MeOH in DCM (5 mL). NaBH₄ (0.048 g, 1.3 mmol) was added to the solution. After about 1 h, NaBH₄ (0.048 g, 1.3 mmol) was added. After about 1 h, the volatiles were removed under reduced pressure. Water (10 mL) and DCM (10 mL) were added. After vigorously stirring for about 15 min, the layers were separated. The aqueous phase was extracted with DCM (4×10 mL). The combined organics were dried over Na₂SO₄, filtered, and concentrated under reduced pressure. The residue was purified on silica gel (12 g) using a gradient of 0-15% MeOH in DCM. The fractions containing product were combined and concentrated under reduced pressure to afford an approximately 85:15 mixture of lactols, (4aS,11bS)-11b-benzyl-5-hydroxy-N-(2-methylpyridin-3-yl)-2,4,4a,5,7,11b-hexahydro-1H-spiro[dibenzo[c,e]oxepine-3,2′-[1,3]dioxolane]-9-carboxamide, (103, R²=Benzyl, R⁶=2-Methylpyridin-3-yl) (0.120 g, 57%) as a white solid. LC/MS, method 3, Major Isomer: Rₜ=1.87 min, MS m/z 501 (M+H)⁺. Minor Isomer: 1.81 min, MH⁺=501, Major Isomer: ¹H NMR (400 MHz, DMSO-d₆) δ 10.00 (s, 1H), 8.34 (dd, J=4.8, 1.6 Hz, 1H), 7.88 (d, J=2.1 Hz, 1H), 7.75 (dd, J=8.0, 1.5 Hz, 1H), 7.70 (dd, J=8.2, 2.0 Hz, 1H), 7.28 (dd, J=7.9, 4.8 Hz, 1H), 7.17-7.09 (m, 3H), 6.88 (d, J=8.5 Hz, 1H), 6.73-6.66 (m, 3H), 5.59-5.54 (m, 1H), 5.03 (d, J=14.8 Hz, 1H), 4.84 (d, J=15.2 Hz, 1H), 3.88-3.73 (m, 4H), 3.35 (d, J=13.1 Hz, 1H), 2.85 (d, J=13.1 Hz, 1H), 2.45 (s, 3H), 2.23-2.11 (m, 2H), 2.03-1.95 (m, 1H), 1.68-1.49 (m, 2H), 1.48-1.33 (m, 1H), 1.03-0.93 (m, 1H).

### Step #2: (7aS,11aS)-11a-Benzyl-7-hydroxy-9-oxo-5,7,7a,8,9,10,11,11a-octahydro-dibenzo[c,e]oxepine-3-carboxylic acid (2-methyl-pyridin-3-yl)-amide (104, R²=Benzyl, R⁶=2-Methylpyridin-3-yl)

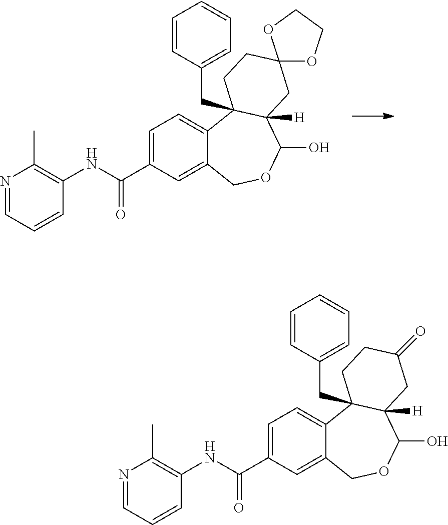

5 M aqueous HCl (0.4 mL, 2 mmol) was added dropwise to a solution of (4aS,11bS)-11b-benzyl-5-hydroxy-N-(2-methylpyridin-3-yl)-2,4,4a,5,7,11b-hexahydro-1H-spiro[dibenzo[c,e]oxepine-3,2′-[1,3]dioxolane]-9-carboxamide (103, R²=Benzyl, R⁶=2-Methylpyridin-3-yl) (0.111 g, 0.200 mmol) and THF under air over about 2 min. After about 1 h, the solution was poured into saturated aqueous NaHCO₃ (10 mL). The mixture was extracted with DCM (3×10 mL). The combined organics were dried over Na₂SO₄, filtered, and concentrated under reduced pressure. The residue was purified on silica gel (12 g) using a gradient 0-7.5% MeOH in DCM. The fractions containing product were combined and concentrated under reduced pressure to afford an approximately 9:1 ratio of lactols, (7aS,11aS)-11a-benzyl-7-hydroxy-9-oxo-5,7,7a,8,9,10,11,11a-octahydro-dibenzo[c,e]oxepine-3-carboxylic acid (2-methyl-pyridin-3-yl)-amide, (104, R²=Benzyl, R⁶=2-Methylpyridin-3-yl) (0.0906 mg, 99%) as an ivory solid. LC/MS, method 3, Major Isomer: Rₜ=1.67 min, MS m/z 457 (M+H)⁺, Minor Isomer: 1.73 min, 457 (M+H)⁺. Major Isomer: ¹H NMR (400 MHz, DMSO-d₆) δ 10.04 (s, 1H), 8.34 (dd, J=4.8, 1.6 Hz, 1H), 7.95 (d, J=2.0 Hz, 1H), 7.75 (dd, J=7.9, 1.4 Hz, 2H), 7.28 (dd, J=7.9, 4.7 Hz, 1H), 7.20-7.03 (m, 3H), 6.97 (d, J=8.5 Hz, 1H), 6.88 (d, J=4.3 Hz, 1H), 6.68-6.60 (m, 2H), 5.65-5.59 (m, 1H), 5.17 (d, J=14.7 Hz, 1H), 4.88 (d, J=15.0 Hz, 1H), 3.55 (d, J=13.0 Hz, 1H), 2.86 (d, J=13.2 Hz, 1H), 2.45 (s, 3H), 2.55-2.13 (m, 5H), 1.96-1.79 (m, 2H).

### Step #3: (7aS,11aS)-11a-Benzyl-N-(2-methylpyridin-3-yl)-7,9-dioxo-5,7,7a,8,9,10,11,11a-octahydrodibenzo[c,e]oxepine-3-carboxamide (105, R²=Benzyl, R⁶=2-Methylpyridin-3-yl)

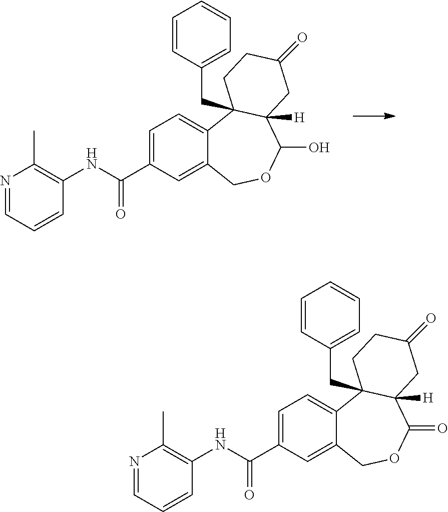

Crushed 4 Å molecular sieves (0.045 g) were added to a solution of (7aS,11aS)-11a-Benzyl-7-hydroxy-9-oxo-5,7,7a,8,9,10,11,11a-octahydro-dibenzo[c,e]oxepine-3-carboxylic acid (2-methyl-pyridin-3-yl)-amide (104, R²=Benzyl, R⁶=2-Methylpyridin-3-yl) (0.0218 g, 0.0480 mmol) and DCM (0.6 mL) under a nitrogen atmosphere. TPAP (0.0030 g, 0.0085 mmol) and NMO (0.017 g, 0.14 mmol) were respectively added, each in one portion. After about 30 min, the reaction mixture was filtered through Celite® rinsing with DCM. The solution was purified on silica gel (12 g) using a gradient 1-7.5% MeOH in DCM. The fractions containing product were combined and concentrated under reduced pressure. The residue was dissolved in MeCN and two drops of water were added. The volatiles were removed under reduced pressure and the residue was dried under high vacuum for about 15 h to afford (7aS,11aS)-11a-benzyl-N-(2-methylpyridin-3-yl)-7,9-dioxo-5,7,7a,8,9,10,11,11a-octahydrodibenzo[c,e]oxepine-3-carboxamide (105, R²=Benzyl, R⁶=2-Methylpyridin-3-yl) (0.0126 g, 58%) as an ivory solid. LC/MS, method 2, Rₜ=1.69 min, MS m/z 455 (M+H)⁺. ¹H NMR (400 MHz, CDCl₃) δ 8.40 (d, J=3.8 Hz, 1H), 8.32 (d, J=8.1 Hz, 1H), 8.03 (d, J=6.8 Hz, 1H), 7.92 (s, 1H), 7.87 (d, J=8.4 Hz, 1H), 7.66 (d, J=1.7 Hz, 1H), 7.31-7.21 (m, 2H), 7.17 (t, J=7.4 Hz, 2H), 6.66 (d, J=7.4 Hz, 2H), 5.03 (d, J=13.9 Hz, 1H), 4.67 (d, J=14.2 Hz, 1H), 4.07-4.00 (m, 1H), 3.59 (d, J=14.1 Hz, 1H), 3.38 (d, J=14.2 Hz, 1H), 2.93-2.51 (m, 5H), 2.64 (s, 3H), 2.37-2.21 (m, 1H).

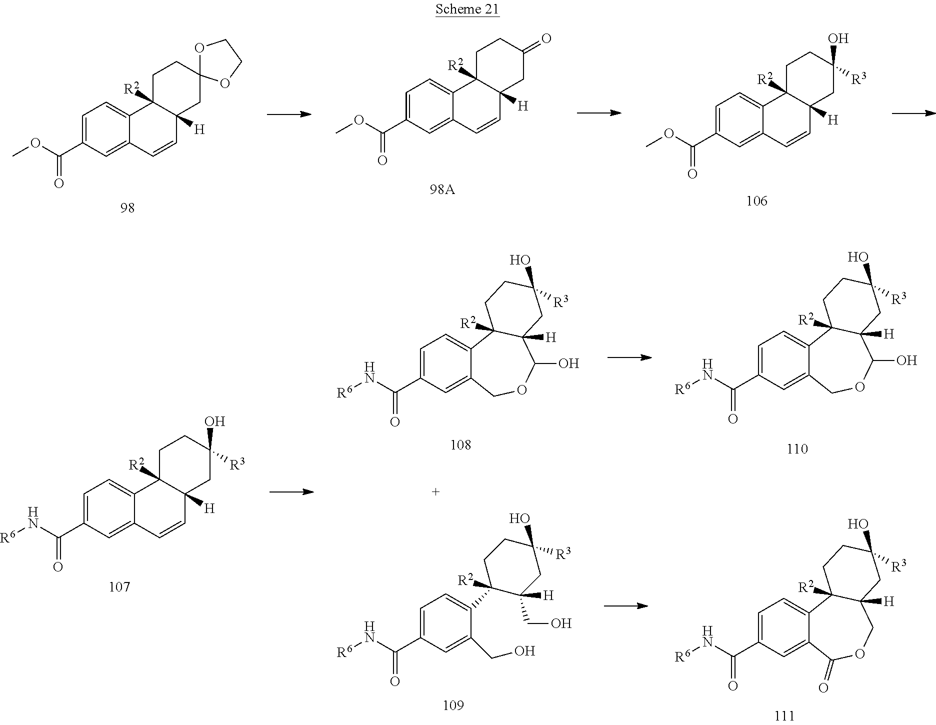

### Example #111

### (7aS,9R,11aS)-11a-Benzyl-9-ethyl-9-hydroxy-N-(2-methylpyridin-3-yl)-5,7,7a,8,9,10,11,11a-octahydrodibenzo[c,e]oxepine-3-carboxamide (110, R²=Benzyl, R³=Ethyl, R⁶=2-Methylpyridin-2-yl)

### Step #1: (4bS,8aR)-Methyl 4b-benzyl-7-oxo-4b,5,6,7,8,8a-hexahydrophenanthrene-2-carboxylate (98A, R²=Benzyl)

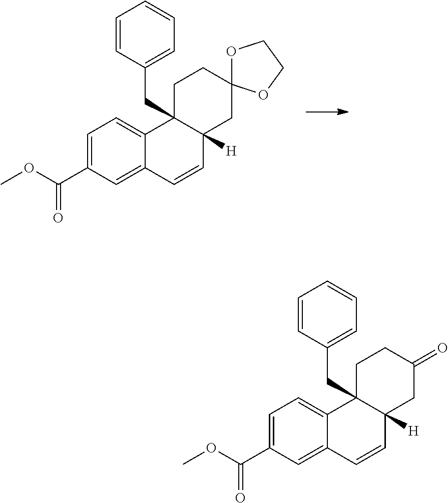

2 M aqueous HCl (10 mL, 20 mmol) was added to a solution of (4a′S,10a′R)-methyl 4a′-benzyl-3′,4′,4a′,10a′-tetrahydro-1′H-spiro[[1,3]dioxolane-2,2′-phenanthrene]-7′-carboxylate (98, R²=Benzyl) (1.74 g, 4.28 mmol) and THF (20 mL) under air. The biphasic mixture was left to vigorously stir for about 24 h. 6 M aqueous HCl (10 mL, 60 mmol) was added. After about 18 h, DCM (80 mL) was added. The layers were separated and the organics were washed with water (20 mL) and saturated aqueous NaCl (20 mL). The aqueous layers were extracted with DCM (20 mL). The combined organics were dried over Na₂SO₄, filtered, and concentrated under reduced pressure. The residue was purified on silica gel (120 g) using a gradient of 0-40% EtOAc in heptane. The fractions containing product were combined and concentrated under reduced pressure to afford (4bS,8aR)-methyl 4b-benzyl-7-oxo-4b,5,6,7,8,8a-hexahydrophenanthrene-2-carboxylate (98A, R²=Benzyl) (1.41 g, 95%) as an ivory foam. LC/MS, method 3, Rₜ=2.59 min, MS m/z 347 (M+H)⁺. ¹H NMR (400 MHz, DMSO-d₆) δ 7.86 (d, J=1.9 Hz, 1H), 7.76 (dd, J=8.0, 1.9 Hz, 1H), 7.20-7.11 (m, 4H), 6.76 (d, J=9.6 Hz, 1H), 6.75-6.69 (m, 2H), 6.19 (dd, J=9.5, 6.2 Hz, 1H), 3.87 (s, 3H), 2.90 (d, J=12.9 Hz, 1H), 2.64 (d, J=13.1 Hz, 1H), 2.68-2.27 (m, 3H), 2.21-2.06 (m, 2H), 1.95-1.81 m, 2H).

### Step #2: (4bS,7R,8aR)-Methyl 4b-benzyl-7-ethyl-7-hydroxy-4b,5,6,7,8,8a-hexahydrophenanthrene-2-carboxylate (106, R²=Benzyl, R³=Ethyl)

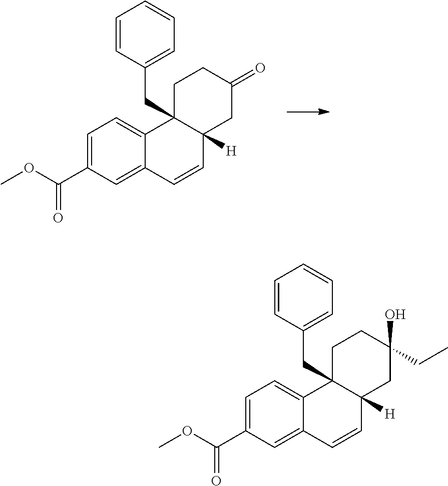

Ethylmagnesium bromide (3 M solution in Et₂O, 6.80 mL, 20.4 mmol) was added to THF (50 mL) under a nitrogen atmosphere. The solution was cooled to about −78° C. resulting in a light tan slurry. A solution of (4bS,8aR)-methyl 4b-benzyl-7-oxo-4b,5,6,7,8,8a-hexahydrophenanthrene-2-carboxylate (98A, R²=Benzyl) (1.43 g, 4.05 mmol) and THF (30 mL) was added dropwise maintaining an internal temperature of less than −60° C. The cold bath was thawed to between −40 and—50° C. over about 15 min and then maintained in this range for about 90 min. MeOH (1.5 mL) was added dropwise maintaining an internal temperature of less than −40° C. The cold bath was removed and saturated aqueous NH₄Cl (50 mL), water (50 mL), and EtOAc (100 mL) were added. The layers were separated and the organics were washed with saturated aqueous NaCl (50 mL). The aqueous layers were extracted with EtOAc (50 mL). The combined organics were dried over Na₂SO₄, filtered, and concentrated under reduced pressure. The residue was purified on silica gel (120 g) using a gradient of 10-30% EtOAc in heptane. The fractions containing product were combined and concentrated under reduced pressure to afford (4bS,7R,8aR)-methyl 4b-benzyl-7-ethyl-7-hydroxy-4b,5,6,7,8,8a-hexahydrophenanthrene-2-carboxylate (106, R²=Benzyl, R³=Ethyl) (1.19 g, 78%) as an ivory solid. LC/MS, method 3, Rₜ=2.71 min, MS m/z 377 (M+H)⁺. ¹H NMR (400 MHz, DMSO-d₆) δ 7.75 (d, J=1.8 Hz, 1H), 7.67 (dd, J=8.0, 1.8 Hz, 1H), 7.18-7.10 (m, 3H), 6.98 (d, J=8.1 Hz, 1H), 6.74-6.67 (m, 2H), 6.65 (d, J=9.5 Hz, 1H), 6.18 (dd, J=9.4, 6.2 Hz, 1H), 3.84 (s, 3H), 3.82 (s, 1H), 2.79 (d, J=12.8 Hz, 1H), 2.55 (d, J=12.8 Hz, 1H), 2.56-2.46 (m, 1H), 2.00-1.84 (m, 2H), 1.49-1.33 (m, 2H), 1.12 (q, J=7.4 Hz, 2H), 1.16-1.02 (m, 1H), 0.66 (t, J=7.4 Hz, 3H), 0.70-0.57 (m, 1H).

### Step #3: (4bS,7R,8aR)-4b-Benzyl-7-ethyl-7-hydroxy-N-(2-methylpyridin-3-yl)-4b,5,6,7,8,8 a-hexahydrophenanthrene-2-carboxamide (107, R²=Benzyl, R³=Ethyl, R⁶=2-Methylpyridin-3-yl)

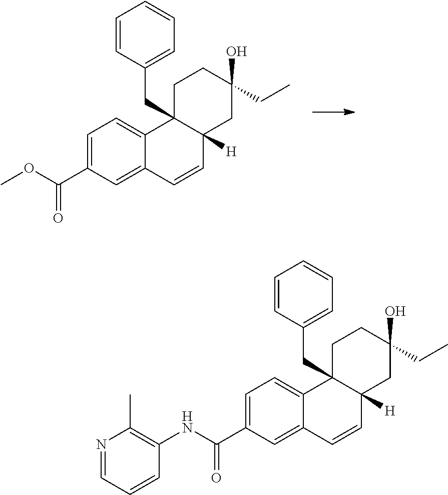

2-Methylpyridin-3-amine (0.113 g, 1.045 mmol) was added in one portion to a solution of (4bS,7R,8aR)-methyl 4b-benzyl-7-ethyl-7-hydroxy-4b,5,6,7,8,8 a-hexahydrophenanthrene-2-carboxylate (106, R²=Benzyl, R³=Ethyl) (0.302 g, 0.682 mmol) and toluene (8 mL) under a nitrogen atmosphere. The mixture was cooled to about 0° C. LiHMDS (1 M solution in THF, 3.0 mL, 3.0 mmol) was added dropwise over about 5 min. After about 30 min, the ice bath was removed. After about 15 min at rt, the mixture was poured into saturated aqueous NaHCO₃ (10 mL) and water (10 mL). The mixture was extracted with EtOAc (2×10 mL). The combined organics were dried over Na₂SO₄, filtered, and concentrated under reduced pressure. The residue was purified on silica gel (25 g) using a gradient of 50-100% EtOAc in DCM. The fractions containing product were combined and concentrated under reduced pressure to afford (4bS,7R,8aR)-4b-benzyl-7-ethyl-7-hydroxy-N-(2-methylpyridin-3-yl)-4b,5,6,7,8,8a-hexahydrophenanthrene-2-carboxamide (107, R²=Benzyl, R³=Ethyl, R⁶=2-Methylpyridin-3-yl) (0.252 g, 82%) as a pale yellow solid. LC/MS, method 3, Rₜ=2.18 min, MS m/z 454 (M+H)⁺. ¹H NMR (400 MHz, DMSO-d₆) δ 9.99 (s, 1H), 8.34 (dd, J=4.7, 1.6 Hz, 1H), 7.80-7.70 (m, 3H), 7.28 (dd, J=7.8, 4.7 Hz, 1H), 7.22-7.13 (m, 3H), 7.07 (d, J=8.1 Hz, 1H), 6.82-6.76 (m, 2H), 6.65 (d, J=9.5 Hz, 1H), 6.20 (dd, J=9.4, 6.2 Hz, 1H), 3.80 (s, 1H), 2.76 (d, J=12.8 Hz, 1H), 2.61 (d, J=12.9 Hz, 1H), 2.56-2.48 (m, 1H), 2.45 (s, 3H), 2.06-1.85 (m, 2H), 1.50-1.35 (m, 2H), 1.20-1.08 (m, 3H), 0.73-0.61 (m, 4H).

### Step #4: (7aS,9R,11aS)-11a-Benzyl-9-ethyl-7,9-dihydroxy-N-(2-methylpyridin-3-yl)-5,7,7a,8,9,10,11,11a-octahydrodibenzo[c,e]oxepine-3-carboxamide (108, R²=Benzyl, R³=Ethyl, R⁶=2-Methylpyridin-3-yl) and 4-((1S,2S,4R)-1-benzyl-4-ethyl-4-hydroxy-2-(hydroxymethyl)cyclohexyl)-3-(hydroxymethyl)-N-(2-methylpyridin-3-yl)benzamide (109, R²=Benzyl, R³=Ethyl, R⁶=2-Methylpyridin-3-yl)

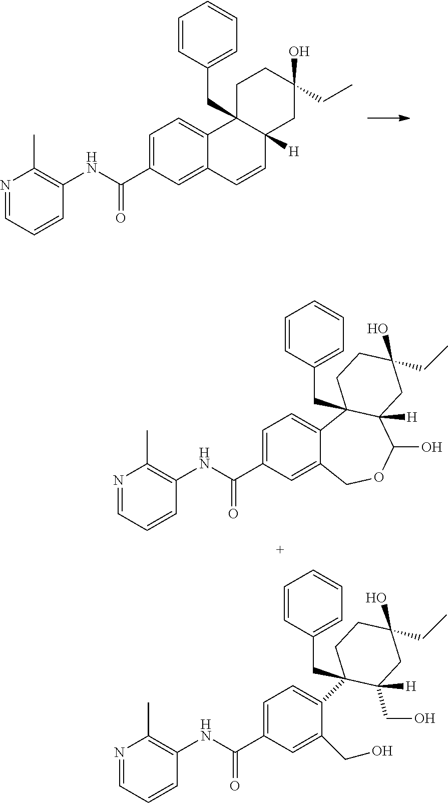

A solution of (4bS,7R,8aR)-4b-benzyl-7-ethyl-7-hydroxy-N-(2-methylpyridin-3-yl)-4b,5,6,7,8,8a-hexahydrophenanthrene-2-carboxamide (107, R²=Benzyl, R³=Ethyl, R⁶=2-Methylpyridin-3-yl) (0.280 g, 0.619 mmol), DCM (11 mL), and MeOH (1.2 mL) was purged with O₂ at about −78° C. for about 5 min. Oxygen was bubbled through the solution (˜2.0 SLPM) through an L11 Ozone Gas Generator from Pacific Ozone. After about 7 min, the solution began to turn blue. The ozone generator was switched off and the solution was purged with O₂ for about 15 min. PS-PPh₃ (˜3 mmol/g, 1.0 g) was added. The cold bath was allowed to thaw to rt over about 1 h. After about 90 min, the mixture was filtered rinsing with a solution of MeOH (5 mL) and DCM (5 mL). NaBH₄ (0.070 g, 1.9 mmol) was added. After about 1 h, NaBH₄ (0.023 g, 0.62 mmol) was added. After about 4 h, the volatiles were removed under reduced pressure. 5% MeOH in DCM (20 mL) and water (20 mL) were added. The mixture was left to vigorously stir for about 18 h. The layers were separated and the aqueous layer was extracted with 5% MeOH in DCM (2×10 mL). The combined organics were dried over Na₂SO₄, filtered, and concentrated under reduced pressure. The residue was purified on silica gel (25 g) using a gradient of 1-10% MeOH in DCM. The fractions containing product were combined and concentrated under reduced pressure to afford an approximately 9:1 mixture of lactols, (7aS,9R,11aS)-11a-benzyl-9-ethyl-7,9-dihydroxy-N-(2-methylpyridin-3-yl)-5,7,7a,8,9,10,11,11a-octahydrodibenzo[c,e]oxepine-3-carboxamide (108, R²=Benzyl, R³=Ethyl, R⁶=2-Methylpyridin-3-yl), (0.217 g, 72%) as an ivory solid and 4-((1S,2S,4R)-1-benzyl-4-ethyl-4-hydroxy-2-hydroxymethyl-cyclohexyl)-3-hydroxymethyl-N-(2-methyl-pyridin-3-yl)-benzamide (109, R²=Benzyl, R³=Ethyl, R⁶=2-Methylpyridin-3-yl) (0.026 g, 9%) as an ivory solid.

(7aS,9R,11aS)-11a-Benzyl-9-ethyl-7,9-dihydroxy-N-(2-methylpyridin-3-yl)-5,7,7a,8,9,10,11,11a-octahydrodibenzo[c,e]oxepine-3-carboxamide (108, R²=Benzyl, R³=Ethyl, R⁶=2-Methylpyridin-3-yl) Major isomer: LC/MS, method 3, Rₜ=1.75 min, MS m/z 488 (M+H)⁺. Minor isomer: LC/MS, method 3, Rₜ=1.78 min, MS m/z 488 (M+H)⁺, Major isomer: ¹H NMR (400 MHz, DMSO-d₆) δ 9.99 (s, 1H), 8.34 (dd, J=4.8, 1.6 Hz, 1H), 7.85 (d, J=2.0 Hz, 1H), 7.74 (dd, J=8.0, 1.5 Hz, 1H), 7.67 (dd, J=8.3, 1.9 Hz, 1H), 7.28 (dd, J=7.9, 4.7 Hz, 1H), 7.15-7.05 (m, 3H), 6.85 (d, J=8.5 Hz, 1H), 6.69-6.63 (m, 2H), 6.55 (d, J=4.3 Hz, 1H), 5.62-5.57 (m, 1H), 5.01 (d, J=14.8 Hz, 1H), 4.83 (d, J=14.9 Hz, 1H), 3.78 (s, 1H), 3.36 (d, J=13.1 Hz, 1H), 2.82 (d, J=12.9 Hz, 1H), 2.45 (s, 3H), 2.43-2.34 (m, 1H), 2.00-1.72 (m, 3H), 1.42-1.32 (m, 1H), 1.22-1.08 (m, 3H), 0.80-1.70 (m, 1H), 0.69 (t, J=7.4 Hz, 3H).

4-((1S,2S,4R)-1-Benzyl-4-ethyl-4-hydroxy-2-hydroxymethyl-cyclohexyl)-3-hydroxymethyl-N-(2-methyl-pyridin-3-yl)-benzamide (109, R²=Benzyl, R³=Ethyl, R⁶=2-Methylpyridin-3-yl): LC/MS, method 3, Rₜ=1.57 min, MS m/z 490 (M+H)⁺. ¹H NMR (400 MHz, DMSO-d₆) δ 9.99 (s, 1H), 8.33 (dd, J=4.8, 1.6 Hz, 1H), 8.26 (d, J=2.1 Hz, 1H), 7.74 (dd, J=8.0, 1.6 Hz, 1H), 7.70-7.62 (m, 1H), 7.27 (dd, J=8.0, 4.8 Hz, 1H), 7.11-7.05 (m, 3H), 7.04-6.99 (m, 1H), 6.87-6.77 (m, 2H), 5.43 (t, J=5.2 Hz, 1H), 5.08-4.97 (m, 1H), 4.77 (dd, J=13.6, 5.2 Hz, 1H), 4.43-4.36 (m, 1H), 3.96 (s, 1H), 3.43 (d, J=13.2 Hz, 1H), 3.26-3.14 (m, 2H), 2.44 (s, 3H), 2.43-2.34 (m, 1H), 2.06-1.97 (m, 1H), 1.92-1.71 (m, 3H), 1.61-1.52 (m, 1H), 1.51-1.28 (m, 3H), 0.84 (t, J=7.3 Hz, 3H).

### Step #5: (7aS,9R,11aS)-11a-Benzyl-9-ethyl-9-hydroxy-N-(2-methylpyridin-3-yl)-5,7,7a,8,9,10,11,11a-octahydrodibenzo[c,e]oxepine-3-carb oxamide (110, R²=Benzyl, R³=Ethyl, R⁶=2-Methylpyridin-3-yl)

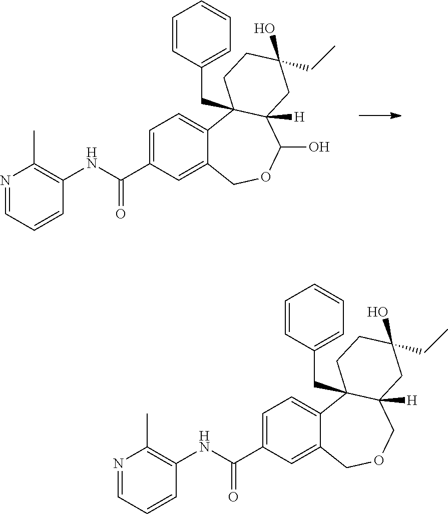

Trifluoroacetic acid (0.030 mL, 0.389 mmol) was added to a solution of (7aS,9R,11aS)-11a-benzyl-9-ethyl-7,9-dihydroxy-N-(2-methylpyridin-3-yl)-5,7,7a,8,9,10,11,11a-octahydrodibenzo[c,e]oxepine-3-carboxamide (108, R²=Benzyl, R³=Ethyl, R⁶=2-Methylpyridin-3-yl) (0.040 g, 0.082 mmol) and DCM (0.800 mL) under a nitrogen atmosphere at about 0° C. Triethylsilane (0.050 mL, 0.31 mmol) was added dropwise. The ice bath was removed and the solution was left to stir at rt. After about 20 h, DCM (0.800 mL) and triethylsilane (0.050 mL, 0.31 mmol) were added. After about 3 h, triethylsilane (0.050 mL, 0.31 mmol) was added. After about 2 h, the solution was poured into saturated aqueous NaHCO₃ (5 mL) and then extracted with DCM (4×5 mL). The combined organics were dried over Na₂SO₄, filtered, and concentrated under reduced pressure. The residue was purified on silica gel (12 g) using a gradient of 1-10% MeOH in DCM. The fractions containing product were combined and concentrated under reduced pressure. The residue was dissolved in MeCN and then water (4 mL) was added. The organic volatiles were removed under reduced pressure. The aqueous mixture was lyophilized to afford (7aS,9R,11aS)-11a-benzyl-9-ethyl-9-hydroxy-N-(2-methylpyridin-3-yl)-5,7,7a,8,9,10,11,11a-octahydrodibenzo[c,e]oxepine-3-carboxamide (110, R²=Benzyl, R³=Ethyl, R⁶=2-Methylpyridin-3-yl) (0.0172 g, 45%) as a white powder. LC/MS, method 2, Rₜ=1.94 min, MS m/z 472 (M+H)⁺. ¹H NMR (400 MHz, DMSO-d₆) δ 9.99 (s, 1H), 8.34 (dd, J=4.7, 1.5 Hz, 1H), 7.85 (d, J=1.9 Hz, 1H), 7.74 (dd, J=8.0, 1.4 Hz, 1H), 7.69-7.64 (m, 1H), 7.27 (dd, J=7.9, 4.8 Hz, 1H), 7.14-7.08 (m, 3H), 6.87 (d, J=8.5 Hz, 1H), 6.67-6.61 (m, 2H), 5.09 (d, J=14.3 Hz, 1H), 4.82 (d, J=14.6 Hz, 1H), 4.49 (d, J=12.2 Hz, 1H), 3.91 (s, 1H), 3.77 (dd, J=10.7, 2.2 Hz, 1H), 3.49 (d, J=13.0 Hz, 1H), 2.75 (d, J=13.1 Hz, 1H), 2.45 (s, 3H), 2.22-2.13 (m, 1H), 2.01-1.91 (m, 1H), 1.87-1.75 (m, 1H), 1.46-1.37 (m, 1H), 1.36-1.10 (m, 5H), 0.70 (t, J=7.4 Hz, 3H).

### Example #112

### (7aS,9R,11aS)-11a-Benzyl-9-ethyl-9-hydroxy-N-(2-methylpyridin-3-yl)-5-oxo-5,7,7a,8,9,10,11,11a-octahydrodibenzo[c,e]oxepine-3-carboxamide (111, R²=Benzyl, R³=Ethyl, R⁶=2-Methylpyridin-3-yl)

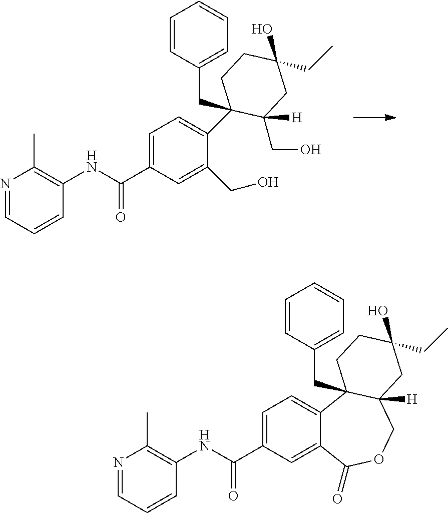

Manganese dioxide (88 mg, 1.0 mmol) was added to a solution containing 44(1S,2S,4R)-1-benzyl-4-ethyl-4-hydroxy-2-(hydroxymethyl)cyclohexyl)-3-(hydroxymethyl)-N-(2-methylpyridin-3-yl)benzamide (109, R²=Benzyl, R³=Ethyl, R⁶=2-Methylpyridin-3-yl) (25 mg, 0.051 mmol), DCM (4 mL) and THF (0.4 mL). The reaction was stirred at rt for about 18 h. The reaction was filtered through Celite® and washed with 10% MeOH in DCM (20 mL). The filtrate was concentrated under reduced pressure. The residue was purified on silica gel (4 g) using a gradient of 10-95% EtOAc in DCM. The fractions containing product were collected, combined and concentrated under reduced pressure to give an oil (24 mg) which was lyophilized to yield (7aS,9R,11aS)-11a-benzyl-9-ethyl-9-hydroxy-N-(2-methylpyridin-3-yl)-5-oxo-5,7,7a,8,9,10,11,11a-octahydrodibenzo[c,e]oxepine-3-carboxamide (111, R²=Benzyl, R³=Ethyl, R⁶=2-Methylpyridin-3-yl) (15 mg, 60%) as a solid. LC/MS, method 2, Rₜ=1.90 min, MS m/z 485 (M+H)⁺. ¹H NMR (400 MHz, DMSO-d₆) δ 10.26 (s, 1H), 8.40-8.31 (m, 2H), 8.09-8.04 (m 1H), 7.78-7.73 (m, 1H), 7.34-7.26 (m, 2H), 7.12-7.02 (m, 3H), 6.74-6.66 (m, 2H), 4.41-4.31 (m, 1H), 4.11 (s, 1H), 3.71 (dd, J=12.2, 12.2 Hz, 1H), 3.37 (d, J=13.7 Hz, 1H), 2.82-2.72 (m, 2H), 2.45 (s, 3H), 2.15-2.05 (m, 1H), 1.92-1.82 (m, 1H), 1.62-1.40 (m, 2H), 1.36-1.20 (m, 3H), 0.79 (t, J=7.3 Hz, 3H), 0.52-0.41 (m 1H).

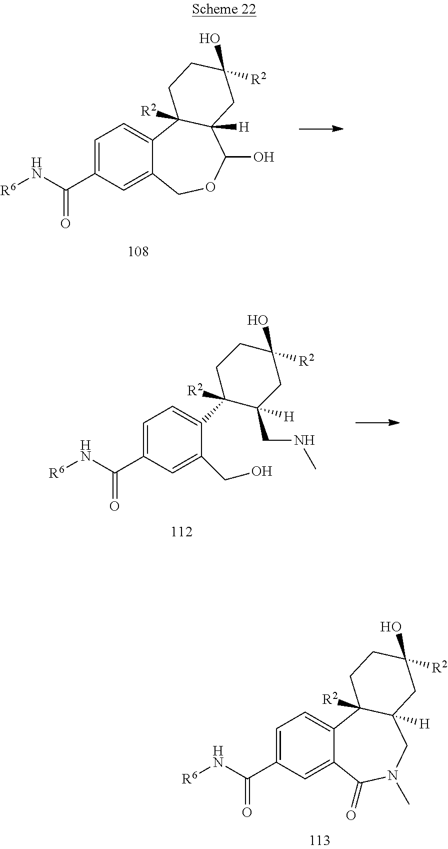

### Example #113

### (3R,4aR,11bS)-11b-Benzyl-3-ethyl-3-hydroxy-6-methyl-N-(2-methylpyridin-3-yl)-7-oxo-2,3,4,4a,5,6,7,11b-octahydro-1H-dibenzo[c,e]azepine-9-carboxamide (113, R²=Benzyl, R³=Ethyl, R⁶=2-Methylpyridin-3-yl)

### Step #1: 4-((1S,2R,4R)-1-Benzyl-4-ethyl-4-hydroxy-2-((methylamino)methyl)cyclohexyl)-3-(hydroxymethyl)-N-(2-methylpyridin-3-yl)benzamide (112, R²=Benzyl, R³=Ethyl, R⁶=2-Methylpyridin-3-yl)

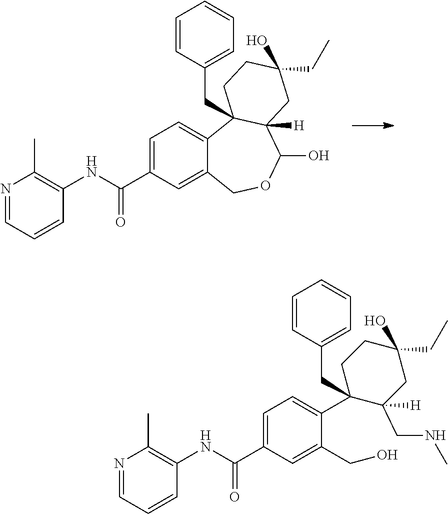

Methylamine hydrochloride (0.620 g, 9.18 mmol) and sodium cyanoborohydride (0.100 g, 1.59 mmol) were added respectively, each in one portion, to a solution of (7aS,9R,11aS)-11a-benzyl-9-ethyl-7,9-dihydroxy-N-(2-methylpyridin-3-yl)-5,7,7a,8,9,10,11,11a-octahydrodibenzo[c,e]oxepine-3-carboxamide (108, R²=Benzyl, R³=Ethyl, R⁶=2-Methylpyridin-3-yl) (0.152 g, 0.306 mmol), EtOH (2.50 mL), and AcOH (0.500 mL). The system was sealed and the reaction vessel was warmed to about 90° C. After about 3 days, the mixture was allowed to cool to rt. Sodium cyanoborohydride (0.100 g, 1.59 mmol) and methylamine hydrochloride (0.310 g, 4.59 mmol) were added. The reaction vessel was sealed and the mixture was warmed to about 90° C. After about 4 days, the mixture was allowed to cool to rt. Water (4 mL), saturated aqueous NH₄Cl (1 mL), and DCM (10 mL) were added. The mixture was left to vigorously stir for about 3 h. The aqueous layer was made basic with saturated aqueous NaHCO₃. The layers were separated and the aqueous phase was extracted with DCM (4×10 mL). The combined organics were dried over Na₂SO₄, filtered, and concentrated under reduced pressure. The residue was purified on silica gel (12 g) using a gradient of 10-100% ((2% NH₄OH) in 20% MeOH in DCM) in DCM then hold at (2% NH₄OH) in 20% MeOH in DCM. The fractions containing product were combined and concentrated under reduced pressure to afford 4-((1S,2R,4R)-1-benzyl-4-ethyl-4-hydroxy-2-((methylamino)methyl)cyclohexyl)-3-(hydroxymethyl)-N-(2-methylpyridin-3-yObenzamide (112, R²=Benzyl, R³=Ethyl, R⁶=2-Methylpyridin-3-yl) (0.0405, 26%) as a white solid. LC/MS, method 3, Rₜ=1.55 min, MS m/z 503 (M+H)⁺. ¹H NMR (400 MHz, DMSO-d₆) δ 9.96 (s, 1H), 8.33 (dd, J=4.8, 1.6 Hz, 1H), 8.22 (d, J=2.2 Hz, 1H), 7.73 (dd, J=7.9, 1.6 Hz, 1H), 7.59-7.52 (m, 1H), 7.27 (dd, J=8.0, 4.8 Hz, 1H), 7.06-6.95 (m, 3H), 6.93-6.79 (m, 1H), 6.67-6.59 (m, 2H), 5.46-5.37 (m, 1H), 4.99-4.88 (m, 1H), 4.85-4.74 (m, 1H), 3.30-3.22 (m, 1H), 3.19-3.06 (m, 1H), 2.90-2.72 (m, 3H), 2.43 (s, 3H), 2.35 (s, 3H), 2.17-2.05 (m, 1H), 1.98-1.86 (m, 1H), 1.67-1.58 (m, 1H), 1.51-1.42 (m, 1H), 1.39-1.25 (m, 1H), 1.17-1.06 (m, 2H), 1.00-0.89 (m, 1H), 0.70 (t, J=7.4 Hz, 3H).

### Step #2: (3R,4aR,11bS)-11b-Benzyl-3-ethyl-3-hydroxy-6-methyl-N-(2-methylpyridin-3-yl)-7-oxo-2,3,4,4a,5,6,7,11b-octahydro-1H-dibenzo[c,e]azepine-9-carboxamide (113, R²=Benzyl, R³=Ethyl, R⁶=2-Methylpyridin-3-yl)

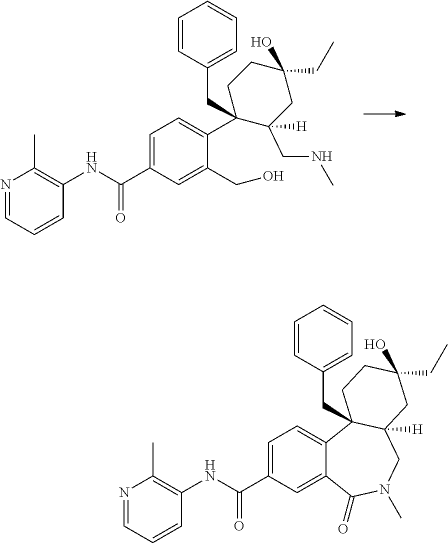

TPAP (0.0030 g, 0.0085 mmol) was added in one portion to a mixture of 4-((1S,2S,4R)-1-benzyl-4-ethyl-4-hydroxy-2-((methylamino)methyl)cyclohexyl)-3-(hydroxymethyl)-N-(2-methylpyridin-3-yl)benzamide (112, R²=Benzyl, R³=Ethyl, R⁶=2-Methylpyridin-3-yl) (0.040 g, 0.077 mmol), crushed 4 Å molecular sieves (0.120 g), and DCM (1.50 mL) under a nitrogen atmosphere. NMO (0.054 g, 0.464 mmol) was added in one portion. After about 15 h, the mixture was filtered through Celite® rinsing with DCM (3×5 mL). The organics were concentrated to about 1 mL under reduced pressure. The solution was purified on silica gel (12 g) using a gradient of 2-10% MeOH in DCM. The fractions containing product were combined and concentrated under reduced pressure. The residue was dissolved in MeCN and water (1 mL) was added. The organic volatiles were removed under reduced pressure. The mixture was lyophilized to afford (3R,4aR,11bS)-11b-benzyl-3-ethyl-3-hydroxy-6-methyl-N-(2-methylpyridin-3-yl)-7-oxo-2,3,4,4a,5,6,7,11b-octahydro-1H-dibenzo[c,e]azepine-9-carboxamide (113, R²=Benzyl, R³=Ethyl, R⁶=2-Methylpyridin-3-yl) (0.0096 g, 25%) as a fluffy white solid. LC/MS, method 2, Rₜ=1.72 min, MS m/z 499 (M+H)⁺. ¹H NMR (400 MHz, DMSO-d₆) δ 10.16 (s, 1H), 8.34 (dd, J=4.8, 1.6 Hz, 1H), 8.23 (d, J=2.1 Hz, 1H), 7.91 (dd, J=8.2, 2.1 Hz, 1H), 7.74 (dd, J=8.0, 1.5 Hz, 1H), 7.28 (dd, J=8.0, 4.7 Hz, 1H), 7.08 (d, J=8.4 Hz, 1H), 7.04-6.96 (m, 3H), 6.73-6.66 (m, 2H), 4.38 (s, 1H), 3.68 (dd, J=15.6, 7.8 Hz, 1H), 3.49 (d, J=13.9 Hz, 1H), 3.14 (s, 3H), 3.00 (d, J=15.3 Hz, 1H), 2.91 (d, J=14.0 Hz, 1H), 2.44 (s, 3H), 2.32-2.21 (m, 1H), 2.16-2.04 (m, 1H), 1.92-1.82 (m, 1H), 1.77-1.40 (m, 6H), 0.84 (t, J=7.3 Hz, 3H).

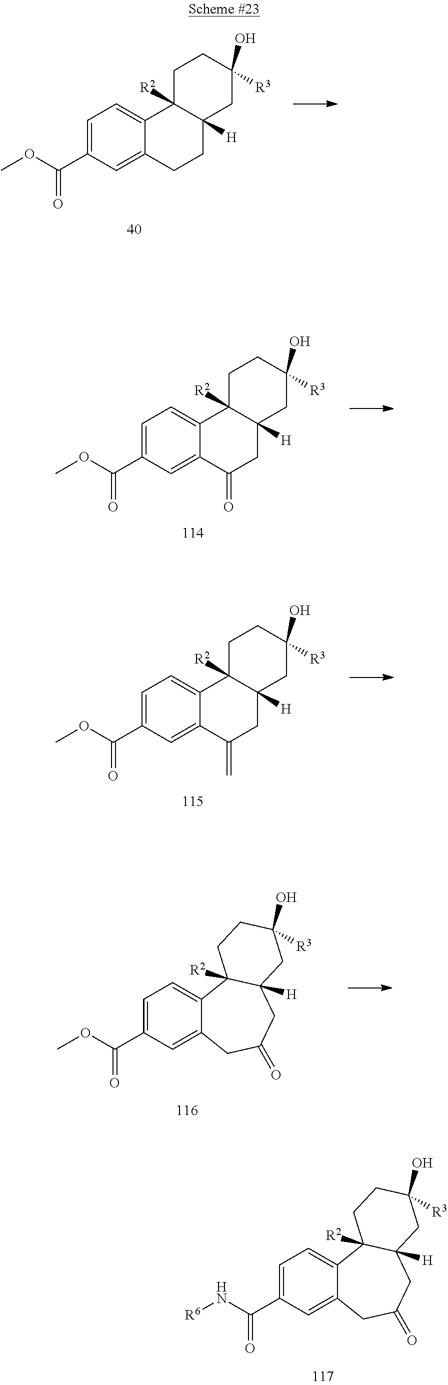

### Example #114

### (7aR,9R,11aS)-11a-Benzyl-9-ethyl-9-hydroxy-6-oxo-6,7,7a,8,9,10,11,11a-octahydro-5H-dibenzo[a,c]cycloheptene-3-carboxylic acid (2-methyl-pyridin-3-yl)-amide (117, R²=Benzyl, R³=Ethyl, R⁶=2-Methylpyridin-3-yl)

### Step #1: (4bS,7R,8aR)-Methyl 4b-benzyl-7-ethyl-7-hydroxy-10-oxo-4b,5,6,7,8,8a,9,10-octahydrophenanthrene-2-carboxylate (114, R²=Benzyl, R³=Ethyl)

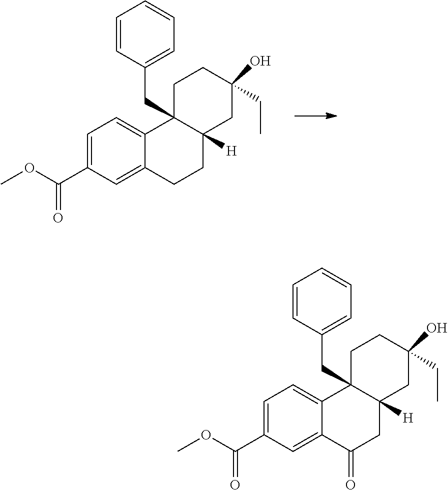

To a solution of (4bS,7R,8aS)-methyl 4b-benzyl-7-ethyl-7-hydroxy-4b,5,6,7,8,8a,9,10-octahydrophenanthrene-2-carboxylate (40, R²=Benzyl, R³=Ethyl) (6.60 g, 16.4 mmol) and DCM (150 mL) under air was added copper(II) sulfate pentahydrate (17.4 g, 69.5 mmol) and potassium permanganate (10.4 g, 65.6 mmol) respectively, each in one portion. Water (18 mL) and pyridine (5.7 mL, 71 mmol) were added sequentially. The mixture was vigorously stirred under air for about 40 h, then Na₂SO₄ (70 g) was added. The mixture was stirred for about 30 min and then filtered through Celite®, rinsing with DCM (10×30 mL). The filtrate was concentrated under reduced pressure. The residue was mixed in EtOAc (400 mL) and water (200 mL) for about 5 min then filtered through Celite® rinsing with EtOAc (100 mL). The layers were separated and the organic layer was washed with water (4×70 mL), 0.1 M aqueous EDTA tetrasodium salt solution (2×120 mL) and water (2×50 mL). The organic layer was dried over MgSO₄, filtered, and concentrated under reduced pressure. The residue was purified on silica gel (220 g) using a gradient of 0-20% EtOAc in DCM. The fractions containing product were collected, combined and concentrated under reduced pressure. The residue was purified again on silica gel (220 g) using a gradient of 0-8% EtOAc in DCM. The fractions containing product were collected, combined and concentrated to give a light yellow foam. The foam was dissolved in DCM (120 mL) and washed with 0.1 M EDTA (2×50 mL) then with water (50 mL). The aqueous layer was extracted with DCM (100 mL). The organic layer was dried over Na₂SO₄, filtered and concentrated to afford (4bS,7R,8aR)-methyl 4b-benzyl-7-ethyl-7-hydroxy-10-oxo-4b,5,6,7,8,8a,9,10-octahydrophenanthrene-2-carboxylate (114, R²=Benzyl, R³=Ethyl) (4.00 g, 62%) as white foam. LC/MS, method 3, Rₜ=2.33 min, MS m/z 393 (M+H)⁺. ¹H NMR (600 MHz, DMSO-d₆) δ 8.50 (d, J=2.0 Hz, 1H), 8.03 (dd, J=8.2, 2.0 Hz, 1H), 7.25-7.17 (m, 4H), 6.95-6.89 (m, 2H), 3.88 (s, 3H), 3.84 (s, 1H), 3.47 (dd, J=17.9, 5.3 Hz, 1H), 3.00 (d, J=13.3 Hz, 1H), 2.93 (d, J=13.2 Hz, 1H), 2.50-2.43 (m, 1H), 2.29 (dd, J=18.0, 1.6 Hz, 1H), 2.10-2.04 (m, 1H), 1.99-1.92 (m, 1H), 1.45-1.38 (m, 1H), 1.38-1.32 (m, 1H), 1.10-1.04 (m, 2H), 0.94-0.87 (m, 1H), 0.87-0.80 (m, 1H), 0.63 (t, J=7.5 Hz, 3H).

### Step #2: (4bS,7R,8aS)-Methyl 4b-benzyl-7-ethyl-7-hydroxy-10-methylene-4b,5,6,7,8,8a,9,10-octahydrophenanthrene-2-carboxylate (115, R²=Benzyl, R³=Ethyl)

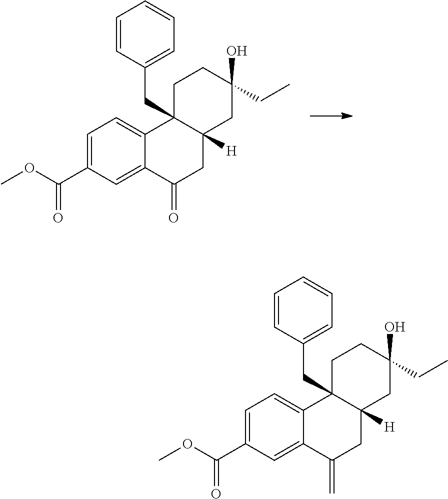

To a suspension of potassium tert-butoxide (672 mg, 5.99 mmol) in Et₂O (20 mL) was added methyltriphenylphosphonium bromide (2.27 g, 6.35 mmol). The reaction was vigorously stirred for about 20 min at rt. A solution of (4bS,7R,8aR)-methyl 4b-benzyl-7-ethyl-7-hydroxy-10-oxo-4b,5,6,7,8,8a,9,10-octahydrophenanthrene-2-carboxylate (114, R²=Benzyl, R³=Ethyl) (500 mg, 1.20 mmol) in Et₂O (20 mL) was added dropwise via syringe and the reaction was stirred at rt for about 4 h, then quenched with aqueous NH₄Cl (75 mL) and water (25 mL) and the mixture was extracted with EtOAc (150 mL). The organic layer was concentrated under reduced pressure. The crude material was purified on silica gel (120 g) using a gradient of 0-7% EtOAc in DCM. The fractions containing product were combined and concentrated to give (4bS,7R,8aS)-methyl 4b-benzyl-7-ethyl-7-hydroxy-10-methylene-4b,5,6,7,8,8a,9,10-octahydrophenanthrene-2-carboxylate (115, R²=Benzyl, R³=Ethyl) (310 mg, 66%) as white foam. LC/MS, method 3, Rₜ=2.70 min, No parent ion. ¹H NMR (400 MHz, DMSO-d₆) δ 8.31 (d, J=1.9 Hz, 1H), 7.73-7.68 (m, 1H), 7.25-7.15 (m, 3H), 7.09 (d, J=8.3 Hz, 1H), 6.93-6.88 (m, 2H), 5.74 (s, 1H), 5.12 (s, 1H), 3.86 (s, 3H), 3.74 (s, 1H), 3.32-3.20 (m, 1H), 2.83 (d, J=13.1 Hz, 1H), 2.73 (d, J=12.9 Hz, 1H), 2.25-2.15 (m, 2H), 2.04-1.83 (m, 2H), 1.34-1.22 (m, 2H), 1.12-1.02 (m, 2H), 0.93-0.75 (m, 2H), 0.63 (t, J=7.3 Hz, 3H).

### Step #3: (7aR,9R,11aS)-11a-Benzyl-9-ethyl-9-hydroxy-6-oxo-6,7,7a,8,9,10,11,11a-octahydro-5H-dibenzo[a,c]cycloheptene-3-carboxylic acid methyl ester (116, R²=Benzyl, R³=Ethyl)

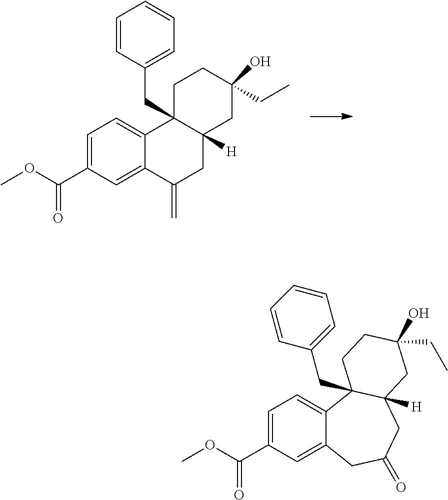

(4bS,7R,8aS)-Methyl 4b-benzyl-7-ethyl-7-hydroxy-10-methylene-4b,5,6,7,8,8 a,9,10-octahydrophenanthrene-2-carboxylate (115, R²=Benzyl, R³=Ethyl) (200 mg, 0.512 mmol) was dissolved in MeOH (19 mL) and water (0.2 mL). [hydroxy(tosyloxy)iodo]benzene (201 mg, 0.512 mmol) was added in one portion. The reaction was mixed at rt for about 18 h. The reaction was diluted with DCM (200 mL) and washed with saturated aqueous NaCl (2×20 mL). The organic layer was dried over Na₂SO₄, filtered and concentrated under reduced pressure. The crude material was purified on silica gel (40 g) using a gradient of 0-25% EtOAc in DCM. The fractions containing product were combined and concentrated under reduced pressure to give (7aR,9R,11aS)-11a-benzyl-9-ethyl-9-hydroxy-6-oxo-6,7,7a,8,9,10,11,11a-octahydro-5H-dibenzo[a,c]cycloheptene-3-carboxylic acid methyl ester (116, R²=Benzyl, R³=Ethyl) (60 mg, 29%) as colorless oil. LC/MS, method 3, Rₜ=2.26 min, MS m/z 405 (M−H)⁻. ¹H NMR (600 MHz, DMSO-d₆) δ 7.79 (s, 1H), 7.58 (d, J=8.4 Hz, 1H), 7.13-7.04 (m, 3H), 6.92 (d, J=8.5 Hz, 1H), 6.66-6.61 (m, 2H), 4.66 (d, J=13.1 Hz, 1H), 3.98 (s, 1H), 3.83 (s, 3H), 3.71 (d, J=13.2 Hz, 1H), 3.69-3.61 (m, 1H), 3.58 (d, J=13.1 Hz, 1H), 2.79 (d, J=13.1 Hz, 1H), 2.67-2.59 (m, 1H), 2.18-2.12 (m, 1H), 2.03-1.96 (m, 1H), 1.91-1.83 (m, 1H), 1.45-1.00 (m, 5H), 0.72-0.64 (m, 1H), 0.64 (t, J=7.5 Hz, 3H).

### Step #4: (7aR,9R,11aS)-11a-Benzyl-9-ethyl-9-hydroxy-6-oxo-6,7,7a,8,9,10,11,11a-octahydro-5H-dibenzo[a,c]cycloheptene-3-carboxylic acid (2-methyl-pyridin-3-yl)-amide (117, R²=Benzyl, R³=Ethyl, R⁶=2-Methylpyridin-3-yl)

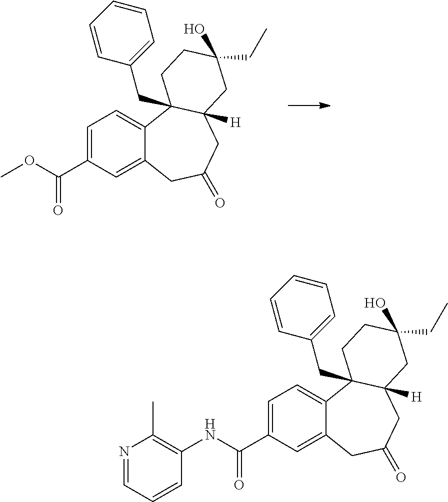

LiHMDS (1 M solution in THF, 0.300 mL, 0.300 mmol) was added dropwise to a solution of (7aR,9R,11aS)-11a-benzyl-9-ethyl-9-hydroxy-6-oxo-6,7,7a,8,9,10,11,11a-octahydro-5H-dibenzo[a,c]cycloheptene-3-carboxylic acid methyl ester (116, R²=Benzyl, R³=Ethyl) (0.023 g, 0.058 mmol) in toluene (0.5 mL) under a nitrogen atmosphere at about 0° C. for about 5 min. 2-Methylpyridin-3-amine (0.0080 g, 0.074 mmol) was added and the reaction was stirred for about 15 min at about 0° C. The ice bath was removed and the brown mixture was left to stir for about 3 h. Water (10 mL) and EtOAc (10 mL) were added. The layers were separated and the organic layer was washed with water (5 mL) and then saturated aqueous NaCl (5 mL), dried over Na₂SO₄, filtered, and concentrated under reduced pressure. The crude material was purified on silica gel (12 g) using a gradient from 50-100% EtOAc in DCM. The fractions containing product were combined and concentrated under reduced pressure. The residue was purified by HPLC: The gradient was 10% B for 2.5 min then 10-15% B in 1.0 min then 15-70% B in 9 min then 70-95% in 0.3 min then 95% for 0.7 min (22.5 mL/min flow rate). Mobile phase A: 50 mM NH₄OAc in water, mobile phase B was HPLC grade MeCN. The column used for chromatography is 19×50 mm Waters Atlantis T3 OBD C18 column (5.0 μm particles). Detection methods are photodiode array (DAD) and Waters ZQ 2000 mass spectrometer. The organic volatiles were removed under reduced pressure. The mixture was frozen then lyophilized to provide a white solid. The material was slurried in water (5 mL) and then lyophilized to yield (7aR,9R,11aS)-11a-benzyl-9-ethyl-9-hydroxy-6-oxo-6,7,7a,8,9,10,11,11a-octahydro-5H-dibenzo[a,c]cycloheptene-3-carboxylic acid (2-methyl-pyridin-3-yl)-amide (117, R²=Benzyl, R³=Ethyl, R⁶=2-Methylpyridin-3-yl) (0.0066 g, 24%) as a white solid. LC/MS, method 2, Rₜ=1.87 min, MS m/z 483 (M+H)⁺. ¹H NMR (400 MHz, DMSO-d₆) δ 10.01 (s, 1H), 8.36-8.30 (m, 1H), 7.82 (s, 1H), 7.72 (d, J=8.0 Hz, 1H), 7.62 (d, J=8.4 Hz, 1H), 7.26 (dd, J=8.0, 4.8 Hz, 1H), 7.15-7.06 (m, 3H), 6.94 (d, J=8.5 Hz, 1H), 6.74-6.66 (m, 2H), 4.70 (d, J=13.0 Hz, 1H), 3.98 (s, 1H), 3.74-3.62 (m, 2H), 3.60-3.51 (m, 1H), 2.81 (d, J=13.4 Hz, 1H), 2.68-2.56 (m, 1H), 2.43 (s, 3H), 2.19-2.12 (m, 1H), 2.06-1.97 (m, 1H), 1.93-1.80 (m, 1H), 1.48-1.38 (m, 1H), 1.32-1.21 (m, 2H), 1.13-1.03 (m, 2H), 0.76-0.69 (m, 1H), 0.65 (t, J=7.4 Hz, 3H).

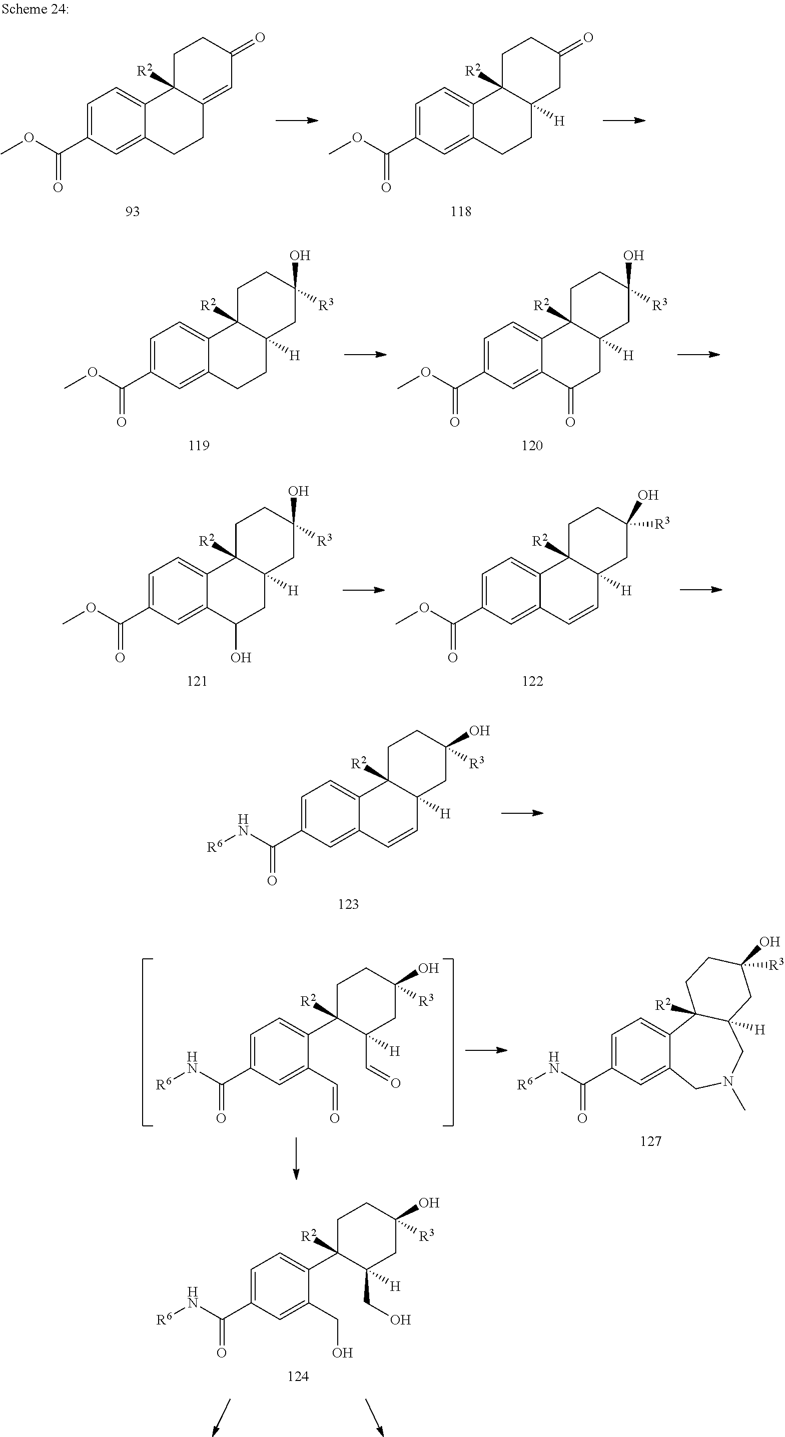

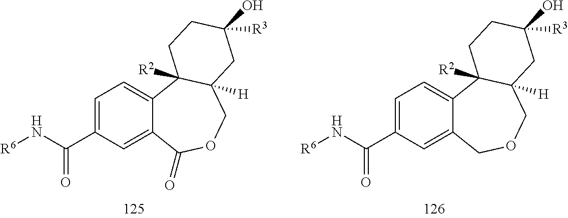

### Example #115

### (7aR,9R,11aS)-11a-Benzyl-9-hydroxy-N-(2-methylpyridin-3-yl)-5-oxo-9-(trifluoromethyl)-5,7,7a,8,9,10,11,11a-octahydrodibenzo[c,e]oxepine-3-carboxamide (125, R²=Benzyl, R³=Trifluoromethyl, R⁶=2-Methylpyridin-3-yl)

### Step #1: (4bS,8aR)-Methyl 4b-benzyl-7-oxo-4b,5,6,7,8,8a,9,10-octahydrophenanthrene-2-carboxylate (118, R²=Benzyl)

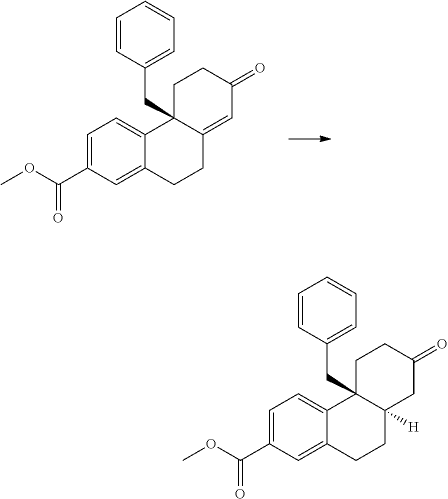

A solution of (S)-methyl 4b-benzyl-7-oxo-4b,5,6,7,9,10-hexahydrophenanthrene-2-carboxylate (93, R²=Benzyl) (28 g, 81 mmol), trichloroacetic acid (8.1 mL, 81 mmol), (2R,5R)-5-benzyl-3-methyl-2-(5-methylfuran-2-yl)imidazolidin-4-one (37.0 g, 24.3 mmol, 17.7 wt % in toluene) and toluene (78 mL) was stirred for about 1 h at rt. The reaction was then charged with diethyl 1,4-dihydro-2,6-dimethyl-3,5-pyridinedicarboxylate (24.6 g, 97 mmol) in one portion. The mixture was stirred for about 4 days. The reaction mixture was extracted with 4 N aqueous HCl (5×300 mL). The organic layer was dried over Na₂SO₄, filtered and silica gel (60 g) was added. The solvents were removed under reduced pressure and the resulting solid was purified on silica gel (330 g) in two portions using a gradient of 0-30% EtOAc in heptane. The fractions containing product were combined and concentrated undser reduced pressure. The residue was dissolved in DCM (50 mL) and purified on silica gel (330 g) in three portions using a gradient of 0-26% EtOAc in DCM. The fractions containing product were combined and concentrated under reduced pressure to give (4bS,8aR)-methyl 4b-benzyl-7-oxo-4b,5,6,7,8,8a,9,10-octahydrophenanthrene-2-carboxylate (118, R²=Benzyl) (25 g, 89%) as an oil. LC/MS, method 3, Rₜ=2.70 min, no parent ion. ¹H NMR (400 MHz, DMSO-d₆) δ 7.73-7.71 (m, 1H), 7.42-7.37 (m, 1H), 7.17-7.08 (m, 3H), 6.62 (dd, J=7.7, 1.6 Hz, 2H), 6.47 (d, J=8.3 Hz, 1H), 3.82 (s, 3H), 3.33 (d, J=10.3 Hz, 1H), 3.14-2.91 (m, 3H), 2.84-2.64 (m, 2H), 2.47-2.37 (m, 1H), 2.37-2.26 (m, 1H), 2.26-2.16 (m, 1H), 2.16-2.02 (m, 1H), 2.02-1.87 (m, 1H), 1.76-1.65 (m, 1H), 1.60-1.47 (m, 1H).

### Step #2: (4bS,7R,8aR)-Methyl 4b-benzyl-7-hydroxy-7-(trifluoromethyl)-4b,5,6,7,8,8a,9,10-octahydrophenanthrene-2-carboxylate (119, R²=Benzyl, R³=Trifluoromethyl)

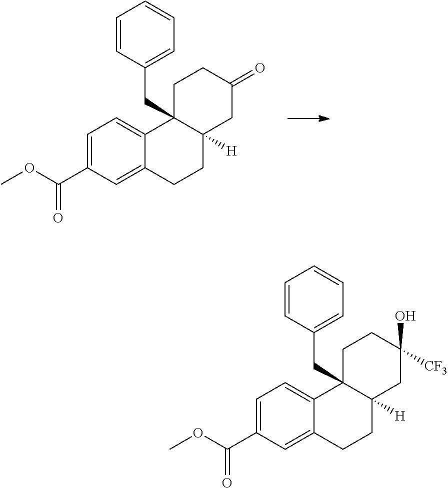

To a solution of (4bS,8aR)-methyl 4b-benzyl-7-oxo-4b,5,6,7,8,8a,9,10-octahydrophenanthrene-2-carboxylate (118, R²=Benzyl) (11.0 g, 31.6 mmol) and THF (150 mL) under N₂ at about 0° C. was added trimethyl(trifluoromethyl)silane (9.33 mL, 63.1 mmol) in THF (20 mL) in one portion. Tetrabutylammonium fluoride (1.0 M in THF) (3.16 mL, 3.16 mmol) in THF (50 mL) was added dropwise via syringe over about 90 min. The solution was stirred at about 0° C. for about 80 min. The volatiles were removed under reduced pressure. The residue was purified on silica gel (330 g) using a gradient of 0-30% EtOAc in heptane. The fractions containing product were combined and concentrated. The residue was redissolved in THF (160 mL) to give a colorless solution. The reaction was cooled to 0° C. and a solution of tetrabutylammonium fluoride(1M in THF) (27.1 mL, 27.1 mmol) in THF (80 mL) was added dropwise via dropping funnel over about 60 min and the reaction stirred for about 2 h. The reaction mixture was partitioned between EtOAc (500 mL) and saturated aqueous NaCl (100 mL). The organic layer was dried over MgSO₄, filtered and concentrated under reduced pressure. The resulting mixture was purified on silica gel (330 g) using a gradient of 0-14% EtOAc in heptane. The fractions containing product were combined and concentrated under reduced pressure to yield (4bS,7R,8aR)-methyl 4b-benzyl-7-hydroxy-7-(trifluoromethyl)-4b,5,6,7,8,8a,9,10-octahydrophenanthrene-2-carboxylate (119, R²=Benzyl, R³=Trifluoromethyl) (9.0 g, 68%) as white solid. LC/MS, method 3, Rₜ=2.64 min, MS m/z 419 (M+H)⁺. ¹H NMR (400 MHz, DMSO-d₆) δ 7.72 (d, J=1.7 Hz, 1H), 7.39 (dd, J=8.2, 1.9 Hz, 1H), 7.15-7.07 (m, 3H), 6.58-6.49 (m, 2H), 6.41 (d, J=8.3 Hz, 1H), 5.99 (s, 1H), 3.82 (s, 3H), 3.16-2.94 (m, 3H), 2.65 (d, J=13.1 Hz, 1H), 2.22-1.97 (m, 4H), 1.95-1.66 (m, 4H), 1.38-1.23 (m, 1H).

### Step #3: (4bS,7R,8aS)-Methyl 4b-benzyl-7-hydroxy-10-oxo-7-(trifluoromethyl)-4b,5,6,7,8,8a,9,10-octahydrophenanthrene-2-carboxylate (120, R²=Benzyl, R³=Trifluoromethyl)

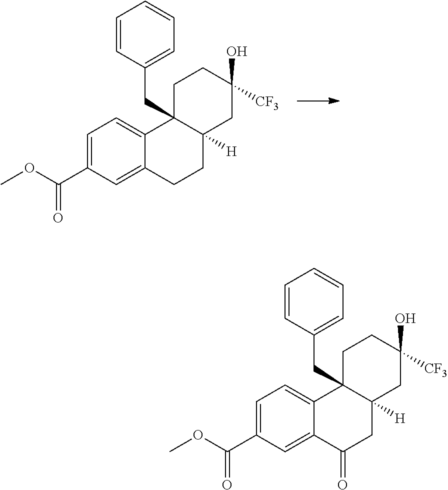

In a 500 mL round bottom flask, (4bS,7R,8aR)-methyl 4b-benzyl-7-hydroxy-7-(trifluoromethyl)-4b,5,6,7,8,8a,9,10-octahydrophenanthrene-2-carboxylate (119, R²=Benzyl, R³=Trifluoromethyl) (3.7 g, 8.84 mmol) in DCM (180 mL) and MeOH (20 mL) was cooled to about −65° C. Oxygen was bubbled through the solution (˜0.5-1 SLPM) through an L11 Ozone Gas Generator. The reaction was under ozone on and off for about 24 h. The solution was purged with oxygen for about 30 min to afford a colorless solution. Triphenylphospine (polymer bound, ˜3 mmol/g) (8.8 g, 26 mmol) was added, the cold bath was allowed to warm to rt and the reaction mixture was left to vigorously stir for about 18 h. The reaction was filtered through Celite® and washed with 10% MeOH in DCM (200 mL). The filtrate was concentrated under reduced pressure. The crude material was purified on silica gel (330 g) using a gradient of 0-11% EtOAc in DCM. The fractions containing product were combined, concentrated under reduced pressure to yield (4bS,7R,8aS)-methyl 4b-benzyl-7-hydroxy-10-oxo-7-(trifluoromethyl)-4b,5,6,7,8,8a,9,10-octahydrophenanthrene-2-carboxylate (120, R²=Benzyl, R³=Trifluoromethyl) (2.68 g, 70%) as a white solid. LC/MS, method 3, Rₜ=2.33 min, MS m/z 433 (M+H)⁺, ¹H NMR (400 MHz, DMSO-d₆) δ 8.49-8.45 (m, 1H), 7.87-7.80 (m, 1H), 7.17-7.02 (m, 3H), 6.69-6.63 (m, 1H), 6.51-6.44 (m, 2H), 6.11 (bs, 1H), 3.87 (s, 3H), 3.32-3.20 (m, 2H), 2.92-2.78 (m, 1H), 2.76-2.58 (m, 2H), 2.32-2.04 (m, 4H), 2.02-1.88 (m, 1H), 1.56-1.36 (m, 1H).

### Step 4: (4bS,7R,8aR)-Methyl 4b-benzyl-7,10-dihydroxy-7-(trifluoromethyl)-4b,5,6,7,8,8a,9,10-octahydrophenanthrene-2-carboxylate (121, R²=Benzyl, R³=Trifluoromethyl)

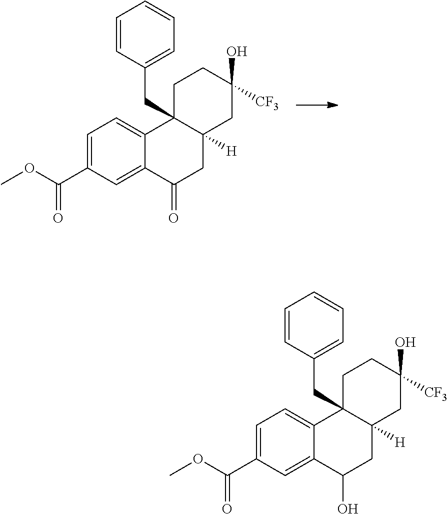

In a 200 mL round bottom flask, (4bS,7R,8aS)-methyl 4b-benzyl-7-hydroxy-10-oxo-7-(trifluoromethyl)-4b,5,6,7,8,8a,9,10-octahydrophenanthrene-2-carboxylate (120, R²=Benzyl, R³=Trifluoromethyl) (2.60 g, 6.01 mmol) was dissolved in MeOH (25 mL) and DCM (25 mL). The solution was cooled with a water bath to about 15° C. Sodium borohydride (0.227 g, 6.01 mmol) was added portionwise to the solution over about 20 min. The reaction was mixed at rt for about 2 h then quenched with 1 N aqueous HCl to about pH 5. The reaction mixture was mixed for about 1 h and then extracted with DCM (4×40 mL). The organic layer was dried over Na₂SO₄, filtered and concentrated under reduced pressure. The crude material was purified on silica gel (80 g) using a gradient of 0-40% EtOAc in DCM. The fractions containing product were combined and concentrated under reduced pressure to yield (4bS,7R,8aR)-methyl 4b-benzyl-7,10-dihydroxy-7-(trifluoromethyl)-4b,5,6,7,8,8a,9,10-octahydrophenanthrene-2-carboxylate (121, R²=Benzyl, R³=Trifluoromethyl) (2.55 g, 98%) as a white foam. LC/MS, method 3, Rₜ=2.20 min, MS m/z 493 (M+OAc)⁻. ¹H NMR (400 MHz, DMSO-d₆) δ 8.13-8.11 (m, 1H), 7.39 (dd, J=8.2, 1.6 Hz, 1H), 7.17-7.07 (m, 3H), 6.61-6.54 (m, 2H), 6.25 (d, J=8.3 Hz, 1H), 5.99 (s, 1H), 5.57-5.51 (m, 1H), 4.85-4.71 (m, 1H), 3.83 (s, 3H), 3.17 (d, J=13.1 Hz, 1H), 2.80 (d, J=13.3 Hz, 1H), 2.24-2.08 (m, 2H), 2.06-1.80 (m, 5H), 1.36-1.22 (m, 1H).

### Step #5: (4bS,7R,8aS)-Methyl 4b-benzyl-7-hydroxy-7-(trifluoromethyl)-4b,5,6,7,8,8a-hexahydrophenanthrene-2-carboxylate (122, R²=Benzyl, R³=Trifluoromethyl)

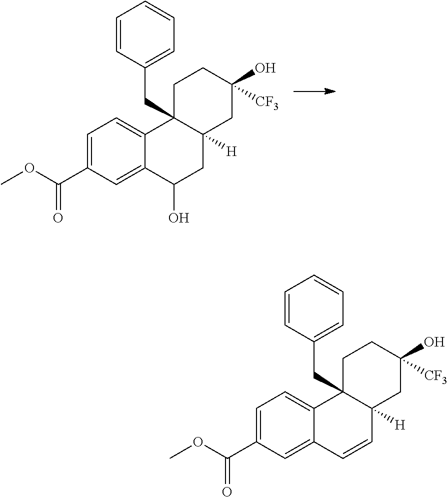

In a 50 mL round bottom flask, (4bS,7R,8aR)-methyl 4b-benzyl-7,10-dihydroxy-7-(trifluoromethyl)-4b,5,6,7,8,8a,9,10-octahydrophenanthrene-2-carboxylate (121, R²=Benzyl, R³=Trifluoromethyl) (0.250 g, 0.575 mmol) was dissolved in toluene (20 mL). 4 Å molecular sieves (0.6 g) and 4-methylbenzenesulfonic acid hydrate (11 mg, 0.058 mmol) were added and the reaction was mixed at rt for about 10 min and then at about 60° C. for about 4 h. The reaction mixture was filtered into saturated aqueous NaHCO₃ (30 mL), rinsing with EtOAc (100 mL). The layers were separated and the organics were washed with saturated aqueous NaCl (20 mL). The aqueous phases were back extracted with EtOAc (30 mL). The combined organics were dried over MgSO₄, filtered, and concentrated under reduced pressure. The crude material was purified on silica gel (80 g) using a gradient of 20-60% EtOAc in DCM. The fractions containing product were combined and concentrated to yield (4bS,7R,8aS)-methyl 4b-benzyl-7-hydroxy-7-(trifluoromethyl)-4b,5,6,7,8,8a-hexahydrophenanthrene-2-carboxylate (122, R²=Benzyl, R³=Trifluoromethyl) (0.127 g, 53%) as a white solid. LC/MS, method 3, Rₜ=2.59 min, MS m/z 475 (M+OAc)⁻. ¹H NMR (600 MHz, DMSO-d₆) δ 7.80-7.77 (m, 1H), 7.51-7.47 (m, 1H), 7.10-7.00 (m, 3H), 6.79 (dd, J=9.4, 2.9 Hz, 1H), 6.46 (d, J=8.1 Hz, 1H), 6.36 (d, J=7.2 Hz, 2H), 6.08 (s, 1H), 5.92-5.88 (m, 1H), 3.84 (s, 3H), 2.95 (d, J=13.3 Hz, 1H), 2.84 (d, J=13.2 Hz, 1H), 2.69-2.63 (m, 1H), 2.27-2.20 (m, 2H), 2.20-2.11 (m, 1H), 2.111-2.01 (m, 2H), 1.54-1.45 (m, 1H).

### Step #6: (4bS,7R,8aS)-4b-Benzyl-7-hydroxy-N-(2-methylpyridin-3-yl)-7-(trifluoromethyl)-4b,5,6,7,8,8a-hexahydrophenanthrene-2-carboxamide (123, R²=Benzyl, R³=Trifluoromethyl, R⁶=2-Methylpyridin-3-yl)

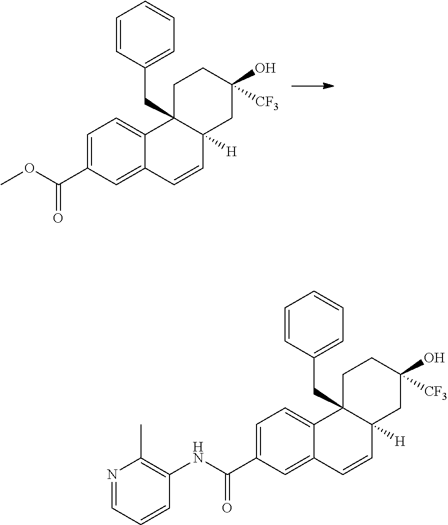

Toluene (3.0 mL) and THF (3.0 mL) were added to (4bS,7R,8aS)-methyl 4b-benzyl-7-hydroxy-7-(trifluoromethyl)-4b,5,6,7,8,8a-hexahydrophenanthrene-2-carboxylate (122, R²=Benzyl, R³=Trifluoromethyl) (0.127 g, 0.305 mmol) and the solution was cooled to about 0° C., then 2-methylpyridin-3-amine (0.040 g, 0.366 mmol) was added in one portion. LiHMDS (1 M solution in THF, 0.92 mL, 0.92 mmol) was added dropwise and the reaction was stirred for about 30 min at about 0° C. The ice bath was removed and the reaction mixture was stirred at rt for about 60 min. Saturated aqueous NaHCO₃ (10 mL) was added and the biphasic solution was extracted with EtOAc (2×25 mL). The combined organic layers were dried over Na₂SO₄, filtered, and concentrated under reduced pressure. The residue was purified on silica gel (12 g) using a gradient of 0-20% EtOAc in DCM. The fractions containing product were combined and concentrated under reduced pressure to yield (4bS,7R,8aS)-4b-benzyl-7-hydroxy-N-(2-methylpyridin-3-yl)-7-(trifluoromethyl)-4b,5,6,7,8,8a-hexahydrophenanthrene-2-carboxamide (123, R²=Benzyl, R³=Trifluoromethyl, R⁶=2-Methylpyridin-3-yl) (124 mg, 83%) as a solid. LC/MS, method 3, Rₜ=2.15 min, MS m/z 493 (M+H)⁺. ¹H NMR (400 MHz, DMSO-d₆) δ 9.97 (s, 1H), 8.36-8.32 (m, 1H), 7.85-7.79 (m, 1H), 7.78-7.72 (m, 1H), 7.59-7.51 (m, 1H), 7.32-7.23 (m, 1H), 7.15-7.01 (m, 3H), 6.84-6.74 (m, 1H), 6.53-6.46 (m, 1H), 6.46-6.37 (m, 2H), 6.11 (s, 1H), 5.96-5.88 (m, 1H), 2.98 (d, J=13.2 Hz, 1H), 2.88 (d, J=13.2 Hz 1H), 2.73-2.63 (m, 1H), 2.44 (s, 3H), 2.31-2.19 (m, 2H), 2.20-1.98 (m, 3H), 1.62-1.42 (m, 1H).

### Step #7: 4-((1S,2R,4R)-1-Benzyl-4-hydroxy-2-(hydroxymethyl)-4-(trifluoromethyl)cyclohexyl)-3-(hydroxymethyl)-N-(2-methylpyridin-3-yl)benzamide (124, R²=Benzyl, R³=Trifluoromethyl, R⁶=2-Methylpyridin-3-yl)

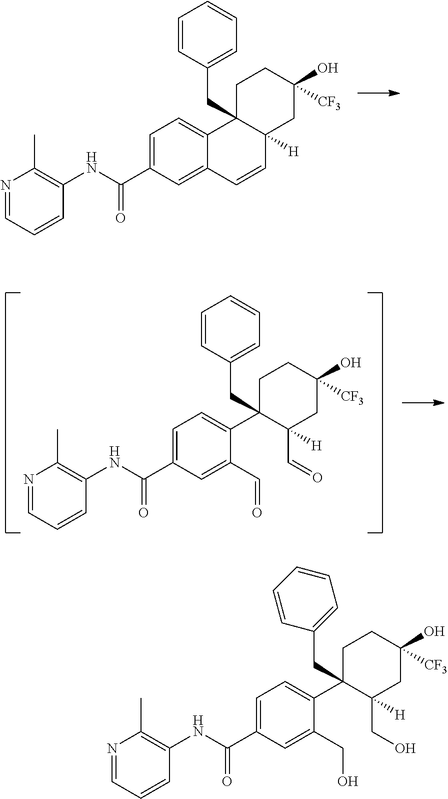

DCM (9 mL) and MeOH (1 mL) were added to (4bS,7R,8aS)-4b-benzyl-7-hydroxy-N-(2-methylpyridin-3-yl)-7-(trifluoromethyl)-4b,5,6,7,8,8 a-hexahydrophenanthrene-2-carboxamide (123, R²=Benzyl, R³=Trifluoromethyl, R⁶=2-Methylpyridin-3-yl) (124 mg, 0.252 mmol) and the mixture was cooled to about −78° C. The mixture was treated with ozone for about 5 min to obtain a blue solution. The reaction was sealed and mixed for about 30 min and then the reaction was purged with O₂ for about 30 min. Polymer supported triphenylphosphine (˜3 mmol/g, 0.50 g) was added and the reaction was mixed at about 0° C. for about 30 min, then at rt for about 1 h. The mixture was filtered and washed with 50% MeOH in DCM (10 mL). The filtrate was treated with sodium borohydride (29 mg, 0.76 mmol) and the reaction was mixed at rt for about 1 h. The volatiles were removed under reduced pressure and the residue was distributed between water (10 mL) and DCM (10 mL) and then treated with 1 N aqueous HCl (2 mL). The biphasic mixture was stirred for about 2 h, diluted with saturated aqueous NaCl (10 mL) and extracted with DCM (4×10 mL). The combined organic layers were washed with saturated aqueous NaCl (10 mL), dried over Na₂SO₄, filtered, and concentrated under reduced pressure. The residue was purified on silica gel (12 g) using a gradient of 2-15% MeOH in DCM. The fractions containing product were combined and concentrated under reduced pressure to yield 44(1S,2R,4R)-1-benzyl-4-hydroxy-2-(hydroxymethyl)-4-(trifluoromethyl)cyclohexyl)-3-(hydroxymethyl)-N-(2-methylpyridin-3-Abenzamide (124, R²=Benzyl, R³=Trifluoromethyl, R⁶=2-Methylpyridin-3-yl) (44 mg, 32%) as a solid. LC/MS, method 2, Rₜ=1.72 min, MS m/z 529 (M+H)⁺. ¹H NMR (600 MHz, DMSO-d₆) δ 9.97 (s, 1H), 8.33 (d, J=4.7 Hz, 1H), 8.23 (s, 1H), 7.74 (d, J=7.8 Hz, 1H), 7.56 (d, J=8.1 Hz, 1H), 7.27 (dd, J=7.8, 4.7 Hz, 1H), 7.06-6.97 (m, 3H), 6.85-6.77 (m, 1H), 6.60-6.56 (m, 2H), 5.77-5.73 (m, 1H), 5.43-5.37 (m, 1H), 5.04-4.78 (m, 3H), 4.12-4.05 (m, 1H), 4.01-3.94 (m, 1H), 3.41-3.34 (m, 1H), 2.96-2.87 (m, 2H), 2.44 (s, 3H), 2.13-2.00 (m 2H), 1.80-1.59 (m, 3H), 1.28-1.17 (m, 1H).

### Step #8: (7aR,9R,11aS)-11a-Benzyl-9-hydroxy-N-(2-methylpyridin-3-yl)-5-oxo-9-(trifluoromethyl)-5,7,7a,8,9,10,11,11a-octahydrodibenzo[c,e]oxepine-3-carboxamide (125, R²=Benzyl, R³=Trifluoromethyl, R⁶=2-Methylpyridin-3-yl)

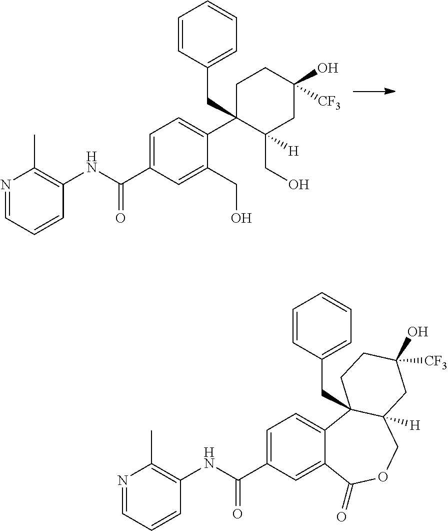

DCM (4 mL) and THF (0.2 mL) were added to 4-((1S,2R,4R)-1-benzyl-4-hydroxy-2-(hydroxymethyl)-4-(trifluoromethyl)cyclohexyl)-3-(hydroxymethyl)-N-(2-methylpyridin-3-yl)benzamide (124, R²=Benzyl, R³=Trifluoromethyl, R⁶=2-Methylpyridin-3-yl (25 mg, 0.047 mmol). Manganese dioxide (82 mg, 0.95 mmol) was added and the reaction was mixed at rt for about 72 h. The reaction mixture was filtered through Celite® (500 mg), rinsing with 10% MeOH in DCM (5.0 mL). The filtrate was concentrated under reduced pressure and the residue was purified on silica gel (4 g) using a gradient of 10-90% EtOAc in DCM. The fractions containing product were combined and concentrated to yield (7aR,9R,11aS)-11a-benzyl-9-hydroxy-N-(2-methylpyridin-3-yl)-5-oxo-9-(trifluoromethyl)-5,7,7a,8,9,10,11,11a-octahydrodibenzo[c,e]oxepine-3-carboxamide (125, R²=Benzyl, R³=Trifluoromethyl, R⁶=2-Methylpyridin-3-yl) (18 mg, 71%) as a solid. LC/MS, method 2, Rₜ=1.93 min, MS m/z 525 (M+H)⁺. ¹H NMR (400 MHz, DMSO-d₆) δ 10.25 (s, 1H), 8.38-8.33 (m, 2H), 8.10-8.02 (m, 1H), 7.78-7.71 (m, 1H), 7.33-7.24 (m, 1H), 7.23-7.15 (m, 1H), 7.11-7.00 (m, 3H), 6.70-6.59 (m, 2H), 6.21 (s, 1H), 4.42 (dd, J=13.5, 7.0 Hz, 1H), 4.24-4.15 (m, 1H), 3.52 (d, J=14.4 Hz, 1H), 3.10 (d, J=14.7 Hz, 1H), 2.48-2.38 (m, 1H), 2.44 (s, 3H), 2.35-2.11 (m, 4H), 1.96-1.76 (m, 2H).

### Example #116

### (7aR,9R,11aS)-11a-Benzyl-9-hydroxy-N-(2-methylpyridin-3-yl)-9-(trifluoromethyl)-5,7,7a,8,9,10,11,11a-octahydrodibenzo[c,e]oxepine-3-carboxamide (126, R²=Benzyl, R³=Trifluoromethyl, R⁶=2-Methylpyridin-3-yl)

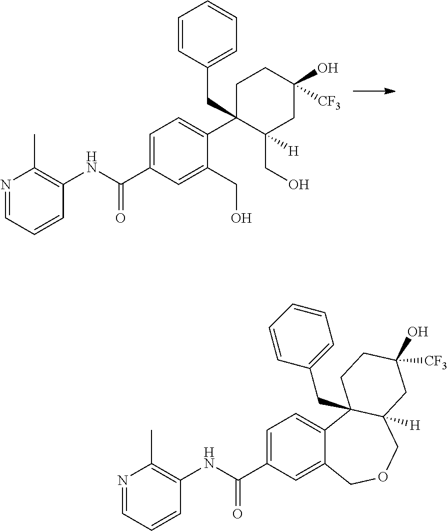

Dichloroethane (1 mL) and pyridine (0.2 mL) were added to 4-((1S,2R,4R)-1-benzyl-4-hydroxy-2-(hydroxymethyl)-4-(trifluoromethyl)cyclohexyl)-3-(hydroxymethyl)-N-(2-methylpyridin-3-yl)benzamide (124, R²=Benzyl, R³=Trifluoromethyl, R⁶=2-Methylpyridin-3-yl) (28 mg, 0.050 mmol) at rt. A solution of p-toluenesulfonyl chloride (19 mg, 0.101 mmol) in dichloroethane (1 mL) was added dropwise via syringe and the reaction was stirred at rt for about 2 min. TEA (0.05 mL, 0.35 mmol) was added and the reaction was stirred at rt for about 90 min and then at about 50° C. for about 30 min. p-Toluenesulfonyl chloride (14 mg) in dichloroethane (1.0 mL) was added and the reaction was stirred at about 50° C. for about 30 min. p-Toluenesulfonyl chloride (8 mg, 0.042 mmol) dichloroethane (0.5 mL) was added and the reaction was stirred at about 60° C. for about 4 h. p-Toluenesulfonyl chloride (17 mg, 0.089 mmol) in dichloroethane (0.5 mL) and TEA (0.10 mL, 0.72 mmol) were added and the reaction was stirred at about 60° C. for about 3 h. The solvents were removed under reduced pressure and the residue was dissolved in DCM (20 mL) and washed with NaHCO₃ (2×20 mL). The organic layer was dried over Na₂SO₄, filtered and concentrated under reduced pressure. The residue was purified on silica gel (4 g) using a gradient of 0-100% EtOAc in DCM. The fractions containing product were combined, concentrated under reduced pressure and lyophilized to yield (7aR,9R,11aS)-11a-benzyl-9-hydroxy-N-(2-methylpyridin-3-yl)-9-(trifluoromethyl)-5,7,7a,8,9,10,11,11a-octahydrodibenzo[c,e]oxepine-3-carboxamide (126, R²=Benzyl, R³=Trifluoromethyl, R⁶=2-Methylpyridin-3-yl) (7 mg, 25%) as a white solid. LC/MS, method 2, Rₜ=2.15 min, MS m/z 511 (M+H)⁺. ¹H NMR (600 MHz, DMSO-d₆) δ 10.12 (s, 1H), 8.43-8.37 (m, 1H), 8.29-8.26 (m, 1H), 7.94-7.86 (m, 1H), 7.69-7.66 (m, 1H), 7.43-7.34 (m, 1H), 7.09-7.00 (m, 4H), 6.72-6.66 (m, 2H), 5.45 (bs, 1H), 4.94 (d, J=13.1 Hz, 1H), 4.81 (d, J=13.1 Hz, 1H), 4.55-4.51 (m, 1H), 4.04 (dd, J=9.2, 4.8 Hz, 1H), 3.26 (d, J=13.2 Hz, 1H), 2.93 (d, J=13.2 Hz, 1H), 2.51-2.48 (m, 1H), 2.49 (s, 3H), 2.13-2.04 (m, 1H), 1.96-1.87 (m, 2H), 1.76-1.69 (m, 1H), 1.62-1.51 (m, 1H), 1.33-1.26 (m, 1H).

### Example #117

### (3R,4aR,11bS)-11b-Benzyl-3-hydroxy-6-methyl-N-(2-methylpyridin-3-yl)-3-(trifluoromethyl)-2,3,4,4a,5,6,7,11b-octahydro-1H-dibenzo[c,e]azepine-9-carboxamide (127, R²=Benzyl, R³=Trifluoromethyl, R⁶=2-Methylpyridin-3-yl)

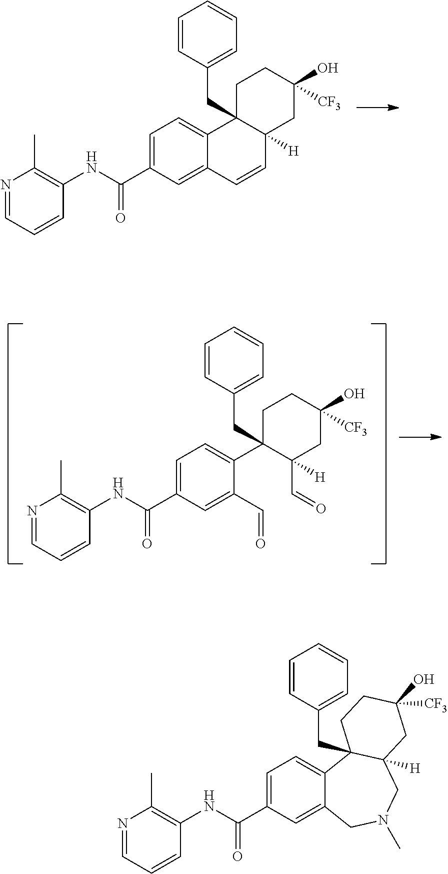

DCM (9 mL) and MeOH (1 mL) were added to (4bS,7R,8aS)-4b-benzyl-7-hydroxy-N-(2-methylpyridin-3-yl)-7-(trifluoromethyl)-4b,5,6,7,8,8a-hexahydrophenanthrene-2-carboxamide (123, R²=Benzyl, R³=Trifluoromethyl, R⁶=2-Methylpyridin-3-yl) (0.11 g, 0.22 mmol) at rt. The mixture was cooled to about −78° C. The reaction was treated with a stream of Ozone for about 15 min and then the reaction was sealed and mixed for about 30 min. The reaction was purged with O₂ for about 20 min. Polymer supported triphenylphosphine (˜3 mmol/g, 0.25 g) was added and mixture was stirred at rt for about 1 h. The reaction mixture was filtered through Celite® and washed with 50% MeOH in DCM (10 mL). The filtrate was concentrated under reduced pressure to a white solid and diluted with MeCN (6 mL) to give a white suspension. THF (0.5 mL) and 2 M methylamine in THF (0.34 mL, 0.69 mmol) was added and the reaction was stirred for about 10 min. Sodium cyanoborohydride (115 mg, 1.83 mmol) was added and the mixture was stirred for about 18 h. Sodium cyanoborohydride (60 mg, 0.955 mmol) was added to the reaction and the reaction mixture was stirred for about 18 h. Saturated aqueous NaHCO₃ (10 mL) was added and the mixture was extracted with EtOAc (3×20 mL). The combined organics were washed with saturated aqueous NaCl, dried over MgSO₄, filtered and concentrated under reduced pressure. The residue was purified on silica gel (12 g) using a gradient of 0-10% MeOH in DCM then 10% MeOH in DCM with 1% 7N NH₃ in MeOH. The fractions containing product were combined and concentrated under reduced pressure. The residue was purified by chiral column using step-wise purification: Step 1: The gradient was 5-28% A in 13 min (20 mL/min flow rate). Mobile phase A was EtOH (200 proof), mobile phase B was HPLC grade heptane with 0.12% DEA added. The chromatography used a Viridis 2-ethylpyridine mm column (5 μm particles) from Waters Corporation. Product was the second eluting component (r.t. 11.9 min) in the 1st step of purification. Step 2: The gradient was 5-30% A in 14 min (20 mL/min flow rate). Mobile phase A was EtOH (200 proof), mobile phase B was HPLC grade heptane with 0.12% DEA added. The chromatography used a Daicel IB, 20×250 mm column (5 μm particles). The product was again the second eluting component (r.t. 12.9 min) The fractions containing product were combined, concentrated and lyophilized to give (3R,4aR,11bS)-11b-benzyl-3-hydroxy-6-methyl-N-(2-methylpyridin-3-yl)-3-(trifluoromethyl)-2,3,4,4a,5,6,7,11b-octahydro-1H-dibenzo[c,e]azepine-9-carboxamide (127, R²=Benzyl, R³=Trifluoromethyl, R⁶=2-Methylpyridin-3-y0 (2.5 mg, 2%). LC/MS, method 2, Rₜ=1.02 min, MS m/z 524 (M+H)⁺. ¹H NMR (600 MHz, DMSO-d₆) δ 9.95 (s, 1H), 8.33 (d, J=3.9 Hz, 1H), 7.80 (s, 1H), 7.73 (d, J=7.8 Hz, 1H), 7.59 (d, J=8.0 Hz, 1H), 7.27 (dd, J=8.0, 4.7 Hz, 1H), 7.12-7.04 (m, 3H), 6.86 (d, J=8.3 Hz, 1H), 6.60-6.55 (m, 2H), 5.99 (s, 1H), 4.39 (d, J=15.0 Hz, 1H), 3.87 (d, J=15.2 Hz, 1H), 3.54 (d, J=13.7 Hz, 1H), 3.11 (dd, J=12.0, 12.0 Hz, 1H), 3.00 (d, J=13.7 Hz, 1H), 2.64-2.59 (m, 1H), 2.43 (s, 3H), 2.30 (s, 3H), 2.25-2.16 (m, 1H), 2.06-1.88 (m, 3H), 1.85-1.79 (m, 1H), 1.73-1.62 (m, 2H).

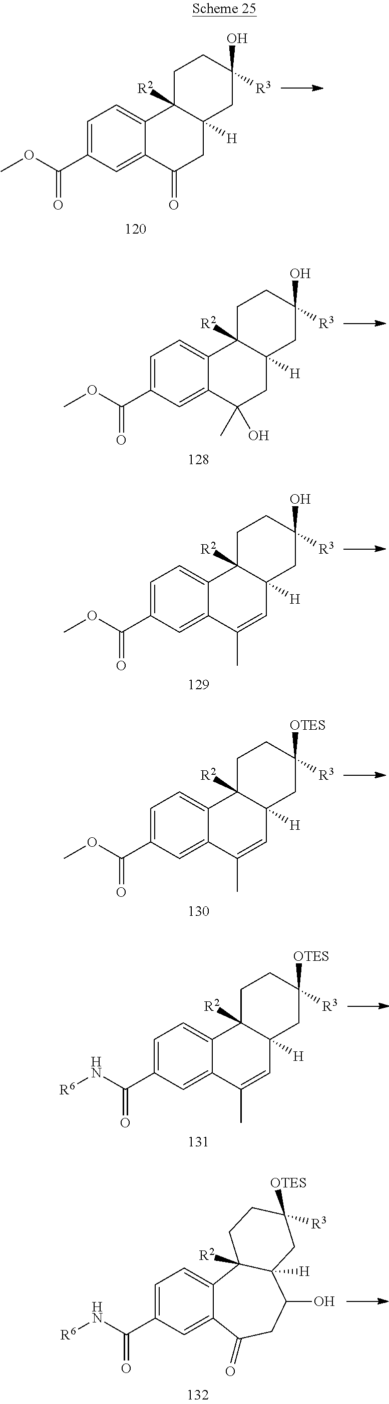

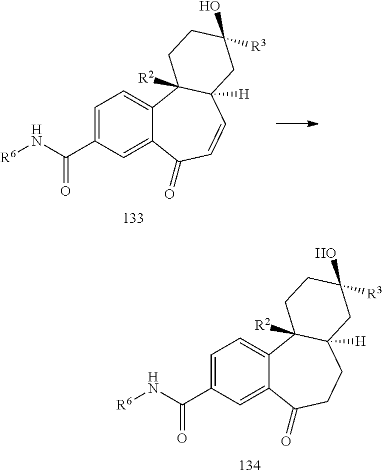

### Example #118

### (7aR,9R,11aS)-11a-Benzyl-9-hydroxy-5-oxo-9-trifluoromethyl-6,7,7a,8,9,10,11,11a-octahydro-5H-dibenzo[a,c]cycloheptene-3-carboxylic acid (2-methyl-pyridin-3-yl)-amide (134, R²=Benzyl, R³=Trifluoromethyl, R⁶=2-Methylpyridin-3-yl)

### Step #1: (4bS,7R,8aR)-Methyl 4b-benzyl-7,10-dihydroxy-10-methyl-7-(trifluoromethyl)-4b,5,6,7,8,8a,9,10-octahydrophenanthrene-2-carboxylate (128, R²=Benzyl, R³=Trifluoromethyl)

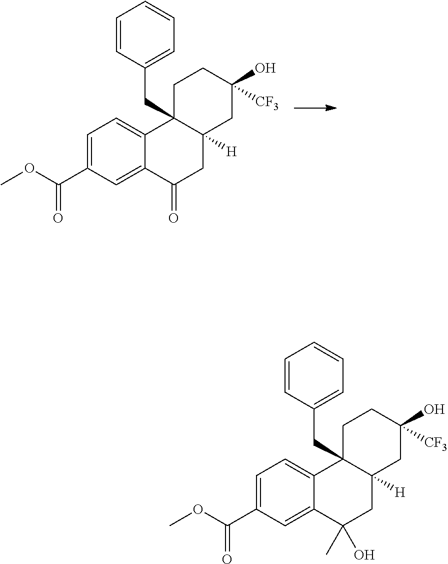

Methylmagnesium bromide (3.0 M solution in Et₂O, 1.20 mL, 3.60 mmol) was added dropwise to a solution of (4bS,7R,8aS)-methyl 4b-benzyl-7-hydroxy-10-oxo-7-(trifluoromethyl)-4b,5,6,7,8,8a,9,10-octahydrophenanthrene-2-carboxylate (120, R²=Benzyl, R³=Trifluoromethyl) (0.247 g, 0.571 mmol) and THF (10 mL) under a nitrogen atmosphere at about −78° C. over about 15 min. The reaction vessel was allowed to warm to between about −20 to −25° C. over about 15 min and then maintained within that temperature range for about 45 min. The reaction mixture was cooled to about −40° C. and MeOH (0.2 mL) was added dropwise. The reaction vessel was removed from the cold bath and saturated aqueous NH₄Cl (25 mL) and EtOAc (25 mL) were added. Water was added to dissolve the salts. The layers were separated and the aqueous phase was extracted with additional EtOAc (25 mL). The combined organics were dried over Na₂SO₄, filtered, and concentrated under reduced pressure. The residue was purified on silica gel (25 g) using a gradient of 0-75% EtOAc in heptane. The fractions containing product were combined and concentrated under reduced pressure to afford (4bS,7R,8aR)-methyl 4b-benzyl-7,10-dihydroxy-10-methyl-7-(trifluoromethyl)-4b,5,6,7,8,8a,9,10-octahydrophenanthrene-2-carboxylate (128, R²=Benzyl, R³=Trifluoromethyl) (0.143 g, 56%) as an ivory solid. LC/MS, method 3, Major Isomer: Rₜ=2.31 min, MS m/z 431 (M−OH)⁺, Minor Isomer: Rₜ=2.28 min, MS m/z 431 (M−OH)⁺, Major Isomer: ¹H NMR (400 MHz, DMSO-d₆) δ 8.20 (d, J=1.9 Hz, 1H), 7.31 (dd, J=8.2, 1.9 Hz, 1H), 7.17-7.07 (m, 3H), 6.58-6.53 (m, 2H), 6.13 (d, J=8.3 Hz, 1H), 6.01 (s, 1H), 5.31 (s, 1H), 3.83 (s, 3H), 3.15 (d, J=13.1 Hz, 1H), 2.74 (d, J=12.8 Hz, 1H), 2.34-1.81 (m, 7H), 1.72-1.65 (m, 1H), 1.36 (s, 3H), 1.33-1.21 (m, 1H).

### Step #2: (4bS,7R,8aS)-Methyl 4b-benzyl-7-hydroxy-10-methyl-7-(trifluoromethyl)-4b,5,6,7,8,8a-hexahydrophenanthrene-2-carboxylate (129, R²=Benzyl, R³=Trifluoromethyl)

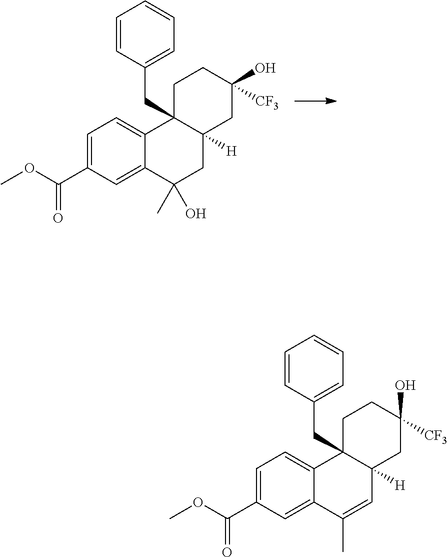

4 Å Molecular sieves (4.0 g) were added to a solution of (4bS,7R,8aR)-methyl 4b-benzyl-7,10-dihydroxy-10-methyl-7-(trifluoromethyl)-4b,5,6,7,8,8a,9,10-octahydrophenanthrene-2-carboxylate (128, R²=Benzyl, R³=Trifluoromethyl) (0.625 g, 1.39 mmol) and toluene (30 mL) under a nitrogen atmosphere. p-Toluenesulfonic acid monohydrate (0.050 g, 0.26 mmol) was added in one portion. The mixture was warmed to about 60° C. for about 45 min. The mixture was allowed to cool to rt and then filtered into saturated aqueous NaHCO₃ (25 mL) rinsing with EtOAc (25 mL). The layers were separated and the organics were washed with saturated aqueous NaCl (25 mL). The aqueous layers were extracted with EtOAc (25 mL). The combined organics were dried over Na₂SO₄, filtered, and concentrated under reduced pressure. The residue was purified on silica gel (40 g) using a gradient of 0-50% EtOAc in heptane. The fractions containing product were combined and concentrated under reduced pressure to afford (4bS,7R,8aS)-methyl 4b-benzyl-7-hydroxy-10-methyl-7-(trifluoromethyl)-4b,5,6,7,8,8a-hexahydrophenanthrene-2-carboxylate (129, R²=Benzyl, R³=Trifluoromethyl) (0.599 g, quant.) as an ivory solid/foam. LC/MS, method 3, Rₜ=2.71 min, no parent mass, ¹H NMR (400 MHz, DMSO-d₆) δ 7.86 (d, J=1.8 Hz, 1H), 7.53 (dd, J=8.0, 1.8 Hz, 1H), 7.10-6.97 (m, 3H), 6.49 (d, J=8.1 Hz, 1H), 6.38-6.32 (m, 2H), 6.08 (s, 1H), 5.72-5.67 (m, 1H), 3.85 (s, 3H), 2.93 (d, J=13.2 Hz, 1H), 2.81 (d, J=13.0 Hz, 1H), 2.65-2.56 (m, 1H), 2.27-1.97 (m, 8H), 1.55-1.42 (m, 1H).

### Step #3: (4bS,7R,8aS)-Methyl 4b-benzyl-10-methyl-7-(triethylsilyloxy)-7-(trifluoromethyl)-4b,5,6,7,8,8a-hexahydrophenanthrene-2-carboxylate (130, R²=Benzyl, R³=Trifluoromethyl)

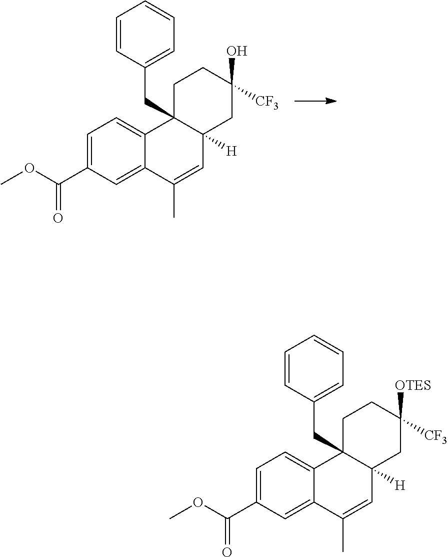

LiHMDS (1.0 M solution in THF, 1.20 mL, 1.20 mmol) was added dropwise to a solution of (4bS,7R,8aS)-methyl 4b-benzyl-7-hydroxy-10-methyl-7-(trifluoromethy 1)-4b,5,6,7,8,8 a-hexahydrophenanthrene-2-carboxylate (129, R²=Benzyl, R³=Trifluoromethyl) (0.359 g, 0.835 mmol) and THF (8 mL) under a nitrogen atmosphere at about 0° C. After about 10 min, chlorotriethylsilane (0.240 mL, 1.43 mmol) was added in one portion. After about 15 min, the ice bath was removed. After about 90 min, the solution was cooled to about 0° C. LiHMDS (1.0 M solution in THF, 0.600 mL, 0.600 mmol) was added. After about 5 min, chlorotriethylsilane (0.120 mL, 0.715 mmol) was added. After about 5 min, the ice bath was removed. After about 1 h, the solution was poured into saturated aqueous NaHCO₃ (50 mL) and then extracted with EtOAc (2×20 mL). The combined organics were dried over Na₂SO₄, filtered, and concentrated under reduced pressure. The residue was purified on silica gel (40 g) using a gradient of 0-10% EtOAc in heptane. The fractions containing product were combined and concentrated under reduced pressure to afford (4bS,7R,8aS)-methyl 4b-benzyl-10-methyl-7-(triethylsilyloxy)-7-(trifluoromethyl)-4b,5,6,7,8,8a-hexahydrophenanthrene-2-carboxylate (130, R²=Benzyl, R³=Trifluoromethyl) (0.423 g, 90%) as a pale yellow-white sticky foam/film. LC/MS, method 4, Rₜ=3.25 min, MS m/z 546 (M+H)⁺. ¹H NMR (400 MHz, DMSO-d₆) δ 7.85 (d, J=1.8 Hz, 1H), 7.52 (dd, J=8.0, 1.8 Hz, 1H), 7.10-7.00 (m, 3H), 6.47 (d, J=8.1 Hz, 1H), 6.36-6.30 (m, 2H), 5.75-5.71 (m, 1H), 3.84 (s, 3H), 2.88 (d, J=13.0 Hz, 1H), 2.78 (d, J=13.1 Hz, 1H), 2.69-2.57 (m, 1H), 2.41-2.24 (m, 2H), 2.18-1.99 (m, 6H), 1.59-1.46 (m, 1H), 0.98 (t, J=7.8 Hz, 9H), 0.71 (q, J=7.8 Hz, 6H).

### Step #4: (4bS,7R,8aS)-4b-Benzyl-10-methyl-N-(2-methylpyridin-3-yl)-7-(triethylsilyloxy)-7-(trifluoromethyl)-4b,5,6,7,8,8a-hexahydrophenanthrene-2-carboxamide (131, R²=Benzyl, R³=Trifluoromethyl, R⁶=2-Methylpyridin-3-yl)

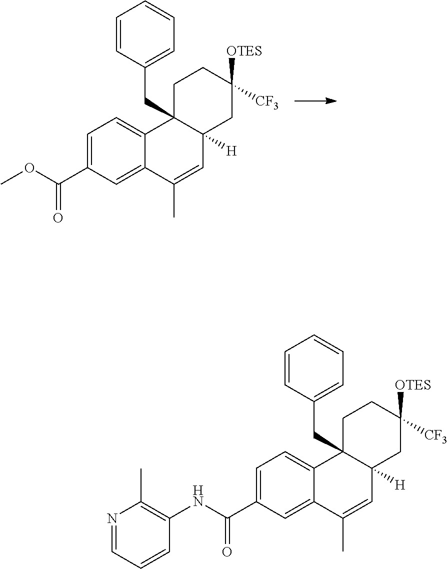

LiHMDS (1.0 M solution in THF, 2.30 mL, 2.30 mmol) was added dropwise to a solution of (4bS,7R,8aS)-methyl 4b-benzyl-10-methyl-7-(triethylsilyloxy)-7-(trifluoromethyl)-4b,5,6,7,8,8a-hexahydrophenanthrene-2-carboxylate (130, R²=Benzyl, R³=Trifluoromethyl) (0.364 g, 0.668 mmol), 3-amino-2-methylpyridine (0.108 g, 1.00 mmol), and toluene (6.50 mL) under a nitrogen atmosphere at about 0° C. After about 1 h, saturated aqueous NaHCO₃ (5 mL) and water (5 mL) were added. The solution was extracted with EtOAc (2×10 mL). The combined organics were washed with saturated aqueous NaCl (5 mL), dried over Na₂SO₄, filtered, and concentrated under reduced pressure. The residue was purified on silica gel (25 g) using a gradient of 0-50% EtOAc in DCM. The fractions containing product were combined and concentrated under reduced pressure to afford (4bS,7R,8aS)-4b-benzyl-10-methyl-N-(2-methylpyridin-3-yl)-7-(triethylsilyloxy)-7-(trifluoromethyl)-4b,5,6,7,8,8a-hexahydrophenanthrene-2-carboxamide (131, R²=Benzyl, R³=Trifluoromethyl, R⁶=2-Methylpyridin-3-yl) (0.383 g, 92%) of a pale yellow film/glass. LC/MS, method 3, Rₜ=3.44 min, MS m/z 622 (M+H)⁺. ¹H NMR (400 MHz, DMSO-d₆) δ 10.01 (s, 1H), 8.35-8.31 (m, 1H), 7.93 (s, 1H), 7.74 (d, J=7.6 Hz, 1H), 7.58 (d, J=6.3 Hz, 1H), 7.27 (dd, J=7.8, 4.9 Hz, 1H), 7.11-7.03 (m, 3H), 6.49 (d, J=8.2 Hz, 1H), 6.43-6.37 (m, 2H), 5.73 (s, 1H), 2.91 (d, J=13.1 Hz, 1H), 2.81 (d, J=13.1 Hz, 1H), 2.69-2.59 (m, 1H), 2.42 (s, 3H), 2.40-2.26 (m, 2H), 2.19 (s, 3H), 2.15-2.01 (m, 3H), 1.60-1.46 (m, 1H), 0.99 (t, J=7.9 Hz, 9H), 0.72 (q, J=7.8 Hz, 6H).

### Step #5: (7aR,9R,11aS)-11a-Benzyl-7-hydroxy-5-oxo-9-triethylsilanyloxy-9-trifluoromethyl-6,7,7a,8,9,10,11,11a-octahydro-5H-dibenzo[a,c]cycloheptene-3-carboxylic acid (2-methyl-pyridin-3-yl)-amide (132, R²=Benzyl, R³=Trifluoromethyl, R⁶=2-Methylpyridin-3-yl)

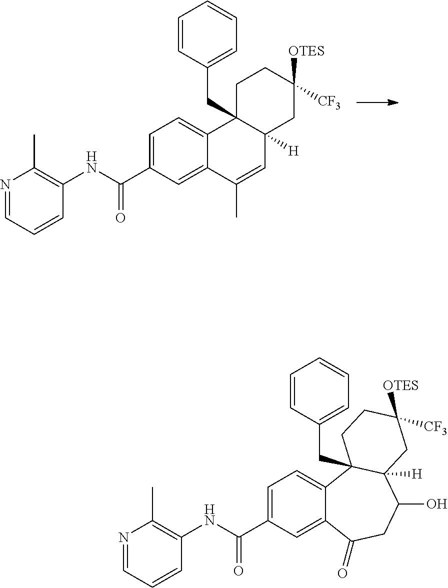

A solution of (4bS,7R,8aS)-4b-benzyl-10-methyl-N-(2-methylpyridin-3-yl)-7-(triethylsilyloxy)-7-(trifluoromethyl)-4b,5,6,7,8,8a-hexahydrophenanthrene-2-carboxamide (131, R²=Benzyl, R³=Trifluoromethyl, R⁶=2-Methylpyridin-3-yl) (0.422 g, 0.680 mmol), DCM (11.3 mL) and MeOH (2.30 mL) was purged with O₂ at about −78° C. for about 5 min. Oxygen was bubbled through the solution (0.5-1.0 SLPM, Reactor Pressure=5-6 psi) through an L11 Ozone Gas Generator. After about 6 min, the solution turned a faint blue. The solution was purged with O₂ for about 15 min. Polymer-bound triphenylphosphine (˜3 mmol/g, 1.0 g, 3.0 mmol) was added. The mixture was allowed to warm to rt over about 30 min. After about 5 h, the mixture was filtered rinsing with 50% MeOH in DCM (40 mL). 0.5 M aqueous NaOH (1.50 mL, 0.750 mmol) was added to the organics. After stirring for about 45 min, saturated aqueous NaHCO₃ (10 mL) and water (10 mL) were added. The layers were separated and the aqueous phase was extracted with DCM (2×20 mL). The combined organics were concentrated under reduced pressure. The residue was dissolved in water (40 mL) and DCM (40 mL). The layers were separated and the aqueous phase was extracted with DCM (2×20 mL). The organics were washed with saturated aqueous NaCl (20 mL), dried over Na₂SO₄, filtered, and concentrated under reduced pressure. The residue was purified on silica gel (40 g) using a gradient of 20-75% EtOAc in DCM. The fractions containing product were combined and concentrated under reduced pressure to afford about a 55:45 ratio of alcohol diastereomers of (7aR,9R,11aS)-11a-benzyl-7-hydroxy-5-oxo-9-triethylsilanyloxy-9-trifluoromethyl-6,7,7a,8,9,10,11,11a-octahydro-5H-dibenzo[a,c]cycloheptene-3-carboxylic acid (2-methyl-pyridin-3-yl)-amide (132, R²=Benzyl, R³=Trifluoromethyl, R⁶=2-Methylpyridin-3-yl) (0.279 g, 63%) as an ivory solid. Major Isomer:LC/MS, method 3, Rₜ=3.00 min, MS m/z 654 (M+H)⁺. Minor Isomer: LC/MS, method 3, Rₜ=2.95 min, MS m/z 654 (M+H)⁺. Major isomer: ¹H NMR (400 MHz, DMSO-d₆) δ 10.17-10.12 (m, 1H), 8.35-8.31 (m, 1H), 8.02-7.91 (m, 1H), 7.82-7.67 (m, 2H), 7.30-7.23 (m, 1H), 7.20-7.04 (m, 3H), 6.98-6.82 (m, 1H), 6.76-6.51 (m, 2H), 5.60-5.38 (m, 1H), 4.14-3.97 (m, 1H), 3.30-2.73 (m, 4H), 2.62-2.48 (m, 1H), 2.46-1.57 (m, 9H), 1.04-0.95 (m, 9H), 0.77-0.66 (m, 6H).

### Step #6: (7aS,9R,11aS)-11a-Benzyl-9-hydroxy-5-oxo-9-trifluoromethyl-7a,8,9,10,11,11a-hexahydro-5H-dibenzo[a,c]cycloheptene-3-carboxylic acid (2-methyl-pyridin-3-yl)-amide (133, R²=Benzyl, R³=Trifluoromethyl, R⁶=2-Methylpyridin-3-yl)

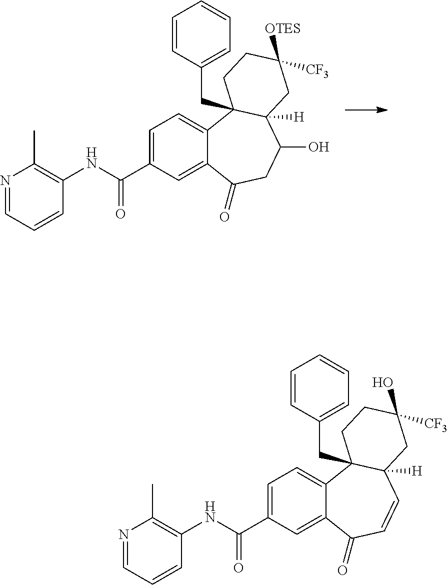

p-Toluenesulfonic acid monohydrate (0.170 g, 0.894 mmol) was added to a solution of (7aR,9R,11aS)-11a-benzyl-7-hydroxy-5-oxo-9-triethylsilanyloxy-9-trifluoromethyl-6,7,7a,8,9,10,11,11a-octahydro-5H-dibenzo[a,c]cycloheptene-3-carboxylic acid (2-methyl-pyridin-3-yl)-amide (132, R²=Benzyl, R³=Trifluoromethyl, R⁶=2-Methylpyridin-3-yl) (0.279 g, 0.427 mmol) and toluene (8.5 mL) under a nitrogen atmosphere. The solution was warmed to about 50° C. After about 30 min, the mixture was warmed to about 90° C. After about 1 h, the mixture was allowed to cool to rt. Saturated aqueous NaHCO₃ (10 mL) and water (10 mL) were added. The layers were separated and the aqueous phase was extracted with EtOAc (40 mL). The combined organics were washed with saturated aqueous NaCl (20 mL), dried over Na₂SO₄, filtered, and concentrated under reduced pressure to afford crude (7aS,9R,11aS)-11a-benzyl-9-hydroxy-5-oxo-9-trifluoromethyl-7a,8,9,10,11,11a-hexahydro-5H-dibenzo[a,c]cycloheptene-3-carboxylic acid (2-methyl-pyridin-3-yl)-amide (133, R²=Benzyl, R³=Trifluoromethyl, R⁶=2-Methylpyridin-3-yl) as a sticky yellow-tan solid. The crude product was used in the next step without further purification. LC/MS, method 3, Rₜ=2.10 min, MS m/z 522 (M+H)⁺. ¹H NMR (400 MHz, DMSO-d₆) δ 10.22 (s, 1H), 8.39-8.31 (m, 2H), 8.02 (dd, J=8.3, 2.2 Hz, 1H), 7.75 (dd, J=7.9, 1.5 Hz, 1H), 7.33 (d, J=8.7 Hz, 1H), 7.28 (dd, J=8.1, 4.7 Hz, 1H), 7.17-7.12 (m, 3H), 6.77-6.71 (m, 2H), 6.68 (dd, J=12.1, 7.2 Hz, 1H), 6.43 (d, J=12.1 Hz, 1H), 6.04 (s, 1H), 3.18-3.07 (m, 1H), 3.08 (d, J=13.3 Hz, 1H), 2.84 (d, J=13.4 Hz, 1H), 2.45 (s, 3H), 2.27-2.18 (m, 1H), 1.96-1.71 (m, 4H), 1.20-1.10 (m, 1H).

### Step #7: (7aR,9R,11aS)-11a-Benzyl-9-hydroxy-5-oxo-9-trifluoromethyl-6,7,7a,8,9,10,11,11a-octahydro-5H-dibenzo[a,c]cycloheptene-3-carboxylic acid (2-methyl-pyridin-3-yl)-amide (134, R²=Benzyl, R³=Trifluoromethyl, R⁶=2-Methylpyridin-3-yl)

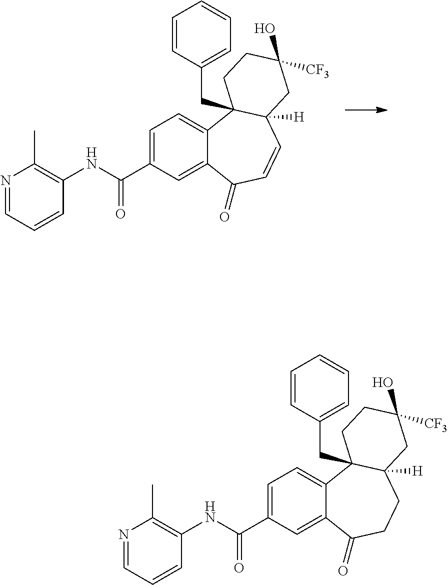

A mixture of (7aS,9R,11aS)-11a-benzyl-9-hydroxy-5-oxo-9-trifluoromethyl-7a,8,9,10,11,11a-hexahydro-5H-dibenzo[a,c]cycloheptene-3-carboxylic acid (2-methyl-pyridin-3-yl)-amide (133, R²=Benzyl, R³=Trifluoromethyl, R⁶=2-Methylpyridin-3-yl) (0.167 g, 0.320 mmol), Pd(OH)₂/C (20 wt %, wet, Degussa type) (0.050 g), and EtOAc (7.5 mL) was shaken under about 50 psi of H₂ at about 50° C. for about 2 h. Pd(OH)₂/C (20 wt %, wet, Degussa type) (0.100 g) was added. After shaking under about 50 psi of H₂ at about 50° C. for about 2 h, the mixture was filtered through Celite® rinsing with EtOAc (30 mL). The volatiles were removed under reduced pressure. The residue was purified on silica gel (12 g) using a gradient of 0.5-5% MeOH in DCM. The fractions containing product were combined and concentrated under reduced pressure to afford a white solid. The material was purified by HPLC: The gradient was 14.5% B for 3.5 min then 14.5-77.5% B over 9 min then 77.5-95.5% B over 1 min) Mobile phase A: 50 mM NH₄OAc in water, mobile phase B was HPLC grade MeCN. The column used for chromatography is 19×50 mm Waters Atlantis T3 OBD C18 column (5.0 μm particles). Detection methods are photodiode array (DAD) and Waters ZQ 2000 mass spectrometer. The organic volatiles were removed under reduced pressure. The mixture was frozen then lyophilized to provide a white solid. The material was slurried in water (5 mL) and then lyophilized to provide (7aR,9R,11aS)-11a-benzyl-9-hydroxy-5-oxo-9-trifluoromethyl-6,7,7a,8,9,10,11,11a-octahydro-5H-dibenzo[a,c]cycloheptene-3-carboxylic acid (2-methyl-pyridin-3-yl)-amide (134, R²=Benzyl, R³=Trifluoromethyl, R⁶=2-Methylpyridin-3-yl) (0.0558 g, 33%) as a white solid. LC/MS, method 2, Rₜ=2.08 min, MS m/z 524 (M+H)⁺. ¹H NMR (400 MHz, DMSO-d₆) δ 10.17 (s, 1H), 8.34 (dd, J=4.7, 1.6 Hz, 1H), 7.98 (d, J=2.1 Hz, 1H), 7.84 (dd, J=8.3, 2.1 Hz, 1H), 7.74 (dd, J=8.0, 1.6 Hz, 1H), 7.28 (dd, J=8.0, 4.7 Hz, 1H), 7.11-6.98 (m, 4H), 6.56-6.49 (m, 2H), 5.95 (s, 1H), 3.07-2.96 (m, 1H), 2.93 (d, J=12.9 Hz, 1H), 2.75 (d, J=13.2 Hz, 1H), 2.70-2.53 (m, 3H), 2.43 (s, 3H), 2.12-2.03 (m, 1H), 1.89-1.55 (m, 6H).

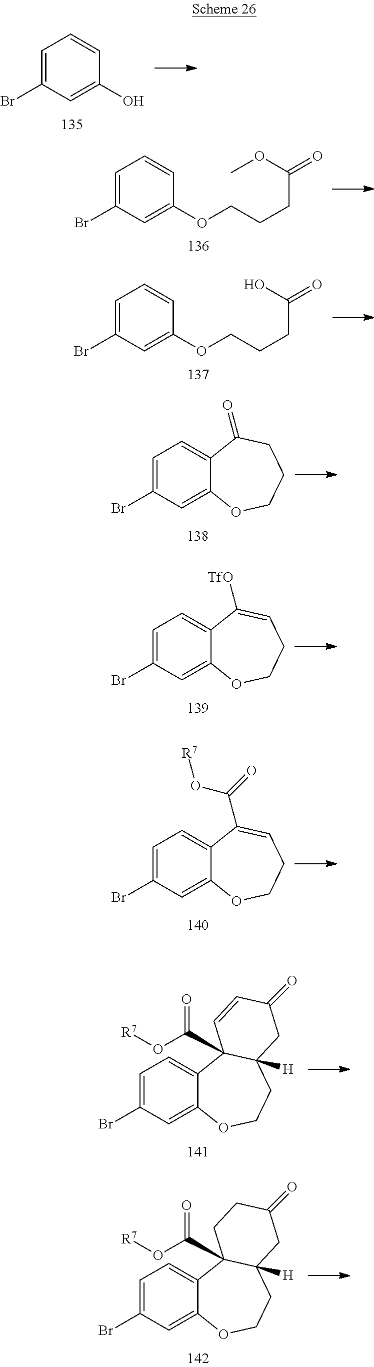

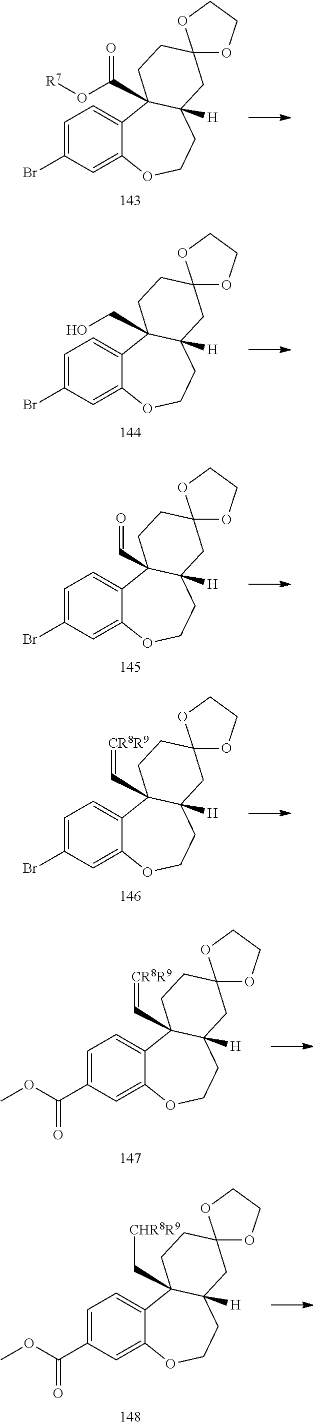

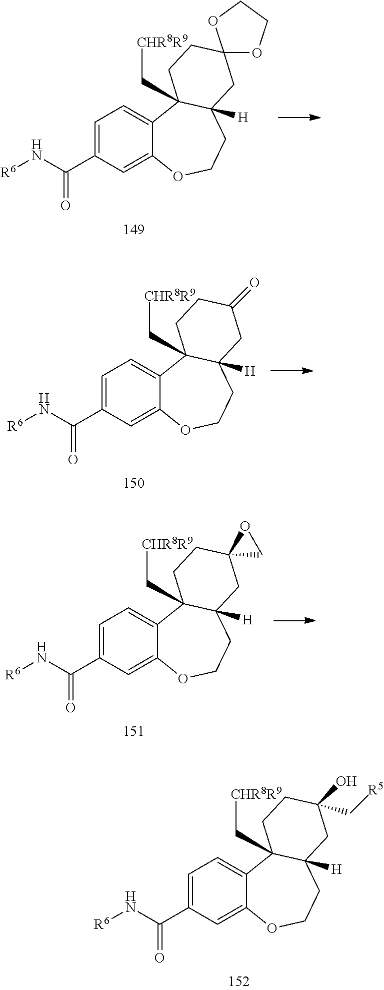

### Example #119

### (7aS,9R,11aR)-11a-Ethyl-9-hydroxy-N-(2-methylpyridin-3-yl)-9-propyl-6,7,7a,8,9,10,11,11a-octahydrodibenzo[b,d]oxepine-3-carboxamide; compound with (7aR,9S,11aS)-11a-ethyl-9-hydroxy-N-(2-methylpyridin-3-yl)-9-propyl-6,7,7a,8,9,10,11,11a-octahydrodibenzo[b,d]oxepine-3-carboxamide (152, R⁵=Ethyl, R⁸=H, R⁹=H)

### Step #1: Methyl 4-(3-bromophenoxy)butanoate (136)

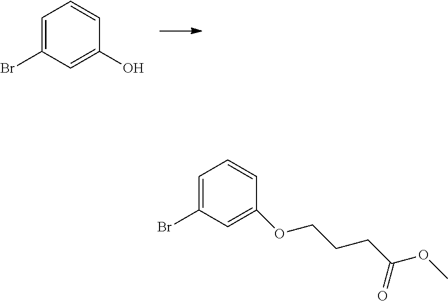

3-Bromophenol (13.7 g, 79.0 mmol) was dissolved in DMF (230 mL) then potassium carbonate (21.9 g, 158 mmol) and methyl 4-bromobutanoate (15.8 g, 87.0 mmol) were added. The mixture was stirred for about 30 min at rt then heated with stirring at about 95° C. for about 1 h. The mixture was cooled to about 15° C. and diluted with water (1 L). The mixture was extracted with DCM (250 mL). The layers were separated then the aqueous layer was extracted with DCM (150 mL). The combined organics were washed with water (2×375 mL) then dried over MgSO₄, filtered and concentrated under reduced pressure to give methyl 4-(3-bromophenoxy)butanoate (136) (25.8 g). LC/MS, method 3, Rₜ=2.61 min, MS m/z: 273, 275 (M+H)⁺. The crude product was used, as is, in the next step.

### Step #2: 4-(3-Bromophenoxy)butanoic acid (137)

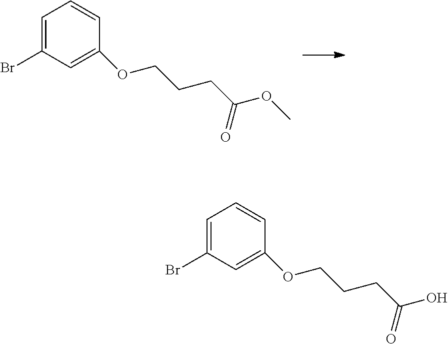

Methyl 4-(3-bromophenoxy)butanoate (136) (21.6 g, 79.0 mmol) was treated with 3 N aqueous sodium hydroxide (79 mL, 237 mmol) and MeOH (100 mL) then warmed to about 65° C. for about 30 min. The mixture was cooled to rt and concentrated under reduced pressure to remove most of the MeOH. The mixture was diluted with water (100 mL), acidified to about pH 2 with concentrated HCl then extracted with EtOAc (150 mL, then 75 mL). The combined organics were dried over MgSO₄, filtered and concentrated under reduced pressure to give 4-(3-bromophenoxy)butanoic acid (137) (20.3 g, 99%). LC/MS, method 3, Rₜ=2.15 min, MS m/z: 257, 259 (M−H)⁻. ¹H NMR (400 MHz, DMSO-d₆) δ 12.17 (bs, 1H), 7.23 (t, J=8.1 Hz, 1H), 7.16-7.09 (m, 2H), 6.94 (ddd, J=8.3, 2.4, 0.9 Hz, 1H), 4.00 (t, J=6.4 Hz, 2H), 2.37 (t, J=7.3 Hz, 2H), 1.96-1.80 (m, 2H).

### Step #3: 8-Bromo-3,4-dihydrobenzo[b]oxepin-5(211)-one (138)

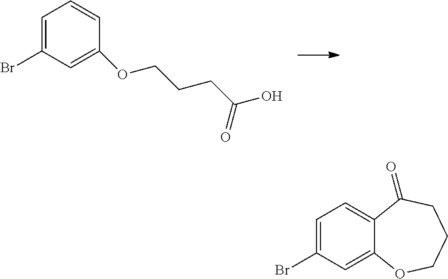

A round bottom flask with stir bar was charged with polyphosphoric acid (254 g). The material was heated to about 75° C. then 4-(3-bromophenoxy)butanoic acid (137) (20.0 g, 77.0 mmol) was added. The mixture was stirred at about 75° C. until the materials were mixed. The mixture was heated to about 100° C. for about 30 min then cooled in an ice bath. Water (250 mL) was slowly added to the reaction mixture then the mixture was added to water (250 mL). After stirring for about 30 min the mixture was extracted with EtOAc (2×150 mL) then the combined organics were washed with 1 N aqueous NaOH (250 mL) and saturated aqueous NaCl (200 mL). The organic solution was dried over MgSO₄, filtered though a pad of Celite® then concentrated under reduced pressure. The crude product was purified on silica gel (220 g) using a gradient from 0-30% EtOAc in heptane. Fractions containing the product were combined and concentrated to yield 8-bromo-3,4-dihydrobenzo[b]oxepin-5(2H)-one (138) (13.9 g, 75%). LC/MS, method 3, 11, =2.40 min, No parent ion. ¹H NMR (400 MHz, DMSO-d₆) δ 7.56 (d, J=8.0 Hz, 1H), 7.38-7.33 (m, 2H), 4.24 (t, J=6.5 Hz, 2H), 2.79 (t, J=6.9 Hz, 2H), 2.20-2.08 (m, 2H).

### Step #4: 8-Bromo-2,3-dihydrobenzo[b]oxepin-5-yl trifluoromethanesulfonate (139)

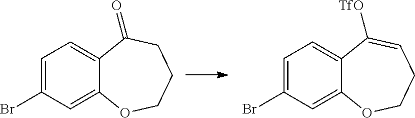

8-Bromo-3,4-dihydrobenzo[b]oxepin-5(2H)-one (138) (13.9 g, 57.6 mmol) in DCM (225 mL) was treated with Na₂CO₃ (18.3 g, 173 mmol) and the mixture was cooled to about 0° C. The stirred mixture was treated with trifluoromethanesulfonic anhydride (40 g, 142 mmol) then warmed to rt and stirred for about 16 h. Water (300 mL) was added, the mixture was stirred for about 30 min and then the layers were separated. The organic layer was dried over MgSO₄, filtered and concentrated under reduced pressure to an oil which solidified upon standing. The material was dissolved in EtOAc (10 mL) and heptane (90 mL) with heating. The mixture was cooled in an ice/water bath and the solids were collected by filtration and washed with heptane (10 mL). The material was dried under reduced pressure at about 70° C. to give a first lot of 8-bromo-2,3-dihydrobenzoibioxepin-5-yl trifluoromethanesulfonate (139) (13.1 g, 61%). The filtrate was concentrated under reduced pressure to a solid which was purified on silica gel (330 g) using a gradient from 0-35% EtOAc in heptane. Fractions containing the product were combined and concentrated under reduced pressure to yield a second lot of 8-bromo-2,3-dihydrobenzoibioxepin-5-yl trifluoromethanesulfonate (139) (5.74 g, 27%). LC/MS, method 3, Rₜ=2.96 min, No parent ion. ¹H NMR (400 MHz, DMSO-d₆) δ 7.46-7.35 (m, 3H), 6.47 (t, J=4.9 Hz, 1H), 4.20 (t, J=5.2 Hz, 2H), 2.77-2.73 (m, 2H)

### Step #5: Methyl 8-bromo-2,3-dihydrobenzo[b]oxepine-5-carboxylate (140, R⁷=Methyl)

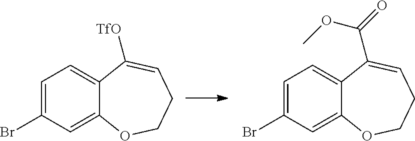

A solution of 8-bromo-2,3-dihydrobenzo[b]oxepin-5-yl trifluoromethanesulfonate (139) (17.4 g, 46.6 mmol) in DMF (175 mL) was degassed by stirring under ˜15 mm Hg vacuum for about 15 min. The flask was filled with carbon monoxide using a balloon then 1,3-bis(diphenylphosphino)propane (0.962 g, 2.33 mmol), diacetoxypalladium (0.523 g, 2.33 mmol), MeOH (87 mL) and triethylamine (14.16 g, 140 mmol) were added. The flask was briefly evacuated under reduced pressure then the flask was filled with carbon monoxide with a balloon. This was repeated two more times then the mixture was heated under an atmosphere of carbon monoxide at about 80° C. for about 2 h with stirring. The mixture was cooled to rt then concentrated under reduced pressure and partitioned between water (250 mL) and EtOAc (150 mL). The organic solution was washed with saturated aqueous NaCl (50 mL), dried over MgSO₄, then filtered and concentrated under reduced pressure. The residue was purified on silica gel (120 g) using a gradient from 0-35% EtOAc in heptane. Pure product fractions were combined and concentrated to give methyl 8-bromo-2,3-dihydrobenzo[b]oxepine-5-carboxylate (140, R⁷=Methyl) (5.52 g, 42%). LC/MS, method 3, Rₜ=2.52 min, MS m/z 300, 302 (M+H₂O)⁺. ¹H NMR (400 MHz, DMSO-d₆) δ 7.37-7.33 (m, 1H), 7.31-7.27 (m, 2H), 7.22 (t, J=6.4 Hz, 1H), 4.40 (t, J=6.1 Hz, 2H), 3.75 (s, 3H), 2.48-2.44 (m, 2H).

### Step #6: (7aS,11aR)-Methyl 3-bromo-9-oxo-6,7,7a,8,9,11a-hexahydrodibenzo[b,d]oxepine-11a-carboxylate; compound with (7aR,11aS)-methyl 3-bromo-9-oxo-6,7,7a,8,9,11a-hexahydrodibenzo[b,d]oxepine-11a-carboxylate (141, R⁷=Methyl)

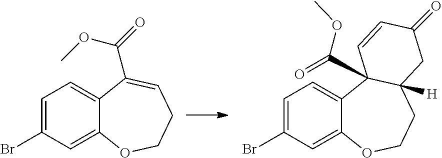

A steel pressure vessel with stirrer was charged with methyl 8-bromo-2,3-dihydrobenzo[b]oxepine-5-carboxylate (140, R⁷=Methyl) (5.81 g, 20.5 mmol), toluene (25 mL) and (E)-(4-methoxybuta-1,3-dien-2-yloxy)trimethylsilane (17.7 g, 103 mmol). The vessel was sealed then heated with stirring at about 125° C. for about 72 h. The mixture was concentrated under reduced pressure then the material was treated with THF (75 mL) and 6 N aqueous HCl (14 mL). The mixture was stirred at rt for about 6 h. Water (250 mL) was added then the mixture was extracted with EtOAc (150 mL, then 100 mL). The combined organics were washed with saturated aqueous NaCl (100 mL), dried over MgSO₄, filtered and the concentrated under reduced pressure. The material was purified on silica gel (220 g) using a gradient from 0-50% EtOAc in heptane. Fractions containing the product were combined and concentrated to give (7aS,11aR)-methyl 3-bromo-9-oxo-6,7,7a,8,9,11a-hexahydrodibenzo[b,d]oxepine-11a-carboxylate; compound with (7aR11aS)-methyl 3-bromo-9-oxo-6,7,7a,8,9,11a-hexahydrodibenzo[b,d]oxepine-11a-carboxylate (141, R⁷=Methyl) (5.32 g, 74%). LC/MS, method 3, Rₜ=2.44 min, No parent ion. ¹H NMR (400 MHz, DMSO-d₆) δ 7.36 (dd, J=8.2, 2.1 Hz, 1H), 7.27 (d, J=2.1 Hz, 1H), 7.03 (d, J=8.2 Hz, 1H), 6.94 (d, J=10.1 Hz, 1H), 6.30 (d, J=10.1 Hz, 1H), 4.15-4.08 (m, 1H), 3.96-3.91 (m, 1H), 3.61 (s, 3H), 3.26-3.17 (m, 1H), 2.27-2.17 (m, 1H), 1.97-1.81 (m, 2H), 1.27-1.12 (m, 1H).

### Step #7: (7aS,11aS)-Methyl 3-bromo-9-oxo-6,7,7a,8,9,10,11,11a-octahydrodibenzo[b,d]oxepine-11a-carboxylate; compound with (7aR,11aR)-methyl 3-bromo-9-oxo-6,7,7a,8,9,10,11,11a-octahydrodibenzo[b,d]oxepine-11a-carboxylate (142, R⁷=Methyl)

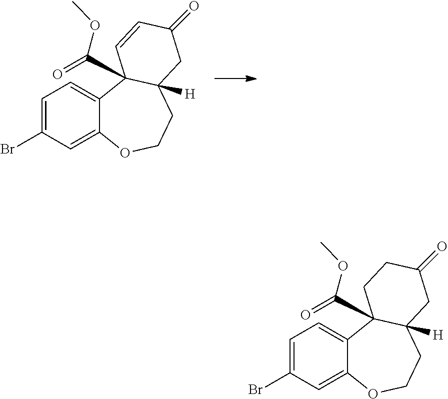

A flask with stir bar was charged with (7aS,11aR)-methyl 3-bromo-9-oxo-6,7,7a,8,9,11a-hexahydrodibenzo[b,d]oxepine-11a-carboxylate; compound with (7aR,11aS)-methyl 3-bromo-9-oxo-6,7,7a,8,9,11a-hexahydrodibenzo[b,d]oxepine-11a-carboxylate (141, R⁷=Methyl) (4.15 g, 11.8 mmol), NaHCO₃ (4.96 g, 59.1 mmol), Aliquot™ 336 [Henkel] (1.43 g, 3.55 mmol), toluene (80 mL) and water (80 mL). The mixture was heated to about 100° C. Sodium hydrosulfite (tech ˜85%) (6.95 g, 39.9 mmol) was added in three roughly equal portions; one when the mixture was heated to about 100° C., the second after about 5 min and the final portion after about 25 min. About 5 min after the last sodium hydrosulfite addition, the mixture was cooled to rt and transferred to a separatory funnel The layers were separated then the organic layer was washed with saturated aqueous NaCl (20 mL), dried over MgSO₄ and filtered, rinsing with EtOAc (75 mL). The filtrate was concentrated under reduced pressure. The material was purified on silica gel (80 g) using a gradient of 0-100% EtOAc in heptane. Fractions containing the product were combined and concentrated to give (7aS,11aS)-methyl 3-bromo-9-oxo-6,7,7a,8,9,10,11,11a-octahydrodibenzo[b,d]oxepine-11a-carboxylate; compound with (7aR,11aR)-methyl 3-bromo-9-oxo-6,7,7a,8,9,10,11,11a-octahydrodibenzo[b,d]oxepine-11a-carboxylate (142, R⁷=Methyl) (3.29 g, 79%). LC/MS, method 3, Rₜ=2.36 min, MS m/z: 353, 355 (M+H)⁺. ¹H NMR (400 MHz, CDCl₃) δ 7.31 (dd, J=8.4, 2.1 Hz, 1H), 7.23 (d, J=2.1 Hz, 1H), 7.17 (d, J=8.5 Hz, 1H), 4.18-4.12 (m, 1H), 3.97-3.91 (m, 1H), 3.71 (s, 3H), 3.14-3.07 (m, 1H), 2.69-2.47 (m, 3H), 2.39-2.18 (m, 4H), 1.70-1.59 (m, 1H).

### Step #8: (7aS,11aS)-Methyl 3-bromo-7,7a,8,10,11,11a-hexahydro-6H-spiro[dibenzo[b,d]oxepine-9,2′-[1,3]dioxolane]-11a-carboxylate; compound with (7aR,11aR)-methyl 3-bromo-7,7a,8,10,11,11a-hexahydro-6H-spiro[dibenzo[b,d]oxepine-9,2′-[1,3]dioxolane]-11a-carboxylate (143, R⁷=Methyl)

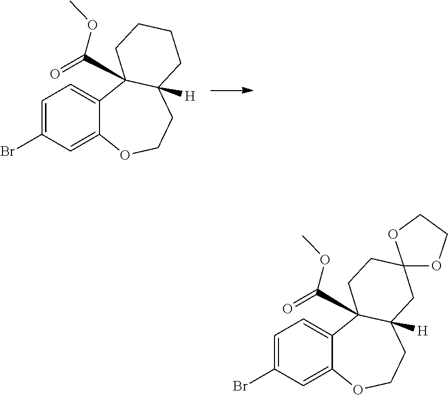

A flask equipped with a stir bar, Dean-Stark apparatus, condensor and nitrogen line was charged with (7aS,11aS)-methyl 3-bromo-9-oxo-6,7,7a,8,9,10,11,11a-octahydrodibenzo[b,d]oxepine-11a-carboxylate; compound with (7aR,11aR)-methyl 3-bromo-9-oxo-6,7,7a,8,9,10,11,11a-octahydrodibenzo[b,d]oxepine-11a-carboxylate (142, R⁷=Methyl) (4.06 g, 11.49 mmol), toluene (100 mL), ethane-1,2-diol (2.14 g, 34.5 mmol) and 4-methylbenzenesulfonic acid hydrate (0.164 g, 0.862 mmol). The mixture was heated at reflux for about 1 h, removing water in the Dean-Stark trap. The mixture was cooled to rt then Na₂CO₃ (˜2 g) was added. The mixture was stirred for about 10 min, saturated aqueous NaHCO₃ (5 mL) was added and the mixture was diluted with water (100 mL). The layers were separated then the organic layer was washed with water (100 mL) and saturated aqueous NaCl (50 mL). The organic solution was dried over MgSO₄, filtered and concentrated under reduced pressure to give (7aS,11aS)-methyl 3-bromo-7,7a,8,10,11,11a-hexahydro-6H-spiro[dibenzo[b,d]oxepine-9,2′-[1, 3]dioxolane]-11a-carboxylate; compound with (7aR,11aR)-methyl 3-bromo-7,7a,8,10,11,11a-hexahydro-6H-spiro[dibenzo[b,d]oxepine-9,2′-[1,3]dioxolane]-11a-carboxylate (143, R⁷=Methyl) (4.63 g, 101%). LC/MS, method 3, Rₜ=2.72 min, MS m/z: 397, 399 (M+H)⁺. ¹H NMR (400 MHz, CDCl₃) δ 7.32-7.15 (m, 3H), 4.23-4.14 (m, 1H), 4.06-3.90 (m, 4H), 3.87-3.74 (m, 1H), 3.65 (s, 3H), 3.03-2.95 (m, 1H), 2.55-2.41 (m, 1H), 2.36-2.28 (m, 1H), 2.24-2.16 (m, 1H), 1.94-1.79 (m, 2H), 1.67-1.44 (m, 3H)

### Step #9: ((7aS,11aS)-3-Bromo-7,7a,8,10,11,11a-hexahydro-6H-spiro[dibenzo[b,d]oxepine-9,2′-[1,3]dioxolane]-11a-yl)methanol; compound with ((7aR,11aR)-3-bromo-7,7a,8,10,11,11a-hexahydro-6H-spiro[dibenzo[b,d]oxepine-9,2′-[1,3]dioxolane]-11a-yl)methanol (144)

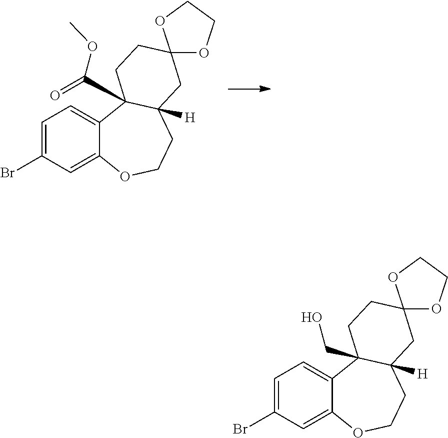

A flask equipped with stir bar, septum, thermometer and nitrogen line was charged with (7aS,11aS)-methyl 3-bromo-7,7a,8,10,11,11a-hexahydro-6H-spiro[dibenzo[b,d]oxepine-9,2′-[1,3]dioxolane]-11a-carboxylate; compound with (7aR,11aR)-methyl 3-bromo-7,7a,8,10,11,11a-hexahydro-6H-spiro[dibenzo[b,d]oxepine-9,2′-[1,3]dioxolane]-11a-carboxylate (143, R⁷=Methyl) (4.66 g, 11.7 mmol) dissolved in THF (30 mL). The mixture was cooled to an internal temperature of about −65° C. and LiAlH₄ (1 M in THF, 13 mL, 13 mmol) was added over about 20 min, maintaining the reaction temperature below about −60° C. After about 30 min, the reaction mixture was warmed to about 0° C. for about 5 min and then cooled to about −60° C. 1 N aqueous NaOH (6 mL) then EtOAc (50 mL) were added. The mixture was warmed to rt and diluted with water (100 mL), EtOAc (50 mL) and heptane (25 mL). The layers were separated and the aqueous layer was extracted with 50% EtOAc in heptane (2×50 mL). The combined organics were dried over MgSO₄, filtered and concentrated under reduced pressure. The material was purified on silica gel (80 g) using a gradient of 25-100% EtOAc in heptane. Fractions containing product were combined and concentrated to give ((7aS,11aS)-3-bromo-7,7a,8,10,11,11a-hexahydro-6H-spiro[dibenzo[b,d]oxepine-9,2′-[1,3]dioxolane]-11a-yl)methanol; compound with ((7aR,11aR)-3-bromo-7,7a,8,10,11,11a-hexahydro-6H-spiro[dibenzo[b,d]oxepine-9,2′-[1,3]dioxolane]-11a-yl)methanol (144) (3.88 g, 90%). LC/MS, method 3, Rₜ=2.24 min, MS m/z: 369, 371 (M+H)⁺. ¹H NMR (400 MHz, CDCl₃) δ 7.23-7.19 (m, 2H), 7.16 (d, J=8.5 Hz, 1H), 4.26-4.21 (m, 1H), 4.04-3.88 (m, 5H), 3.81-3.69 (m, 2H), 2.60-2.51 (m, 1H), 2.43-2.37 (m, 2H), 1.97-1.79 (m, 3H), 1.73-1.66 (m, 1H), 1.61-1.54 (m 1H), 1.40-1.34 (m, 1H).

### Step #10: (7aS,11aS)-3-Bromo-7,7a,8,10,11,11a-hexahydro-6H-spiro[dibenzo[b,d]oxepine-9,2′-[1,3]dioxolane]-11a-carbaldehyde; compound with (7aR,11aR)-3-bromo-7,7a,8,10,11,11a-hexahydro-6H-spiro[dibenzo[b,d]oxepine-9,2′-[1,3]dioxolane]-11a-carbaldehyde (145)

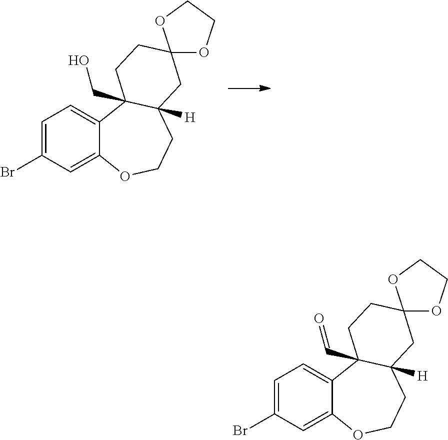

((7aS,11aS)-3-Bromo-7,7a,8,10,11,11a-hexahydro-6H-spiro[dibenzo[b,d]oxepine-9,2′-[1,3]dioxolane]-11a-yl)methanol; compound with ((7aR,11aR)-3-bromo-7,7a,8,10,11,11a-hexahydro-6H-spiro[dibenzo[b,d]oxepine-9,2′-[1,3]dioxolane]-11a-yl)methanol (144) (3.88 g, 10.5 mmol) in DCM (125 mL) was treated with Dess-Martin periodinane (4.46 g, 10.5 mmol). The mixture was stirred at rt for about 90 min. The mixture was diluted with Et₂O (200 mL) then filtered, washing with Et₂O (50 mL), then the filtrate was concentrated under reduced pressure. The material was triturated with 50% EtOAc in heptane (100 mL) then filtered and the cake washed with 50% EtOAc in heptane (25 mL). The filtrate was concentrated under reduced pressure then the material was purified on silica gel (80 g) using a gradient from 0-60% EtOAc in heptane. Fractions containing product were combined and concentrated to give (7aS,11aS)-3-bromo-7,7a,8,10,11,11a-hexahydro-6H-spiro[dibenzo[b,d]oxepine-9,2′-[1,3]dioxolane]-11a-carbaldehyde; compound with (7aR,11aR)-3-bromo-7,7a,8,10, 11,11a-hexahydro-6H-spiro[dibenzo[b,d]oxepine-9,2′-[1,3]dioxolane]-11a-carbaldehyde (145) (3.10 g, 80%). LC/MS, method 3, Rₜ=2.54 min, MS m/z: 369, 371 (M+H)⁺; ¹H NMR (400 MHz, CDCl₃) δ 9.49 (s, 1H), 7.35-7.19 (m, 3H), 4.17-4.09 (m, 1H), 4.03-3.92 (m, 4H), 3.83-3.80 (m, 1H), 2.87-2.80 (m, 1H), 2.31-2.17 (m, 2H), 2.03-1.84 (m, 3H), 1.66-1.46 (m, 3H).

### Step #11: (7aS,11aS)-3-Bromo-11a-vinyl-7,7a,8,10,11,11a-hexahydro-6H-Spiro[dibenzo[b,d]oxepine-9,2′-[1,3]dioxolane]; compound with (7aR,11aR)-3-bromo-11a-vinyl-7,7a,8,10,11,11a-hexahydro-6H-spiro[dibenzo[b,d]oxepine-9,2′-[1,3]dioxolane] (146, R⁸=H, R⁹=H)

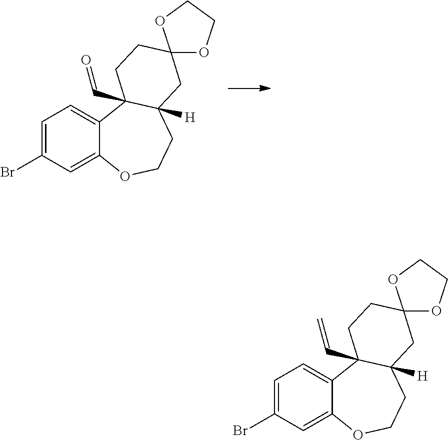

A round bottom flask with stir bar and nitrogen line was charged with DMSO (11 mL) and sodium hydride (60 wt % dispersion in mineral oil, 0.675 g, 16.9 mmol). The mixture was heated to about 60° C. for about 1 h, then cooled to rt. The mixture was diluted with THF (11 mL) and methyltriphenylphosphonium bromide (6.03 g, 16.9 mmol) was added. The mixture was stirred for about 30 min then (7aS,11aS)-3-bromo-7,7a,8,10,11,11a-hexahydro-6H-spiro[dibenzo[b,d]oxepine-9,2′-[1,3]dioxolane]-11a-carbaldehyde; compound with (7aR,11aR)-3-bromo-7,7a,8,10,11,11a-hexahydro-6H-spiro[dibenzo[b,d]oxepine-9,2′-[1,3]dioxolane]-11a-carbaldehyde (145) (3.10 g, 8.44 mmol) in THF (11 mL) was added over about 10 min. The mixture was warmed to rt for about 18 h. Water (75 mL) was added and the mixture was extracted with Et₂O (3×50 mL). The combined organics were dried over MgSO₄, filtered and concentrated under reduced pressure. The material was purified on silica gel (80 g) using a gradient of 0-40% EtOAc in heptane followed by a second purification on silica gel (40 g) using a gradient of 0-40% EtOAc in heptane. Fractions containing product were combined and concentrated to give the (7aS,11aS)-3-bromo-11a-vinyl-7,7a,8,10,11,11a-hexahydro-6H-spiro[dibenzo[b,d]oxepine-9,2′-[1,3]dioxolane]; compound with (7aR,11aR)-3-bromo-11a-vinyl-7,7a,8,10,11,11a-hexahydro-6H-spiro[dibenzo[b,d]oxepine-9,2′-[1,3]dioxolane] (146, R⁸=H, R⁹=H) (2.95 g, 96%). LC/MS, method 3, Rₜ=2.95 min, No parent ion; ¹H NMR (400 MHz, CDCl₃) δ 7.20-7.10 (m, 3H), 5.97 (dd, J=17.3, 10.6 Hz, 1H), 5.02-4.96 (m, 1H), 4.54 (dd, J=17.3, 1.1 Hz, 1H), 4.18-4.08 (m, 1H), 4.00-3.88 (m, 4H), 3.87-3.78 (m, 1H), 2.45-2.36 (m, 1H), 2.32-2.26 (m, 1H), 2.24-2.18 (m, 1H), 1.95-1.81 (m, 1H), 1.80-1.67 (m, 3H), 1.63-1.56 (m, 1H), 1.49-1.42 (m, 1H).

### Step #12: (7aS,11aS)-Methyl 11a-vinyl-7,7a,8,10,11,11a-hexahydro-6H-spiro[dibenzo[b,d]oxepine-9,2′-[1,3]dioxolane]-3-carboxylate; compound with (7aR,11aR)-methyl 11a-vinyl-7,7a,8,10,11,11a-hexahydro-6H-spiro[dibenzo[b,d]oxepine-9,2′-[1,3]dioxolane]-3-carboxylate (147, R⁸=H, R⁹=H)

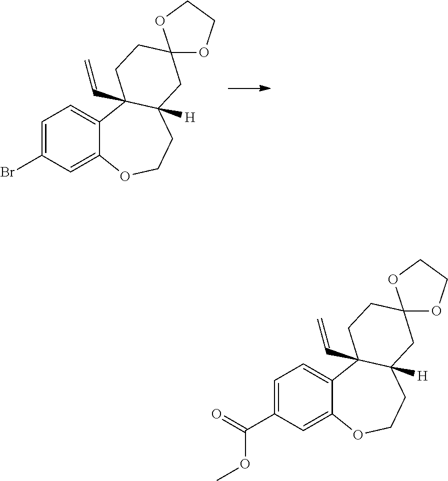

A round bottom flask with stir bar was charged with (7aS,11aS)-3-bromo-11a-vinyl-7,7a,8,10,11,11a-hexahydro-6H-spiro[dibenzo[b,d]oxepine-9,2′-[1,3]dioxolane]; compound with (7aR,11aR)-3-bromo-11a-vinyl-7,7a,8,10,11,11a-hexahydro-6H-spiro[dibenzo[b,d]oxepine-9,2′-[1,3]dioxolane] (146, R⁸=H, R⁹=H) (2.75 g, 7.53 mmol) and DMF (60 mL). The mixture was degassed by stirring under ˜15 mm Hg vacuum for about 15 min. The flask was filled with carbon monoxide with a balloon then it was charged with Pd₂(dba)₃ (0.207 g, 0.226 mmol), Xantphos (0.436 g, 0.753 mmol), TEA (3.05 g, 30.1 mmol) and methanol (2.89 g, 90 mmol). The flask was briefly evacuated under reduced pressure then the flask was filled with carbon monoxide using a balloon. This was repeated two more times then the mixture was heated under an atmosphere of carbon monoxide to about 90° C. for about 14 h with stirring. The mixture was cooled to rt then diluted with water (500 mL). The mixture was extracted with EtOAc (250 mL then 100 mL) and then the combined organics were washed with water (250 mL) and saturated aqueous NaCl (100 mL). The organic solution was dried over MgSO₄, filtered and concentrated under reduced pressure. The material was purified on silica gel (40 g) using a gradient of 0-50% EtOAc in heptane. Fractions containing the product were combined and concentrated to give (7aS,11aS)-methyl 11a-vinyl-7,7a,8,10, 11,11a-hexahydro-6H-spiro[dibenzo[b,d]oxepine-9,2′-[1,3]dioxolane]-3-carboxylate; compound with (7aR,11aR)-methyl 11a-vinyl-7,7a,8,10,11,11a-hexahydro-6H-spiro[dibenzo[b,d]oxepine-9,2′-[1,3]dioxolane]-3-carboxylate (147, R⁸=H, R⁹=H) (2.03 g, 78%). LC/MS, method 3, Rₜ=2.60 min, MS m/z: 345 (M+H)⁺; ¹H NMR (400 MHz, CDCl₃) δ 7.74 (dd, J=8.3, 1.9 Hz, 1H), 7.66 (d, J=1.9 Hz, 1H), 7.37 (d, J=8.4 Hz, 1H), 6.04 (dd, J=17.3, 10.6 Hz, 1H), 5.02 (dd, J=10.6, 1.0 Hz, 1H), 4.55 (dd, J=17.3, 1.0 Hz, 1H), 4.23-4.17 (m, 1H), 4.02-3.94 (m, 4H), 3.93 (s, 3H), 3.89-3.83 (m, 1H), 2.52-2.44 (m, 1H), 2.39-2.27 (m, 2H), 2.01-1.90 (m, 1H), 1.84-1.71 (m, 3H), 1.69-1.62 (m, 1H), 1.53-1.46 (m, 1H).

### Step #13: (7aS,11aR)-Methyl 11a-ethyl-7,7a,8,10,11,11a-hexahydro-6H-spiro[dibenzo[b,d]oxepine-9,2′-[1,3]dioxolane]-3-carboxylate; compound with (7aR,11aS)-methyl 11a-ethyl-7,7a,8,10,11,11a-hexahydro-6H-spiro[dibenzo[b,d]oxepine-9,2′-[1,3]dioxolane]-3-carboxylate (148, R⁸=H, R⁹=H)

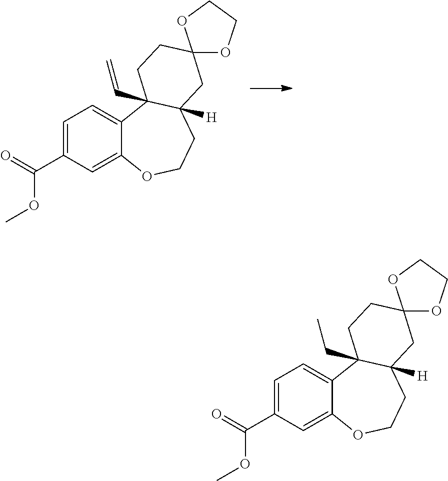

(7aS,11aS)-Methyl 11a-vinyl-7,7a,8,10,11,11a-hexahydro-6H-spiro[dibenzo[b,d]oxepine-9,2′-[1,3]dioxolane]-3-carboxylate; compound with (7aR,11aR)-methyl 11a-vinyl-7,7a,8,10,11,11a-hexahydro-6H-spiro[dibenzo[b,d]oxepine-9,2′-[1,3]dioxolane]-3-carboxylate (147, R⁸=H, R⁹=H) (2.03 g, 5.89 mmol) in EtOAc (60 mL) was treated with platinum (IV) oxide (0.200 g, 0.881 mmol) then the flask was evacuated and filled with hydrogen using a balloon. This was repeated 3 times then the mixture was stirred under an atmosphere of hydrogen for about 2 h. The catalyst was removed by filtration through a pad of Celite® then the filtrate was concentrated under reduced pressure to give (7aS,11aR)-methyl 11a-ethyl-7,7a,8,10, 11,11a-hexahydro-6H-spiro[dibenzo[b,d]oxepine-9,2′-[1,3]dioxolane]-3-carboxylate; compound with (7aR,11aS)-methyl 11a-ethyl-7,7a,8,10,11,11a-hexahydro-6H-spiro[dibenzo[b,d]oxepine-9,2′-[1,3]dioxolane]-3-carboxylate (148, R⁸=H, R⁹=H) (1.98 g, 97%). LC/MS, method 3, Rₜ=2.71 min, MS m/z: 347 (M+H)⁺; ¹H NMR (400 MHz, CDCl₃) δ 7.68 (dd, J=8.4, 1.9 Hz, 1H), 7.61 (d, J=1.9 Hz, 1H), 7.27 (d, J=8.4 Hz, 1H), 4.25-4.20 (m, 1H), 4.01-3.91 (m, 4H), 3.89 (s, 3H), 3.73-3.64 (m, 1H), 2.68-2.63 (m, 1H), 2.40-2.38 (m, 1H), 2.28-2.13 (m, 2H), 1.94-1.74 (m, 2H), 1.71-1.57 (m, 2H), 1.54-1.45 (m, 2H), 1.40-1.31 (m, 1H), 0.61 (t, J=7.2 Hz, 3H).

### Step #14: (7aS,11aR)-11a-Ethyl-N-(2-methylpyridin-3-yl)-7,7a,8,10,11,11a-hexahydro-6H-spiro[dibenzo[b,d]oxepine-9,2′-[1,3]dioxolane]-3-carboxamide; compound with (7aR,11aS)-11a-ethyl-N-(2-methylpyridin-3-yl)-7,7a,8,10,11,11a-hexahydro-6H-spiro[dibenzo[b,d]oxepine-9,2′-[1,3]dioxolane]-3-carboxamide (149, R⁶=2-Methylpyridin-3-yl, R⁸H, R⁹=H)

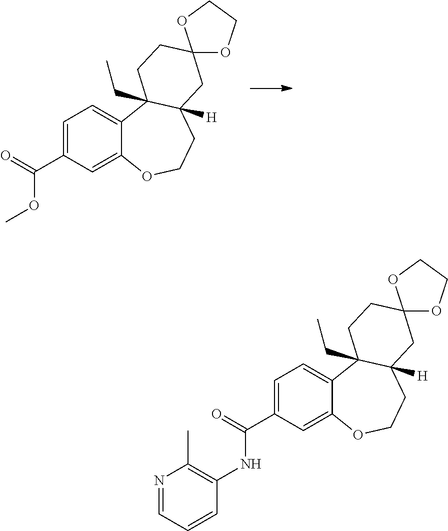

A round bottom flask with stir bar and nitrogen line was charged with (7aS,11aR)-methyl 11a-ethyl-7,7a,8,10,11,11a-hexahydro-6H-spiro[dibenzo[b,d]oxepine-9,2′-[1,3]dioxolane]-3-carboxylate; compound with (7aR,11aS)-methyl 11a-ethyl-7,7a,8,10,11,11a-hexahydro-6H-spiro[dibenzo[b,d]oxepine-9,2′-[1,3]dioxolane]-3-carboxylate (148, R⁸=H, R⁹=H) (2.04 g, 5.89 mmol), toluene (60 mL) and 2-methylpyridin-3-amine (0.764 g, 7.07 mmol). The mixture was stirred for about 15 min at rt then cooled to about 0° C. and treated with LiHMDS (1 M solution in THF, 17.7 mL, 17.7 mmol). The mixture was stirred at about 0° C. for about 15 min then treated with saturated aqueous NaHCO₃ (50 mL) and water (25 mL). The mixture was warmed to rt with stirring. The layers were separated then the aqueous layer was extracted with EtOAc (2×25 mL). The combined organics were washed with saturated aqueous NaCl (30 mL) then dried over MgSO₄, filtered and concentrated under reduced pressure. The material was purified on silica gel (40 g) using a gradient from 0-10% MeOH in DCM. Pure product fractions were combined and concentrated to give (7aS,11aR)-11a-ethyl-N-(2-methylpyridin-3-yl)-7,7a,8,10,11,11a-hexahydro-6H-spiro[dibenzo[b,d]oxepine-9,2′-[1,3]dioxolane]-3-carboxamide; compound with (7aR,11aS)-11a-ethyl-N-(2-methylpyridin-3-yl)-7,7a,8,10,11,11a-hexahydro-6H-spiro[dibenzo[b,d]oxepine-9,2′-[1,3]dioxolane]-3-carboxamide (149, R⁶=2-Methylpyridin-3-yl, R⁸=H, R⁹=H) (2.55 g, 102%) as a foam. LC/MS, method 3, Rₜ=2.29 min, m/z: 423 (M+H)⁺; NMR indicates presence of ˜4 wt % DCM. ¹H NMR (400 MHz, CDCl₃) δ 8.40 (d, J=8.2 Hz, 1H), 8.33 (dd, J=4.8, 1.5 Hz, 1H), 7.64 (s, 1H), 7.55 (dd, J=8.2, 2.1 Hz, 1H), 7.46 (d, J=2.1 Hz, 1H), 7.36 (d, J=8.3 Hz, 1H), 7.23 (dd, J=8.2, 4.8 Hz, 1H), 4.29-4.24 (m, 1H), 4.03-3.85 (m, 4H), 3.77-3.71 (m, 1H), 2.74-2.65 (m, 1H), 2.62 (s, 3H), 2.42-2.36 (m, 1H), 2.28-2.15 (m, 2H), 1.96-1.75 (m, 2H), 1.75-1.46 (m, 4H), 1.39-1.35 (m, 1H), 0.65 (t, J=7.5 Hz, 3H).

### Step #15: (7aS,11aR)-11a-Ethyl-N-(2-methylpyridin-3-yl)-9-oxo-6,7,7a,8,9,10,11,11a-octahydro dibenzo[b,d]oxepine-3-carboxamide; compound with (7aR,11aS)-11a-ethyl-N-(2-methylpyridin-3-yl)-9-oxo-6,7,7a,8,9,10,11,11a-octahydrodibenzo[b,d]oxepine-3-carboxamide (150, R⁶=2-methylpyridin-3-yl, R⁸=H, R⁹=H)

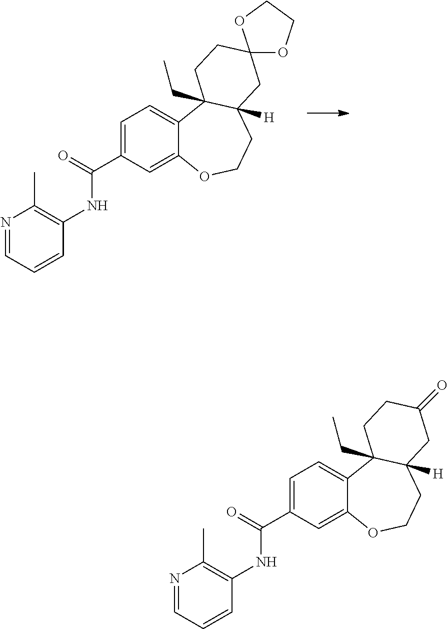

(7aS,11aR)-11a-Ethyl-N-(2-methylpyridin-3-yl)-7,7a,8,10,11,11a-hexahydro-6H-spiro[dibenzo[b,d]oxepine-9,2′-[1,3]dioxolane]-3-carb oxamide; compound with (7aR,11aS)-11a-ethyl-N-(2-methylpyridin-3-yl)-7,7a,8,10,11,11a-hexahydro-6H-spiro[dibenzo[b,d]oxepine-9,2′-[1,3]dioxolane]-3-carboxamide (149, R⁶=2-methylpyridin-3-yl, R⁸=H, R⁹=H) (2.55 g, 6.04 mmol) was dissolved in THF (60 mL) and treated with 6 N aqueous HCl (6.0 mL, 36 mmol). The mixture was stirred at rt for about 16 h. Water (25 mL) was added then after about 10 min the mixture was added to a stirred solution of saturated aqueous NaHCO₃ (200 mL). The mixture was transferred to a separatory funnel and water (25 mL) and EtOAc (100 mL) were added. The layers were separated then the aqueous layer was extracted with EtOAc (50 mL). The combined organics were dried over MgSO₄, filtered and concentrated under reduced pressure to give the (7aS,11aR)-11a-ethyl-N-(2-methylpyridin-3-yl)-9-oxo-6,7,7a,8,9,10,11,11a-octahydrodibenzo[b,d]oxepine-3-carboxamide; compound with (7aR,11aS)-11a-ethyl-N-(2-methylpyridin-3-yl)-9-oxo-6,7,7a,8,9,10,11,11a-octahydrodibenzo[b,d]oxepine-3-carboxamide (150, R⁶=2-Methylpyridin-3-yl, R⁸=H, R⁹=H) (2.20 g, 96%). LC/MS, method 3, Rₜ=1.91 min, MS m/z: 379 (M+H)⁺; ¹H NMR (400 MHz, CDCl₃) δ 8.40-8.32 (m, 2H), 7.67 (s, 1H), 7.64 (dd, J=8.2, 2.1 Hz, 1H), 7.54 (d, J=2.1 Hz, 1H), 7.42 (d, J=8.2 Hz, 1H), 7.23 (dd, J=8.1, 4.8 Hz, 1H), 4.35-4.29 (m, 1H), 3.84-3.77 (m, 1H), 2.80-2.63 (m, 2H), 2.62 (s, 3H), 2.60-2.47 (m, 2H), 2.46-2.24 (m, 3H), 2.14-2.06 (m, 1H), 1.86-1.78 (m, 1H), 1.63-1.51 (m, 2H), 0.69 (t, J=7.2 Hz, 3H).

### Step #16: (2′R,7aS,11aR)-11a-Ethyl-N-(2-methylpyridin-3-yl)-7,7a,8,10,11,11a-hexahydro-6H-Spiro[dibenzo[b,d]oxepine-9,2′-oxirane]-3-carb oxamide; compound with (2′S,7aR,11aS)-11a-ethyl-N-(2-methylpyridin-3-yl)-7,7a,8,10,11,11a-hexahydro-6H-spiro[dibenzo[b,d]oxepine-9,2′-oxirane]-3-carboxamide (151, R⁶=2-Methylpyridin-3-yl, R⁸=H, R⁹=H)

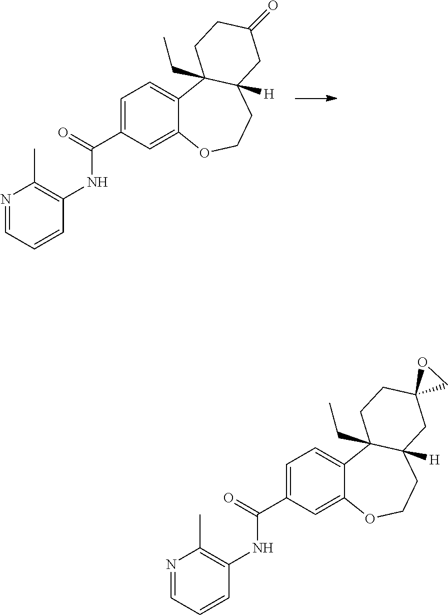

A round bottom flask equipped with stir bar and nitrogen line was charged with sodium hydride (60 wt % dispersion in mineral oil, 0.106 g, 2.64 mmol) and DMSO (6 mL). The mixture was heated at about 60° C. for about 1 h. The mixture was cooled to rt then diluted with THF (6 mL). The mixture was cooled to about 0° C. then trimethylsulfoxonium iodide (0.581 g, 2.64 mmol) was added. The mixture was stirred for about 10 min then (7aS,11aR)-11a-ethyl-N-(2-methylpyridin-3-yl)-9-oxo-6,7,7a,8,9,10,11,11a-octahydrodibenzo[b,d]oxepine-3-carboxamide; compound with (7aR,11aS)-11a-ethyl-N-(2-methylpyridin-3-yl)-9-oxo-6,7,7a,8,9,10,11,11a-octahydrodibenzo[b,d]oxepine-3-carboxamide (150, R⁶=2-Methylpyridin-3-yl, R⁸=H, R⁹=H) (0.500 g, 1.32 mmol) in THF (6 mL) was added over about 10 min. The mixture was stirred in the ice bath for about 5 min then the bath was removed and the mixture was allowed to warm to rt for about 18 h. The mixture was concentrated under reduced pressure and partitioned between EtOAc (75 mL) and water (75 mL). The layers were separated and the organic solution was washed with water (3×50 mL). The organic solution was dried over MgSO₄, filtered and concentrated under reduced pressure. The material was purified on silica gel (12 g) using a gradient of 50-100% EtOAc in heptane. Fractions containing product were combined and concentrated, then dried to constant weight at about 60° C. under reduced pressure to give (2′R,7aS,11aR)-11a-ethyl-N-(2-methylpyridin-3-yl)-7,7a,8,10,11,11a-hexahydro-6H-spiro[dibenzo[b,d]oxepine-9,2′-oxirane]-3-carboxamide; compound with (2′S,7aR,11aS)-11a-ethyl-N-(2-methylpyridin-3-yl)-7,7a,8,10,11,11a-hexahydro-6H-spiro[dibenzo[b,d]oxepine-9,2′-oxirane]-3-carboxamide (151, R⁶=2-Methylpyridin-3-yl, R⁸=H, R⁹=H) (0.450 g, 87%) as a white solid. LC/MS, method 3, Rₜ=2.03 min, MS m/z: 393 (M+H)⁺; ¹H NMR (400 MHz, CDCl₃) δ 8.38 (dd, J=8.2, 1.3 Hz, 1H), 8.33 (dd, J=4.8, 1.5 Hz, 1H), 7.64 (s, 1H), 7.59 (dd, J=8.2, 2.1 Hz, 1H), 7.48 (d, J=2.0 Hz, 1H), 7.39 (d, J=8.3 Hz, 1H), 7.22 (dd, J=8.1, 4.8 Hz, 1H), 4.30-4.25 (m, 1H), 3.79-3.72 (m, 1H), 2.78-2.69 (m, 1H), 2.67 (d, J=4.5 Hz, 1H), 2.63 (d, J=4.5 Hz, 1H), 2.61 (s, 3H), 2.50-2.40 (m, 1H), 2.40-2.18 (m, 3H), 2.05-1.98 (m, 1H), 1.86-1.78 (m 1H), 1.68-1.48 (m, 2H), 1.39-1.30 (m, 1H), 0.94-0.86 (m, 1H), 0.68 (t, J=7.6 Hz, 3H).

### Step #17: (7aS,9R,11aR)-11a-Ethyl-9-hydroxy-N-(2-methylpyridin-3-yl)-9-propyl-6,7,7a,8,9,10,11,11a-octahydrodibenzo[b,d]oxepine-3-carboxamide; compound with (7aR,9S,11aS)-11a-ethyl-9-hydroxy-N-(2-methylpyridin-3-yl)-9-propyl-6,7,7a,8,9,10,11,11a-octahydrodibenzo[b,d]oxepine-3-carboxamide (152, R⁵=Ethyl, R⁶=2-Methylpyridin-3-yl, R⁸=H, R⁹=H)

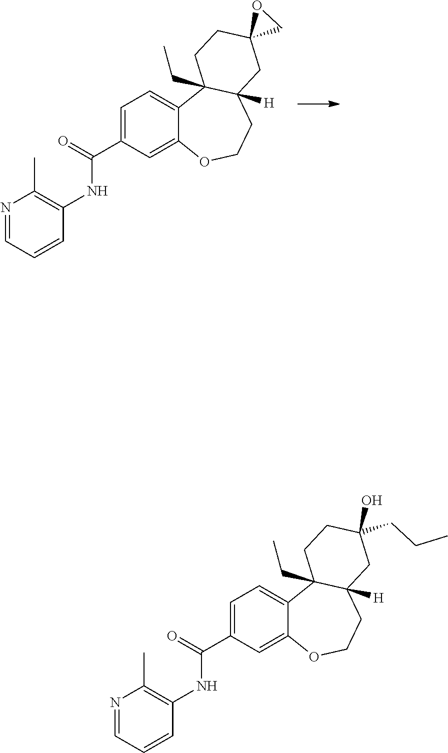

A 3 necked round bottom flask with stir bar, nitrogen line, septum and thermometer was charged with (2′R,7aS,11aR)-11a-ethyl-N-(2-methylpyridin-3-yl)-7,7a,8,10,11,11a-hexahydro-6H-spiro[dibenzo[b,d]oxepine-9,2′-oxirane]-3-carb oxamide; compound with (2′S,7aR,11aS)-11a-ethyl-N-(2-methylpyridin-3-yl)-7,7a,8,10,11,11a-hexahydro-6H-spiro[dibenzo[b,d]oxepine-9,2′-oxirane]-3-carboxamide (151, R⁶=2-Methylpyridin-3-yl, R⁸=H, R⁹=H) (0.140 g, 0.357 mmol), THF (6 mL) and copper(I)iodide (0.009 g, 0.05 mmol). The mixture was cooled to about 0° C. then ethylmagnesium bromide (3 M in Et₂O, 0.71 mL, 2.14 mmol) was added dropwise. After about 5 min the reaction was quenched with saturated aqueous NH₄Cl (10 mL). Water (10 mL) was added and the mixture was extracted with EtOAc (25 mL then 10 mL). The combined organics were washed with saturated aqueous NaCl (10 mL) then dried over MgSO₄, filtered and concentrated under reduced pressure. The material was purified on silica gel (12 g) using a gradient of 50-100% EtOAc in heptane. Fractions containing product were combined and concentrated under reduced pressure. The material was triturated with heptane (˜15 mL) and the white solid was collected by filtration and washed with heptane (2 mL). The material was dried under reduced pressure at about 60° C. for about 16 h to give (7aS,9R,11aR)-11a-ethyl-9-hydroxy-N-(2-methylpyridin-3-yl)-9-propyl-6,7,7a,8,9,10,11,11a-octahydrodibenzo[b,d]oxepine-3-carboxamide; compound with (7aR,9S,11aS)-11a-ethyl-9-hydroxy-N-(2-methylpyridin-3-yl)-9-propyl-6,7,7a,8,9,10,11,11a-octahydrodibenzo[b,d]oxepine-3-carboxamide (152, R⁵=Ethyl, R⁶=2-Methylpyridin-3-yl, R⁸=H, R⁹=H) (0.110 g, 73%) LC/MS, method 2, Rₜ=2.11 min, MS m/z: 423 (M+H)⁺; ¹H NMR (400 MHz, DMSO-d₆) δ 9.97 (s, 1H), 8.33 (dd, J=4.8, 1.6 Hz, 1H), 7.71 (dd, J=8.0, 1.6 Hz, 1H), 7.64 (dd, J=8.2, 2.0 Hz, 1H), 7.53 (d, J=2.0 Hz, 1H), 7.37 (d, J=8.4 Hz, 1H), 7.27 (dd, J=7.9, 4.7 Hz, 1H), 4.25-4.15 (m, 1H), 3.98 (s, 1H), 3.71-3.65 (m, 1H), 2.61-2.52 (m, 1H), 2.43 (s, 3H), 2.37-2.28 (m, 1H), 2.21-2.01 (m, 2H), 1.79-1.64 (m, 1H), 1.60-1.37 (m, 4H), 1.33-1.10 (m, 6H), 0.79 (t, J=7.0 Hz, 3H), 0.57 (t, J=7.4 Hz, 3H).

Additional examples, prepared in a manner similar to the preparation of Example #119 are listed in Table 1.

Chiral separation of Example 119 (152, R⁵=Ethyl, R⁶=2-Methylpyridin-3-yl, R⁸=H, R⁹=H) The separation of enantiomers was accomplished using chiral separation method 10. The first peak eluted was (7aR,9S,11aS)-11a-ethyl-9-hydroxy-N-(2-methylpyridin-3-yl)-9-propyl-6,7,7a,8,9,10,11,11a-octahydrodibenzo[b,d]oxepine-3-carboxamide (A-1388162.0) (Example 120) and the second was (7aS,9R,11aR)-11a-ethyl-9-hydroxy-N-(2-methylpyridin-3-yl)-9-propyl-6,7,7a,8,9,10,11,11a-octahydrodibenzo[b,d]oxepine-3-carboxamide (A-1388163.0) (Example 121). NMR and LCMS data for single isomers was essentially identical to the racemic mixture.

Additional examples, prepared in a manner similar to the preparation of Examples #120 and #121, are listed in Table 2

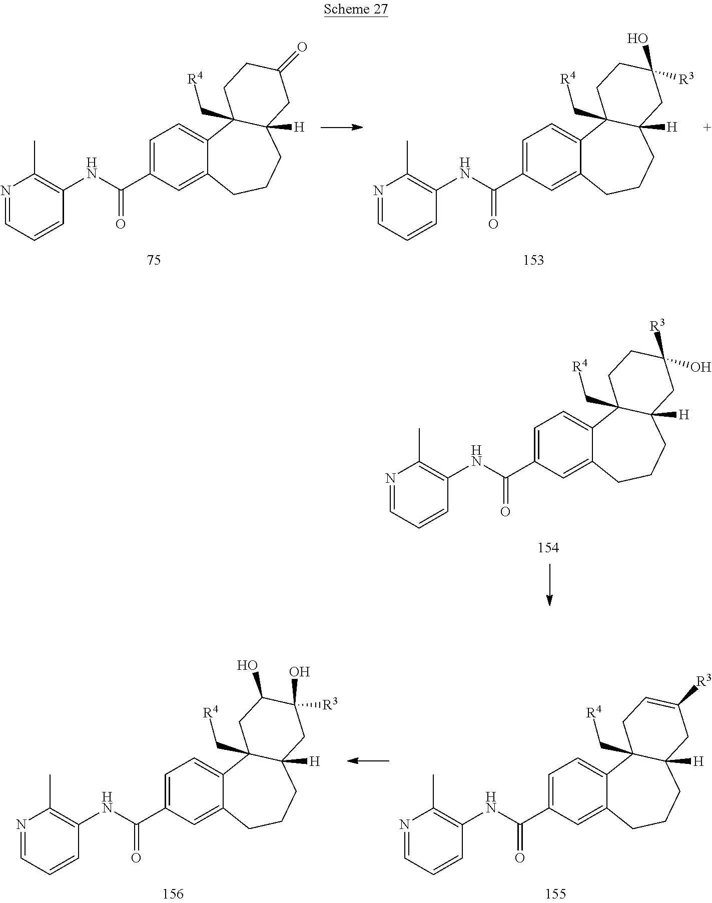

### Example 122

### (7aS,9R,11aR)-11a-Ethyl-9-hydroxy-9-phenyl-6,7,7a,8,9,10,11,11a-octahydro-5H-dibenzo[a,c]cycloheptene-3-carboxylic acid (2-methyl-pyridin-3-yl)-amide (153, R³=Phenyl, R⁴=Methyl) and Example 123: (7aR,9S,11aS)-11a-ethyl-9-hydroxy-9-phenyl-6,7,7a,8,9,10,11,11a-octahydro-5H-dibenzo[a,c]cycloheptene-3-carboxylic acid (2-methyl-pyridin-3-yl)-amide (153, R³=Phenyl, R⁴=Methyl)

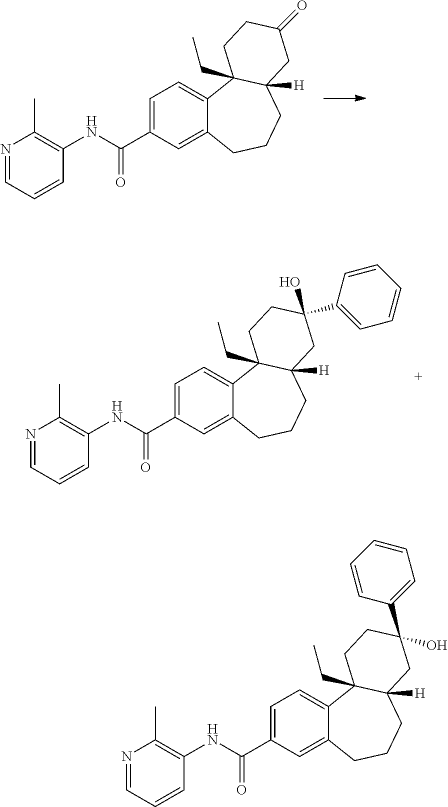

A solution of (7aR,11aS)-11a-ethyl-9-oxo-6,7,7a,8,9,10,11,11a-octahydro-5H-dibenzo[a,c]cycloheptene-3-carboxylic acid (2-methyl-pyridin-3-yl)-amide; compound with (7aS,11aR)-11a-ethyl-9-oxo-6,7,7a,8,9,10,11,11a-octahydro-5H-dibenzo[a,c]cycloheptene-3-carboxylic acid (2-methyl-pyridin-3-yl)-amide (75, R⁴=Methyl) (1.60 g, 2.97 mmol) in THF (64 mL) was cooled to about 0° C. under nitrogen. Phenylmagnesium bromide (14.9 mL, 14.9 mmol, 1M solution in THF) was added dropwise maintaining reaction temperature below about 7° C. The mixture was stirred at about 0° C. for about 1 h, and then quenched by addition of saturated aqueous NH₄Cl (25 mL). The reaction was diluted with EtOAc (100 mL) and washed with saturated aqueous NH₄Cl (3×25 mL). The organic layer was dried over Na₂SO₄, filtered and concentrated under reduced pressure. The residue was purified on silica gel (80 g) using EtOAc as eluant. Fractions containing the second peak (major component) were combined and concentrated under reduced pressure to yield (7aS,9S,11aR)-11a-ethyl-9-hydroxy-9-phenyl-6,7,7a,8,9,10,11,11a-octahydro-5H-dibenzo[a,c]cycloheptene-3-carboxylic acid (2-methyl-pyridin-3-yl)-amide; compound with (7aR,9R,11aS)-11a-ethyl-9-hydroxy-9-phenyl-6,7,7a,8,9,10,11,11a-octahydro-5H-dibenzo[a,c]cycloheptene-3-carboxylic acid (2-methyl-pyridin-3-yl)-amide (153, R³=Phenyl, R⁴=Methyl) (0.823 g, 63%). LC/MS, method 4, Rₜ=1.59 min, MS m/z 455 (M+H)⁺. ¹H NMR (400 MHz, DMSO-d₆) δ 9.96 (s, 1H), 8.32 (dd, J=4.8, 1.6 Hz, 1H), 7.85-7.78 (m, 1H), 7.77-7.71 (m, 2H), 7.55-7.46 (m, 3H), 7.39-7.32 (m, 2H), 7.30-7.21 (m, 2H), 4.76 (s, 1H), 3.08-2.95 (m, 1H), 2.95-2.82 (m, 1H), 2.46 (s, 3H), 2.46-2.34 (m, 2H), 2.14-2.00 (m, 2H), 1.96-1.80 (m, 2H), 1.80-1.68 (m, 1H), 1.64-1.45 (m, 4H), 1.41-1.17 (m, 2H), 0.55 (t, J=7.3 Hz, 3H). Fractions containing the first peak (minor component) were combined and concentrated under reduced pressure to yield (7aS,9R,11aR)-11a-ethyl-9-hydroxy-9-phenyl-6,7,7a,8,9,10,11,11a-octahydro-5H-dibenzo[a,c]cycloheptene-3-carboxylic acid (2-methyl-pyridin-3-yl)-amide; compound with (7aR,9S,11aS)-11a-ethyl-9-hydroxy-9-phenyl-6,7,7a,8,9,10,11,11a-octahydro-5H-dibenzo[a,c]cycloheptene-3-carboxylic acid (2-methyl-pyridin-3-yl)-amide (153, R³=Phenyl, R⁴=Methyl) (0.125 g, 9%) as a white solid. LC/MS, method 2, Rₜ=2.29 min, MS m/z 455 (M+H)⁺. ¹H NMR (400 MHz, DMSO) δ 9.96 (s, 1H), 8.32 (dd, J=4.8, 1.6 Hz, 1H), 7.80-7.70 (m, 3H), 7.47 (d, J=8.4 Hz, 1H), 7.28-7.16 (m, 5H), 7.16-7.07 (m, 1H), 4.85 (s, 1H), 3.09-2.97 (m, 1H), 2.95-2.85 (m, 1H), 2.56-2.48 (m, 1H), 2.44 (s, 3H), 2.44-2.36 (m, 1H), 2.34-2.21 (m, 1H), 2.16-2.05 (m, 1H), 2.03-1.91 (m, 1H), 1.91-1.79 (m, 1H), 1.77-1.66 (m, 2H), 1.63-1.41 (m, 4H), 1.34-1.24 (m, 1H), 0.65 (t, J=7.4 Hz, 3H).

The minor product was further purified using chiral chromatography method 12 to yield first (7aS,9R,11aR)-11a-ethyl-9-hydroxy-9-phenyl-6, 7,7a,8,9,10,11,11a-octahydro-5H-dibenzo[a,c]cycloheptene-3-carboxylic acid (2-methyl-pyridin-3-yl)-amide (153, R³=Phenyl, R⁴=Methyl); and second (7aR,9S,11aS)-11a-ethyl-9-hydroxy-9-phenyl-6,7,7a,8,9,10,11,11a-octahydro-5H-dibenzo[a,c]cycloheptene-3-carboxylic acid (2-methyl-pyridin-3-yl)-amide (153, R³=Phenyl, R⁴=Methyl) NMR and LCMS data for single isomers was essentially identical to the racemic mixture.

### Example 124

### (7aS,9R,10R,11aR)-11a-Ethyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide (156, R³=Phenyl, R⁴=Methyl) and Example 125: (7aR,9S,10S,11aS)-11a-ethyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide (156, R³=Phenyl, R⁴=Methyl)

### Step #1: (7aS,11aR)-11a-Ethyl-9-phenyl-6,7,7a,8,11,11a-hexahydro-5H-dibenzo[a,c]cycloheptene-3-carboxylic acid (2-methyl-pyridin-3-yl)-amide; compound with (7aR,11aS)-11a-ethyl-9-phenyl-6,7,7a,8,11,11a-hexahydro-5H-dibenzo[a,c]cycloheptene-3-carboxylic acid (2-methyl-pyridin-3-yl)-amide (155, R³=Phenyl, R⁴=Methyl)

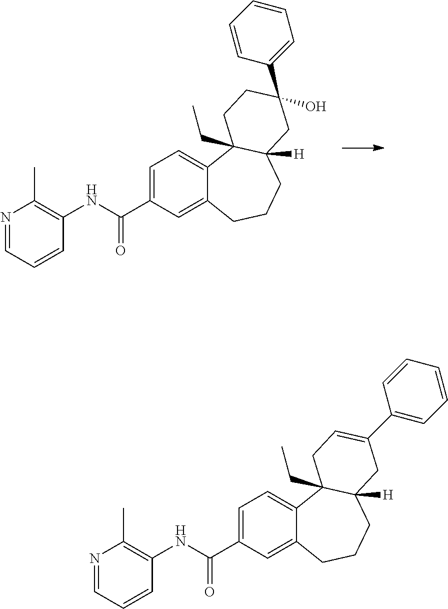

A suspension of (7aS,9S,11aR)-11a-Ethyl-9-hydroxy-9-phenyl-6,7,7a,8,9,10,11,11a-octahydro-5H-dibenzo[a,c]cycloheptene-3-carboxylic acid (2-methyl-pyridin-3-yl)-amide; compound with (7aR,9R,11aS)-11a-ethyl-9-hydroxy-9-phenyl-6,7,7a,8,9,10,11,11a-octahydro-5H-dibenzo[a,c]cycloheptene-3-carboxylic acid (2-methyl-pyridin-3-yl)-amide (154, R³=Phenyl, R⁴=Methyl) (820 mg, 1.80 mmol) and pTsOH (721 mg, 3.79 mmol) in toluene (40 mL) was heated at reflux, removing water into a Dean-Stark trap for about 90 min. The reaction was cooled to rt and washed with saturated aqueous NaHCO₃ (2×25 mL). The organic layer was dried over Na₂SO₄, filtered and concentrated under reduced pressure. The residue was purified on silica gel (40 g) using a gradient of 50-100% ethyl acetate in heptane. Fractions containing product were combined and concentrated to yield (7aS,11aR)-11a-ethyl-9-phenyl-6,7,7a,8,11,11a-hexahydro-5H-dibenzo[a,c]cycloheptene-3-carboxylic acid (2-methyl-pyridin-3-yl)-amide; compound with (7aR,11aS)-11a-ethyl-9-phenyl-6,7,7a,8,11,11a-hexahydro-5H-dibenzo[a,c]cycloheptene-3-carboxylic acid (2-methyl-pyridin-3-yl)-amide (155, R³=Phenyl, R⁴=Methyl) (515 mg, 65%) as an off-white solid. LC/MS, method 4, Rₜ=2.44 min, MS m/z 437 (M+H)⁺. ¹H NMR (400 MHz, DMSO-d₆) δ 9.88 (s, 1H), 8.29 (dd, J=4.7, 1.5 Hz, 1H), 7.73-7.64 (m, 2H), 7.61 (dd, J=8.2, 1.9 Hz, 1H), 7.33-7.27 (m, 2H), 7.27-7.11 (m, 5H), 6.38-6.33 (m, 1H), 3.28-3.18 (m, 1H), 3.09-2.99 (m, 1H), 2.90-2.81 (m, 1H), 2.46-2.41 (m, 1H), 2.38 (s, 3H), 2.37-2.29 (m, 1H), 2.27-2.10 (m, 3H), 2.02-1.88 (m, 1H), 1.75-1.61 (m, 3H), 1.54-1.43 (m, 1H), 0.61 (t, J=7.3 Hz, 3H).

### Step #2: (7aS,9R,10R,11aR)-11a-Ethyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide (156, R³=Phenyl, R⁴=Methyl) and (7aR,9S,10S,11aS)-11a-ethyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide (156, R³=Phenyl, R⁴=Methyl)

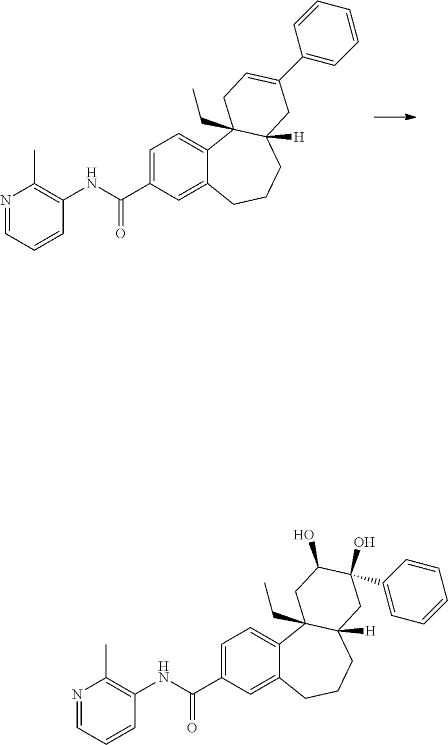

A solution of (7aS,11aR)-11a-Ethyl-9-phenyl-6,7,7a,8,11,11a-hexahydro-5H-dibenzo[a,c]cycloheptene-3-carboxylic acid (2-methyl-pyridin-3-yl)-amide; compound with (7aR,11aS)-11a-ethyl-9-phenyl-6,7,7a,8,11,11a-hexahydro-5H-dibenzo[a,c]cycloheptene-3-carboxylic acid (2-methyl-pyridin-3-yl)-amide (155, R³=Phenyl, R⁴=Methyl) (150 mg, 0.344 mmol) in THF (18 mL) and water (3 mL) was treated with NMO (80 mg, 0.69 mmol) and osmium(VIII) oxide (873 mg, 0.086 mmol) and the mixture was allowed to stir at rt for about 72 h. The reaction was diluted with water (45 mL) and extracted with EtOAc (2×20 mL). The combined organic layers were dried over Na₂SO₄, filtered and concentrated under reduced pressure. The residue was purified on silica gel (4 g) using EtOAc as eluant to yield (7aS,9R,10R,11aR)-11a-ethyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide compound with (7aR,9S,10S,11aS)-11a-ethyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide (156, R³=Phenyl, R⁴=Methyl) (124 mg, 76%) as an off-white solid. LC/MS, method 2, Rₜ=2.06 min, MS m/z 471 (M+H)⁺. ¹H NMR (400 MHz, DMSO-d₆) δ 9.97 (s, 1H), 8.38-8.29 (m, 1H), 7.83 (dd, J=8.2, 1.9 Hz, 1H), 7.78-7.71 (m, 2H), 7.57 (d, J=8.5 Hz, 1H), 7.27 (dd, J=7.9, 4.8 Hz, 1H), 7.24-7.14 (m, 4H), 7.13-7.07 (t, J=6.9 Hz, 1H), 4.56 (s, 1H), 4.44 (d, J=6.2 Hz, 1H), 4.11-4.03 (m, 1H), 3.07-3.95 (m, 1H), 2.94-2.85 (m, 1H), 2.50-2.42 (m, 5H), 2.27-2.15 (m, 1H), 2.12-2.00 (m, 1H), 1.84-1.33 (m, 6H), 1.32-1.23 (m, 1H), 0.66 (t, J=7.4 Hz, 3H).

The racemic product was further purified using chiral chromatography method 13 to yield first (7aR,9S,10S,11aS)-11a-ethyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide (156, R³=Phenyl, R⁴=Methyl) and second (7aS,9R,10R,11aR)-11a-Ethyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide (156, R³=Phenyl, R⁴=Methyl) NMR and LCMS data for single isomers was essentially identical to the racemic mixture.

### Examples #126

### (7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2-amino-phenyl)-amide (85, R⁴=Phenyl, R⁵=Methyl, R⁶=2-Amino-phenyl) and Example 127: (3R,4aS,11bS)-9-(1H-benzoimidazol-2-yl)-11b-benzyl-3-ethyl-2,3,4,4a,5,6,7,11b-octahydro-1H-dibenzo[a,c]cyclohepten-3-ol

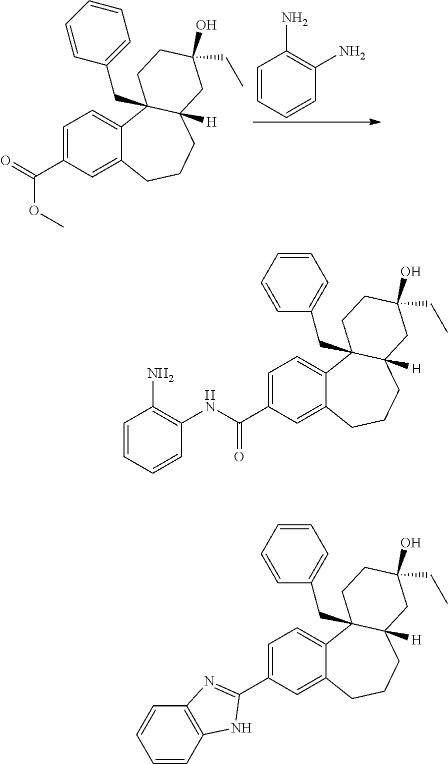

To a solution of benzene-1,2-diamine (0.048 g, 0.446 mmol) in toluene (1 mL) was added a solution of trimethylaluminum (2.0 M in toluene) (0.38 mL, 0.76 mmol) and the mixture was stirred for about 15 min at rt. A solution of (7aS,9R,11aS)-11a-benzyl-9-ethyl-9-hydroxy-6,7,7a,8,9,10,11,11a-octahydro-5H-dibenzo[a,c]cycloheptene-3-carboxylic acid methyl ester (80, R⁴=Phenyl, R⁵=Methyl) (0.100 g, 0.255 mmol) in toluene (1.5 mL) was added and the reaction mixture was heated to about 100° C. for about 3 days. The mixture was cooled to rt and then water (10 mL) and EtOAc (10 mL) were added and the layers were separated. The aqueous phase was extracted with EtOAc (2×10 mL). The combined organics were dried over MgSO₄, filtered, and concentrated under reduced pressure. The crude material was purified on silica gel (25 g) eluting with a gradient of 0-10% MeOH in DCM. The residue was further purified on silica gel (25 g) eluting with 0-8% MeOH in DCM. The early eluting product fractions were collected, concentrated and then triturated with 1:9 MeOH/water (2 mL). The solids collected were rinsed with excess water and then dried in a 70° C. vacuum oven to furnish (7aS,9R,11aS)-11a-benzyl-9-ethyl-9-hydroxy-6,7,7a,8,9,10,11,11a-octahydro-5H-dibenzo[a,c]cycloheptene-3-carboxylic acid (2-amino phenyl)-amide (85, R⁴=Phenyl, R⁵=Methyl, R⁶=2-Amino phenyl) (0.030 g, 25%); LC/MS method 2, Rₜ=2.49 min, MS m/z 469 (M+H)⁺; ¹H NMR (400 MHz, DMSO-d₆) δ 9.58 (s, 1H), 7.81 (d, J=2.1 Hz, 1H), 7.55 (dd, J=8.2, 2.1 Hz, 1H), 7.15 (dd, J=7.8, 1.5 Hz, 1H), 7.14-7.01 (m, 3H), 7.00-6.92 (m, 1H), 6.78 (dd, J=8.0, 1.4 Hz, 2H), 6.67-6.53 (m, 3H), 4.87 (bs, 2H), 3.88 (s, 1H), 3.58 (d, J=12.9 Hz, 1H), 3.29-3.22 (m, 1H), 3.07-2.97 (m, 1H), 2.58 (d, J=1.1 Hz, 1H), 2.03-1.71 (m, 3H), 1.71-1.03 (m, 10H), 0.71 (t, J=7.4 Hz, 3H). The later eluting product fractions were collected, concentrated and then triturated with about 2 mL of 1:9 MeOH/water. The solids collected were then triturated with 8:2 heptane/EtOAc (2×2 mL). The filtrates were concentrated and dried in a 70° C. vacuum oven to provide (3R,4aS,11bS)-9-(1H-benzoimidazol-2-yl)-11b-benzyl-3-ethyl-2,3,4,4a,5,6,7,11b-octahydro-1H-dibenzo[a,c]cyclohepten-3-ol (0.012 g, 11%); LC/MS method 2, Rₜ=2.54 min, MS m/z 451 (M+H)⁺, ¹H NMR (400 MHz, DMSO-d₆) δ 12.78 (s, 1H), 8.00 (d, J=2.1 Hz, 1H), 7.72-7.61 (m, 2H), 7.55-7.47 (m, 1H), 7.23-7.13 (m, 2H), 7.10-6.96 (m, 3H), 6.81 (d, J=8.5 Hz, 1H), 6.62-6.55 (m, 2H), 3.89 (s, 1H), 3.60 (d, J=12.9 Hz, 1H), 3.09-3.00 (m, 1H), 2.59 (d, J=13.0 Hz, 1H), 1.94-1.70 (m, 3H), 1.69-1.51 (m, 2H), 1.49-1.05 (m, 9H), 0.70 (t, J=7.4 Hz, 3H).

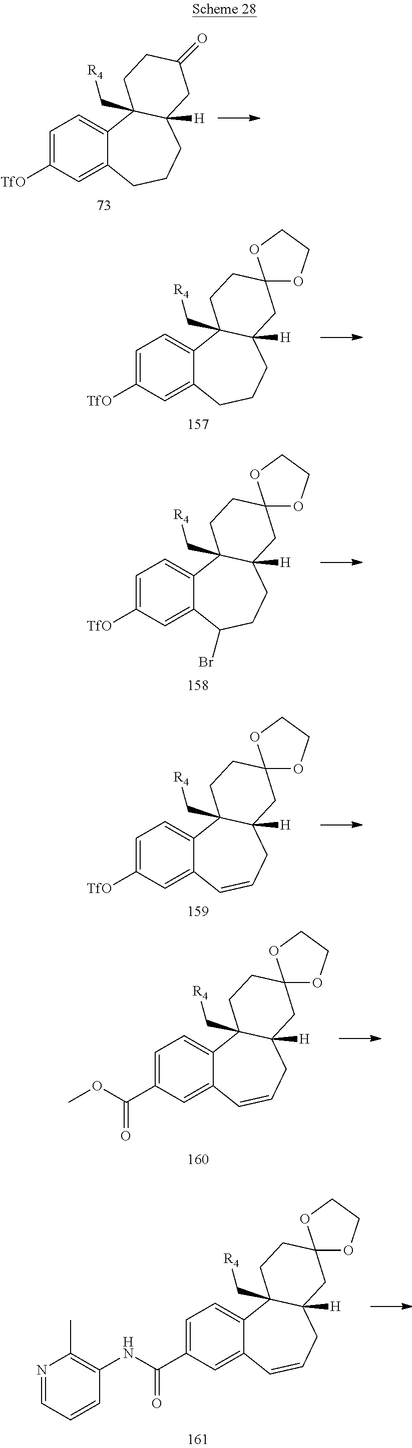

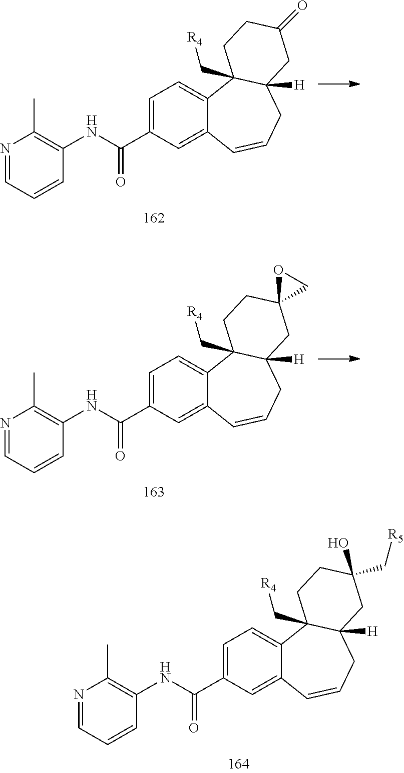

### Example 128

### (7aS,9R,11aR)-11a-Ethyl-9-hydroxy-9-propyl-7a,8,9,10,11,11a-hexahydro-7H-dibenzo[a,c]cycloheptene-3-carboxylic acid (2-methyl-pyridin-3-yl)-amide compound with (7aR,9S,11aS)-11a-ethyl-9-hydroxy-9-propyl-7a,8,9,10,11,11a-hexahydro-7H-dibenzo[a,c]cycloheptene-3-carboxylic acid (2-methyl-pyridin-3-yl)-amide (164, R⁴=Methyl, R⁵=Ethyl)

### Step #1: (+/−) Compound 157 (R⁴=Methyl)

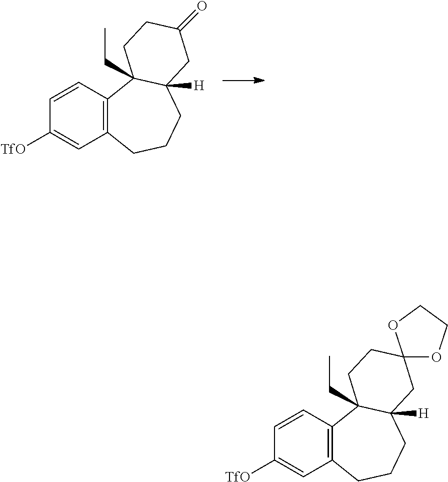

A solution of trifluoro-methanesulfonic acid (7aR,11aS)-11a-ethyl-9-oxo-6,7,7a,8,9,10,11,11a-octahydro-5H-dibenzo[a,c]cyclohepten-3-yl ester; compound with trifluoro-methanesulfonic acid (7aS,11aR)-11a-ethyl-9-oxo-6,7,7a,8,9,10,11,11a-octahydro-5H-dibenzo[a,c]cyclohepten-3-yl ester (73, R⁴=Methyl) (0.800 g, 2.05 mmol) and p-toluenesulfonic acid monohydrate (0.039 g, 0.20 mmol) in toluene (20.5 mL) was treated with ethylene glycol (0.57 mL, 10 mmol), and the reaction mixture was heated at reflux for about 2 h. After cooling to rt, the reaction mixture was partitioned between EtOAc (100 mL) and saturated aqueous NaHCO₃ (100 mL). After separating the layers, the organic phase was washed with saturated aqueous NaCl (100 mL), dried over Na₂SO₄, filtered, and concentrated under reduced pressure to give (+/−) Compound 157 (R⁴=Methyl) (0.900 g, 100%), which was used directly without further purification. LC/MS, method 3, Rₜ=3.04 min, MS m/z 435 (M+H)⁺. ¹H NMR (400 MHz, CDCl₃) δ 7.29-7.24 (m, 1H), 7.05-6.97 (m, 2H), 4.00-3.85 (m, 4H), 3.06-2.94 (m, 1H), 2.81-2.72 (m, 1H), 2.44-2.36 (m, 1H), 2.31-2.12 (m, 2H), 2.12-2.01 (m, 1H), 1.89-1.40 (m, 8H), 1.39-1.29 (m, 1H), 0.69-0.60 (t, J=7.4 Hz, 3H).

### Step #2: (+/−) Compound 158 (R⁴=Methyl)

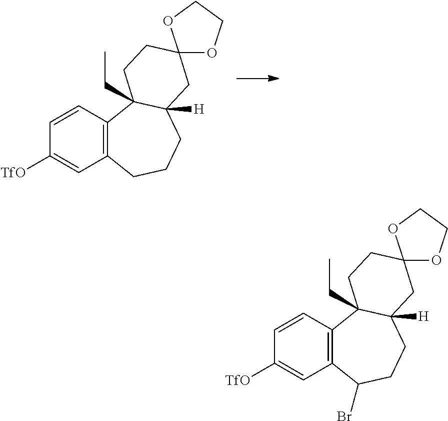

A mixture of (+/−) Compound 157 (R⁴=Methyl) (0.89 g, 2.0 mmol), N-bromosuccinimide (0.438 g, 2.46 mmol), and 2,2′-azobis(2-methylpropionitrile) (0.034 g, 0.20 mmol) in CCl₄ (20.5 mL) was heated at about reflux for about 1 h. After cooling to rt, the reaction mixture was partitioned between DCM (50 mL) and saturated aqueous NaHCO₃ (50 mL). After separating the layers, the organic phase was washed with saturated aqueous NaCl (50 mL), dried over Na₂SO₄, filtered, and concentrated under reduced pressure. The residue was purified on silica gel (40 g) using a gradient of 0-25% EtOAc in heptane. Collection and concentration of product containing fractions gave (+/−) Compound 158 (R⁴=Methyl) (0.593 g, 56%). LC/MS, method 3, Rₜ=3.01 min, MS m/z 513/515 (M+H)⁺. ¹H NMR (400 MHz, CDCl₃) δ 7.35 (d, J=8.9 Hz, 1H), 7.21-7.13 (m, 2H), 5.61 (t, J=3.5 Hz, 1H), 3.99-3.85 (m, 4H), 2.81-2.68 (m, 1H), 2.52-2.38 (m, 2H), 2.32-2.10 (m, 3H), 1.71-1.37 (m, 7H), 0.66 (t, J=7.4 Hz, 3H).

### Step #3: (+/−) Compound 159 (R⁴=Methyl)

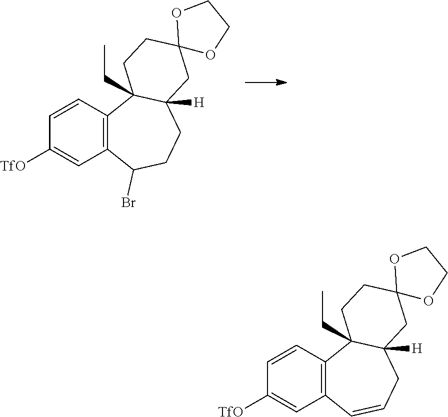

A solution of (+/−) Compound 158 (R⁴=Methyl) (0.59 g, 1.2 mmol) in MeCN (11.5 mL) was treated with TEA (0.18 mL, 1.3 mmol) and the reaction mixture was heated at about 80° C. for about 16 h. The reaction was allowed to cool to rt and then concentrated under reduced pressure. The residue was partitioned between water (50 mL) and EtOAc (50 mL). After separating the layers, the organic phase was washed with saturated aqueous NaCl (50 mL), dried over Na₂SO₄, filtered, and concentrated under reduced pressure. The residue was purified on silica gel (25 g) using a gradient of 0 to 30% EtOAc in heptane. Collection and concentration of the appropriate fractions gave (+/−) Compound 159 (R⁴=Methyl) (0.363 g, 73%). LC/MS, method 3, Rₜ=2.99 min, MS m/z 433 (M+H)⁺.

### Step #4: (+/−) Compound 160 (R⁴=Methyl)

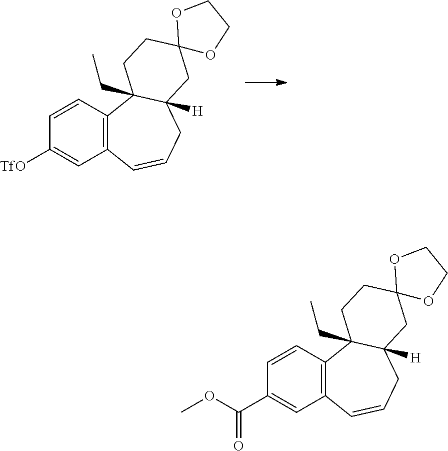

A mixture of (+/−) Compound 159 (R⁴=Methyl) (0.363 g, 0.839 mmol), Pd₂(dba)₃ (0.023 g, 0.025 mmol), and Xantphos (0.049 g, 0.084 mmol) in DMF (8.4 mL) was degassed under vacuum for about 20 min. An atmosphere of carbon monoxide from a balloon was used to break the vacuum, and this cycle was repeated two more times before the reaction was left to stir under an atmosphere of carbon monoxide. TEA (0.47 mL, 3.4 mmol) and MeOH (0.41 mL, 10 mmol) were added, and the reaction mixture was heated at about 100° C. for about 16 h. After cooling to rt, the reaction mixture was concentrated under reduced pressure and the residue adsorbed onto silica gel (1.5 g). The residue was purified on silica gel (12 g) using a gradient of 0-25% EtOAc in heptane. Collection and concentration of the appropriate fractions gave (+/−) Compound 160 (R⁴=Methyl) (0.171 g, 60% yield). LC/MS, method 3, Rₜ=2.73 min, MS m/z 343 (M+H)⁺. ¹H NMR (400 MHz, CDCl₃) δ 7.87 (d, J=2.0 Hz, 1H), 7.80 (dd, J=8.3, 2.0 Hz, 1H), 7.39 (d, J=8.3 Hz, 1H), 6.39 (dd, J=12.2, 3.1 Hz, 1H), 5.85-5.75 (m, 1H), 3.97-3.80 (m, 7H), 2.90-2.78 (m, 1H), 2.52-2.44 (m, 1H), 2.27-2.13 (m, 2H), 1.75-1.32 (m, 7H), 0.75 (t, J=7.5 Hz, 3H).

### Step #5: (+/−) Compound 161 (R⁴=Methyl)

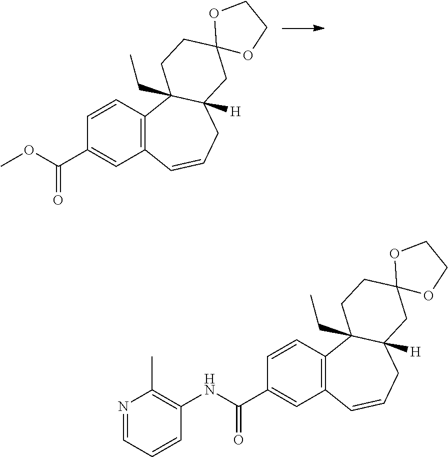

A suspension of (+/−) Compound 160 (R⁴=Methyl) (0.171 g, 0.499 mmol) and 3-amino-2-picoline (0.095 g, 0.87 mmol) in toluene (5.0 mL) at rt was treated with LiHMDS (1.50 mL, 1.50 mmol, 1 M solution in THF) and the resulting suspension was stirred at rt for about 5 min. The reaction mixture was quenched at rt by addition of saturated aqueous NH₄Cl (15 mL). The mixture was diluted with EtOAc (10 mL), and after separating the layers, the organic phase was washed with saturated aqueous NaCl (15 mL), dried over Na₂SO₄, filtered, and concentrated under reduced pressure. The residue was purified on silica gel (12 g) using a gradient of 0-25% EtOAc in heptane. Collection and concentration of the appropriate fractions gave (+/−) Compound 161 (R⁴=Methyl) (0.214 g, 100% yield). LC/MS, method 3, Rₜ=2.22 min, MS m/z 419 (M+H)⁺. ¹H NMR (400 MHz, CDCl₃) δ 8.46-8.40 (m, 1H), 8.34 (dd, J=4.8, 1.6 Hz, 1H), 7.73-7.60 (m, 3H), 7.47 (d, J=8.3 Hz, 1H), 7.30-7.19 (m, 1H), 6.42 (dd, J=12.2, 3.0 Hz, 1H), 5.91-5.82 (m, 1H), 3.98-3.82 (m, 4H), 2.93-2.82 (m, 1H), 2.64 (s, 3H), 2.63-2.46 (m, 1H), 2.30-2.14 (m, 2H), 1.63-1.30 (m, 7H), 0.78 (t, J=7.4 Hz, 3H).

### Step #6: (7aS,11aR)-11a-Ethyl-9-oxo-7a,8,9,10,11,11a-hexahydro-7H-dibenzo[a,c]cycloheptene-3-carboxylic acid (2-methyl-pyridin-3-yl)-amide; compound with (7aR,11aS)-11a-ethyl-9-oxo-7a,8,9,10,11,11a-hexahydro-7H-dibenzo[a,c]cycloheptene-3-carboxylic acid (2-methyl-pyridin-3-yl)-amide (162, R⁴=Methyl)

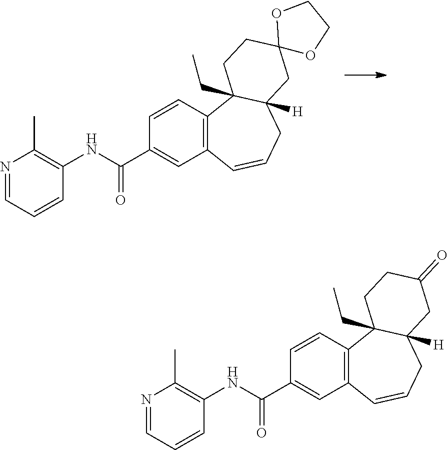

A suspension of (+/−) Compound 161 (R⁴=Methyl) (0.209 g, 0.499 mmol) in DCM (2.2 mL) and water (1.1 mL) was treated with Tfa (0.23 mL, 3.0 mmol) and the mixture was heated at about 40° C. for about 16 h. The reaction mixture was partitioned between DCM (20 mL) and saturated aqueous NaHCO₃ (15 mL). After separating the layers, the organic phase was washed with saturated aqueous NaCl (20 mL), dried over Na₂SO₄, filtered, and concentrated under reduced pressure to give (7aS,11aR)-11a-ethyl-9-oxo-7a,8,9,10,11,11a-hexahydro-7H-dibenzo[a,c]cycloheptene-3-carboxylic acid (2-methyl-pyridin-3-yl)-amide; compound with (7aR,11aS)-11a-ethyl-9-oxo-7a,8,9,10,11,11a-hexahydro-7H-dibenzo[a,c]cycloheptene-3-carboxylic acid (2-methyl-pyridin-3-yl)-amide (162, R⁴=Methyl) (0.171 g, 91%). LC/MS, method 3, Rₜ=1.97 min, MS m/z 375 (M+H)⁺. The sample was used in the next step without further purification.

### Step #7: (+/−) Compound 163 (R⁴=Methyl)

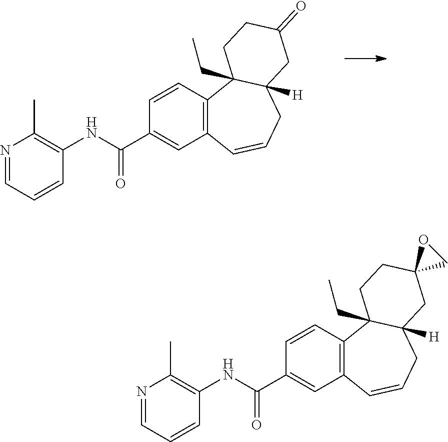

A suspension of NaH (0.032 g, 0.80 mmol, 60% in mineral oil) in DMSO-d₆ (2.0 mL) was heated at about 60° C. for about 20 min, after which it was allowed to cool to rt. Trimethylsulfoxonium iodide (0.176 g, 0.801 mmol) was added in one portion and the reaction mixture was cooled to about 0° C. for about 5 min. A solution of (7aS,11aR)-11a-ethyl-9-oxo-7a,8,9,10,11,11a-hexahydro-7H-dibenzo[a,c]cycloheptene-3-carboxylic acid (2-methyl-pyridin-3-yl)-amide; compound with (7aR,11aS)-11a-ethyl-9-oxo-7a,8,9,10,11,11a-hexahydro-7H dibenzo[a,c]cycloheptene-3-carboxylic acid (2-methyl-pyridin-3-yl)-amide (162, R⁴=Methyl) (0.15 g, 0.40 mmol) in THF (2.0 mL) was added in one portion and the reaction stirred at rt for about 5 h. The reaction mixture was partitioned between water (25 mL) and EtOAc (25 mL). After separating the layers, the organic phase was washed with saturated aqueous NaCl (20 mL), dried over Na₂SO₄, filtered, and concentrated under reduced pressure. The residue was purified on silica gel (12 g) using a gradient of 0-5% MeOH in DCM. Collection and concentration of the appropriate fractions to yield (+/−) Compound 163 (R⁴=Methyl) (0.129 g, 83%). LC/MS, method 3, Rₜ=2.11 min, MS m/z 389 (M+H)⁺. ¹H NMR (400 MHz, CDCl₃) δ 8.66-8.45 (m, 1H), 8.37-8.32 (m, 1H), 7.83-7.66 (m, 3H), 7.54-7.48 (m, 1H), 7.41-7.28 (m, 1H), 6.45 (dd, J=12.2, 3.1 Hz, 1H), 5.94-5.84 (m, 1H), 2.98-2.87 (m, 1H), 2.76-2.65 (m, 3H), 2.61-2.53 (m, 3H), 2.44-2.36 (m, 1H), 2.30-2.19 (m, 1H), 2.15-2.03 (m, 1H), 1.94-1.81 (m, 2H), 1.68-1.41 (m, 2H), 1.27-1.18 (m, 1H), 0.94-0.86 (m, 1H), 0.85-0.77 (t, J=7.4 Hz, 3H).

Step #8: (7aS,9R,11aR)-11a-Ethyl-9-hydroxy-9-propyl-7a,8,9,10,11,11a-hexahydro-7H-dibenzo[a,c]cycloheptene-3-carboxylic acid (2-methyl-pyridin-3-yl)-amide compound with (7aR,9S,11aS)-11a-ethyl-9-hydroxy-9-propyl-7a,8,9,10,11,11a-hexahydro-7H dibenzo[a,c]cycloheptene-3-carboxylic acid (2-methyl-pyridin-3-yl)-amide (164, R⁴=Methyl, R⁵=Ethyl)

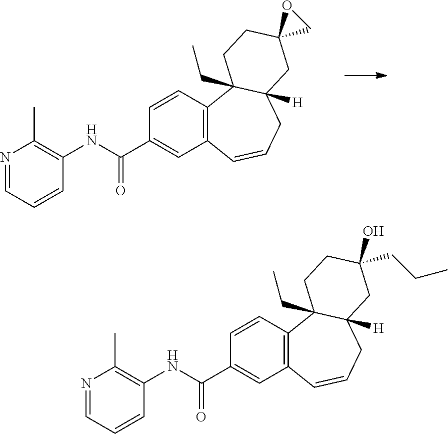

A suspension of (+/−) Compound 163 (R⁴=Methyl) (0.129 g, 0.332 mmol) and CuI (6.3 mg, 0.033 mmol) in THF (3.3 mL) was cooled to about 0° C. and then treated with ethylmagnesium bromide (0.66 mL, 2.0 mmol; 3 M solution in Et₂O) dropwise via syringe. After stirring for 5 min, the reaction mixture was quenched at 0° C. by addition of saturated aqueous NH₄Cl (10 mL), and then partitioned between EtOAc (15 mL) and water (5 mL). After separating the layers, the organic phase was washed with saturated aqueous NaCl (20 mL), dried over Na₂SO₄, filtered, and concentrated under reduced pressure. The residue was purified on silica gel (12 g) using a gradient of 0-5% MeOH in DCM. Collection and concentration of the appropriate fractions gave (7aS,9R,11aR)-11a-ethyl-9-hydroxy-9-propyl-7a,8,9,10,11,11a-hexahydro-7H-dibenzo[a,c]cycloheptene-3-carboxylic acid (2-methyl-pyridin-3-yl)-amide; compound with (7aR,9S,11aS)-11a-ethyl-9-hydroxy-9-propyl-7a,8,9,10,11,11a-hexahydro-7H-dibenzo[a,c]cycloheptene-3-carboxylic acid (2-methyl-pyridin-3-yl)-amide (164, R⁴=Methyl, R⁵=Ethyl) (0.089 g, 64%). LC/MS, method 2, Rₜ=2.18 min, MS m/z 419 (M+H)⁺. ¹H NMR (400 MHz, DMSO-d₆) δ 10.00 (s, 1H), 8.34 (dd, J=4.7, 1.6 Hz, 1H), 7.83 (d, J=2.1 Hz, 1H), 7.81-7.71 (m, 2H), 7.49 (d, J=8.4 Hz, 1H), 7.28 (dd, J=7.9, 4.7 Hz, 1H), 6.42 (dd, J=12.3, 2.8 Hz, 1H), 5.92-5.79 (m, 1H), 3.94 (s, 1H), 2.87-2.76 (m, 1H), 2.45 (s, 3H), 2.43-2.36 (m, 1H), 2.36-2.27 (m, 1H), 2.15-2.03 (m, 1H), 1.83-1.70 (m, 1H), 1.52-1.37 (m, 3H), 1.32-0.98 (m, 7H), 0.79-0.66 (m, 6H).

The chiral purification of (7aS,9R,11aR)-11a-ethyl-9-hydroxy-9-propyl-7a,8,9,10,11,11a-hexahydro-7H-dibenzo[a,c]cycloheptene-3-carboxylic acid (2-methyl-pyridin-3-yl)-amide; compound with (7aR,9S,11aS)-11a-ethyl-9-hydroxy-9-propyl-7a,8,9,10,11,11a-hexahydro-7H-dibenzo[a,c]cycloheptene-3-carboxylic acid (2-methyl-pyridin-3-yl)-amide (164, R⁴=Methyl, R⁵=Ethyl) using chiral separation method 16 yielded first Example 129, (7aS,9R,11aR)-11a-ethyl-9-hydroxy-9-propyl-7a,8,9,10,11,11a-hexahydro-7H-dibenzo[a,c]cycloheptene-3-carboxylic acid (2-methyl-pyridin-3-yl)-amide (164, R⁴=Methyl, R⁵=Ethyl) and second, Example 130, (7aR,9S,11aS)-11a-ethyl-9-hydroxy-9-propyl-7a,8,9,10,11,11a-hexahydro-7H-dibenzo[a,c]cycloheptene-3-carboxylic acid (2-methyl-pyridin-3-yl)-amide (164, R⁴=Methyl, R⁵=Ethyl). NMR and LCMS data for single isomers was essentially identical to the racemic mixture.

Additional examples, prepared in a manner similar to the preparation of Example 129 and Example 130 are listed in Table 7.

TABLE 7

| | | | | | | Chiral |
|---|---|---|---|---|---|---|
| | | | | | LC/MS | method/ |
| | Starting | Grignard | | LC/MS | R_{T}/ | Order of |
| Ex. # | Ketone | Rgt. | Product | method | MH⁺ | elution |
| 131 | Compound | Methylmagnesium- | Compound 167 | 2 | 2.31 min | Method 15/ |
| | 73 (R⁴ = | bromide | (7aS,9R,11aS) (R⁴ = | | 467 | First |
| | Phenyl) | | Phenyl) (R⁵ = | | | |
| | | | Methyl) | | | |
| 132 | Compound | Methylmagnesium- | Compound 167 | 2 | 2.31 min | Method 15/ |
| | 73 (R⁴ = | bromide | (7aR,9S,11aR) | | 467 | Second |
| | Phenyl) | | (R⁴ = Phenyl) (R⁵ = | | | |
| | | | Methyl) | | | |

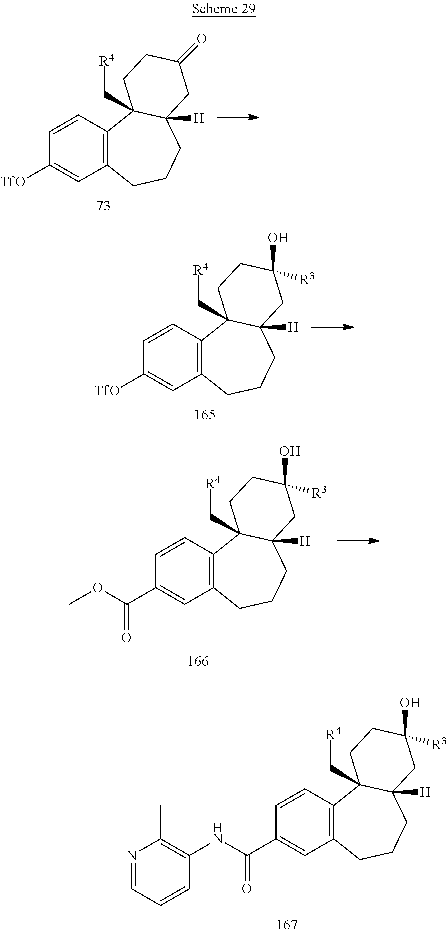

### Example 133

### (7aS,9S,11aR)-11a-Ethyl-9-hydroxy-9-(3,3,3-trifluoro-propyl)-6,7,7a,8,9,10,11,11a-octahydro-5H-dibenzo[a,c]cycloheptene-3-carboxylic acid (2-methyl-pyridin-3-yl)-amide; compound with (7aR,9R,11aS)-11a-ethyl-9-hydroxy-9-(3,3,3-trifluoro-propyl)-6,7,7a,8,9,10,11,11a-octahydro-5H-dibenzo[a,c]cycloheptene-3-carboxylic acid (2-methyl-pyridin-3-yl)-amide (167, R³=3,3,3-Trifluoro-propyl, R⁴=Methyl)

### Step #1: Trifluoro-methanesulfonic acid (7aS,9S,11aR)-11a-ethyl-9-hydroxy-9-(3,3,3-trifluoro-propyl)-6,7,7a,8,9, 10,11,11a-octahydro-5H-dibenzo[a,c]cyclohepten-3-yl ester; compound with trifluoro-methanesulfonic acid (7aR,9R,11aS)-11a-ethyl-9-hydroxy-9-(3,3,3-trifluoro-propyl)-6,7,7a,8,9,10,11,11a-octahydro-5H-dibenzo[a,c]cyclohepten-3-yl ester (165, R³=3,3,3-Trifluoro-propyl, R⁴=Methyl)

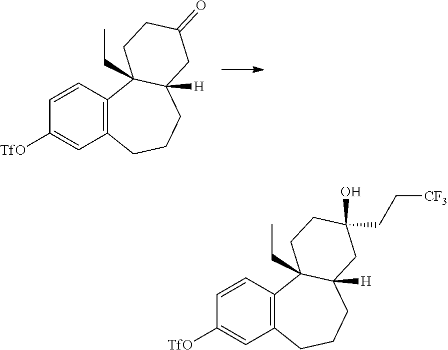

To a suspension of magnesium (0.224 g, 9.22 mmol) in Et₂O (8 mL) was added 1-iodo-3,3,3-trifluoropropane (0.90 mL, 7.7 mmol). A crystal of iodine was added, resulting in a mild exothermic reaction. After the exotherm had subsided and the mixture had cooled to rt, the reaction mixture was heated at reflux for about 30 min and then allowed to cool to rt. The solution was transferred, leaving the residual magnesium behind. A solution of trifluoro-methanesulfonic acid (7aR,11aS)-11a-ethyl-9-oxo-6,7,7a,8,9,10,11,11a-octahydro-5H-dibenzo[a,c]cyclohepten-3-yl ester; compound with trifluoro-methanesulfonic acid (7aS,11aR)-11a-ethyl-9-oxo-6,7,7a,8,9,10,11,11a-octahydro-5H-dibenzo[a,c]cyclohepten-3-yl ester (73, R⁴=Methyl) (0.600 g, 1.54 mmol) in THF (8 mL) was added dropwise, and the reaction was allowed to stir for about 30 min at rt. The reaction was quenched by addition of aqueous saturated NH₄Cl (10 mL) and after separating the layers, the aqueous phase was extracted with EtOAc (3×10 mL). The combined organic phases were washed with saturated aqueous NaCl (25 mL), dried over Na₂SO₄, filtered, and concentrated under reduced pressure. The residue was purified on silica gel (25 g) using a gradient of 0-50% EtOAc in heptane. Collection and concentration of the appropriate fractions gave the minor product, which eluted first, trifluoro-methanesulfonic acid (7aS,9S,11aR)-11a-ethyl-9-hydroxy-9-(3,3,3-trifluoro-propyl)-6,7,7a,8,9,10,11,11a-octahydro-5H-dibenzo[a,c]cyclohepten-3-yl ester; compound with trifluoro-methanesulfonic acid (7aR,9R,11aS)-11a-ethyl-9-hydroxy-9-(3,3,3-trifluoro-propyl)-6,7,7a,8,9,10,11,11a-octahydro-5H-dibenzo[a,c]cyclohepten-3-yl ester (165, R³=3,3,3-Trifluoro-propyl, R⁴=Methyl) (0.144 g, 19%). LC/MS, method 3, Rₜ=3.26 min. MS m/z 547 (M+OAc)⁻. ¹H NMR (400 MHz, DMSO-d₆) δ 7.40 (d, J=8.8 Hz, 1H), 7.30-7.20 (m, 2H), 4.37 (s, 1H), 2.99-2.79 (m, 2H), 2.43-1.95 (m, 5H), 1.85-1.48 (m, 7H), 1.50-1.24 (m, 5H), 0.54 (t, J=7.3 Hz, 3H).

### Step #2: (7aS,9S,11aR)-11a-Ethyl-9-hydroxy-9-(3,3,3-trifluoro-propyl)-6,7,7a,8,9,10,11,11a-octahydro-5H-dibenzo[a,c]cycloheptene-3-carboxylic acid methyl ester; compound with (7aR,9R,11aS)-11a-ethyl-9-hydroxy-9-(3,3,3-trifluoro-propyl)-6,7,7a,8,9,10,11,11a-octahydro-5H-dibenzo[a,c]cycloheptene-3-carboxylic acid methyl ester (166, R³=3,3,3-Trifluoro-propyl, R⁴=Methyl)

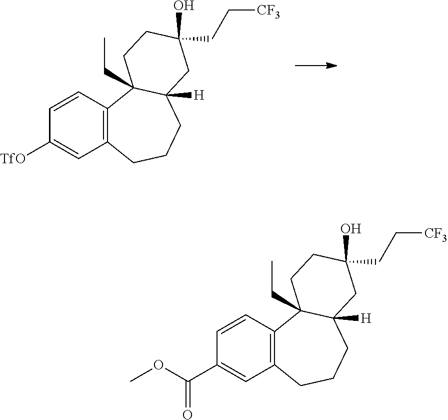

A suspension of trifluoro-methanesulfonic acid (7aS,9S,11aR)-11a-ethyl-9-hydroxy-9-(3,3,3-trifluoro-propyl)-6,7,7a,8,9,10,11,11a-octahydro-5H-dibenzo[a,c]cyclohepten-3-yl ester; compound with trifluoro-methanesulfonic acid (7aR,9R,11aS)-11a-ethyl-9-hydroxy-9-(3,3,3-trifluoro-propyl)-6,7,7a,8,9,10,11,11a-octahydro-5H-dibenzo[a,c]cyclohepten-3-yl ester (165, R³=3,3,3-Trifluoro-propyl, R⁴=Methyl) (0.144 g, 0.295 mmol), Pd₂(dba)₃ (8.1 mg, 8.8 μmol), and Xantphos (0.017 g, 0.029 mmol) in DMF (3.0 mL) was degassed under vacuum for about 20 minutes. Carbon monoxide from a balloon was added and this cycle was repeated two more times before the reaction was left to stir under an atmosphere of carbon monoxide. TEA (0.16 mL, 1.2 mmol) and MeOH (0.14 mL, 3.5 mmol) were added sequentially via syringe, and the reaction mixture was heated at about 80° C. for about 15 h. The reaction mixture was concentrated under reduced pressure and then diluted with and concentrated from toluene multiple times (3×10 mL). The residue was adsorbed onto silica gel (1.5 g) and then purified on silica gel (12 g) using a gradient of 0-50% EtOAc in heptane. Collection and concentration of the appropriate fractions gave (7aS,9S,11aR)-11a-ethyl-9-hydroxy-9-(3,3,3-trifluoro-propyl)-6,7,7a,8,9,10,11,11a-octahydro-5H-dibenzo[a,c]cycloheptene-3-carboxylic acid methyl ester; compound with (7aR,9R,11aS)-11a-ethyl-9-hydroxy-9-(3,3,3-trifluoro-propyl)-6,7,7a,8,9,10,11,11a-octahydro-5H-dibenzo[a,c]cycloheptene-3-carboxylic acid methyl ester (166, R³=3,3,3-Trifluoro-propyl, R⁴=Methyl) (0.036 g, 31%). LC/MS, method 3, Rₜ=3.03 min. MS m/z 457 (M+OAc)⁻. ¹H NMR (400 MHz, DMSO-d₆) δ 7.74 (dd, J=8.2, 2.1 Hz, 1H), 7.69 (d, J=2.1 Hz, 1H), 7.40 (d, J=8.4 Hz, 1H), 4.31 (s, 1H), 3.83 (s, 3H), 3.00-2.81 (m, 2H), 2.31-2.01 (m, 4H), 1.85-1.50 (m, 7H), 1.52-1.17 (m, 6H), 0.56 (t, J=7.3 Hz, 3H).

### Step #3: (7aS,9S,11aR)-11a-Ethyl-9-hydroxy-9-(3,3,3-trifluoro-propyl)-6,7,7a,8,9,10,11,11a-octahydro-5H-dibenzo[a,c]cycloheptene-3-carboxylic acid (2-methyl-pyridin-3-yl)-amide; compound with (7aR,9R,11aS)-11a-ethyl-9-hydroxy-9-(3,3,3-trifluoro-propyl)-6,7,7a,8,9,10,11,11a-octahydro-5H-dibenzo[a,c]cycloheptene-3-carboxylic acid (2-methyl-pyridin-3-yl)-amide (167, R³=3,3,3-Trifluoro-propyl, R⁴=Methyl)

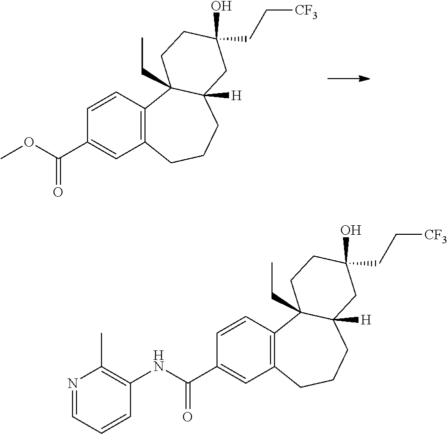

A suspension of (7aS,9S,11aR)-11a-ethyl-9-hydroxy-9-(3,3,3-trifluoro-propyl)-6,7,7a,8,9,10,11,11a-octahydro-5H-dibenzo[a,c]cycloheptene-3-carboxylic acid methyl ester; compound with (7aR,9R,11aS)-11a-ethyl-9-hydroxy-9-(3,3,3-trifluoro-propyl)-6,7,7a,8,9,10,11,11a-octahydro-5H-dibenzo[a,c]cycloheptene-3-carboxylic acid methyl ester (166, R³=3,3,3-Trifluoro-propyl, R⁴=Methyl) (0.036 g, 0.090 mmol) and 3-amino-2-picoline (0.017 g, 0.16 mmol) in toluene (1.8 mL) was treated with LiHMDS (0.27 mL, 0.27 mmol, 1 M solution in THF). The resulting suspension was allowed to stir at rt for about 5 min, and then the reaction was quenched by addition of saturated aqueous NH₄Cl (2 mL). After separating the layers, the aqueous phase was extracted with EtOAc (3×5 mL). The combined organic phases were dried over Na₂SO₄, filtered, and concentrated under reduced pressure. The residue was purified on silica gel (4 g) using a gradient of 0-5% MeOH in DCM. Collection and concentration of the appropriate fractions gave (7aS,9S,11aR)-11a-ethyl-9-hydroxy-9-(3,3,3-trifluoro-propyl)-6,7,7a,8,9,10,11,11a-octahydro-5H-dibenzo[a,c]cycloheptene-3-carboxylic acid (2-methyl-pyridin-3-yl)-amide; compound with (7aR,9R,11aS)-11a-ethyl-9-hydroxy-9-(3,3,3-trifluoro-propyl)-6,7,7a,8,9,10,11,11a-octahydro-5H-dibenzo[a,c]cycloheptene-3-carboxylic acid (2-methyl-pyridin-3-yl)-amide (167, R³=3,3,3-Trifluoro-propyl, R⁴=Methyl) (0.035 g, 82%). LC/MS, method 2, Rₜ=2.48 min, MS m/z 475 (M+H)⁺. ¹H NMR (400 MHz, DMSO-d₆) δ 9.95 (s, 1H), 8.33 (dd, J=4.8, 1.6 Hz, 1H), 7.78 (dd, J=8.1, 2.1 Hz, 1H), 7.72 (dd, J=8.0, 1.7 Hz, 2H), 7.40 (d, J=8.4 Hz, 1H), 7.27 (dd, J=8.0, 4.7 Hz, 1H), 4.34 (s, 1H), 3.04-2.83 (m, 2H), 2.47-2.40 (m, 4H), 2.35-2.02 (m, 4H), 1.87-1.56 (m, 7H), 1.55-1.13 (m, 5H), 0.60 (t, J=7.3 Hz, 3H).

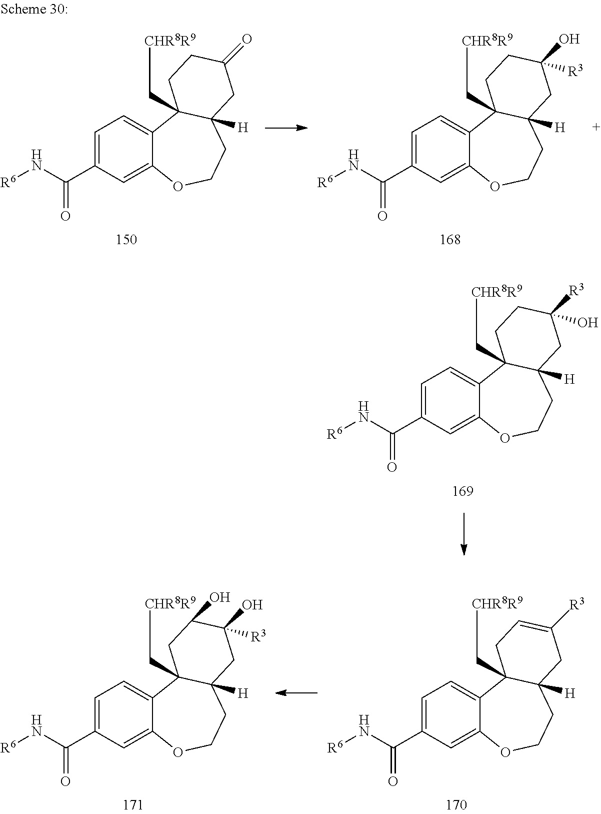

### Example #134

### (7aS,9R,11aR)-11a-Ethyl-9-hydroxy-N-(2-methylpyridin-3-yl)-9-phenyl-6,7,7a,8,9,10,11,11a-octahydrodibenzo[b,d]oxepine-3-carboxamide; compound with (7aR,9S,11aS)-11a-ethyl-9-hydroxy-N-(2-methylpyridin-3-yl)-9-phenyl-6,7,7a,8,9,10,11,11a-octahydrodibenzo[b,d]oxepine-3-carboxamide (168, R³=Phenyl, R⁶=2-Methylpyridin-3-yl, R⁸=H, R⁹=H)

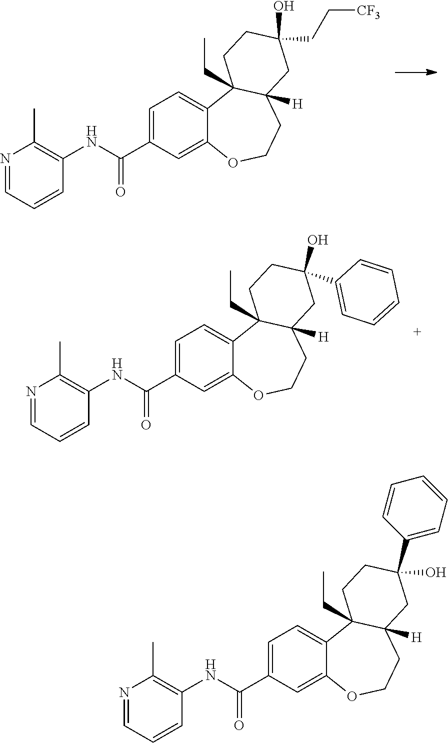

A solution of (7aS,11aR)-11a-ethyl-N-(2-methylpyridin-3-yl)-9-oxo-6,7,7a,8,9,10,11,11a-octahydrodibenzo[b,d]oxepine-3-carboxamide; compound with (7aR,11aS)-11a-ethyl-N-(2-methylpyridin-3-yl)-9-oxo-6,7,7a,8,9,10,11,11a-octahydro dibenzo[b,d]oxepine-3-carboxamide (150, R⁶=2-Methylpyridin-3-yl, R⁸=H, R⁹=H) (0.100 g, 0.264 mmol) in THF (4 mL) was cooled to about 5° C. then phenylmagnesium bromide (0.79 mL, 0.79 mmol, 1 M solution in THF) was added keeping the internal temperature of the mixture below about 10° C. After about 1 h the reaction was quenched with saturated NH₄Cl (−3 mL) then diluted with water (15 mL) and extracted with EtOAc (25 mL). The organic solution was dried over MgSO₄, filtered and concentrated under reduced pressure. The material was purified on silica gel (4 g) using a gradient of 0%-100% EtOAc in heptane. Fractions of the second peak (major component) were combined and concentrated under reduced pressure to yield (7aS,9S,11aR)-11a-ethyl-9-hydroxy-N-(2-methylpyridin-3-yl)-9-phenyl-6,7,7a,8,9,10,11,11a-octahydrodibenzo[b,d]oxepine-3-carboxamide; compound with (7aR,9R,11aS)-11a-ethyl-9-hydroxy-N-(2-methylpyridin-3-yl)-9-phenyl-6,7,7a,8,9,10,11,11 a-octahydrodibenzo[b,d]oxepine-3-carboxamide (169, R³=Phenyl, R⁶=2-Methylpyridin-3-yl, R⁸=H, R⁹=H) (0.088 g, 73%). LC/MS, method 3, Rₜ=2.24 min, MS m/z 457 (M+H)⁺. Fractions of the first peak (minor component) were combined and concentrated under reduced pressure. The material was purified further on silica gel (4 g) using a gradient of 10-100% EtOAc in heptane. Fractions with pure desired material were combined and concentrated under reduced pressure. The material was triturated with heptane (−5 mL) then filtered and dried under vacuum at about 65° C. to yield (7aS,9R,11aR)-11a-ethyl-9-hydroxy-N-(2-methylpyridin-3-yl)-9-phenyl-6,7,7a,8,9,10,11,11a-octahydrodibenzo[b,d]oxepine-3-carboxamide; compound with (7aR,9S,11aS)-11a-ethyl-9-hydroxy-N-(2-methylpyridin-3-yl)-9-phenyl-6,7,7a,8,9,10,11, 11 a-octahydrodibenzo[b,d]oxepine-3-carboxamide (168, R³=Phenyl, R⁶=2-Methylpyridin-3-yl, R⁸=H, R⁹=H) (0.0053 g, 4%). LC/MS, method 2, Rₜ=2.15 min, MS m/z 457 (M+H)⁺. ¹H NMR (400 MHz, DMSO-d₆) δ 10.01 (s, 1H), 8.33 (dd, J=4.8, 1.6 Hz, 1H), 7.74-7.68 (m, 2H), 7.57 (d, J=2.0 Hz, 1H), 7.49 (d, J=8.4 Hz, 1H), 7.34-7.19 (m, 5H), 7.19-7.10 (m, 1H), 4.93 (s, 1H), 4.28-4.17 (m, 1H), 3.77-3.71 (m, 1H), 2.71-2.53 (m, 2H), 2.44 (s, 3H), 2.34-2.26 (m, 1H), 2.21-2.07 (m, 1H), 2.07-1.82 (m, 2H), 1.81-1.68 (m, 2H), 1.66-1.41 (m, 2H), 1.41-1.30 (m, 1H), 0.62 (t, J=7.6 Hz, 3H).

### Example #135

### (7aS,9R,10R,11aR)-11a-Ethyl-9,10-dihydroxy-N-(2-methylpyridin-3-yl)-9-phenyl-6,7,7a,8,9,10,11,11a-octahydrodibenzo[b,d]oxepine-3-carboxamide; compound with (7aR,9S,10S,11aS)-11a-ethyl-9,10-dihydroxy-N-(2-methylpyridin-3-yl)-9-phenyl-6,7,7a,8,9,10,11,11a-octahydrodibenzo[b,d]oxepine-3-carboxamide (171, R³=Phenyl, R⁶=2-Methylpyridin-3-yl, R⁸=H, R⁹=H)

### Step #1: (7aR,11aR)-11a-Ethyl-N-(2-methylpyridin-3-yl)-9-phenyl-6,7,7a,8,11,11a-hexahydrodibenzo[b,d]oxepine-3-carboxamide; compound with (7aS,11aS)-11a-ethyl-N-(2-methylpyridin-3-yl)-9-phenyl-6,7,7a,8,11,11a-hexahydro dibenzo[b,d]oxepine-3-carboxamide (170, R³=Phenyl, R⁶=2-Methylpyridin-3-yl, R⁸=H, R⁹=H)

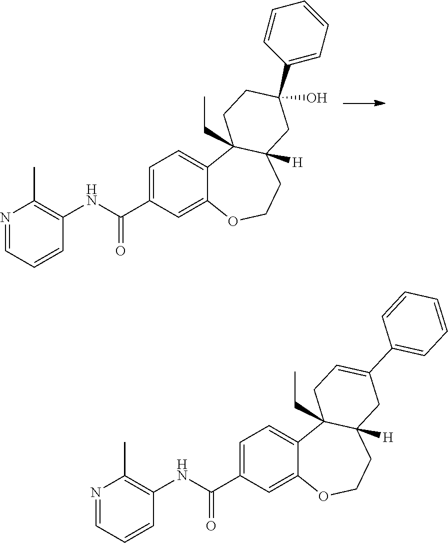

A mixture of (7aS,9S,11aR)-11a-ethyl-9-hydroxy-N-(2-methylpyridin-3-yl)-9-phenyl-6,7,7a,8,9,10,11,11a-octahydrodibenzo[b,c]oxepine-3-carboxamide; compound with (7aR,9R,11aS)-11a-ethyl-9-hydroxy-N-(2-methylpyridin-3-yl)-9-phenyl-6,7,7a,8,9,10,11,11a-octahydrodibenzo[b,d]oxepine-3-carboxamide (169, R³=phenyl, R⁶=2-Methylpyridin-3-yl, R⁸═H, R⁹=H) (0.088 g, 0.193 mmol) and KHSO₄ (0.055 g, 0.41 mmol) in toluene (6 mL) was heated at about 110° C. for about 3 h. The mixture was cooled and diluted with toluene (15 mL). 4-Methylbenzenesulfonic acid hydrate (0.077 g, 0.405 mmol) was added, the flask was fitted with a Dean-Stark apparatus and the mixture heated to reflux for about 90 min The mixture was cooled and concentrated under reduced pressure. The material was treated with water (15 mL) then saturated aqueous NaHCO₃ (˜4 mL). The mixture was extracted with EtOAc (2×15 mL). The combined organics were dried over MgSO₄, filtered and concentrated under reduced pressure. The material was triturated with heptane (˜5 mL) then the solvents were removed under reduced pressure to yield (7aR,11aR)-11a-ethyl-N-(2-methylpyridin-3-yl)-9-phenyl-6,7,7a,8,11,11a-hexahydrodibenzo[b,d]oxepine-3-carboxamide; compound with (7aS,11aS)-11a-ethyl-N-(2-methylpyridin-3-yl)-9-phenyl-6,7,7a,8,11,11a-hexahydrodibenzo[b,d]oxepine-3-carboxamide (170, R³=Phenyl, R⁶=2-Methylpyridin-3-yl, R⁸=H, R⁹=H) (0.050 g, 60%). LC/MS, method 3, Rₜ=2.75 min, MS m/z 439 (M+H)⁺. ¹H NMR (400 MHz, CDCl₃) δ 8.46 (s, 1H), 8.32 (dd, J=4.9, 1.5 Hz, 1H), 7.67-7.62 (m, 1H), 7.50 (d, J=2.0 Hz, 1H), 7.45 (dd, J=8.1, 2.1 Hz, 1H), 7.33-7.23 (m, 5H), 7.23-7.17 (m, 2H), 6.30-6.26 (m, 1H), 4.42-4.33 (m, 1H), 3.93-3.86 (m, 1H), 2.96-2.77 (m, 2H), 2.74-2.55 (m, 5H), 2.37-2.24 (m, 2H), 2.22-2.18 (m, 1H), 1.67-1.55 (m, 2H), 0.67 (t, J=7.2 Hz, 3H).

### Step #2: (7aS,9R,10R,11aR)-11a-Ethyl-9,10-dihydroxy-N-(2-methylpyridin-3-yl)-9-phenyl-6,7,7a,8,9,10,11,11a-octahydrodibenzo[b,d]oxepine-3-carboxamide; compound with (7aR,9S,10S,11aS)-11a-ethyl-9,10-dihydroxy-N-(2-methylpyridin-3-yl)-9-phenyl-6,7,7a,8,9,10,11,11a-octahydrodibenzo[b,d]oxepine-3-carboxamide (171, R³=Phenyl, R⁶=2-Methylpyridin-3-yl, R⁸=H, R⁹=H)

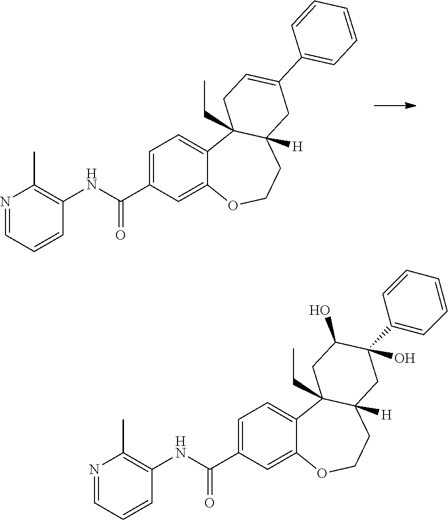

(7aR,11aR)-11a-Ethyl-N-(2-methylpyridin-3-yl)-9-phenyl-6,7,7a,8,11,11a-hexahydrodibenzo[b,d]oxepine-3-carboxamide; compound with (7aS,11aS)-11a-ethyl-N-(2-methylpyridin-3-yl)-9-phenyl-6,7,7a,8,11,11a-hexahydrodibenzo[b,d]oxepine-3-carboxamide (170, R³=Phenyl, R⁶=2-Methylpyridin-3-yl, R⁸=H, R⁹=H) (0.050 g, 0.114 mmol) in THF (6 mL) and water (1 mL) was treated with NMO (0.027 g, 0.228 mmol) and osmium (VIII) oxide (0.174 g, 0.017 mmol, 2.5 wt % in tBuOH). After about 2 h, osmium (VIII) oxide (0.175 g, 0.028 mmol, 4 wt % in water) was added and the mixture was stirred at rt for about 18 h. The reaction mixture was diluted with water (15 mL) then EtOAc (20 mL) and saturated aqueous NaHCO₃ (4 mL) were added to the mixture. The layers were separated then the aqueous layer was extracted with EtOAc (20 mL). The combined organic solutions were dried over MgSO₄, filtered and concentrated under reduced pressure. The material was purified on silica gel (4 g) using a gradient of 50-100% EtOAc in heptane. Fractions containing product were combined and concentrated under reduced pressure, then treated with EtOAc (2 mL). The solid formed was collected by filtration and dried under vacuum at about 60° C. to yield (7aS,9R,10R,11aR)-11a-ethyl-9,10-dihydroxy-N-(2-methylpyridin-3-yl)-9-phenyl-6,7,7a,8,9,10,11,11a-octahydrodibenzo[b,d]oxepine-3-carboxamide; compound with (7aR,9S,10S,11aS)-11a-ethyl-9,10-dihydroxy-N-(2-methylpyridin-3-yl)-9-phenyl-6,7,7a,8,9,10,11,11a-octahydrodibenzo[b,d]oxepine-3-carboxamide (171, R³=Phenyl, R⁶=2-Methylpyridin-3-yl, R⁸=H, R⁹=H) (33.4 mg, 62%). LC/MS, method 2, Rₜ=1.93 min, MS m/z 473 (M+H)⁺. ¹H NMR (400 MHz, DMSO-d₆) δ 10.03 (s, 1H), 8.34 (dd, J=4.8, 1.6 Hz, 1H), 7.78-7.71 (m, 2H), 7.57 (m, 2H), 7.32-7.20 (m, 5H), 7.17-7.10 (m, 1H), 4.66 (s, 1H), 4.50 (d, J=6.3 Hz, 1H), 4.19 (d, J=12.4 Hz, 1H), 4.16-4.08 (m, 1H), 3.71-3.65 (m, 1H), 2.59-2.49 (m, 2H), 2.45 (s, 3H), 2.42-2.31 (m, 1H), 2.14-2.05 (m, 1H), 1.89-1.78 (m, 1H), 1.69-1.63 (m, 1H), 1.60-1.47 (m, 2H), 1.36-1.32 (m, 1H), 0.62 (t, J=7.4 Hz, 3H).

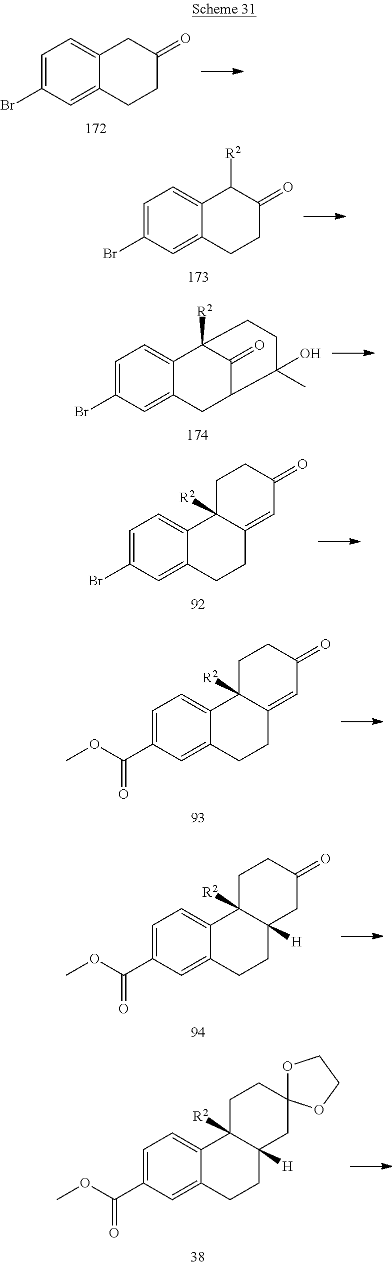

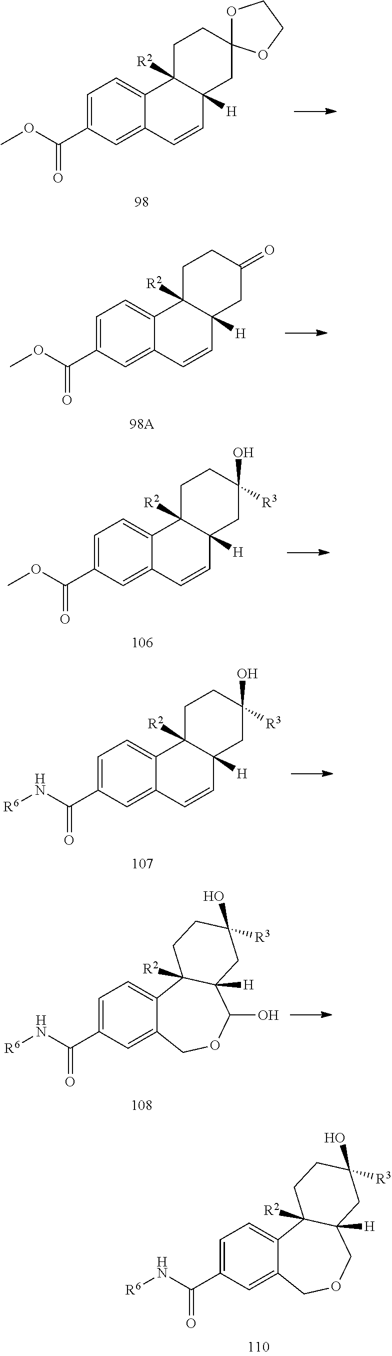

### Example #136

### (7aS,9R,11aR)-11a-Ethyl-9-propyl-9-hydroxy-N-(2-methylpyridin-3-yl)-5,7,7a,8,9,10,11,11a-octahydrodibenzo[c,e]oxepine-3-carboxamide; compound with (7aR,9S,11aS)-11a-ethyl-9-propyl-9-hydroxy-N-(2-methylpyridin-3-yl)-5,7,7a,8,9,10,11,11a-octahydrodibenzo[c,e]oxepine-3-carboxamide (110, R²=Ethyl, R³=Propyl, R⁶=2-Methylpyridin-2-yl)

### Step #1: 6-Bromo-1-ethyl-3,4-dihydronaphthalen-2(1H)-one (173, R²=Ethyl)

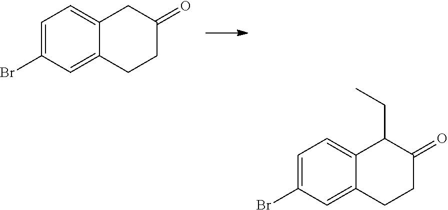

A solution of 6-bromo-3,4-dihydronaphthalen-2(1H)-one (172) (49.0 g, 218 mmol) [ECA], pyrrolidine (40.0 mL, 479 mmol) and toluene (400 mL) under a nitrogen atmosphere was heated using a Dean-Stark apparatus at reflux for about 20 h. The solvents were removed under reduced pressure then dried under reduced pressure for about 16 h to afford a brown solid. The residue was placed under a nitrogen atmosphere. Iodoethane (260 mL, 3.25 mol) was added in one portion. The reaction vessel was evacuated and then back-filled with nitrogen three times. The mixture was warmed to about 70° C. After about 20 h, the mixture was allowed to cool to rt. The volatiles were removed under reduced pressure. The residue was concentrated under reduced pressure from EtOAc (300 mL) and then heptane (2×300 mL). The material was dried under reduced pressure for about 16 h to afford a brown solid. A biphasic mixture of a quarter of the residue (24 g), degassed toluene (200 mL) and water (200 mL) was evacuated under reduced pressure and back-filled with nitrogen five times then warmed to about 100° C. After about 5 h, the mixture was allowed to cool to rt. After about 15 h, the mixture was poured into 1 M aqueous HCl (220 mL) and EtOAc (400 mL). The layers were vigorously mixed then separated. The organics were washed with water (50 mL) and saturated aqueous NaCl (100 mL). The aqueous phases were extracted with EtOAc (2×100 mL). The combined organics were dried over MgSO₄, filtered, and concentrated under reduced pressure. The residue was purified on silica gel (330 g) using a gradient of 0-8% EtOAc in heptane. The hydrolysis was repeated as above for the remainder of the material. The fractions containing product from the four runs were combined and concentrated under reduced pressure to afford 6-bromo-1-ethyl-3,4-dihydronaphthalen-2(1H)-one (173, R²=Ethyl) (41.9 g, 76%) as a light yellow oil. LC/MS, method 3, Rₜ=2.47 min, MS m/z 251 and 253 (M−H)⁻. ¹H NMR (400 MHz, DMSO-d₆) δ 7.49 (d, J=2.1 Hz, 1H), 7.42 (dd, J=8.2, 2.2 Hz, 1H), 7.15 (d, J=8.3 Hz, 1H), 3.37 (t, J=6.6 Hz, 1H), 3.13-2.95 (m, 2H), 2.51-2.45 (m, 2H), 1.87-1.75 (m, 2H), 0.80 (t, J=7.4 Hz, 3H).

### Step #2: (+/−) Compound 174 (R²=Ethyl)

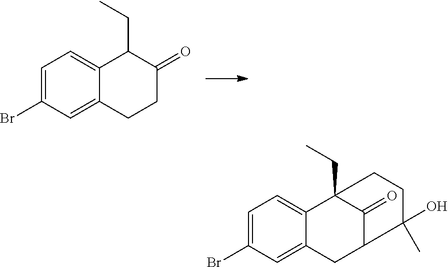

4 Å molecular sieves (50 g) were added to a solution of 6-bromo-1-ethyl-3,4-dihydronaphthalen-2(1H)-one (173, R²=Ethyl) (22.0 g, 87.0 mmol), (S)-1-phenylethylamine (12.2 mL, 95.8 mmol), and toluene (140 mL) under a nitrogen atmosphere. The reaction vessel was evacuated then back-filled with nitrogen three times. The reaction vessel was sealed and the mixture was warmed to about 60° C. After about 22 h, a nitrogen line was attached and the mixture was cooled to about 0° C. But-3-en-2-one (8.40 mL, 104 mmol) was added dropwise. After about 5 min, the ice bath was removed. After about 30 min, the reaction vessel was sealed and the mixture was warmed to about 50° C. After about 19 h, the mixture was allowed to cool to rt. The mixture was filtered rinsing with toluene (800 mL). 2 M aqueous H₂SO₄ (500 mL) was added. The biphasic solution was stirred at about 50° C. for about 22 h. The mixture was allowed to cool to rt. EtOAc (500 mL) was added and the layers were separated. The organics were washed with water (300 mL), a solution of 50% saturated aqueous NaHCO₃ in water (300 mL), and saturated aqueous NaCl (300 mL). The aqueous layers were extracted with EtOAc (200 mL). The combined organics were dried over Na₂SO₄, filtered, and concentrated under reduced pressure. The residue was purified on silica gel (220 g) using a gradient of 0-40% EtOAc in heptane. The mixed fractions were collected and concentrated under reduced pressure. The material was purified as above. The fractions containing product were combined and concentrated under reduced pressure to afford (+/−) Compound 174 (R²=Ethyl) (15.4 g, 55%) as a very light tan foam. LC/MS, method 3, Rₜ=2.35 min, MS m/z 323 and 325 (M+H)⁺. ¹H NMR (400 MHz, DMSO-d₆) δ 7.42 (dd, J=8.4, 2.2 Hz, 1H), 7.38 (d, J=2.1 Hz, 1H), 7.13 (d, J=8.5 Hz, 1H), 4.63 (s, 1H), 3.23-3.08 (m, 2H), 2.44-2.38 (m, 1H), 2.05-1.89 (m, 2H), 1.81-1.69 (m, 1H), 1.44-1.31 (m, 2H), 1.31-1.11 (m, 1H), 1.21 (s, 3H), 0.72 (t, J=7.2 Hz, 3H). Chiral analysis, analytical chiral chromatography method A, UV trace (230-420 nm): Peak 1: Rₜ=3.08 min, 12% of integrated area. Peak 2: Rₜ=3.26 min, 88% of integrated area.

### Step #3: (R)-7-Bromo-4a-ethyl-4,4a,9,10-tetrahydrophenanthren-2(3H)-one; compound with (S)-7-bromo-4a-ethyl-4,4a,9,10-tetrahydrophenanthren-2(3H)-one (92, R²=Ethyl)

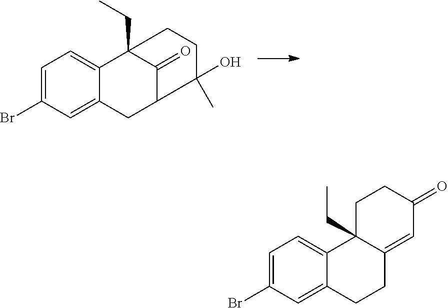

4-Methylbenzenesulfonic acid hydrate (0.906 g, 4.76 mmol) was added to a solution of (+/−) Compound 174 (R²=Ethyl) (15.4 g, 47.6 mmol) and toluene (600 mL). The reaction vessel was evacuated and then back-filled with nitrogen ten times. The reaction solution was warmed to reflux for about 4 h. After allowing to cool to rt, saturated aqueous NaHCO₃ (300 mL) and EtOAc (400 mL) were added. The layers were separated and the organics were washed with saturated aqueous NaCl (200 mL), dried over MgSO₄, filtered and concentrated under reduced pressure. The residue was purified on silica gel (330 g) using a gradient of 5-16% EtOAc in heptane. The fractions containing product were combined and concentrated under reduced pressure to afford (R)-7-bromo-4a-ethyl-4,4a,9,10-tetrahydrophenanthren-2(3H)-one; compound with (S)-7-bromo-4a-ethyl-4,4a,9,10-tetrahydrophenanthren-2(3H)-one (92, R²=Ethyl) (12.6 g, 87%) as a yellow oil. LC/MS, method 3, Rₜ=2.56 min, MS m/z 305 and 307 (M+H)⁺. ¹H NMR (400 MHz, CDCl₃) δ 7.36 (dd, J=8.5, 2.2 Hz, 1H), 7.29-7.27 (m, 1H), 7.15 (d, J=8.5 Hz, 1H), 5.96-5.94 (m, 1H), 3.05-2.95 (m, 1H), 2.89-2.79 (m, 1H), 2.79-2.64 (m, 2H), 2.63-2.55 (m, 1H), 2.52-2.43 (m, 1H), 2.43-2.35 (m, 1H), 2.09-1.89 (m, 3H), 0.82 (t, J=7.5 Hz, 3H).

### Step #4: (R)-Methyl 4b-ethyl-7-oxo-4b,5,6,7,9,10-hexahydrophenanthrene-2-carboxylate; compound with (S)-methyl 4b-ethyl-7-oxo-4b,5,6,7,9,10-hexahydrophenanthrene-2-carboxylate (93, R²=Ethyl)

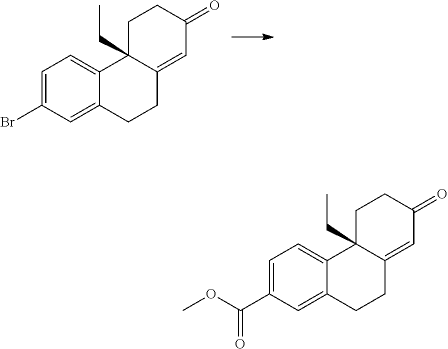

1,1′-Bis(diphenylphosphino)ferrocenedichloropalladium(II) dichloromethane adduct [Frontier] (0.704 g, 0.862 mmol), (R)-7-bromo-4a-ethyl-4,4a,9,10-tetrahydrophenanthren-2(3H)-one; compound with (S)-7-bromo-4a-ethyl-4,4a,9,10-tetrahydrophenanthren-2(3H)-one (92, R²=Ethyl) (26.3 g, 86.0 mmol), triethylamine (24.0 mL, 172 mmol) and MeOH (260 mL) were added to a Parr reactor under a nitrogen atmosphere. The reactor was purged with nitrogen and then carbon monoxide. The reaction mixture was placed under about 60 psi of carbon monoxide and then agitated for about 5 h at about 100° C. After cooling to rt, the reaction mixture was filtered through a polypropylene filter funnel with diatomaceous earth/polyethylene frit disc rinsing with MeOH. The volatiles were removed under reduced pressure. The residue was purified on silica gel (330 g) using DCM as eluant. The fractions containing product were combined and concentrated under reduced pressure to afford (R)-methyl 4b-ethyl-7-oxo-4b,5,6,7,9,10-hexahydrophenanthrene-2-carboxylate; compound with (S)-methyl 4b-ethyl-7-oxo-4b,5,6,7,9,10-hexahydrophenanthrene-2-carboxylate (93, R²=Ethyl) (22.4 g, 91%) as a brown oil. LC/MS, method 3, Rₜ=2.22 min, MS m/z 285 (M+H)⁺. ¹H NMR (400 MHz, CDCl₃) δ 7.91-7.86 (m, 1H), 7.82-7.79 (m, 1H), 7.35 (d, J=8.3 Hz, 1H), 5.96 (s, 1H), 3.91 (s, 3H), 3.14-3.05 (m, 1H), 2.95-2.84 (m, 1H), 2.81-2.67 (m, 2H), 2.67-2.58 (m, 1H), 2.54-2.39 (m, 2H), 2.12-1.95 (m, 3H), 0.82 (t, J=7.5 Hz, 3H).

### Step #5: (4bR,8aS)-Methyl 4b-ethyl-7-oxo-4b,5,6,7,8,8a,9,10-octahydrophenanthrene-2-carboxylate; compound with (4bS,8aR)-methyl 4b-ethyl-7-oxo-4b,5,6,7,8,8a,9,10-octahydrophenanthrene-2-carboxylate (94, R²=Ethyl)

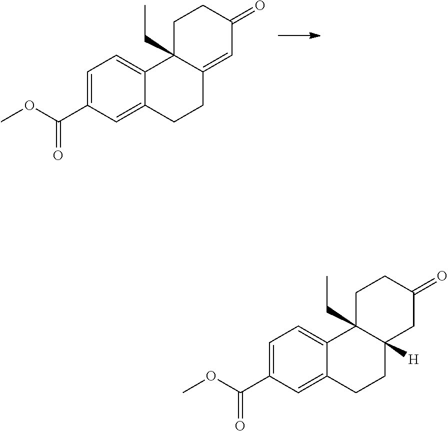

(R)-Methyl 4b-ethyl-7-oxo-4b,5,6,7,9,10-hexahydrophenanthrene-2-carboxylate; compound with (S)-methyl 4b-ethyl-7-oxo-4b,5,6,7,9,10-hexahydrophenanthrene-2-carboxylate (93, R²=Ethyl) (20.2 g, 71.0 mmol), 5% Pd/C (5.5 g) [Johnson Matthey], THF (160 mL) and pyridine (40 mL) were added under nitrogen to a 1.8 L SS pressure bottle. The reactor was purged with nitrogen and then hydrogen. The reaction mixture was placed under about 30 psi of hydrogen and then agitated for about 30 h at rt. The reaction mixture was filtered through a Buchner funnel containing a GF/F glass fiber filter rinsing with THF. The volatiles were removed under reduced pressure. The residue was dissolved in DCM (400 mL) and then washed with 0.2 M aqueous CuSO₄ (3×200 mL). The organics were dried over Na₂SO₄, filtered, and concentrated under reduced pressure. The residue was dissolved in MeOH and then concentrated under reduced pressure. The residue was dissolved in a minimum amount of MeOH then cooled to about 0° C. for about 20 h. The solids were collected by filtration rinsing with cold MeOH. The solids were dried under reduced pressure at about 50° C. for about 30 min to afford (4bR,8aS)-methyl 4b-ethyl-7-oxo-4b,5,6,7,8,8a,9,10-octahydrophenanthrene-2-carboxylate; compound with (4bS,8aR)-methyl 4b-ethyl-7-oxo-4b,5,6,7,8,8a,9,10-octahydrophenanthrene-2-carboxylate (94, R²=Ethyl) (5.57 g, 25%) as an off-white solid. Chiral analysis, analytical chiral chromatography method B, UV trace (230-420 nm): Peak 1: Rₜ=4.01 min, 50% of integrated area. Peak 2: Rₜ=4.22 min, 50% of integrated area. The mother liquor was concentrated under reduced pressure. The residue was purified on silica gel (330 g) using a gradient of 0-5% EtOAc in DCM. The fractions containing product were combined and concentrated under reduced pressure to afford (4bR,8aS)-methyl 4b-ethyl-7-oxo-4b,5,6,7,8,8a,9,10-octahydrophenanthrene-2-carboxylate (94, R²=Ethyl) (12.9 g, 58%) as an oil. LC/MS, method 3, Rₜ=2.38 min, MS m/z 287 (M+H)⁺. ¹H NMR (400 MHz, DMSO-d₆) δ 7.74 (dd, J=8.2, 1.8 Hz, 1H), 7.70 (d, J=1.6 Hz, 1H), 7.51 (d, J=8.2 Hz, 1H), 3.83 (s, 3H), 2.88-2.81 (m, 2H), 2.49-2.44 (m, 1H), 2.42-2.23 (m, 3H), 2.13-1.69 (m, 6H), 1.62-1.50 (m, 1H), 0.70 (t, J=7.4 Hz, 3H). Chiral analysis, analytical chiral chromatography method B, UV trace (230-420 nm): Peak 1: Rₜ=4.01 min, 3% of integrated area. Peak 2: Rₜ=4.22 min, 97% of integrated area.

### Step #6: (4a′R,10a′S)-Methyl 4a′-ethyl-3′,4′,4a′,9′,10′,10a′-hexahydro-1′H-spiro[[1,3]dioxolane-2,2′-phenanthrene]-7′-carboxylate; compound with (4a′S,10a′R)-Methyl 4a′-ethyl-3′,4′,4a′,9′,10′,10a′-hexahydro-1′H-spiro[[1,3]dioxolane-2,2′-phenanthrene]-7′-carboxylate (38, R²=Ethyl)

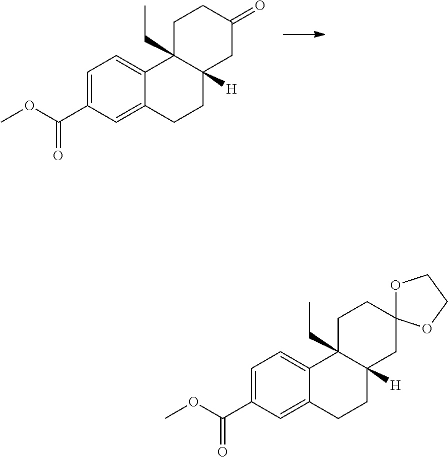

Ethylene glycol (8.02 g, 129 mmol) and toluene-4-sulfonic acid hydrate (0.492 g, 2.58 mmol) were respectively added, each in one portion, to a solution of (4bR,8aS)-methyl 4b-ethyl-7-oxo-4b,5,6,7,8,8 a,9,10-octahydrophenanthrene-2-carboxylate; compound with (4bS,8aR)-methyl 4b-ethyl-7-oxo-4b,5,6,7,8,8 a,9,10-octahydrophenanthrene-2-carboxylate (94, R²=Ethyl) (7.40 g, 25.8 mmol) and toluene (200 mL) under a nitrogen atmosphere in a flask fitted with a Dean-Stark trap and condenser. The reaction was heated at reflux and water was removed with a Dean-Stark trap for about 18 h. The reaction mixture was cooled to rt and poured into a solution of saturated aqueous NaHCO₃ (100 mL). The layers were separated and the organic layer was washed with saturated aqueous NaCl (75 mL). The organic layer was dried over MgSO₄, filtered, and concentrated under reduced pressure. The residue was purified on silica gel (120 g) using DCM as eluant. The product containing fractions were combined and concentrated under reduced pressure to afford (4a′R,10a′S)-methyl 4a′-ethyl-3′,4′,4a′,9′,10′,10a′-hexahydro-1′H-spiro[[1,3]dioxolane-2,2′-phenanthrene]-7′-carboxylate; compound with (4a′S,10a′R)-methyl 4a′-ethyl-3′,4′,4a′,9′,10′,10a′-hexahydro-1′H-spiro[[1,3]dioxolane-2,2′-phenanthrene]-7′-carboxylate (38, R²=Ethyl) (7.30 g, 85%) as an oil. LC/MS, method 3, Rₜ=2.68 min, MS m/z 331 (M+H)⁺. ¹H NMR (400 MHz, DMSO-d₆) δ 7.74-7.64 (m, 2H), 7.41 (d, J=8.2 Hz, 1H), 3.91-3.74 (m, 4H), 3.82 (s, 3H), 2.87-2.78 (m, 2H), 2.27-2.16 (m, 1H), 2.12-1.96 (m, 2H), 1.73-1.42 (m, 6H), 1.34-1.24 (m, 1H), 1.22-1.10 (m, 1H), 0.73 (t, J=7.5 Hz, 3H).

### Step #7: (4a′R,10a′R)-Methyl 4a′-ethyl-3′,4′,4a′,10a′-tetrahydro-1′H-spiro[[1,3]dioxolane-2,2′-phenanthrene]-7′-carboxylate; compound with (4a′S,10a′S)-methyl 4a′-ethyl-3′,4′,4a′,10a′-tetrahydro-1′H-spiro[[1,3]dioxolane-2,2′-phenanthrene]-7′-carboxylate (98, R²=Ethyl)

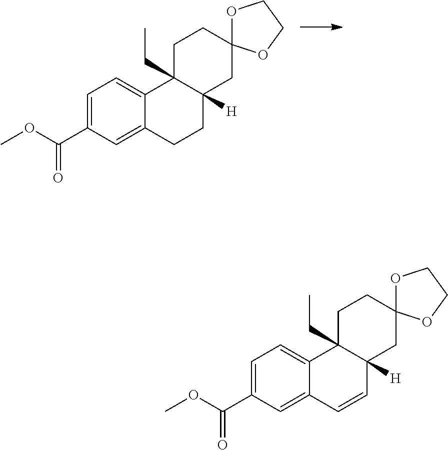

A solution of (4a′R,10a′S)-methyl 4a′-ethyl-3′,4′,4a′,9′,10′,10a′-hexahydro-1′H-spiro[[1,3]dioxolane-2,2′-phenanthrene]-7′-carboxylate; compound with (4a′S,10a′R)-methyl 4a′-ethyl-3′,4′,4a′,9′,10′,10a′-hexahydro-1′H-spiro[[1,3]dioxolane-2,2′-phenanthrene]-7′-carboxylate (38, R²=Ethyl) (3.50 g, 10.6 mmol), N-bromosuccinimide (2.26 g, 12.7 mmol), 2,2′-azobis(2-methylpropionitrile) (0.174 g, 1.059 mmol) and CCl₄ (70 mL) under a nitrogen atmosphere was heated to reflux for about 1 h. The reaction was cooled and diluted with DCM (200 mL), washed with saturated aqueous NaHCO₃ (150 mL), water (50 mL) and saturated aqueous NaCl (100 mL).

The organic layer was dried over MgSO₄, filtered and concentrated under reduced pressure. The residue was dissolved in MeCN (100 mL) and TEA (1.60 mL, 11.6 mmol) was added. The solution was warmed to about 80° C. for about 19 h. The volatiles were removed under reduced pressure. The residue was partitioned between EtOAc (200 mL) and water (100 mL). The aqueous layer was extracted with EtOAc (100 mL). The combined organics were washed with saturated aqueous NaCl (100 mL), dried over MgSO₄, filtered and concentrated under reduced pressure. The residue was purified on silica gel (80 g) using a gradient of 3-9% EtOAc in heptane. The product containing fractions were combined and concentrated under reduced pressure to afford (4a′R, 10a′R)-methyl 4a′-ethyl-3′,4′,4a, 10a′-tetrahydro-1′H-spiro[[1,3]dioxolane-2,2′-phenanthrene]-7′-carboxylate; compound with (4a′S,10a′S)-methyl 4a′-ethyl-3′,4′,4a, 10a′-tetrahydro-1′H-spiro[[1,3]dioxolane-2,2′-phenanthrene]-7′-carboxylate (98, R²=Ethyl) (1.90 g, 55%) as an oil. LC/MS, method 3, Rₜ=2.66 min, MS m/z 329 (M+H)⁺. ¹H NMR (400 MHz, CDCl₃) δ 7.86 (dd, J=8.0, 1.9 Hz, 1H), 7.72 (d, J=2.0 Hz, 1H), 7.28 (d, J=8.0 Hz, 1H), 6.43 (d, J=9.6 Hz, 1H), 5.98 (dd, J=9.5, 6.3 Hz, 1H), 3.98-3.84 (m, 4H), 3.91 (s, 3H), 2.49-2.36 (m, 2H), 1.86-1.74 (m, 1H), 1.76-1.60 (m, 4H), 1.34-1.25 (m, 1H), 1.25-1.15 (m, 1H), 0.64 (t, J=7.5 Hz, 3H).

### Step #8: (4bR,8aR)-Methyl 4b-ethyl-7-oxo-4b,5,6,7,8,8a-hexahydrophenanthrene]-2-carboxylate; compound with (4bS,8aS)-methyl 4b-ethyl-7-oxo-4b,5,6,7,8,8a-hexahydrophenanthrene]-2-carboxylate (98A, R²=Ethyl)

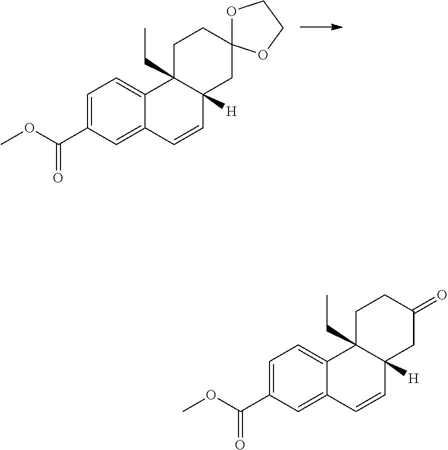

Tfa (1.9 mL, 24 mmol) was added to a biphasic solution of (4a′R,10a′R)-methyl 4a′-ethyl-3′,4′,4a′,10a′-tetrahydro-1′H-spiro[[1,3]dioxolane-2,2′-phenanthrene]-7′-carboxylate; compound with (4a′S,10a′S)-methyl 4a′-ethyl-3′,4′,4a′,10a′-tetrahydro-1′H-spiro[[1,3]dioxolane-2,2′-phenanthrene]-7′-carboxylate (98, R²=Ethyl) (1.60 g, 4.87 mmol), DCM (28 mL), and water (14 mL) under air. The mixture was left to vigorously stir for about 2 h at about 40° C. Tfa (1.0 mL, 13 mmol) was added. The biphasic mixture was left to vigorously stir for about 16 h at about 40° C. TFA (1.0 mL, 13 mmol) was added. The biphasic mixture was left to vigorously stir for about 2 h at about 40° C. The reaction was cooled to rt. DCM (200 mL) was added. The layers were separated and the organics were washed with saturated aqueous NaHCO₃ (150 mL) and saturated aqueous NaCl (150 mL). The organic layer was dried over MgSO₄, filtered, and concentrated under reduced pressure. The residue was purified on silica gel (80 g) using a gradient of 5-17% EtOAc in heptane. The fractions containing product were combined and concentrated under reduced pressure to afford (4bR,8aR)-methyl 4b-ethyl-7-oxo-4b,5,6,7,8,8a-hexahydrophenanthrene-2-carboxylate; compound with (4bS,8aS)-methyl 4b-ethyl-7-oxo-4b,5,6,7,8,8a-hexahydrophenanthrene-2-carboxylate (98A, R²=Ethyl) (1.20 g, 87%) as an ivory foam. LC/MS, method 3, Rₜ=2.39 min, MS m/z 285 (M+H)⁺. ¹H NMR (400 MHz, CDCl₃) δ 7.95 (dd, J=8.0, 1.9 Hz, 1H), 7.80 (d, J=1.8 Hz, 1H), 7.37 (d, J=8.0 Hz, 1H), 6.51 (d, J=9.5 Hz, 1H), 6.00 (dd, J=9.5, 6.0 Hz, 1H), 3.93 (s, 3H), 2.75-2.67 (m, 1H), 2.67-2.59 (m, 1H), 2.54-2.43 (m, 1H), 2.39-2.29 (m, 2H), 2.07-1.98 (m, 1H), 1.96-1.78 (m, 2H), 1.43-1.32 (m, 1H), 0.70 (t, J=7.5 Hz, 3H).

### Step #9: (4bR,7R,8aR)-Methyl 4b-ethyl-7-propyl-7-hydroxy-4b,5,6,7,8,8a-hexahydrophenanthrene-2-carboxylate; compound with (4bS,7S,8aS)-methyl 4b-ethyl-7-propyl-7-hydroxy-4b,5,6,7,8,8a-hexahydrophenanthrene-2-carboxylate (106, R²=Ethyl, R³=Propyl)

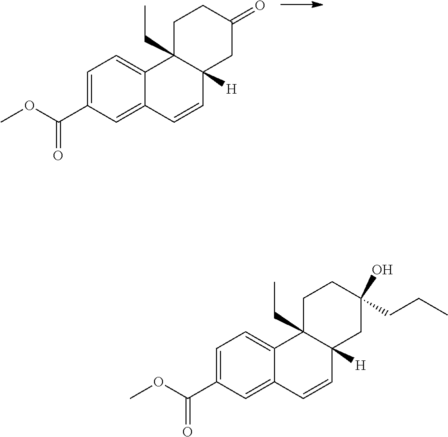

Propylmagnesium bromide (2 M solution in THF, 10.6 mL, 21.2 mmol) [TCI] was added to THF (5 mL) under a nitrogen atmosphere. The solution was cooled to about −45° C. A solution of (4bR,8aR)-methyl 4b-ethyl-7-oxo-4b,5,6,7,8,8a-hexahydrophenanthrene-2-carboxylate; compound with (4bS,8aS)-methyl 4b-ethyl-7-oxo-4b,5,6,7,8,8a-hexahydrophenanthrene-2-carboxylate (98A, R²=Ethyl) (0.600 g, 2.11 mmol) and THF (15 mL) was added dropwise maintaining an internal temperature of less than −40° C. The cold bath was allowed to warm to between −30 and −40° C. over about 15 min and then maintained in this range for about 60 min. MeOH (4 mL) was added dropwise maintaining an internal temperature of less than −10° C. The cold bath was removed and saturated aqueous NH₄Cl (150 mL), water (50 mL) and EtOAc (200 mL) were added. The layers were separated and the organics were washed with saturated aqueous NaCl (50 mL), dried over MgSO₄, filtered, and concentrated under reduced pressure. The residue was purified on silica gel (80 g) using a gradient of 3-30% EtOAc in heptane. The fractions containing product were combined and concentrated under reduced pressure to afford (4bR,7R,8aR)-methyl 4b-ethyl-7-propyl-7-hydroxy-4b,5,6,7,8,8a-hexahydrophenanthrene-2-carboxylate; compound with (4bS,7S,8aS)-methyl 4b-ethyl-7-propyl-7-hydroxy-4b,5,6,7,8,8a-hexahydrophenanthrene-2-carboxylate (106, R²=Ethyl, R³=Propyl) (0.462 g, 53%) as an oil. LC/MS, method 3, Rₜ=2.67 min, MS m/z 329 (M+H)⁺. ¹H NMR (400 MHz, CDCl₃) δ 7.86 (dd, J=8.0, 1.9 Hz, 1H), 7.70 (d, J=1.8 Hz, 1H), 7.27 (d, J=8.0 Hz, 1H), 6.42 (d, J=9.5 Hz, 1H), 6.00 (dd, J=9.5, 6.3 Hz, 1H), 3.90 (s, 3H), 2.56-2.48 (m, 1H), 2.29-2.20 (m, 1H), 1.85-1.70 (m, 3H), 1.60-1.50 (m, 2H), 1.45-1.05 (m, 6H), 1.00-0.80 (m, 4H), 0.64 (t, J=7.6 Hz, 3H).

### Step #10: (4bR,7R,8 aR)-4b-Ethyl-7-propyl-7-hydroxy-N-(2-methylpyridin-3-yl)-4b,5,6,7,8,8 a-hexahydrophenanthrene-2-carboxamide; compound with (4bS,7S,8aS)-4b-ethyl-7-propyl-7-hydroxy-N-(2-methylpyridin-3-yl)-4b,5,6,7,8,8a-hexahydrophenanthrene-2-carboxamide (107, R²=Ethyl, R³=Propyl, R⁶=2-Methylpyridin-3-yl)

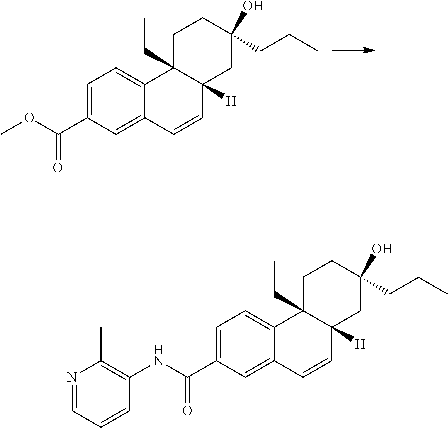

2-Methylpyridin-3-amine (0.183 g, 1.67 mmol) was added in one portion to a solution of (4bR,7R,8aR)-methyl 4b-ethyl-7-propyl-7-hydroxy-4b,5,6,7,8,8a-hexahydrophenanthrene-2-carboxylate; compound with (4bS,7S,8aS)-methyl 4b-ethyl-7-propyl-7-hydroxy-4b,5,6,7,8,8a-hexahydrophenanthrene-2-carboxylate (106, R²=Ethyl, R³=Propyl) (0.462 g, 1.41 mmol) and toluene (10 mL) under a nitrogen atmosphere. The mixture was cooled to about 0° C. LiHMDS (1 M solution in THF, 7.0 mL, 7.0 mmol) was added dropwise over about 30 min. After about 30 min, the ice bath was removed and the mixture was allowed to warm to rt. After about 1 h, the mixture was poured into saturated aqueous NaHCO₃ (20 mL) and water (20 mL). The mixture was extracted with EtOAc (200 mL). The organic layer was washed with water (40 mL), dried over MgSO₄, filtered, and concentrated under reduced pressure. The residue was purified on silica gel (120 g) using a gradient of 0-85% EtOAc in DCM. The fractions containing product were combined and concentrated under reduced pressure to afford (4bR,7R,8aR)-4b-ethyl-7-propyl-7-hydroxy-N-(2-methylpyridin-3-yl)-4b,5,6,7,8,8a-hexahydrophenanthrene-2-carboxamide; compound with (4bS,7S,8aS)-4b-ethyl-7-propyl-7-hydroxy-N-(2-methylpyridin-3-yl)-4b,5,6,7,8,8a-hexahydrophenanthrene-2-carboxamide (107, R²=Ethyl, R³=Propyl, R⁶=2-Methylpyridin-3-yl) (0.440 g, 74%) as a foam. LC/MS, method 2, Rₜ=2.15 min, MS m/z 405 (M+H)⁺. ¹H NMR (400 MHz, CDCl₃) δ 8.43 (d, J=8.0 Hz, 1H), 8.33 (dd, J=4.8, 1.5 Hz, 1H), 7.73-7.66 (m, 2H), 7.55 (d, J=2.0 Hz, 1H), 7.35 (d, J=8.0 Hz, 1H), 7.29-7.23 (m, 1H), 6.46 (d, J=9.5 Hz, 1H), 6.03 (dd, J=9.5, 6.3 Hz, 1H), 2.64 (s, 3H), 2.61-2.53 (m, 1H), 2.31-2.22 (m, 1H), 1.89-1.76 (m, 2H), 1.62-1.53 (m, 2H), 1.48-1.25 (m, 6H), 1.06-1.02 (m, 1H), 1.00-0.89 (m, 1H), 0.89-0.80 (m, 3H), 0.67 (t, J=7.5 Hz, 3H).

### Step #11: (7aS,9R,11aR)-11a-Ethyl-9-propyl-7,9-dihydroxy-N-(2-methylpyridin-3-yl)-5,7,7a,8,9,10,11,11a-octahydrodibenzo[c,e]oxepine-3-carboxamide; compound with (7aR,9S,11aS)-11a-ethyl-9-propyl-7,9-dihydroxy-N-(2-methylpyridin-3-yl)-5,7,7a,8,9,10,11,11a-octahydrodibenzo[c,e]oxepine-3-carboxamide (108, R²=Ethyl, R³=Propyl, R⁶=2-Methylpyridin-3-yl)

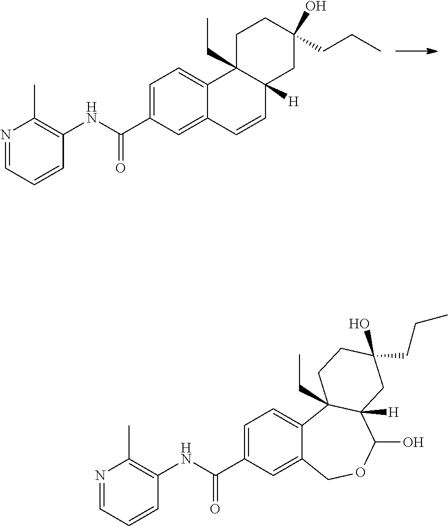

A solution of (4bR,7R,8aR)-4b-ethyl-7-propyl-7-hydroxy-N-(2-methylpyridin-3-yl)-4b,5,6,7,8,8a-hexahydrophenanthrene-2-carboxamide; compound with (4bS,7S,8aS)-4b-ethyl-7-propyl-7-hydroxy-N-(2-methylpyridin-3-yl)-4b,5,6,7,8,8a-hexahydrophenanthrene-2-carboxamide (107, R²=Ethyl, R³=Propyl, R⁶=2-Methylpyridin-3-yl) (0.380 g, 0.939 mmol), DCM (36 mL), and MeOH (4 mL) was purged with O₂ at about −78° C. Ozone was bubbled through the solution (˜2.0 SLPM). After about 8 min, the solution began to turn slightly blue. The ozone generator was switched off and the solution was purged with O₂ for about 30 min. PS-PPh₃ (˜3 mmol/g, 0.94 g) was added. The cold bath was allowed to warm to rt over about 15 min. After about 30 min, the mixture was filtered rinsing with a solution of MeOH (40 mL) and DCM (20 mL). NaBH₄ (0.142 g, 3.76 mmol) was added. After about 30 min, NaBH₄ (0.142 g, 3.76 mmol) was added. After about 30 min, the volatiles were removed under reduced pressure. DCM (50 mL), saturated aqueous NaHCO₃ (20 mL) and water (30 mL) were added. The mixture was left to vigorously stir for about 18 h. The layers were separated and the aqueous layer was extracted with 5% MeOH in DCM (2×20 mL). The combined organics were washed with saturated aqueous NH₄Cl (25 mL). The organic layer was dried over Na₂SO₄, filtered, and concentrated under reduced pressure. The residue was purified on silica gel (40 g) using a gradient of 2-9% MeOH in DCM. The fractions containing product were combined and concentrated under reduced pressure to afford (7aS,9R,11aR)-11a-ethyl-9-propyl-7,9-dihydroxy-N-(2-methylpyridin-3-yl)-5,7,7a,8,9,10,11,11a-octahydrodibenzo[c,e]oxepine-3-carboxamide; compound with (7aR,9S,11aS)-11a-ethyl-9-propyl-7,9-dihydroxy-N-(2-methylpyridin-3-yl)-5,7,7a,8,9,10,11,11a-octahydrodibenzo[c,e]oxepine-3-carboxamide (108, R²=Ethyl, R³=Propyl, R⁶=2-Methylpyridin-3-yl), (0.296 g, 71%) as an ivory solid. LC/MS, method 2, Rₜ=1.62 min, MS m/z 439 (M+H)⁺, ¹H NMR (400 MHz, DMSO-d₆) δ 9.98 (s, 1H), 8.33 (dd, J=4.7, 1.5 Hz, 1H), 7.84 (dd, J=8.3, 1.9 Hz, 1H), 7.79-7.60 (m, 2H), 7.44 (d, J=8.5 Hz, 1H), 7.27 (dd, J=7.9, 4.7 Hz, 1H), 6.44 (d, J=4.3 Hz, 1H), 5.33-5.28 (m, 1H), 4.80-4.63 (m, 2H), 3.83 (s, 1H), 2.44 (s, 3H), 2.35-2.18 (m, 2H), 1.91-1.71 (m, 3H), 1.68-1.58 (m, 1H), 1.50-1.40 (m, 1H), 1.35-1.05 (m, 5H), 0.82-0.62 (m, 7H).

### Step #12: (7aS,9R,11aR)-11a-Ethyl-9-propyl-9-hydroxy-N-(2-methylpyridin-3-yl)-5,7,7a,8,9,10,11,11a-octahydrodibenzo[c,e]oxepine-3-carboxamide; compound with (7aR,9S,11aS)-11a-ethyl-9-propyl-9-hydroxy-N-(2-methylpyridin-3-yl)-5,7,7a,8,9,10,11,11a-octahydrodibenzo[c,e]oxepine-3-carboxamide (110, R²=Ethyl, R³=Propyl, R⁶=2-Methylpyridin-3-yl)

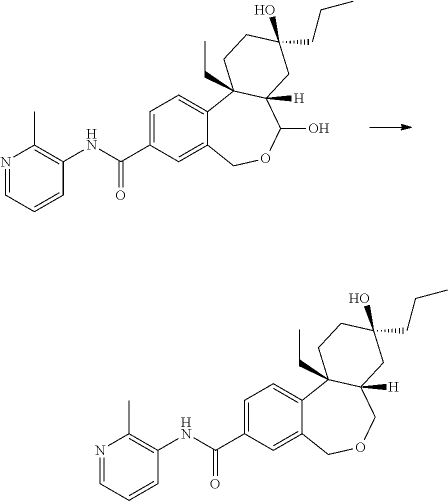

Tfa (0.42 mL, 5.5 mmol) was added to a solution of (7aS,9R,11aR)-11a-ethyl-9-propyl-7,9-dihydroxy-N-(2-methylpyridin-3-yl)-5,7,7a,8,9,10,11,11a-octahydrodibenzo[c,e]oxepine-3-carboxamide; compound with (7aR,9S,11aS)-11a-ethyl-9-propyl-7,9-dihydroxy-N-(2-methylpyridin-3-yl)-5,7,7a,8,9,10,11,11a-octahydrodibenzo[c,e]oxepine-3-carboxamide (108, R²=Ethyl, R³=Propyl, R⁶=2-Methylpyridin-3-yl) (0.294 g, 0.670 mmol) and DCM (6 mL) under a nitrogen atmosphere at rt. Triethylsilane (0.66 mL, 4.1 mmol) was added dropwise. The solution was left to stir for about 16 h. The solution was poured into saturated aqueous NaHCO₃ (30 mL) and then extracted with DCM (50 mL then 2×20 mL). The combined organics were washed with saturated aqueous NaCl (25 mL), dried over Na₂SO₄, filtered, and concentrated under reduced pressure. The residue was purified on silica gel (40 g) using a gradient of 1-5% MeOH in DCM. The fractions containing product were combined and concentrated under reduced pressure to afford (7aS,9R,11aR)-11a-ethyl-9-propyl-9-hydroxy-N-(2-methylpyridin-3-yl)-5,7,7a,8,9,10,11,11a-octahydrodibenzo[c,e]oxepine-3-carboxamide; compound with (7aR,9S,11aS)-11a-ethyl-9-propyl-9-hydroxy-N-(2-methylpyridin-3-yl)-5,7,7a,8,9,10,11,11a-octahydrodibenzo[c,e]oxepine-3-carboxamide (110, R²=Ethyl, R³=Propyl, R⁶=2-Methylpyridin-3-yl) (0.161 g, 56%) as a white solid. LC/MS, method 2, Rₜ=1.85 min, MS m/z 423 (M+H)⁺. ¹H NMR (400 MHz, DMSO-d₆) δ 9.99 (s, 1H), 8.33 (dd, J=4.8, 1.6 Hz, 1H), 7.84 (dd, J=8.2, 1.9 Hz, 1H), 7.76 (d, J=2.0 Hz, 1H), 7.73 (dd, J=8.0, 1.5 Hz, 1H), 7.43 (d, J=8.4 Hz, 1H), 7.27 (dd, J=7.9, 4.7 Hz, 1H), 4.80 (d, J=14.3 Hz, 1H), 4.70 (d, J=14.4 Hz, 1H), 4.25-4.17 (m, 1H), 3.95 (s, 1H), 3.70-3.61 (m, 1H), 2.44 (s, 3H), 2.33-2.23 (m, 1H), 2.12-1.90 (m, 2H), 1.78-1.65 (m, 1H), 1.62-1.45 (m, 2H), 1.42-1.08 (m, 7H), 0.77 (t, J=7.0 Hz, 3H), 0.67 (t, J=7.4 Hz, 3H). Chiral analysis, analytical chiral chromatography method C, UV trace (230-420 nm): Peak 1: Rₜ=6.14 min, 13% of integrated area. Peak 2: Rₜ=6.91 min, 87% of integrated area.

### Example #137

### (7aR,9S,11aS)-11a-ethyl-9-propyl-9-hydroxy-N-(2-methylpyridin-3-yl)-5,7,7a,8,9,10,11,11a-octahydrodibenzo[c,e]oxepine-3-carboxamide (110, R²=Ethyl, R³=Propyl, R⁶=2-Methylpyridin-2-yl) and Example #138: (7aS,9R,11aR)-11a-ethyl-9-propyl-9-hydroxy-N-(2-methylpyridin-3-yl)-5,7,7a,8,9,10,11,11a-octahydrodibenzo[c,e]oxepine-3-carboxamide (110, R²=Ethyl, R³=Propyl, R⁶=2-Methylpyridin-2-yl) Chiral separation of (110, R²=Ethyl, R³=Propyl, R⁶=2-Methylpyridin-3-yl)

Purification Method: (LC) Isocratic, 25% EtOH in heptane with 0.12% diethylamine modifier for 17 min (20 mL/min flow rate). The column used for the chromatography was a 20×250 mm Daicel IA (5 μm particles). The first peak eluted was (7aR,9S,11aS)-11a-ethyl-9-propyl-9-hydroxy-N-(2-methylpyridin-3-yl)-5,7,7a,8,9,10,11,11a-octahydrodibenzo[c,e]oxepine-3-carboxamide (Example 137) and the second was (7aS,9R,11aR)-11a-ethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide (Example 138). NMR and LCMS data for single isomers was essentially identical to the racemic mixture.

Additional examples, prepared in a manner similar to the preparation of Example 137 and Example 138, are listed in Table 8.

TABLE 8

| | | | | | | Chiral |
|---|---|---|---|---|---|---|
| | | | | LC/ | | method/ |
| | | Grignard | | MS | LC/MS | Order of |
| Ex. # | Ketone | Rgt. | Product | method | R_{T}/MH⁺ | elution |
| 139 | Compound | Isobutylmagnesium- | Compound 110 | 2 | 1.98 min | 17/ |
| | 98A (R² = | bromide | (7aR,9S,11aR) | | 437 | Second |
| | Ethyl) | [TCI] | (R² = Ethyl, R³ = | | | |
| | | | Isobutyl, R⁶ = 2- | | | |
| | | | Methylpyridin-3- | | | |
| | | | yl) | | | |
| 140 | Compound | Ethylmagnesium- | Compound 110 | 2 | 1.69 min | 18/ |
| | 98A (R² = | bromide | (7aS,9R,11aR) | | 409 | Second |
| | Ethyl) | | (R² = Ethyl, R³ = | | | |
| | | | Ethyl, R⁶ = 2- | | | |
| | | | Methylpyridin-3- | | | |
| | | | yl) | | | |
| 141 | Compound | Ethylmagnesium- | Compound 110 | 2 | 1.69 min | 18/First |
| | 98A (R² = | bromide | (7aR,9S,11aS) | | 409 | |
| | Ethyl) | | (R² = Ethyl, R³ = | | | |
| | | | Ethyl, R⁶ = 2- | | | |
| | | | Methylpyridin-3- | | | |
| | | | yl) | | | |

### Example #142

### (7aS,9R,11aS)-11a-Benzyl-9-ethyl-9-hydroxy-6,7,7a,8,9,10,11,11a-octahydro-5H-dibenzo[a,c]cycloheptene-3-carboxylic acid (3-amino-phenyl)-amide (85, R⁴=Phenyl, R⁵=Methyl, R⁶=3-Aminophenyl)

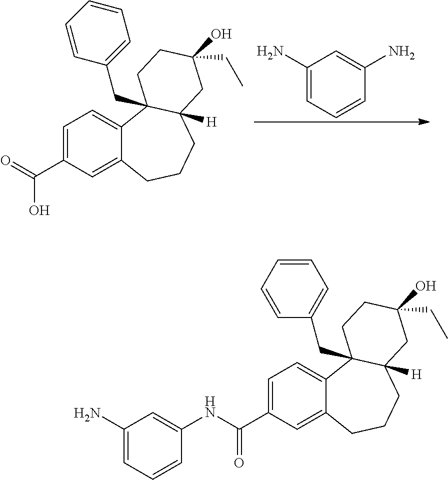

A solution of (7aS,9R,11aS)-11a-benzyl-9-ethyl-9-hydroxy-6,7,7a,8,9,10,11,11a-octahydro-5H-dibenzo[a,c]cycloheptene-3-carboxylic acid (prepared as described in Example 83)(0.044 g, 0.116 mmol) and DIEA (0.030 mL, 0.174 mmol) in DMF (2 mL) was cooled to about 0° C. HBTU (0.053 g, 0.139 mmol) was added and the mixture was stirred for about 10 min. Benzene-1,3-diamine (0.038 g, 0.349 mmol) was then added and the mixture was stirred for about 30 min at about 0° C., then warmed to rt for about 3 h. Water (10 mL) was added and the resulting solids were filtered and rinsed with excess water. The residue was purified on silica gel (4 g) with a gradient of 0-5% MeOH in DCM. Fractions containing product were combined and concentrated under reduced pressure. The residue was further purified on silica gel (4 g) with a gradient of 0-5% MeOH in DCM. Fractions containing product were combined and concentrated under reduced pressure. The residue was dissolved in DMF (2 mL) and purified by reverse phase (C18) HPLC using a gradient of 10-100% MeCN in aqueous NH₄OAc (50 mM) to yield (7aS,9R,11aS)-11a-benzyl-9-ethyl-9-hydroxy-6,7,7a,8,9,10,11,11a-octahydro-5H-dibenzo[a,c]cycloheptene-3-carboxylic acid (3-amino-phenyl)-amide (85, R⁴=Phenyl, R⁵=Methyl, R⁶=3-Aminophenyl) (0.016 g, 29%); LC/MS method 2, Rₜ=2.37 min, MS m/z 469 (M+H)⁺; ¹H NMR (400 MHz, DMSO-d₆) ¹H NMR (400 MHz, DMSO) δ 9.84 (s, 1H), 7.77 (d, J=2.1 Hz, 1H), 7.52 (dd, J=8.2, 2.1 Hz, 1H), 7.17-7.02 (m, 4H), 6.97 (t, J=7.9 Hz, 1H), 6.91-6.84 (m, 1H), 6.80 (d, J=8.4 Hz, 1H), 6.62-6.56 (m, 2H), 6.32 (dd, J=7.9, 2.1 Hz, 1H), 5.07 (bs, 2H), 3.90 (s, 1H), 3.63-3.56 (m, 1H), 3.31-3.24 (m, 1H), 3.09-2.99 (m, 1H), 2.65-2.58 (m, 1H), 2.49-2.41 (m, 1H), 1.91-1.25 (m, 8H), 1.24-1.05 (m, 4H), 0.72 (t, J=7.3 Hz, 3H).

### Example #143

### (7aS,9R,11aS)-11a-Benzyl-9-ethyl-9-hydroxy-6,7,7a,8,9,10,11,11a-octahydro-5H-dibenzo[a,c]cycloheptene-3-carboxylic acid (4-amino-phenyl)-amide (85, R⁴=Phenyl, R⁵=Methyl, R⁶=4-Aminophenyl)

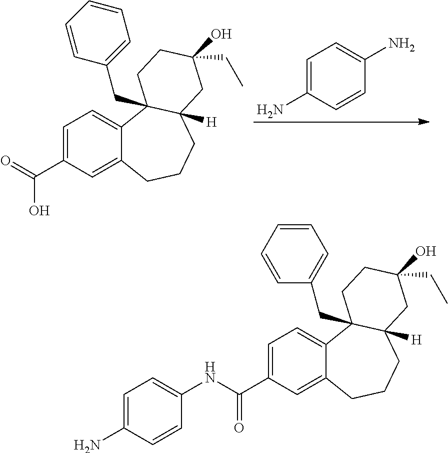

A solution of (7aS,9R,11aS)-11a-benzyl-9-ethyl-9-hydroxy-6,7,7a,8,9,10,11,11a-octahydro-5H-dibenzo[a,c]cycloheptene-3-carboxylic acid (prepared as described in Example 83) (0.044 g, 0.116 mmol) and DIEA (0.030 mL, 0.174 mmol) in DMF (2 mL) was cooled to about 0° C. HBTU (0.053 g, 0.139 mmol) was added and the mixture was stirred for about 10 min. The mixture was cooled to about 0° C., benzene-1,4-diamine (0.038 g, 0.349 mmol) was then added and the mixture was stirred for about 30 min at about 0° C., then warmed to rt for about 2 h. Water (10 mL) was added and the resulting solids were filtered and rinsed with excess water. The residue was purified on silica gel (4 g) with a gradient of 0-5% MeOH in DCM. Fractions containing product were combined and concentrated under reduced pressure. The residue was purified a second time on silica gel (4 g) using a gradient of 0-5% MeOH in DCM. Fractions containing product were combined and concentrated under reduced pressure. The residue was then taken into DMF (2 mL) and purified by reverse phase HPLC using a gradient of 10-100% MeCN in aqueous NH₄OAc (50 nM) to provide (7aS,9R,11aS)-11a-benzyl-9-ethyl-9-hydroxy-6,7,7a,8,9,10,11,11a-octahydro-5H-dibenzo[a,c]cycloheptene-3-carboxylic acid (4-amino-phenyl)-amide (85, R⁴=Phenyl, R⁵=Methyl, R⁶=4-Aminophenyl) (0.040 g, 73%); LC/MS method 2, Rₜ=2.31 min, MS m/z 469 (M+H)⁺; ¹H NMR (400 MHz, DMSO-d₆) δ 9.75 (s, 1H), 7.74 (d, J=2.1 Hz, 1H), 7.48 (dd, J=8.2, 2.1 Hz, 1H), 7.37-7.32 (m, 2H), 7.12-6.99 (m, 3H), 6.76 (d, J=8.4 Hz, 1H), 6.64-6.46 (m, 4H), 4.89 (bs, 2H), 3.88 (s, 1H), 3.57 (d, J=12.9 Hz, 1H), 3.29-3.21 (m, 1H), 3.05-2.96 (m, 1H), 2.61-2.55 (m, 1H), 2.46-2.39 (m, 1H), 1.97-1.68 (m, 3H), 1.70-1.18 (m, 5H), 1.20-1.00 (m, 4H), 0.70 (t, J=7.4 Hz, 3H).

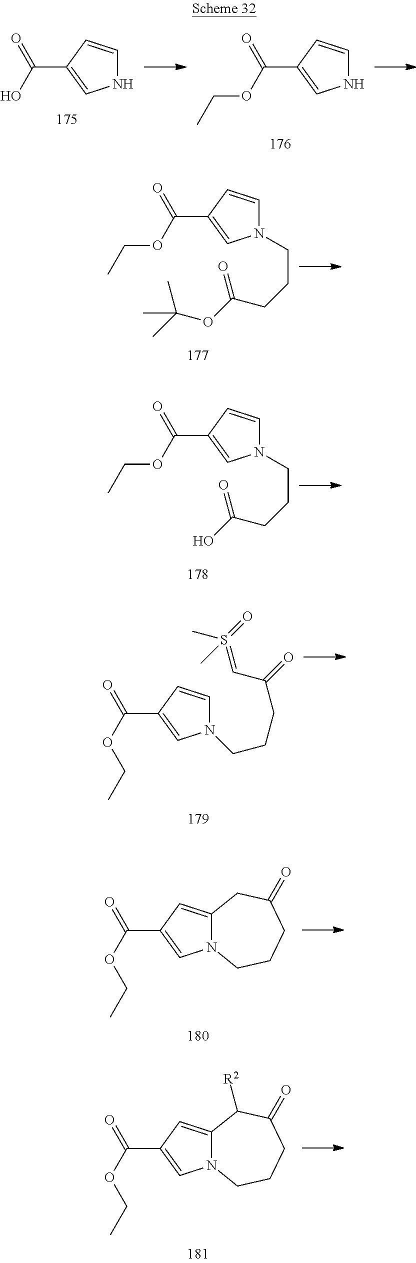

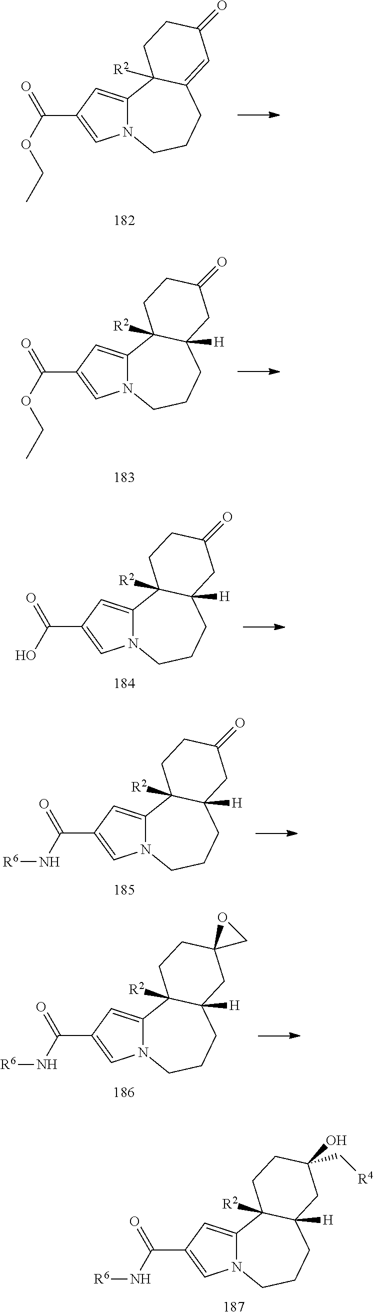

### Example #144

### (7aS,9R,11aS)-11a-Benzyl-9-ethyl-9-hydroxy-N-(2-methylpyridin-3-yl)-6,7,7a,8,9,10,11,11a-octahydro-5H-benzo[c]pyrrolo[1,2-a]azepine-2-carboxamide; compound with (7aR,9S,11aR)-11a-benzyl-9-ethyl-9-hydroxy-N-(2-methylpyridin-3-yl)-6,7,7a,8,9,10,11,11a-octahydro-5H-benzo[c]pyrrolo[1,2-a]azepine-2-carboxamide (187, R²=Benzyl, R⁴=Methyl, R⁶=2-Methylpyridin-3-yl)

### Step #1: Ethyl 1H-pyrrole-3-carboxylate (176)

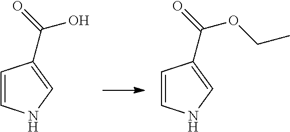

A solution of 1H-pyrrole-3-carboxylic acid (175) (10 g, 90 mmol) in EtOH (450 mL) was treated with H₂SO₄ (0.48 mL, 9.0 mmol) and the resulting solution was stirred at reflux for about 3 days. The reaction mixture was then concentrated under reduced pressure and the residue was then partitioned between saturated aqueous NaHCO₃ (250 mL) and EtOAc (250 mL). After separating the layers, the organic phase was washed with saturated aqueous NaCl (200 mL), dried over Na₂SO₄, filtered, and concentrated under reduced pressure. The sample was purified on silica gel (220 g) using a gradient of 0-50% EtOAc in heptane to yield ethyl 1H-pyrrole-3-carboxylate (176) (9.2 g, 74%). LC/MS, method 3, Rₜ=1.71 min, MS m/z 140 (M+H)⁺. ¹H NMR (400 MHz, CDCl₃) δ 8.66-8.43 (bs, 1H), 7.45-7.41 (m, 1H), 6.78-6.74 (m, 1H), 6.68-6.64 (m, 1H), 4.29 (q, J=7.1 Hz, 2H), 1.34 (t, J=7.1 Hz, 3H).

### Step #2: Ethyl 1-(4-tert-butoxy-4-oxobutyl)-1H-pyrrole-3-carboxylate (177)

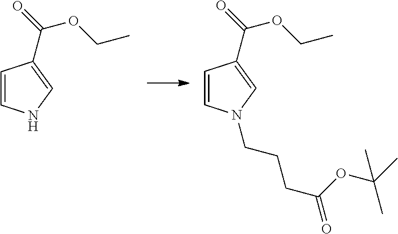

A solution of ethyl 1H-pyrrole-3-carboxylate (176) (7.6 g, 55 mmol) in DMF (273 mL) was cooled in an ice bath and then treated with NaH (60% dispersion in mineral oil; 3.3 g, 82 mmol). Once gas evolution had subsided, the suspension was heated at about 50° C. for about 1 h. tert-Butyl 4-bromobutanoate (14 mL, 82 mmol) was added and stirring was continued at 50° C. for 16 h. The reaction was concentrated under reduced pressure and the residue was partitioned between EtOAc (250 mL) and water (250 mL). After separating the layers, the organic phase was washed with saturated aqueous NaCl (200 mL), dried over Na₂SO₄, filtered, and concentrated under reduced pressure. The crude material was purified on silica gel (330 g) using a gradient of 0-50% EtOAc in heptane to yield ethyl 1-(4-tert-butoxy-4-oxobutyl)-1H-pyrrole-3-carboxylate (177) (11.8 g, 77%). LC/MS, method 3, Rₜ=2.42 min, MS m/z 282 (M+H)⁺. ¹H NMR (400 MHz, CDCl₃) δ 7.27-7.26 (m, 1H), 6.60-6.55 (m, 2H), 4.30-4.19 (m, 2H), 3.93 (t, J=6.8 Hz, 2H), 2.21-2.15 (m, 2H), 2.12-1.98 (m, 2H), 1.45 (s, 9H), 1.36-1.30 (t, J=7.1 Hz, 3H).

### Step #3: 4-(3-(Ethoxycarbonyl)-1H-pyrrol-1-yl)butanoic acid (178)

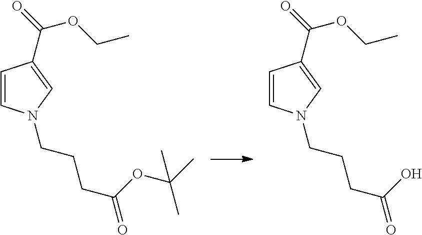

A solution of ethyl 1-(4-tert-butoxy-4-oxobutyl)-1H-pyrrole-3-carboxylate (177) (3.16 g, 11.2 mmol) in DCM (22.5 mL) was treated with Tfa (8.6 mL, 110 mmol) and the solution was stirred at rt for about 2 h. The reaction was then concentrated under reduced pressure, and the residue was re-dissolved in toluene (25 mL). The solution was again concentrated under reduced pressure, re-dissolved in toluene (25 mL) and then finally concentrated to dryness under reduced pressure to afford, without further purification, 4-(3-(ethoxycarbonyl)-1H-pyrrol-1-yl)butanoic acid (178) (2.53 g, 100%). LC/MS, method 3, Rₜ=1.71 min, MS m/z 226 (M+H)⁺. ¹H NMR (400 MHz, CDCl₃) δ 9.40 (bs, 1H), 7.32-7.28 (m, 1H), 6.60-6.58 (m, 2H), 4.27 (q, J=7.1 Hz, 2H), 3.97 (t, J=6.9 Hz, 2H), 2.35 (t, J=7.1 Hz, 2H), 2.15-2.06 (m, 2H), 1.34 (t, J=7.1 Hz, 3H).

Step #4: Compound 179

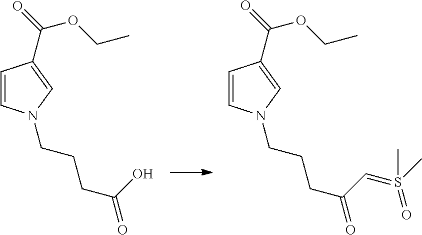

A suspension of 4-(3-(ethoxycarbonyl)-1H-pyrrol-1-yl)butanoic acid (178) (2.53 g, 11.2 mmol) and HATU (4.27 g, 11.2 mmol) in THF (37 mL) was treated with TEA (5.5 mL, 39 mmol) and the resulting solution was stirred at rt for about 16 h. Separately, a suspension of potassium tert-butoxide (3.78 g, 33.7 mmol) and trimethylsulfoxonium chloride (4.33 g, 33.7 mmol) in THF (37 mL) was heated at about 60° C. for about 2 h, and then cooled in an ice-water bath for about 15 min. The solution of activated ester was then added drop-wise at about 0° C. over a period of about 45 min. The reaction mixture was further stirred for about 1 h, after which the reaction was concentrated under reduced pressure. The residue was partitioned between DCM (100 mL) and water (100 mL). After separating the layers, the organic phase was washed with saturated aqueous NaCl (100 mL), dried over Na₂SO₄, filtered, and concentrated under reduced pressure. The crude material was purified on silica gel (80 g) using a gradient of 0-5% MeOH in DCM to yield Compound 179 (2.22 g, 66%). LC/MS, method 3, Rₜ=1.44 min, MS m/z 300 (M+H)⁺. ¹H NMR (400 MHz, CDCl₃) δ 7.29-7.26 (m, 1H), 6.62-6.54 (m, 2H), 4.35 (s, 1H), 4.26 (q, J=7.1 Hz, 2H), 3.92 (t, J=6.8 Hz, 2H), 3.38 (s, 6H), 2.20-2.11 (m, 2H), 2.12-1.99 (m, 2H), 1.33 (t, J=7.1 Hz, 3H).

### Step #5: Ethyl 8-oxo-6,7,8,9-tetrahydro-5H-pyrrolo[1,2-c]azepine-2-carboxylate (180)

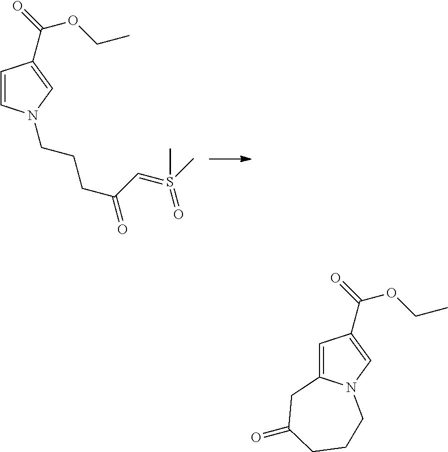

A solution of Compound 179 (2.22 g, 7.42 mmol) and chloro(1,5-cyclooctadiene)iridium(I) dimer (0.498 g, 0.742 mmol) in DCE (297 mL) was degassed with a stream of nitrogen gas for about 30 min. The mixture was heated at about 80° C. for about 10 min, and then cooled to rt. The reaction was concentrated under reduced pressure. The residue was purified on silica gel (80 g) using 10% EtOAc in heptane as eluant to give ethyl 8-oxo-6,7,8,9-tetrahydro-5H-pyrrolo[1,2-c]azepine-2-carboxylate (180) (0.87 g, 53%). LC/MS, method 3, Rₜ=1.72 min, MS m/z 222 (M+H)⁺. ¹H NMR (400 MHz, CDCl₃) δ 7.27-7.25 (m, 1H), 6.44-6.42 (m, 1H), 4.26 (q, J=7.1 Hz, 2H), 4.20-4.13 (m, 2H), 3.68 (s, 2H), 2.59 (t, J=6.8 Hz, 2H), 2.17-2.06 (m, 2H), 1.32 (t, J=7.1 Hz, 3H).

### Step #6: Ethyl 9-benzyl-8-oxo-6,7,8,9-tetrahydro-5H-pyrrolo[1,2-c]azepine-2-carboxylate (181, R²=Benzyl)

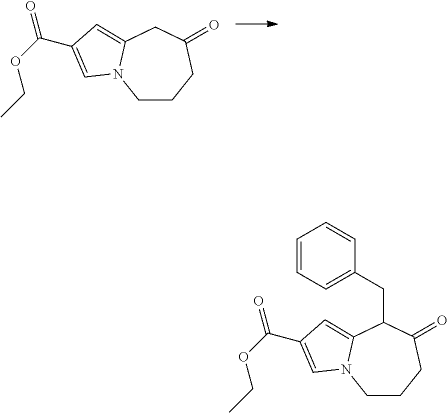

A solution of ethyl 8-oxo-6,7,8,9-tetrahydro-5H-pyrrolo[1,2-a]azepine-2-carboxylate (180) (0.87 g, 3.9 mmol) in toluene (39 mL) was treated with pyrrolidine (0.72 mL, 8.6 mmol) and the reaction mixture was heated at reflux for about 3 h, removing water by means of a Dean-Stark trap. The reaction was cooled and concentrated under reduced pressure, then re-dissolved in 1,4-dioxane (26 mL), treated with benzyl bromide (0.84 mL, 7.1 mmol), and then heated at about 100° C. for about 21 h. The reaction mixture was allowed to cool to rt and then was partitioned between water (150 mL) and EtOAc (150 mL). After separating the layers, the aqueous phase was extracted with EtOAc (50 mL). The combined organic phases were washed with saturated aqueous NaCl (100 mL), dried over Na₂SO₄, filtered, and concentrated under reduced pressure. The crude material was purified on silica gel (330 g) using a gradient of 0-50% EtOAc in heptane to yield ethyl 9-benzyl-8-oxo-6,7,8,9-tetrahydro-5H-pyrrolo[1,2-a]azepine-2-carboxylate (181, R²=Benzyl) (0.69 g, 56%). LC/MS, method 3, Rₜ=2.29 min, MS m/z 229 (M+H)⁺. ¹H NMR (400 MHz, CDCl₃) δ 7.27-7.15 (m, 6H), 6.50-6.46 (m, 1H), 4.26 (q, J=7.1 Hz, 2H), 4.21-4.10 (m, 2H), 4.02-3.90 (m, 1H), 3.46 (dd, J=13.8, 8.6 Hz, 1H), 3.14 (dd, J=13.8, 4.9 Hz, 1H), 2.58-2.50 (m, 2H), 2.24-2.12 (m, 1H), 2.02-1.86 (m, 1H), 1.32 (t, J=7.1 Hz, 3H).

### Step #7: Ethyl 11a-benzyl-9-oxo-6,7,9,10,11,11a-hexahydro-5H-benzo[c]pyrrolo[1,2-a]azepine-2-carboxylate (182, R²=benzyl)

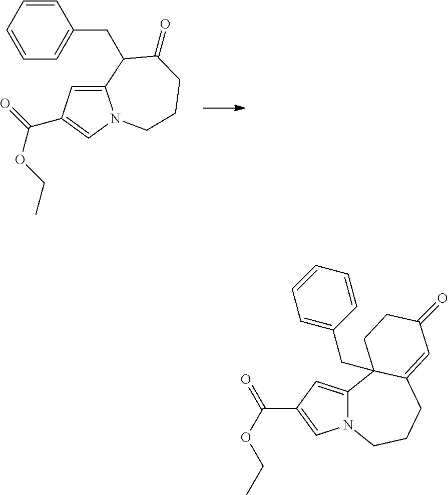

Sodium (0.076 g, 3.3 mmol) was added to a flask containing EtOH (6 mL), and stirred at room temperature until the reaction was complete. A suspension of ethyl 9-benzyl-8-oxo-6,7,8,9-tetrahydro-5H-pyrrolo[1,2-c]azepine-2-carboxylate (181, R²=Benzyl) (0.69 g, 2.2 mmol) in EtOH (6 mL) was added and the mixture was heated at about 60° C. for about 5 min. Methyl vinyl ketone (0.20 ml, 2.4 mmol) was added drop-wise over about 30 min. The reaction was stirred at about 60° C. for about 60 min, then allowed to cool to rt. The reaction mixture was concentrated under reduced pressure and the residue was partitioned between EtOAc (50 mL) and 10% aqueous NH₄Cl (50 mL). After separating the layers, the organic phase was washed with saturated aqueous NaCl (25 mL), dried over Na₂SO₄, filtered, and concentrated under reduced pressure. The sample was purified on silica gel (40 g) using a gradient of 0-50% EtOAc in heptane to give ethyl 11a-benzyl-9-oxo-6,7,9,10,11,11a-hexahydro-5H-benzokipyrrolo[1,2-a]azepine-2-carboxylate (182, R²=Benzyl) (0.311 g, 39%). LC/MS, method 3, Rₜ=2.27 min, MS m/z 364 (M+H)⁺. ¹H NMR (400 MHz, CDCl₃) δ 7.23 (d, J=1.9 Hz, 1H), 7.20-7.12 (m, 3H), 6.87-6.80 (m, 2H), 6.42 (d, J=1.9 Hz, 1H), 5.98 (s, 1H), 4.26 (q, J=7.1 Hz, 2H), 4.22-4.09 (m, 2H), 3.50 (d, J=13.3 Hz, 1H), 2.98 (d, J=13.3 Hz, 1H), 2.66-2.35 (m, 4H), 2.26-2.11 (m, 2H), 2.09-1.98 (m, 1H), 1.87-1.72 (m, 1H), 1.33 (t, J=7.1 Hz, 3H).

### Step #8: (7aS,11aS)-Ethyl 11a-benzyl-9-oxo-6,7,7a,8,9,10,11,11a-octahydro-5H-benzo[c]pyrrolo[1,2-a]azepine-2-carboxylate; compound with (7aR,11aR)— ethyl 11a-benzyl-9-oxo-6,7,7a,8,9,10,11,11a-octahydro-5H-benzo[c]pyrrolo[1,2-a]azepine-2-carboxylate (183, R²=Benzyl)

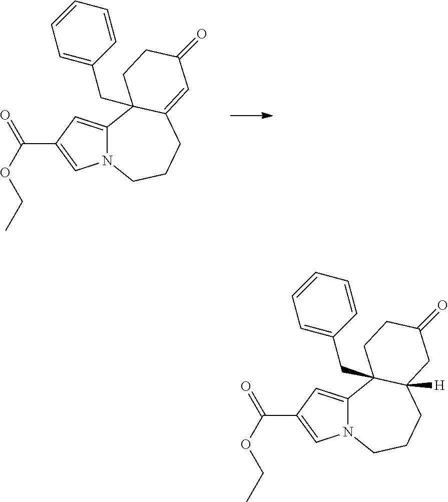

A suspension of ethyl 11a-benzyl-9-oxo-6,7,9,10,11,11a-hexahydro-5H-benzo[c]pyrrolo[1,2-c]azepine-2-carboxylate (182, R²=Benzyl) (0.100 g, 0.275 mmol) and 10% Pd on carbon (0.029 g) in EtOAc (20 mL) was shaken in a Parr Shaker at rt under about 55 psi of hydrogen for about 2 h. The reaction was filtered through a pad of Celite® (about 1.0 g) to remove the catalyst. The Celite® pad was washed with EtOAc (3×5 mL). The filtrates were combined and concentrated under reduced pressure, and then the residue was purified on silica gel (12 g) using a gradient of 10-35% EtOAc in heptane to yield (7aS,11aS)-ethyl 11a-benzyl-9-oxo-6,7,7a,8,9,10,11,11a-octahydro-5H-benzo[c]pyrrolo[1,2-a]azepine-2-carboxylate; compound with (7aR,11aR)-ethyl 11a-benzyl-9-oxo-6,7,7a,8,9,10,11,11a-octahydro-5H-benzo[c]pyrrolo[1,2-a]azepine-2-carboxylate (183, R²=Benzyl) (0.089 g, 89%). LC/MS method 3, Rₜ=2.48 min, MS m/z: 366 (M+H)⁺. ¹H NMR (400 MHz, CDCl₃) δ 7.29 (d, J=1.9 Hz, 1H), 7.21-7.08 (m, 3H), 6.64-6.52 (m, 2H), 6.18 (d, J=1.9 Hz, 1H), 4.33-4.19 (m, 4H), 3.48 (d, J=13.3 Hz, 1H), 2.75-2.61 (m, 1H), 2.53 (d, J=13.3 Hz, 1H), 2.51-2.41 (m, 1H), 2.40-2.21 (m, 3H), 2.19-2.08 (m, 1H), 2.08-1.90 (m, 2H), 1.90-1.74 (m, 2H), 1.74-1.62 (m, 1H), 1.31 (t, J=7.1 Hz, 3H).

### Step #9: (7aS,11aS)-11a-Benzyl-9-oxo-6,7,7a,8,9,10,11,11a-octahydro-5H-benzo[c]pyrrolo[1,2-a]azepine-2-carboxylic acid; compound with (7aR,11aR)-11a-benzyl-9-oxo-6,7,7a,8,9,10,11,11a-octahydro-5H-benzo[c]pyrrolo[1,2-a]azepine-2-carboxylic acid (184, R²=Benzyl)

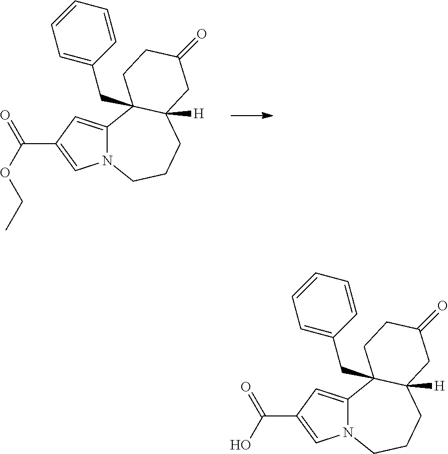

A 10 mL microwave reaction vial was charged with (7aS,11aS)-ethyl 11a-benzyl-9-oxo-6,7,7a,8,9,10,11,11a-octahydro-5H-benzo[c]pyrrolo[1,2-a]azepine-2-carboxylate; compound with (7aR,11aR)-ethyl 11a-benzyl-9-oxo-6,7,7a,8,9,10,11,11a-octahydro-5H-benzo[c]pyrrolo[1,2-a]azepine-2-carboxylate (183, R²=Benzyl) (0.250 g, 0.684 mmol) and LiOH (0.164 g, 6.84 mmol) in 1,4-dioxane (2.5 mL) and water (2.5 mL) and sealed with a pressure releasing septa cap. The reaction mixture was heated in a Biotage microwave at about 120° C. for about 30 minutes (250 psi max pressure, 5 min ramp, 300 max watts). The pH of the reaction mixture was adjusted to about pH=2 by drop-wise addition of aqueous 1N aqueous HCl. The resulting suspension was partitioned between EtOAc (50 mL) and water (50 mL). After separating the layers, the organic phase was washed with saturated aqueous NaCl (50 mL), dried over Na₂SO₄, filtered, and concentrated to give (7aS,11aS)-11a-benzyl-9-oxo-6,7,7a,8,9,10,11,11a-octahydro-5H-benzo[c]pyrrolo[1,2-a]azepine-2-carboxylic acid; compound with (7aR,11aR)-11a-benzyl-9-oxo-6,7,7a,8,9,10,11,11a-octahydro-5H-benzo[c]pyrrolo[1,2-a]azepine-2-carboxylic acid (184, R²=Benzyl) (0.187 g, 81%). LC/MS, method 3, Rₜ=2.03 min, MS m/z 338 (M+H)⁺. ¹H NMR (400 MHz, DMSO-d₆) δ 11.57 (s, 1H), 7.42 (d, J=1.9 Hz, 1H), 7.15-7.07 (m, 3H), 6.61-6.55 (m, 2H), 5.92 (d, J=1.9 Hz, 1H), 4.44-4.21 (m, 2H), 3.51 (d, J=13.1 Hz, 1H), 2.59-2.51 (m, 2H), 2.48-2.35 (m, 1H), 2.30-2.05 (m, 3H), 2.05-1.59 (m, 6H).

### Step #10: (7aS,11aS)-11a-Benzyl-N-(2-methylpyridin-3-yl)-9-oxo-6,7,7a,8,9,10,11,11a-octahydro-5H-benzo[c]pyrrolo[1,2-c]azepine-2-carboxamide; compound with (7aR,11aR)-11a-benzyl-N-(2-methylpyridin-3-yl)-9-oxo-6,7,7a,8,9,10,11,11a-octahydro-5H-benzo[c]pyrrolo[1,2-c]azepine-2-carboxamide (185, R²=Benzyl, R⁶=2-Methylpyridin-3-yl)

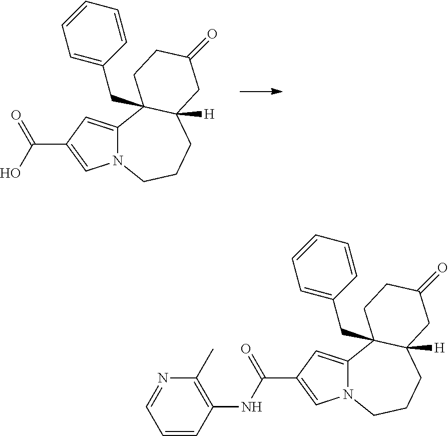

A mixture of 3-amino-2-picoline (0.120 g, 1.11 mmol), (7aS,11aS)-11a-benzyl-9-oxo-6,7,7a,8,9,10,11,11a-octahydro-5H-benzo[c]pyrrolo[1,2-a]azepine-2-carboxylic acid; compound with (7aR,11aR)-11a-benzyl-9-oxo-6,7,7a,8,9,10,11,11a-octahydro-5H-benzo[c]pyrrolo[1,2-a]azepine-2-carboxylic acid (184, R²=Benzyl) (0.187 g, 0.554 mmol), and TFFH (0.146 g, 0.554 mmol) in THF (2.8 mL) was treated with DIEA (0.10 mL, 0.55 mmol) and the resulting suspension was allowed to stir at rt for about 3 days. The reaction mixture was diluted with DCM (25 mL), and the solution was washed with saturated aqueous NaHCO₃ (25 mL). The organic phase was washed with saturated aqueous NaCl (20 mL), dried over Na₂SO₄, filtered, and concentrated under reduced pressure. The residue was purified on silica gel (12 g) using a gradient of 0-5% MeOH in DCM to yield (7aS,11aS)-11a-benzyl-N-(2-methylpyridin-3-yl)-9-oxo-6,7,7a,8,9,10,11,11a-octahydro-5H-benzo[c]pyrrolo[1,2-a]azepine-2-carboxamide; compound with (7aR,11aR)-11a-benzyl-N-(2-methylpyridin-3-yl)-9-oxo-6,7,7a,8,9,10,11,11a-octahydro-5H-benzo[c]pyrrolo[1,2-a]azepine-2-carboxamide (185, R²=Benzyl, R⁶=2-Methylpyridin-3-yl) (0.034 g, 14%). LC/MS, method 2, Rₜ=1.96 min, MS m/z 428 (M+H)⁺. ¹H NMR (400 MHz, DMSO-d₆) δ 9.12 (s, 1H), 8.25 (dd, J=4.7, 1.6 Hz, 1H), 7.67 (dd, J=8.0, 1.6 Hz, 1H), 7.51 (d, J=1.9 Hz, 1H), 7.21 (dd, J=7.9, 4.7 Hz, 1H), 7.17-7.08 (m, 3H), 6.68-6.58 (m, 2H), 6.28 (d, J=2.0 Hz, 1H), 4.41-4.26 (m, 2H), 3.53 (d, J=13.2 Hz, 1H), 2.71-2.42 (m, 3H), 2.37 (s, 3H), 2.31-2.14 (m, 3H), 2.06-1.63 (m, 6H).

### Step #11: (2′R,7aS,11aS)-11a-Benzyl-N-(2-methylpyridin-3-yl)-5,6,7,7a,8,10,11,11a-octahydrospiro[benzo[c]pyrrolo[1,2-a]azepine-9,2′-oxirane]-2-carb oxamide; compound with (2′S,7aR,11aR)-11a-benzyl-N-(2-methylpyridin-3-yl)-5,6,7,7a,8,10,11,11a-octahydrospiro[benzo[c]pyrrolo[1,2-a]azepine-9,2′-oxirane]-2-carboxamide (186, R²=Benzyl, R⁶=2-Methylpyridin-3-yl)

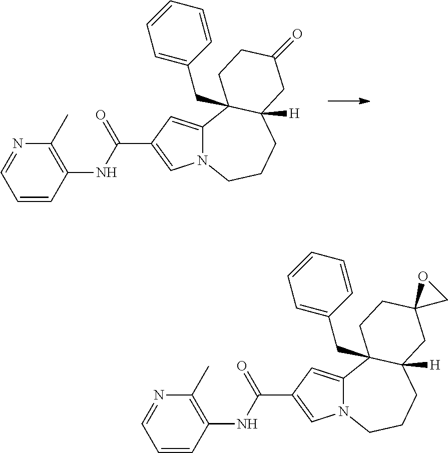

A suspension of NaH (60% dispersion in mineral oil; 5.4 mg, 0.14 mmol) in DMSO (0.34 mL) was heated at about 60° C. for about 30 min. The mixture was cooled to rt and trimethylsulfoxonium iodide (0.030 g, 0.14 mmol) was added in one portion. The resulting solution was allowed to stir at rt for about 15 min. A solution of (7aS,11aS)-11a-benzyl-N-(2-methylpyridin-3-yl)-9-oxo-6,7,7a,8,9,10,11,11a-octahydro-5H-benzo[c]pyrrolo[1,2-a]azepine-2-carboxamide; compound with (7aR,11aR)— 11a-benzyl-N-(2-methylpyridin-3-yl)-9-oxo-6,7,7a,8,9,10,11,11a-octahydro-5H-benzo[c]pyrrolo[1,2-a]azepine-2-carboxamide (185, R²=Benzyl, R⁶=2-Methylpyridin-3-yl) (0.029 g, 0.068 mmol) in THF (0.34 mL) was added in one portion and stirring was continued for about 1.5 h. The reaction mixture was partitioned between EtOAc (10 mL) and water (10 mL). After separating the layers, the organic phase was washed with saturated aqueous NaCl (10 mL), dried over Na₂SO₄, filtered, and concentrated under reduced pressure. The material was purified on silica gel (4 g) using a gradient of 0-5% MeOH in DCM to yield (2′R,7aS,11aS)-11a-benzyl-N-(2-methylpyridin-3-yl)-5,6,7,7a,8,10,11,11a-octahydrospiro[benzo[c]pyrrolo[1,2-a]azepine-9,2′-oxirane]-2-carboxamide; compound with (2′S,7aR,11aR)-11a-benzyl-N-(2-methylpyridin-3-yl)-5,6,7,7a,8,10,11,11a-octahydrospiro[benzo[c]pyrrolo[1,2-a]azepine-9,2′-oxirane]-2-carboxamide (186, R²=Benzyl, R⁶=2-Methylpyridin-3-yl) (0.0166 g, 55%). LC/MS, method 3, Rₜ=2.07 min, MS m/z 442 (M+H)⁺. ¹H NMR (400 MHz, DMSO-d₆) δ 9.11 (s, 1H), 8.25 (dd, J=4.7, 1.6 Hz, 1H), 7.67 (dd, J=8.0, 1.6 Hz, 1H), 7.46 (d, J=1.9 Hz, 1H), 7.20 (dd, J=8.0, 4.7 Hz, 1H), 7.16-7.08 (m, 3H), 6.67-6.61 (m, 2H), 6.20 (d, J=1.9 Hz, 1H), 4.40-4.21 (m, 2H), 3.44 (d, J=13.1 Hz, 1H), 2.62 (d, J=13.2 Hz, 1H), 2.57-2.52 (m, 2H), 2.48-2.40 (m, 1H), 2.37 (s, 3H), 2.36-2.27 (m, 1H), 2.21-2.11 (m, 1H), 1.92-1.77 (m, 3H), 1.73-1.54 (m, 3H), 1.09-0.98 (d, J=14.6 Hz, 1H), 0.81 (d, J=13.7 Hz, 1H).

Step #12: (7aS,9R,11aS)-11a-Benzyl-9-ethyl-9-hydroxy-N-(2-methylpyridin-3-yl)-6,7,7a,8,9,10,11,11a-octahydro-5H-benzo[c]pyrrolo[1,2-a]azepine-2-carboxamide; compound with (7aR,9S,11aR)-11a-benzyl-9-ethyl-9-hydroxy-N-(2-methylpyridin-3-yl)-6,7,7a,8,9,10,11,11a-octahydro-5H-benzo[c]pyrrolo[1,2-a]azepine-2-carboxamide (187, R²=Benzyl, R⁴=Methyl, R⁶=2-Methylpyridin-3-yl)

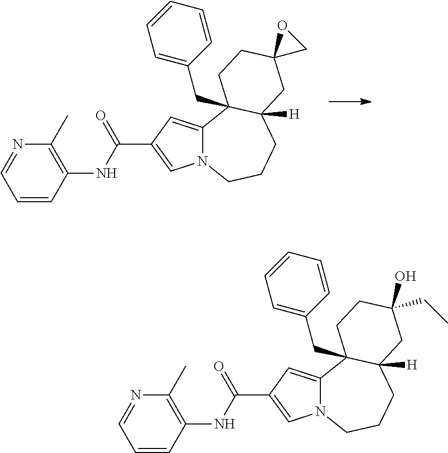

A stirred suspension of CuI (1 mg, 0.005 mmol) and (2′R,7aS,11aS)-11a-benzyl-N-(2-methylpyridin-3-yl)-5,6,7,7a,8,10,11,11a-octahydro spiro[benzo[c]pyrrolo[1,2-a]azepine-9,2′-oxirane]-2-carboxamide; compound with (2′S,7aR,11aR)-11a-benzyl-N-(2-methylpyridin-3-yl)-5,6,7,7a,8,10,11,11a-octahydro spiro[benzo[c]pyrrolo[1,2-a]azepine-9,2′-oxirane]-2-carb oxamide (186, R²=Benzyl, R⁶=2-Methylpyridin-3-yl) (0.016 g, 0.036 mmol) in THF (0.36 mL) was treated at rt with methylmagnesium bromide (3 M solution in Et₂O, 0.072 mL, 0.22 mmol). The reaction mixture was quenched at rt by addition of saturated aqueous NH₄Cl (1 mL), and the resulting mixture was partitioned between water (2 mL) and EtOAc (2 mL). After separating the layers, the aqueous phase was extracted with EtOAc (2×5 mL) and DCM (3×5 mL). The combined organic phases were washed with saturated aqueous NaCl (10 mL), dried over Na₂SO₄, and filtered through a pad of Florisil®. The filtrates were combined and concentrated under reduced pressure to give (7aS,9R,11aS)-11a-benzyl-9-ethyl-9-hydroxy-N-(2-methylpyridin-3-yl)-6,7,7a,8,9,10,11,11a-octahydro-5H-benzo[c]pyrrolo[1,2-a]azepine-2-carboxamide; compound with (7aR,9S,11aR)-11a-benzyl-9-ethyl-9-hydroxy-N-(2-methylpyridin-3-yl)-6,7,7a,8,9,10,11,11a-octahydro-5H-benzo[c]pyrrolo[1,2-a]azepine-2-carboxamide (187, R²=Benzyl, R⁴=Methyl, R⁶=2-Methylpyridin-3-yl) (0.015 g, 90%). LC/MS, method 3, Rₜ=2.00 min, MS m/z 458 (M+H)⁺.

¹H NMR (400 MHz, DMSO-d₆) δ 9.07 (s, 1H), 8.24 (dd, J=4.7, 1.6 Hz, 1H), 7.67 (dd, J=8.0, 1.6 Hz, 1H), 7.42 (d, J=1.9 Hz, 1H), 7.20 (dd, J=7.9, 4.7 Hz, 1H), 7.14-7.06 (m, 3H), 6.62-6.55 (m, 2H), 6.08 (d, J=1.9 Hz, 1H), 4.36-4.17 (m, 2H), 3.82 (s, 1H), 3.39 (d, J=13.0 Hz, 1H), 2.58-2.52 (m, 1H), 2.48-2.26 (m, 6H), 1.91-1.78 (m, 1H), 1.77-1.50 (m, 5H), 1.40-1.31 (m, 1H), 1.29-1.09 (m, 3H), 0.76 (t, J=7.4 Hz, 3H).

### Example #145

### (7aS,9R,11aR)-11a-Ethyl-9-hydroxy-9-propyl-6,7,7a,8,9,10,11,11a-octahydro-5H-dibenzo[a,c]cycloheptene-3-carboxylic acid (2-amino-phenyl)-amide; compound with (7aR,9S,11aS)-11a-ethyl-9-hydroxy-9-propyl-6,7,7a,8,9,10,11,11a-octahydro-5H-dibenzo[a,c]cycloheptene-3-carboxylic acid (2-amino-phenyl)-amide (85, R⁴=Methyl, R⁵=Ethyl, R⁶=2-Amino-phenyl)

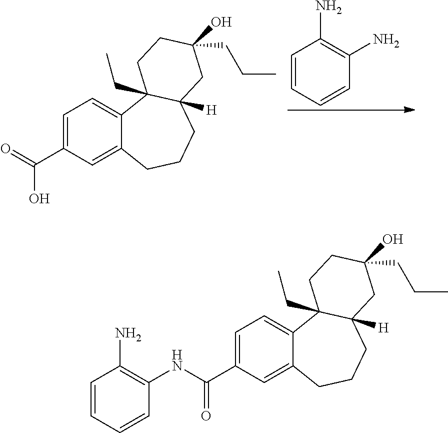

A solution of (7aS,9R,11aR)-11a-ethyl-9-hydroxy-9-propyl-6,7,7a,8,9,10,11,11a-octahydro-5H-dibenzo[a,c]cycloheptene-3-carboxylic acid; compound with (7aR,9S,11aS)-11a-ethyl-9-hydroxy-9-propyl-6,7,7a,8,9,10,11,11a-octahydro-5H-dibenzo[a,c]cycloheptene-3-carboxylic acid (0.125 g, 0.378 mmol), HBTU (0.172 g, 0.454 mmol) and DIEA (0.10 mL, 0.567 mmol) in DMF (3 mL) was stirred at about 0° C. for about 10 min Benzene-1,2-diamine (0.123 g, 1.135 mmol) was added and mixture was warmed to rt and stirred for about 18 h. Water (10 mL) was added and resulting solids were collected by filtration, rinsing with water. The residue was dried under reduced pressure at 60° C. to yield (7aS,9R,11aR)-11a-ethyl-9-hydroxy-9-propyl-6,7,7a,8,9,10,11,11a-octahydro-5H-dibenzo[a,c]cycloheptene-3-carboxylic acid (2-amino-phenyl)-amide; compound with (7aR,9S,11aS)-11a-ethyl-9-hydroxy-9-propyl-6,7,7a,8,9,10,11,11a-octahydro-5H-dibenzo[a,c]cycloheptene-3-carboxylic acid (2-amino-phenyl)-amide (85, R⁴=Methyl, R⁵=Ethyl, R⁶=2-Amino-phenyl) (0.148 g, 93%); LC/MS method 2, Rₜ=2.47 min, MS m/z 421 (M+H)⁺; ¹H NMR (400 MHz, DMSO-d₆) δ 9.54 (s, 1H), 7.72-7.67 (m, 2H), 7.31 (d, J=8.0 Hz, 1H), 7.12 (d, J=6.6 Hz, 1H), 6.97-6.90 (m, 1H), 6.75 (dd, J=8.0, 1.3 Hz, 1H), 6.60-6.54 (m, 1H), 4.85 (s, 2H), 3.88 (s, 1H), 3.01-2.91 (m, 1H), 2.92-2.82 (m, 1H), 2.35-2.15 (m, 3H), 2.08-1.99 (m, 1H), 1.75-1.60 (m, 2H), 1.57-1.35 (m, 5H), 1.27-0.98 (m, 6H), 0.75 (t, J=7.1 Hz, 3H), 0.60 (t, J=7.4 Hz, 3H).

### Example #146

### (3R,4aS,11bR)-9-(1H-Benzoimidazol-2-yl)-11b-ethyl-3-propyl-2,3,4,4a,5,6,7,11b-octahydro-1H-dibenzo[a,c]cyclohepten-3-ol; compound with (3S,4aR,11bS)-9-(1H-benzoimidazol-2-yl)-11b-ethyl-3-propyl-2,3,4,4a,5,6,7,11b-octahydro-1H-dibenzo[a,c]cyclohepten-3-ol

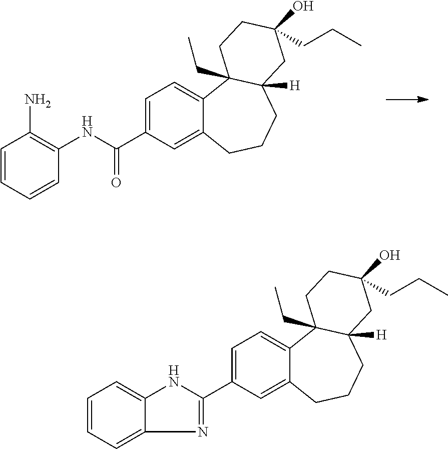

A solution of (7aS,9R,11aR)-11a-ethyl-9-hydroxy-9-propyl-6,7,7a,8,9,10,11,11a-octahydro-5H-dibenzo[a,c]cycloheptene-3-carboxylic acid (2-amino-phenyl)-amide; compound with (7aR,9S,11aS)-11a-ethyl-9-hydroxy-9-propyl-6,7,7a,8,9,10,11,11a-octahydro-5H-dibenzo[a,c]cycloheptene-3-carboxylic acid (2-amino-phenyl)-amide (85, R⁴=Methyl, R⁵=Ethyl, R⁶=2-Amino-phenyl) (0.133 g, 0.316 mmol) in acetic acid (1 mL) was heated to about 60° C. for about 3 h. The mixture was cooled to rt and concentrated under reduced pressure. The residue was purified on silica gel (12 g) using a gradient of 40-100% EtOAc in heptane to provide (3R,4aS,11bR)-9-(1H-benzoimidazol-2-yl)-11b-ethyl-3-propyl-2,3,4,4a,5,6,7,11b-octahydro-1 H-dibenzo[a,c]cyclohepten-3-ol; compound with (3S,4aR,11bS)-9-(1H-benzoimidazol-2-yl)-11b-ethyl-3-propyl-2,3,4,4a,5,6,7,11b-octahydro-1H dibenzo[a,c]cyclohepten-3-ol (0.082 g, 64%). LC/MS method 2, Rₜ=2.54 min, MS m/z 403 (M+H)⁺. ¹H NMR (400 MHz, DMSO-d₆) δ 12.75 (s, 1H), 7.92-7.83 (m, 2H), 7.62 (d, J=7.2 Hz, 1H), 7.49 (d, J=6.7 Hz, 1H), 7.35 (d, J=8.3 Hz, 1H), 7.21-7.11 (m, 2H), 3.88 (s, 1H), 3.05-2.95 (m, 1H), 2.93-2.80 (m, 1H), 2.33-2.15 (m, 3H), 2.11-1.99 (m, 1H), 1.72-1.60 (m, 2H), 1.57-1.36 (m, 5H), 1.26-1.05 (m, 6H), 0.74 (t, J=7.1 Hz, 3H), 0.62 (t, J=7.4 Hz, 3H).

### Example #147

### (3R,4aS,11bR)-9-(1H-Benzoimidazol-2-yl)-11b-ethyl-3-propyl-2,3,4,4a,5,6,7,11b-octahydro-1H-dibenzo[a,c]cyclohepten-3-ol and Example #148: (3S,4aR,11bS)-9-(1H-benzoimidazol-2-yl)-11b-ethyl-3-propyl-2,3,4,4a,5,6,7,11b-octahydro-1H-dibenzo[a,c]cyclohepten-3-ol

### Chiral Separation of Example #146

The enantiomers of Example #146 were separated using Preparative Chiral Purification Method 19. The first peak eluted was (3R,4aS,11bR)-9-(1H-benzoimidazol-2-yl)-11b-ethyl-3-propyl-2,3,4,4a, 5,6,7,11b-octahydro-1H-dibenzo[a,c]cyclohepten-3-ol (Example #147) and the second was (3S,4aR,11bS)-9-(1H-benzoimidazol-2-yl)-11b-ethyl-3-propyl-2,3,4,4a,5,6,7,11b-octahydro-1H-dibenzo[a,c]cyclohepten-3-ol (Example #148). NMR and LC/MS data for single isomers were essentially identical to the racemic mixture.

### Example #149

### (7aS,9R,11aR)-11a-Ethyl-9-hydroxy-9-propyl-6,7,7a,8,9,10,11,11a-octahydro-5H-dibenzo[a,c]cycloheptene-3-carboxylic acid (2-amino-pyridin-3-yl)-amide; compound with (7aR,9S,11aS)-11a-ethyl-9-hydroxy-9-propyl-6,7,7a,8,9,10,11,11a-octahydro-5H-dibenzo[a,c]cycloheptene-3-carboxylic acid(2-amino-pyridin-3-yl)-amide (85, R⁴=Methyl, R⁵=Ethyl, R⁶=2-Amino-pyridin-3-yl)

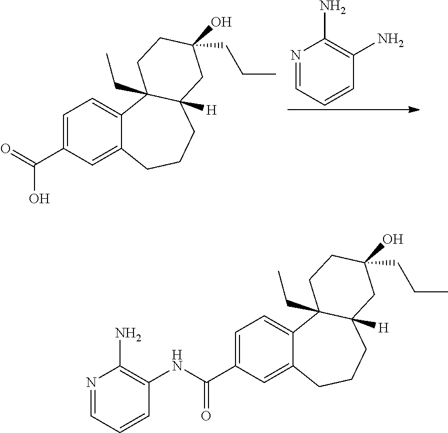

A solution of (7aS,9R,11aR)-11a-ethyl-9-hydroxy-9-propyl-6,7,7a,8,9,10,11,11a-octahydro-5H-dibenzo[a,c]cycloheptene-3-carboxylic acid; compound with (7aR,9S,11aS)-11a-ethyl-9-hydroxy-9-propyl-6,7,7a,8,9,10,11,11a-octahydro-5H-dibenzo[a,c]cycloheptene-3-carboxylic acid (0.049 g, 0.148 mmol), HBTU (0.067 g, 0.178 mmol), DIEA (0.039 mL, 0.222 mmol) and DMF (3 mL) was stirred at about 0° C. for about 10 min. Pyridine-2,3-diamine (0.049 g, 0.445 mmol) was added and the mixture was warmed to rt and stirred for about 18 h. Water (20 mL) was added and the resulting solids were collected by filtration, rinsing with water. The residue was dried under reduced pressure at about 60° C. to provide (7aS,9R,11aR)-11a-ethyl-9-hydroxy-9-propyl-6,7,7a,8,9,10,11,11a-octahydro-5H-dibenzo[a,c]cycloheptene-3-carboxylic acid (2-amino-pyridin-3-yl)-amide; compound with (7aR,9S,11aS)-11a-ethyl-9-hydroxy-9-propyl-6,7,7a,8,9,10,11,11a-octahydro-5H-dibenzo[a,c]cycloheptene-3-carboxylic acid(2-amino-pyridin-3-yl)-amide (85, R⁴=Methyl, R⁵=Ethyl, R⁶=2-Amino-pyridin-3-yl) (0.063 g, 100%). LC/MS method 2, Rₜ=2.11 min, MS m/z 422 (M+H)⁺. ¹H NMR (400 MHz, DMSO-d₆) δ 9.52 (s, 1H), 7.83 (dd, J=4.9, 1.7 Hz, 1H), 7.72-7.67 (m, 2H), 7.52-7.47 (m, 1H), 7.32 (d, J=8.1 Hz, 1H), 6.59 (dd, J=7.6, 4.9 Hz, 1H), 5.74 (s, 2H), 3.89 (s, 1H), 3.01-2.83 (m, 2H), 2.31-2.14 (m, 3H), 2.09-1.99 (m, 1H), 1.72-1.59 (m, 2H), 1.55-1.38 (m, 5H), 1.24-1.04 (m, 6H), 0.75 (t, J=7.1 Hz, 3H), 0.60 (t, J=7.4 Hz, 3H).

### Example #150

### ((7aS,9R,11aS)-11a-Cyclopropylmethyl-9-hydroxy-9-propyl-6,7,7a,8,9,10,11,11a-octahydro-5H-dibenzo[a,c]cycloheptene-3-carboxylic acid (2-methy 1-pyridin-3-yl)-amide; compound with (7aR,9S,11aS)-11a-cyclopropylmethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide (77, R⁴=Cyclopropyl, R⁵=Ethyl)

### Step 1: 5-(Cyclopropylmethyl)-2-methoxy-5,7,8,9-tetrahydro-benzocyclohepten-6-one (69, R⁴=Cyclopropyl)

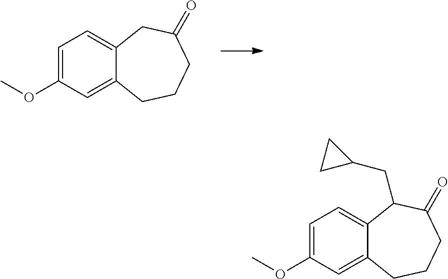

A solution of 2-methoxy-8,9-dihydro-5H-benzo[7]annulen-6(7H)-one (3) (80.0 g, 0.420 mol) in DMF (1.6 L) was cooled to about 0° C. and sodium hydride (11.1 g, 0.462 mol) was added. The mixture was stirred at about 0° C. for about 30 min then cyclopropylmethyl bromide (62.5 mL, 0.116 mol) was added. The resulting solution was stirred at rt for about 1 h. The compound was purified on silica gel to give 5-(cyclopropylmethyl)-2-methoxy-5,7,8,9-tetrahydro-benzocyclohepten-6-one (69, R⁴=Cyclopropyl) (27.0 g, 26%). LC/MS, method 3, Rₜ=2.48 min, MS m/z 245 (M+H)⁺. ¹H NMR (400 MHz, DMSO) δ 7.05 (d, J=8.1 Hz, 1H), 6.79-6.73 (m, 2H), 4.00-3.96 (m, 1H), 3.72 (s, 3H), 3.07-2.99 (m, 1H), 2.84-2.76 (m, 1H), 2.75-2.67 (m, 1H), 2.41-2.36 (m, 1H), 2.10-1.99 (m, 1H), 1.97-1.91 (m, 1H), 1.75-1.62 (m, 1H), 1.61-1.50 (m, 1H), 0.66-0.52 (m, 1H), 0.39-0.30 (m, 2H), 0.08-0.01 (m, 2H).

### Step #2: 11b-Cyclopropylmethyl-9-methoxy-1,2,5,6,7,11b-hexahydro-dibenzo[a,c]cyclohepten-3-one (70, R⁴=Cyclopropyl)

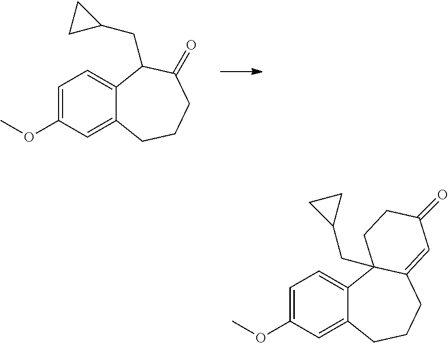

To EtOH (150 mL) under nitrogen was added freshly cut sodium (2.12 g, 92 0 mmol) portionwise and the mixture was stirred until the reaction was complete. A solution of 5-(cyclopropylmethyl)-2-methoxy-5,7,8,9-tetrahydro-benzocyclohepten-6-one (69, R⁴=Cyclopropyl) (15.0 g, 61.4 mmol) in EtOH (150 mL) was added. The mixture was stirred for about 10 min, then but-3-en-2-one (5.38 g, 77 mmol) was added over about 30 min. The mixture was stirred at rt for about 30 min. The mixture was treated with another portion of but-3-en-2-one (2.69 g, 38 4 mmol) then stirred for about 1 h. The mixture was then heated to about 60° C. for about 15 min then cooled to rt and stirred for about 12 h. The mixture was concentrated under reduced pressure then partitioned between EtOAc (200 mL) and water (100 mL). The aqueous layer was extracted with EtOAc (50 mL) then the combined organics were dried over MgSO₄, filtered and concentrated under reduced pressure. The residue was purified on silica gel (330 g) using a gradient of 0-40% EtOAc in heptane. Product fractions were combined and concentrated under reduced pressure to yield 11b-Cyclopropylmethyl-9-methoxy-1,2,5,6,7,11b-hexahydro-dibenzo[a,c]cyclohepten-3-one (70, R⁴=Cyclopropyl) (12.3 g, 67%). LC/MS, method 3, Rₜ=2.52 min, MS m/z 297 (M+H)⁺. ¹H NMR (400 MHz, DMSO-d₆) δ 7.37 (d, J=8.7 Hz, 1H), 6.84 (dd, J=8.7, 2.9 Hz, 1H), 6.72 (d, J=2.9 Hz, 1H), 5.83 (s, 1H), 3.73 (s, 3H), 2.91-2.73 (m, 2H), 2.63-2.51 (m, 2H), 2.49-2.37 (m, 2H), 2.33-2.17 (m, 2H), 1.89-1.80 (m, 2H), 1.79-1.72 (m, 1H), 1.47-1.32 (m, 1H), 0.70-0.55 (m, 1H), 0.46-0.27 (m, 2H), 0.15-0.01 (m, 2H).

### Step #3: 11b-Cyclopropylmethyl-9-hydroxy-1,2,5,6,7,11b-hexahydro-dibenzo[a,c]cyclohepten-3-one (71, R⁴=Cyclopropyl)

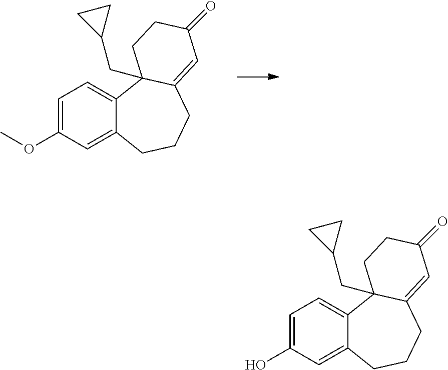

A mixture containing 11b-cyclopropylmethyl-9-methoxy-1,2,5,6,7,11b-hexahydro-dibenzo[a,c]cyclohepten-3-one (70, R⁴=Cyclopropyl) (12.3 g, 41.3 mmol) and DCM (225 mL) was cooled to about −10° C. then borontribromide (1M solution in DCM, 64 mL, 64 mmol) was added over about 10 min keeping the reaction temperature between about −5° C. and 0° C. After complete addition the mixture was stirred at about −7° C. for about 40 min. MeOH (50 mL) was added dropwise over about 30 min keeping the internal temperature at about 0° C. The mixture was stirred at about 0° C. for about 30 min then concentrated under reduced pressure. The material was dissolved in EtOAc (250 mL) then saturated aqueous sodium bicarbonate (250 mL) was added over about 15 min. The mixture was stirred for about 30 min then the layers were separated. The aqueous layer was extracted with EtOAc (100 mL) then the combined organics were dried over MgSO₄, filtered and concentrated under reduced pressure. The residue was purified on silica gel (330 g) using a gradient of 0-30% EtOAc in DCM. Product fractions were combined and concentrated under reduced pressure to yield 11b-cyclopropylmethyl-9-hydroxy-1,2,5,6,7,11b-hexahydro-dibenzo[a,c]cyclohepten-3-one (71, R⁴=Cyclopropyl) (8.69 g, 75%). LC/MS, method 3, Rₜ=2.05 min, MS m/z 283 (M+H)⁺. ¹H NMR (400 MHz, DMSO-d₆) 9.21 (s, 1H), 7.24 (d, J=8.6 Hz, 1H), 6.66 (dd, J=8.5, 2.7 Hz, 1H), 6.53 (d, J=2.7 Hz, 1H), 5.83 (s, 1H), 2.87-2.64 (m, 2H), 2.60-2.34 (m, 4H), 2.27-2.19 (m, 2H), 1.89-1.70 (m, 3H), 1.39-1.33 (m, 1H), 0.71-0.55 (m, 1H), 0.45-0.24 (m, 2H), 0.11-−0.10 (m, 2H).

### Step #4: (+/−) Compound 76 (R⁴=Cyclopropyl)

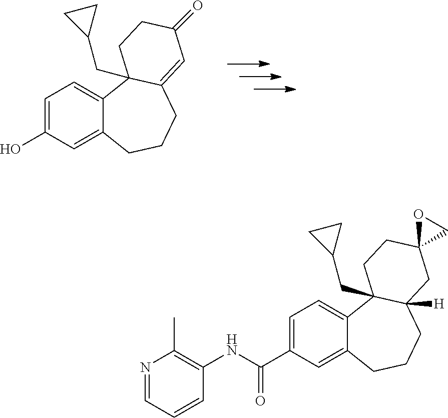

Compound 76 (R⁴=Cyclopropyl) was prepared in a manner similar to that described in Example #44 and #45, Steps 5 through 9 for the preparation of (+/−) Compound 76 (R⁴=Methyl) substituting 11b-cyclopropylmethyl-9-hydroxy-1,2,5,6,7,11b-hexahydro-dibenzo[a,c]cyclohepten-3-one (71, R⁴=Cyclopropyl) for 11b-ethyl-9-hydroxy-1,2,5,6,7,11b-hexahydro-dibenzo[a,c]cyclohepten-3-one (71, R⁴=Methyl) in step 5 to yield (+/−) Compound 76 (R⁴=Cyclopropyl). LC/MS, method 2, Rₜ=2.38 min, MS m/z 417 (M+H)⁺. ¹H NMR (400 MHz, DMSO-d₆) δ 9.94 (s, 1H), 8.31 (dd, J=4.7, 1.6 Hz, 1H), 7.79-7.69 (m, 3H), 7.49 (d, J=8.4 Hz, 1H), 7.25 (dd, J=7.9, 4.8 Hz, 1H), 3.25-3.19 (m, 1H), 3.05-2.98 (m, 1H), 2.91-2.86 (m, 1H), 2.78-2.74 (m, 1H), 2.55-2.51 (m, 2H), 2.42 (s, 3H), 2.28-2.11 (m, 3H), 2.05-1.96 (m, 1H), 1.83-1.64 (m, 3H), 1.59-1.55 (m, 1H), 1.48-1.43 (m, 1H), 1.28-1.14 (m, 1H), 0.79-0.76 (m, 1H), 0.47-0.30 (m, 2H), 0.21-0.10 (m, 1H), 0.06-0.01 (m, 1H), −0.28-−0.34 (m, 1H).

### Step #5: (7aR,9R,11aS)-11a-Cyclopropylmethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide; compound with (7aS,9S,11aR)-11a-cyclopropylmethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide (77, R⁴=Cyclopropyl, R⁵=Ethyl)

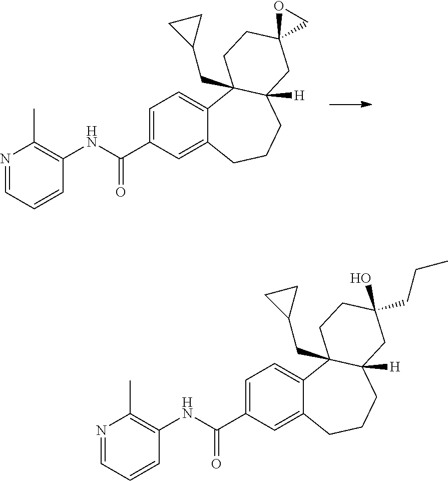

A round bottom flask with stirring bar, septum, nitrogen line and thermometer was charged with (+/−) Compound 76 (R⁴=Cyclopropyl) (0.33 g, 0.79 mmol)), THF (14 mL) and copper(I)iodide (0.025 g, 0.131 mmol). The mixture was cooled to an internal temperature of about 0° C. then ethylmagnesium bromide (3M solution in Et₂O, 1.6 mL, 4.8 mmol) was added dropwise maintaining internal temperature between 0° C. and 5° C. The mixture was stirred at about 0° C. for about 15 min then treated with saturated aqueous ammonium chloride (3 mL). The mixture was stirred for about 30 min then diluted with water (25 mL) and EtOAc (25 mL). The layers were separated and the aqueous layer extracted with EtOAc (15 mL). The combined organic solutions were dried over MgSO₄, filtered and concentrated under reduced pressure. The residue was purified on silica gel (12 g) using a gradient of 50-100% EtOAc in heptane. Product fractions were combined and concentrated under reduced pressure. The material was dissolved in MeOH (3 mL) then water (25 mL) was added. Partial concentration of the mixture under reduced pressure resulted in the formation of a solid which was collected by filtration and washed with water (5 mL). The material was dried under reduced pressure at about 60° C. to yield (7aR,9R,11aS)-11a-cyclopropylmethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide; compound with (7aS,9S,11aR)-11a-cyclopropylmethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide (77, R⁴=Cyclopropyl, R⁵=Ethyl) as a solid (0.275 g, 78%). LC/MS, method 2, Rₜ=2.47 min, MS m/z 447 (M+H)⁺. ¹H NMR (400 MHz, DMSO-d₆) δ 9.92 (s, 1H), 8.31 (dd, J=4.7, 1.5 Hz, 1H), 7.77-7.69 (m, 3H), 7.43 (d, J=8.4 Hz, 1H), 7.25 (dd, J=7.9, 4.8 Hz, 1H), 3.88 (s, 1H), 3.02-2.81 (m, 2H), 2.55-2.50 (m, 1H), 2.42 (s, 3H), 2.37-2.29 (m, 1H), 2.22-2.15 (m, 1H), 1.99-1.94 (m, 1H), 1.87-1.79 (m, 1H), 1.72-1.64 (m, 1H), 1.57-1.29 (m, 5H), 1.28-1.15 (m, 2H), 1.15-1.04 (m, 4H), 0.76 (t, J=7.1 Hz, 3H), 0.37-0.34 (m, 2H), 0.17-0.06 (m, 1H), 0.02-−0.02 (m, 1H), −0.34-−0.39 (m, 1H).

### Example #151

### (7aS,9R,11aR)-11a-Ethyl-9-hydroxy-9-propyl-6,7,7a,8,9,10,11,11a-octahydro-5H-dibenzo[a,c]cycloheptene-3-carboxylic acid (2-chloro-phenyl)-amide (85, R⁴=Methyl, R⁵=Ethyl, R⁶=2-chloro-phenyl)

### Step #1: Chiral separation of (7aS,9R,11aR)-11a-ethyl-9-hydroxy-9-propyl-6,7,7a,8,9,10,11,11a-octahydro-5H-dibenzo[a,c]cycloheptene-3-carboxylic acid methyl ester: compound with (7aR,9S,11aS)-11a-ethyl-9-hydroxy-9-propyl-6,7,7a,8,9,10,11,11a-octahydro-5H-dibenzo[a,c]cycloheptene-3-carboxylic acid methyl ester (80, R⁴=Methyl, R⁵=Ethyl)

The enantiomers were separated using Preparative Chiral Purification Method 4. The first peak eluted was (7aS,9R,11aR)-11a-ethyl-9-hydroxy-9-propyl-6,7,7a,8,9,10,11,11a-octahydro-5H-dibenzo[a,c]cycloheptene-3-carboxylic acid methyl ester (80, R⁴=Methyl, R⁵=Ethyl) and the second was (7aR,9S,11aS)-11a-ethyl-9-hydroxy-9-propyl-6,7,7a,8,9,10,11,11a-octahydro-5H-dibenzo[a,c]cycloheptene-3-carboxylic acid methyl ester (80, R⁴=Methyl, R⁵=Ethyl). NMR and LC/MS data for single isomers were essentially identical to the racemic mixture.

### Step #2: (7aS,9R,11aR)-11a-Ethyl-9-hydroxy-9-propyl-6,7,7a,8,9,10,11,11a-octahydro-5H-dibenzo[a,c]cycloheptene-3-carboxylic acid

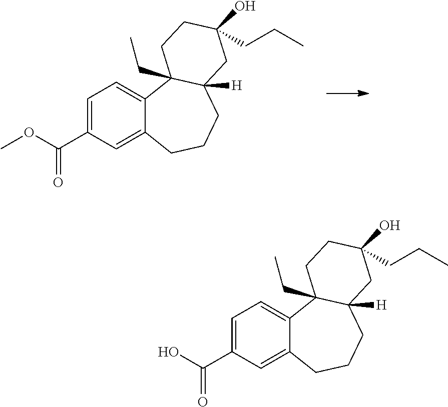

A solution of (7aS,9R,11aR)-11a-ethyl-9-hydroxy-9-propyl-6,7,7a,8,9,10,11,11a-octahydro-5H-dibenzo[a,c]cycloheptene-3-carboxylic acid methyl ester (80, R⁴=Methyl, R⁵=Ethyl) (0.212 g, 0.615 mmol) and LiOH (0.074 g, 3.1 mmol) in MeOH (3 mL) and water (3 mL) was heated at about 60° C. for about 16 h. The reaction temperature was increased to about 70° C. and additional LiOH (0.074 g, 3.1 mmol) was added. After about 3 h, the mixture was cooled to rt and 1M aqueous HCl was added drop-wise until a precipitate formed. The precipitate was collected by filtration, rinsing with water to yield (7aS,9R,11aR)-11a-ethyl-9-hydroxy-9-propyl-6,7,7a,8,9,10,11,11a-octahydro-5H-dibenzo[a,c]cycloheptene-3-carboxylic acid (0.121 g, 60%). LC/MS method 2, Rₜ=2.30 min, MS m/z 329 (M−H)⁻. ¹H NMR (400 MHz, DMSO-d₆) δ 7.57-7.50 (m, 2H), 7.12 (d, J=8.8 Hz, 1H), 3.89-3.75 (m, 1H), 2.94-2.83 (m, 1H), 2.79-2.70 (m, 1H), 2.29-2.08 (m, 3H), 2.05-1.93 (m, 1H), 1.66-1.64 (m, 2H), 1.53-1.30 (m, 5H), 1.23-0.98 (m, 6H), 0.74 (t, J=7.1 Hz, 3H), 0.57 (t, J=7.4 Hz, 3H).

### Step #3: (7aS,9R,11aR)-11a-Ethyl-9-hydroxy-9-propyl-6,7,7a,8,9,10,11,11a-octahydro-5H-dibenzo[a,c]cycloheptene-3-carboxylic acid (2-chloro-phenyl)-amide (85, R⁴=Methyl, R⁵=Ethyl, R⁶=2-chloro-phenyl)

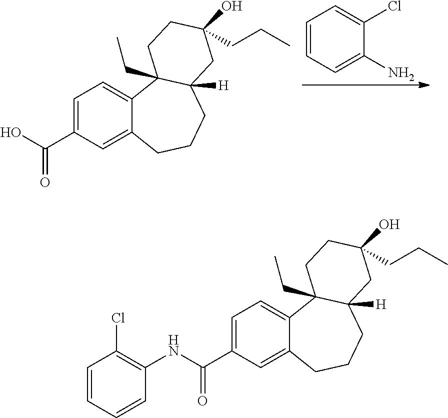

A solution of (7aS,9R,11aR)-11a-ethyl-9-hydroxy-9-propyl-6,7,7a,8,9,10,11,11a-octahydro-5H-dibenzo[a,c]cycloheptene-3-carboxylic acid (0.030 g, 0.091 mmol), HBTU (0.041 g, 0.109 mmol), DIEA (0.024 mL, 0.136 mmol) and DMF (1 mL) was stirred at rt for about 10 min. 2-Chloroaniline (0.035 g, 0.27 mmol) was added and the reaction was stirred for about 16 h at rt. The mixture was then heated to about 60° C. for about 24 h and then stirred at rt for about 24 h. The mixture was concentrated to dryness and then purified on silica gel (4 g) eluting with 10-50% EtOAc in heptane to yield (7aS,9R,11aR)-11a-ethyl-9-hydroxy-9-propyl-6,7,7a,8,9,10,11,11a-octahydro-5H-dibenzo[a,c]cycloheptene-3-carboxylic acid (2-chloro-phenyl)-amide (0.006 g, 15%). LC/MS method 2, Rₜ=3.24 min, MS m/z 440 & 442 (M+H)⁺. ¹H NMR (400 MHz, DMSO-d₆) δ 9.93 (s, 1H), 7.76-7.70 (m, 2H), 7.59-7.52 (m, 2H), 7.42-7.34 (m, 2H), 7.32-7.25 (m, 1H), 3.91 (s, 1H), 3.05-2.95 (m, 1H), 2.92-2.81 (m, 1H), 2.35-2.15 (m, 3H), 2.13-1.95 (m, 1H), 1.75-1.65 (m, 2H), 1.57-1.37 (m, 5H), 1.26-1.16 (m, 2H), 1.16-1.04 (m, 4H), 0.77 (t, J=7.1 Hz, 3H), 0.62 (t, J=7.4 Hz, 3H).

Fluorescense polarization binding ranges measured using GR Florescence Polarization Assay:

A=a compound with an IC₅₀ less than 0.1 μM
B=a compound with an IC₅₀ within the range of 0.1 to 1.0 μM
C=a compound with an IC₅₀ within the range of 1.0 to 10.0 μM
D=a compound with an IC₅₀ greater than 10 μM.

| Example | GR binding |
|---|---|
| 1 | B |
| 2 | B |
| 3 | A |
| 4 | B |
| 5 | B |
| 6 | A |
| 7 | A |
| 8 | A |
| 9 | A |
| 10 | A |
| 11 | A |
| 12 | A |
| 13 | B |
| 14 | A |
| 15 | B |
| 16 | B |
| 17 | A |
| 18 | A |
| 19 | A |
| 20 | B |
| 21 | D |
| 22 | D |
| 23 | A |
| 24 | A |
| 25 | C |
| 26 | A |
| 27 | C |
| 28 | B |
| 29 | B |
| 30 | A |
| 31 | A |
| 32 | A |
| 33 | A |
| 34 | A |
| 35 | C |
| 36 | D |
| 37 | D |
| 38 | C |
| 39 | D |
| 40 | D |
| 41 | D |
| 42 | B |
| 43 | B |
| 44 | B |
| 45 | A |
| 46 | B |
| 47 | A |
| 48 | B |
| 49 | A |
| 50 | B |
| 51 | A |
| 52 | A |
| 53 | A |
| 54 | A |
| 55 | A |
| 56 | B |
| 57 | A |
| 58 | A |
| 58A | A |
| 58B | A |
| 59 | C |
| 60 | A |
| 61 | B |
| 62 | A |
| 63 | A |
| 64 | B |
| 65 | A |
| 66 | A |
| 67 | B |
| 68 | A |
| 69 | A |
| 70 | A |
| 71 | A |
| 72 | A |
| 73 | A |
| 74 | B |
| 75 | A |
| 76 | A |
| 77 | B |
| 78 | A |
| 79 | A |
| 80 | A |
| 81 | A |
| 82 | B |
| 83 | A |
| 84 | D |
| 85 | A |
| 86 | A |
| 87 | A |
| 88 | A |
| 89 | B |
| 90 | A |
| 91 | B |
| 92 | A |
| 93 | A |
| 94 | B |
| 95 | A |
| 96 | A |
| 97 | A |
| 98 | A |
| 99 | C |
| 100 | A |
| 101 | A |
| 102 | A |
| 103 | A |
| 104 | B |
| 105 | A |
| 105A | A |
| 106 | A |
| 107 | B |
| 108 | C |
| 109 | D |
| 110 | D |
| 111 | A |
| 112 | B |
| 113 | C |
| 114 | A |
| 115 | B |
| 116 | B |
| 117 | B |
| 118 | A |
| 119 | A |
| 120 | B |
| 121 | A |
| 122 | A |
| 123 | B |
| 124 | C |
| 125 | A |
| 126 | A |
| 127 | B |
| 128 | A |
| 129 | B |
| 130 | A |
| 131 | A |
| 132 | B |
| 133 | A |
| 134 | A |
| 135 | B |
| 136 | A |
| 137 | C |
| 138 | A |
| 139 | A |
| 140 | B |
| 141 | D |
| 142 | A |
| 143 | A |
| 144 | C |
| 145 | A |
| 146 | B |
| 147 | C |
| 148 | C |
| 149 | A |
| 150 | A |
| 151 | A |

What is claimed:

1. A compound of Formula (I) 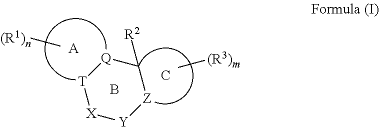
pharmaceutically acceptable salts, pro-drugs, biologically active metabolites, isomers, and stereoisomers wherein
Ring A is optionally substituted aryl, optionally substituted saturated or partially unsaturated (C₅-C₆)carbocyclyl or optionally substituted heteroaryl;
Ring C is optionally substituted saturated or partially unsaturated (C₅-C₆)carbocyclyl or optionally substituted heterocyclyl;
Q and T are independently C or N, provided that both are not N;
Ring B is a seven membered ring wherein
X is —C(R⁵)₂—, —C(R⁵)—, —C(═O)—, —N(R^{a})—, —O—, —S—, —S(O)—, or —S(O)₂—; or
when X is —C(R⁵)₂—, it can form a cyclopropyl ring spiro to the carbon atom to which it is attached;
Y is —C(R⁵)₂C(R⁵)₂—, —C(R⁵)C(R⁵)₂—, —C(R⁵)₂C(R⁵)—, —OC(R⁵)₂—, —N(R^{a})C(R⁵)₂—, —C(R⁵)₂N(R^{a})—, —C(═O)C(R⁵)₂—, —C(R⁵)₂C(═O)—, —O—C(═O)—, —C(═O)—O—, or —C(R⁵)₂—O—; or
Y is —C(R⁵)₂— when Q or T is N;
Z is CR⁴ or N; or
Ring B is a six membered ring wherein
Y is —C(R⁵)₂—;
Q or T must be N;
Z is CR⁴ or N; or
when X is —C(R⁵)₂—, it can form a cyclopropyl ring spiro to the carbon atom to which it is attached;
provided that X—Y or Y—Z do not form O—O, N—N, N—O, C(═O)—C(═O), N—C—O or O—C—O bonds; and
provided that in X—Y a sulfur atom is not adjacent to an oxygen atom or —C(═O);
provided that X—Y does not form —O—C(R⁵)₂—O—, —N—C(R⁵)₂—O— or —S—C(R⁵)₂—O—;
R¹ is H, Br, Cl, F, —COOR^{a}, —OR^{a}, —O-optionally substituted (C₁-C₃)alkylene-optionally substituted aryl, —O-optionally substituted (C₁-C₃)alkylene-optionally substituted heteroaryl, —O-optionally substituted (C₁-C₃)alkylene-optionally substituted heterocyclyl, optionally substituted (C₁-C₃)alkyl, optionally substituted aryl, optionally substituted (C₃-C₆)cycloalkyl, optionally substituted heteroaryl, optionally substituted heterocyclyl, —C(O)N(R^{a})(CH₂)ᵣ—R^{b}, —N(R^{a})C(O)(CH₂)ᵣ—R^{b}, —S(O)₂N(R^{a})—R^{b}—N(R^{a})S(O)₂—R^{b}, —O—S(O)₂—CF₃, —N(R^{a})-optionally substituted (C₃-C₆)cycloalkyl, —N(R^{a})-optionally substituted heterocyclyl, —N(R^{a})-optionally substituted heteroaryl, —N(R^{a})-optionally substituted aryl, 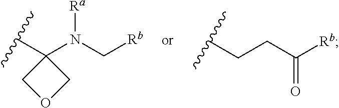
R² is —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl;
R³ is independently H, deuterium, —CD₃, —CF₃, optionally substituted (C₂-C₆)alkynyl, oxo, —OR^{a}, —OP(═O)(OH)(OH), optionally substituted (C₁-C₄)alkyl, —(C(R^{a})₂)ᵣ-optionally substituted (C₃-C₆)cycloalkyl, —(C(R^{a})₂)ᵣ-optionally substituted aryl, —(C(R^{a})₂)ᵣ-optionally substituted heteroaryl, —(C(R^{a})₂)ᵣ—N(R^{a})-optionally substituted heteroaryl, a carbocyclic or heterocyclic spirocyclic moiety attached to ring C;
R⁴ is H, optionally substituted (C₁-C₃)alkyl, OH or —O-optionally substituted (C₁-C₃)alkyl;
R⁵ is independently H, F, N(R^{a}), OR^{a}, optionally substituted (C₃-C₆)cycloalkyl, or optionally substituted (C₁-C₃)alkyl;
R^{a} is independently H, optionally substituted (C₃-C₆)cycloalkyl or optionally substituted (C₁-C₃)alkyl;
R^{b} is H, optionally substituted (C₁-C₃)alkyl, optionally substituted aryl, optionally substituted (C₃-C₆)cycloalkyl, optionally substituted heteroaryl or optionally substituted heterocyclyl;
m is 1, 2, 3 or 4;
n is 1, 2, 3 or 4; and
r is independently 0, 1 or 2.

2. The compound of claim 1 wherein the compound is of Formula (I)a or Formula (I)b

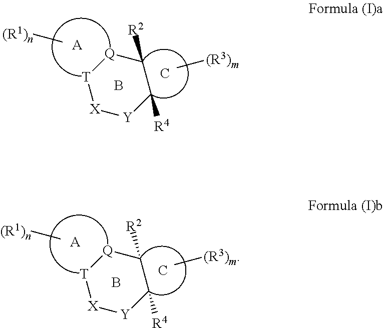

3. The compound of claim 2 wherein Ring A is optionally substituted phenyl, optionally substituted pyrrolyl, or optionally substituted pyrazolyl.

4. The compound of claim 3 wherein Ring C is optionally substituted cyclohexyl or optionally substituted cyclohexenyl.

5. The compound of claim 4 wherein X is —C(R⁵)₂—, —C(R⁵)—, —C(═O)—, —O— or —N(R^{a})—.

6. The compound of claim 5 wherein R¹ is —COOR^{a}, OR^{a}, optionally substituted (C₁-C₃)alkyl, —C(O)N(R^{a})(CH₂)ᵣ—R^{b}, —N(R^{a})C(O)(CH₂)ᵣ—R^{b}, optionally substituted azabenzimidazolyl, optionally substituted benzimidazolyl, —O-optionally substituted (C₁-C₃)alkylene-optionally substituted phenyl, or —O-optionally substituted (C₁-C₃)alkylene-optionally substituted pyridinyl.

7. The compound of claim 6 wherein R² is —CH₂CF₃, —(CH₂)ᵣ-optionally substituted aryl, or optionally substituted (C₁-C₃)alkyl.

8. The compound of claim 7 wherein R³ is independently H, —CF₃, —C≡CCH₃, oxo, —OR^{a}, —OP(═O)(OH)(OH), optionally substituted (C₁-C₄)alkyl, —(C(R^{a})₂)ᵣ-optionally substituted (C₃-C₆)cycloalkyl, or —(CH₂)ᵣ-optionally substituted aryl.

9. The compound of claim 8 wherein R³ is independently H, —CF₃, —C≡CCH₃, oxo, —OR^{a}, optionally substituted (C₁-C₄)alkyl, —CH₂-optionally substituted cyclopropyl, —CH₂-optionally substituted phenyl, or -optionally substituted phenyl.

10. The compound of claim 9 wherein R^{b} is H, optionally substituted azetidinyl, optionally substituted phenyl, optionally substituted piperidinyl, optionally substituted pyrimidinyl, optionally substituted pyridinyl, optionally substituted pyrazolyl, optionally substituted pyrrolidinyl or optionally substituted tetrazolyl.

11. The compound of claim 10 wherein Q is C.

12. The compound of claim 11 wherein T is C.

13. A compound of Formula (I) wherein the compound is
(4aR,11bS)-11b-benzyl-9-hydroxy-1,2,4,4a,5,6,7,11b-octahydro-dibenzo[a,c]cyclohepten-3-one; compound with (4aS,11bR)-11b-benzyl-9-hydroxy-1,2,4,4a,5,6,7,11b-octahydro-dibenzo[a,c]cyclohepten-3-one;
(3R,4aS,11bS)-11b-Benzyl-3-methyl-2,3,4,4a,5,6,7,11b-octahydro-1H-dibenzo[a,c]cycloheptene-3,9-diol; compound with (3S,4aR,11bR)-11b-benzyl-3-methyl-2,3,4,4a,5,6,7,11b-octahydro-1H-dibenzo[a,c]cycloheptene-3,9-diol;
(3R,4aR,11bR)-11b-benzyl-3-methyl-2,3,4,4a,5,6,7,11b-octahydro-1H-dibenzo[a,c]cycloheptene-3,9-diol; compound with (3S,4aS,11bS)-11b-benzyl-3-methyl-2,3,4,4a,5,6,7,11b-octahydro-1H-dibenzo[a,c]cycloheptene-3,9-diol;
(7aS,11aS)-11a-Benzyl-9-oxo-6,7,7a,8,9,10,11,11a-octahydro-5H-dibenzo[a,c]cycloheptene-3-carboxylic acid (2-methyl-pyridin-3-yl)-amide; compound with (7aR,11aR)-11a-benzyl-9-oxo-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9S,11aS)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide;
(7aR,9S,11aR)-11a-benzyl-9-ethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9S,11aS)-11a-Benzyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-11a-benzyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-methoxymethyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-hydroxy-9-methoxymethyl-6,7,7a,8,9,10,11,11a-octahydro-5H-dibenzo[a,c]cycloheptene-3-carboxylic acid (2-methyl-pyridin-3-yl)-amide;
(7aR,11aS)-11a-Benzyl-9-hydroxy-N-(2-methylpyridin-3-yl)-6-oxo-9-(trifluoromethyl)-6,7,7a,8,9,10,11,11a-octahydro-5H-dibenzo[b,d]azepine-3-carboxamide;
(4aS,11bS)-11b-benzyl-3-hydroxy-N-(2-methylpyridin-3-yl)-7-oxo-3-(trifluoromethyl)-2,3,4,4 a,5,6,7,11b-octahydro-1H-dibenzo[c,e]azepine-9-carboxamide;
(7aS,9R,11aS)-11a-Benzyl-9-ethyl-9-hydroxy-5-oxo-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-ethyl-9-hydroxy-5-oxo-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-ethyl-9-hydroxy-5-oxo-6,7,7a,8,9,10,11,11a-octahydro-5H-dibenzo[a,c]cycloheptene-3-carboxylic acid (2-methyl-pyridin-3-yl)-amide;
(7aR,9S,11aR)-11a-benzyl-9-ethyl-9-hydroxy-5-oxo-6,7,7a,8,9,10,11,11a-octahydro-5H-dibenzo[a,c]cycloheptene-3-carboxylic acid (2-methyl-pyridin-3-yl)-amide;
(7aR,9S,11aS)-9-Ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide; compound with (7aS,9R,11aR)-9-ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide;
(7aR,9R,11aS)-9-ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide; compound with (7aS,9S,11aR)-9-ethyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide;
(3S,4aS,11bS)-11b-Benzyl-3-prop-1-ynyl-2,3,4,4 a,5,6,7,11b-octahydro-1H-dibenzo[a,c]cycloheptene-3,9-diol; compound with (3R,4 aR,11bR)-11b-benzyl-3-prop-1-ynyl-2,3,4,4 a,5,6,7,11b-octahydro-1H-dibenzo[a,c]cycloheptene-3,9-diol;
(7aS,9S,11aS)-11a-Benzyl-9-hydroxy-9-prop-1-ynyl-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-11a-benzyl-9-hydroxy-9-prop-1-ynyl-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-propyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-hydroxy-9-propyl-6,7,7a,8,9,10,11,11a-octahydro-5H-dibenzo[a,c]cycloheptene-3-carboxylic acid (2-methyl-pyridin-3-yl)-amide;
(7aS,9S,11aS)-11a-Benzyl-9-ethynyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aS,9S,11aS)-11a-benzyl-9-ethynyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-ethoxymethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-benzyl-9-ethoxymethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aR)-9-Benzyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide; compound with (7aR,9S,11aS)-9-benzyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide;
(7aS,9S,11aR)-9-benzyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide; compound with (7aR,9R,11aS)-9-benzyl-9-hydroxy-11a-propyl-6,7,7a,8,9,10,11,11a-octahydro-5H-dibenzo[a,c]cycloheptene-3-carboxylic acid (2-methyl-pyridin-3-yl)-amide;
(6aS,8R,10aS)-10a-Benzyl-8-ethyl-1-methyl-1,4,5,6,6a,7,8,9,10,10a-decahydro-1,2-diaza-benzo[e]azulen-8-ol; compound with (6aR,8S,10aR)-10a-benzyl-8-ethyl-1-methyl-1,4,5,6,6a,7,8,9,10,10a-decahydro-1,2-diaza-benzo[e]azulen-8-ol;
(6aS,8S,10aS)-10a-Benzyl-8-ethyl-1-methyl-1,4,5,6,6a,7,8,9,10,10a-decahydro-1,2-diaza-benzo[e]azulen-8-ol; compound with (6aR,8R,10aR)-10a-benzyl-8-ethyl-1-methyl-1,4,5,6,6a,7,8,9,10,10a-decahydro-1,2-diaza-benzo[e]azulen-8-ol;
(6aS,8R,10 aS)-10a-Benzyl-8-ethyl-2-methyl-2,4,5,6,6a,7,8,9,10,10a-decahydro-1,2-diaza-benzo[e]azulen-8-ol; compound with (6aR,8S,10aR)-10a-benzyl-8-ethyl-2-methyl-2,4,5,6,6a,7,8,9,10,10a-decahydro-1,2-diaza-benzo[e]azulen-8-ol;
(6aS,8S,10 aS)-10a-benzyl-8-ethyl-2-methyl-2,4,5,6,6a,7,8,9,10,10a-decahydro-1,2-diaza-benzo[e]azulen-8-ol; compound with (6aR,8R,10aR)-10a-benzyl-8-ethyl-2-methyl-2,4,5,6,6a,7,8,9,10,10a-decahydro-1,2-diaza-benzo[e]azulen-8-ol;
(2R,3R,4aS,11bR)-11b-Benzyl-3-phenyl-2,3,4,4a,5,6,7,11b-octahydro-1H-dibenzo[a,c]cycloheptene-2,3,9-triol compound with (2S,3S,4aR,11bS)-11b-benzyl-3-phenyl-2,3,4,4a,5,6,7,11b-octahydro-1H-dibenzo[a,c]cycloheptene-2,3,9-triol;
(7aS,9R,10R,11aR)-11a-Benzyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide; compound with (7aR,9S,10S,11aS)-11a-benzyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide;
(7aR,9S,11aS)-11a-Ethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide;
(7aS,9R,11aR)-11a-Ethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide;
(7aR,9S,11aS)-9,11a-Diethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aR)-9,11a-Diethyl-9-hydroxy-6,7,7a,8,9,10,11,11a-octahydro-5H-dibenzo[a,c]cycloheptene-3-carboxylic acid (2-methyl-pyridin-3-yl)-amide;
(7aR,9R,11aS)-11a-Ethyl-9-hydroxy-9-isobutyl-6,7,7a,8,9,10,11,11a-octahydro-5H-dibenzo[a,c]cycloheptene-3-carboxylic acid (2-methyl-pyridin-3-yl)-amide;
(7aS,9S,11aR)-11a-Ethyl-9-hydroxy-9-isobutyl-6,7,7a,8,9,10,11,11a-octahydro-5H-dibenzo[a,c]cycloheptene-3-carboxylic acid (2-methyl-pyridin-3-yl)-amide;
(7aR,9R,11aS)-9-Cyclopropylmethyl-11a-ethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9S,11aR)-9-Cyclopropylmethyl-11a-ethyl-9-hydroxy-6,7,7a, 8,9,10,11,11a-octahydro-5H-dibenzo[a,e]cycloheptene-3-carboxylic acid (2-methyl-pyridin-3-yl)-amide;
(7aS,9R,11aS)-9-Hydroxy-9-propyl-11a-(2,2,2-trifluoro-ethyl)-6,7,7a, 8,9,10,11,11a-octahydro-5H-dibenzo[a,c]cycloheptene-3-carboxylic acid (2-methyl-pyridin-3-yl)-amide; compound with (7aR,9S,11aR)-9-hydroxy-9-propyl-11a-(2,2,2-trifluoro-ethyl)-6,7,7a, 8,9,10,11,11a-octahydro-5H-dibenzo[a,c]cycloheptene-3-carboxylic acid (2-methyl-pyridin-3-yl)-amide;
(7aS,9S,11aR)-11a-Ethyl-9-hydroxy-9-isobutyl-6,7,7a,8,9,10,11,11a-octahydro-5-oxa-dibenzo[a,c]cycloheptene-3-carboxylic acid (2-methyl-pyridin-3-yl)-amide; compound with (7aR,9R,11aS)-11a-Ethyl-9-hydroxy-9-isobutyl-6,7,7a,8,9,10,11,11a-octahydro-5-oxa-dibenzo[a,c]cycloheptene-3-carboxylic acid (2-methyl-pyridin-3-yl)-amide;
(7aS,9S,11aR)-9-Cyclopropylmethyl-11a-ethyl-9-hydroxy-6,7,7a,8,9,10,11,11a-octahydro-5-oxa-dibenzo[a,c]cycloheptene-3-carboxylic acid (2-methyl-pyridin-3-yl)-amide; compound with (7aR,9R,11aS)-9-Cyclopropylmethyl-11a-ethyl-9-hydroxy-6,7,7a,8,9,10,11,11a-octahydro-5-oxa-dibenzo[a,c]cycloheptene-3-carboxylic acid (2-methyl-pyridin-3-yl)-amide;
(7aS,9S,11aR)-11a-Ethyl-9-hydroxy-9-isobutyl-6,7,7a,8,9,10,11,11a-octahydro-5-oxa-dibenzo[a,c]cycloheptene-3-carboxylic acid (2-methyl-pyridin-3-yl)-amide;
(7aR,9R,11aS)-11a-Ethyl-9-hydroxy-9-isobutyl-6,7,7a,8,9,10,11,11a-octahydro-5-oxa-dibenzo[a,c]cycloheptene-3-carboxylic acid (2-methyl-pyridin-3-yl)-amide;
(7aS,9S,11aR)-9-Cyclopropylmethyl-11a-ethyl-9-hydroxy-6,7,7a,8,9,10,11,11a-octahydro-5-oxa-dibenzo[a,c]cycloheptene-3-carboxylic acid (2-methyl-pyridin-3-yl)-amide;
(7aS,9R,11aS)-9-Cyclopropylmethyl-11a-ethyl-9-hydroxy-6,7,7a,8,9,10,11,11a-octahydro-5-oxa-dibenzo[a,c]cycloheptene-3-carboxylic acid (2-methyl-pyridin-3-yl)-amide;
(7aS,9R,11aS)-9-Hydroxy-9-propyl-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide;
(7aR,9S,11aR)-9-Hydroxy-9-propyl-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide;
(7aR,9S,11aS)-11a-Ethyl-9-hydroxy-9-methyl-6,7,7a,8,9,10,11,11a-octahydro-5H-dibenzo[a,c]cycloheptene-3-carboxylic acid (2-methyl-pyridin-3-yl)-amide;
(7aS,9R,11aR)-11a-ethyl-9-hydroxy-9-methyl-6,7,7a,8,9,10,11,11a-octahydro-5H-dibenzo[a,c]cycloheptene-3-carboxylic acid (2-methyl-pyridin-3-yl)-amide;
(7aR,9R,11aS)-11a-Ethyl-9-hydroxy-5-oxo-9-propyl-6,7,7a,8,9,10,11,11a-octahydro-5H-dibenzo[a,c]cycloheptene-3-carboxylic acid (2-methyl-pyridin-3-yl)-amide;
(7aS,9S,11aR)-11a-Ethyl-9-hydroxy-5-oxo-9-propyl-6,7,7a,8,9,10,11,11a-octahydro-5H-dibenzo[a,c]cycloheptene-3-carboxylic acid (2-methyl-pyridin-3-yl)-amide;
(7aS,9S,11aS)-9-Hydroxy-9-isobutyl-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide; compound with (7aR,9R,11aR)-9-hydroxy-9-isobutyl-11a-(2,2,2-trifluoro-ethyl)-6,7,7a,8,9,10,11,11a-octahydro-5H-dibenzo[a,c]cycloheptene-3-carboxylic acid (2-methyl-pyridin-3-yl)-amide;
(7aS,9R,11aR)-9-Cyanomethyl-11a-ethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aS)-9-cyanomethyl-11a-ethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-cyanomethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-Benzyl-9-cyanomethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-cyanomethyl-9-hydroxy-6,7,7a,8,9,10,11,11a-octahydro-5H-dibenzo[a,c]cycloheptene-3-carboxylic acid (2-methyl-pyridin-3-yl)-amide;
(7aR,9S,11aR)-11a-Benzyl-9-cyanomethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2,4-dimethyl-pyrimidin-5-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (3,5-dimethyl-pyrazin-2-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (3-methyl-pyridin-4-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2,6-dimethyl-pyridin-3-yl)-amide;
(7aS,9R,11aS)-11a-Benzyl-9-ethyl-9-hydroxy-6,7,7a,8,9,10,11,11a-octahydro-5H-dibenzo[a,c a,c]cycloheptene-3-carboxylic acid (3-methyl-pyridin-2-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid [1,3,4]thiadiazol-2-ylamide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2-methyl-2H-pyrazol-3-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2,5-dimethyl-2H-pyrazol-3-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2,4-dimethyl-pyrimidin-5-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (1-methyl-1H-tetrazol-5-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (5-methyl-2H-pyrazol-3-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2-methyl-pyridin-3-ylmethyl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2-morpholin-4-yl-ethyl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (1-methyl-4-oxo-4,5-dihydro-1H-imidazol-2-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2-ethyl-2H-pyrazol-3-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid [2-methyl-6-(2H-pyrazol-3-yl)-pyridin-3-yl]-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid [2-methyl-6-(1H-pyrazol-4-yl)-pyridin-3-yl]-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid methyl-(2-methyl-pyridin-3-yl)-amide;
(7aS,9R,11aR)-11a-Ethyl-9-hydroxy-9-(2,2,2-trifluoro-ethoxymethyl)-6,7,7a,8,9,10,11,11a-octahydro-5H-dibenzo[a,c]cycloheptene-3-carboxylic acid (2-methyl-pyridin-3-yl)-amide: compound with (7aR,9S,11aS)-11a-ethyl-9-hydroxy-9-(2,2,2-trifluoro-ethoxymethyl)-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-ethoxymethyl-9-hydroxy-6,7,7a,8,9,10,11,11a-octahydro-5H-dibenzo[a,c]cycloheptene-3-carboxylic acid (2-methyl-pyridin-3-yl)-amide;
(7aR,9S,11aR)-11a-Benzyl-9-ethoxymethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-(2,2,2-trifluoro-ethoxymethyl)-6,7,7a,8,9,10,11,11a-octahydro-5H-dibenzo[a,c]cycloheptene-3-carboxylic acid (2-methyl-pyridin-3-yl)-amide; compound with (7aR,9S,11aR-11a-Benzyl-9-hydroxy-9-(2,2,2-trifluoro-ethoxymethyl)-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-(oxetan-3-ylmethoxymethyl)-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-Benzyl-9-hydroxy-9-(oxetan-3-ylmethoxymethyl)-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-isopropoxymethyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-Benzyl-9-hydroxy-9-isopropoxymethyl-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-prop oxymethyl-6,7,7a,8,9,10,11,11a-octahydro-5H-dibenzo[a,c]cycloheptene-3-carboxylic acid (2-methyl-pyridin-3-yl)-amide;
(7aS,9S,11aR-11a-Benzyl-9-hydroxy-9-prop oxymethyl-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-(2,2,2-trifluoro-1-methyl-ethoxymethyl)-5,7,7a,8,9,10,11,11a-octahydro-dibenzo[c,e]oxepine-3-carboxylic acid (2-methyl-pyridin-3-yl)-amide; compound with (7aR,9S,11aR)-11a-Benzyl-9-hydroxy-9-(2,2,2-trifluoro-1-methyl-ethoxymethyl)-5,7,7a,8,9,10,11,11a-octahydro-dibenzo[c,e]oxepine-3-carboxylic acid (2-methyl-pyridin-3-yl)-amide;
(7aS,9R,11aS)-11a-Benzyl-9-hydroxy-9-prop oxymethyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR-11a-Benzyl-9-hydroxy-9-propoxymethyl-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-(tetrahydro-pyran-4-yloxymethyl)-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-Benzyl-9-hydroxy-9-(tetrahydro-pyran-4-yloxymethyl)-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-phenoxymethyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-Benzyl-9-hydroxy-9-phenoxymethyl-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-hydroxymethyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-Benzyl-9-hydroxy-9-hydroxymethyl-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-9-(2-methanesulfonyl-ethoxymethyl)-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-Benzyl-9-hydroxy-9-(2-methanesulfonyl-ethoxymethyl)-6,7,7a,8,9,10,11,11a-octahydro-5H-dibenzo[a,c]cycloheptene-3-carboxylic acid (2-methyl-pyridin-3-yl)-amide;
(7aS,9R,11aR)-9-Ethoxymethyl-11a-ethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aS)-9-ethoxymethyl-11a-ethyl-9-hydroxy-6,7,7a,8,9,10,11,11a-octahydro-5H-dibenzo[a,c]cycloheptene-3-carboxylic acid (2-methyl-pyridin-3-yl)-amide;
(7aS,9R,11aR)-9-Ethoxymethyl-11a-ethyl-9-hydroxy-6,7,7a,8,9,10,11,11a-octahydro-5H-dibenzo[a,c]cycloheptene-3-carboxylic acid (2-methyl-pyridin-3-yl)-amide; compound with (7aR,9S,11aS)-9-ethoxymethyl-11a-ethyl-9-hydroxy-6,7,7a,8,9,10,11,11a-octahydro-5H-dibenzo[a,c]cycloheptene-3-carboxylic acid (2-methyl-pyridin-3-yl)-amide;
(4aS,11bS)-11b-Benzyl-6-methyl-N-(2-methylpyridin-3-yl)-3-oxo-2,3,4,4a,5,6,7,11b-octahydro-1H-dibenzo[c,e]azepine-9-carb oxamide;
(3S,4aS,11bS)-11b-benzyl-3-hydroxy-6-methyl-N-(2-methylpyridin-3-yl)-2,3,4,4a,5,6,7,11b-octahydro-1H-dibenzo[c,e]azepine-9-carboxamide;
(7aS,11aS)-11a-Benzyl-N-(2-methylpyridin-3-yl)-7,9-dioxo-5,7,7a,8,9,10,11,11a-octahydrodibenzo[c,e]oxepine-3-carboxamide;
(7aS,9R,11aS)-11a-Benzyl-9-ethyl-9-hydroxy-N-(2-methylpyridin-3-yl)-5,7,7a,8,9,10,11,11a-octahydrodibenzo[c,e]oxepine-3-carboxamide;
(7aS,9R,11aS)-11a-Benzyl-9-ethyl-9-hydroxy-N-(2-methylpyridin-3-yl)-5-oxo-5,7,7a,8,9,10,11,11a-octahydrodibenzo[c,e]oxepine-3-carboxamide;
(7aR,9R,11aS)-11a-Benzyl-9-ethyl-9-hydroxy-6-oxo-6,7,7a,8,9,10,11,11a-octahydro-5H-dibenzo[a,c]cycloheptene-3-carboxylic acid (2-methyl-pyridin-3-yl)-amide;
(7aS,9R,11aR)-11a-Ethyl-9-hydroxy-N-(2-methylpyridin-3-yl)-9-propyl-6,7,7a,8,9,10,11,11a-octahydrodibenzo[b,d]oxepine-3-carboxamide; compound with (7aR,9S,11aS)-11a-ethyl-9-hydroxy-N-(2-methylpyridin-3-yl)-9-propyl-6,7,7a,8,9,10,11,11a-octahydrodibenzo[b,d]oxepine-3-carboxamide;
(7aR,9S,11aS)-11a-ethyl-9-hydroxy-N-(2-methylpyridin-3-yl)-9-propyl-6,7,7a,8,9,10,11,11a-octahydrodibenzo[b,d]oxepine-3-carboxamide;
(7aS,9R,11aR)-11a-ethyl-9-hydroxy-N-(2-methylpyridin-3-yl)-9-propyl-6,7,7a,8,9,10,11,11a-octahydrodibenzo[b,d]oxepine-3-carboxamide;
(7aS,9R,11aR)-11a-Ethyl-9-hydroxy-9-phenyl-6,7,7a,8,9,10,11,11a-octahydro-5H-dibenzo[a,c]cycloheptene-3-carboxylic acid (2-methyl-pyridin-3-yl)-amide;
(7aR,9S,11aS)-11a-ethyl-9-hydroxy-9-phenyl-6,7,7a,8,9,10,11,11a-octahydro-5H-dibenzo[a,c]cycloheptene-3-carboxylic acid (2-methyl-pyridin-3-yl)-amide;
(7aS,9R,10R,11aR)-11a-Ethyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide;
(7aR,9S,10S,11aS)-11a-Ethyl-9,10-dihydroxy-9-phenyl-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2-amino-phenyl)-amide;
(3R,4aS,11bS)-9-(1H-benzoimidazol-2-yl)-11b-benzyl-3-ethyl-2,3,4,4a,5,6,7,11b-octahydro-1H-dibenzo[a,c]cyclohepten-3-ol;
(7aS,9R,11aR)-11a-Ethyl-9-hydroxy-9-propyl-7a,8,9,10,11,11a-hexahydro-7H-dibenzo[a,c]cycloheptene-3-carboxylic acid (2-methyl-pyridin-3-yl)-amide compound with (7aR,9S,11aS)-11a-ethyl-9-hydroxy-9-propyl-7a,8,9,10,11,11a-hexahydro-7H-dibenzo[a,c]cycloheptene-3-carboxylic acid (2-methyl-pyridin-3-yl)-amide;
(7aS,9R,11aR)-11a-Ethyl-9-hydroxy-9-propyl-7a,8,9,10,11,11a-hexahydro-7H-dibenzo[a,c]cycloheptene-3-carboxylic acid (2-methyl-pyridin-3-yl)-amide;
(7aR,9S,11aS)-11a-ethyl-9-hydroxy-9-propyl-7a,8,9,10,11,11a-hexahydro-7H-dibenzo[a,c]cycloheptene-3-carboxylic acid (2-methyl-pyridin-3-yl)-amide;
(7aS,9R,11aS)-11a-Benzyl-9-ethyl-9-hydroxy-7a,8,9,10,11,11a-hexahydro-7H-dibenzo[a,c]cycloheptene-3-carboxylic acid (2-methyl-pyridin-3-yl)-amide;
(7aR,9S,11aR)-11a-Benzyl-9-ethyl-9-hydroxy-7a,8,9,10,11,11a-hexahydro-7H-dibenzo[a,c]cycloheptene-3-carboxylic acid (2-methyl-pyridin-3-yl)-amide;
(7aS,9S,11aR)-11a-Ethyl-9-hydroxy-9-(3,3,3-trifluoro-propyl)-6,7,7a,8,9,10,11,11a-octahydro-5H-dibenzo[a,c]cycloheptene-3-carboxylic acid (2-methyl-pyridin-3-yl)-amide; compound with (7aR,9R,11aS)-11a-ethyl-9-hydroxy-9-(3,3,3-trifluoro-propyl)-6,7,7a,8,9,10,11,11a-octahydro-5H-dibenzo[a,c]cycloheptene-3-carboxylic acid (2-methyl-pyridin-3-yl)-amide;
(7aS,9R,11aR)-11a-Ethyl-9-hydroxy-N-(2-methylpyridin-3-yl)-9-phenyl-6,7,7a,8,9,10,11,11a-octahydrodibenzo[b,d]oxepine-3-carboxamide; compound with (7aR,9S,11aS)-11a-ethyl-9-hydroxy-N-(2-methylpyridin-3-yl)-9-phenyl-6,7,7a,8,9,10,11,11a-octahydrodibenzo[b,d]oxepine-3-carboxamide;
(7aS,9R,10R,11aR)-11a-Ethyl-9,10-dihydroxy-N-(2-methylpyridin-3-yl)-9-phenyl-6,7,7a,8,9,10,11,11a-octahydrodibenzo[b,d]oxepine-3-carboxamide; compound with (7aR,9S,10S,11aS)-11a-ethyl-9,10-dihydroxy-N-(2-methylpyridin-3-yl)-9-phenyl-6,7,7a,8,9,10,11,11a-octahydrodibenzo[b,d]oxepine-3-carboxamide;
(7aS,9R,11aR)-11a-Ethyl-9-propyl-9-hydroxy-N-(2-methylpyridin-3-yl)-5,7,7a,8,9,10,11,11a-octahydrodibenzo[c,e]oxepine-3-carboxamide; compound with (7aR,9S,11aS)-11a-ethyl-9-propyl-9-hydroxy-N-(2-methylpyridin-3-yl)-5,7,7a,8,9,10,11,11a-octahydrodibenzo[c,e]oxepine-3-carboxamide;
(7aR,9S,11aS)-11a-ethyl-9-propyl-9-hydroxy-N-(2-methylpyridin-3-yl)-5,7,7a,8,9,10,11,11a-octahydrodibenzo[c,e]oxepine-3-carboxamide;
(7aS,9R,11aR)-11a-Ethyl-9-propyl-9-hydroxy-N-(2-methylpyridin-3-yl)-5,7,7a,8,9,10,11,11a-octahydrodibenzo[c,e]oxepine-3-carboxamide;
(7aS,9S,11aR)-11a-Ethyl-9-hydroxy-9-isobutyl-5,7,7a,8,9,10,11,11a-octahydro-dibenzo[c,e]oxepine-3-carboxylic acid (2-methyl-pyridin-3-yl)-amide;
(7aS,9R,11aR)-9,11a-diethyl-9-hydroxy-N-(2-methylpyridin-3-yl)-5,7,7a,8,9,10,11,11a-octahydrodibenzo[c,e]oxepine-3-carboxamide;
(7aR,9S,11aS)-9,11a-diethyl-9-hydroxy-N-(2-methylpyridin-3-yl)-5,7,7a,8,9,10,11,11a-octahydrodibenzo[c,e]oxepine-3-carboxamide;
(7aR,9S,11aS)-9,11a-diethyl-9-hydroxy-N-(2-methylpyridin-3-yl)-5,7,7a,8,9,10,11,11a-octahydrodibenzo[c,e]oxepine-3-carboxamide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (4-amino-phenyl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (3-amino-phenyl)-amide;
(7aS,9R,11aS)-11a-Benzyl-9-ethyl-9-hydroxy-6,7,7a,8,9,10,11,11a-octahydro-5H-benzo[c]pyrrolo[1,2-a]azepine-2-carboxylic acid (2-methyl-pyridin-3-yl)-amide; compound with (7aR,9S,11aR)-11a-benzyl-9-ethyl-9-hydroxy-6,7,7a,8,9,10,11,11a-octahydro-5H-benzo[c]pyrrolo[1,2-a]azepine-2-carboxylic acid (2-methyl-pyridin-3-yl)-amide;
(7aS,9R,11aS)-11a-Benzyl-9-ethyl-9-hydroxy-6,7,7a,8,9,10,11,11a-octahydro-5H-dibenzo[a,c]cycloheptene-3-carboxylic acid (2-amino-phenyl)-amide; compound with (7aR,9S,11aR)-11a-benzyl-9-ethyl-9-hydroxy-6,7,7a,8,9,10,11,11a-octahydro-5H-dibenzo[a,c]cycloheptene-3-carboxylic acid (2-amino-phenyl)-amide;
(3R,4aS,11bS)-9-(1H-Benzoimidazol-2-yl)-11b-benzyl-3-ethyl-2,3,4,4a,5,6,7,11b-octahydro-1H-dibenzo[a,c]cyclohepten-3-ol; compound with (3S,4aR,11bR)-9-(1H-benzoimidazol-2-yl)-11b-benzyl-3-ethyl-2,3,4,4 a,5,6,7,11b-octahydro-1H-dibenzo[a,c]cyclohepten-3-ol;
(3R,4aS,11bS)-9-(1H-Benzoimidazol-2-yl)-11b-benzyl-3-ethyl-2,3,4,4a,5,6,7,11b-octahydro-1H-dibenzo[a,c]cyclohepten-3-ol;
(3S,4aR,11bR)-9-(1H-benzoimidazol-2-yl)-11b-benzyl-3-ethyl-2,3,4,4a,5,6,7,11b-octahydro-1H-dibenzo[a,c]cyclohepten-3-ol;
(7aS,9R,11aR)-11a-Ethyl-9-hydroxy-9-propyl-6,7,7a,8,9,10,11,11a-octahydro-5H-dibenzo[a,c]cycloheptene-3-carboxylic acid (2-amino-pyridin-3-yl)-amide; compound with (7aR,9S,11aS)-11a-Ethyl-9-hydroxy-9-propyl-6,7,7a,8,9,10,11,11a-octahydro-5H-dibenzo[a,c]cycloheptene-3-carboxylic acid (2-amino-pyridin-3-yl)-amide;
(7aS,9R,11aS)-11a-Cyclopropylmethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide; compound with (7aR,9S,11aR)-11a-cyclopropylmethyl-9-hydroxy-9-propyl-6,7,7a,8,9,10,11,11a-octahydro-5H-dibenzo[a,c]cycloheptene-3-carboxylic acid (2-methyl-pyridin-3-yl)-amide; or
(7aS,9R,11aR)-11a-Ethyl-9-hydroxy-9-propyl-6,7,7a,8,9,10,11,11a-octahydro-5H-dibenzo[a,c]cycloheptene-3-carboxylic acid (2-chloro-phenyl)-amide.

14. The compound of claim 1 wherein the compound is of Formula (I)c or Formula (I)d

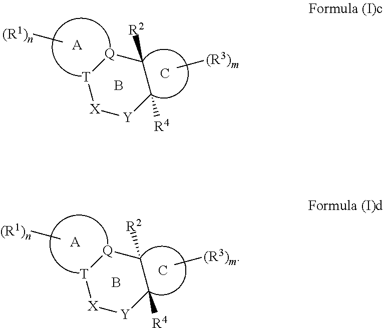

15. The compound of claim 14 wherein Ring A is optionally substituted phenyl, optionally substituted pyrazolyl or optionally substituted pyrrolyl.

16. The compound of claim 15 wherein Ring C is optionally substituted cyclohexyl or optionally substituted cyclohexenyl.

17. The compound of claim 16 wherein X is —C(R⁵)₂—, —C(R⁵)—, —C(═O)—, —O— or —N(R^{a})—.

18. The compound of claim 17 wherein Y is —C(R⁵)₂C(R⁵)₂—, —C(R⁵)C(R⁵)₂—, —C(R⁵)₂C(R⁵)—, —OC(R⁵)₂—, —N(R^{a})C(R⁵)₂—, —C(R⁵)₂N(R^{a})—, —C(═O)C(R⁵)₂—, —C(R⁵)₂C(═O)—, —O—C(═O)—, —C(═O)—O—, —C(R⁵)₂—O—, —O—C(R⁵)₂— or —O—C(R⁵)(R^{b}).

19. The compound of claim 18 wherein R¹ is —COOR^{a}, OR^{a}, —O-optionally substituted (C₁-C₃)alkylene-optionally substituted phenyl, —O-optionally substituted (C₁-C₃)alkylene-optionally substituted pyridinyl, optionally substituted (C₁-C₃)alkyl, —C(O)N(R^{a})(CH₂)ᵣ—R^{b}, or —N(R^{a})C(O)(CH₂)ᵣ—R^{b}.

20. The compound of claim 19 wherein R² is —(CH₂)ᵣ-optionally substituted phenyl, -optionally substituted (C₃-C₆)cycloalkyl, optionally substituted (C₁-C₃)alkyl, or —(CH₂)ᵣ-optionally substituted heteroaryl.

21. The compound of claim 20 wherein R³ is independently H, —CF₃, optionally substituted (C₂-C₆)alkynyl, oxo, —OR^{a}, —OP(═O)(OH)(OH), optionally substituted (C₁-C₄)alkyl, —CH₂-optionally substituted cyclopropyl, or optionally substituted phenyl.

22. The compound of claim 21 wherein R^{b} is optionally substituted phenyl, -optionally substituted pyrimidinyl, optionally substituted pyridinyl, optionally substituted pyrazolyl or optionally substituted tetrazolyl.

23. The compound of claim 22 wherein Q is C.

24. The compound of claim 23 wherein T is C.

25. A compound of Formula (I) wherein the compound is
(4aS,11bS)-11b-Benzyl-9-hydroxy-1,2,4,4 a,5,6,7,11b-octahydro-dibenzo[a,c]cyclohepten-3-one; compound with (4aR,11bR)-11b-benzyl-9-hydroxy-1,2,4,4a,5,6,7,11b-octahydro-dibenzo[a,c]cyclohepten-3-one;
(7aR,11aS)-11a-Benzyl-9-oxo-6,7,7a,8,9,10,11,11a-octahydro-5H-dibenzo[a,c]cycloheptene-3-carboxylic acid (2-methyl-pyridin-3-yl)-amide; compound with (7aS,11aR)-11a-benzyl-9-oxo-6,7,7a,8,9,10,11,11a-octahydro-5H-dibenzo[a,c]cycloheptene-3-carboxylic acid (2-methyl-pyridin-3-yl)-amide;
(7aR,9R,11aS)-11a-Benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide; compound with (7aS,9S,11aR)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide;
(7aR,9S,11aS)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide; compound with (7aS,9R,11aR)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide;
(7aS,9R,11aS)-11a-Benzyl-9-hydroxy-N-(2-methylpyridin-3-yl)-6-oxo-9-(trifluoromethyl)-6,7,7a,8,9,10,11,11a-octahydro-5H-dibenzo[b,c1]azepine-3-carboxamide;
(7aR,9R,11aS)-11a-Benzyl-9-hydroxy-9-trifluoromethyl-6,7,7a,8,9,10,11,11a-octahydro-5H-dibenzo[a,c]cycloheptene-3-carboxylic acid (2-methyl-2H-pyrazol-3-yl)-amide;
(7aR,9R,11aS)-11a-Benzyl-9-hydroxy-9-trifluoromethyl-6,7,7a,8,9,10,11,11a-octahydro-5H-dibenzo[a,c]cycloheptene-3-carboxylic acid (3-methyl-pyridin-4-yl)-amide;
(7aS,9S,11aR)-11a-Benzyl-9-hydroxy-9-trifluoromethyl-6,7,7a,8,9,10,11,11a-octahydro-5H-dibenzo[a,c]cycloheptene-3-carboxylic acid (2-methyl-2H-pyrazol-3-yl)-amide;
(7aS,9R,11aS)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide;
(7aS,9R,11aR)-11a-benzyl-9-hydroxy-9-methyl-6,7,7a,8,9,10,11,11a-octahydro-5H-dibenzo[a,c]cycloheptene-3-carboxylic acid (2-methyl-pyridin-3-yl)-amide;
(7aR,9R,11aS)-11a-Benzyl-9-hydroxy-9-trifluoromethyl-6,7,7a,8,9,10,11,11a-octahydro-5H-dibenzo[a,c]cycloheptene-3-carboxylic acid (2-methyl-pyridin-3-yl)-amide;
(7aS,9S,11aR)-11a-Benzyl-9-hydroxy-9-trifluoromethyl-6,7,7a,8,9,10,11,11a-octahydro-5H-dibenzo[a,c]cycloheptene-3-carboxylic acid (2-methyl-pyridin-3-yl)-amide;
(3R,4aR,11bS)-11b-Benzyl-3-ethyl-3-hydroxy-6-methyl-N-(2-methylpyridin-3-yl)-7-oxo-2,3,4,4a,5,6,7,11b-octahydro-1H-dibenzo[c,e]azepine-9-carboxamide;
(7aR,9R,11aS)-11a-Benzyl-9-hydroxy-N-(2-methylpyridin-3-yl)-5-oxo-9-(trifluoromethyl)-5,7,7a,8,9,10,11,11a-octahydrodibenzo[c,e]oxepine-3-carboxamide;
(7aR,9R,11aS)-11a-Benzyl-9-hydroxy-N-(2-methylpyridin-3-yl)-9-(trifluoromethyl)-5,7,7a,8,9,10,11,11a-octahydrodibenzo[c,e]oxepine-3-carboxamide;
(3R,4aR,11bS)-11b-Benzyl-3-hydroxy-6-methyl-N-(2-methylpyridin-3-yl)-3-(trifluoromethyl)-2,3,4,4a,5,6,7,11b-octahydro-1H-dibenzo[c,e]azepine-9-carboxamide; or
(7aR,9R,11aS)-11a-Benzyl-9-hydroxy-5-oxo-9-trifluoromethyl-6,7,7a,8,9,10,11,11a-octahydro-5H-dibenzo[a,c]cycloheptene-3-carboxylic acid (2-methyl-pyridin-3-yl)-amide.

26. The compound
11b-Benzyl-9-methoxy-1,2,5,6,7,11b-hexahydro-dibenzo[a,c]cyclohepten-3-one;
11b-Benzyl-9-hydroxy-1,2,5,6,7,11b-hexahydro-dibenzo[a,c]cyclohepten-3-one;
(9R,11aS)-11a-Benzyl-9-hydroxy-9-methyl-6,7,9,10,11,11a-hexahydro-5H-dibenzo[a,c]cycloheptene-3-carboxylic acid (2-methyl-pyridin-3-yl)-amide; compound with (9S,11aR)-11a-benzyl-9-hydroxy-9-methyl-6,7,9,10,11,11a-hexahydro-5H-dibenzo[a,c]cycloheptene-3-carboxylic acid (2-methyl-pyridin-3-yl)-amide; or
(4aS,9aS)-4a-Benzyl-octahydro-benzocycloheptene-2,5-dione; compound with (4aR,9aR)-4a-benzyl-octahydro-benzocycloheptene-2,5-dione.

27. A pharmaceutical composition comprising a compound of Formula (I) and a pharmaceutically acceptable carrier or excipient.

28. A method of treating a disease or condition comprising administering a therapeutically effective amount of a compound of Formula (I).

29. The method of claim 28 wherein the disease or condition to be treated is acquired immunodeficiency syndrome (AIDS), acute adrenal insufficiency, addiction, Addison's Disease, adrenal function, allergic rhinitis, allergies, Alzheimer's, anorexia, angioneurotic edema, ankylosing spondylitis, anxiety, asthma, auto-immunity, autoimmune chronic active hepatitis, autoimmune diseases, blepharitis, bursitis, cachexia, cardiovascular disease, cerebral edema, choroidal neovascularization due to age-related macular degeneration, chronic kidney disease, chronic obstructive pulmonary disease, chronic primary adrenal insufficiency, chronic retinal detachment, compulsive behavior, congenital adrenal hyperplasia, cognitive dysfunction, conjunctivitis, cirrhosis, Crohn's disease, Cushing's syndrome, depression, diabetes, diabetes mellitus, diabetic microangiopathy, diabetic neuropathy, diabetic retinopathy, dry eye syndrome, frailty, giant cell arteritis, glaucoma, granulomatous polyarteritis, hay fever, hepatitis, HPA axis suppression and regulation, human immunodeficiency virus (HIV), hypercalcemia, hypercortisolemia, hypergylcemia, hypertension, immune proliferation/apoptosis, immunodeficiency, immunomodulation, inflammation, inflammation of the eye, inflammatory bowel disease, inhibition of myeloid cell lines, insulin dependent diabetes mellitus, insulin-dependent diabetes mellitus glaucoma, insulin resistance, iridocyclitis, juvenile idiopathic arthritis, juvenile rheumatoid arthritis, leukemia, Little's syndrome, lupus, lymphoma, macular degeneration, macular edema, a malignancy, medical catabolism, multi-drug resistance, multiple sclerosis, neurodgeneration, obesity, ocular or macular edema, ocular neovascular disease, organ transplantation, modulation of the Th1/Th2 cytokine balance, optic neuritis, optic pits, neuropathy, osteoarthritis, osteoporosis, Parkinson's, plaque psoriasis, polyarteritis nodosa, post-laser treatment complications, post-surgical bone fracture, post-traumatic stress syndrome, prevention of muscle frailty, psoriasis, psoriatic arthritis, psychosis, regulation of carbohydrate, protein and lipid metabolism, regulation of electrolyte and water balance, regulation of functions of the cardiovascular, kidney, central nervous, immune, or skeletal muscle systems, retinopathy of prematurity, rheumatic fever, rheumatoid arthritis, rhinitis, scleritis, secondary adrenal insufficiency, stroke and spinal cord injury, sympathetic ophthalmia, systemic lupus erythematosus, Syndrome X, tendonitis, thrombocytopenia, tissue rejection, ulcerative colitis, urticaria, uveitis, viral infection, Wegener's granulomatosis or wound healing.

30. A process for the preparation of a compound of Formula 2 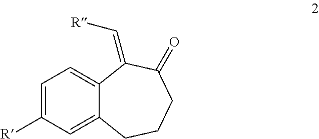
comprising the step of reacting compound of Formula 1 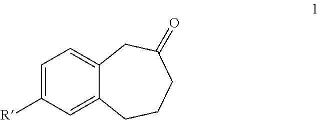
with a base until the reaction is substantially complete, then reacting the anion with acetaldehyde to form a compound of Formula 2 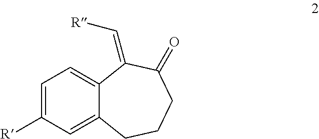
wherein
R′ is alkoxy and
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl;
wherein r is independently 0, 1 or 2.

31. The process according to claim 30, further comprising the step of warming.

32. A process for the preparation of a compound of Formula 3 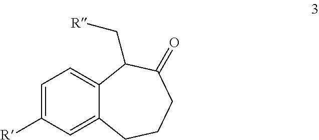
comprising the step of reacting compound of Formula 2 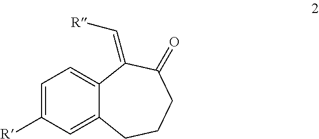
with a catalyst and hydrogen until the reaction is substantially complete to form a compound of Formula 3 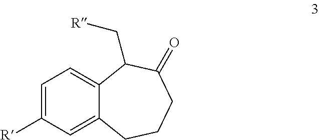
wherein
R′ is alkoxy and
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl;
wherein r is independently 0, 1 or 2.

33. A process for the preparation of a compound of Formula 4 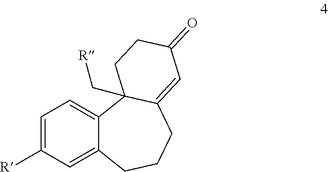
comprising the step of reacting compound of Formula 3 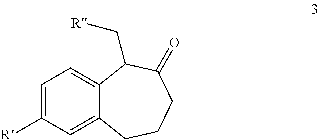
with a ketone and a base until the reaction is substantially complete to form a compound of Formula 4 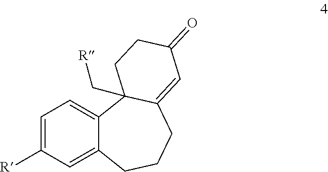
wherein
R′ is alkoxy and
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl.

34. A process for the preparation of a compound of Formula 6 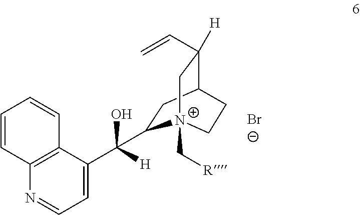
comprising the step of reacting compound of Formula 5 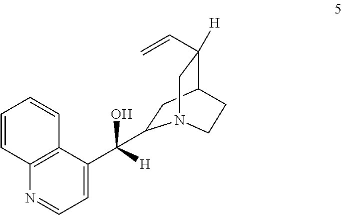
with 1-(bromomethyl)-2-fluoro-4-(trifluoromethyl)benzene until the reaction is substantially complete to form a compound of Formula 6 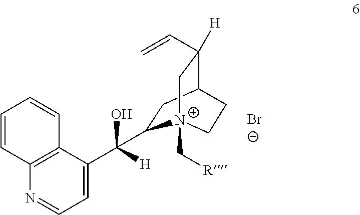
wherein R″″ is arylhalide.

35. A process for the preparation of compounds of Formulas 3a and 3b 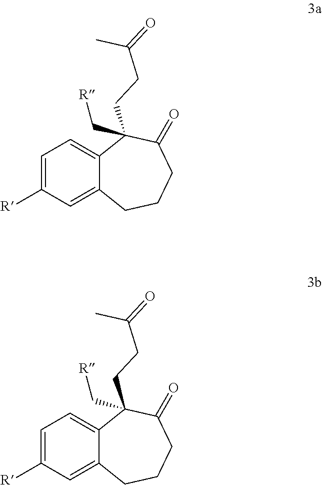
comprising reacting a compound of Formula 3 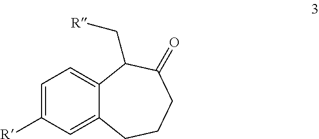
with an eneone, a base and a compound of Formula 6 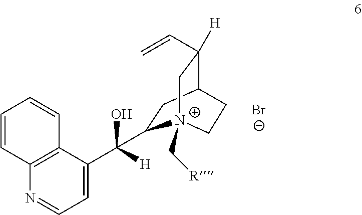
until the reaction is substantially complete to form compounds of Formulas 3a and 3b 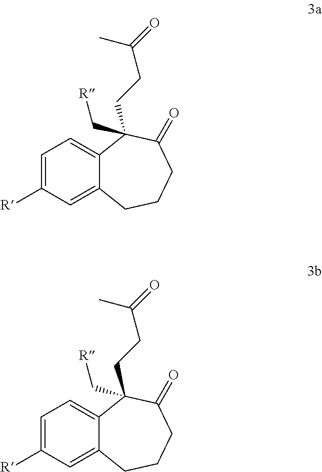
wherein
R′ is alkoxy;
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl; and
R″″ is arylhalide.

36. A process for preparing compounds of Formula 3c and 3d 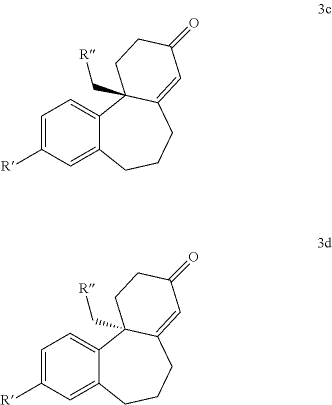
comprising reacting compounds of Formulas 3a and 3b 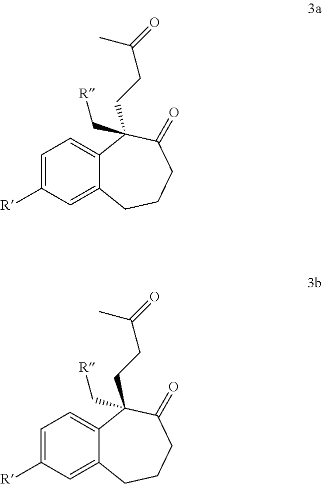
with a base until the reaction is substantially complete to form a compound of Formulas 3c and 3d 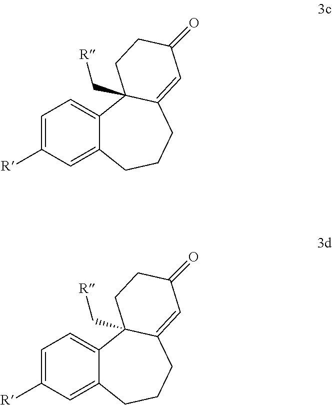
wherein
R′ is alkoxy and
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl.

37. A process for preparing a compound of Formula 4a 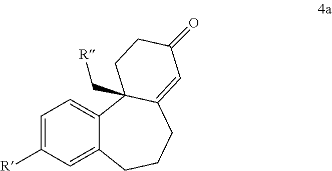
comprising fractional crystallization of formulas 3c and 3d 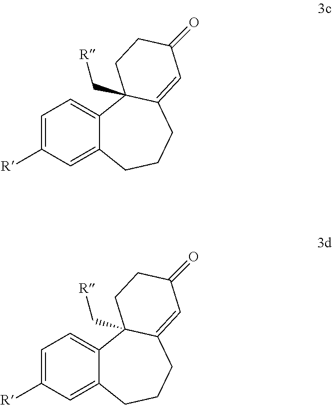
until the reaction is substantially complete to form a compound of Formula 4a 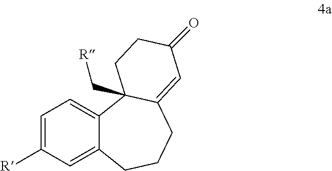
wherein
R′ is alkoxy and
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl.

38. A process for preparing a compound of Formula 7 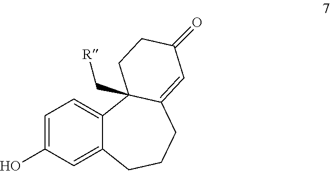
comprising reacting a compound of Formula 4a 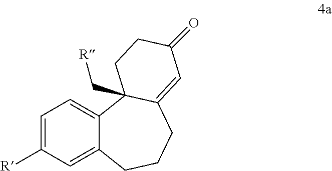
with an acid and methionine until the reaction is substantially complete to form a compound of Formula 7 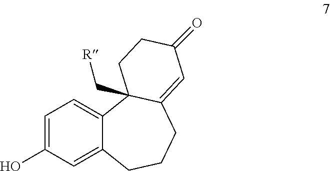
wherein
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl.

39. The process according to claim 38, wherein the acid is methanesulfonic acid.

40. A process for preparing a compound of Formula 8 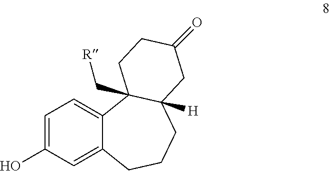
comprising reacting a compound of Formula 7 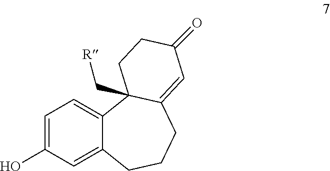
with hydrogen and a catalyst until the reaction is substantially complete to form a compound of Formula 8 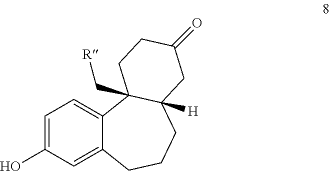
wherein
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl.

41. A process for preparing a compound of Formula 9 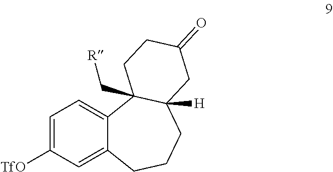
comprising reacting a comnound of Formula 8 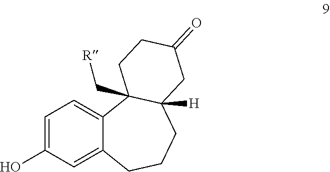
with a triflating reagent N-phenylbis(trifluoromethanesulfonimide and a base until the reaction is substantially complete to form a compound of Formula 9 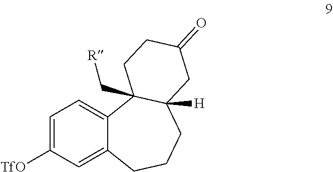
wherein
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl.

42. The process according to claim 41 wherein the triflating reagent is N-phenylbis(trifluoromethanesulfonimide.

43. A process for preparing a compound of Formula 10 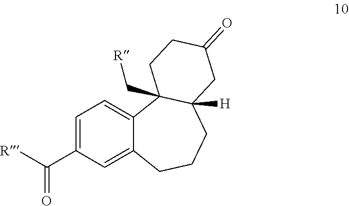
comprising reacting a compound of Formula 9 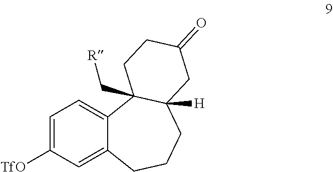
with carbon monoxide and a catalyst until the reaction is substantially complete to form a compound of Formula 10 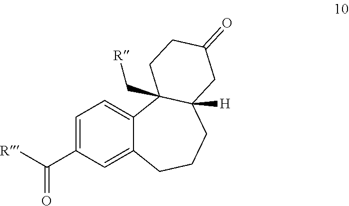
wherein
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl and
R′″ is optionally substituted aminoaryl, optionally substituted aminoheterocyclyl, optionally substituted aminoheteroaryl or optionally substituted aminocycloalkyl.

44. A process for preparing a compound of Formula 11 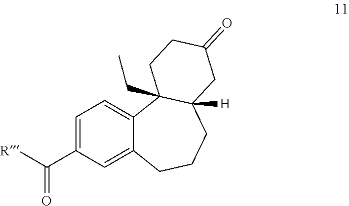
comprising reacting a compound of Formula 10 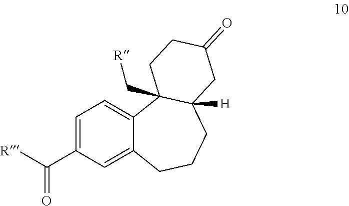
with a base until the reaction is substantially complete, then coupling to an amine to form a compound of Formula 11 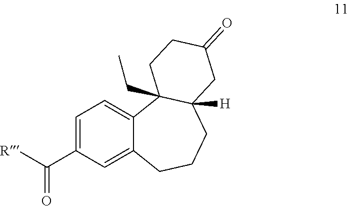
wherein
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl and
R′″ is optionally substituted aminoaryl, optionally substituted aminoheterocyclyl, optionally substituted aminoheteroaryl or optionally substituted aminocycloalkyl.

45. A process for preparing a compound of Formula 12 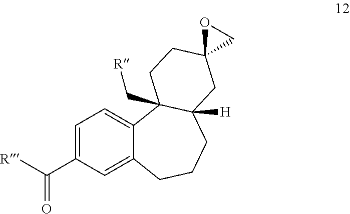
comprising reacting a compound of Formula 11 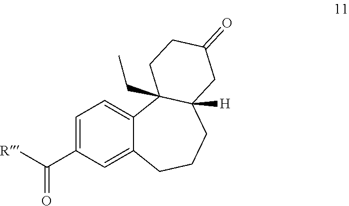
with a base and trimethylsulfoxonium halide until the reaction is substantially complete to form a compound of Formula 12 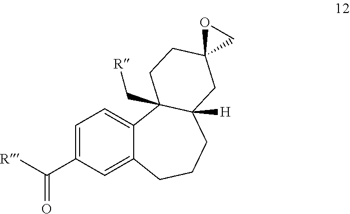
wherein
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl and
R′″ is optionally substituted aminoaryl, optionally substituted aminoheterocyclyl, optionally substituted aminoheteroaryl or optionally substituted aminocycloalkyl.

46. A process for preparing a compound of Formula 13 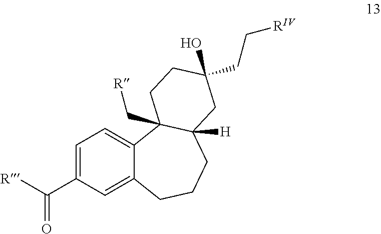
comprising reacting a compound of Formula 12 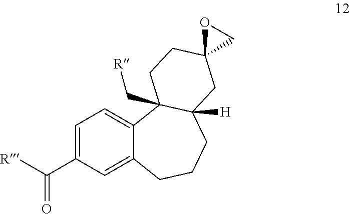
with a metal halide until the reaction is substantially complete to form a compound of Formula 13 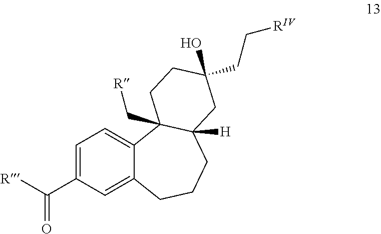
wherein
R″ is CF₃, —(CH₂)ᵣ-optionally substituted aryl, —(CH₂)ᵣ-optionally substituted (C₃-C₆)cycloalkyl, optionally substituted (C₁-C₃)alkyl, or —(CH₂)ᵣ-optionally substituted heteroaryl;
R′″ is optionally substituted aminoaryl, optionally substituted aminoheterocyclyl, optionally substituted aminoheteroaryl or optionally substituted aminocycloalkyl and
R^{IV} is H, optionally substituted (C₁-C₃)alkyl, OH or —O-optionally substituted (C₁-C₃)alkyl.
